(19) United States
(12) Patent Application Publication
(10) Pub. No.: US 2024/0208991 A1
(43) Pub. Date: Jun. 27, 2024

(54) NOVEL COMPOUND AND ORGANIC LIGHT EMITTING DEVICE COMPRISING THE SAME

(71) Applicant: LG CHEM, LTD., Seoul (KR)

(72) Inventors: Minjun KIM, Daejeon (KR); Young Seok KIM, Daejeon (KR); Dong Hoon LEE, Daejeon (KR); Sang Duk SUH, Daejeon (KR); Woo Han KIM, Daejeon (KR)

(21) Appl. No.: 18/278,828

(22) PCT Filed: Aug. 4, 2022
(86) PCT No.: PCT/IB2022/057237

§ 371 Date: Aug. 24, 2023

(30) Foreign Application Priority Data
Aug. 4, 2021 (KR) 10-2021-0102550

(51) Int. Cl. C07D 498/04; C07D 513/04; C07D 519/00; H10K 50/11; H10K 85/60

(52) U.S. Cl. CPC C07D 498/04; C07D 513/04; C07D 519/00; H10K 50/11; H10K 85/656; H10K 85/6572

(57) ABSTRACT

Provided is a novel compound of Chemical Formula 1 or Chemical Formula 2:

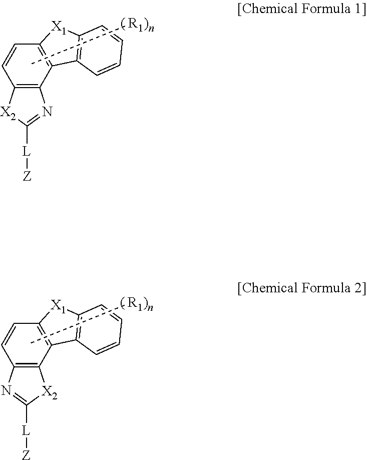

wherein X₁ and X₂ are each independently O or S; each R₁ is independently hydrogen, deuterium, a substituted or unsubstituted C₆₋₆₀ aryl or C₂₋₆₀ heteroaryl containing any one or more of N, O, and S; L is a direct bond or a substituted or unsubstituted C₆₋₆₀ arylene or C₂₋₆₀ heteroarylene containing any one or more of N, O, and S; and Z is the following Chemical Formula 3 or Chemical Formula 4:

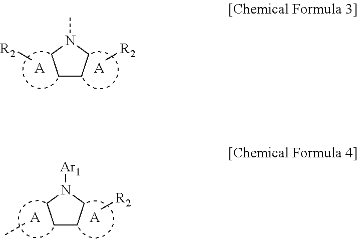

wherein each A is independently a substituted or unsubstituted C₆₋₆₀ aromatic ring or C₂₋₆₀ heteroaromatic ring containing any one or more of N, O, and S fused with an adjacent five-membered ring; and the other substituents are as defined in the specification; and an organic light emitting device including the same.

### CROSS-REFERENCE TO RELATED APPLICATION(S)

This application is a National Stage Application of International Application No. PCT/IB2022/057237 filed on Aug. 4, 2022, which claims the benefit of Korean Patent Application No. 10-2021-0102550 filed on Aug. 4, 2021 with the Korean Intellectual Property Office, the disclosure of which is incorporated herein by reference in its entirety.

### TECHNICAL FIELD

The present disclosure relates to a novel compound and an organic light emitting device comprising the same.

### BACKGROUND

In general, an organic light emitting phenomenon refers to a phenomenon where electric energy is converted into light energy by using an organic material. The organic light emitting device using the organic light emitting phenomenon has characteristics such as a wide viewing angle, an excellent contrast, a fast response time, an excellent luminance, driving voltage and response speed, and thus many studies have proceeded.

The organic light emitting device generally has a structure which comprises an anode, a cathode, and an organic material layer interposed between the anode and the cathode. The organic material layer frequently has a multilayered structure that comprises different materials in order to enhance efficiency and stability of the organic light emitting device, and for example, the organic material layer can be formed of a hole injection layer, a hole transport layer, a light emitting layer, an electron transport layer, an electron injection layer, and the like. In the structure of the organic light emitting device, if a voltage is applied between two electrodes, the holes are injected from an anode into the organic material layer and the electrons are injected from the cathode into the organic material layer, and when the injected holes and electrons meet each other, an exciton is formed, and light is emitted when the exciton falls to a ground state again.

There is a continuous need to develop a new material for the organic materials used in the organic light emitting devices as described above.

### PRIOR ART LITERATURE

### Patent Literature

(Patent Literature 1) Korean Unexamined Patent Publication No. 10-2000-0051826

### DETAILED DESCRIPTION OF THE INVENTION

### Technical Problem

It is an object of the present disclosure to provide a novel compound and an organic light emitting device comprising the same.

### Technical Solution

According to an aspect of the present disclosure, provided is a compound of the following Chemical Formula 1 or Chemical Formula 2:

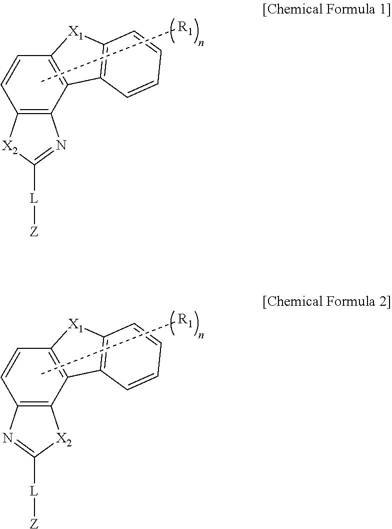

wherein, in Chemical Formulae 1 and 2:
X₁ and X₂ are each independently O or S;
each R₁ is independently hydrogen, deuterium, a substituted or unsubstituted C₆₋₆₀ aryl, or a substituted or unsubstituted C₂₋₆₀ heteroaryl containing any one or more heteroatoms selected from the group consisting of N, O, and S;
L is a direct bond, a substituted or unsubstituted C₆₋₆₀ arylene, or a substituted or unsubstituted C₂₋₆₀ heteroarylene containing any one or more heteroatoms selected from the group consisting of N, O, and S; and
Z is the following Chemical Formula 3 or Chemical Formula 4:

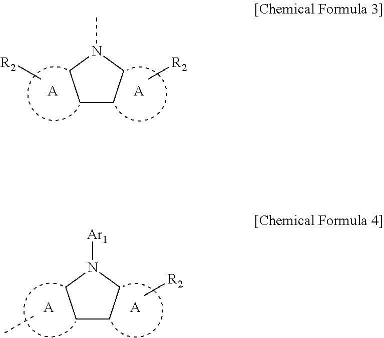

wherein, in Chemical Formulae 3 and 4:
each A is independently a substituted or unsubstituted C₆₋₆₀ aromatic ring fused with an adjacent five-membered ring, or a substituted or unsubstituted C₂₋₆₀ heteroaromatic ring containing any one or more heteroatoms selected from the group consisting of N, O, and S fused with an adjacent five-membered ring;
each R₂ is independently hydrogen, deuterium, a substituted or unsubstituted C₆₋₆₀ aryl, or a substituted or unsubstituted C₂₋₆₀ heteroaryl containing any one or more heteroatoms selected from the group consisting of N, O, and S;
Ar₁ is a substituted or unsubstituted C₆₋₆₀ aryl, or a substituted or unsubstituted C₂₋₆₀ heteroaryl containing any one or more heteroatoms selected from the group consisting of N, O, and S; and
n is an integer of 0 to 6.

According to another aspect of the present disclosure, provided is an organic light emitting device comprising: a first electrode; a second electrode that is opposite to the first electrode; and one or more organic material layers that are between the first electrode and the second electrode, wherein one or more layers of the organic material layers include the compound of Chemical Formula 1 or Chemical Formula 2.

### Advantageous Effects

The above-mentioned compound of Chemical Formula 1 or Chemical Formula 2 can be used as a material for an organic material layer of an organic light emitting device, and can improve the efficiency, achieve low driving voltage and/or improve lifetime characteristics in the organic light emitting device. In particular, the compound of Chemical Formula 1 or Chemical Formula 2 described above can be used as a material for hole injection, hole transport, hole injection and transport, light emission, electron transport, or electron injection.

### BRIEF DESCRIPTION OF THE DRAWINGS

FIG. 1 shows an example of an organic light emitting device comprising a substrate 1, an anode 2, a light emitting layer 3, and a cathode 4.

FIG. 2 shows an example of an organic light emitting device comprising a substrate 1, an anode 2, a hole injection layer 5, a hole transport layer 6, an electron blocking layer 7, a light emitting layer 3, an electron transport layer 8, an electron injection layer 9, and a cathode 4.

### DETAILED DESCRIPTION OF THE EMBODIMENTS

Hereinafter, embodiments of the present disclosure will be described in more detail to facilitate understanding of the invention.

As used herein, the notation [symbol] and [symbol] mean a bond linked to another substituent group.

As used herein, the term “substituted or unsubstituted” means being unsubstituted or substituted with one or more substituents selected from the group consisting of deuterium, a halogen group, a nitrile group, a nitro group, a hydroxy group, a carbonyl group, an ester group, an imide group, an amino group, a phosphine oxide group, an alkoxy group, an aryloxy group, an alkylthioxy group, an arylthioxy group, an alkylsulfoxy group, an arylsulfoxy group, a silyl group, a boron group, an alkyl group, a cycloalkyl group, an alkenyl group, an aryl group, an aralkyl group, an aralkenyl group, an alkylaryl group, an alkylamine group, an aralkylamine group, a heteroarylamine group, an arylamine group, an arylphosphine group, and a heterocyclic group containing at least one of N, O and S atoms, or being unsubstituted or substituted with a substituent to which two or more substituents of the above-exemplified substituents are linked. For example, “a substituent in which two or more substituents are linked” can be a biphenyl group. Namely, a biphenyl group can be an aryl group, or it can be interpreted as a substituent in which two phenyl groups are linked.

In the present disclosure, the carbon number of a carbonyl group is not particularly limited, but is preferably 1 to 40. Specifically, the carbonyl group can be a substituent group having the following structural formulas, but is not limited thereto:

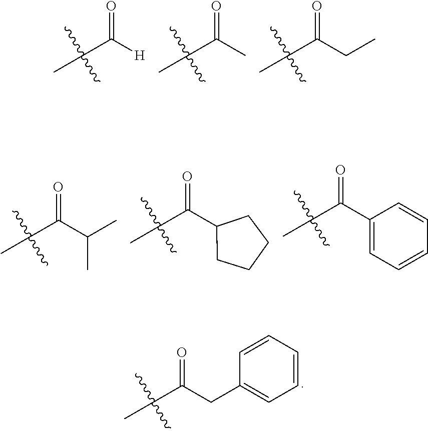

In the present disclosure, an ester group can have a structure in which oxygen of the ester group can be substituted by a straight-chain, branched-chain, or cyclic alkyl group having 1 to 25 carbon atoms, or an aryl group having 6 to 25 carbon atoms. Specifically, the ester group can be a substituent group having the following structural formulas, but is not limited thereto:

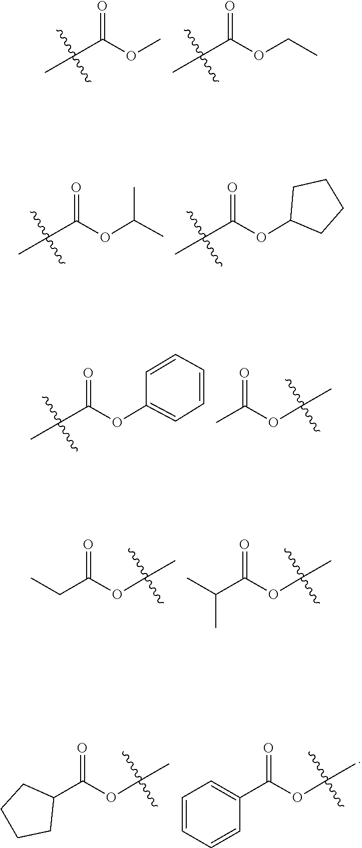

In the present disclosure, the carbon number of an imide group is not particularly limited, but is preferably 1 to 25. Specifically, the imide group can be a substituent group having the following structural formulas, but is not limited thereto:

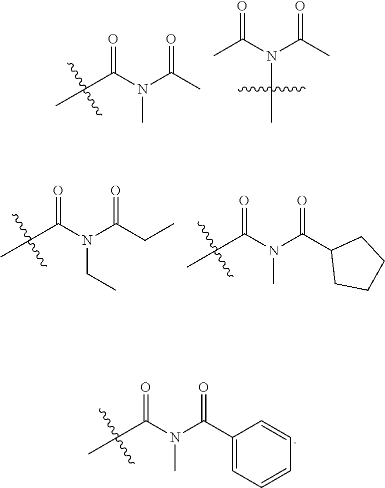

In the present disclosure, a silyl group specifically includes a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, and the like, but is not limited thereto.

In the present disclosure, a boron group specifically includes a trimethylboron group, a triethylboron group, a t-butyldimethylboron group, a triphenylboron group, and a phenylboron group, but is not limited thereto.

In the present disclosure, examples of a halogen group include fluorine, chlorine, bromine, or iodine.

In the present disclosure, the alkyl group can be straight-chain or branched-chain, and the carbon number thereof is not particularly limited, but is preferably 1 to 40. According to one embodiment, the carbon number of the alkyl group is 1 to 20. According to another embodiment, the carbon number of the alkyl group is 1 to 10. According to another embodiment, the carbon number of the alkyl group is 1 to 6. Specific examples of the alkyl group include methyl, ethyl, propyl, n-propyl, isopropyl, butyl, n-butyl, isobutyl, tert-butyl, sec-butyl, 1-methyl-butyl, 1-ethyl-butyl, pentyl, n-pentyl, isopentyl, neopentyl, tert-pentyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, cyclopentylmethyl, cyclohexylmethyl, octyl, n-octyl, tert-octyl, 1-methylheptyl, 2-ethylhexyl, 2-propylpentyl, n-nonyl, 2,2-dimethylheptyl, 1-ethyl-propyl, 1,1-dimethyl-propyl, isohexyl, 2-methylpentyl, 4-methylhexyl, 5-methylhexyl, and the like, but are not limited thereto.

In the present disclosure, the alkenyl group can be straight-chain or branched-chain, and the carbon number thereof is not particularly limited, but is preferably 2 to 40. According to one embodiment, the carbon number of the alkenyl group is 2 to 20. According to another embodiment, the carbon number of the alkenyl group is 2 to 10. According to still another embodiment, the carbon number of the alkenyl group is 2 to 6. Specific examples thereof include vinyl, 1-propenyl, isopropenyl, 1-butenyl, 2-butenyl, 3-butenyl, 1-pentenyl, 2-pentenyl, 3-pentenyl, 3-methyl-1-butenyl, 1,3-butadienyl, allyl, 1-phenylvinyl-1-yl, 2-phenylvinyl-1-yl, 2,2-diphenylvinyl-1-yl, 2-phenyl-2-(naphthyl-1-yl)vinyl-1-yl, 2,2-bis(diphenyl-1-yl)vinyl-1-yl, a stilbenyl group, a styrenyl group, and the like, but are not limited thereto.

In the present disclosure, a cycloalkyl group is not particularly limited, but the carbon number thereof is preferably 3 to 60. According to one embodiment, the carbon number of the cycloalkyl group is 3 to 30. According to another embodiment, the carbon number of the cycloalkyl group is 3 to 20. According to still another embodiment, the carbon number of the cycloalkyl group is 3 to 6. Specific examples thereof include cyclopropyl, cyclobutyl, cyclopentyl, 3-methylcyclopentyl, 2,3-dimethylcyclopentyl, cyclohexyl, 3-methylcyclohexyl, 4-methylcyclohexyl, 2,3-dimethylcyclohexyl, 3,4,5-trimethylcyclohexyl, 4-tert-butylcyclohexyl, cycloheptyl, cyclooctyl, and the like, but are not limited thereto.

In the present disclosure, an aryl group is not particularly limited, but the carbon number thereof is preferably 6 to 60, and it can be a monocyclic aryl group or a polycyclic aryl group. According to one embodiment, the carbon number of the aryl group is 6 to 30. According to one embodiment, the carbon number of the aryl group is 6 to 20. The aryl group can be a phenyl group, a biphenyl group, a terphenyl group or the like as the monocyclic aryl group, but is not limited thereto. The polycyclic aryl group includes a naphthyl group, an anthracenyl group, a phenanthryl group, a pyrenyl group, a perylenyl group, a chrysenyl group, a fluorenyl group, or the like, but is not limited thereto.

In the present disclosure, the fluorenyl group can be substituted, and two substituents can be connected to each other to form a spiro structure. In the case where the fluorenyl group is substituted,

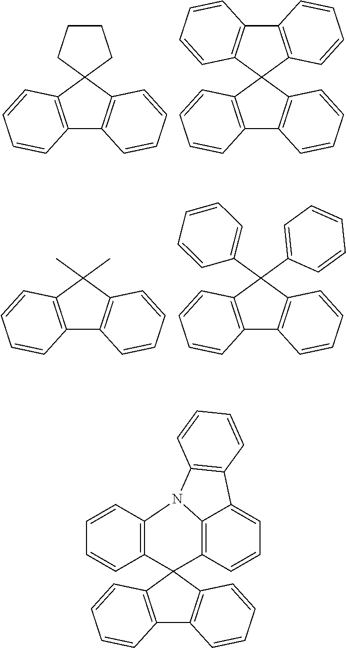

and the like can be formed. However, the structure is not limited thereto.

In the present disclosure, a heterocyclic group is a heterocyclic group containing at least one of O, N, Si and S as a heteroatom, and the carbon number thereof is not particularly limited, but is preferably 2 to 60. Examples of the heterocyclic group include a thiophene group, a furan group, a pyrrole group, an imidazole group, a thiazole group, an oxazol group, an oxadiazol group, a triazol group, a pyridyl group, a bipyridyl group, a pyrimidyl group, a triazine group, an acridyl group, a pyridazine group, a pyrazinyl group, a quinolinyl group, a quinazoline group, a quinoxalinyl group, a phthalazinyl group, a pyridopyrimidinyl group, a pyridopyrazinyl group, a pyrazinopyrazinyl group, an isoquinoline group, an indole group, a carbazole group, a benzoxazole group, a benzoimidazole group, a benzothiazol group, a benzocarbazole group, a benzothiophene group, a dibenzothiophene group, a benzofuranyl group, a phenanthroline group, an isoxazolyl group, a thiadiazolyl group, a phenothiazinyl group, a dibenzofuranyl group, and the like, but are not limited thereto.

In the present disclosure, the aryl group in the aralkyl group, the aralkenyl group, the alkylaryl group and the arylamine group is the same as the above-mentioned examples of the aryl group. In the present disclosure, the alkyl group in the aralkyl group, the alkylaryl group and the alkylamine group is the same as the above-mentioned examples of the alkyl group. In the present disclosure, the heteroaryl in the heteroarylamine can be applied to the above-mentioned description of the heterocyclic group. In the present disclosure, the alkenyl group in the aralkenyl group is the same as the above-mentioned examples of the alkenyl group. In the present disclosure, the above-mentioned description of the aryl group can be applied except that the arylene is a divalent group. In the present disclosure, the above-mentioned description of the heterocyclic group can be applied except that the heteroarylene is a divalent group. In the present disclosure, the above-mentioned description of the aryl group or cycloalkyl group can be applied except that the hydrocarbon ring is not a monovalent group but formed by combining two substituent groups. In the present disclosure, the above-mentioned description of the heterocyclic group can be applied, except that the heterocyclic group is not a monovalent group but formed by combining two substituent groups.

(Compound)

The present disclosure provides the compound of Chemical Formula 1 or Chemical Formula 2.

Preferably, the Chemical Formula 1 is any one selected from the group consisting of Chemical Formulas 1-1 to 1-6, and the Chemical Formula 2 is any one selected from the group consisting of Chemical Formulas 2-1 to 2-6:

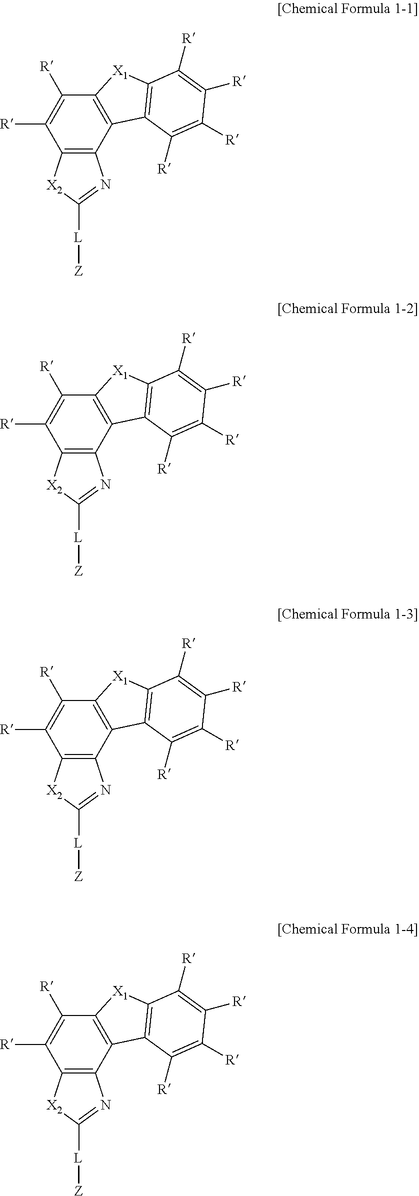

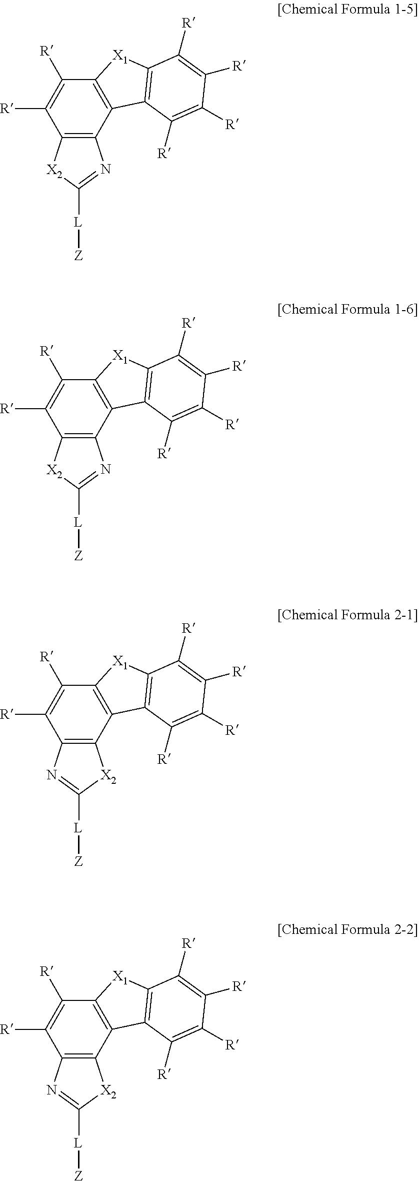

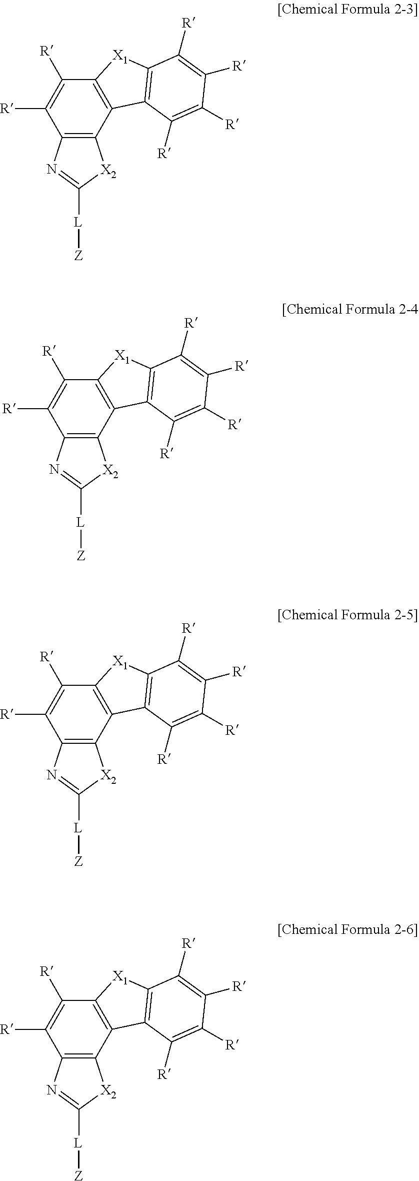

wherein, in Chemical Formulas 1-1 to 1-6 and Chemical Formulas 2-1 to 2-6:
X₁, X₂, R₁, L, and Z are as defined above; and
each R′ is independently hydrogen or deuterium.

Preferably, R₁ is hydrogen, deuterium, phenyl, biphenylyl, naphthyl, phenyl-naphthyl, naphthyl-phenyl, phenanthrenyl, or phenyl-phenanthrenyl.

Preferably, L is a direct bond, or any one selected from the group of the following:

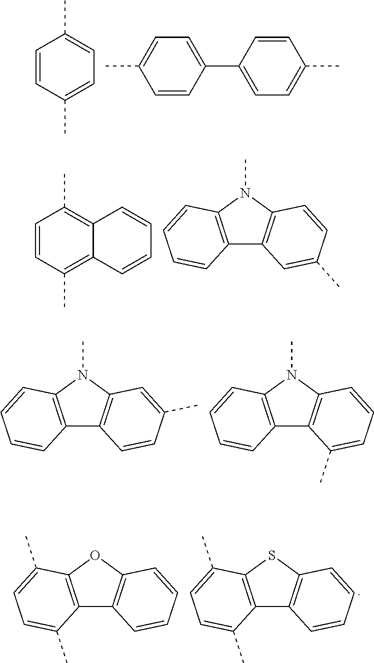

Preferably, each A is independently benzene, naphthalene, carbazole, dibenzofuran, or dibenzothiophene. Here, the carbazole can be 9-phenylcarbazole.

Preferably, each R₂ is independently hydrogen, deuterium, phenyl, naphthyl, 9-phenylcarbazolyl, carbazol-9-yl, or phenanthren-9-yl.

Preferably, Ar₁ is phenyl, biphenylyl, naphthyl, phenyl-naphthyl, or naphthyl-phenyl.

Representative examples of the compound of Chemical Formula 1 or Chemical Formula 2 is as follows:

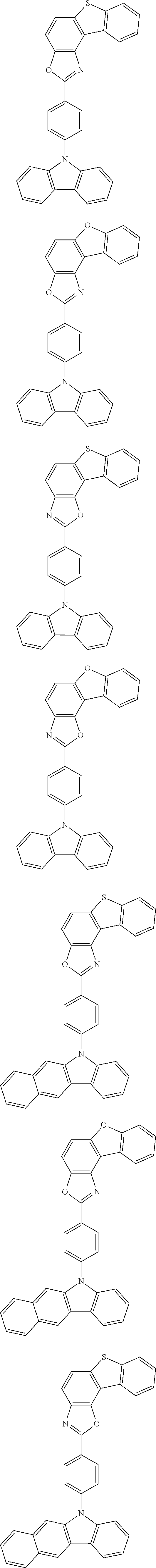

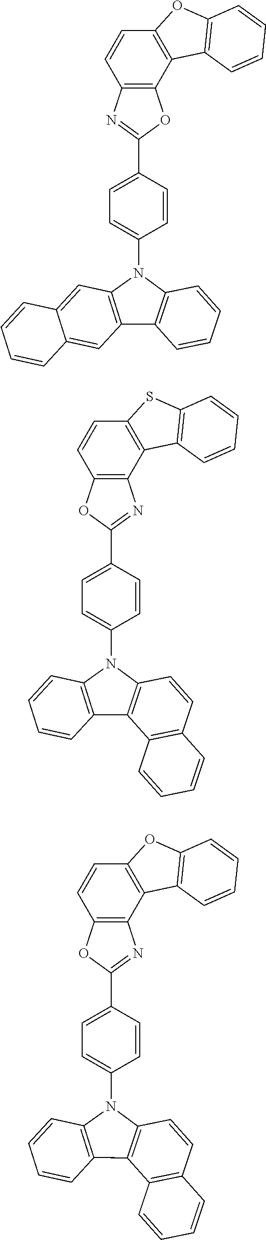

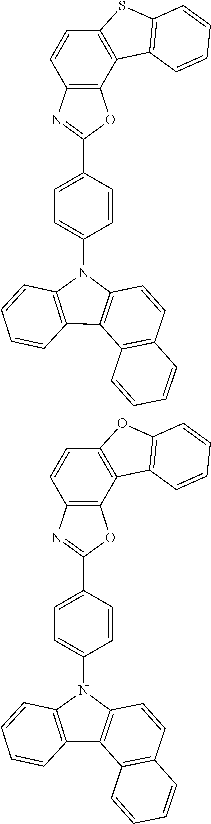

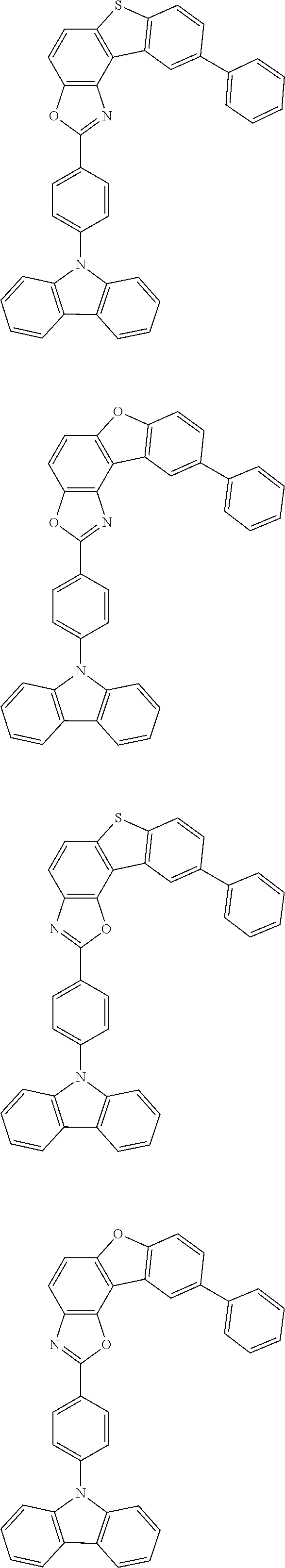 [SMILES: c1ccc(-c2ccc3sc4ccc5oc(-c6ccc(-n7c8ccccc8c8ccccc87)cc6)nc5c4c3c2)cc1 ; c1ccc(-c2ccc3oc4ccc5oc(-c6ccc(-n7c8ccccc8c8ccccc87)cc6)nc5c4c3c2)cc1 ; c1ccc(-c2ccc3sc4ccc5nc(-c6ccc(-n7c8ccccc8c8ccccc87)cc6)oc5c4c3c2)cc1 ; c1ccc(-c2ccc3oc4ccc5nc(-c6ccc(-n7c8ccccc8c8ccccc87)cc6)oc5c4c3c2)cc1]

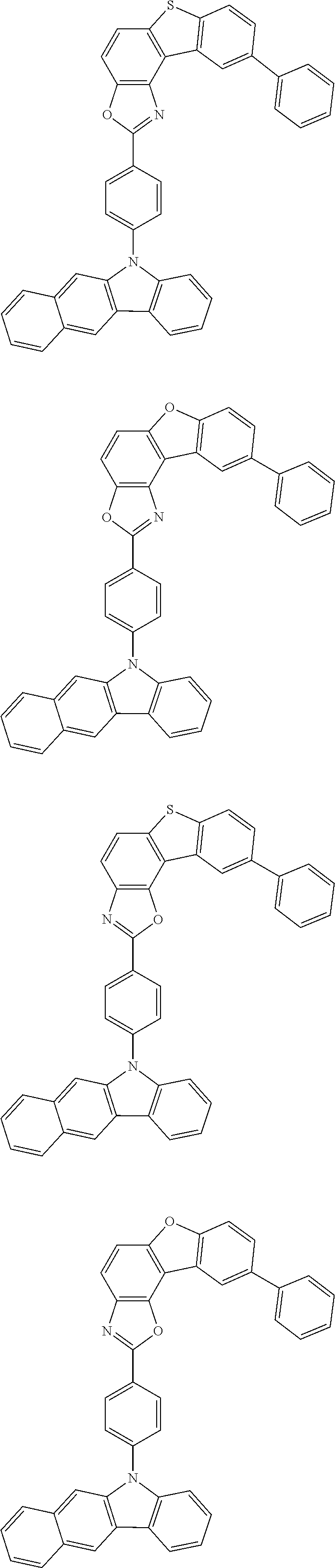 [SMILES: c1ccc(-c2ccc3sc4ccc5oc(-c6ccc(-n7c8ccccc8c8cc9ccccc9cc87)cc6)nc5c4c3c2)cc1 ; c1ccc(-c2ccc3oc4ccc5oc(-c6ccc(-n7c8ccccc8c8cc9ccccc9cc87)cc6)nc5c4c3c2)cc1 ; c1ccc(-c2ccc3sc4ccc5nc(-c6ccc(-n7c8ccccc8c8cc9ccccc9cc87)cc6)oc5c4c3c2)cc1 ; c1ccc(-c2ccc3oc4ccc5nc(-c6ccc(-n7c8ccccc8c8cc9ccccc9cc87)cc6)oc5c4c3c2)cc1]

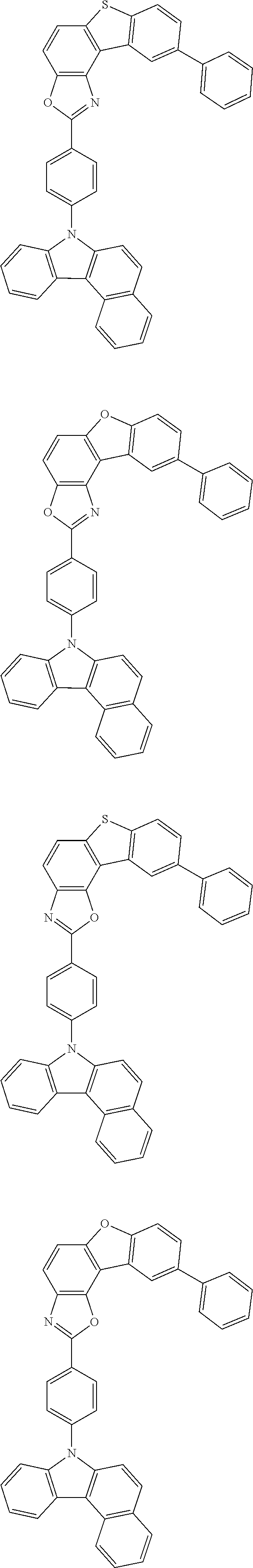 [SMILES: c1ccc(-c2ccc3sc4ccc5oc(-c6ccc(-n7c8ccccc8c8c9ccccc9ccc87)cc6)nc5c4c3c2)cc1 ; c1ccc(-c2ccc3oc4ccc5oc(-c6ccc(-n7c8ccccc8c8c9ccccc9ccc87)cc6)nc5c4c3c2)cc1 ; c1ccc(-c2ccc3sc4ccc5nc(-c6ccc(-n7c8ccccc8c8c9ccccc9ccc87)cc6)oc5c4c3c2)cc1 ; c1ccc(-c2ccc3oc4ccc5nc(-c6ccc(-n7c8ccccc8c8c9ccccc9ccc87)cc6)oc5c4c3c2)cc1]

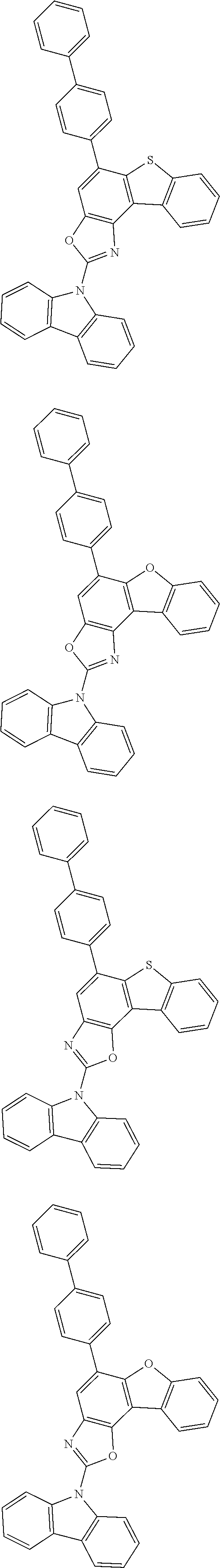 [SMILES: c1ccc(-c2ccc(-c3cc4oc(-n5c6ccccc6c6ccccc65)nc4c4c3sc3ccccc34)cc2)cc1 ; c1ccc(-c2ccc(-c3cc4oc(-n5c6ccccc6c6ccccc65)nc4c4c3oc3ccccc34)cc2)cc1 ; c1ccc(-c2ccc(-c3cc4nc(-n5c6ccccc6c6ccccc65)oc4c4c3sc3ccccc34)cc2)cc1 ; c1ccc(-c2ccc(-c3cc4nc(-n5c6ccccc6c6ccccc65)oc4c4c3oc3ccccc34)cc2)cc1]

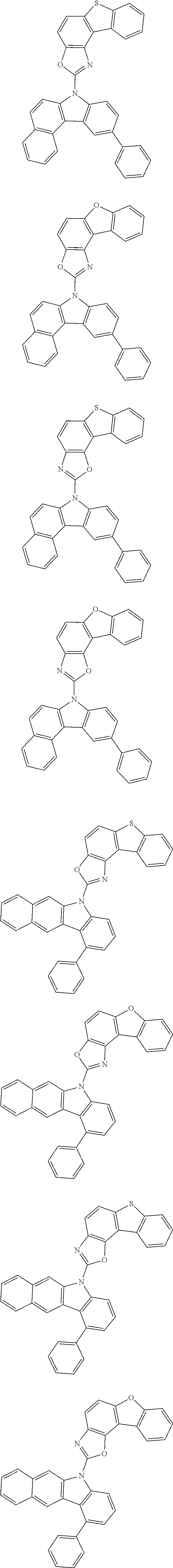

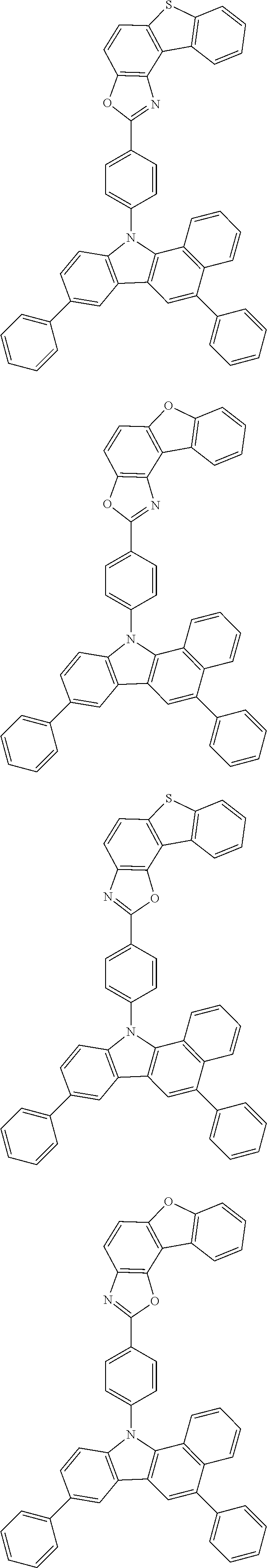

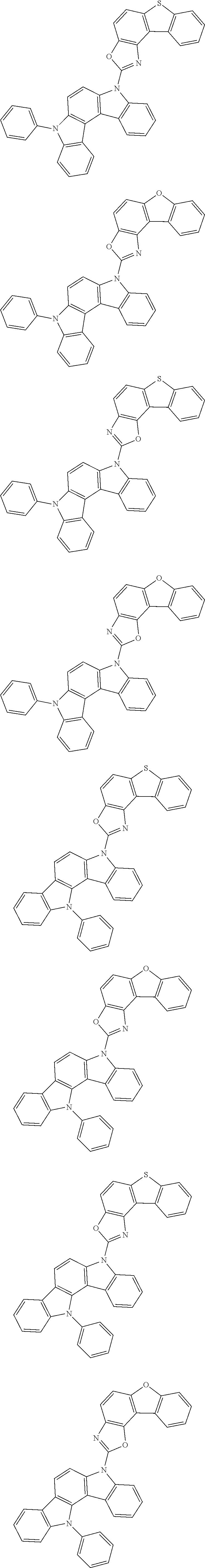

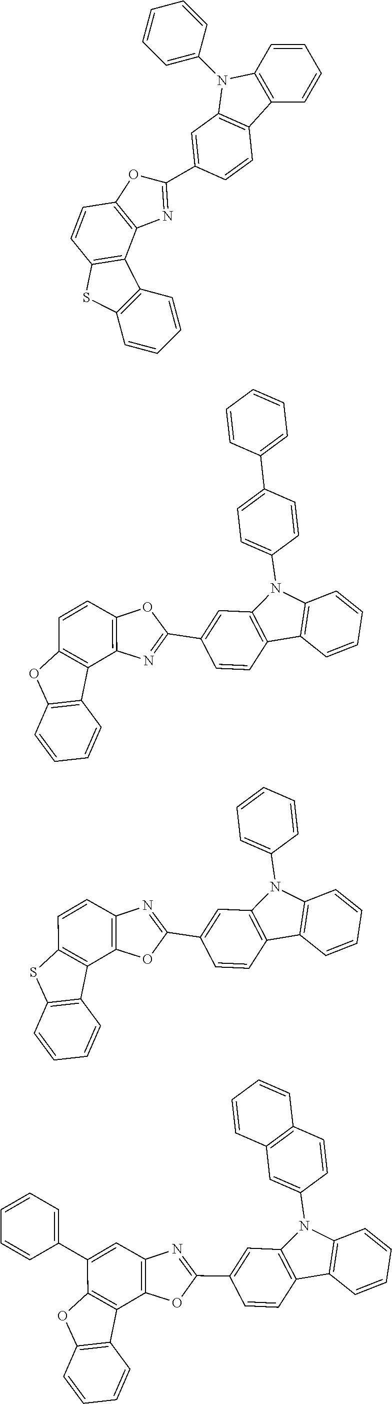 [SMILES: c1ccc(-n2c3ccccc3c3ccc(-c4nc5c(ccc6sc7ccccc7c65)o4)cc32)cc1 ; c1ccc(-c2ccc(-n3c4ccccc4c4ccc(-c5nc6c(ccc7oc8ccccc8c76)o5)cc43)cc2)cc1 ; c1ccc(-n2c3ccccc3c3ccc(-c4nc5ccc6sc7ccccc7c6c5o4)cc32)cc1 ; c1ccc(-c2cc3nc(-c4ccc5c6ccccc6n(-c6ccc7ccccc7c6)c5c4)oc3c3c2oc2ccccc23)cc1]

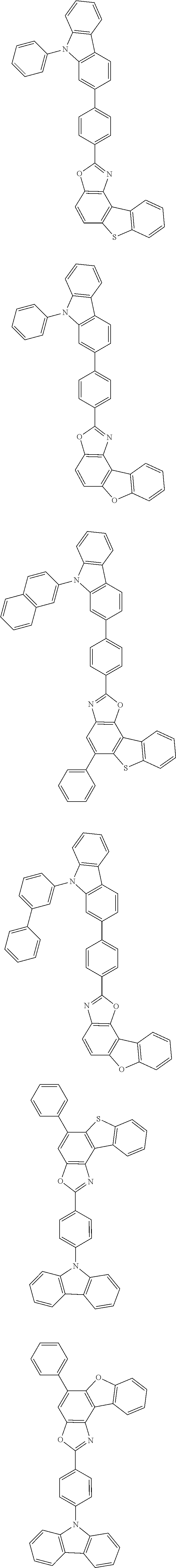 [SMILES: c1ccc(-n2c3ccccc3c3ccc(-c4ccc(-c5nc6c(ccc7sc8ccccc8c76)o5)cc4)cc32)cc1 ; c1ccc(-n2c3ccccc3c3ccc(-c4ccc(-c5nc6c(ccc7oc8ccccc8c76)o5)cc4)cc32)cc1 ; c1ccc(-c2cc3nc(-c4ccc(-c5ccc6c7ccccc7n(-c7ccc8ccccc8c7)c6c5)cc4)oc3c3c2sc2ccccc23)cc1 ; c1ccc(-c2cccc(-n3c4ccccc4c4ccc(-c5ccc(-c6nc7ccc8oc9ccccc9c8c7o6)cc5)cc43)c2)cc1 ; c1ccc(-c2cc3oc(-c4ccc(-n5c6ccccc6c6ccccc65)cc4)nc3c3c2sc2ccccc23)cc1 ; c1ccc(-c2cc3oc(-c4ccc(-n5c6ccccc6c6ccccc65)cc4)nc3c3c2oc2ccccc23)cc1]

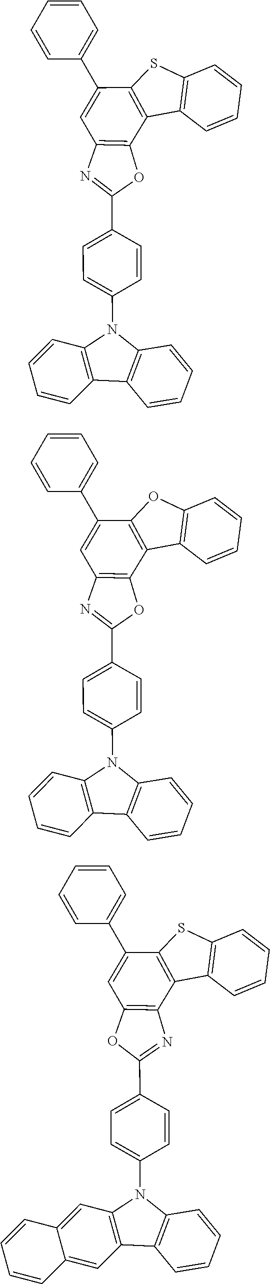

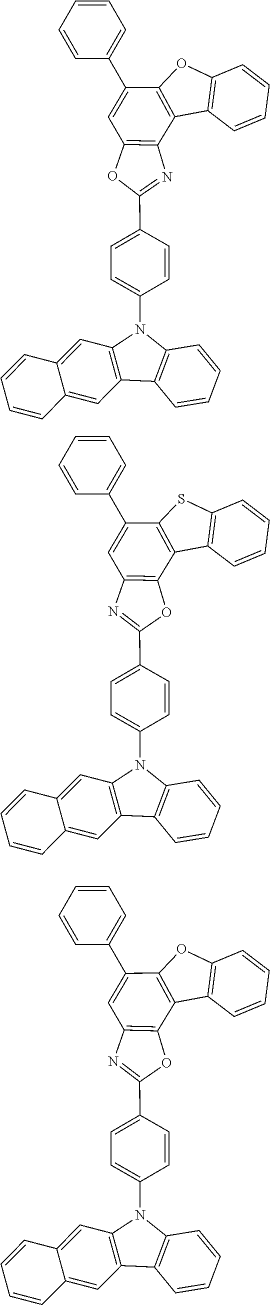

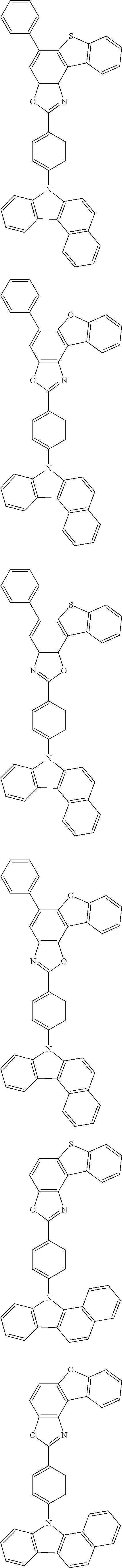

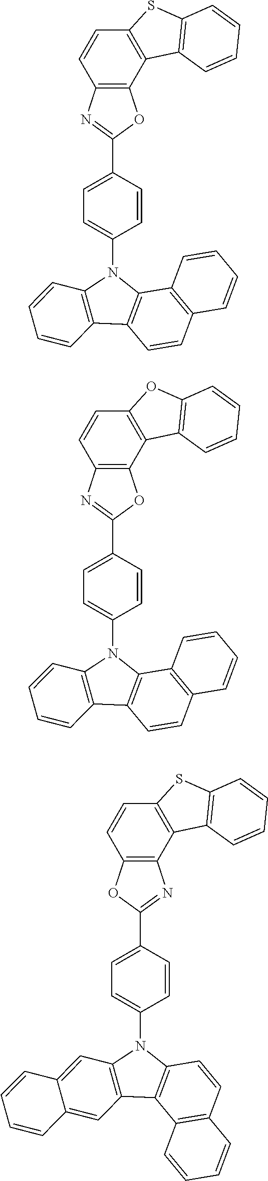

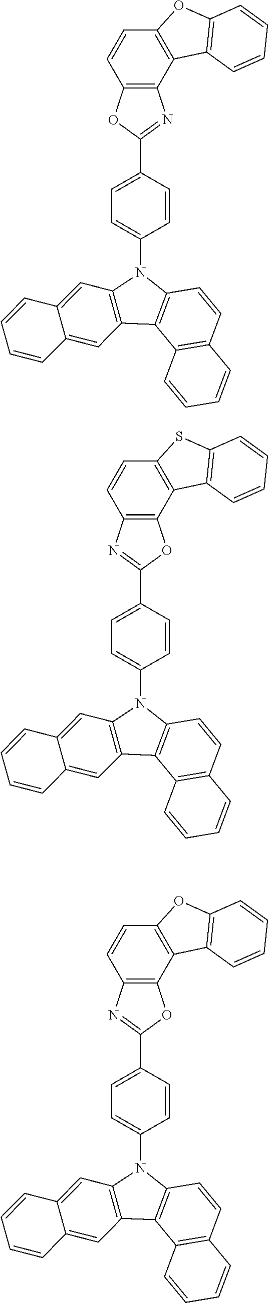

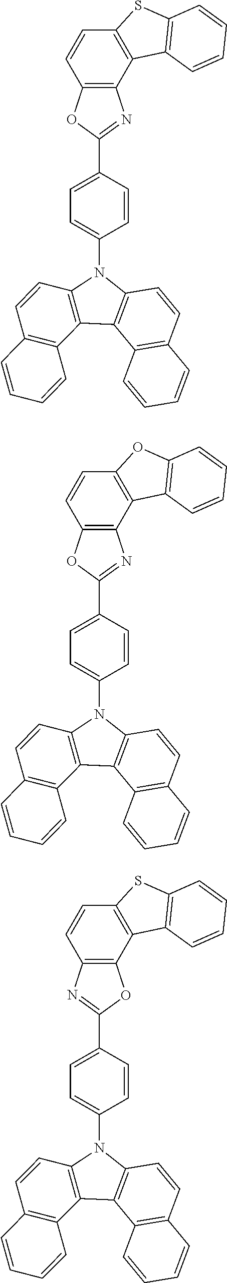

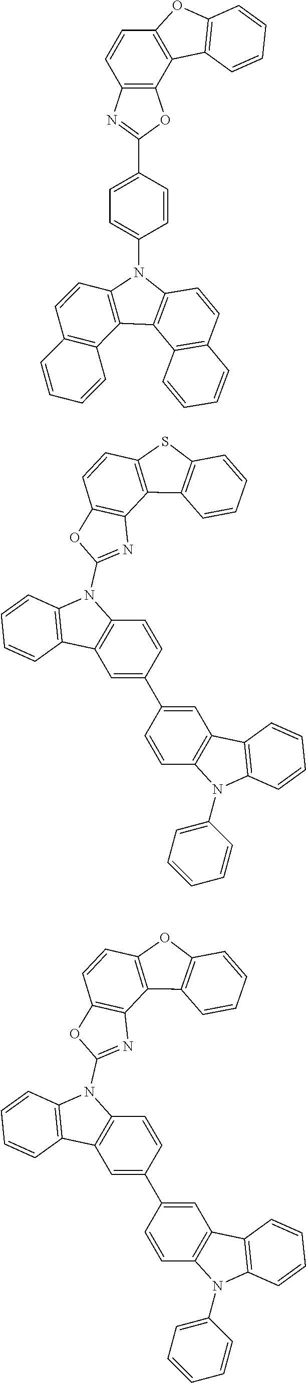

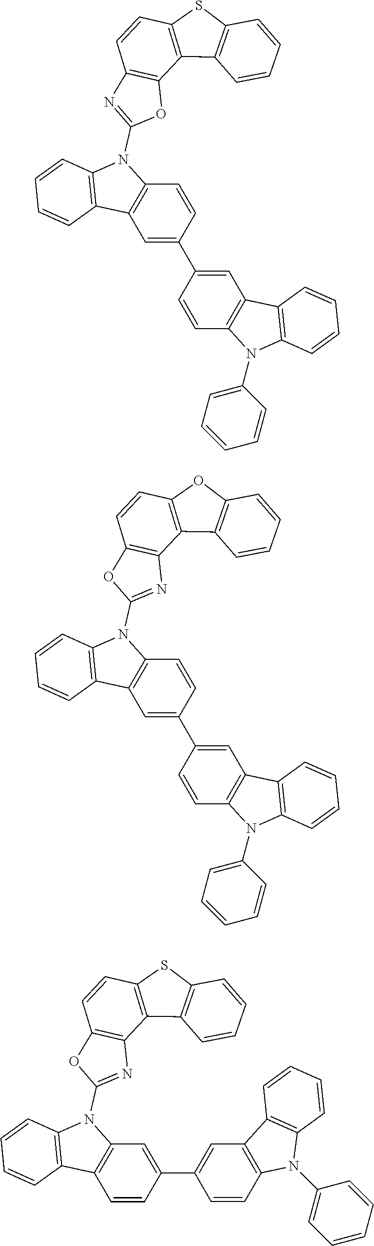

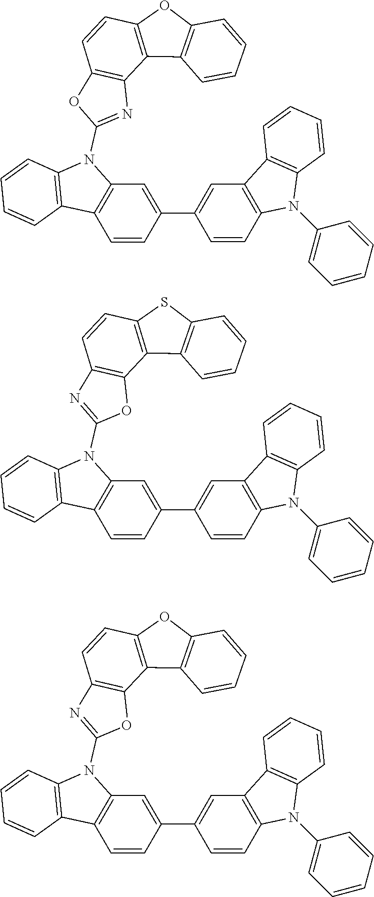 [SMILES: c1ccc(-n2c3ccccc3c3cc(-c4ccc5c6ccccc6n(-c6nc7c(ccc8oc9ccccc9c87)o6)c5c4)ccc32)cc1 ; c1ccc(-n2c3ccccc3c3cc(-c4ccc5c6ccccc6n(-c6nc7ccc8sc9ccccc9c8c7o6)c5c4)ccc32)cc1 ; c1ccc(-n2c3ccccc3c3cc(-c4ccc5c6ccccc6n(-c6nc7ccc8oc9ccccc9c8c7o6)c5c4)ccc32)cc1]

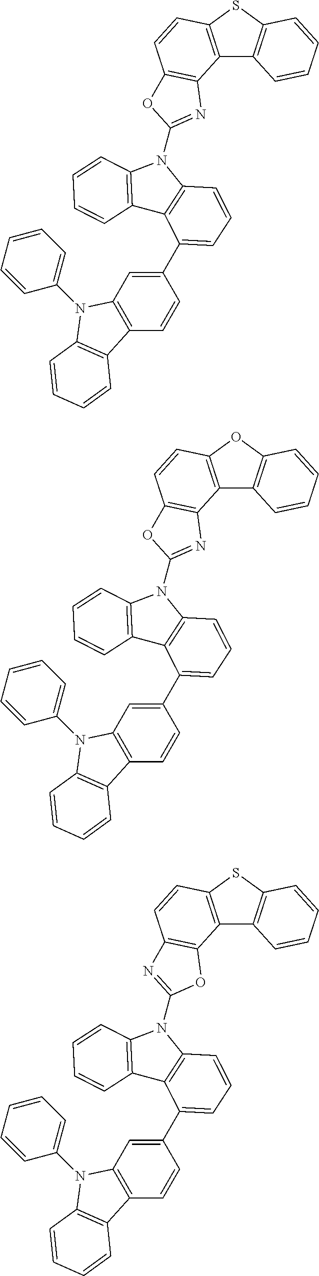 [SMILES: c1ccc(-n2c3ccccc3c3ccc(-c4cccc5c4c4ccccc4n5-c4nc5c(ccc6sc7ccccc7c65)o4)cc32)cc1 ; c1ccc(-n2c3ccccc3c3ccc(-c4cccc5c4c4ccccc4n5-c4nc5c(ccc6oc7ccccc7c65)o4)cc32)cc1 ; c1ccc(-n2c3ccccc3c3ccc(-c4cccc5c4c4ccccc4n5-c4nc5ccc6sc7ccccc7c6c5o4)cc32)cc1]

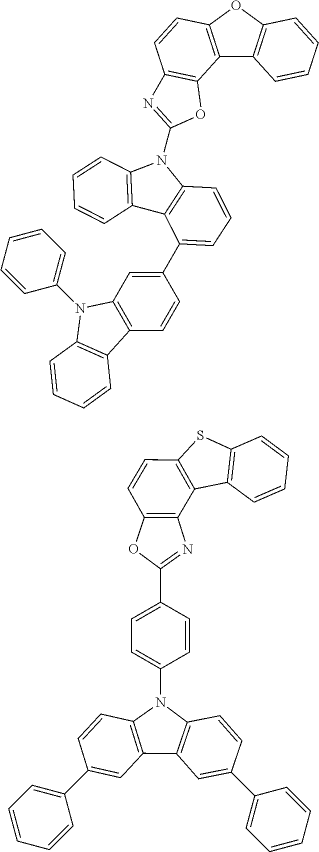 [SMILES: c1ccc(-n2c3ccccc3c3ccc(-c4cccc5c4c4ccccc4n5-c4nc5ccc6oc7ccccc7c6c5o4)cc32)cc1 ; c1ccc(-c2ccc3c(c2)c2cc(-c4ccccc4)ccc2n3-c2ccc(-c3nc4c(ccc5sc6ccccc6c54)o3)cc2)cc1]

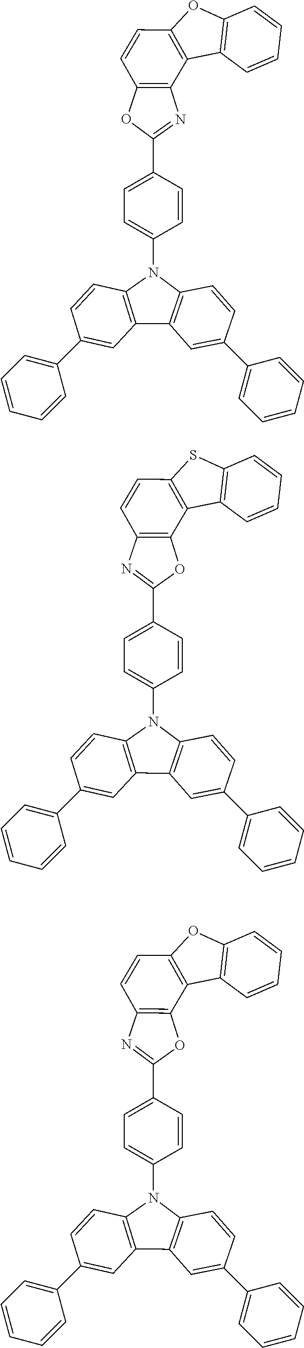

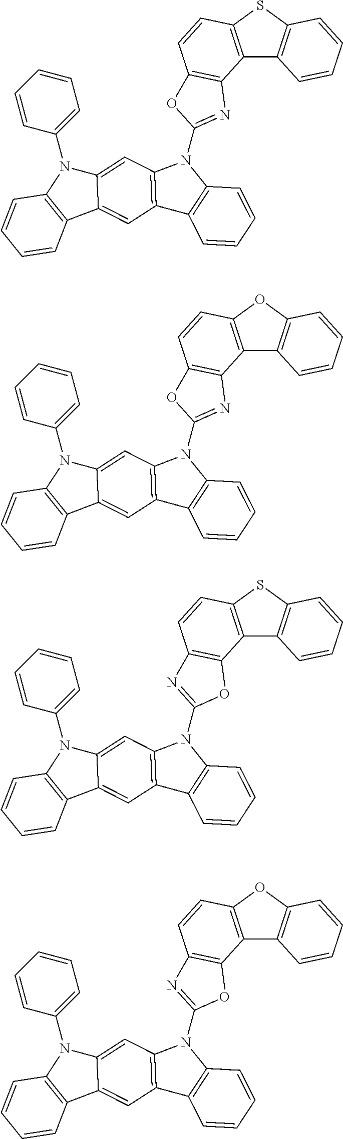 [SMILES: c1ccc(-n2c3ccccc3c3cc4c5ccccc5n(-c5nc6c(ccc7sc8ccccc8c76)o5)c4cc32)cc1 ; c1ccc(-n2c3ccccc3c3cc4c5ccccc5n(-c5nc6c(ccc7oc8ccccc8c76)o5)c4cc32)cc1 ; c1ccc(-n2c3ccccc3c3cc4c5ccccc5n(-c5nc6ccc7sc8ccccc8c7c6o5)c4cc32)cc1 ; c1ccc(-n2c3ccccc3c3cc4c5ccccc5n(-c5nc6ccc7oc8ccccc8c7c6o5)c4cc32)cc1]

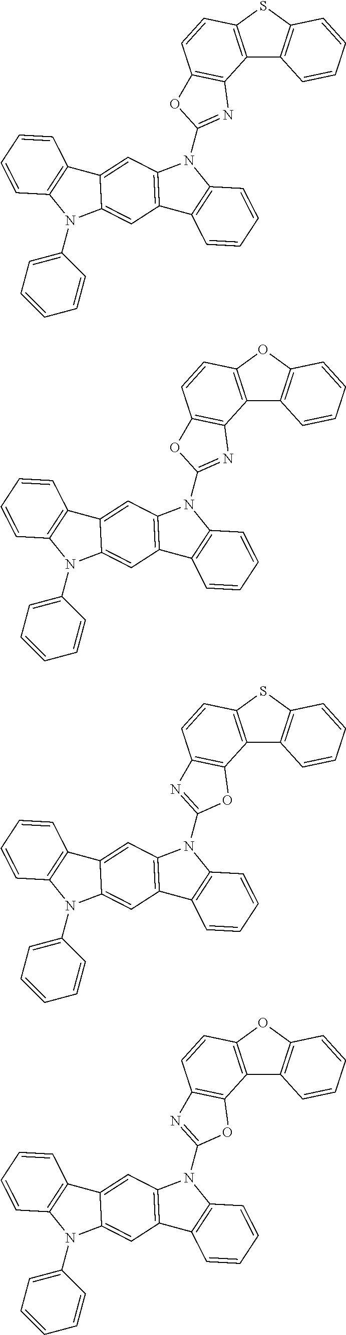 [SMILES: c1ccc(-n2c3ccccc3c3cc4c(cc32)c2ccccc2n4-c2nc3c(ccc4sc5ccccc5c43)o2)cc1 ; c1ccc(-n2c3ccccc3c3cc4c(cc32)c2ccccc2n4-c2nc3c(ccc4oc5ccccc5c43)o2)cc1 ; c1ccc(-n2c3ccccc3c3cc4c(cc32)c2ccccc2n4-c2nc3ccc4sc5ccccc5c4c3o2)cc1 ; c1ccc(-n2c3ccccc3c3cc4c(cc32)c2ccccc2n4-c2nc3ccc4oc5ccccc5c4c3o2)cc1]

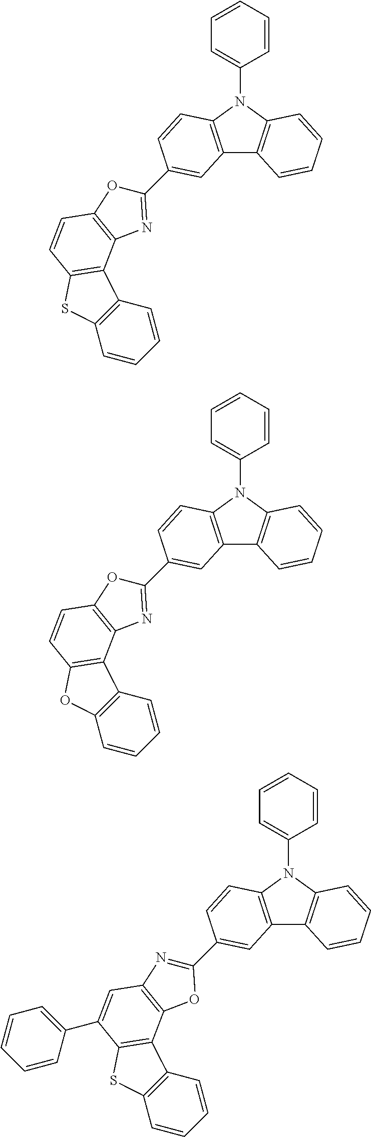 [SMILES: c1ccc(-n2c3ccccc3c3cc(-c4nc5c(ccc6sc7ccccc7c65)o4)ccc32)cc1 ; c1ccc(-n2c3ccccc3c3cc(-c4nc5c(ccc6oc7ccccc7c65)o4)ccc32)cc1 ; c1ccc(-c2cc3nc(-c4ccc5c(c4)c4ccccc4n5-c4ccccc4)oc3c3c2sc2ccccc23)cc1]

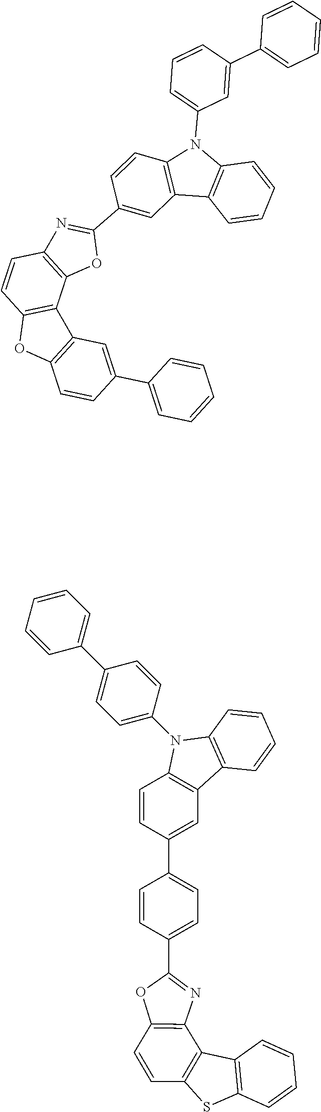 [SMILES: c1ccc(-c2cccc(-n3c4ccccc4c4cc(-c5nc6ccc7oc8ccc(-c9ccccc9)cc8c7c6o5)ccc43)c2)cc1 ; c1ccc(-c2ccc(-n3c4ccccc4c4cc(-c5ccc(-c6nc7c(ccc8sc9ccccc9c87)o6)cc5)ccc43)cc2)cc1]

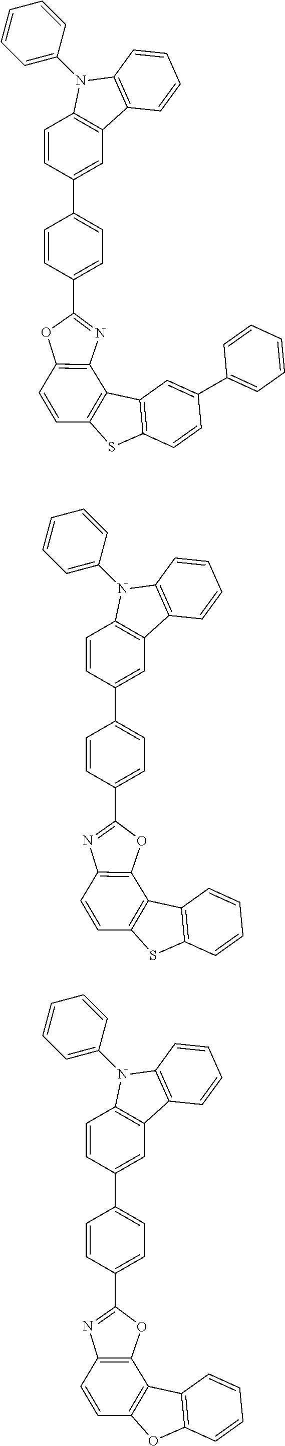 [SMILES: c1ccc(-c2ccc3sc4ccc5oc(-c6ccc(-c7ccc8c(c7)c7ccccc7n8-c7ccccc7)cc6)nc5c4c3c2)cc1 ; c1ccc(-n2c3ccccc3c3cc(-c4ccc(-c5nc6ccc7sc8ccccc8c7c6o5)cc4)ccc32)cc1 ; c1ccc(-n2c3ccccc3c3cc(-c4ccc(-c5nc6ccc7oc8ccccc8c7c6o5)cc4)ccc32)cc1]

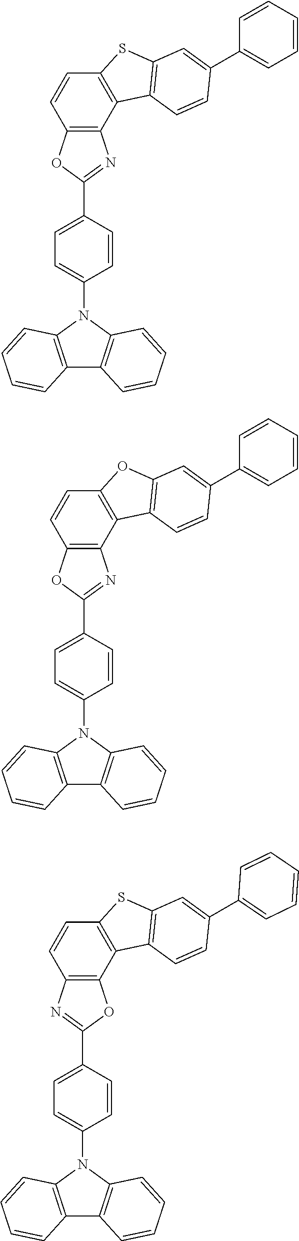 [SMILES: c1ccc(-c2ccc3c(c2)sc2ccc4oc(-c5ccc(-n6c7ccccc7c7ccccc76)cc5)nc4c23)cc1 ; c1ccc(-c2ccc3c(c2)oc2ccc4oc(-c5ccc(-n6c7ccccc7c7ccccc76)cc5)nc4c23)cc1 ; c1ccc(-c2ccc3c(c2)sc2ccc4nc(-c5ccc(-n6c7ccccc7c7ccccc76)cc5)oc4c23)cc1]

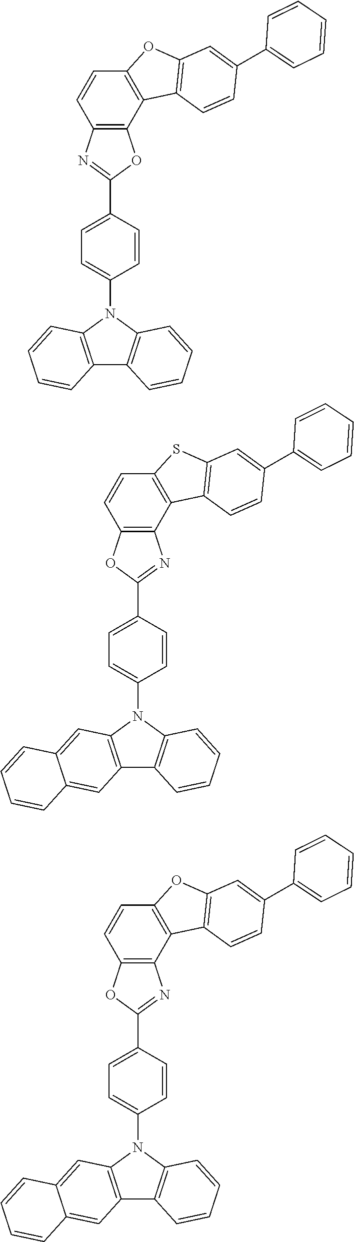 [SMILES: c1ccc(-c2ccc3c(c2)oc2ccc4nc(-c5ccc(-n6c7ccccc7c7ccccc76)cc5)oc4c23)cc1 ; c1ccc(-c2ccc3c(c2)sc2ccc4oc(-c5ccc(-n6c7ccccc7c7cc8ccccc8cc76)cc5)nc4c23)cc1 ; c1ccc(-c2ccc3c(c2)oc2ccc4oc(-c5ccc(-n6c7ccccc7c7cc8ccccc8cc76)cc5)nc4c23)cc1]

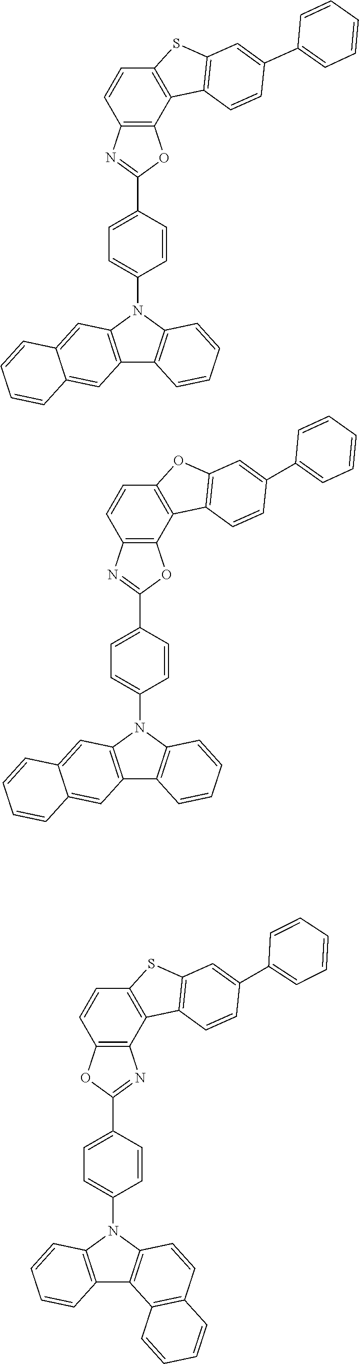 [SMILES: c1ccc(-c2ccc3c(c2)sc2ccc4nc(-c5ccc(-n6c7ccccc7c7cc8ccccc8cc76)cc5)oc4c23)cc1 ; c1ccc(-c2ccc3c(c2)oc2ccc4nc(-c5ccc(-n6c7ccccc7c7cc8ccccc8cc76)cc5)oc4c23)cc1 ; c1ccc(-c2ccc3c(c2)sc2ccc4oc(-c5ccc(-n6c7ccccc7c7c8ccccc8ccc76)cc5)nc4c23)cc1]

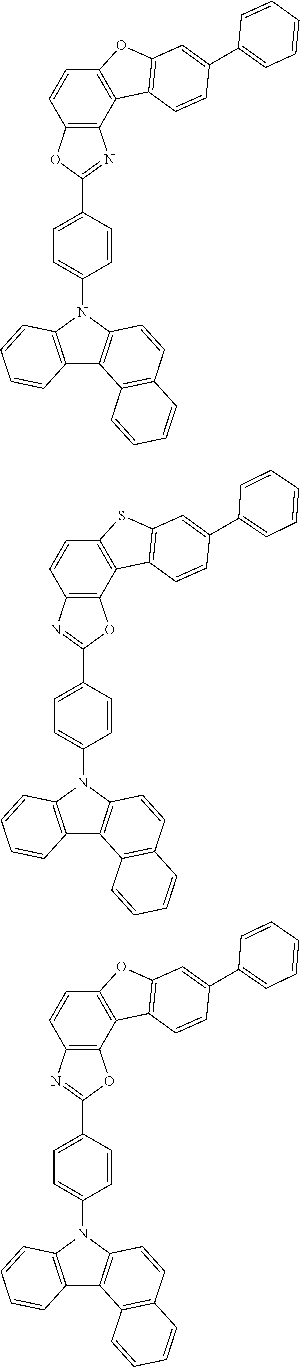 [SMILES: c1ccc(-c2ccc3c(c2)oc2ccc4oc(-c5ccc(-n6c7ccccc7c7c8ccccc8ccc76)cc5)nc4c23)cc1 ; c1ccc(-c2ccc3c(c2)sc2ccc4nc(-c5ccc(-n6c7ccccc7c7c8ccccc8ccc76)cc5)oc4c23)cc1 ; c1ccc(-c2ccc3c(c2)oc2ccc4nc(-c5ccc(-n6c7ccccc7c7c8ccccc8ccc76)cc5)oc4c23)cc1]

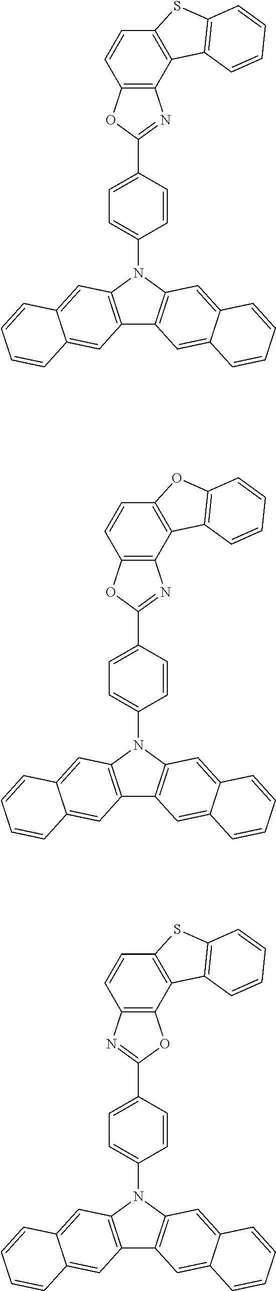 [SMILES: c1ccc2cc3c(cc2c1)c1cc2ccccc2cc1n3-c1ccc(-c2nc3c(ccc4sc5ccccc5c43)o2)cc1 ; c1ccc2cc3c(cc2c1)c1cc2ccccc2cc1n3-c1ccc(-c2nc3c(ccc4oc5ccccc5c43)o2)cc1 ; c1ccc2cc3c(cc2c1)c1cc2ccccc2cc1n3-c1ccc(-c2nc3ccc4sc5ccccc5c4c3o2)cc1]

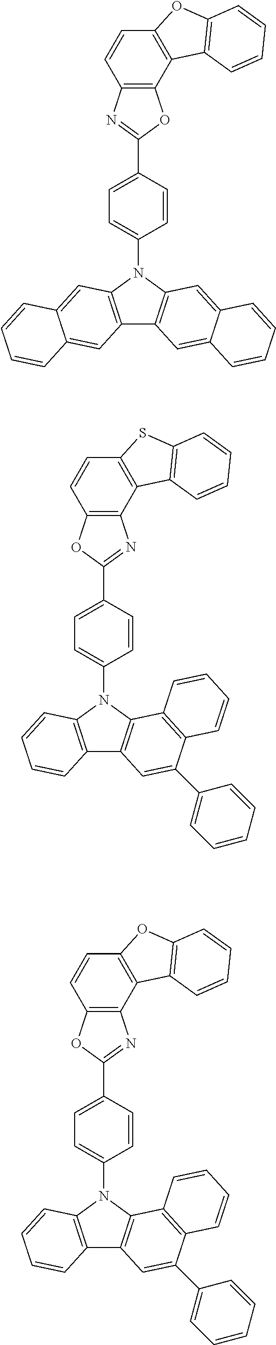 [SMILES: c1ccc2cc3c(cc2c1)c1cc2ccccc2cc1n3-c1ccc(-c2nc3ccc4oc5ccccc5c4c3o2)cc1 ; c1ccc(-c2cc3c4ccccc4n(-c4ccc(-c5nc6c(ccc7sc8ccccc8c76)o5)cc4)c3c3ccccc23)cc1 ; c1ccc(-c2cc3c4ccccc4n(-c4ccc(-c5nc6c(ccc7oc8ccccc8c76)o5)cc4)c3c3ccccc23)cc1]

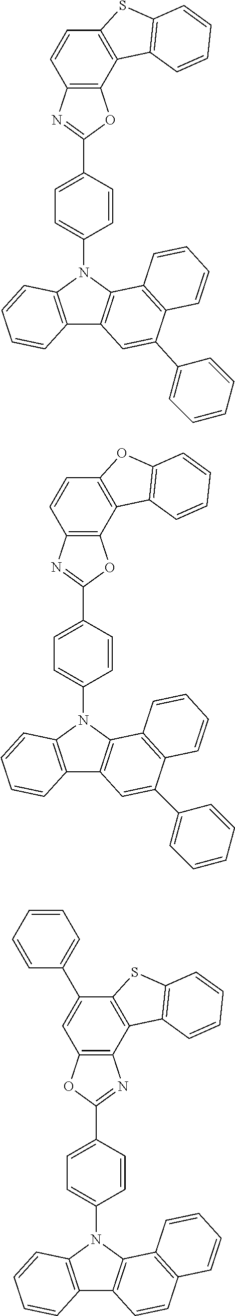

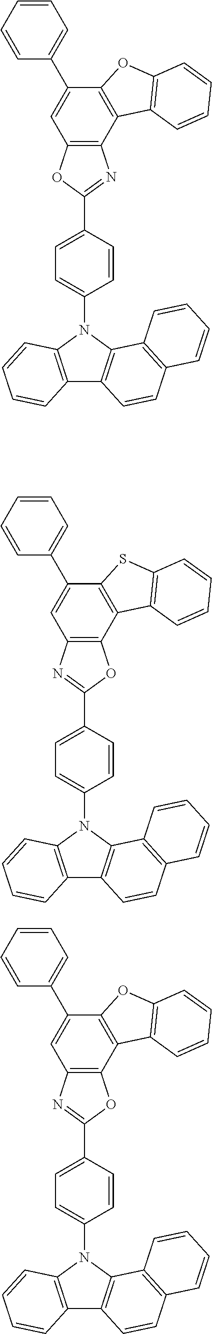

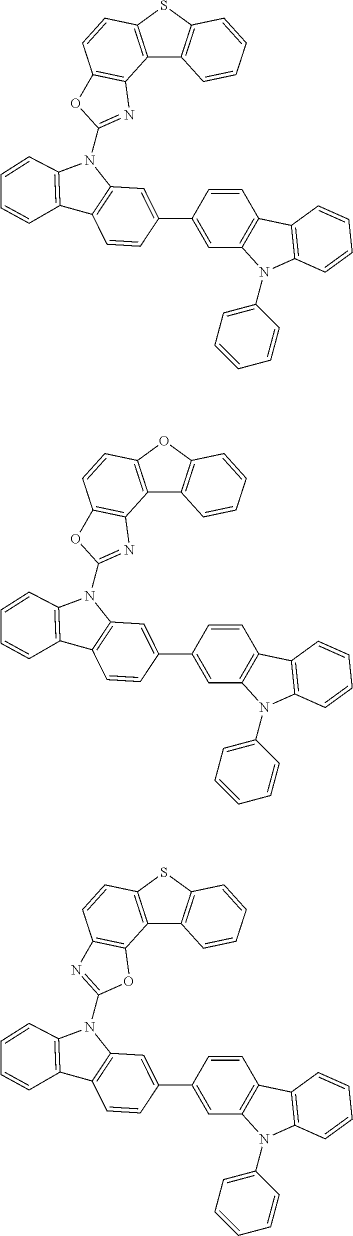 [SMILES: c1ccc(-n2c3ccccc3c3ccc(-c4ccc5c6ccccc6n(-c6nc7c(ccc8sc9ccccc9c87)o6)c5c4)cc32)cc1 ; c1ccc(-n2c3ccccc3c3ccc(-c4ccc5c6ccccc6n(-c6nc7c(ccc8oc9ccccc9c87)o6)c5c4)cc32)cc1 ; c1ccc(-n2c3ccccc3c3ccc(-c4ccc5c6ccccc6n(-c6nc7ccc8sc9ccccc9c8c7o6)c5c4)cc32)cc1]

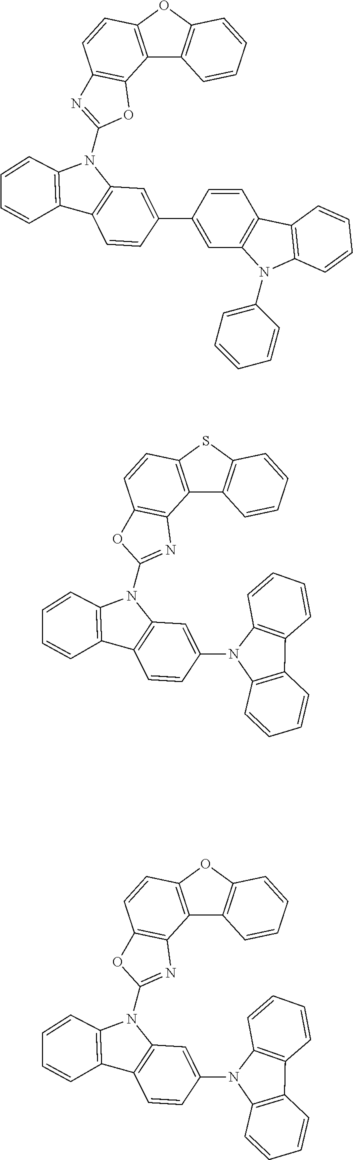 [SMILES: c1ccc(-n2c3ccccc3c3ccc(-c4ccc5c6ccccc6n(-c6nc7ccc8oc9ccccc9c8c7o6)c5c4)cc32)cc1 ; c1ccc2c(c1)sc1ccc3oc(-n4c5ccccc5c5ccc(-n6c7ccccc7c7ccccc76)cc54)nc3c12 ; c1ccc2c(c1)oc1ccc3oc(-n4c5ccccc5c5ccc(-n6c7ccccc7c7ccccc76)cc54)nc3c12]

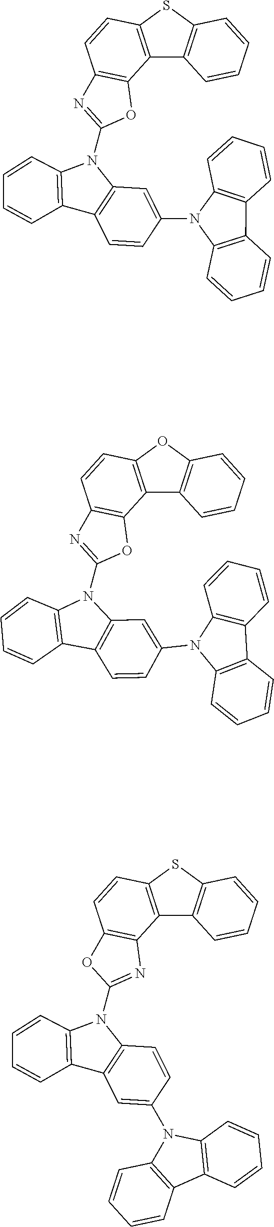 [SMILES: c1ccc2c(c1)sc1ccc3nc(-n4c5ccccc5c5ccc(-n6c7ccccc7c7ccccc76)cc54)oc3c12 ; c1ccc2c(c1)oc1ccc3nc(-n4c5ccccc5c5ccc(-n6c7ccccc7c7ccccc76)cc54)oc3c12 ; c1ccc2c(c1)sc1ccc3oc(-n4c5ccccc5c5cc(-n6c7ccccc7c7ccccc76)ccc54)nc3c12]

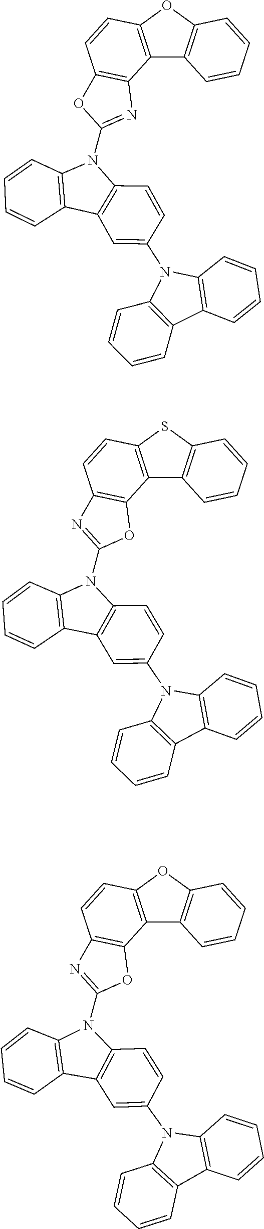 [SMILES: c1ccc2c(c1)oc1ccc3oc(-n4c5ccccc5c5cc(-n6c7ccccc7c7ccccc76)ccc54)nc3c12 ; c1ccc2c(c1)sc1ccc3nc(-n4c5ccccc5c5cc(-n6c7ccccc7c7ccccc76)ccc54)oc3c12 ; c1ccc2c(c1)oc1ccc3nc(-n4c5ccccc5c5cc(-n6c7ccccc7c7ccccc76)ccc54)oc3c12]

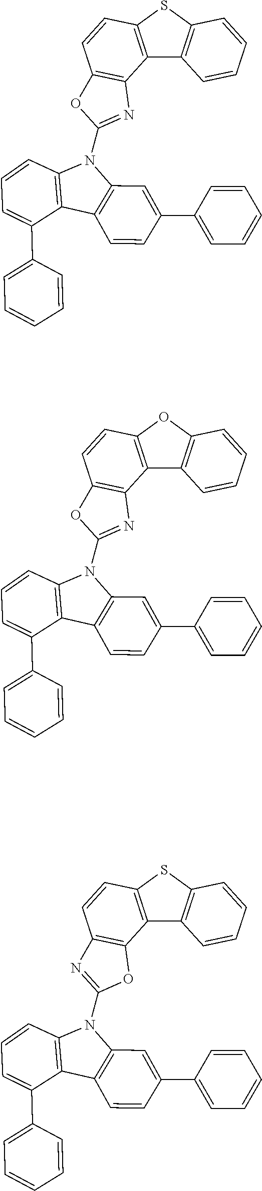 [SMILES: c1ccc(-c2ccc3c4c(-c5ccccc5)cccc4n(-c4nc5c(ccc6sc7ccccc7c65)o4)c3c2)cc1 ; c1ccc(-c2ccc3c4c(-c5ccccc5)cccc4n(-c4nc5c(ccc6oc7ccccc7c65)o4)c3c2)cc1 ; c1ccc(-c2ccc3c4c(-c5ccccc5)cccc4n(-c4nc5ccc6sc7ccccc7c6c5o4)c3c2)cc1]

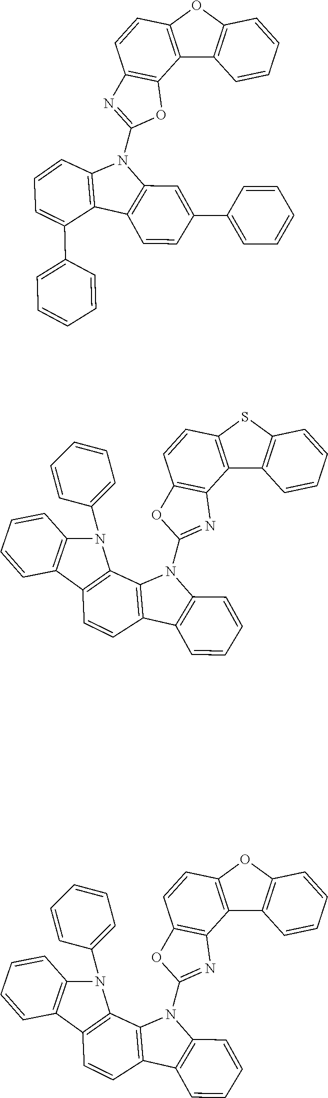 [SMILES: c1ccc(-c2ccc3c4c(-c5ccccc5)cccc4n(-c4nc5ccc6oc7ccccc7c6c5o4)c3c2)cc1 ; c1ccc(-n2c3ccccc3c3ccc4c5ccccc5n(-c5nc6c(ccc7sc8ccccc8c76)o5)c4c32)cc1 ; c1ccc(-n2c3ccccc3c3ccc4c5ccccc5n(-c5nc6c(ccc7oc8ccccc8c76)o5)c4c32)cc1]

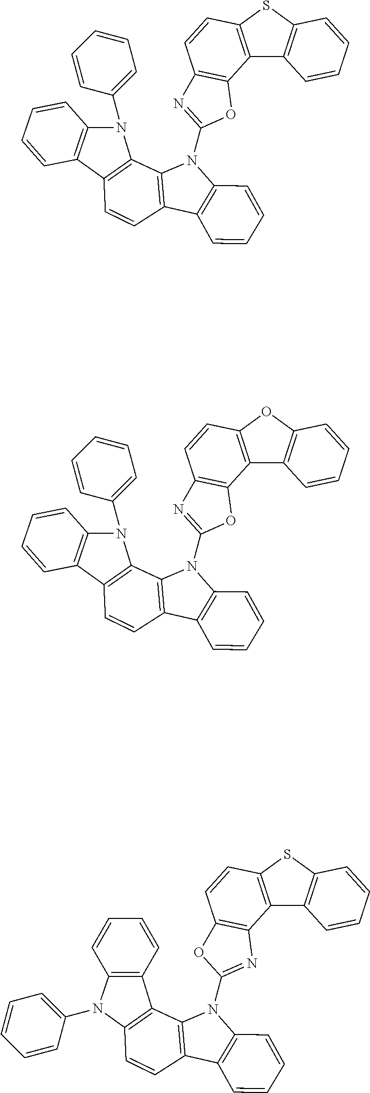 [SMILES: c1ccc(-n2c3ccccc3c3ccc4c5ccccc5n(-c5nc6ccc7sc8ccccc8c7c6o5)c4c32)cc1 ; c1ccc(-n2c3ccccc3c3ccc4c5ccccc5n(-c5nc6ccc7oc8ccccc8c7c6o5)c4c32)cc1 ; c1ccc(-n2c3ccccc3c3c2ccc2c4ccccc4n(-c4nc5c(ccc6sc7ccccc7c65)o4)c23)cc1]

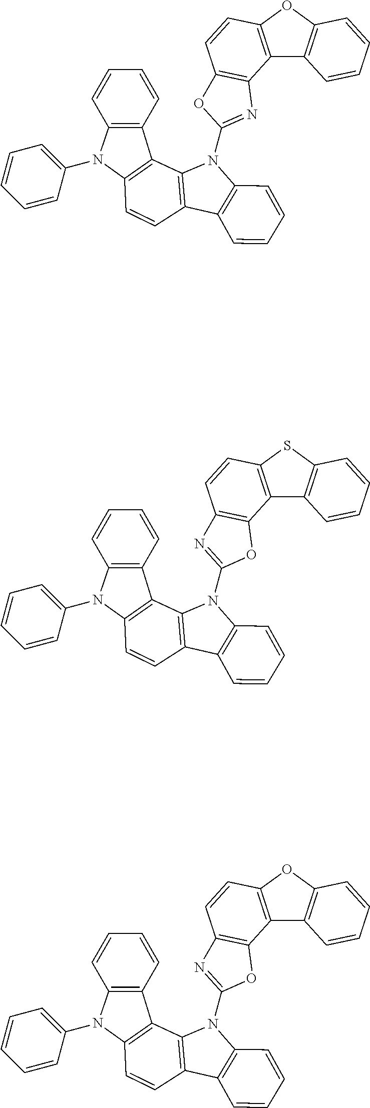 [SMILES: c1ccc(-n2c3ccccc3c3c2ccc2c4ccccc4n(-c4nc5c(ccc6oc7ccccc7c65)o4)c23)cc1 ; c1ccc(-n2c3ccccc3c3c2ccc2c4ccccc4n(-c4nc5ccc6sc7ccccc7c6c5o4)c23)cc1 ; c1ccc(-n2c3ccccc3c3c2ccc2c4ccccc4n(-c4nc5ccc6oc7ccccc7c6c5o4)c23)cc1]

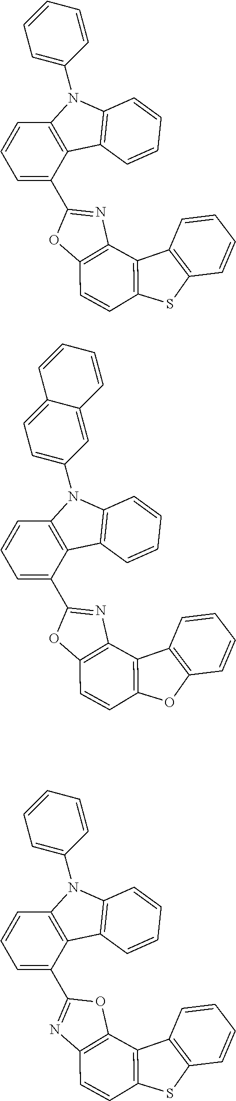

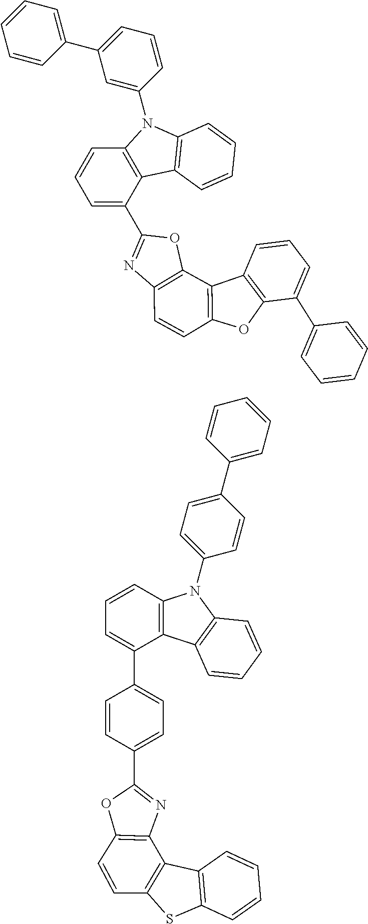 [SMILES: c1ccc(-c2cccc(-n3c4ccccc4c4c(-c5nc6ccc7oc8c(-c9ccccc9)cccc8c7c6o5)cccc43)c2)cc1 ; c1ccc(-c2ccc(-n3c4ccccc4c4c(-c5ccc(-c6nc7c(ccc8sc9ccccc9c87)o6)cc5)cccc43)cc2)cc1]

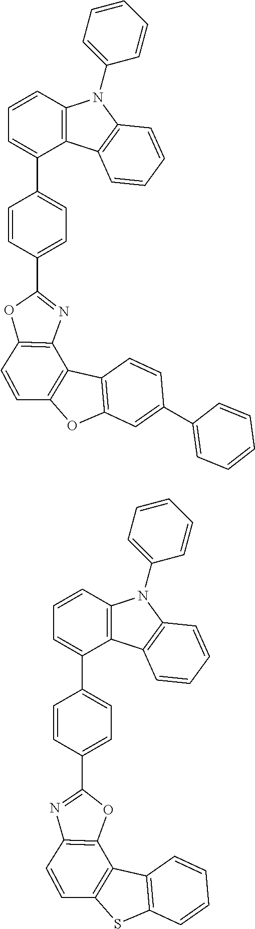 [SMILES: c1ccc(-c2ccc3c(c2)oc2ccc4oc(-c5ccc(-c6cccc7c6c6ccccc6n7-c6ccccc6)cc5)nc4c23)cc1 ; c1ccc(-n2c3ccccc3c3c(-c4ccc(-c5nc6ccc7sc8ccccc8c7c6o5)cc4)cccc32)cc1]

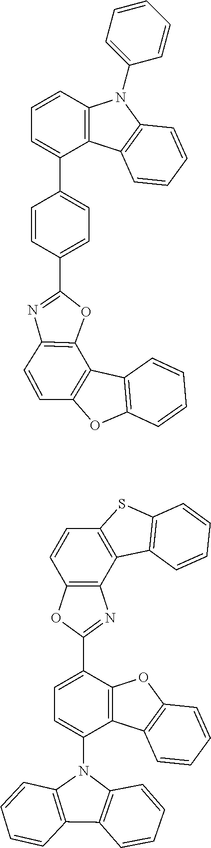 [SMILES: c1ccc(-n2c3ccccc3c3c(-c4ccc(-c5nc6ccc7oc8ccccc8c7c6o5)cc4)cccc32)cc1 ; c1ccc2c(c1)oc1c(-c3nc4c(ccc5sc6ccccc6c54)o3)ccc(-n3c4ccccc4c4ccccc43)c12]

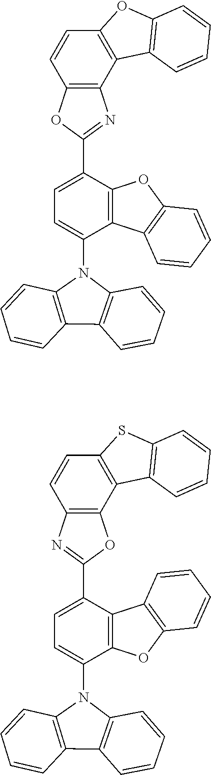 [SMILES: c1ccc2c(c1)oc1c(-c3nc4c(ccc5oc6ccccc6c54)o3)ccc(-n3c4ccccc4c4ccccc43)c12 ; c1ccc2c(c1)oc1c(-n3c4ccccc4c4ccccc43)ccc(-c3nc4ccc5sc6ccccc6c5c4o3)c12]

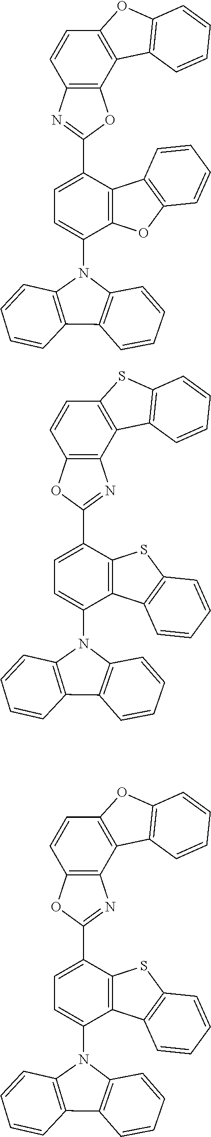

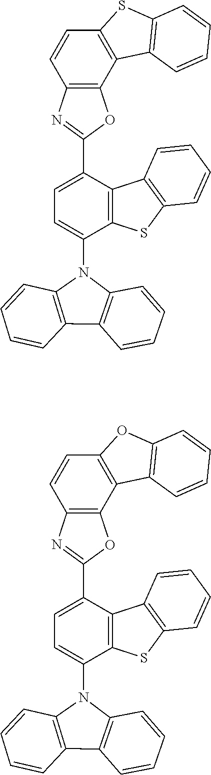 [SMILES: c1ccc2c(c1)sc1c(-n3c4ccccc4c4ccccc43)ccc(-c3nc4ccc5sc6ccccc6c5c4o3)c12 ; c1ccc2c(c1)oc1ccc3nc(-c4ccc(-n5c6ccccc6c6ccccc65)c5sc6ccccc6c45)oc3c12]

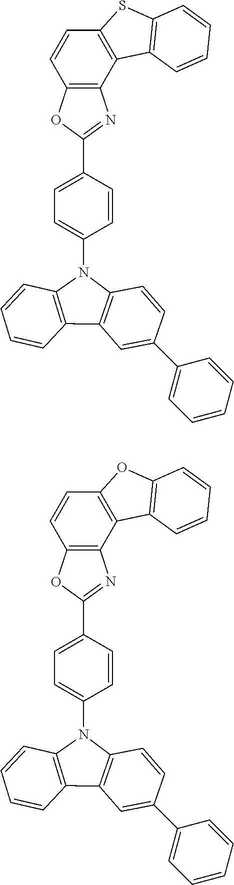

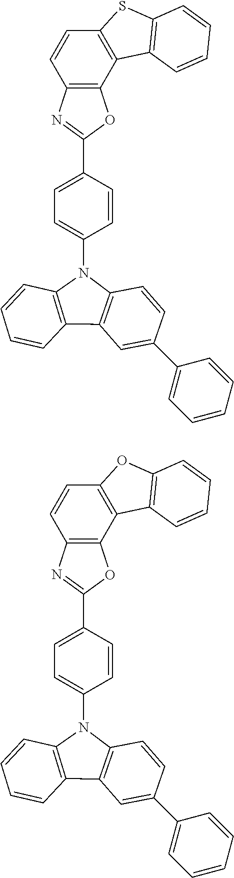

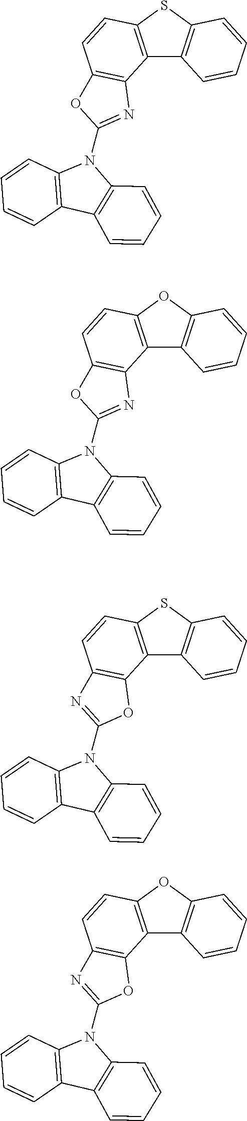

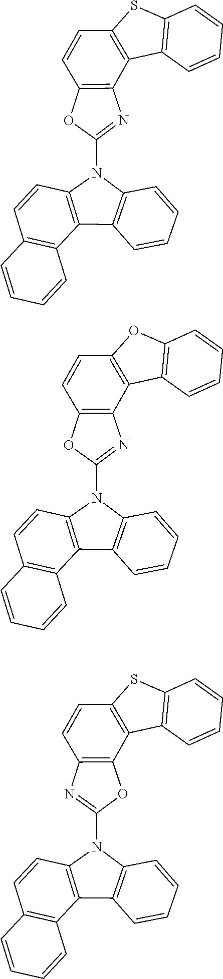 [SMILES: c1ccc2c(c1)ccc1c2c2ccccc2n1-c1nc2c(ccc3sc4ccccc4c32)o1 ; c1ccc2c(c1)ccc1c2c2ccccc2n1-c1nc2c(ccc3oc4ccccc4c32)o1 ; c1ccc2c(c1)ccc1c2c2ccccc2n1-c1nc2ccc3sc4ccccc4c3c2o1]

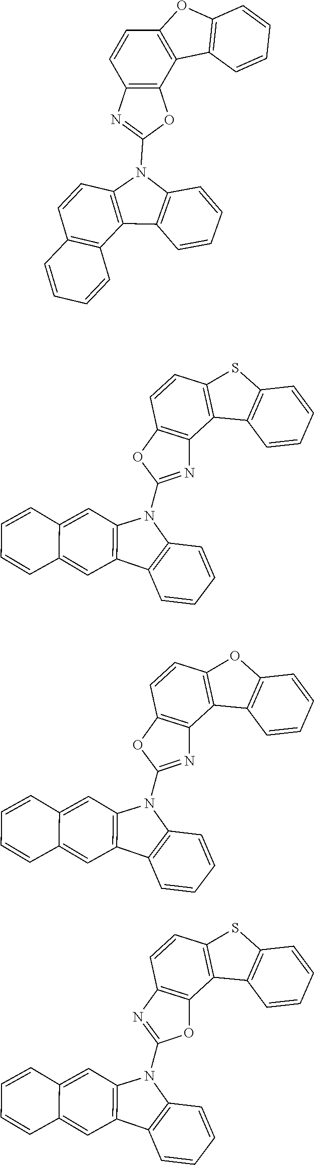 [SMILES: c1ccc2c(c1)ccc1c2c2ccccc2n1-c1nc2ccc3oc4ccccc4c3c2o1 ; c1ccc2cc3c(cc2c1)c1ccccc1n3-c1nc2c(ccc3sc4ccccc4c32)o1 ; c1ccc2cc3c(cc2c1)c1ccccc1n3-c1nc2c(ccc3oc4ccccc4c32)o1 ; c1ccc2cc3c(cc2c1)c1ccccc1n3-c1nc2ccc3sc4ccccc4c3c2o1]

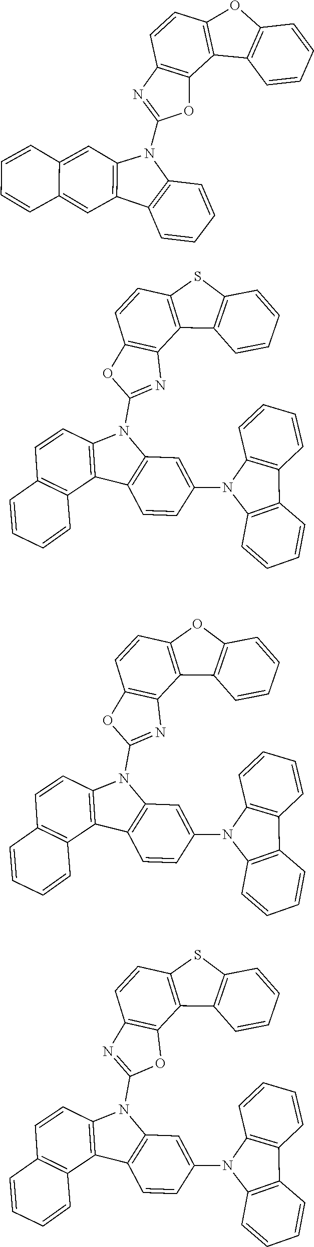

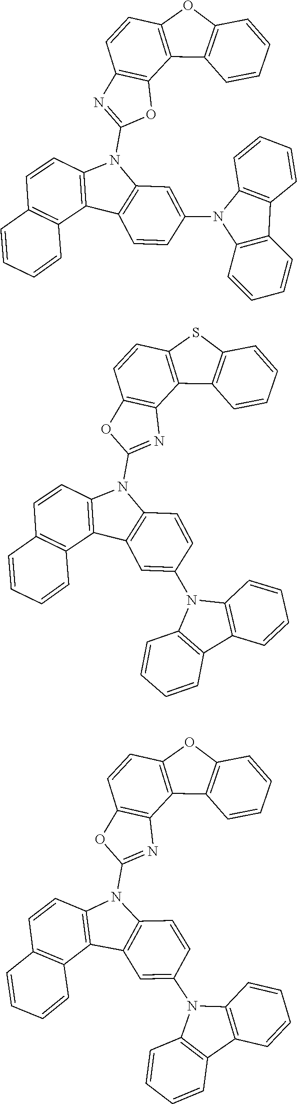

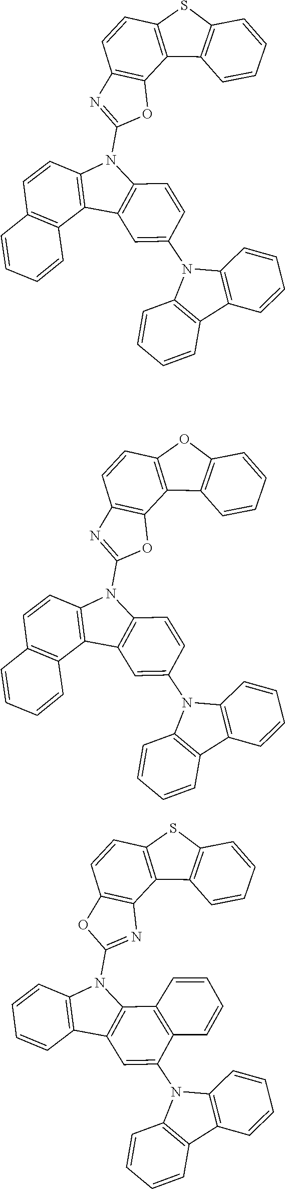

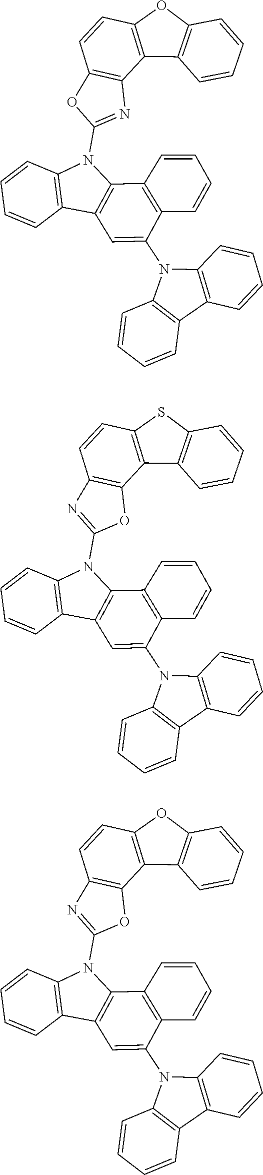

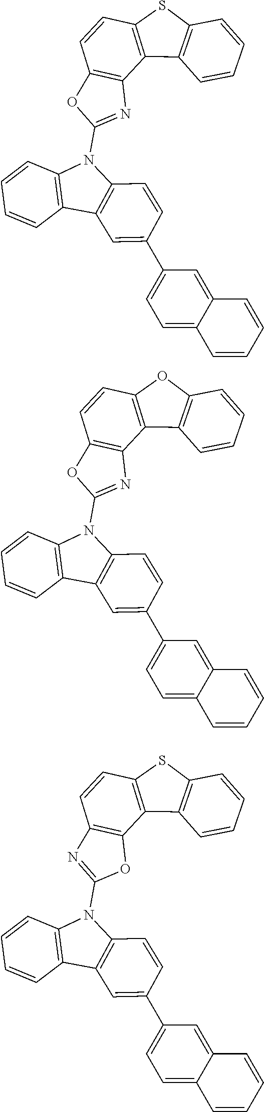

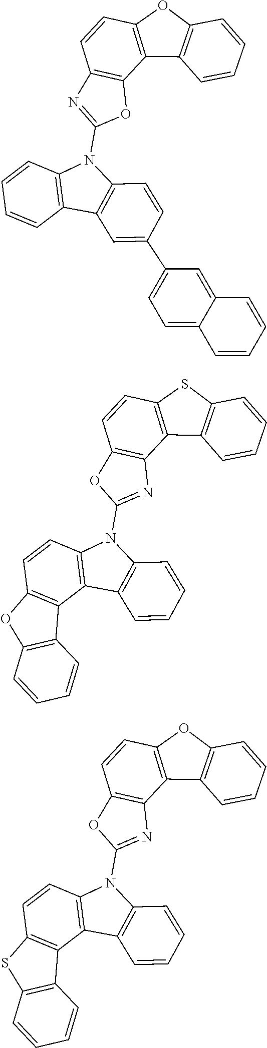

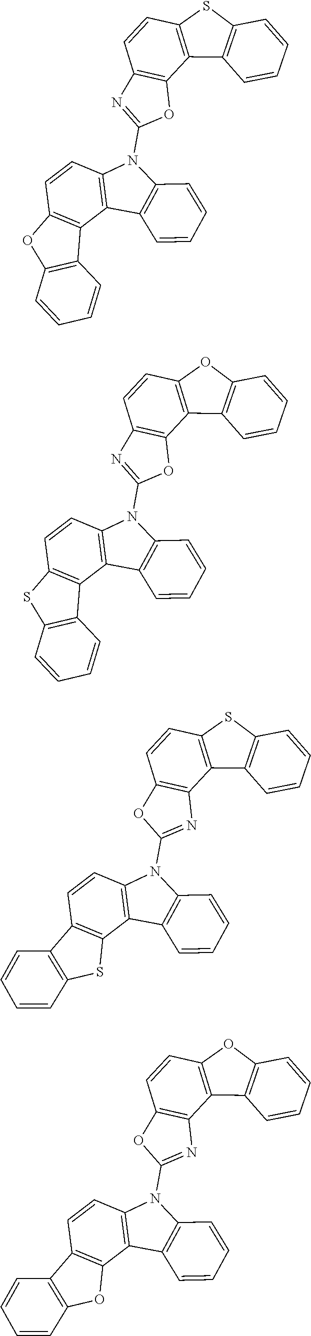 [SMILES: c1ccc2c(c1)oc1ccc3c(c4ccccc4n3-c3nc4ccc5sc6ccccc6c5c4o3)c12 ; c1ccc2c(c1)oc1ccc3nc(-n4c5ccccc5c5c6c(ccc54)sc4ccccc46)oc3c12 ; c1ccc2c(c1)sc1c2ccc2c1c1ccccc1n2-c1nc2c(ccc3sc4ccccc4c32)o1 ; c1ccc2c(c1)oc1c2ccc2c1c1ccccc1n2-c1nc2c(ccc3oc4ccccc4c32)o1]

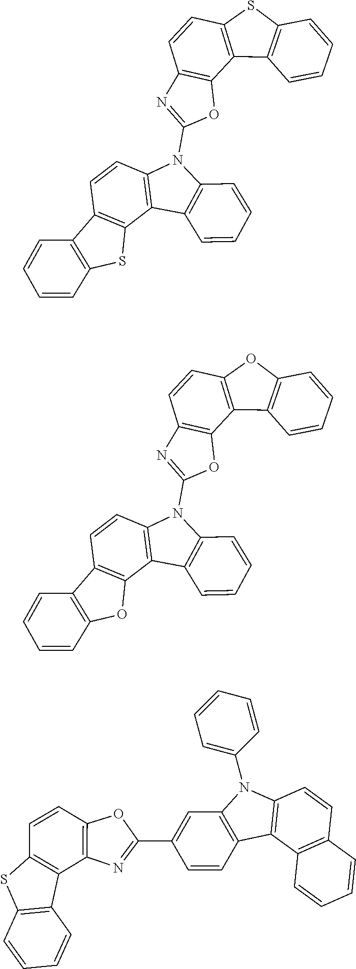 [SMILES: c1ccc2c(c1)sc1c2ccc2c1c1ccccc1n2-c1nc2ccc3sc4ccccc4c3c2o1 ; c1ccc2c(c1)oc1c2ccc2c1c1ccccc1n2-c1nc2ccc3oc4ccccc4c3c2o1 ; c1ccc(-n2c3cc(-c4nc5c(ccc6sc7ccccc7c65)o4)ccc3c3c4ccccc4ccc32)cc1]

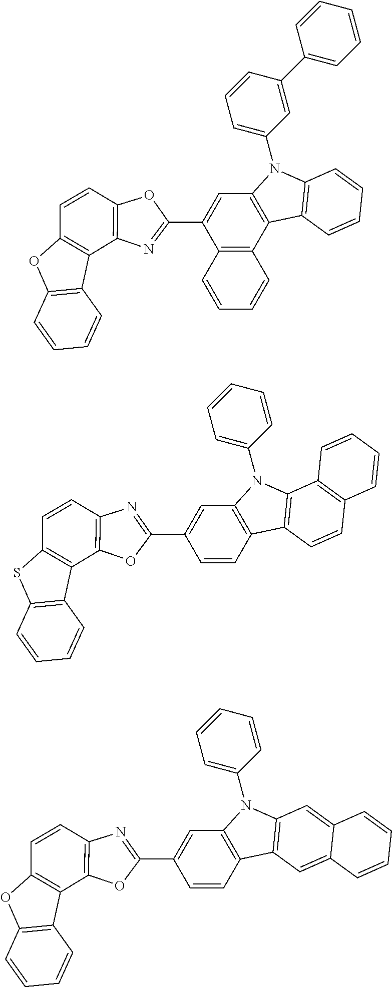 [SMILES: c1ccc(-c2cccc(-n3c4ccccc4c4c5ccccc5c(-c5nc6c(ccc7oc8ccccc8c76)o5)cc43)c2)cc1 ; c1ccc(-n2c3cc(-c4nc5ccc6sc7ccccc7c6c5o4)ccc3c3ccc4ccccc4c32)cc1 ; c1ccc(-n2c3cc(-c4nc5ccc6oc7ccccc7c6c5o4)ccc3c3cc4ccccc4cc32)cc1]

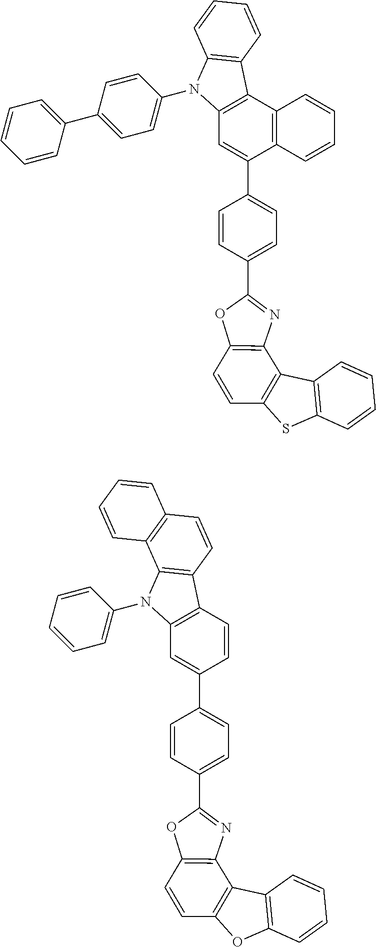 [SMILES: c1ccc(-c2ccc(-n3c4ccccc4c4c5ccccc5c(-c5ccc(-c6nc7c(ccc8sc9ccccc9c87)o6)cc5)cc43)cc2)cc1 ; c1ccc(-n2c3cc(-c4ccc(-c5nc6c(ccc7oc8ccccc8c76)o5)cc4)ccc3c3ccc4ccccc4c32)cc1]

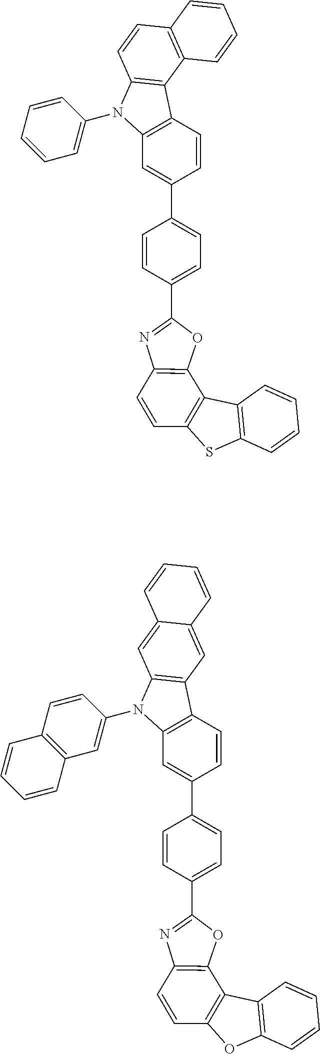 [SMILES: c1ccc(-n2c3cc(-c4ccc(-c5nc6ccc7sc8ccccc8c7c6o5)cc4)ccc3c3c4ccccc4ccc32)cc1 ; c1ccc2cc(-n3c4cc(-c5ccc(-c6nc7ccc8oc9ccccc9c8c7o6)cc5)ccc4c4cc5ccccc5cc43)ccc2c1]

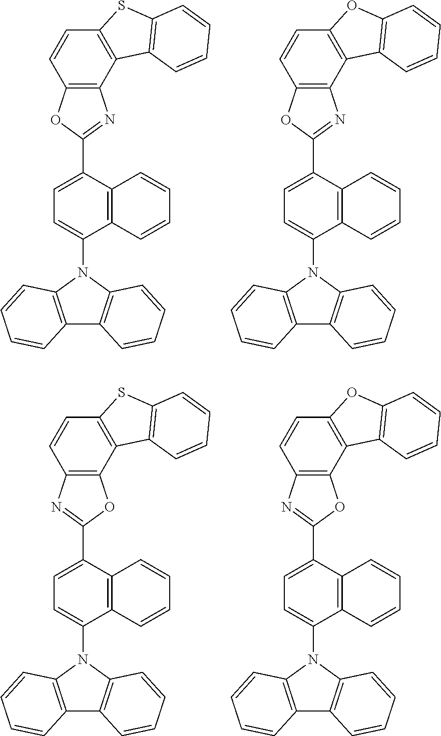

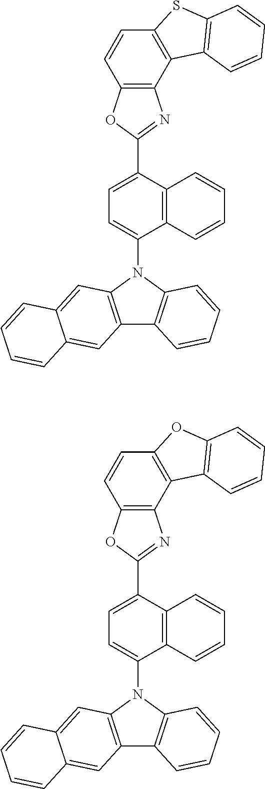 [SMILES: c1ccc2cc3c(cc2c1)c1ccccc1n3-c1ccc(-c2nc3c(ccc4sc5ccccc5c43)o2)c2ccccc12 ; c1ccc2cc3c(cc2c1)c1ccccc1n3-c1ccc(-c2nc3c(ccc4oc5ccccc5c43)o2)c2ccccc12]

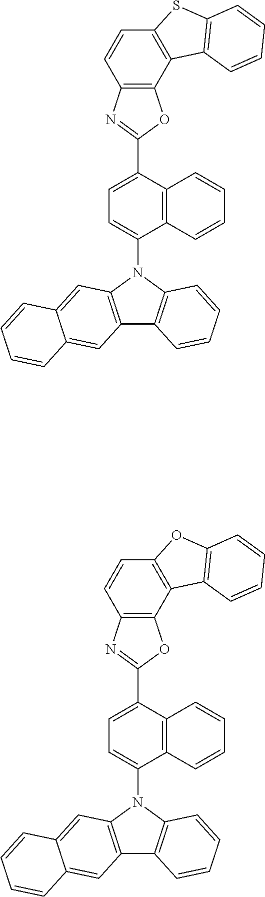 [SMILES: c1ccc2cc3c(cc2c1)c1ccccc1n3-c1ccc(-c2nc3ccc4sc5ccccc5c4c3o2)c2ccccc12 ; c1ccc2cc3c(cc2c1)c1ccccc1n3-c1ccc(-c2nc3ccc4oc5ccccc5c4c3o2)c2ccccc12]

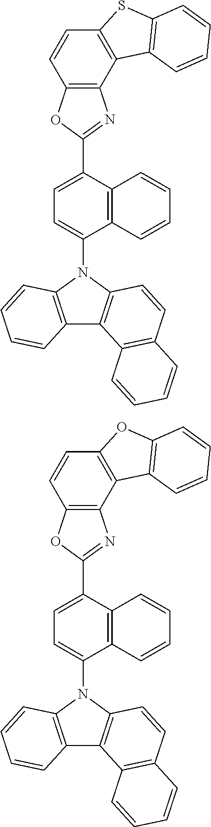

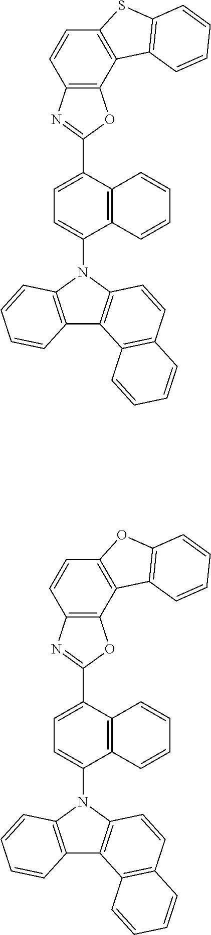 [SMILES: c1ccc2c(c1)ccc1c2c2ccccc2n1-c1ccc(-c2nc3ccc4sc5ccccc5c4c3o2)c2ccccc12 ; c1ccc2c(c1)ccc1c2c2ccccc2n1-c1ccc(-c2nc3ccc4oc5ccccc5c4c3o2)c2ccccc12]

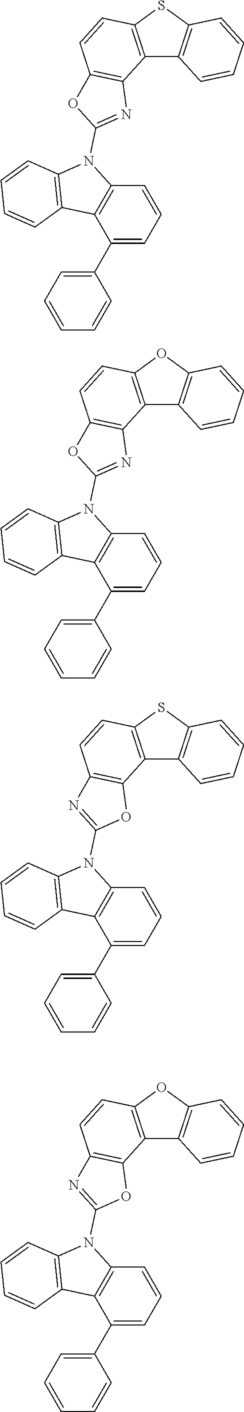

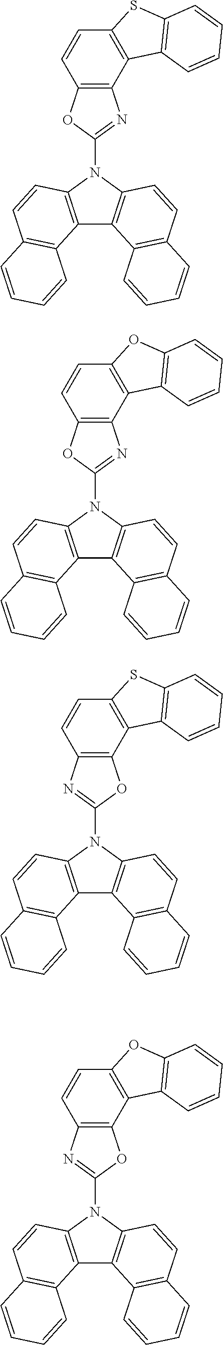

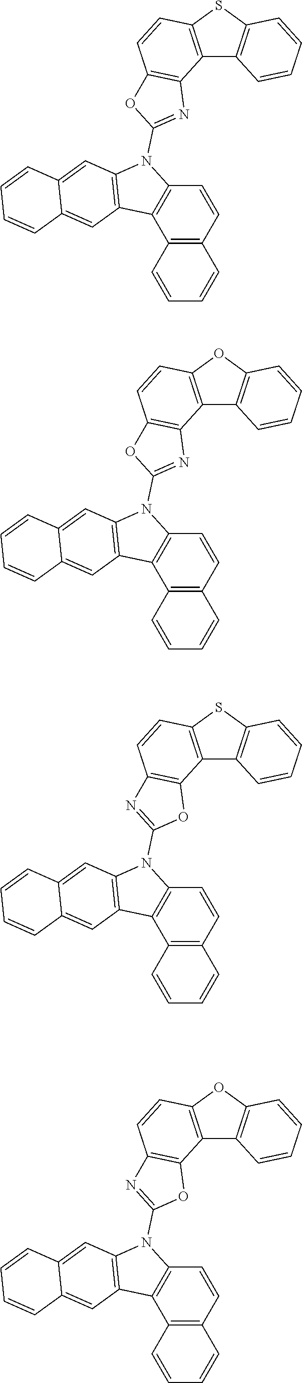 [SMILES: c1ccc2cc3c(cc2c1)c1c2ccccc2ccc1n3-c1nc2c(ccc3sc4ccccc4c32)o1 ; c1ccc2cc3c(cc2c1)c1c2ccccc2ccc1n3-c1nc2c(ccc3oc4ccccc4c32)o1 ; c1ccc2cc3c(cc2c1)c1c2ccccc2ccc1n3-c1nc2ccc3sc4ccccc4c3c2o1 ; c1ccc2cc3c(cc2c1)c1c2ccccc2ccc1n3-c1nc2ccc3oc4ccccc4c3c2o1]

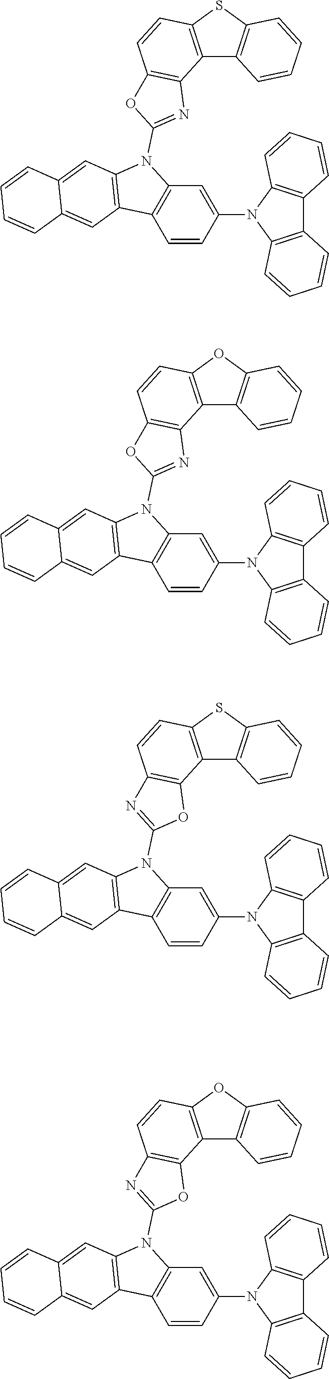 [SMILES: c1ccc2cc3c(cc2c1)c1ccc(-n2c4ccccc4c4ccccc42)cc1n3-c1nc2c(ccc3sc4ccccc4c32)o1 ; c1ccc2cc3c(cc2c1)c1ccc(-n2c4ccccc4c4ccccc42)cc1n3-c1nc2c(ccc3oc4ccccc4c32)o1 ; c1ccc2cc3c(cc2c1)c1ccc(-n2c4ccccc4c4ccccc42)cc1n3-c1nc2ccc3sc4ccccc4c3c2o1 ; c1ccc2cc3c(cc2c1)c1ccc(-n2c4ccccc4c4ccccc42)cc1n3-c1nc2ccc3oc4ccccc4c3c2o1]

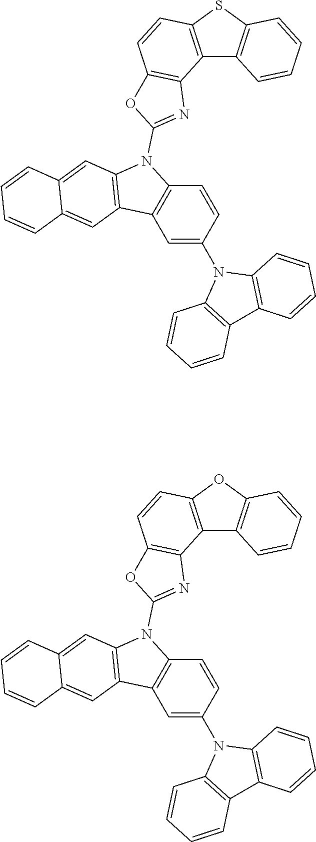 [SMILES: c1ccc2cc3c(cc2c1)c1cc(-n2c4ccccc4c4ccccc42)ccc1n3-c1nc2c(ccc3sc4ccccc4c32)o1 ; c1ccc2cc3c(cc2c1)c1cc(-n2c4ccccc4c4ccccc42)ccc1n3-c1nc2c(ccc3oc4ccccc4c32)o1]

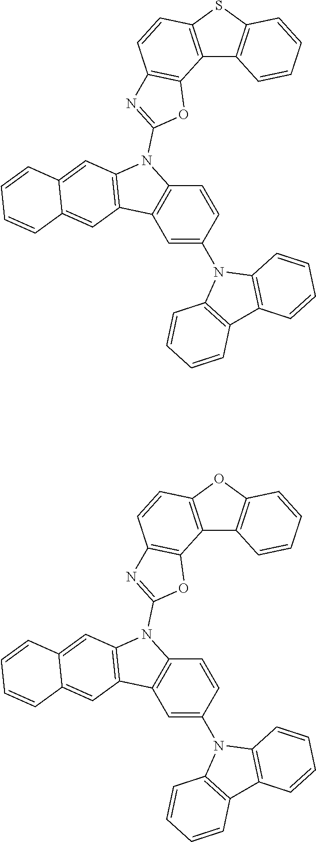 [SMILES: c1ccc2cc3c(cc2c1)c1cc(-n2c4ccccc4c4ccccc42)ccc1n3-c1nc2ccc3sc4ccccc4c3c2o1 ; c1ccc2cc3c(cc2c1)c1cc(-n2c4ccccc4c4ccccc42)ccc1n3-c1nc2ccc3oc4ccccc4c3c2o1]

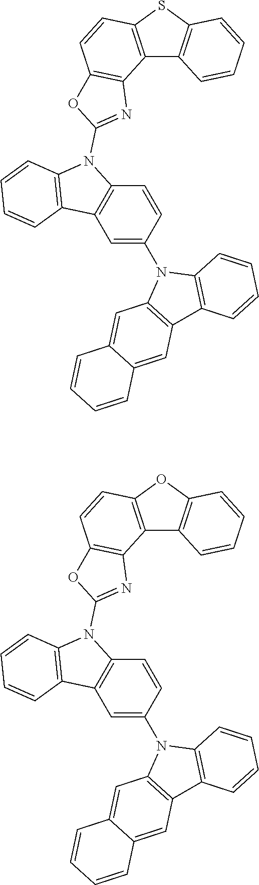 [SMILES: c1ccc2cc3c(cc2c1)c1ccccc1n3-c1ccc2c(c1)c1ccccc1n2-c1nc2c(ccc3sc4ccccc4c32)o1 ; c1ccc2cc3c(cc2c1)c1ccccc1n3-c1ccc2c(c1)c1ccccc1n2-c1nc2c(ccc3oc4ccccc4c32)o1]

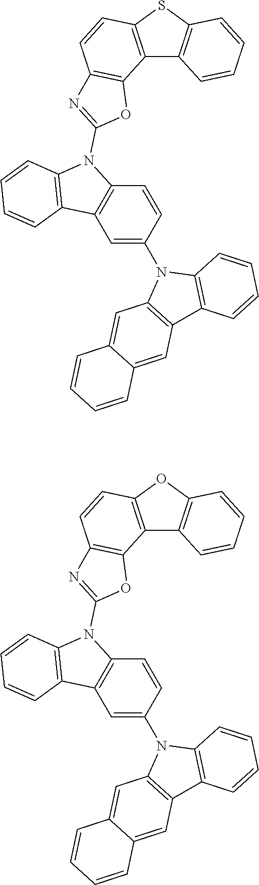 [SMILES: c1ccc2cc3c(cc2c1)c1ccccc1n3-c1ccc2c(c1)c1ccccc1n2-c1nc2ccc3sc4ccccc4c3c2o1 ; c1ccc2cc3c(cc2c1)c1ccccc1n3-c1ccc2c(c1)c1ccccc1n2-c1nc2ccc3oc4ccccc4c3c2o1]

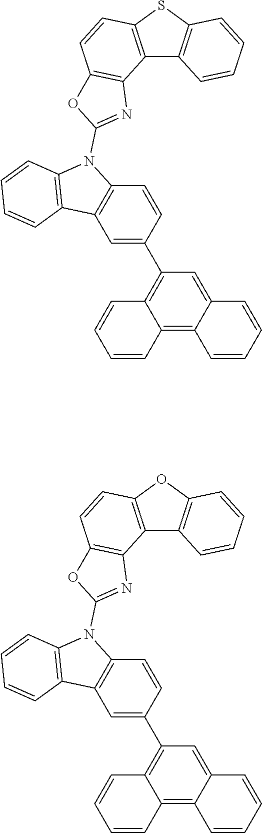 [SMILES: c1ccc2c(c1)cc(-c1ccc3c(c1)c1ccccc1n3-c1nc3c(ccc4sc5ccccc5c43)o1)c1ccccc12 ; c1ccc2c(c1)cc(-c1ccc3c(c1)c1ccccc1n3-c1nc3c(ccc4oc5ccccc5c43)o1)c1ccccc12]

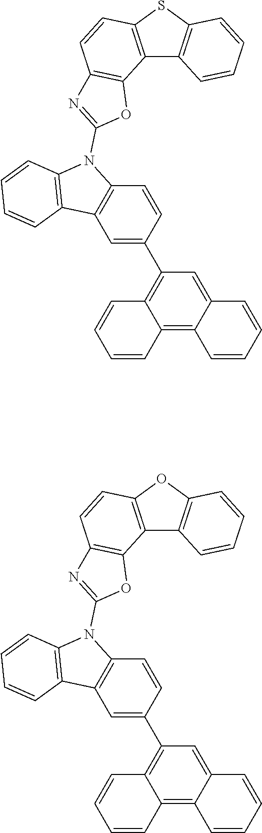 [SMILES: c1ccc2c(c1)cc(-c1ccc3c(c1)c1ccccc1n3-c1nc3ccc4sc5ccccc5c4c3o1)c1ccccc12 ; c1ccc2c(c1)cc(-c1ccc3c(c1)c1ccccc1n3-c1nc3ccc4oc5ccccc5c4c3o1)c1ccccc12]

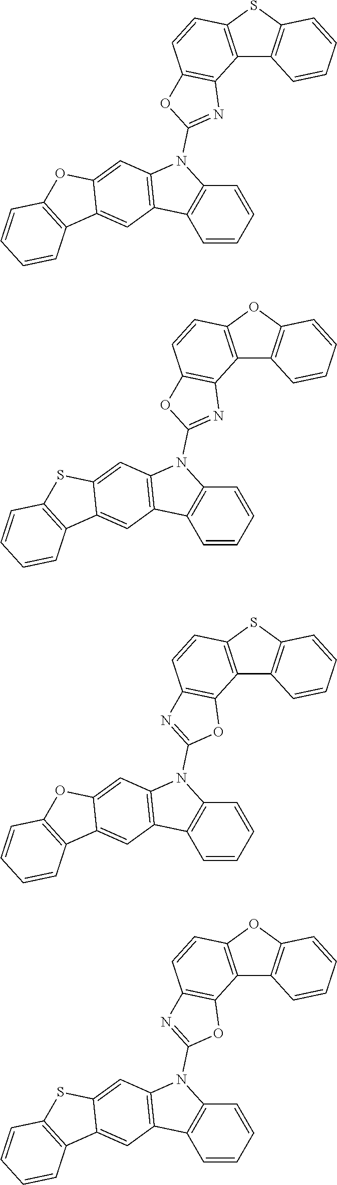 [SMILES: c1ccc2c(c1)oc1cc3c(cc12)c1ccccc1n3-c1nc2c(ccc3sc4ccccc4c32)o1 ; c1ccc2c(c1)oc1ccc3oc(-n4c5ccccc5c5cc6c(cc54)sc4ccccc46)nc3c12 ; c1ccc2c(c1)oc1cc3c(cc12)c1ccccc1n3-c1nc2ccc3sc4ccccc4c3c2o1 ; c1ccc2c(c1)oc1ccc3nc(-n4c5ccccc5c5cc6c(cc54)sc4ccccc46)oc3c12]

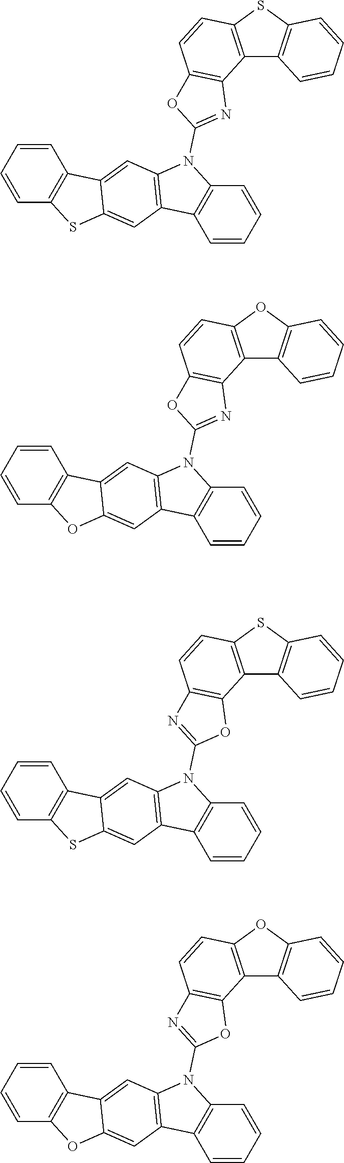 [SMILES: c1ccc2c(c1)sc1cc3c4ccccc4n(-c4nc5c(ccc6sc7ccccc7c65)o4)c3cc12 ; c1ccc2c(c1)oc1cc3c4ccccc4n(-c4nc5c(ccc6oc7ccccc7c65)o4)c3cc12 ; c1ccc2c(c1)sc1cc3c4ccccc4n(-c4nc5ccc6sc7ccccc7c6c5o4)c3cc12 ; c1ccc2c(c1)oc1cc3c4ccccc4n(-c4nc5ccc6oc7ccccc7c6c5o4)c3cc12]

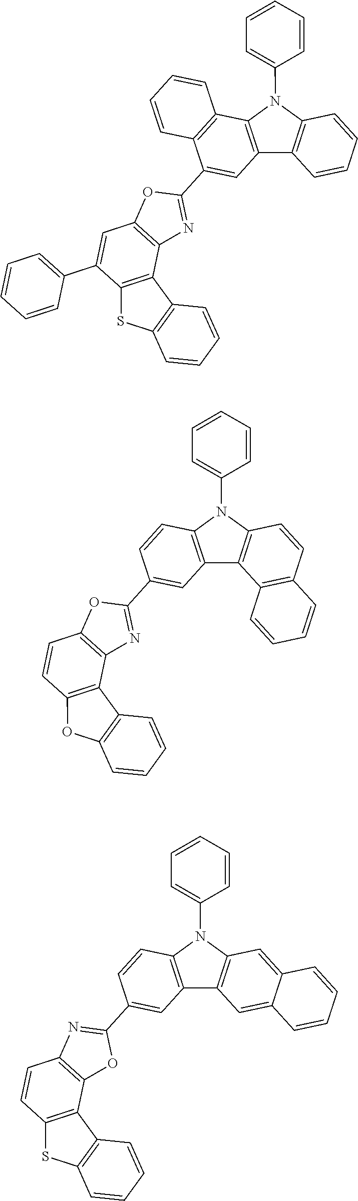 [SMILES: c1ccc(-c2cc3oc(-c4cc5c6ccccc6n(-c6ccccc6)c5c5ccccc45)nc3c3c2sc2ccccc23)cc1 ; c1ccc(-n2c3ccc(-c4nc5c(ccc6oc7ccccc7c65)o4)cc3c3c4ccccc4ccc32)cc1 ; c1ccc(-n2c3ccc(-c4nc5ccc6sc7ccccc7c6c5o4)cc3c3cc4ccccc4cc32)cc1]

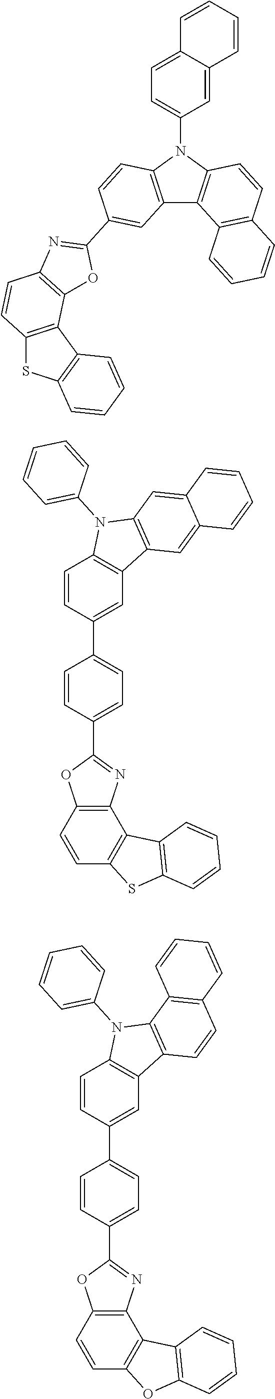

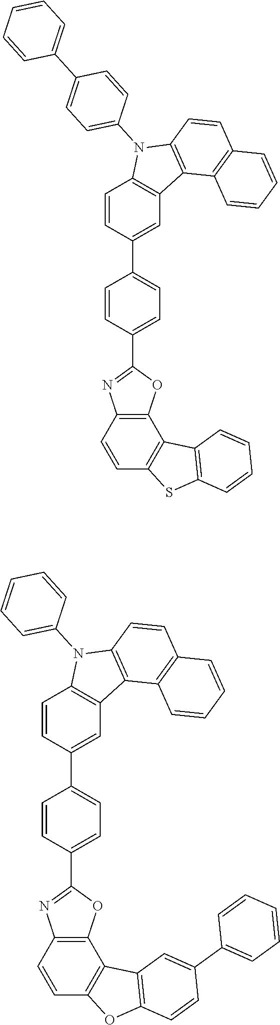 [SMILES: c1ccc(-c2ccc(-n3c4ccc(-c5ccc(-c6nc7ccc8sc9ccccc9c8c7o6)cc5)cc4c4c5ccccc5ccc43)cc2)cc1 ; c1ccc(-c2ccc3oc4ccc5nc(-c6ccc(-c7ccc8c(c7)c7c9ccccc9ccc7n8-c7ccccc7)cc6)oc5c4c3c2)cc1]

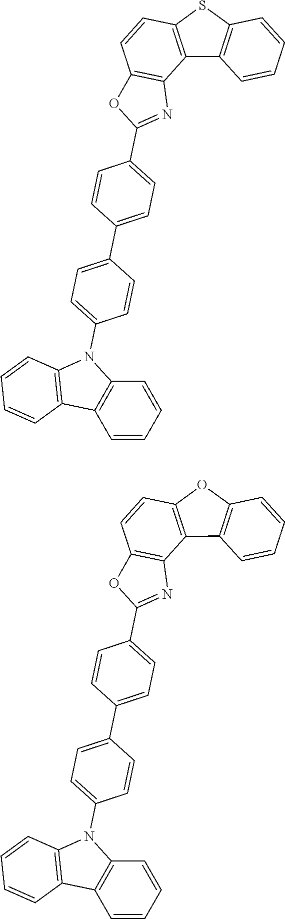 [SMILES: c1ccc2c(c1)sc1ccc3oc(-c4ccc(-c5ccc(-n6c7ccccc7c7ccccc76)cc5)cc4)nc3c12 ; c1ccc2c(c1)oc1ccc3oc(-c4ccc(-c5ccc(-n6c7ccccc7c7ccccc76)cc5)cc4)nc3c12]

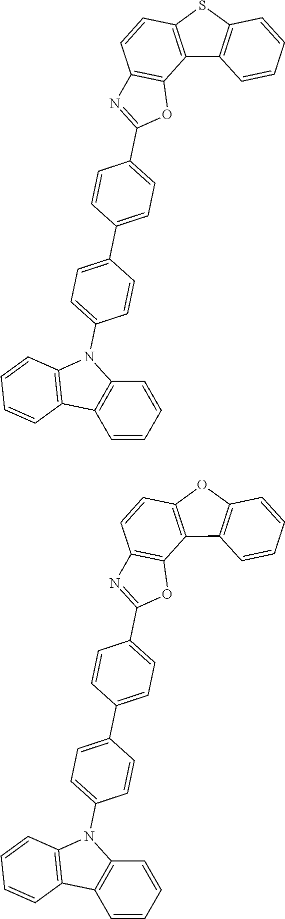 [SMILES: c1ccc2c(c1)sc1ccc3nc(-c4ccc(-c5ccc(-n6c7ccccc7c7ccccc76)cc5)cc4)oc3c12 ; c1ccc2c(c1)oc1ccc3nc(-c4ccc(-c5ccc(-n6c7ccccc7c7ccccc76)cc5)cc4)oc3c12]

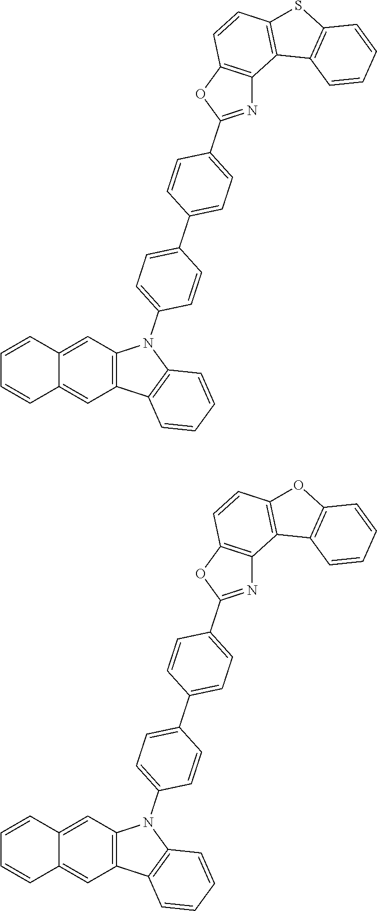 [SMILES: c1ccc2cc3c(cc2c1)c1ccccc1n3-c1ccc(-c2ccc(-c3nc4c(ccc5sc6ccccc6c54)o3)cc2)cc1 ; c1ccc2cc3c(cc2c1)c1ccccc1n3-c1ccc(-c2ccc(-c3nc4c(ccc5oc6ccccc6c54)o3)cc2)cc1]

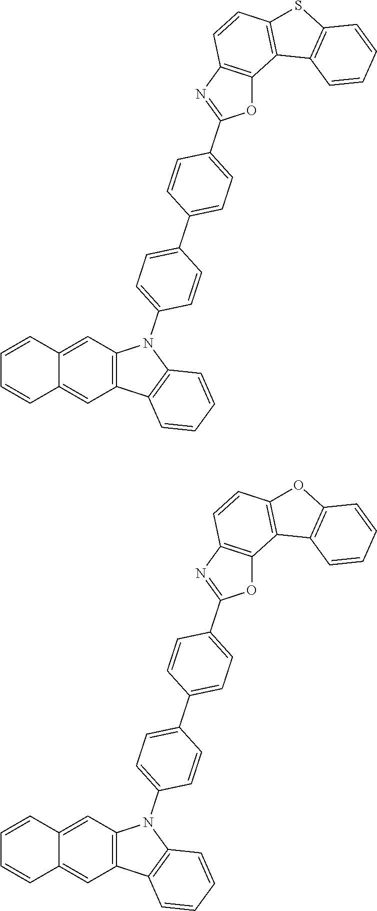 [SMILES: c1ccc2cc3c(cc2c1)c1ccccc1n3-c1ccc(-c2ccc(-c3nc4ccc5sc6ccccc6c5c4o3)cc2)cc1 ; c1ccc2cc3c(cc2c1)c1ccccc1n3-c1ccc(-c2ccc(-c3nc4ccc5oc6ccccc6c5c4o3)cc2)cc1]

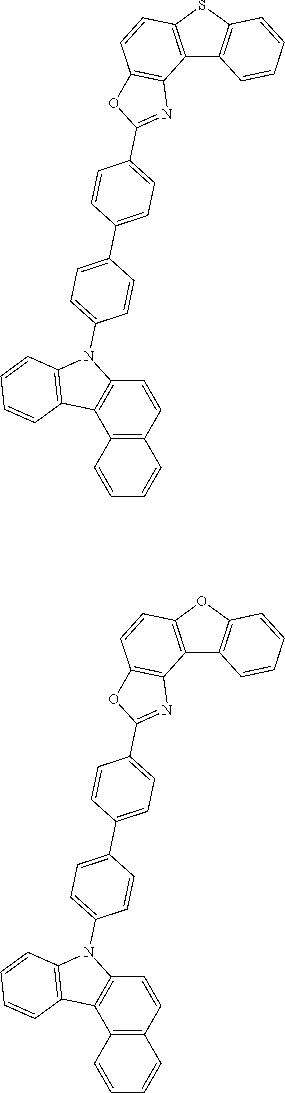 [SMILES: c1ccc2c(c1)ccc1c2c2ccccc2n1-c1ccc(-c2ccc(-c3nc4c(ccc5sc6ccccc6c54)o3)cc2)cc1 ; c1ccc2c(c1)ccc1c2c2ccccc2n1-c1ccc(-c2ccc(-c3nc4c(ccc5oc6ccccc6c54)o3)cc2)cc1]

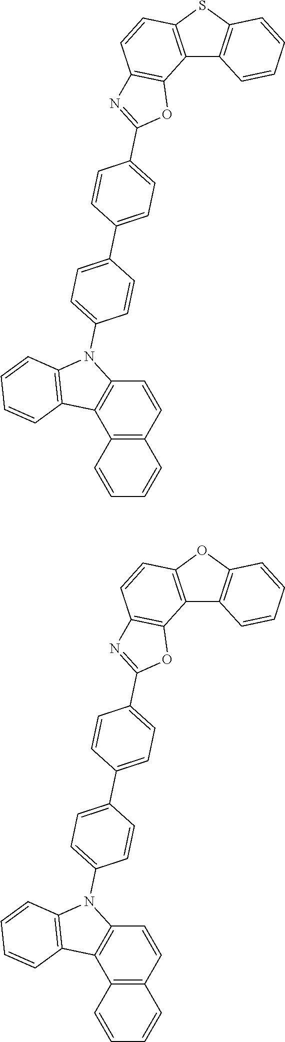 [SMILES: c1ccc2c(c1)ccc1c2c2ccccc2n1-c1ccc(-c2ccc(-c3nc4ccc5sc6ccccc6c5c4o3)cc2)cc1 ; c1ccc2c(c1)ccc1c2c2ccccc2n1-c1ccc(-c2ccc(-c3nc4ccc5oc6ccccc6c5c4o3)cc2)cc1]

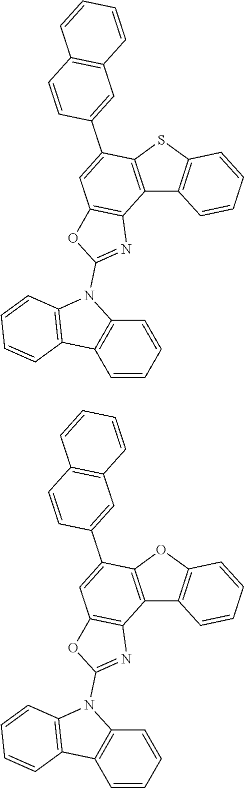

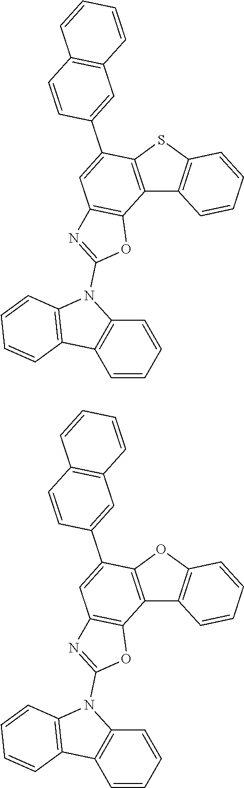

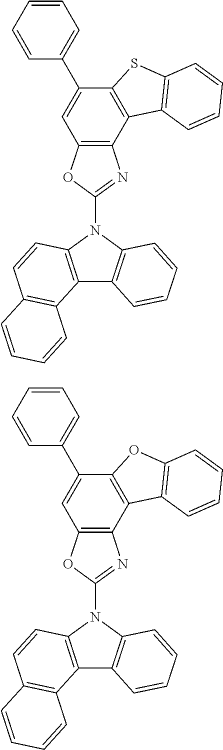

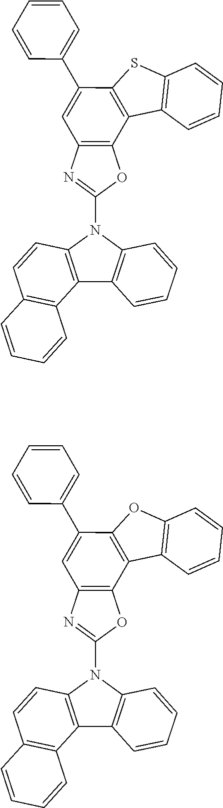 [SMILES: c1ccc(-c2cc3nc(-n4c5ccccc5c5c6ccccc6ccc54)oc3c3c2sc2ccccc23)cc1 ; c1ccc(-c2cc3nc(-n4c5ccccc5c5c6ccccc6ccc54)oc3c3c2oc2ccccc23)cc1]

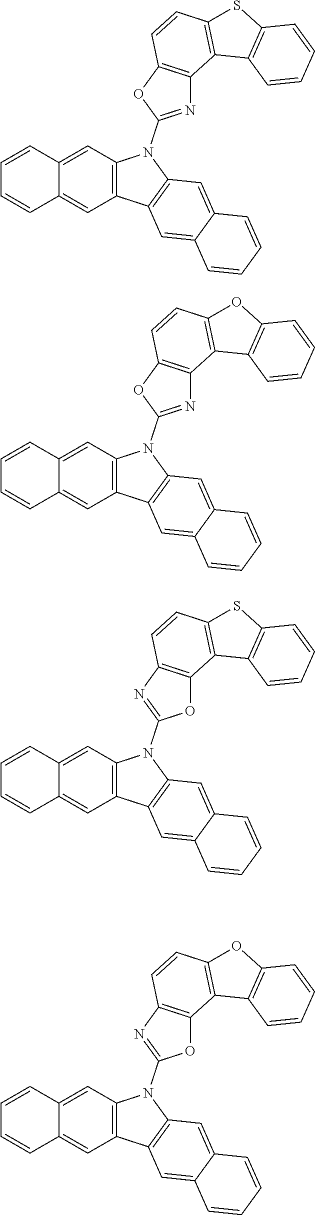

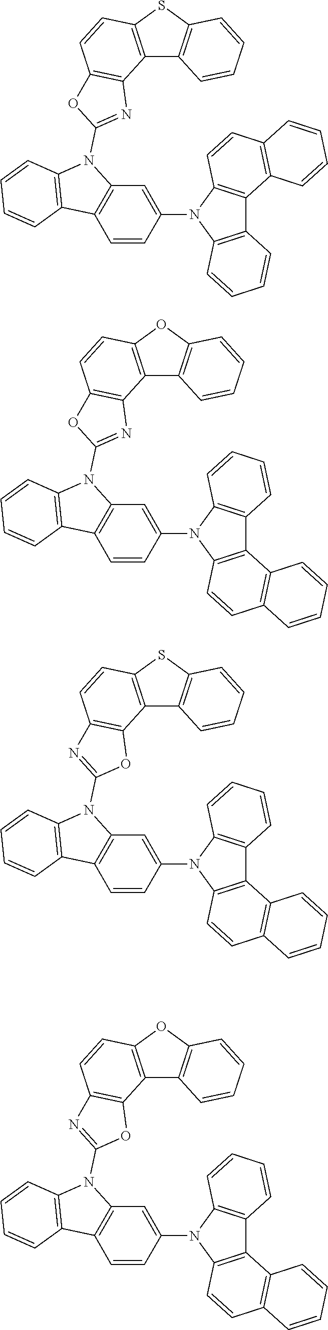

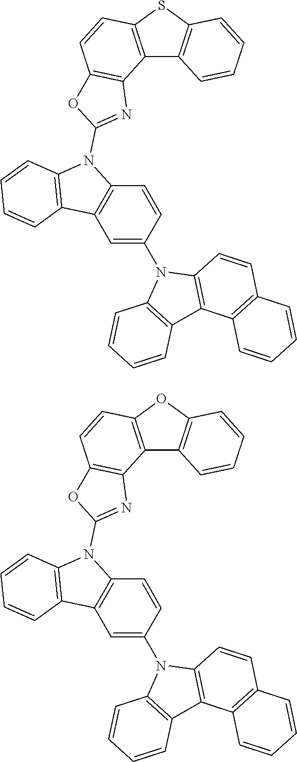

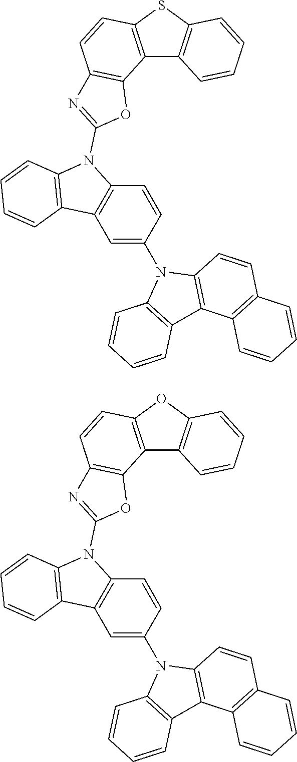

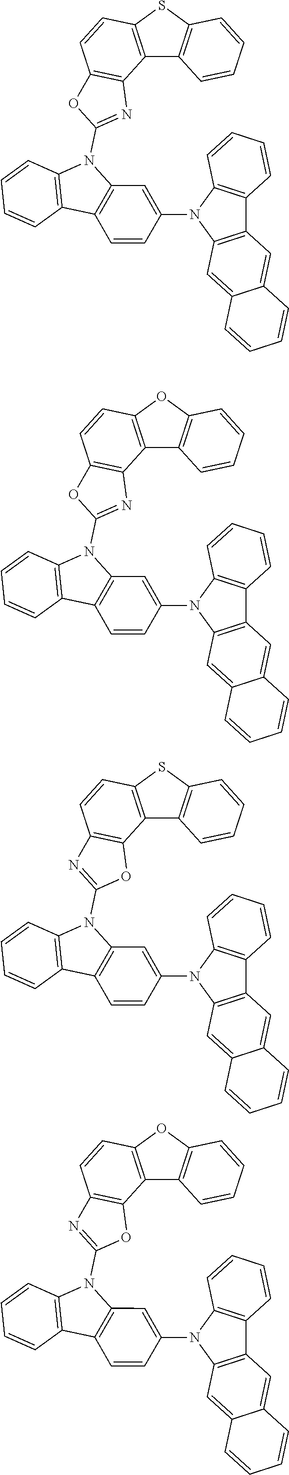

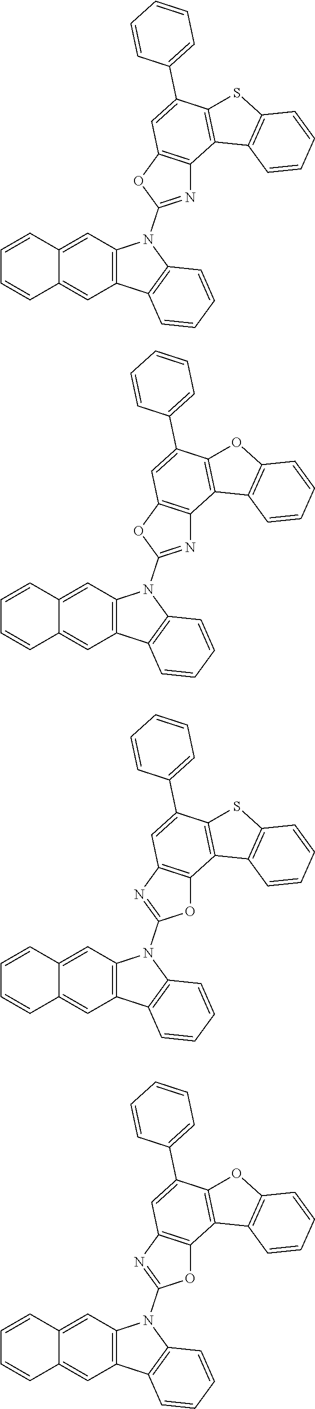

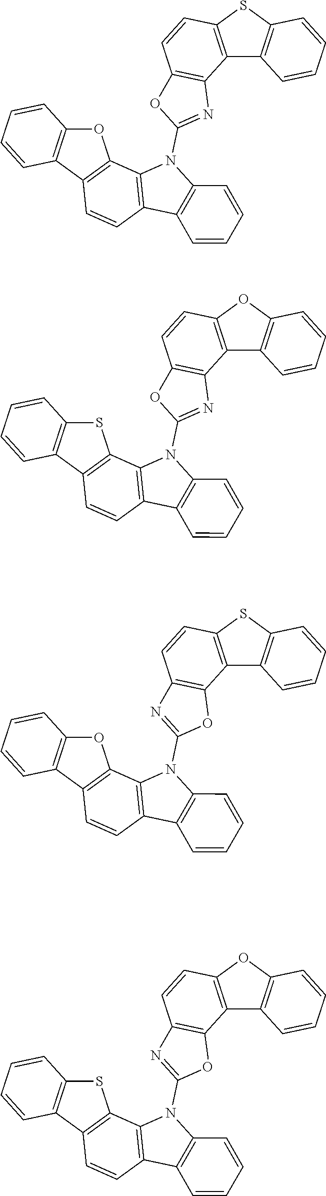 [SMILES: c1ccc2c(c1)oc1c2ccc2c3ccccc3n(-c3nc4c(ccc5sc6ccccc6c54)o3)c21 ; c1ccc2c(c1)oc1ccc3oc(-n4c5ccccc5c5ccc6c7ccccc7sc6c54)nc3c12 ; c1ccc2c(c1)oc1c2ccc2c3ccccc3n(-c3nc4ccc5sc6ccccc6c5c4o3)c21 ; c1ccc2c(c1)oc1ccc3nc(-n4c5ccccc5c5ccc6c7ccccc7sc6c54)oc3c12]

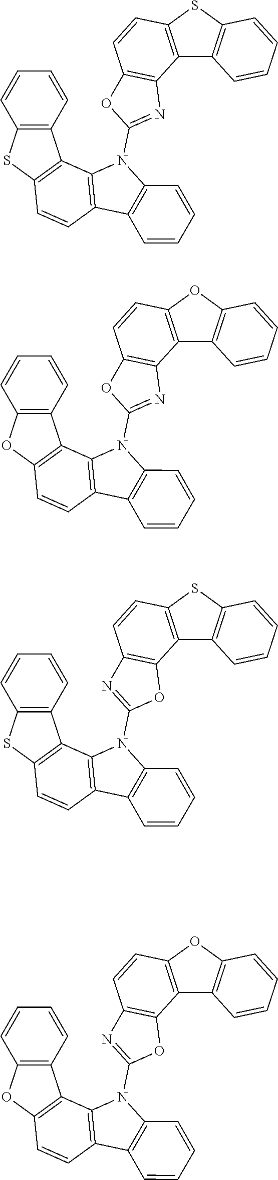 [SMILES: c1ccc2c(c1)sc1ccc3oc(-n4c5ccccc5c5ccc6sc7ccccc7c6c54)nc3c12 ; c1ccc2c(c1)oc1ccc3oc(-n4c5ccccc5c5ccc6oc7ccccc7c6c54)nc3c12 ; c1ccc2c(c1)sc1ccc3nc(-n4c5ccccc5c5ccc6sc7ccccc7c6c54)oc3c12 ; c1ccc2c(c1)oc1ccc3nc(-n4c5ccccc5c5ccc6oc7ccccc7c6c54)oc3c12]

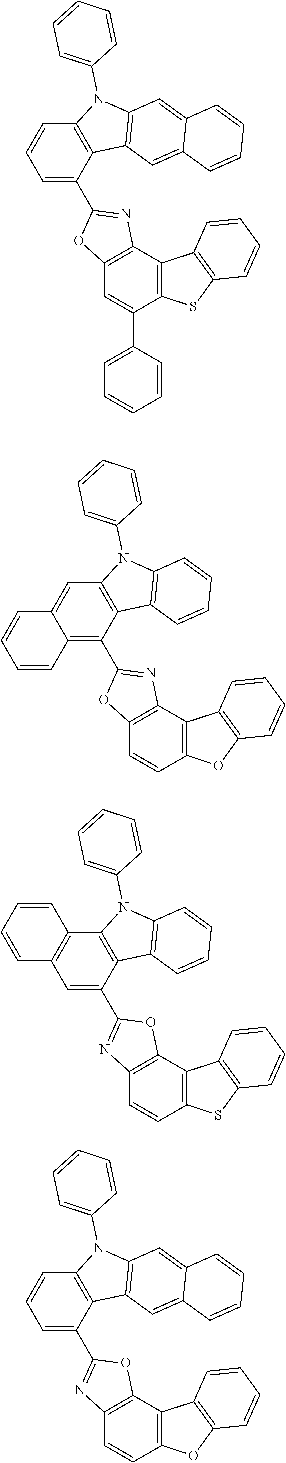 [SMILES: c1ccc(-c2cc3oc(-c4cccc5c4c4cc6ccccc6cc4n5-c4ccccc4)nc3c3c2sc2ccccc23)cc1 ; c1ccc(-n2c3ccccc3c3c(-c4nc5c(ccc6oc7ccccc7c65)o4)c4ccccc4cc32)cc1 ; c1ccc(-n2c3ccccc3c3c(-c4nc5ccc6sc7ccccc7c6c5o4)cc4ccccc4c32)cc1 ; c1ccc(-n2c3cc4ccccc4cc3c3c(-c4nc5ccc6oc7ccccc7c6c5o4)cccc32)cc1]

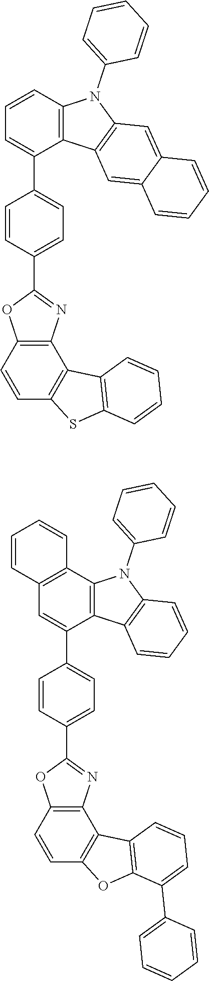 [SMILES: c1ccc(-n2c3cc4ccccc4cc3c3c(-c4ccc(-c5nc6c(ccc7sc8ccccc8c76)o5)cc4)cccc32)cc1 ; c1ccc(-c2cccc3c2oc2ccc4oc(-c5ccc(-c6cc7ccccc7c7c6c6ccccc6n7-c6ccccc6)cc5)nc4c23)cc1]

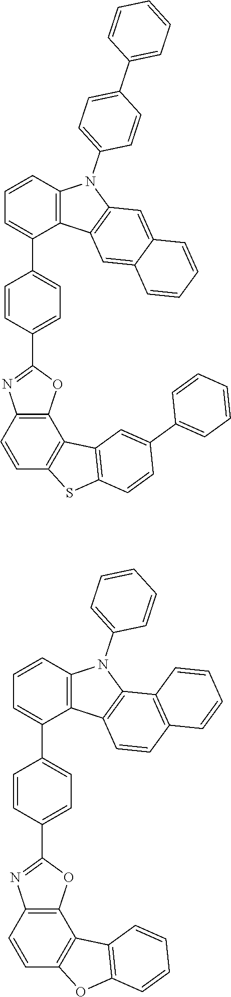 [SMILES: c1ccc(-c2ccc(-n3c4cc5ccccc5cc4c4c(-c5ccc(-c6nc7ccc8sc9ccc(-c%10ccccc%10)cc9c8c7o6)cc5)cccc43)cc2)cc1 ; c1ccc(-n2c3cccc(-c4ccc(-c5nc6ccc7oc8ccccc8c7c6o5)cc4)c3c3ccc4ccccc4c32)cc1]

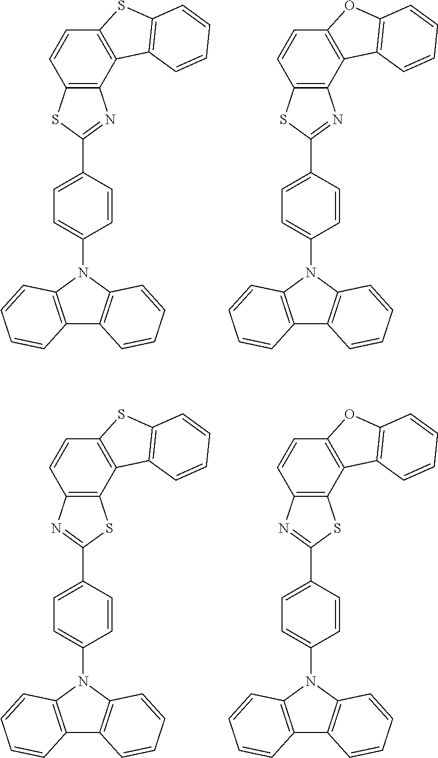 [SMILES: c1ccc2c(c1)sc1ccc3sc(-c4ccc(-n5c6ccccc6c6ccccc65)cc4)nc3c12 ; c1ccc2c(c1)oc1ccc3sc(-c4ccc(-n5c6ccccc6c6ccccc65)cc4)nc3c12 ; c1ccc2c(c1)sc1ccc3nc(-c4ccc(-n5c6ccccc6c6ccccc65)cc4)sc3c12 ; c1ccc2c(c1)oc1ccc3nc(-c4ccc(-n5c6ccccc6c6ccccc65)cc4)sc3c12]

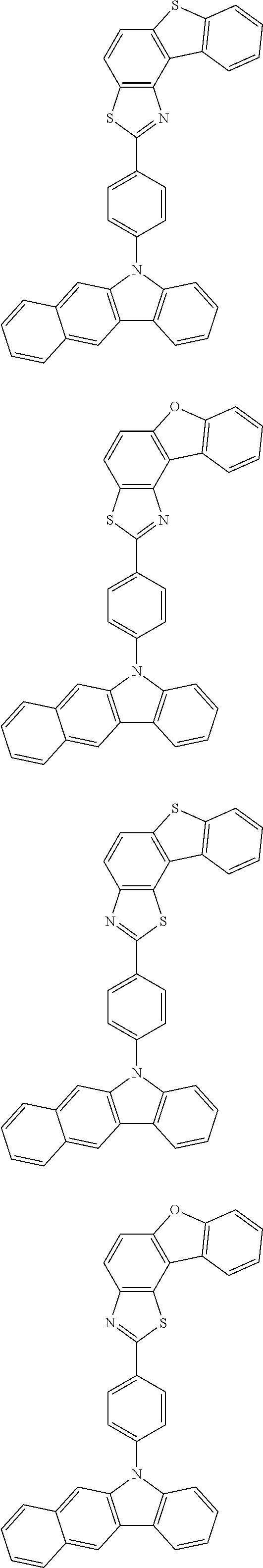 [SMILES: c1ccc2cc3c(cc2c1)c1ccccc1n3-c1ccc(-c2nc3c(ccc4sc5ccccc5c43)s2)cc1 ; c1ccc2cc3c(cc2c1)c1ccccc1n3-c1ccc(-c2nc3c(ccc4oc5ccccc5c43)s2)cc1 ; c1ccc2cc3c(cc2c1)c1ccccc1n3-c1ccc(-c2nc3ccc4sc5ccccc5c4c3s2)cc1 ; c1ccc2cc3c(cc2c1)c1ccccc1n3-c1ccc(-c2nc3ccc4oc5ccccc5c4c3s2)cc1]

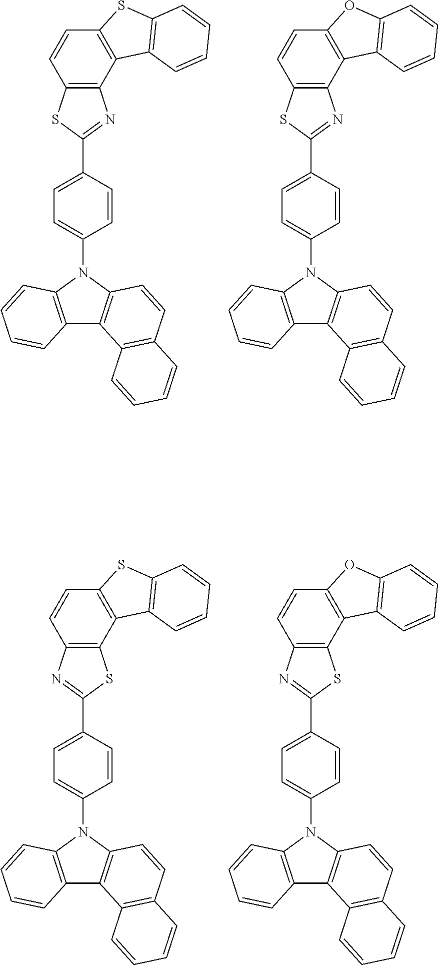 [SMILES: c1ccc2c(c1)ccc1c2c2ccccc2n1-c1ccc(-c2nc3c(ccc4sc5ccccc5c43)s2)cc1 ; c1ccc2c(c1)ccc1c2c2ccccc2n1-c1ccc(-c2nc3c(ccc4oc5ccccc5c43)s2)cc1 ; c1ccc2c(c1)ccc1c2c2ccccc2n1-c1ccc(-c2nc3ccc4sc5ccccc5c4c3s2)cc1 ; c1ccc2c(c1)ccc1c2c2ccccc2n1-c1ccc(-c2nc3ccc4oc5ccccc5c4c3s2)cc1]

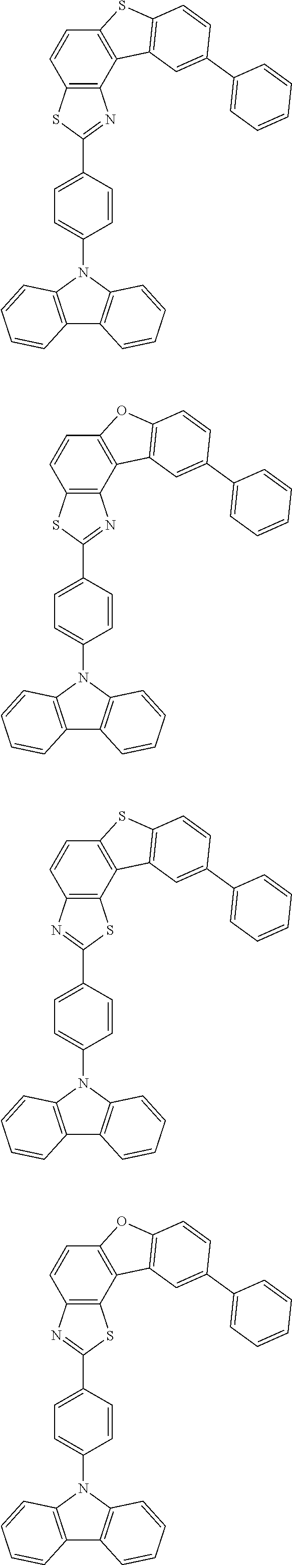 [SMILES: c1ccc(-c2ccc3sc4ccc5sc(-c6ccc(-n7c8ccccc8c8ccccc87)cc6)nc5c4c3c2)cc1 ; c1ccc(-c2ccc3oc4ccc5sc(-c6ccc(-n7c8ccccc8c8ccccc87)cc6)nc5c4c3c2)cc1 ; c1ccc(-c2ccc3sc4ccc5nc(-c6ccc(-n7c8ccccc8c8ccccc87)cc6)sc5c4c3c2)cc1 ; c1ccc(-c2ccc3oc4ccc5nc(-c6ccc(-n7c8ccccc8c8ccccc87)cc6)sc5c4c3c2)cc1]

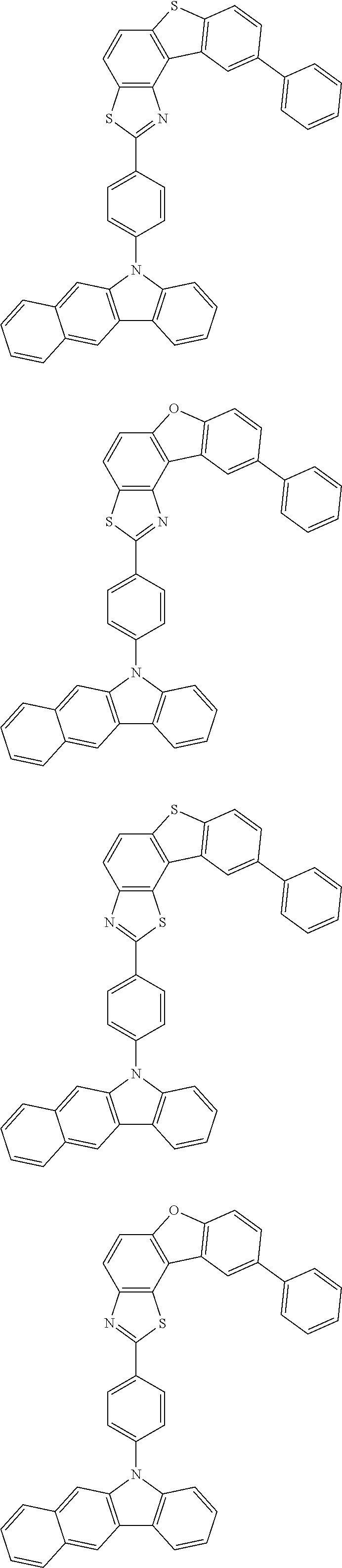 [SMILES: c1ccc(-c2ccc3sc4ccc5sc(-c6ccc(-n7c8ccccc8c8cc9ccccc9cc87)cc6)nc5c4c3c2)cc1 ; c1ccc(-c2ccc3oc4ccc5sc(-c6ccc(-n7c8ccccc8c8cc9ccccc9cc87)cc6)nc5c4c3c2)cc1 ; c1ccc(-c2ccc3sc4ccc5nc(-c6ccc(-n7c8ccccc8c8cc9ccccc9cc87)cc6)sc5c4c3c2)cc1 ; c1ccc(-c2ccc3oc4ccc5nc(-c6ccc(-n7c8ccccc8c8cc9ccccc9cc87)cc6)sc5c4c3c2)cc1]

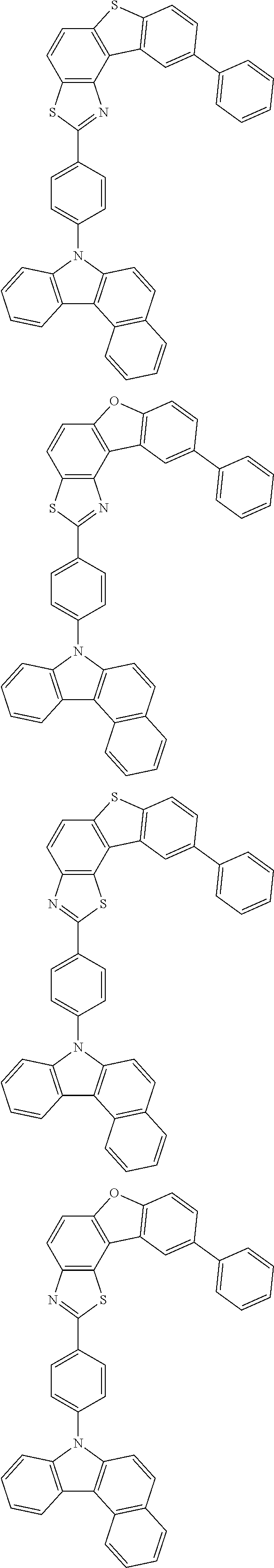

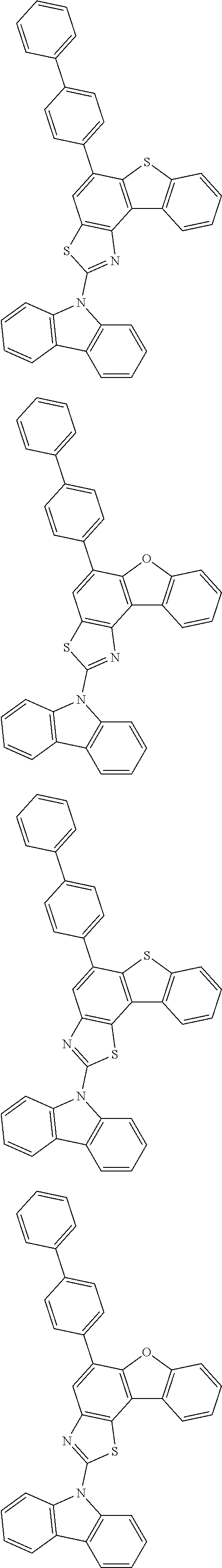

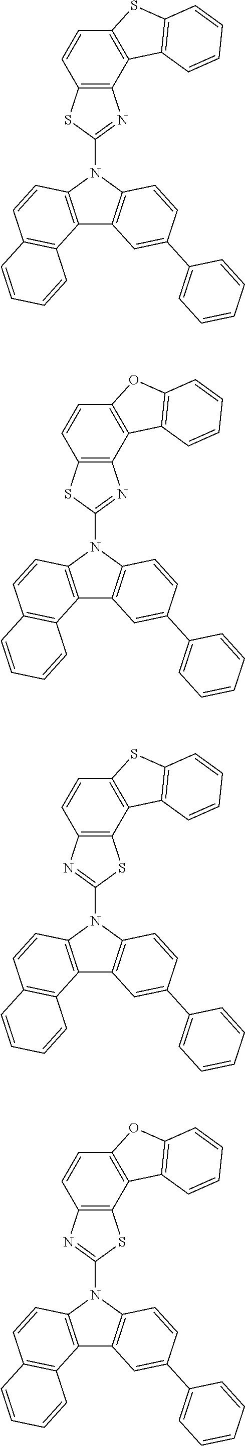 [SMILES: c1ccc(-c2ccc3c(c2)c2c4ccccc4ccc2n3-c2nc3c(ccc4sc5ccccc5c43)s2)cc1 ; c1ccc(-c2ccc3c(c2)c2c4ccccc4ccc2n3-c2nc3c(ccc4oc5ccccc5c43)s2)cc1 ; c1ccc(-c2ccc3c(c2)c2c4ccccc4ccc2n3-c2nc3ccc4sc5ccccc5c4c3s2)cc1 ; c1ccc(-c2ccc3c(c2)c2c4ccccc4ccc2n3-c2nc3ccc4oc5ccccc5c4c3s2)cc1]

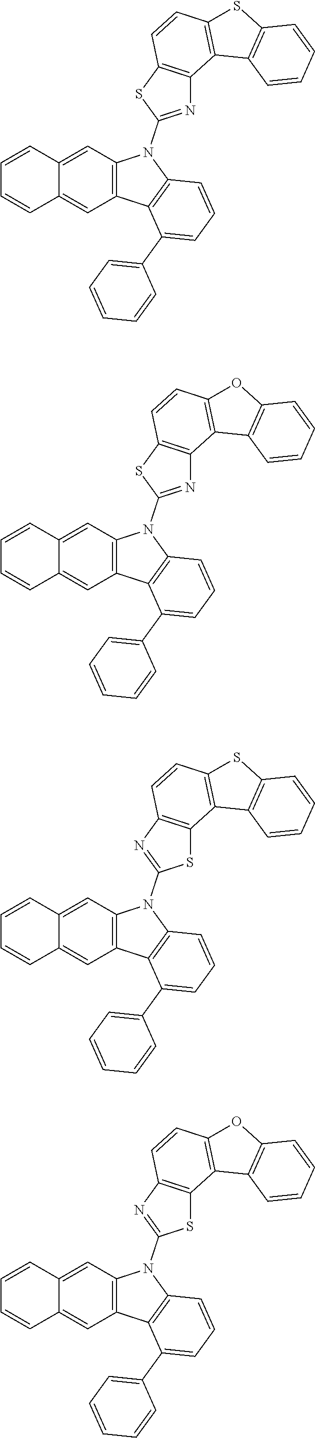 [SMILES: c1ccc(-c2cccc3c2c2cc4ccccc4cc2n3-c2nc3c(ccc4sc5ccccc5c43)s2)cc1 ; c1ccc(-c2cccc3c2c2cc4ccccc4cc2n3-c2nc3c(ccc4oc5ccccc5c43)s2)cc1 ; c1ccc(-c2cccc3c2c2cc4ccccc4cc2n3-c2nc3ccc4sc5ccccc5c4c3s2)cc1 ; c1ccc(-c2cccc3c2c2cc4ccccc4cc2n3-c2nc3ccc4oc5ccccc5c4c3s2)cc1]

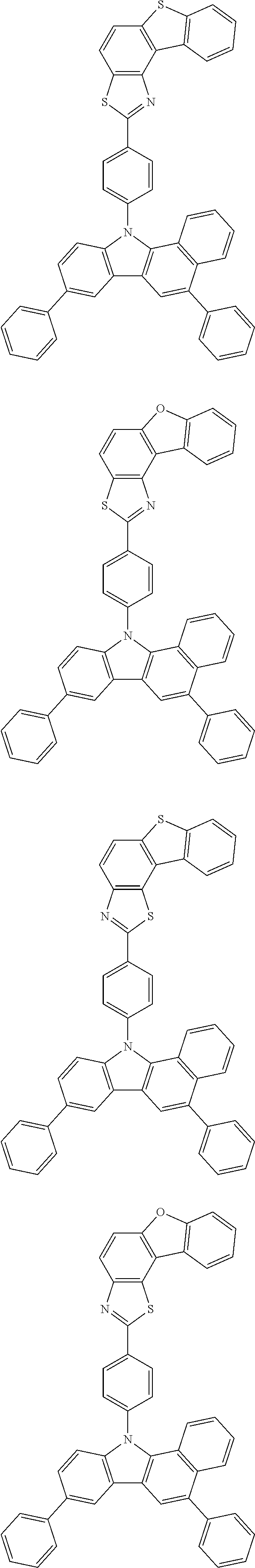

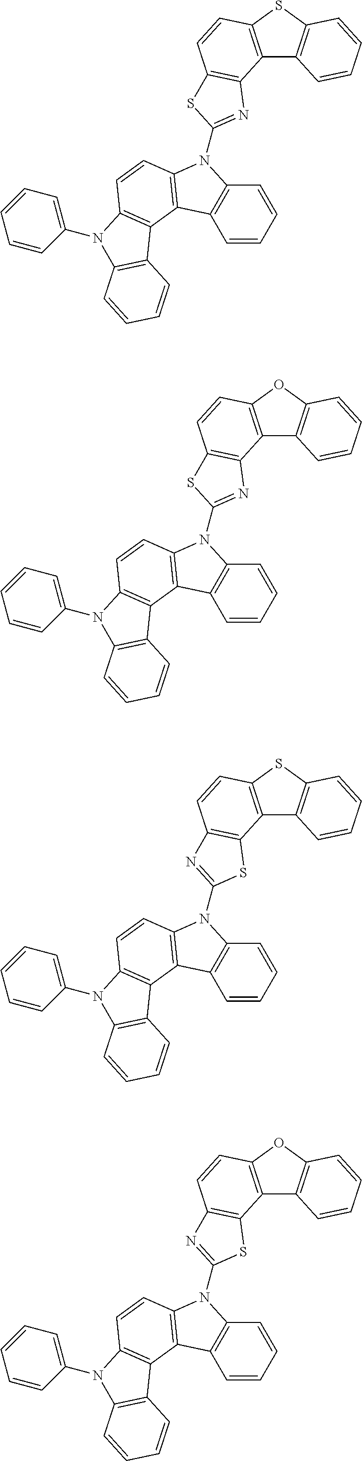 [SMILES: c1ccc(-n2c3ccccc3c3c4c5ccccc5n(-c5nc6c(ccc7sc8ccccc8c76)s5)c4ccc32)cc1 ; c1ccc(-n2c3ccccc3c3c4c5ccccc5n(-c5nc6c(ccc7oc8ccccc8c76)s5)c4ccc32)cc1 ; c1ccc(-n2c3ccccc3c3c4c5ccccc5n(-c5nc6ccc7sc8ccccc8c7c6s5)c4ccc32)cc1 ; c1ccc(-n2c3ccccc3c3c4c5ccccc5n(-c5nc6ccc7oc8ccccc8c7c6s5)c4ccc32)cc1]

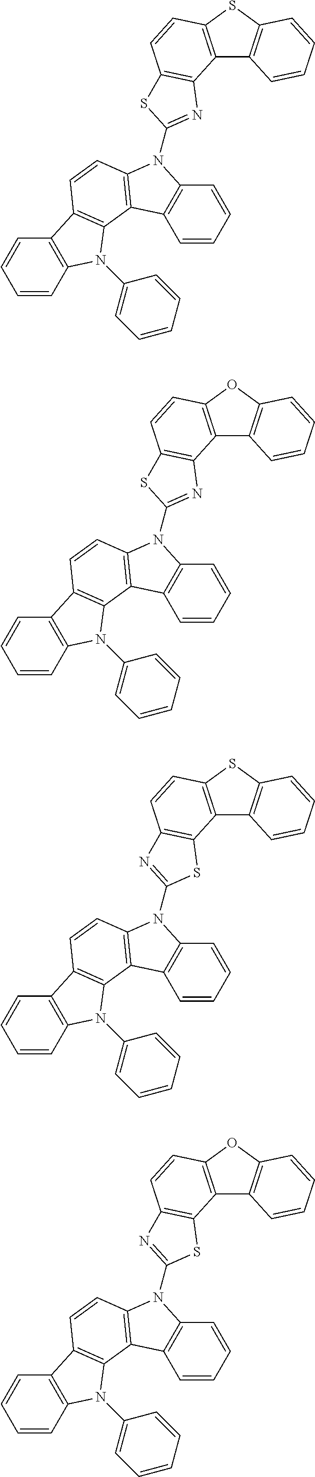 [SMILES: c1ccc(-n2c3ccccc3c3ccc4c(c5ccccc5n4-c4nc5c(ccc6sc7ccccc7c65)s4)c32)cc1 ; c1ccc(-n2c3ccccc3c3ccc4c(c5ccccc5n4-c4nc5c(ccc6oc7ccccc7c65)s4)c32)cc1 ; c1ccc(-n2c3ccccc3c3ccc4c(c5ccccc5n4-c4nc5ccc6sc7ccccc7c6c5s4)c32)cc1 ; c1ccc(-n2c3ccccc3c3ccc4c(c5ccccc5n4-c4nc5ccc6oc7ccccc7c6c5s4)c32)cc1]

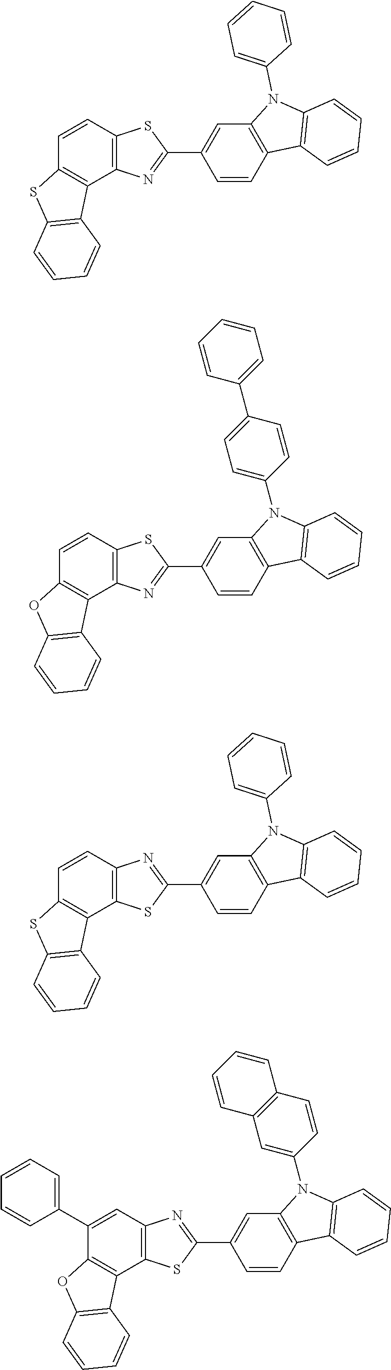 [SMILES: c1ccc(-n2c3ccccc3c3ccc(-c4nc5c(ccc6sc7ccccc7c65)s4)cc32)cc1 ; c1ccc(-c2ccc(-n3c4ccccc4c4ccc(-c5nc6c(ccc7oc8ccccc8c76)s5)cc43)cc2)cc1 ; c1ccc(-n2c3ccccc3c3ccc(-c4nc5ccc6sc7ccccc7c6c5s4)cc32)cc1 ; c1ccc(-c2cc3nc(-c4ccc5c6ccccc6n(-c6ccc7ccccc7c6)c5c4)sc3c3c2oc2ccccc23)cc1]

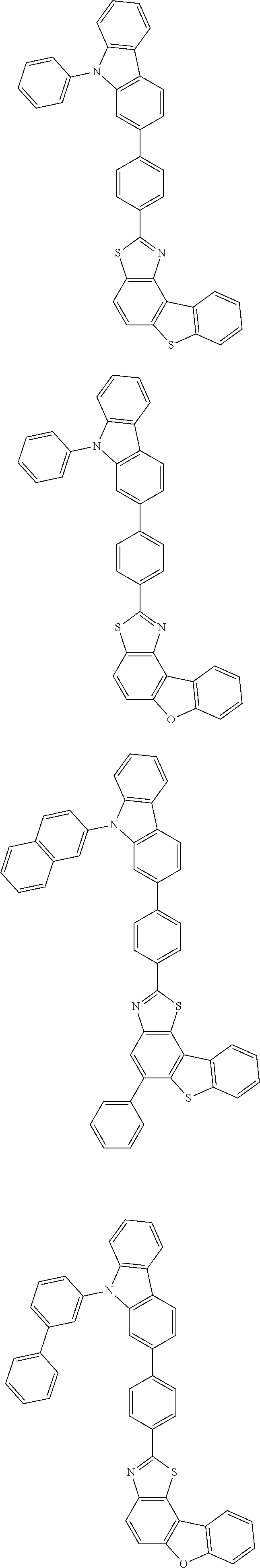 [SMILES: c1ccc(-n2c3ccccc3c3ccc(-c4ccc(-c5nc6c(ccc7sc8ccccc8c76)s5)cc4)cc32)cc1 ; c1ccc(-n2c3ccccc3c3ccc(-c4ccc(-c5nc6c(ccc7oc8ccccc8c76)s5)cc4)cc32)cc1 ; c1ccc(-c2cc3nc(-c4ccc(-c5ccc6c7ccccc7n(-c7ccc8ccccc8c7)c6c5)cc4)sc3c3c2sc2ccccc23)cc1 ; c1ccc(-c2cccc(-n3c4ccccc4c4ccc(-c5ccc(-c6nc7ccc8oc9ccccc9c8c7s6)cc5)cc43)c2)cc1]

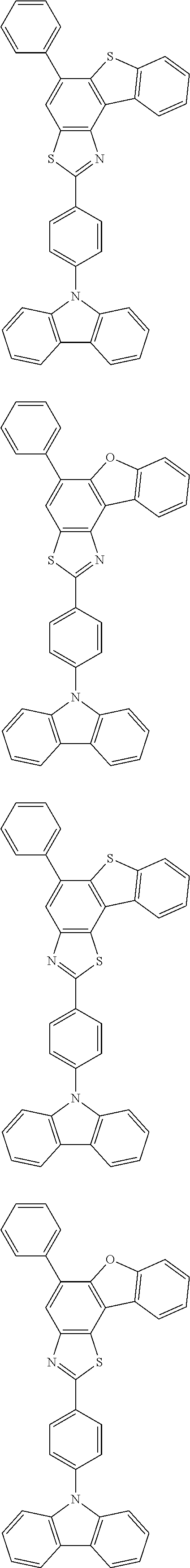

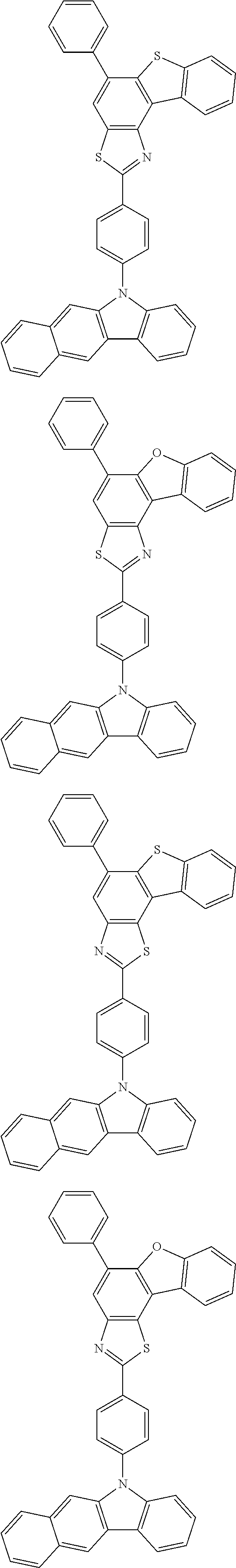

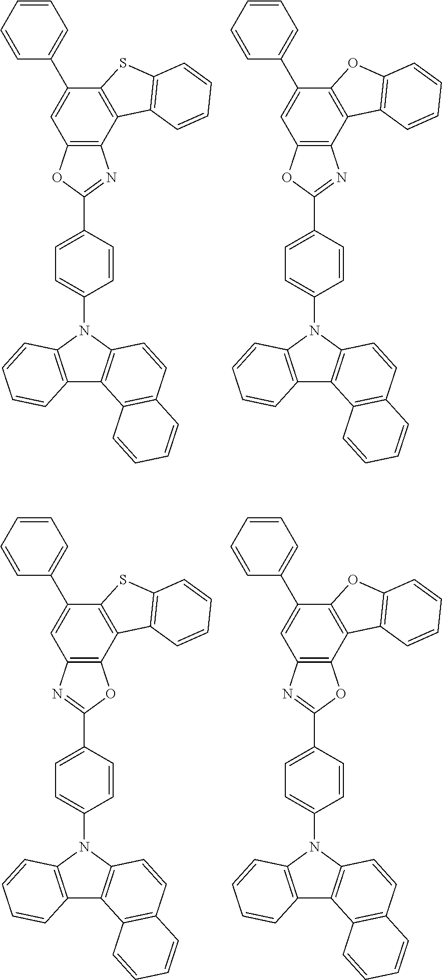

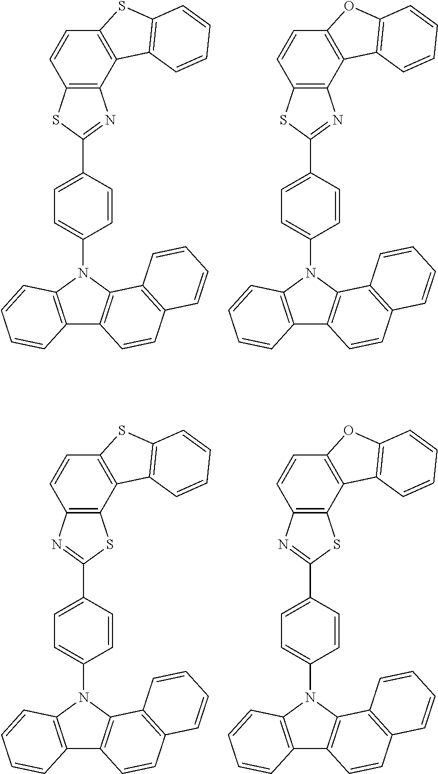

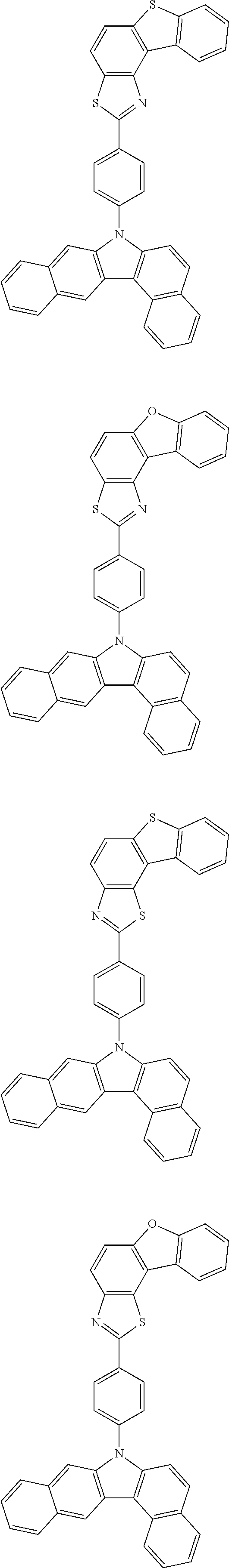 [SMILES: c1ccc2cc3c(cc2c1)c1c2ccccc2ccc1n3-c1ccc(-c2nc3c(ccc4sc5ccccc5c43)s2)cc1 ; c1ccc2cc3c(cc2c1)c1c2ccccc2ccc1n3-c1ccc(-c2nc3c(ccc4oc5ccccc5c43)s2)cc1 ; c1ccc2cc3c(cc2c1)c1c2ccccc2ccc1n3-c1ccc(-c2nc3ccc4sc5ccccc5c4c3s2)cc1 ; c1ccc2cc3c(cc2c1)c1c2ccccc2ccc1n3-c1ccc(-c2nc3ccc4oc5ccccc5c4c3s2)cc1]

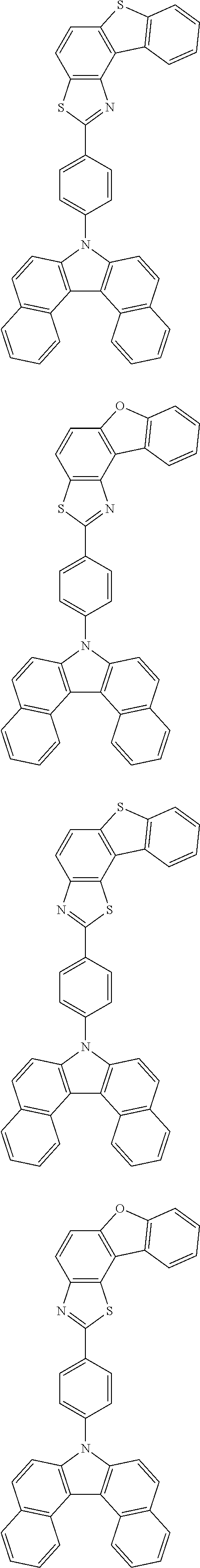 [SMILES: c1ccc2c(c1)ccc1c2c2c3ccccc3ccc2n1-c1ccc(-c2nc3c(ccc4sc5ccccc5c43)s2)cc1 ; c1ccc2c(c1)ccc1c2c2c3ccccc3ccc2n1-c1ccc(-c2nc3c(ccc4oc5ccccc5c43)s2)cc1 ; c1ccc2c(c1)ccc1c2c2c3ccccc3ccc2n1-c1ccc(-c2nc3ccc4sc5ccccc5c4c3s2)cc1 ; c1ccc2c(c1)ccc1c2c2c3ccccc3ccc2n1-c1ccc(-c2nc3ccc4oc5ccccc5c4c3s2)cc1]

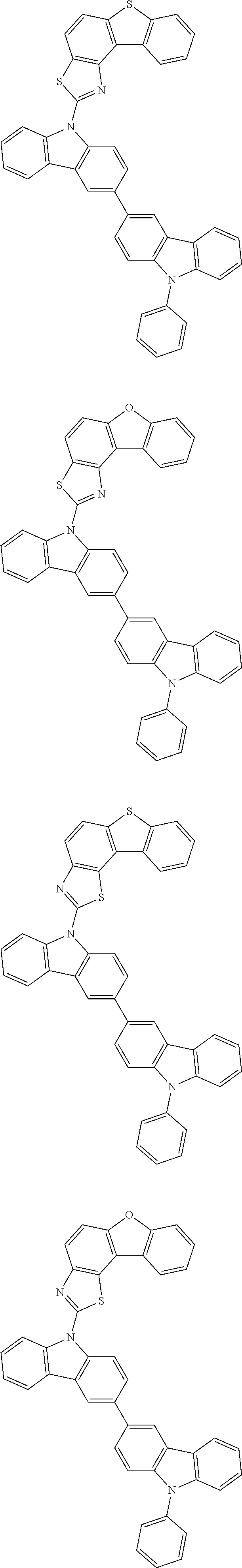 [SMILES: c1ccc(-n2c3ccccc3c3cc(-c4ccc5c(c4)c4ccccc4n5-c4nc5c(ccc6sc7ccccc7c65)s4)ccc32)cc1 ; c1ccc(-n2c3ccccc3c3cc(-c4ccc5c(c4)c4ccccc4n5-c4nc5c(ccc6oc7ccccc7c65)s4)ccc32)cc1 ; c1ccc(-n2c3ccccc3c3cc(-c4ccc5c(c4)c4ccccc4n5-c4nc5ccc6sc7ccccc7c6c5s4)ccc32)cc1 ; c1ccc(-n2c3ccccc3c3cc(-c4ccc5c(c4)c4ccccc4n5-c4nc5ccc6oc7ccccc7c6c5s4)ccc32)cc1]

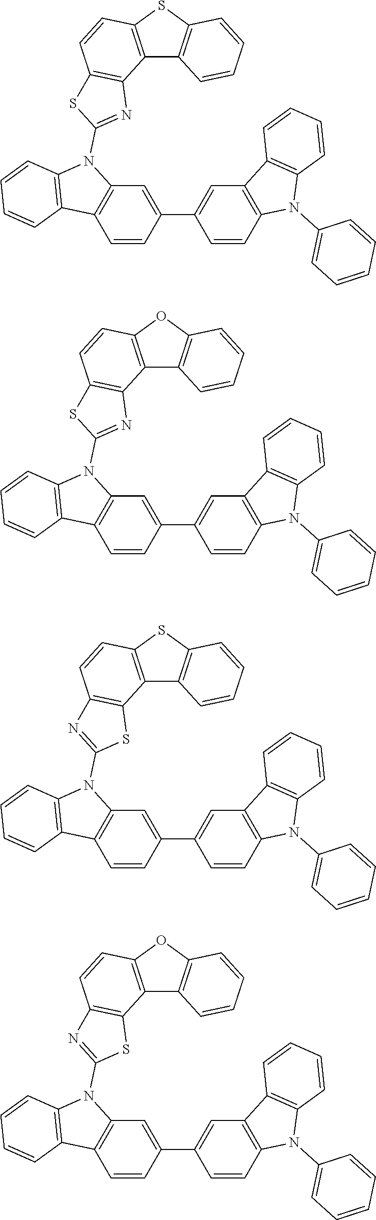 [SMILES: c1ccc(-n2c3ccccc3c3cc(-c4ccc5c6ccccc6n(-c6nc7c(ccc8sc9ccccc9c87)s6)c5c4)ccc32)cc1 ; c1ccc(-n2c3ccccc3c3cc(-c4ccc5c6ccccc6n(-c6nc7c(ccc8oc9ccccc9c87)s6)c5c4)ccc32)cc1 ; c1ccc(-n2c3ccccc3c3cc(-c4ccc5c6ccccc6n(-c6nc7ccc8sc9ccccc9c8c7s6)c5c4)ccc32)cc1 ; c1ccc(-n2c3ccccc3c3cc(-c4ccc5c6ccccc6n(-c6nc7ccc8oc9ccccc9c8c7s6)c5c4)ccc32)cc1]

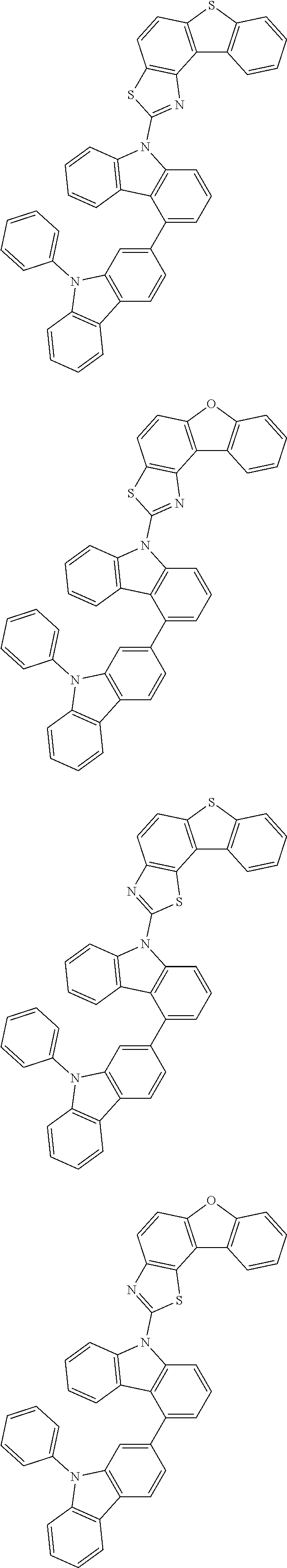 [SMILES: c1ccc(-n2c3ccccc3c3ccc(-c4cccc5c4c4ccccc4n5-c4nc5c(ccc6sc7ccccc7c65)s4)cc32)cc1 ; c1ccc(-n2c3ccccc3c3ccc(-c4cccc5c4c4ccccc4n5-c4nc5c(ccc6oc7ccccc7c65)s4)cc32)cc1 ; c1ccc(-n2c3ccccc3c3ccc(-c4cccc5c4c4ccccc4n5-c4nc5ccc6sc7ccccc7c6c5s4)cc32)cc1 ; c1ccc(-n2c3ccccc3c3ccc(-c4cccc5c4c4ccccc4n5-c4nc5ccc6oc7ccccc7c6c5s4)cc32)cc1]

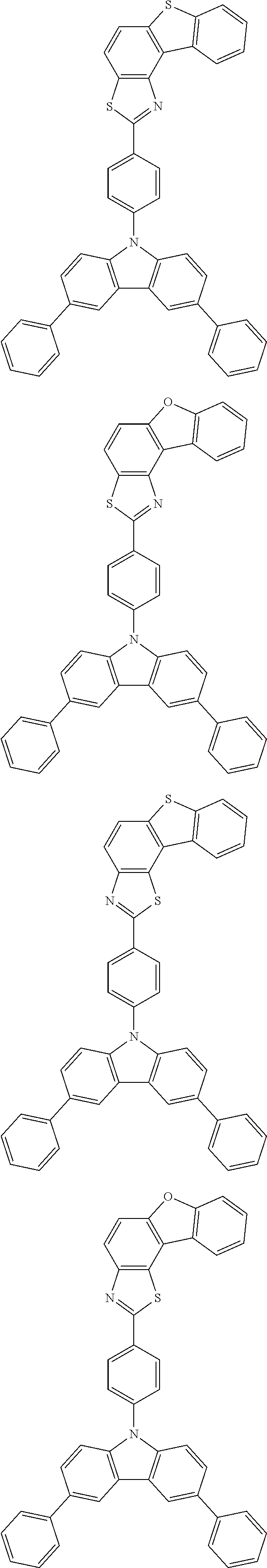

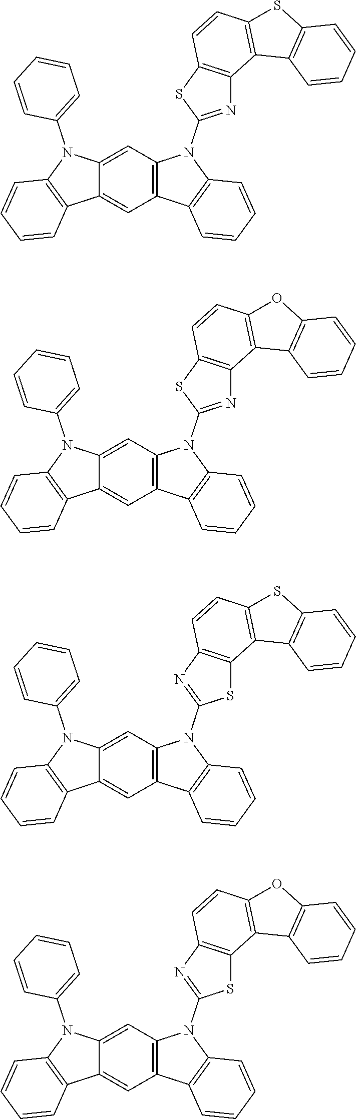 [SMILES: c1ccc(-n2c3ccccc3c3cc4c5ccccc5n(-c5nc6c(ccc7sc8ccccc8c76)s5)c4cc32)cc1 ; c1ccc(-n2c3ccccc3c3cc4c5ccccc5n(-c5nc6c(ccc7oc8ccccc8c76)s5)c4cc32)cc1 ; c1ccc(-n2c3ccccc3c3cc4c5ccccc5n(-c5nc6ccc7sc8ccccc8c7c6s5)c4cc32)cc1 ; c1ccc(-n2c3ccccc3c3cc4c5ccccc5n(-c5nc6ccc7oc8ccccc8c7c6s5)c4cc32)cc1]

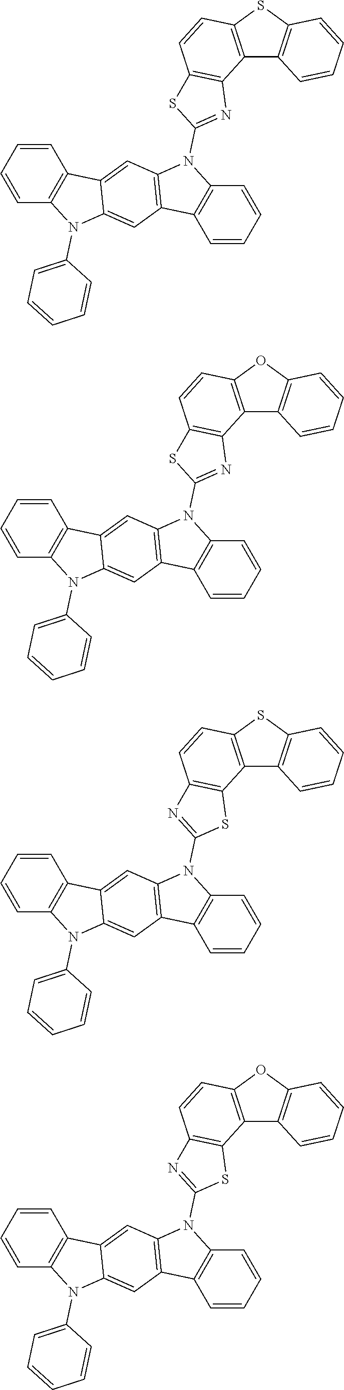 [SMILES: c1ccc(-n2c3ccccc3c3cc4c(cc32)c2ccccc2n4-c2nc3c(ccc4sc5ccccc5c43)s2)cc1 ; c1ccc(-n2c3ccccc3c3cc4c(cc32)c2ccccc2n4-c2nc3c(ccc4oc5ccccc5c43)s2)cc1 ; c1ccc(-n2c3ccccc3c3cc4c(cc32)c2ccccc2n4-c2nc3ccc4sc5ccccc5c4c3s2)cc1 ; c1ccc(-n2c3ccccc3c3cc4c(cc32)c2ccccc2n4-c2nc3ccc4oc5ccccc5c4c3s2)cc1]

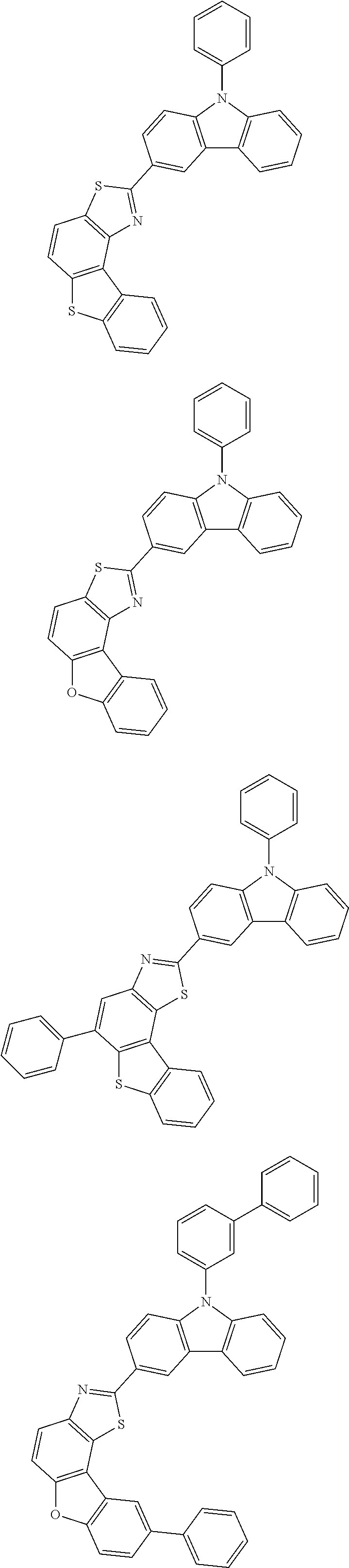 [SMILES: c1ccc(-n2c3ccccc3c3cc(-c4nc5c(ccc6sc7ccccc7c65)s4)ccc32)cc1 ; c1ccc(-n2c3ccccc3c3cc(-c4nc5c(ccc6oc7ccccc7c65)s4)ccc32)cc1 ; c1ccc(-c2cc3nc(-c4ccc5c(c4)c4ccccc4n5-c4ccccc4)sc3c3c2sc2ccccc23)cc1 ; c1ccc(-c2cccc(-n3c4ccccc4c4cc(-c5nc6ccc7oc8ccc(-c9ccccc9)cc8c7c6s5)ccc43)c2)cc1]

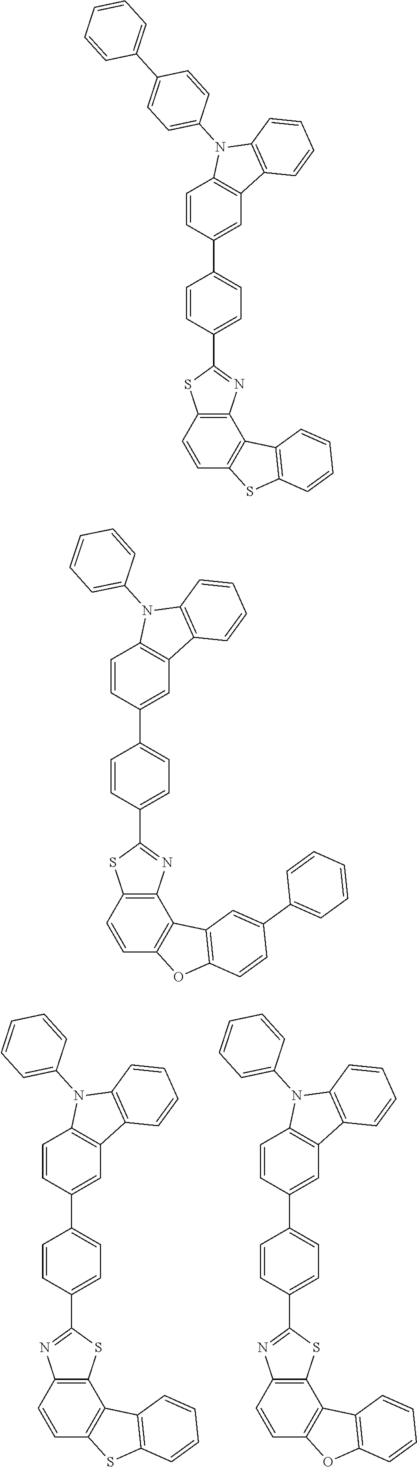

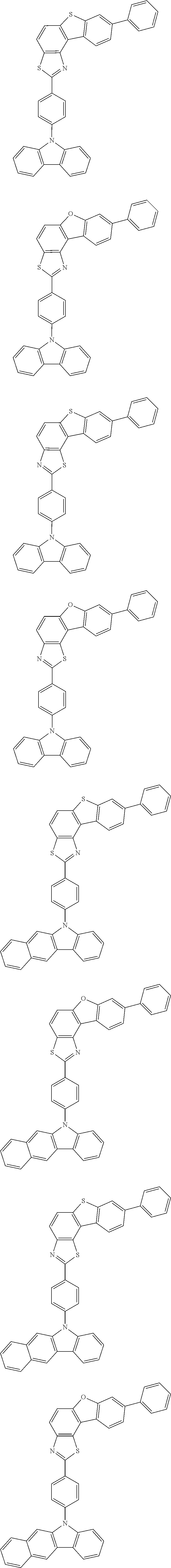

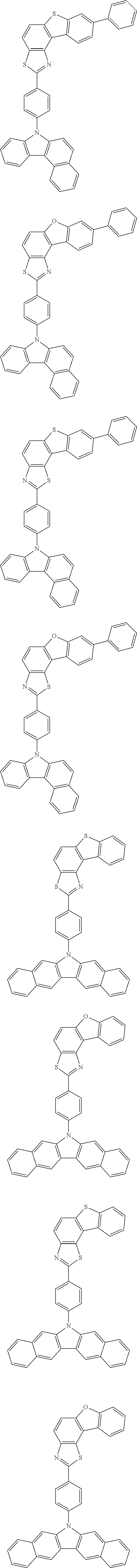

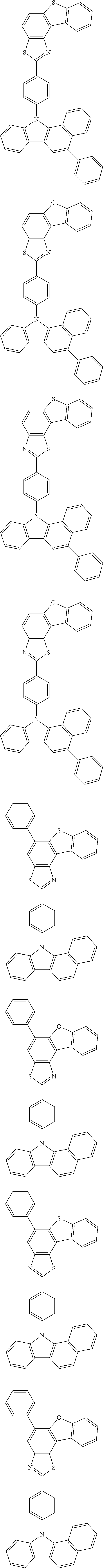

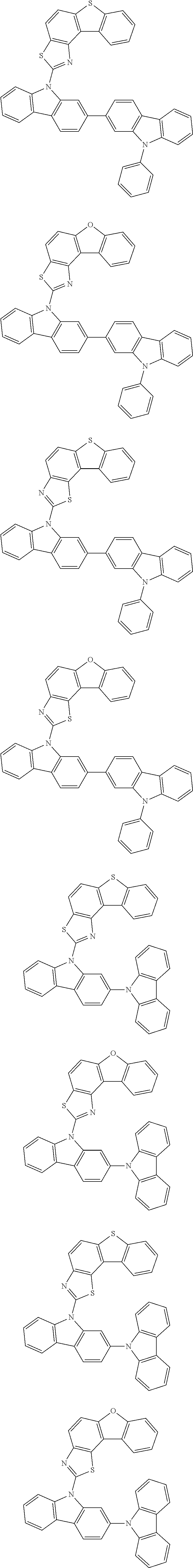

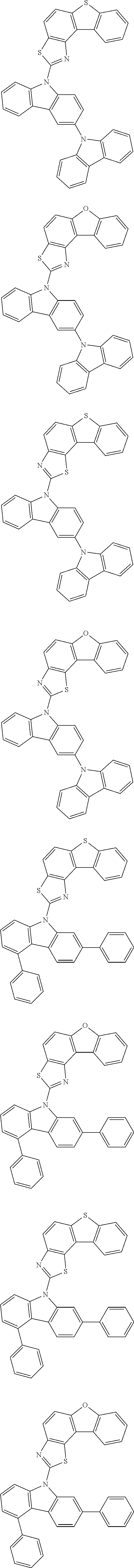

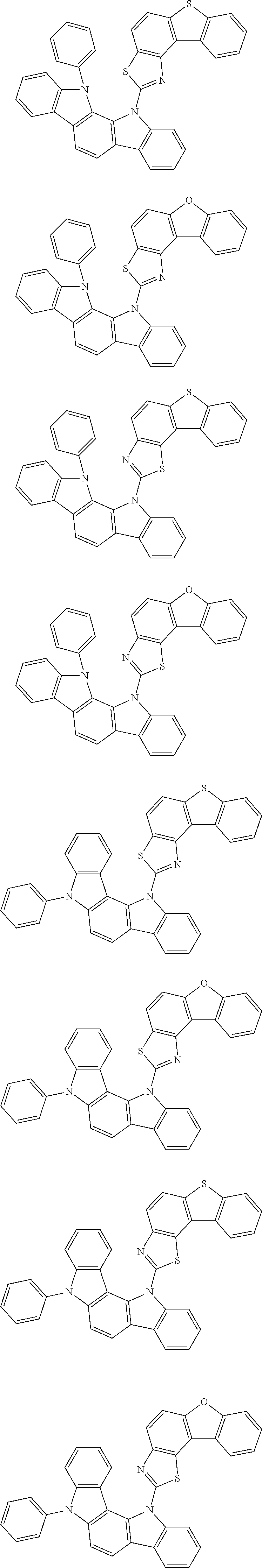

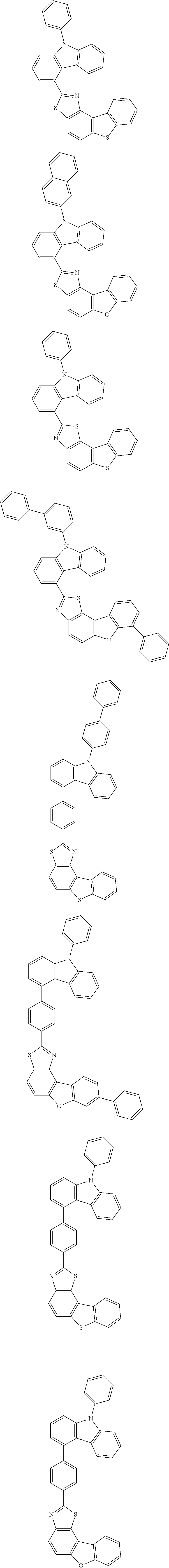

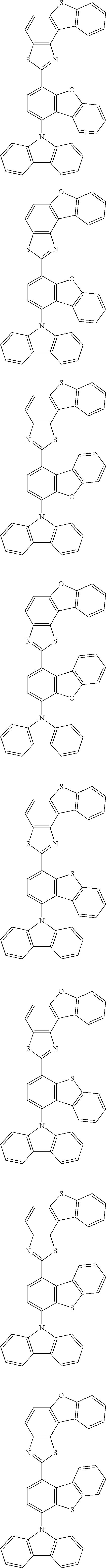

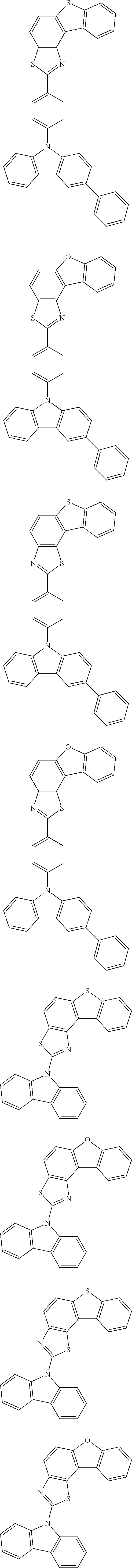

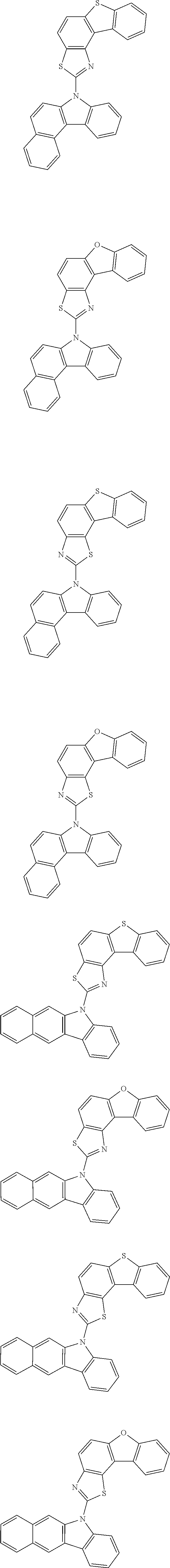

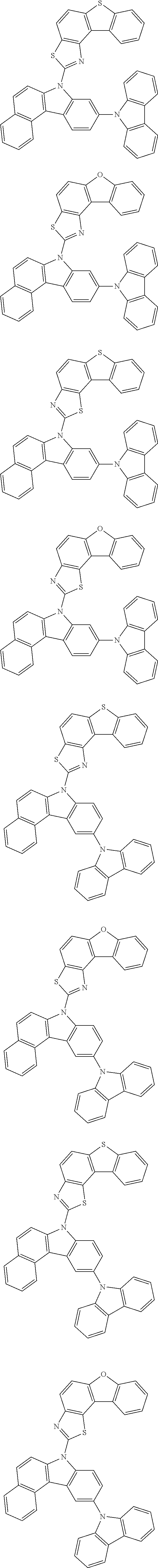

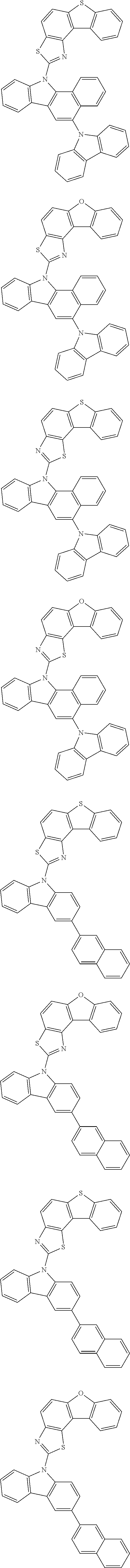

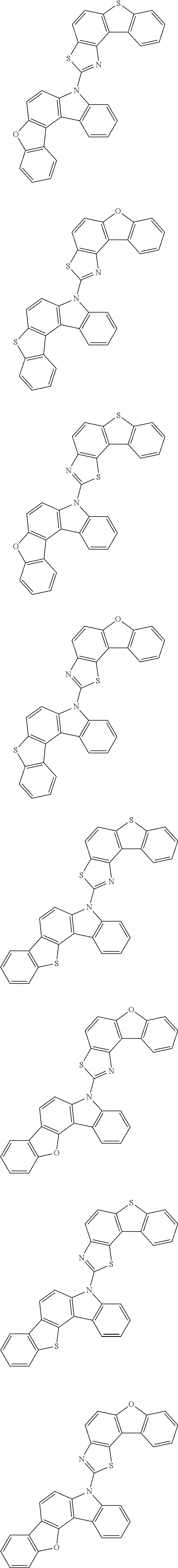

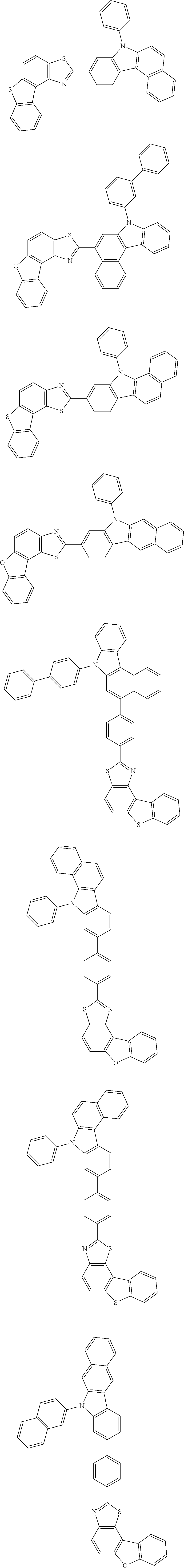

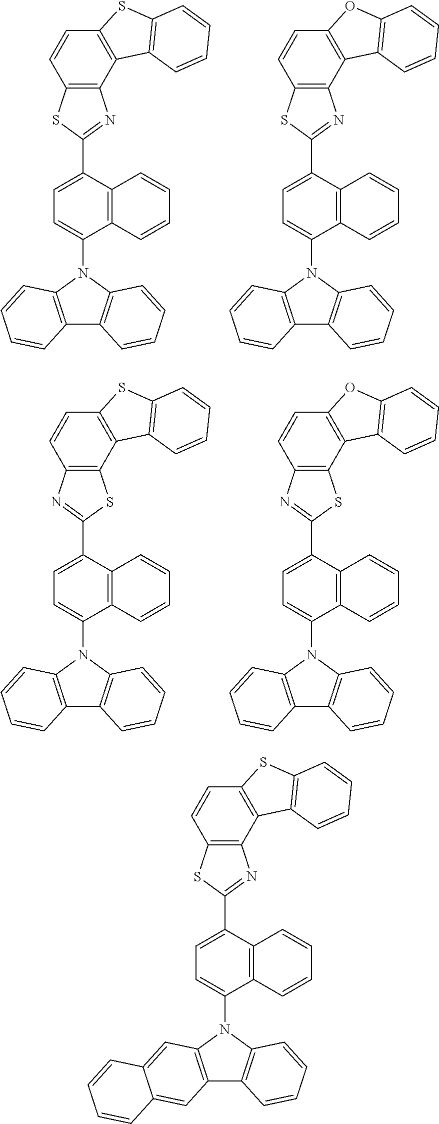

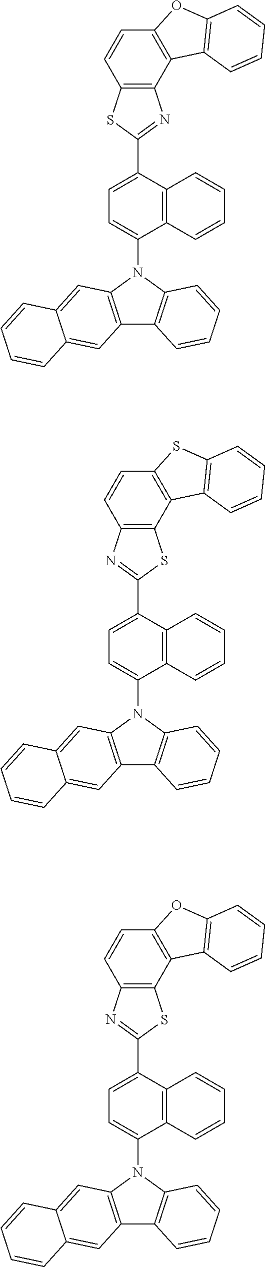 [SMILES: c1ccc2cc3c(cc2c1)c1ccccc1n3-c1ccc(-c2nc3c(ccc4oc5ccccc5c43)s2)c2ccccc12 ; c1ccc2cc3c(cc2c1)c1ccccc1n3-c1ccc(-c2nc3ccc4sc5ccccc5c4c3s2)c2ccccc12 ; c1ccc2cc3c(cc2c1)c1ccccc1n3-c1ccc(-c2nc3ccc4oc5ccccc5c4c3s2)c2ccccc12]

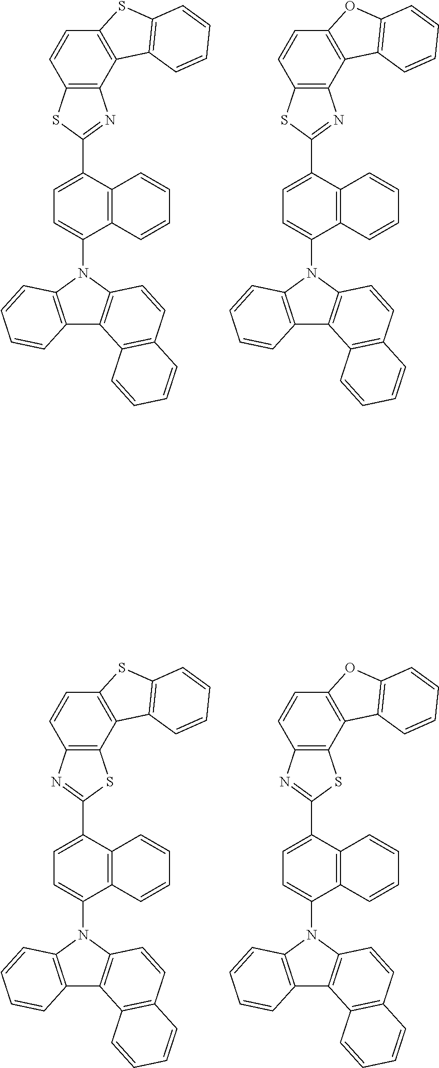 [SMILES: c1ccc2c(c1)ccc1c2c2ccccc2n1-c1ccc(-c2nc3c(ccc4sc5ccccc5c43)s2)c2ccccc12 ; c1ccc2c(c1)ccc1c2c2ccccc2n1-c1ccc(-c2nc3c(ccc4oc5ccccc5c43)s2)c2ccccc12 ; c1ccc2c(c1)ccc1c2c2ccccc2n1-c1ccc(-c2nc3ccc4sc5ccccc5c4c3s2)c2ccccc12 ; c1ccc2c(c1)ccc1c2c2ccccc2n1-c1ccc(-c2nc3ccc4oc5ccccc5c4c3s2)c2ccccc12]

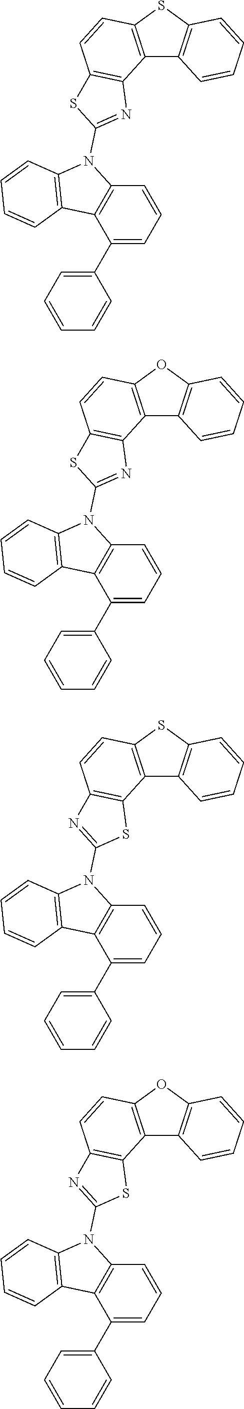 [SMILES: c1ccc(-c2cccc3c2c2ccccc2n3-c2nc3c(ccc4sc5ccccc5c43)s2)cc1 ; c1ccc(-c2cccc3c2c2ccccc2n3-c2nc3c(ccc4oc5ccccc5c43)s2)cc1 ; c1ccc(-c2cccc3c2c2ccccc2n3-c2nc3ccc4sc5ccccc5c4c3s2)cc1 ; c1ccc(-c2cccc3c2c2ccccc2n3-c2nc3ccc4oc5ccccc5c4c3s2)cc1]

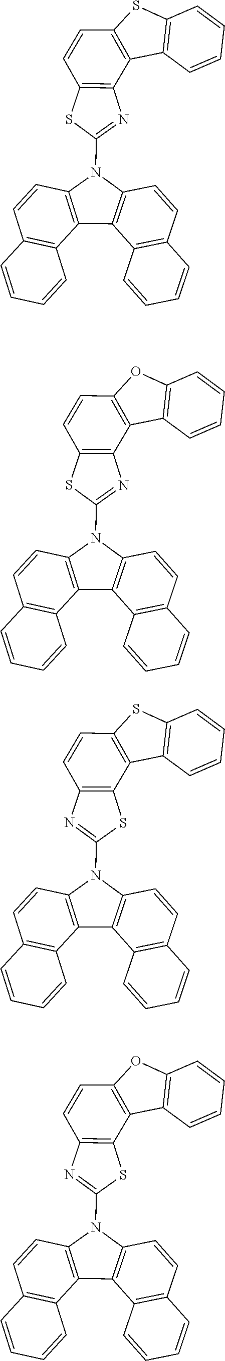

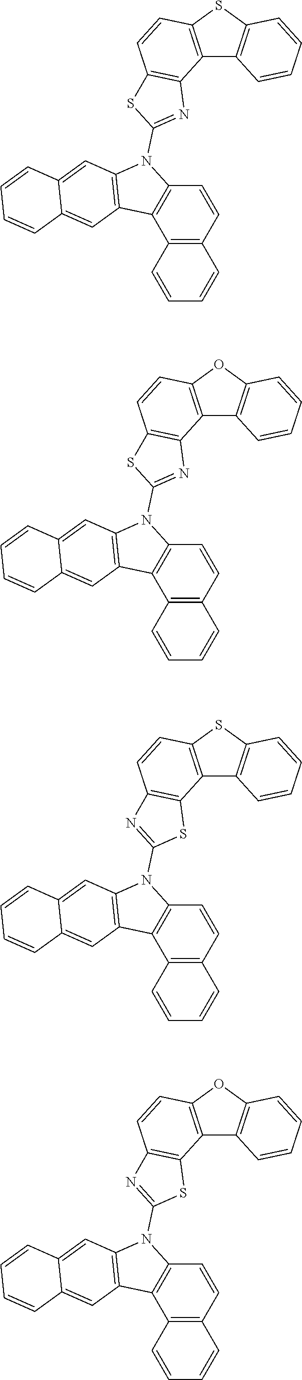 [SMILES: c1ccc2cc3c(cc2c1)c1c2ccccc2ccc1n3-c1nc2c(ccc3sc4ccccc4c32)s1 ; c1ccc2cc3c(cc2c1)c1c2ccccc2ccc1n3-c1nc2c(ccc3oc4ccccc4c32)s1 ; c1ccc2cc3c(cc2c1)c1c2ccccc2ccc1n3-c1nc2ccc3sc4ccccc4c3c2s1 ; c1ccc2cc3c(cc2c1)c1c2ccccc2ccc1n3-c1nc2ccc3oc4ccccc4c3c2s1]

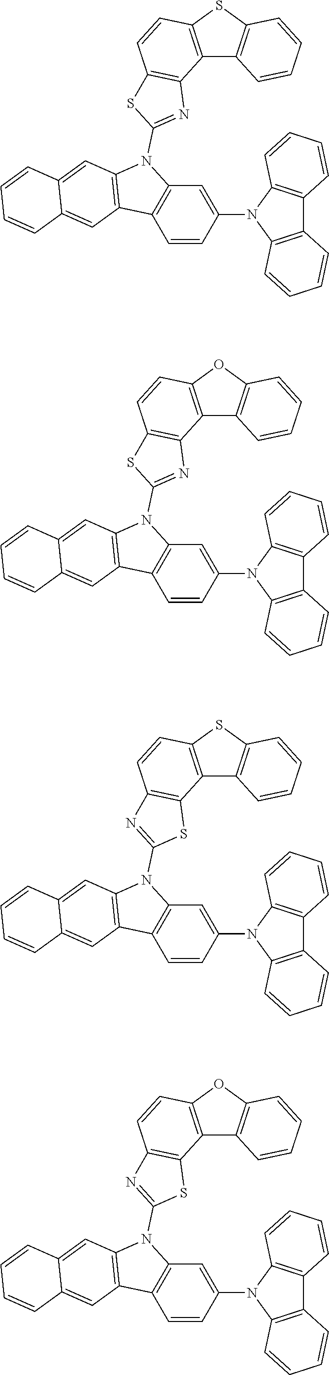 [SMILES: c1ccc2cc3c(cc2c1)c1ccc(-n2c4ccccc4c4ccccc42)cc1n3-c1nc2c(ccc3sc4ccccc4c32)s1 ; c1ccc2cc3c(cc2c1)c1ccc(-n2c4ccccc4c4ccccc42)cc1n3-c1nc2c(ccc3oc4ccccc4c32)s1 ; c1ccc2cc3c(cc2c1)c1ccc(-n2c4ccccc4c4ccccc42)cc1n3-c1nc2ccc3sc4ccccc4c3c2s1 ; c1ccc2cc3c(cc2c1)c1ccc(-n2c4ccccc4c4ccccc42)cc1n3-c1nc2ccc3oc4ccccc4c3c2s1]

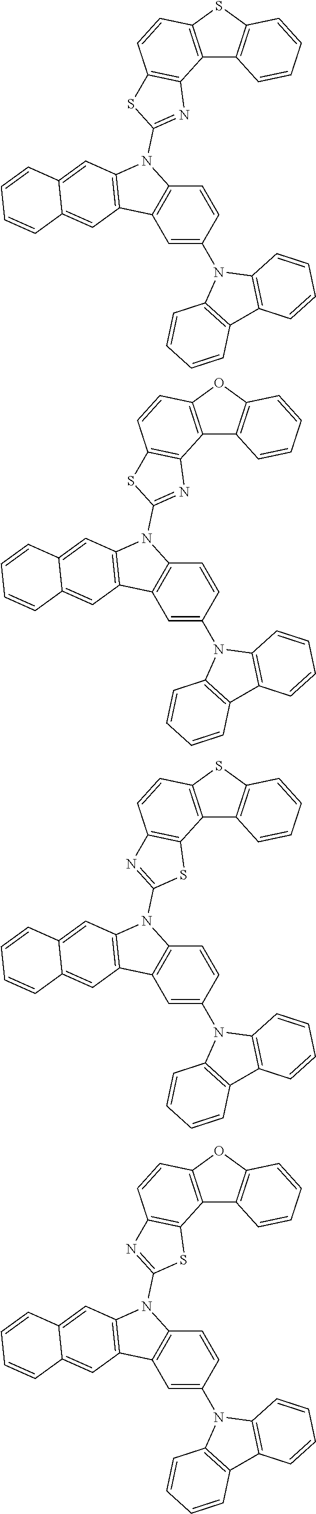

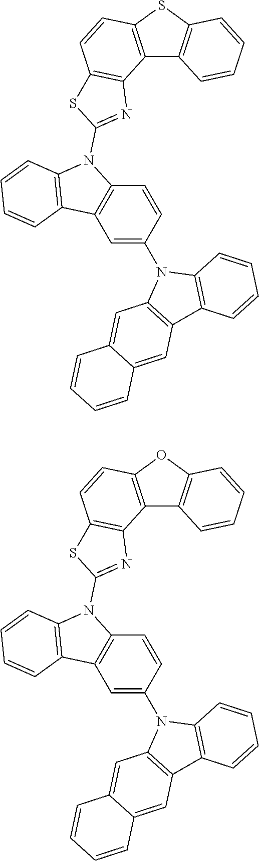 [SMILES: c1ccc2cc3c(cc2c1)c1ccccc1n3-c1ccc2c(c1)c1ccccc1n2-c1nc2c(ccc3sc4ccccc4c32)s1 ; c1ccc2cc3c(cc2c1)c1ccccc1n3-c1ccc2c(c1)c1ccccc1n2-c1nc2c(ccc3oc4ccccc4c32)s1]

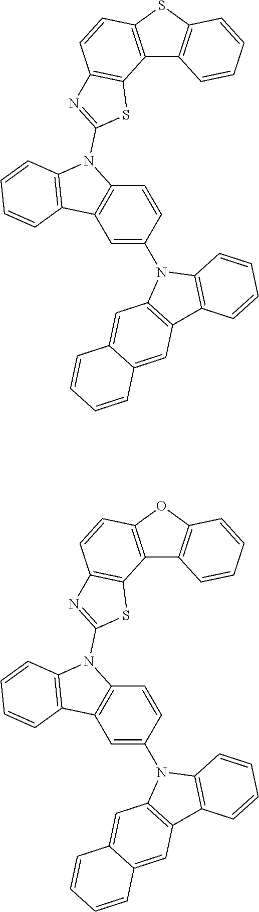 [SMILES: c1ccc2cc3c(cc2c1)c1ccccc1n3-c1ccc2c(c1)c1ccccc1n2-c1nc2ccc3sc4ccccc4c3c2s1 ; c1ccc2cc3c(cc2c1)c1ccccc1n3-c1ccc2c(c1)c1ccccc1n2-c1nc2ccc3oc4ccccc4c3c2s1]

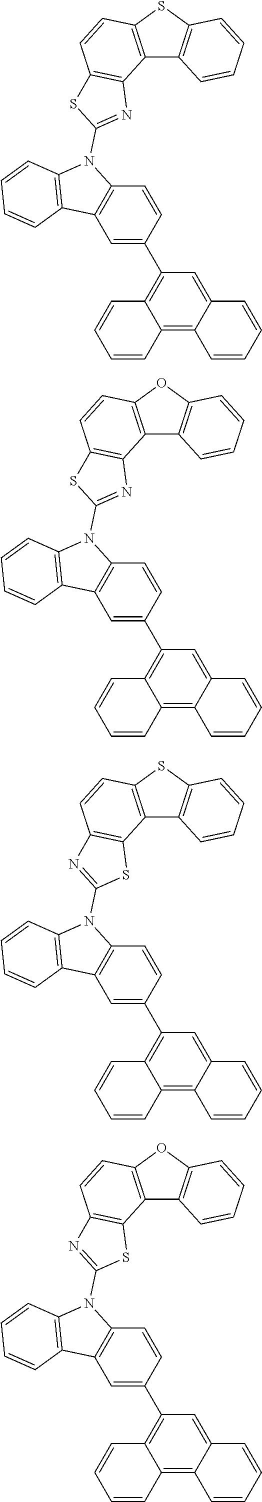

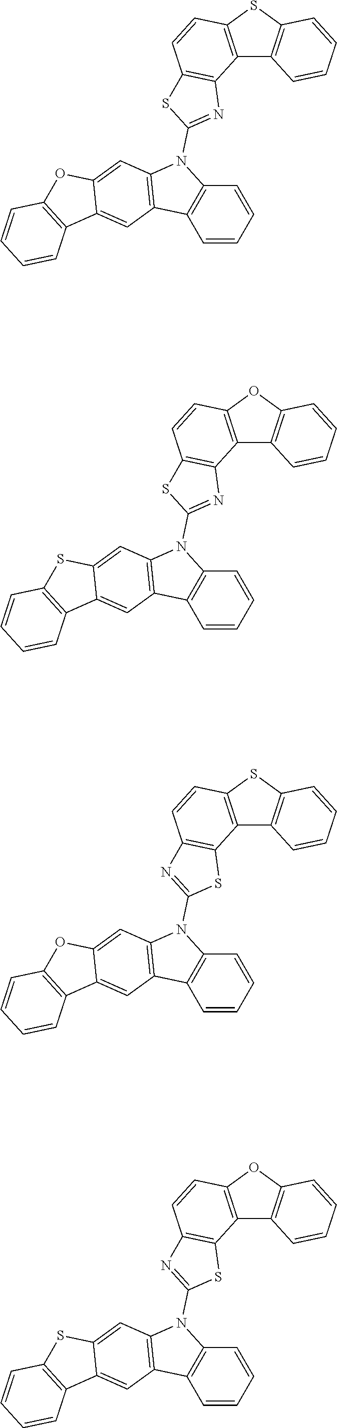 [SMILES: c1ccc2c(c1)oc1cc3c(cc12)c1ccccc1n3-c1nc2c(ccc3sc4ccccc4c32)s1 ; c1ccc2c(c1)oc1ccc3sc(-n4c5ccccc5c5cc6c(cc54)sc4ccccc46)nc3c12 ; c1ccc2c(c1)oc1cc3c(cc12)c1ccccc1n3-c1nc2ccc3sc4ccccc4c3c2s1 ; c1ccc2c(c1)oc1ccc3nc(-n4c5ccccc5c5cc6c(cc54)sc4ccccc46)sc3c12]

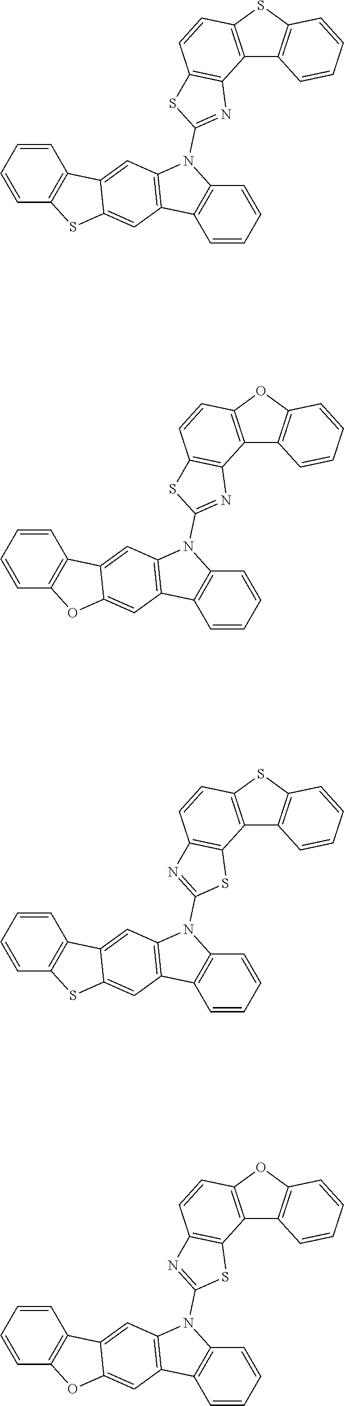 [SMILES: c1ccc2c(c1)sc1cc3c4ccccc4n(-c4nc5c(ccc6sc7ccccc7c65)s4)c3cc12 ; c1ccc2c(c1)oc1cc3c4ccccc4n(-c4nc5c(ccc6oc7ccccc7c65)s4)c3cc12 ; c1ccc2c(c1)sc1cc3c4ccccc4n(-c4nc5ccc6sc7ccccc7c6c5s4)c3cc12 ; c1ccc2c(c1)oc1cc3c4ccccc4n(-c4nc5ccc6oc7ccccc7c6c5s4)c3cc12]

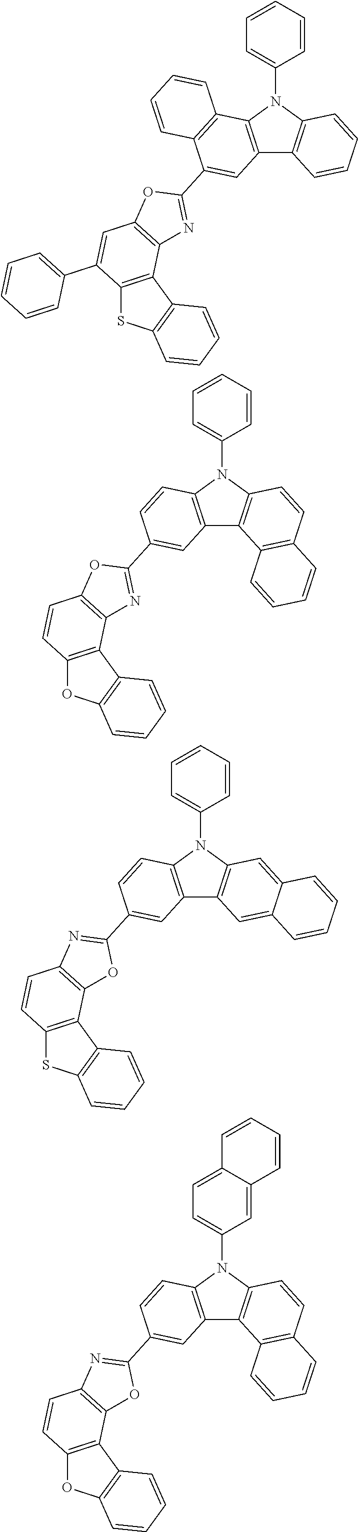

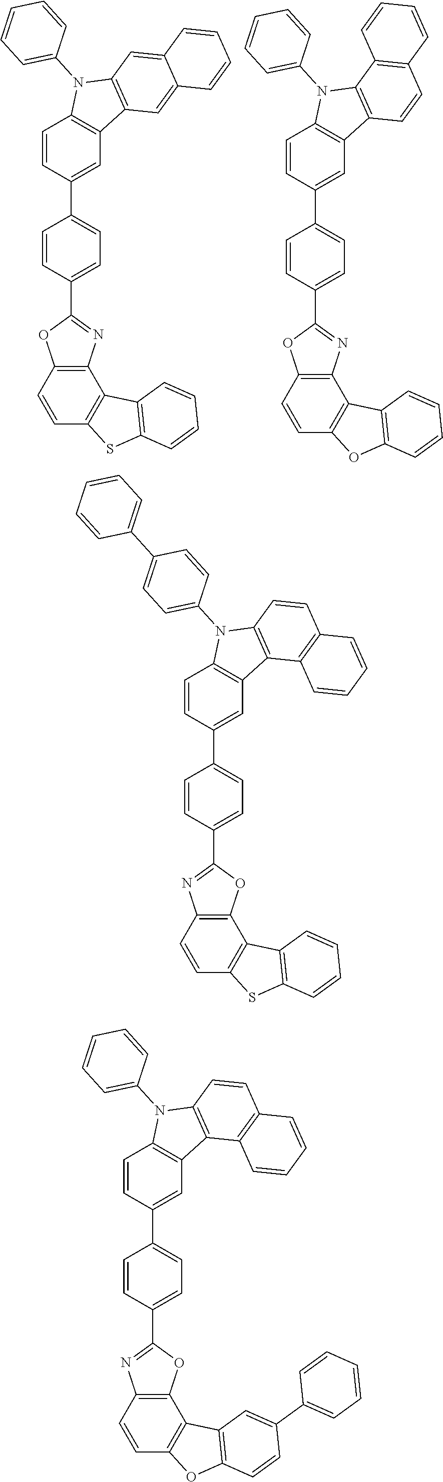

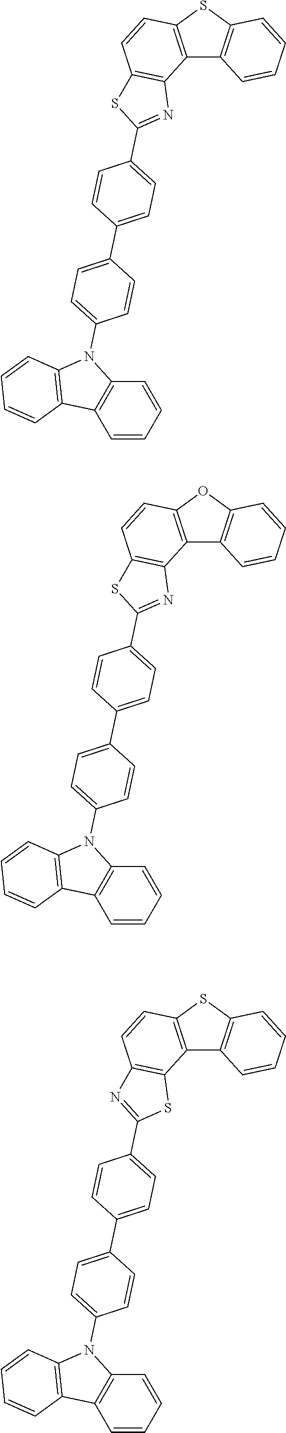 [SMILES: c1ccc2c(c1)sc1ccc3sc(-c4ccc(-c5ccc(-n6c7ccccc7c7ccccc76)cc5)cc4)nc3c12 ; c1ccc2c(c1)oc1ccc3sc(-c4ccc(-c5ccc(-n6c7ccccc7c7ccccc76)cc5)cc4)nc3c12 ; c1ccc2c(c1)sc1ccc3nc(-c4ccc(-c5ccc(-n6c7ccccc7c7ccccc76)cc5)cc4)sc3c12]

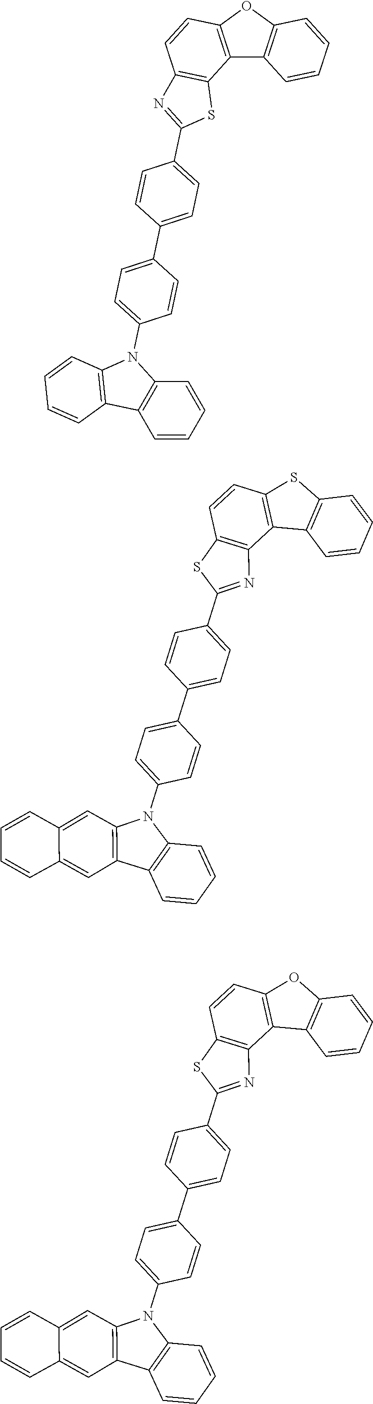 [SMILES: c1ccc2c(c1)oc1ccc3nc(-c4ccc(-c5ccc(-n6c7ccccc7c7ccccc76)cc5)cc4)sc3c12 ; c1ccc2cc3c(cc2c1)c1ccccc1n3-c1ccc(-c2ccc(-c3nc4c(ccc5sc6ccccc6c54)s3)cc2)cc1 ; c1ccc2cc3c(cc2c1)c1ccccc1n3-c1ccc(-c2ccc(-c3nc4c(ccc5oc6ccccc6c54)s3)cc2)cc1]

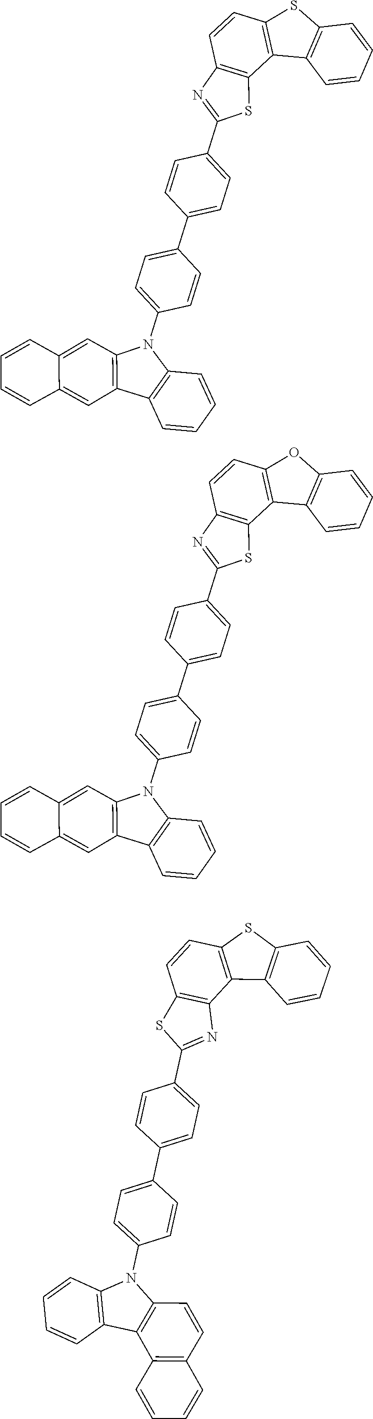 [SMILES: c1ccc2cc3c(cc2c1)c1ccccc1n3-c1ccc(-c2ccc(-c3nc4ccc5sc6ccccc6c5c4s3)cc2)cc1 ; c1ccc2cc3c(cc2c1)c1ccccc1n3-c1ccc(-c2ccc(-c3nc4ccc5oc6ccccc6c5c4s3)cc2)cc1 ; c1ccc2c(c1)ccc1c2c2ccccc2n1-c1ccc(-c2ccc(-c3nc4c(ccc5sc6ccccc6c54)s3)cc2)cc1]

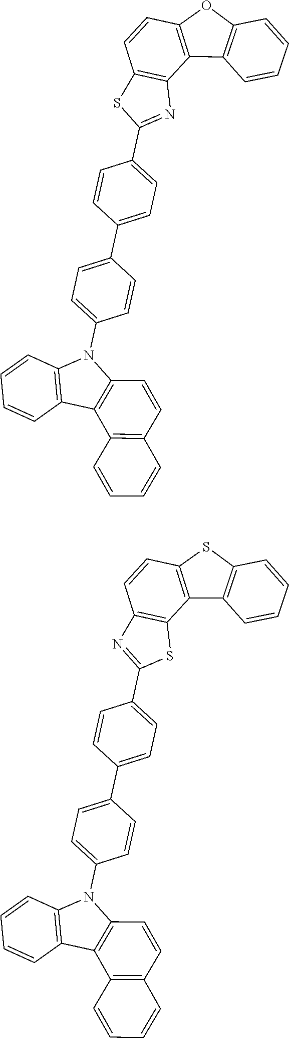 [SMILES: c1ccc2c(c1)ccc1c2c2ccccc2n1-c1ccc(-c2ccc(-c3nc4c(ccc5oc6ccccc6c54)s3)cc2)cc1 ; c1ccc2c(c1)ccc1c2c2ccccc2n1-c1ccc(-c2ccc(-c3nc4ccc5sc6ccccc6c5c4s3)cc2)cc1]

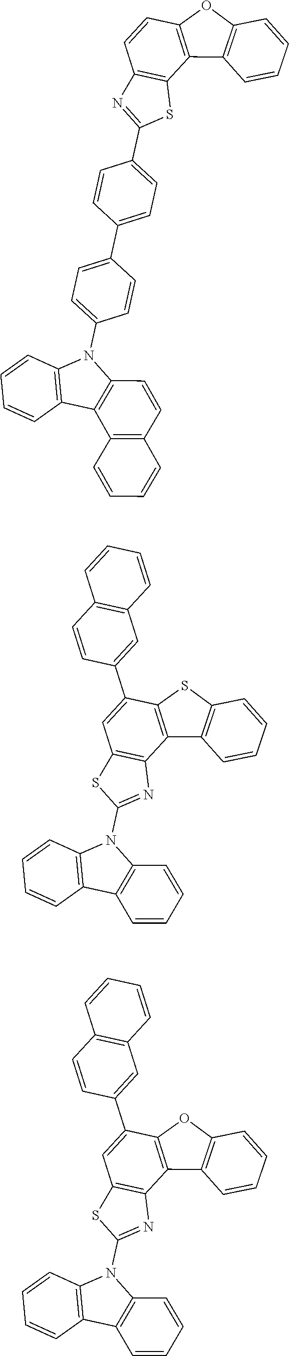 [SMILES: c1ccc2c(c1)ccc1c2c2ccccc2n1-c1ccc(-c2ccc(-c3nc4ccc5oc6ccccc6c5c4s3)cc2)cc1 ; c1ccc2cc(-c3cc4sc(-n5c6ccccc6c6ccccc65)nc4c4c3sc3ccccc34)ccc2c1 ; c1ccc2cc(-c3cc4sc(-n5c6ccccc6c6ccccc65)nc4c4c3oc3ccccc34)ccc2c1]

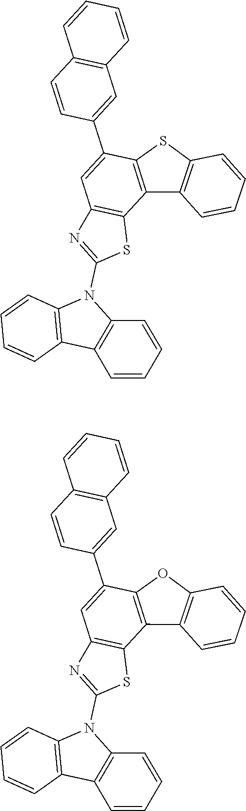 [SMILES: c1ccc2cc(-c3cc4nc(-n5c6ccccc6c6ccccc65)sc4c4c3sc3ccccc34)ccc2c1 ; c1ccc2cc(-c3cc4nc(-n5c6ccccc6c6ccccc65)sc4c4c3oc3ccccc34)ccc2c1]

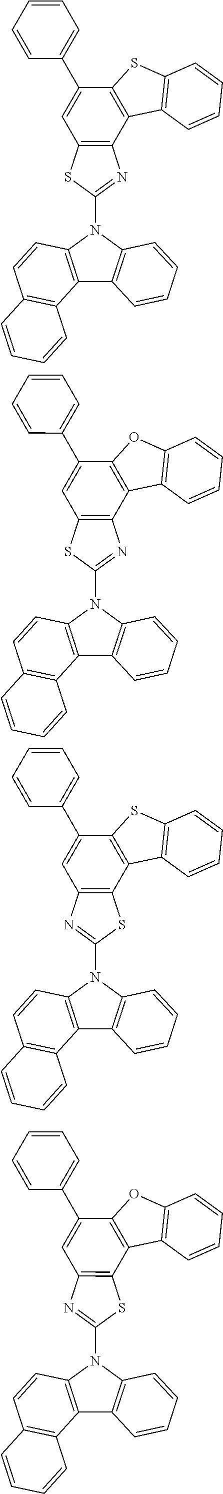

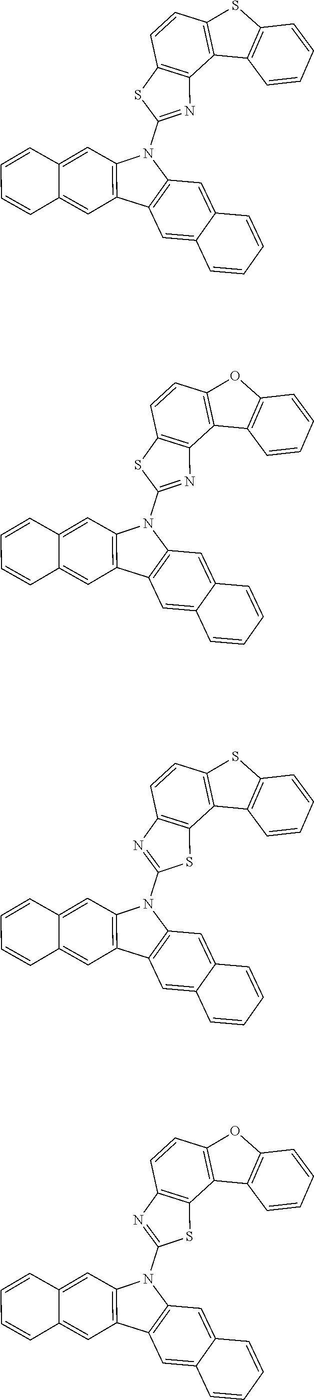 [SMILES: c1ccc2cc3c(cc2c1)c1cc2ccccc2cc1n3-c1nc2c(ccc3sc4ccccc4c32)s1 ; c1ccc2cc3c(cc2c1)c1cc2ccccc2cc1n3-c1nc2c(ccc3oc4ccccc4c32)s1 ; c1ccc2cc3c(cc2c1)c1cc2ccccc2cc1n3-c1nc2ccc3sc4ccccc4c3c2s1 ; c1ccc2cc3c(cc2c1)c1cc2ccccc2cc1n3-c1nc2ccc3oc4ccccc4c3c2s1]

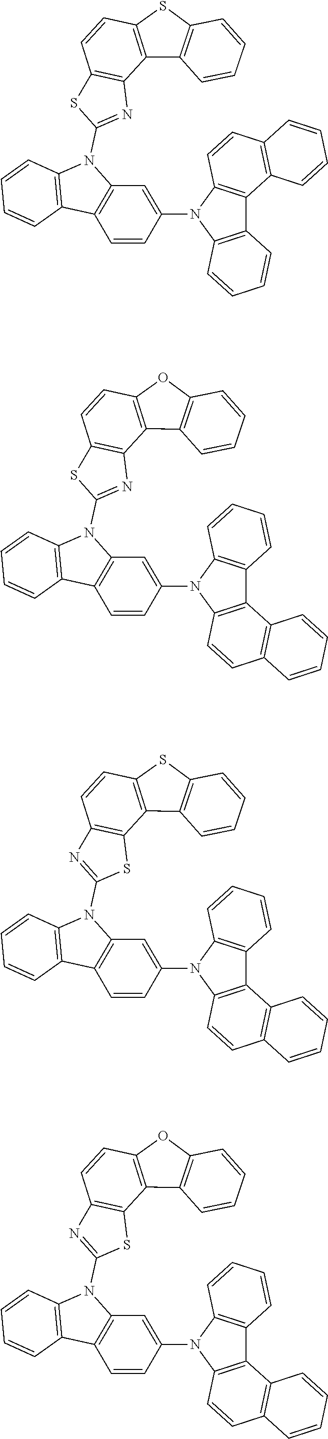 [SMILES: c1ccc2c(c1)ccc1c2c2ccccc2n1-c1ccc2c3ccccc3n(-c3nc4c(ccc5sc6ccccc6c54)s3)c2c1 ; c1ccc2c(c1)ccc1c2c2ccccc2n1-c1ccc2c3ccccc3n(-c3nc4c(ccc5oc6ccccc6c54)s3)c2c1 ; c1ccc2c(c1)ccc1c2c2ccccc2n1-c1ccc2c3ccccc3n(-c3nc4ccc5sc6ccccc6c5c4s3)c2c1 ; c1ccc2c(c1)ccc1c2c2ccccc2n1-c1ccc2c3ccccc3n(-c3nc4ccc5oc6ccccc6c5c4s3)c2c1]

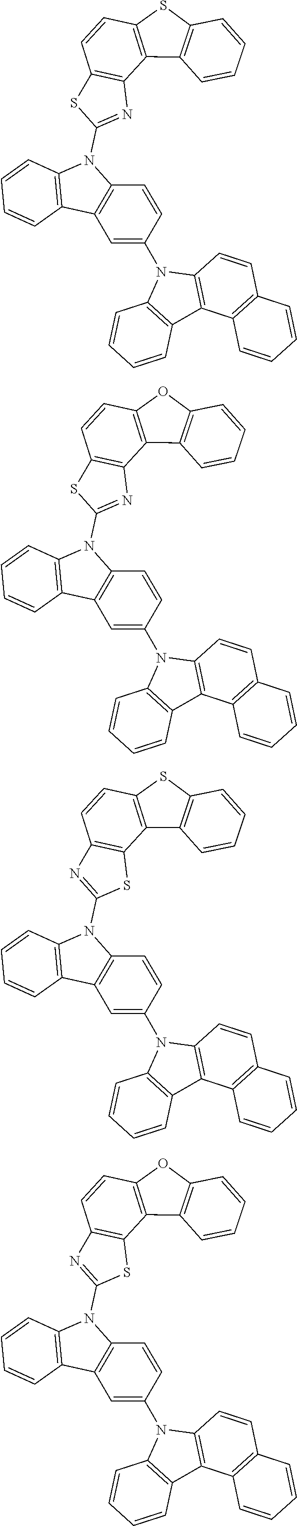

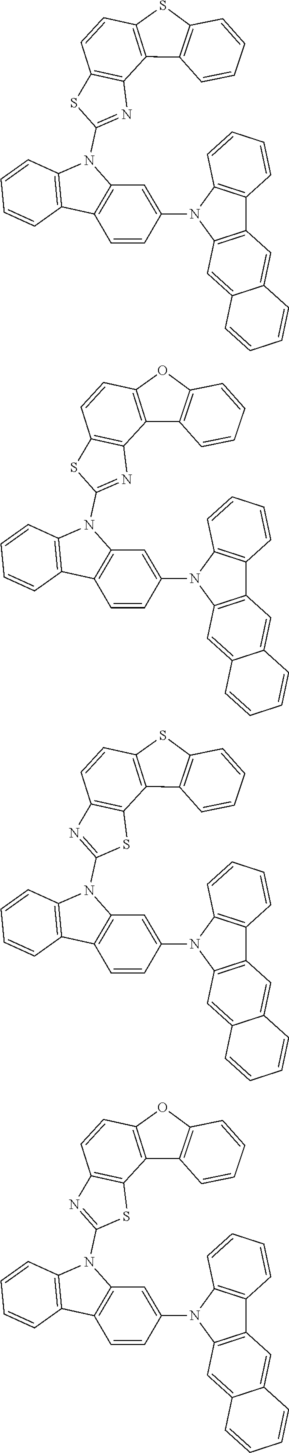

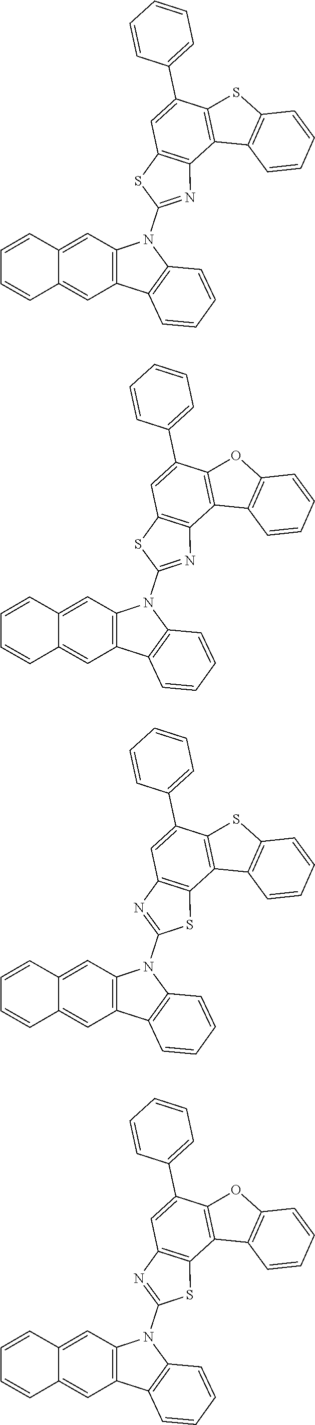

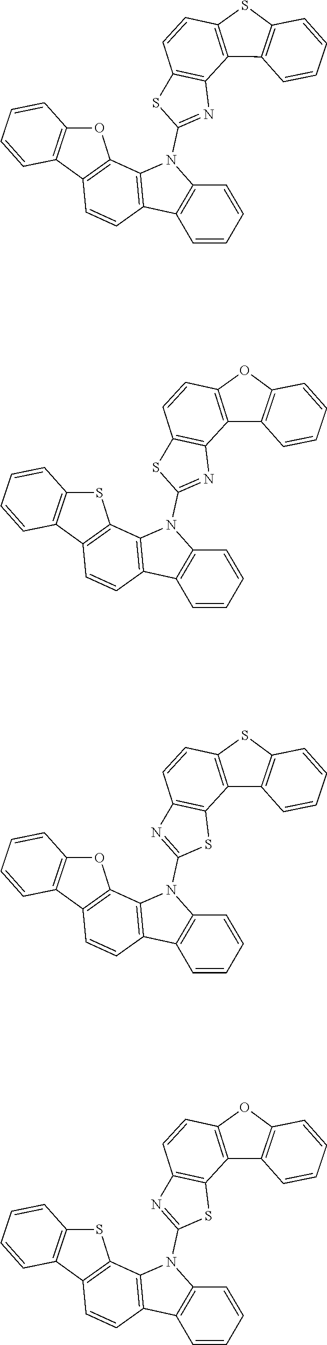 [SMILES: c1ccc2c(c1)oc1c2ccc2c3ccccc3n(-c3nc4c(ccc5sc6ccccc6c54)s3)c21 ; c1ccc2c(c1)oc1ccc3sc(-n4c5ccccc5c5ccc6c7ccccc7sc6c54)nc3c12 ; c1ccc2c(c1)oc1c2ccc2c3ccccc3n(-c3nc4ccc5sc6ccccc6c5c4s3)c21 ; c1ccc2c(c1)oc1ccc3nc(-n4c5ccccc5c5ccc6c7ccccc7sc6c54)sc3c12]

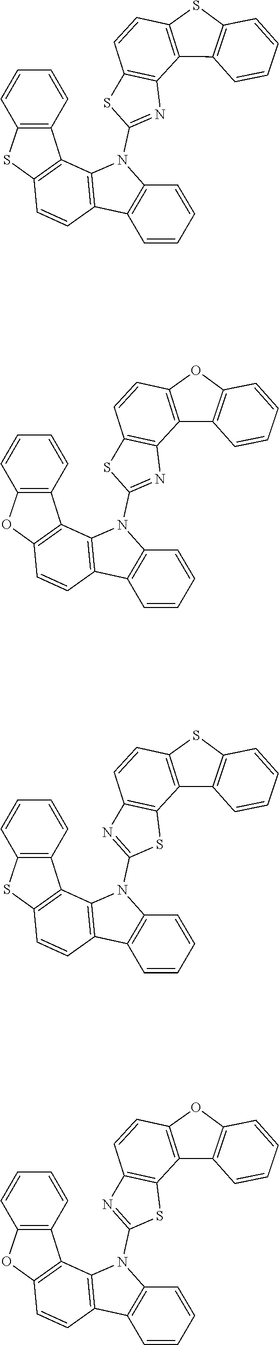 [SMILES: c1ccc2c(c1)sc1ccc3sc(-n4c5ccccc5c5ccc6sc7ccccc7c6c54)nc3c12 ; c1ccc2c(c1)oc1ccc3sc(-n4c5ccccc5c5ccc6oc7ccccc7c6c54)nc3c12 ; c1ccc2c(c1)sc1ccc3nc(-n4c5ccccc5c5ccc6sc7ccccc7c6c54)sc3c12 ; c1ccc2c(c1)oc1ccc3nc(-n4c5ccccc5c5ccc6oc7ccccc7c6c54)sc3c12]

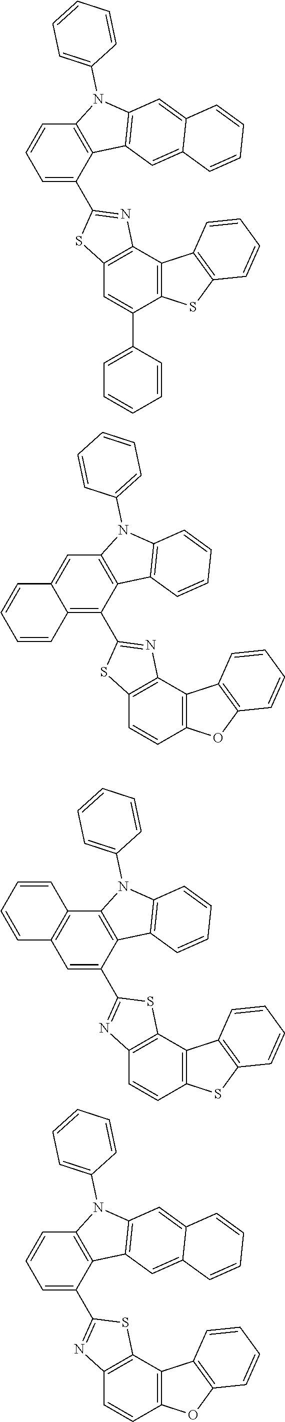

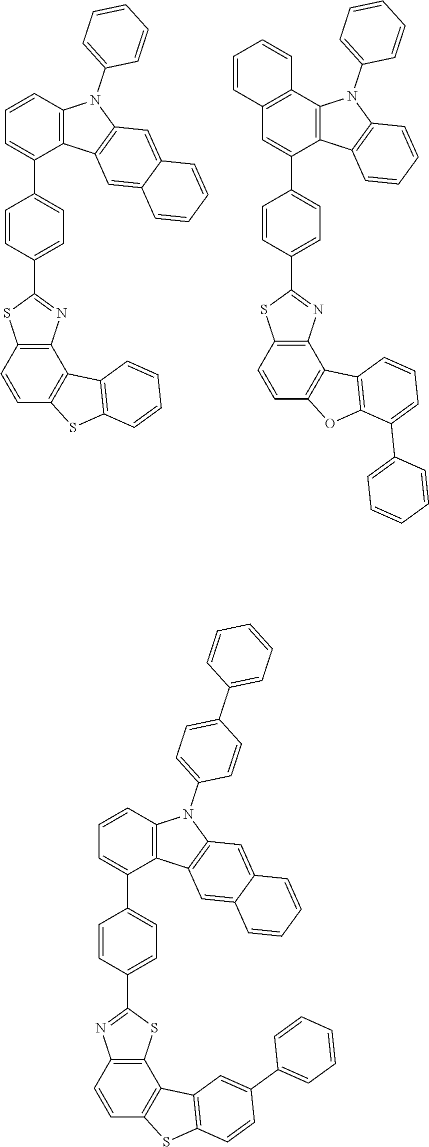 [SMILES: c1ccc(-n2c3cc4ccccc4cc3c3c(-c4ccc(-c5nc6c(ccc7sc8ccccc8c76)s5)cc4)cccc32)cc1 ; c1ccc(-c2cccc3c2oc2ccc4sc(-c5ccc(-c6cc7ccccc7c7c6c6ccccc6n7-c6ccccc6)cc5)nc4c23)cc1 ; c1ccc(-c2ccc(-n3c4cc5ccccc5cc4c4c(-c5ccc(-c6nc7ccc8sc9ccc(-c%10ccccc%10)cc9c8c7s6)cc5)cccc43)cc2)cc1]

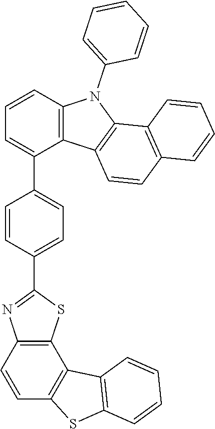 [SMILES: c1ccc(-n2c3cccc(-c4ccc(-c5nc6ccc7sc8ccccc8c7c6s5)cc4)c3c3ccc4ccccc4c32)cc1]

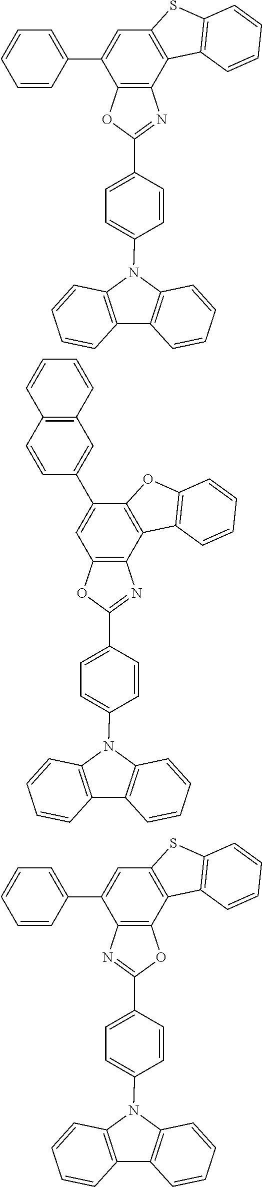

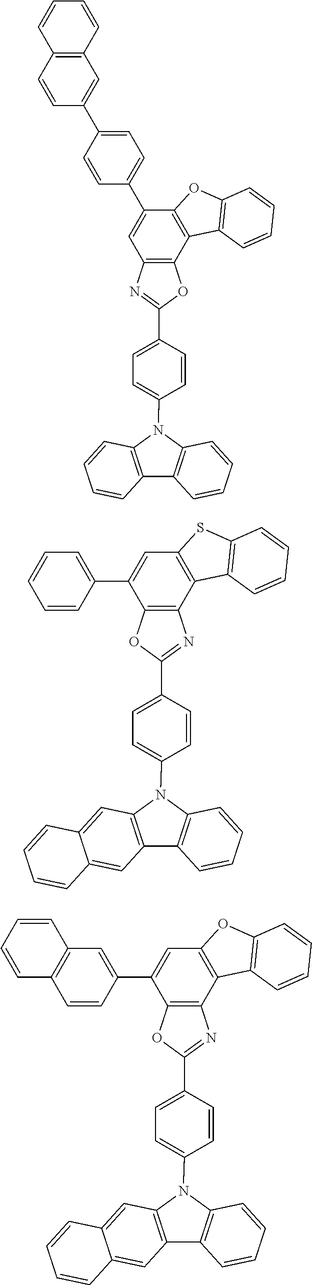

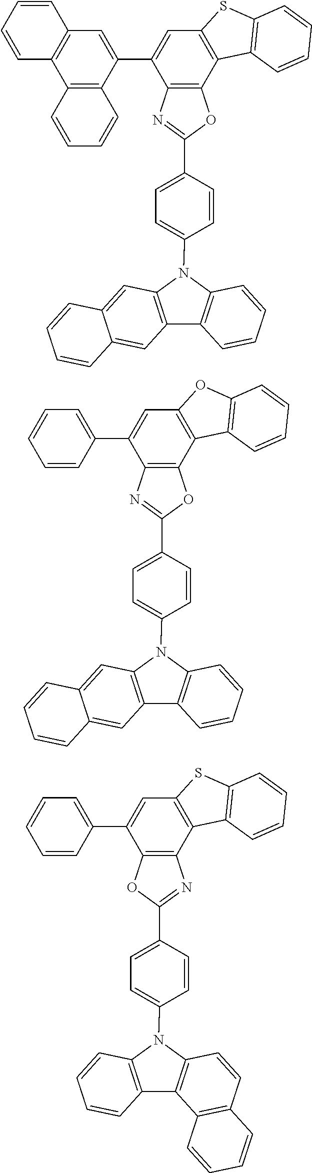

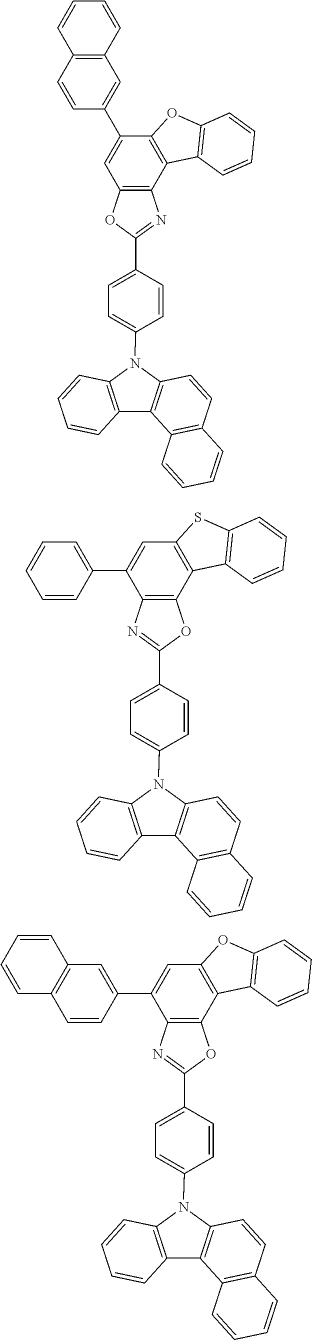

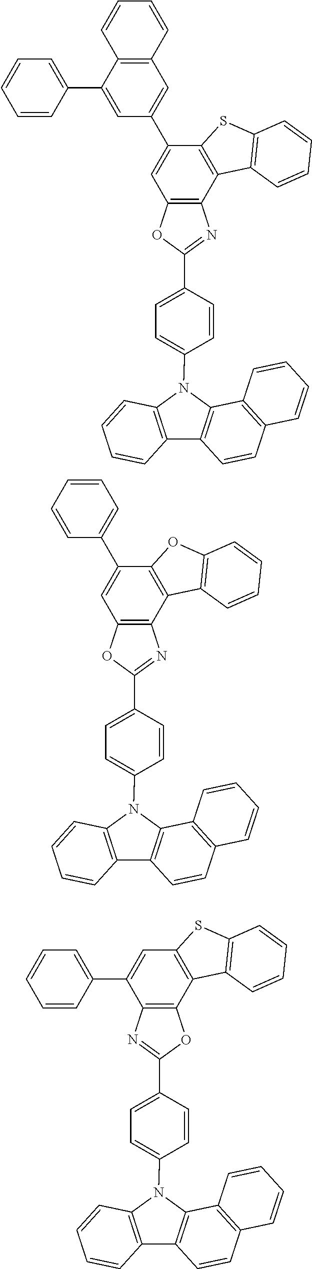

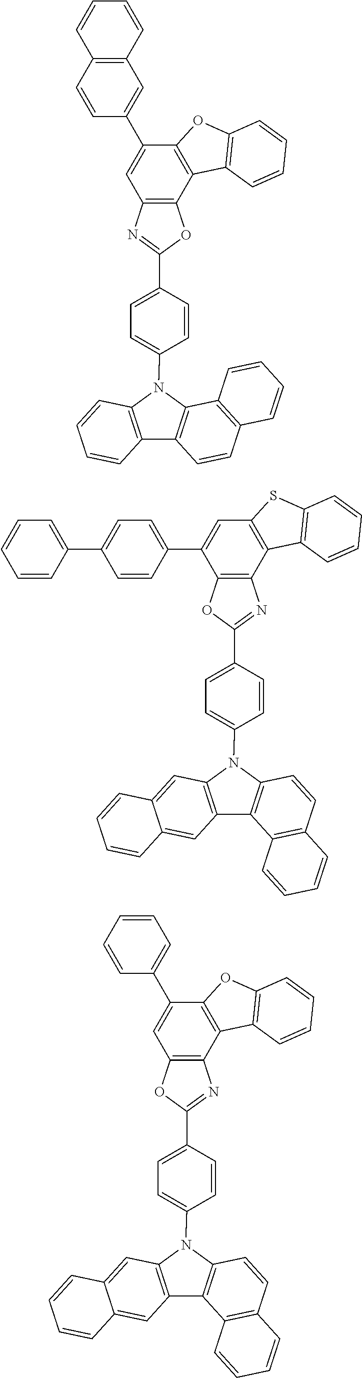 [SMILES: c1ccc2cc(-c3cc4nc(-c5ccc(-n6c7ccccc7c7ccc8ccccc8c76)cc5)oc4c4c3oc3ccccc34)ccc2c1 ; c1ccc(-c2ccc(-c3cc4sc5ccccc5c4c4nc(-c5ccc(-n6c7cc8ccccc8cc7c7c8ccccc8ccc76)cc5)oc34)cc2)cc1 ; c1ccc(-c2cc3oc(-c4ccc(-n5c6cc7ccccc7cc6c6c7ccccc7ccc65)cc4)nc3c3c2oc2ccccc23)cc1]

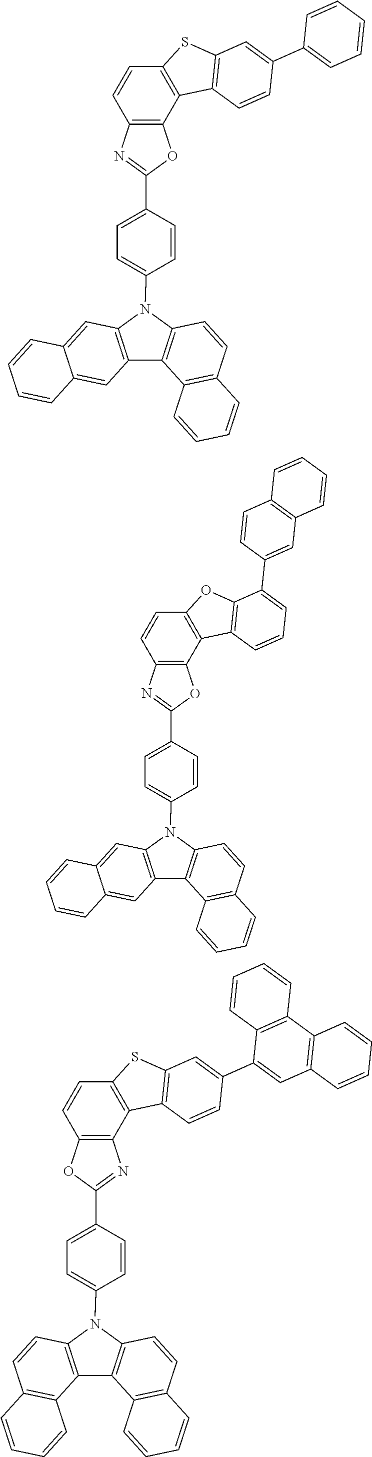

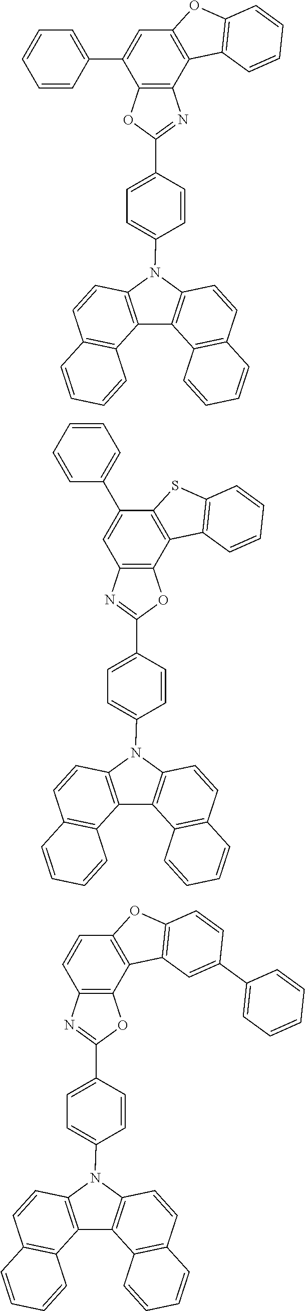

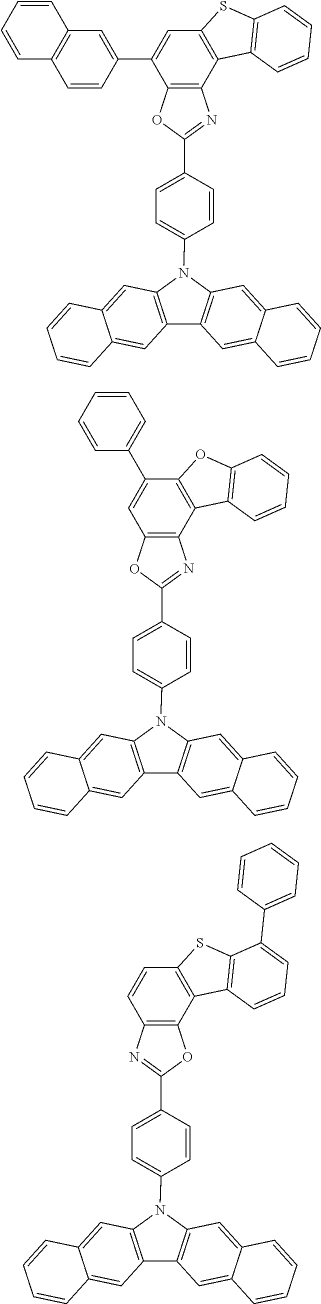

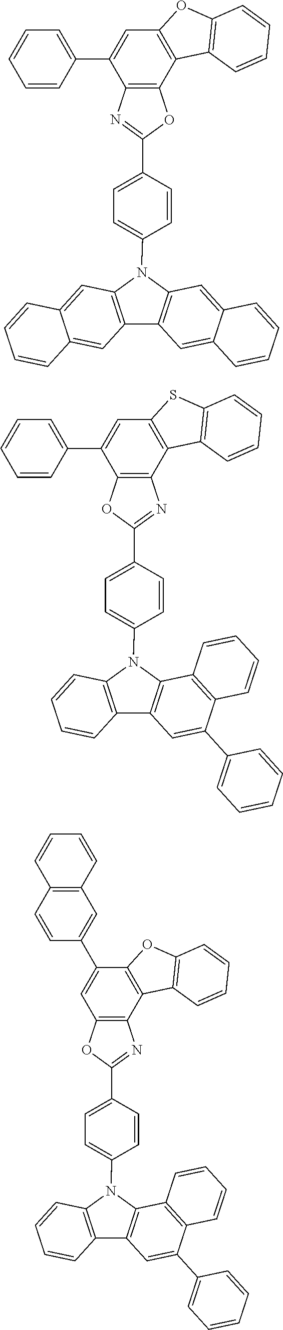

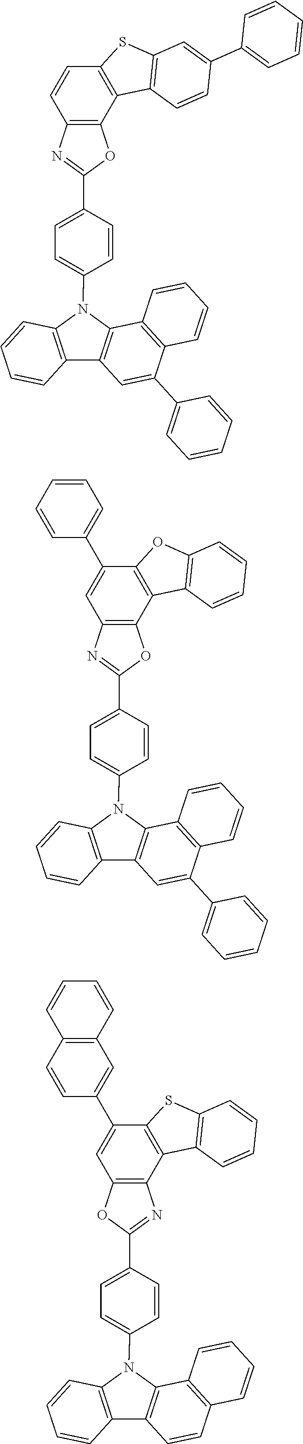 [SMILES: c1ccc(-c2ccc3c(c2)sc2ccc4nc(-c5ccc(-n6c7ccccc7c7cc(-c8ccccc8)c8ccccc8c76)cc5)oc4c23)cc1 ; c1ccc(-c2cc3c4ccccc4n(-c4ccc(-c5nc6cc(-c7ccccc7)c7oc8ccccc8c7c6o5)cc4)c3c3ccccc23)cc1 ; c1ccc2cc(-c3cc4oc(-c5ccc(-n6c7ccccc7c7ccc8ccccc8c76)cc5)nc4c4c3sc3ccccc34)ccc2c1]

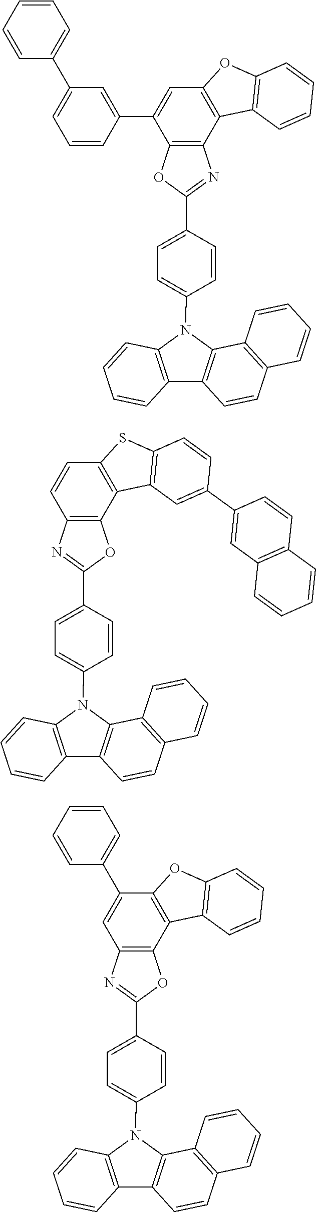

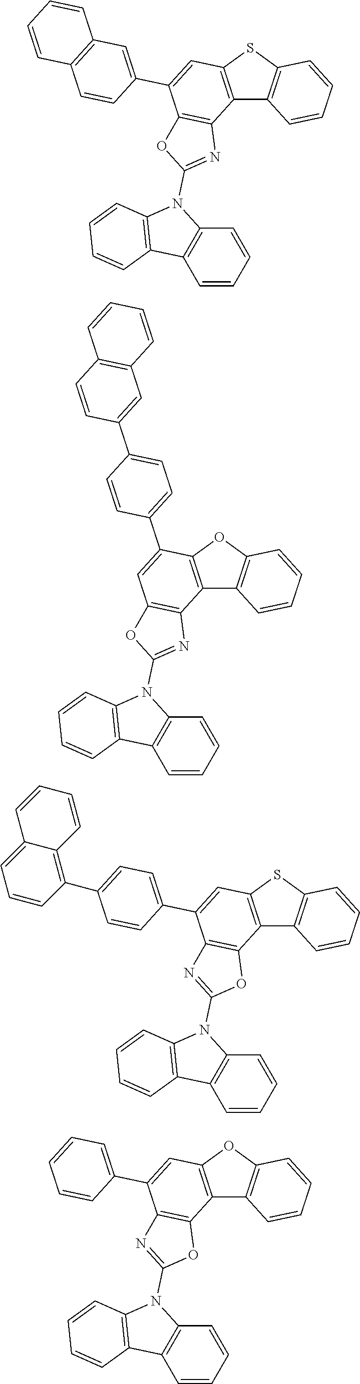

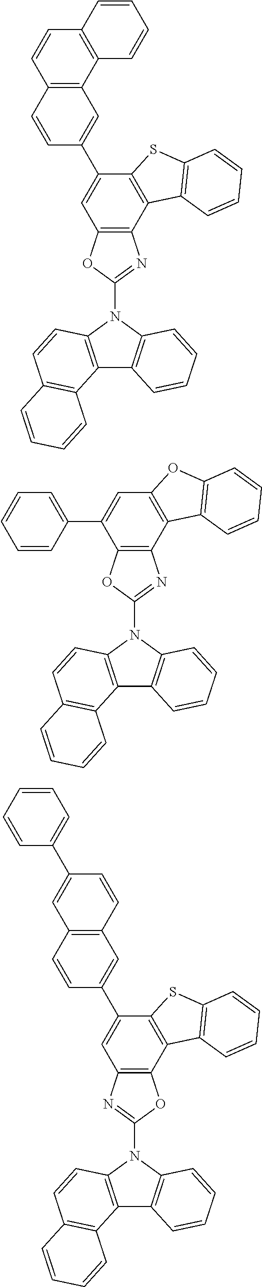

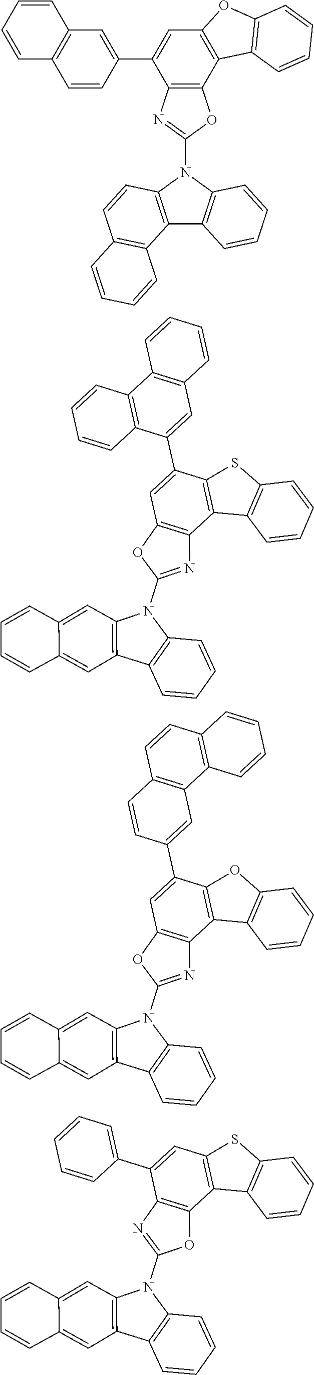

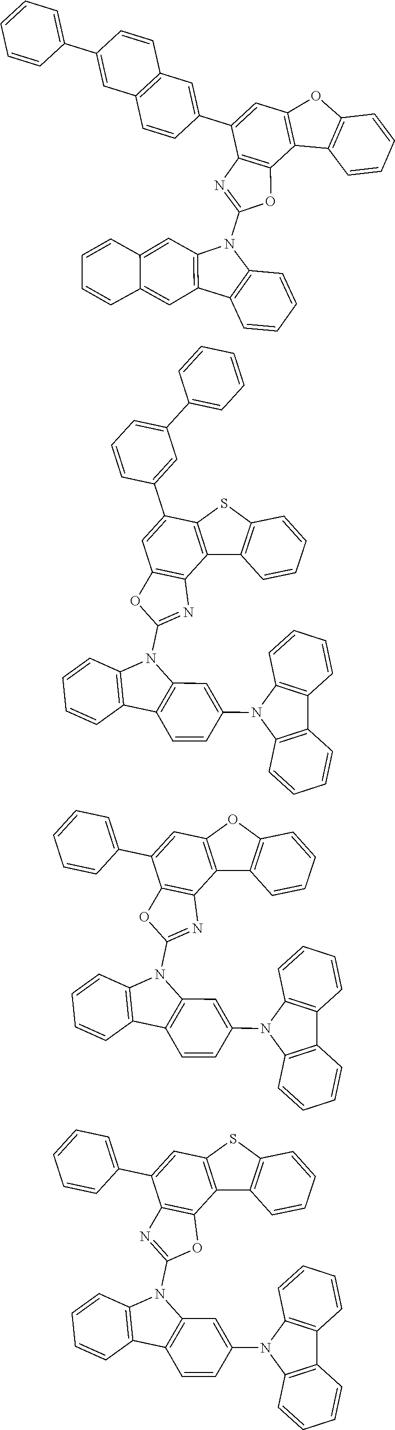

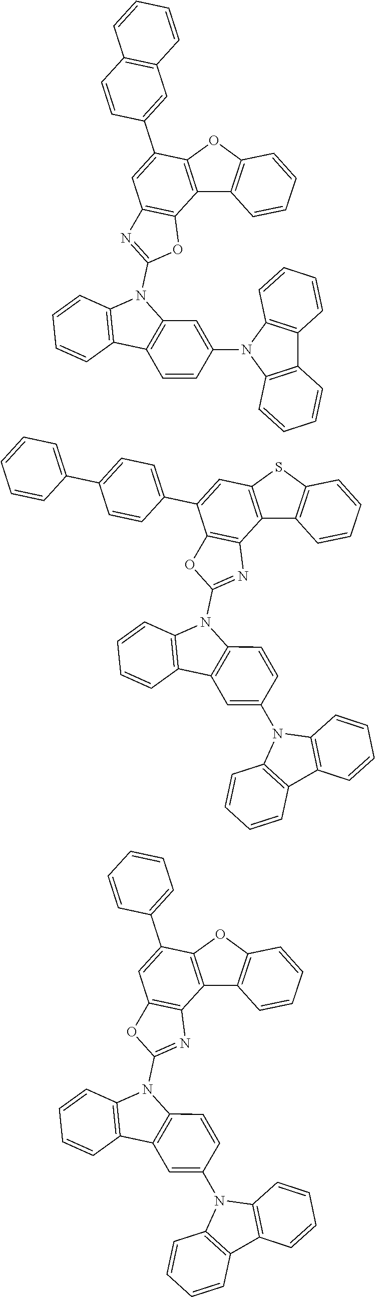

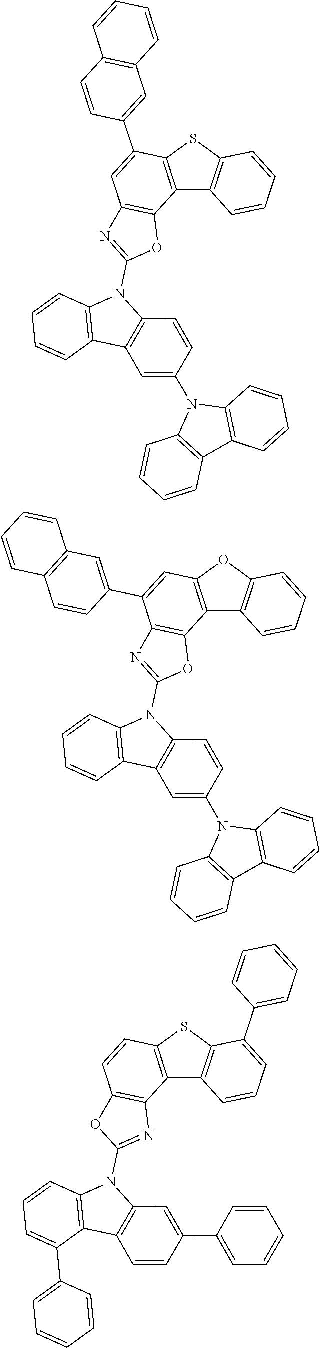

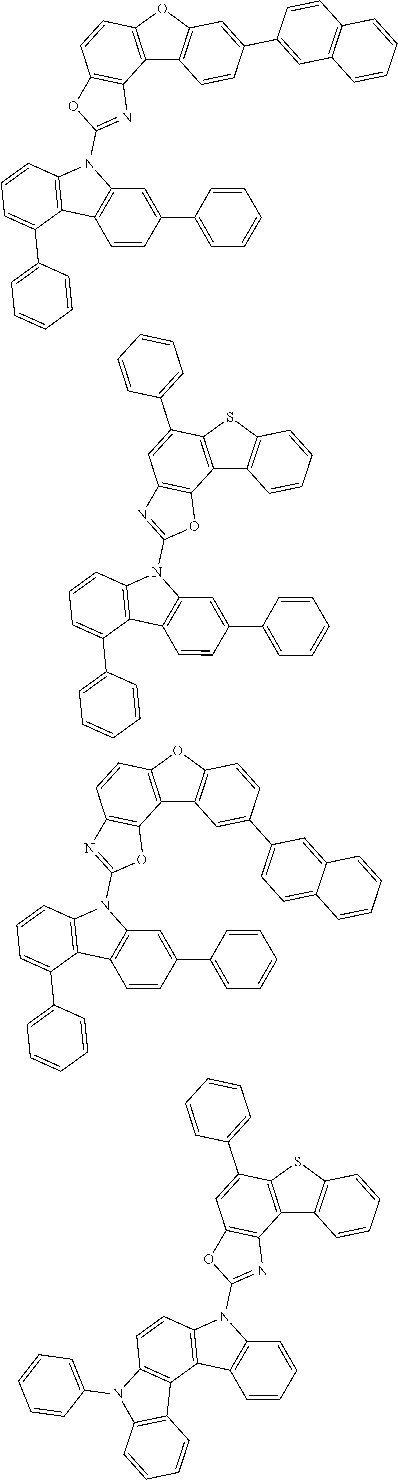

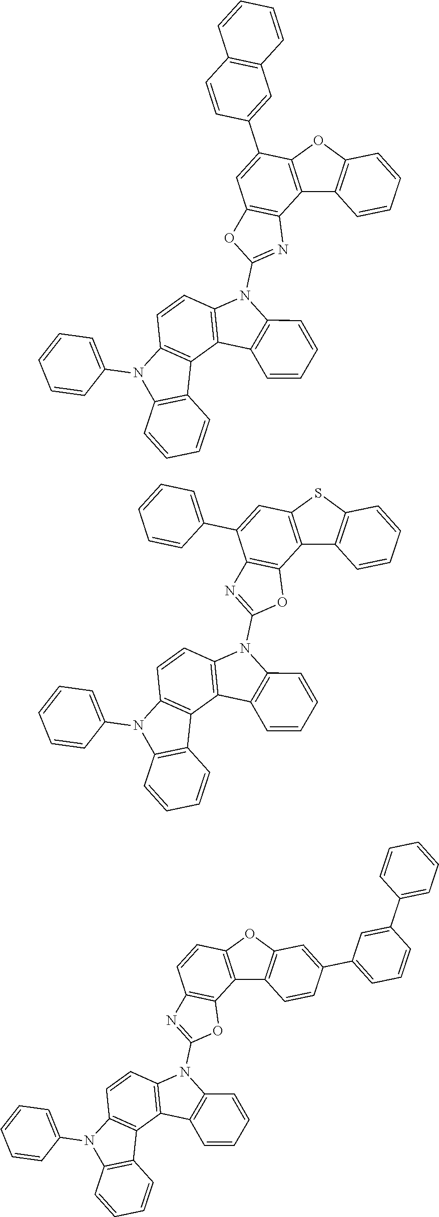

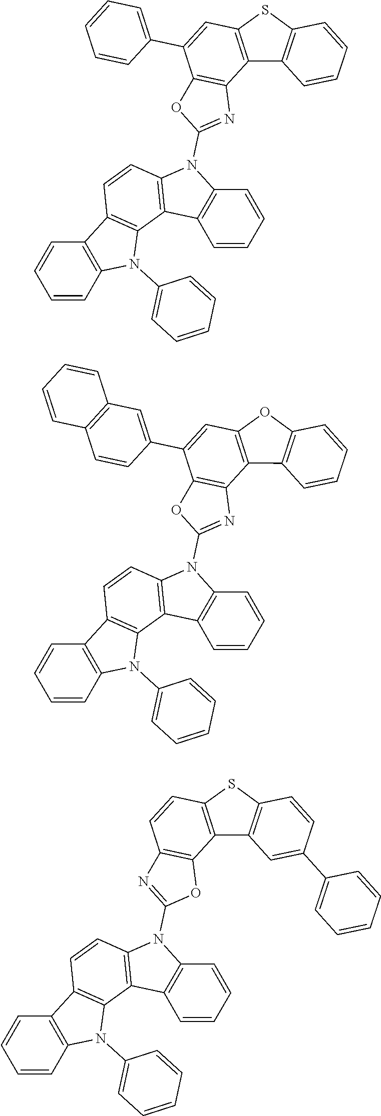 [SMILES: c1ccc(-c2cc3sc4ccccc4c3c3nc(-n4c5ccccc5c5c4ccc4c6ccccc6n(-c6ccccc6)c45)oc23)cc1 ; c1ccc(-n2c3ccccc3c3ccc4c(c5ccccc5n4-c4nc5c(o4)c(-c4ccc6ccccc6c4)cc4oc6ccccc6c45)c32)cc1 ; c1ccc(-c2ccc3sc4ccc5nc(-n6c7ccccc7c7c6ccc6c8ccccc8n(-c8ccccc8)c67)oc5c4c3c2)cc1]

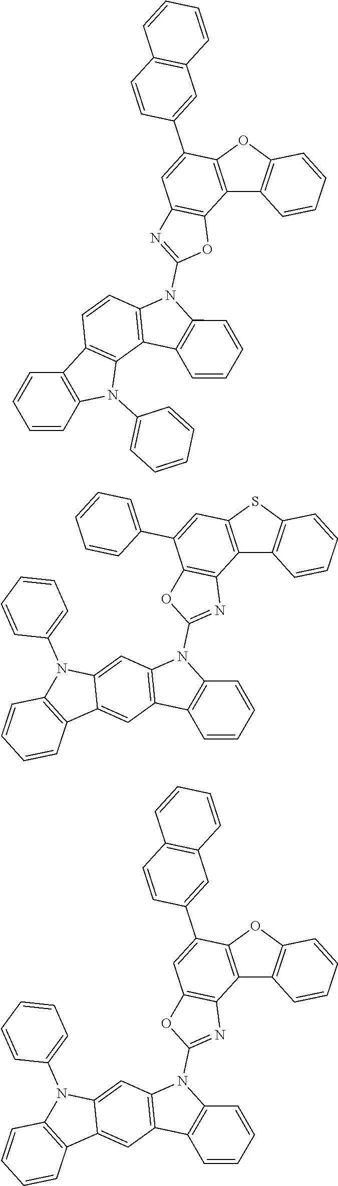

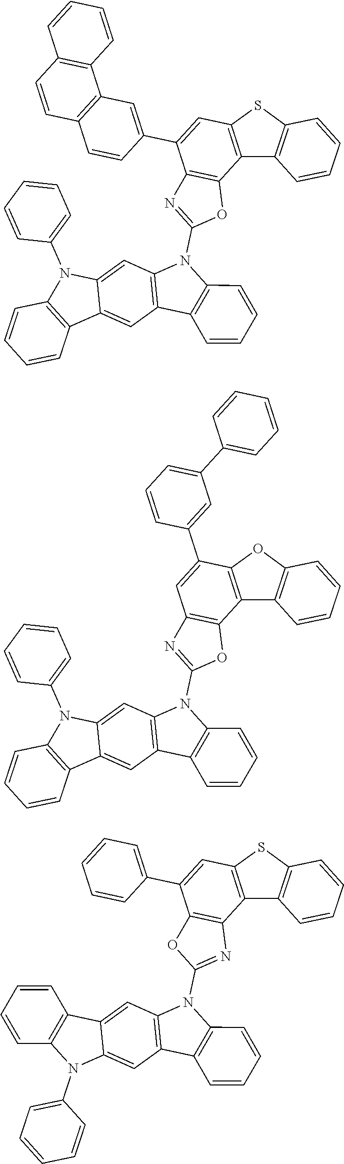 [SMILES: c1ccc(-n2c3ccccc3c3cc4c5ccccc5n(-c5nc6c(-c7ccc8ccc9ccccc9c8c7)cc7sc8ccccc8c7c6o5)c4cc32)cc1 ; c1ccc(-c2cccc(-c3cc4nc(-n5c6ccccc6c6cc7c8ccccc8n(-c8ccccc8)c7cc65)oc4c4c3oc3ccccc34)c2)cc1 ; c1ccc(-c2cc3sc4ccccc4c3c3nc(-n4c5ccccc5c5cc6c(cc54)c4ccccc4n6-c4ccccc4)oc23)cc1]

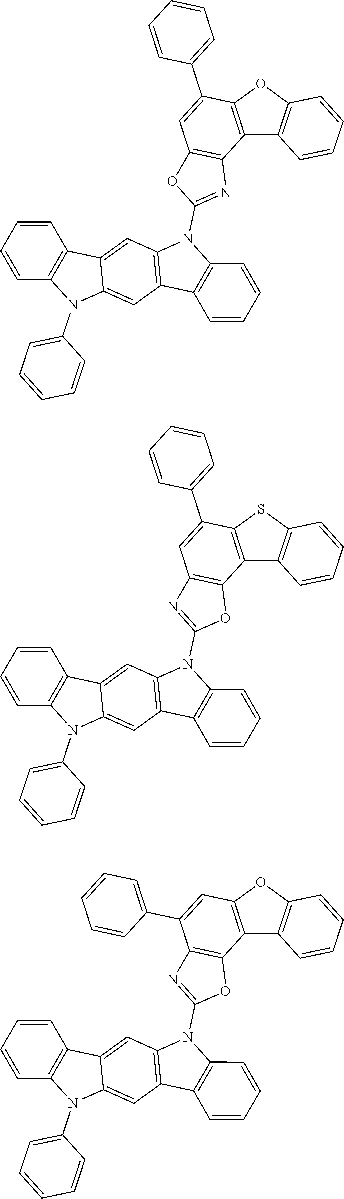 [SMILES: c1ccc(-c2cc3oc(-n4c5ccccc5c5cc6c(cc54)c4ccccc4n6-c4ccccc4)nc3c3c2oc2ccccc23)cc1 ; c1ccc(-c2cc3nc(-n4c5ccccc5c5cc6c(cc54)c4ccccc4n6-c4ccccc4)oc3c3c2sc2ccccc23)cc1 ; c1ccc(-c2cc3oc4ccccc4c3c3oc(-n4c5ccccc5c5cc6c(cc54)c4ccccc4n6-c4ccccc4)nc23)cc1]

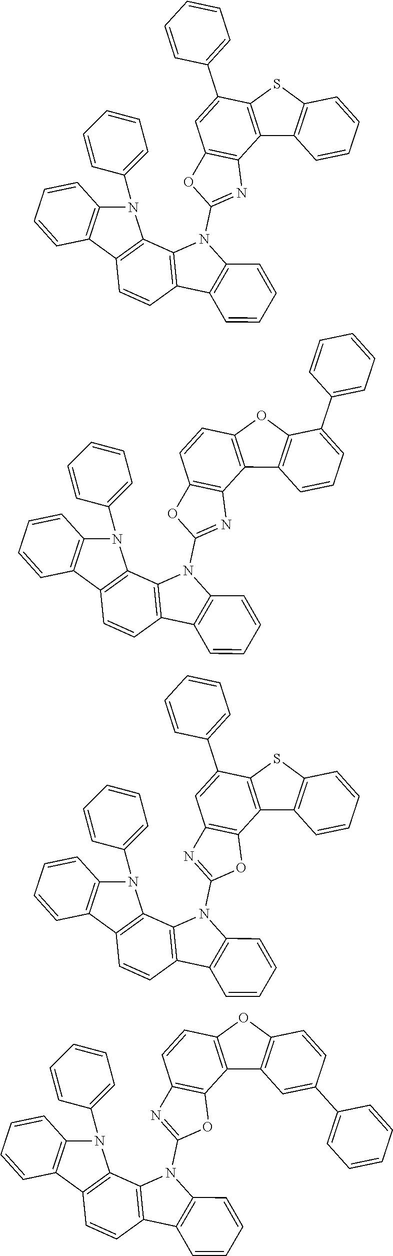

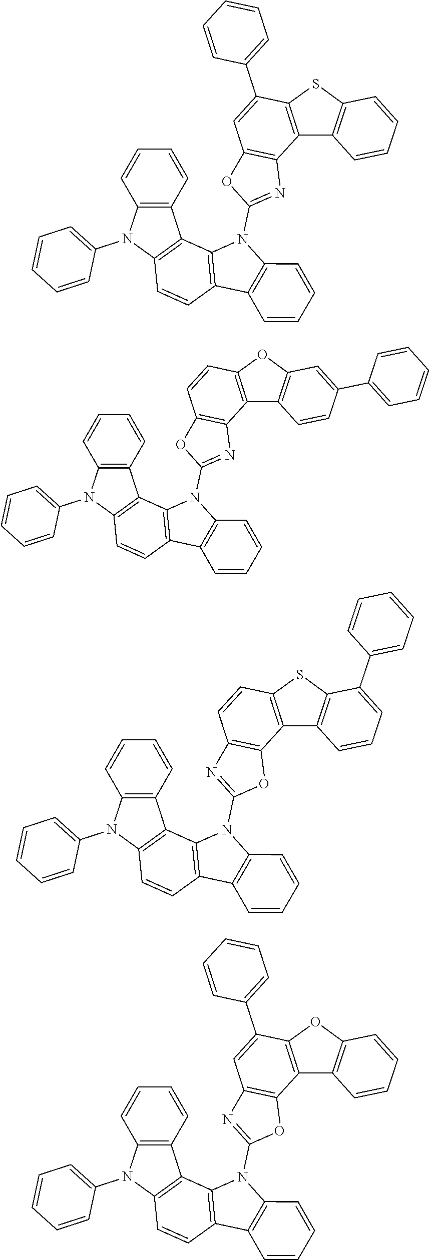

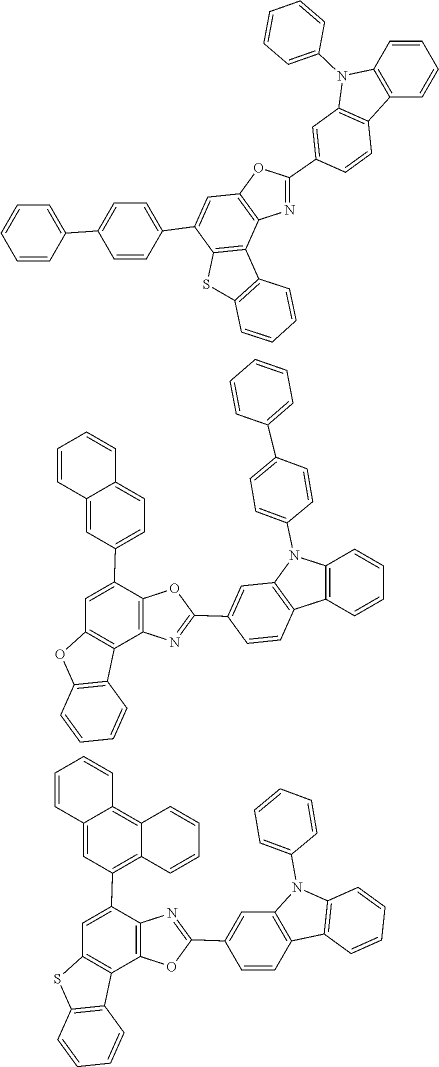

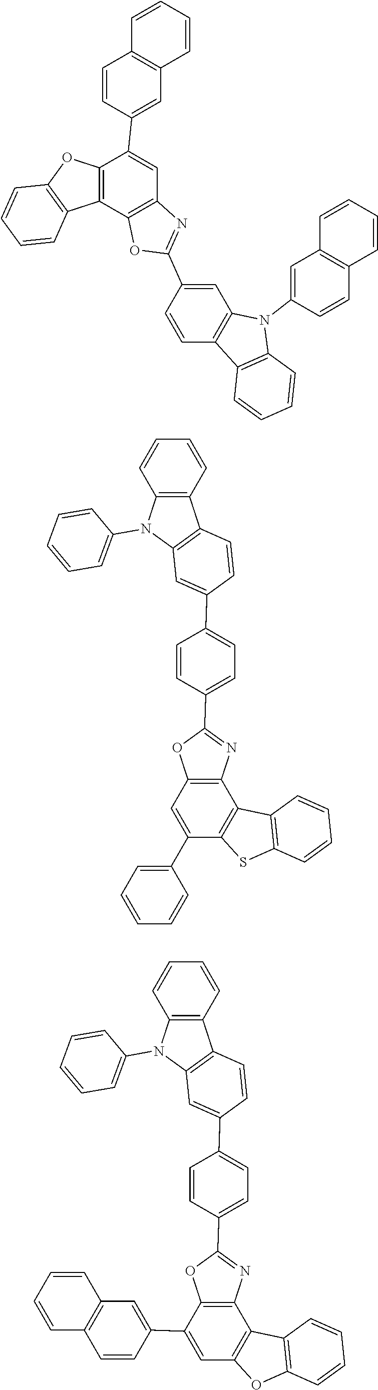

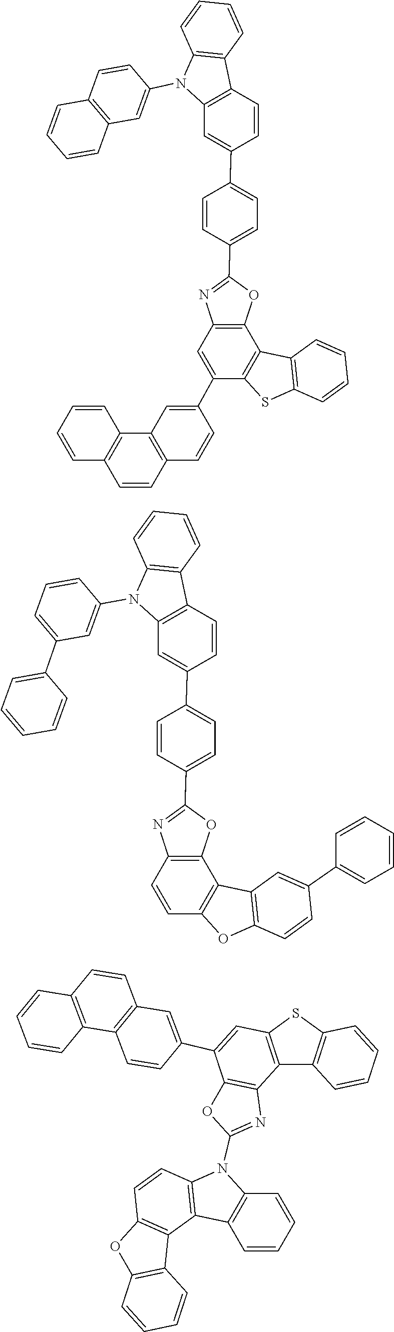

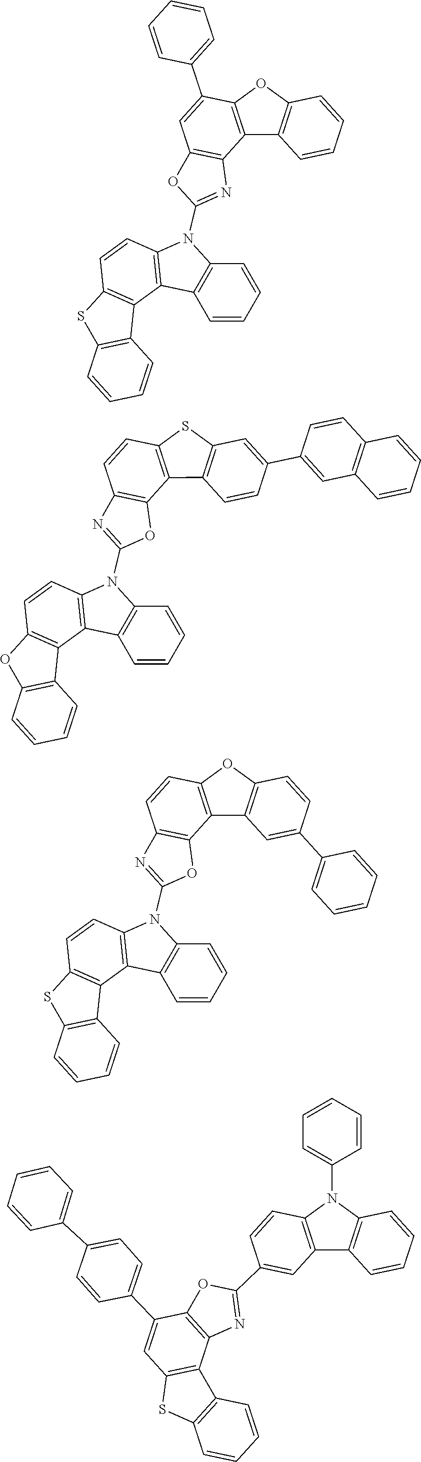

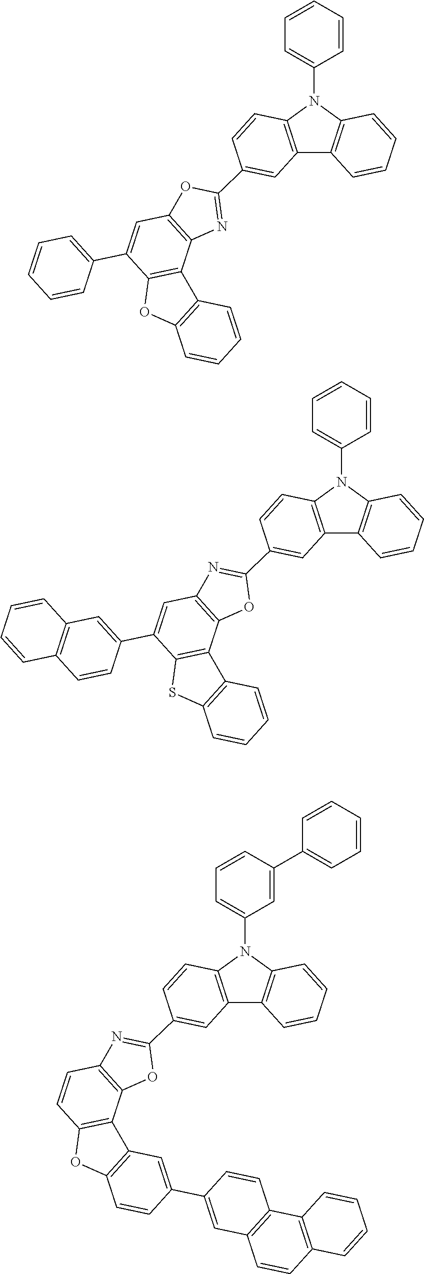 [SMILES: c1ccc(-c2cc3oc(-c4ccc5c(c4)c4ccccc4n5-c4ccccc4)nc3c3c2oc2ccccc23)cc1 ; c1ccc(-n2c3ccccc3c3cc(-c4nc5cc(-c6ccc7ccccc7c6)c6sc7ccccc7c6c5o4)ccc32)cc1 ; c1ccc(-c2cccc(-n3c4ccccc4c4cc(-c5nc6ccc7oc8ccc(-c9ccc%10c(ccc%11ccccc%11%10)c9)cc8c7c6o5)ccc43)c2)cc1]

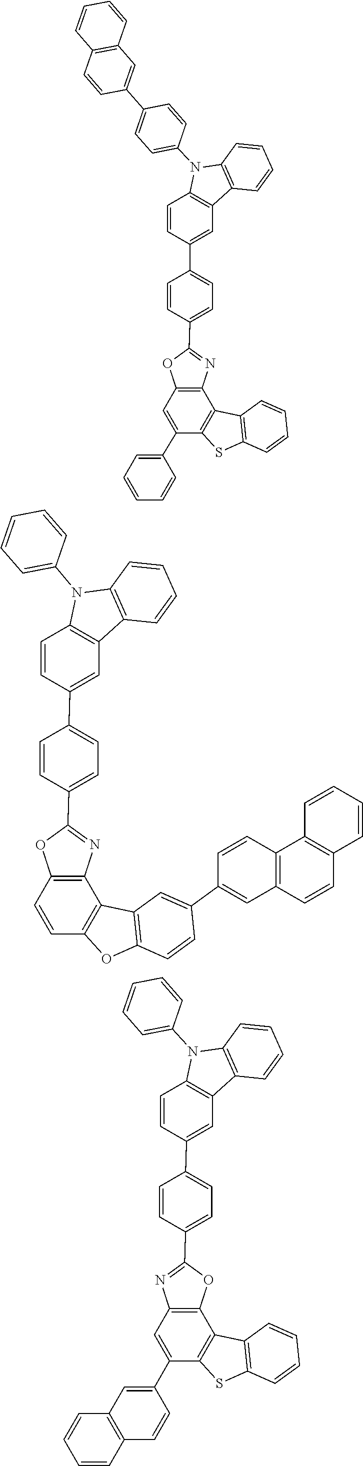

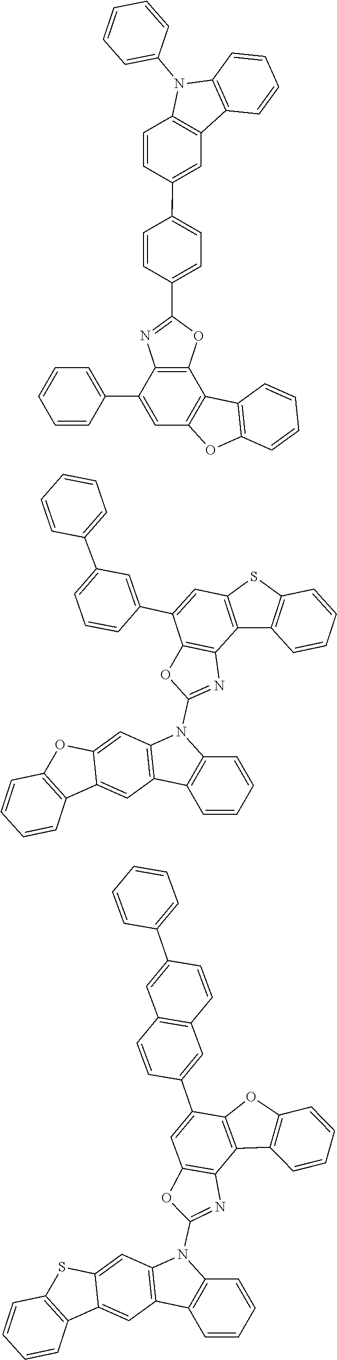 [SMILES: c1ccc(-c2cc3oc4ccccc4c3c3oc(-c4ccc(-c5ccc6c(c5)c5ccccc5n6-c5ccccc5)cc4)nc23)cc1 ; c1ccc(-c2cccc(-c3cc4sc5ccccc5c4c4nc(-n5c6ccccc6c6cc7c(cc65)oc5ccccc57)oc34)c2)cc1 ; c1ccc(-c2ccc3cc(-c4cc5oc(-n6c7ccccc7c7cc8c(cc76)sc6ccccc68)nc5c5c4oc4ccccc45)ccc3c2)cc1]

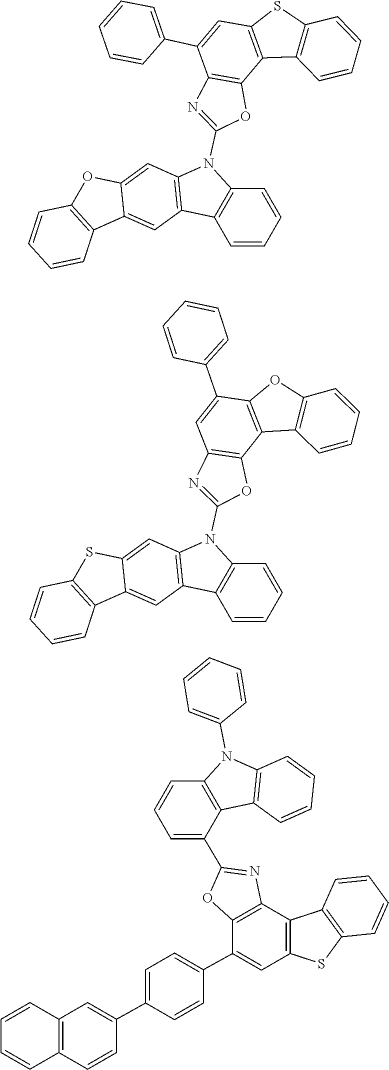

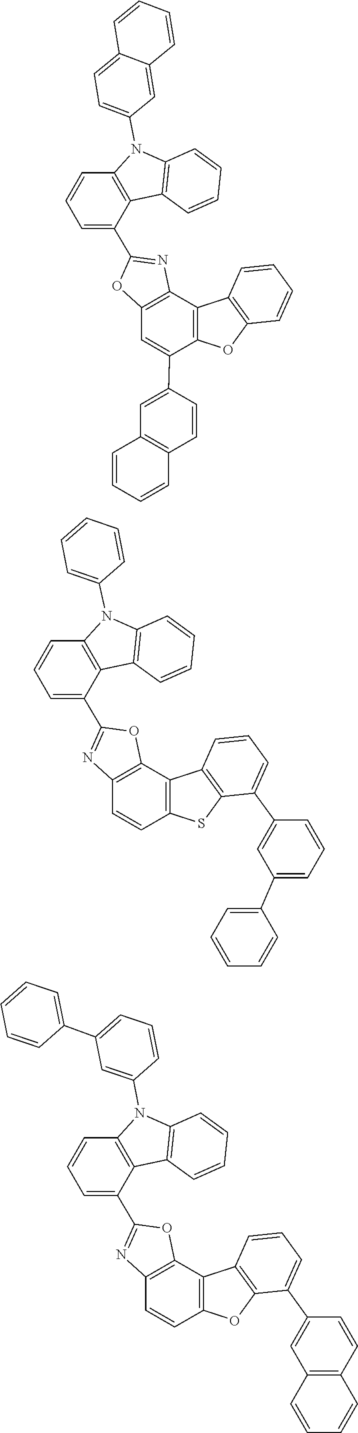

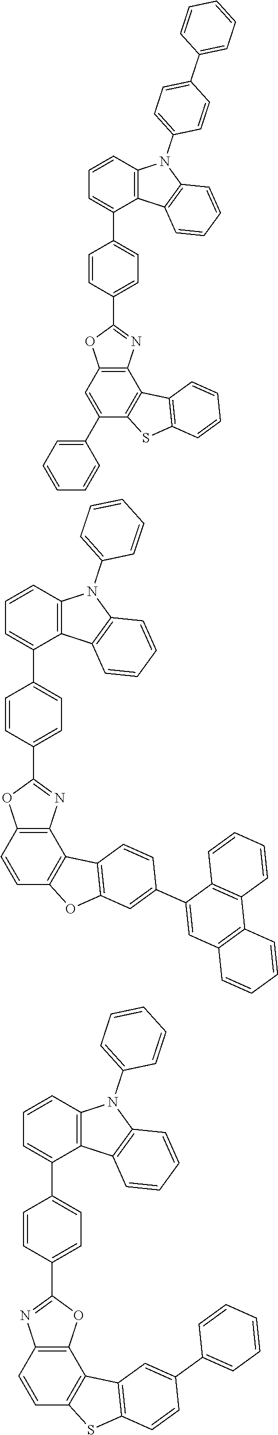

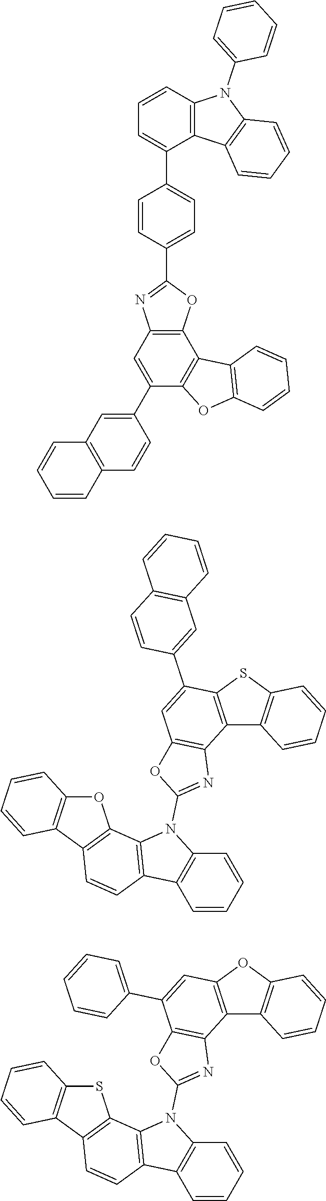 [SMILES: c1ccc(-n2c3ccccc3c3c(-c4ccc(-c5nc6cc(-c7ccc8ccccc8c7)c7oc8ccccc8c7c6o5)cc4)cccc32)cc1 ; c1ccc2cc(-c3cc4oc(-n5c6ccccc6c6ccc7c8ccccc8oc7c65)nc4c4c3sc3ccccc34)ccc2c1 ; c1ccc(-c2cc3oc4ccccc4c3c3nc(-n4c5ccccc5c5ccc6c7ccccc7sc6c54)oc23)cc1]

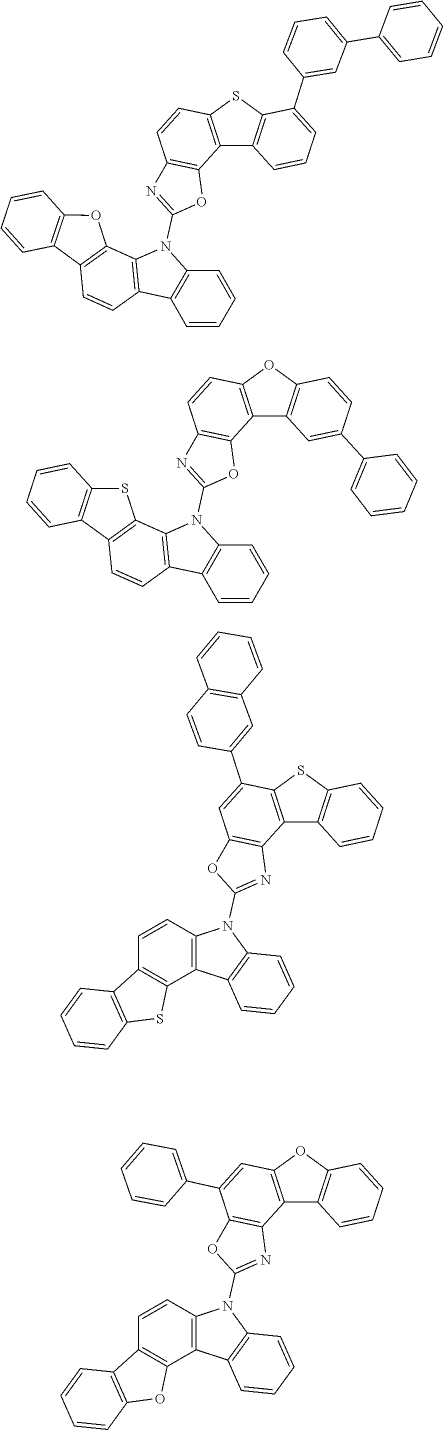

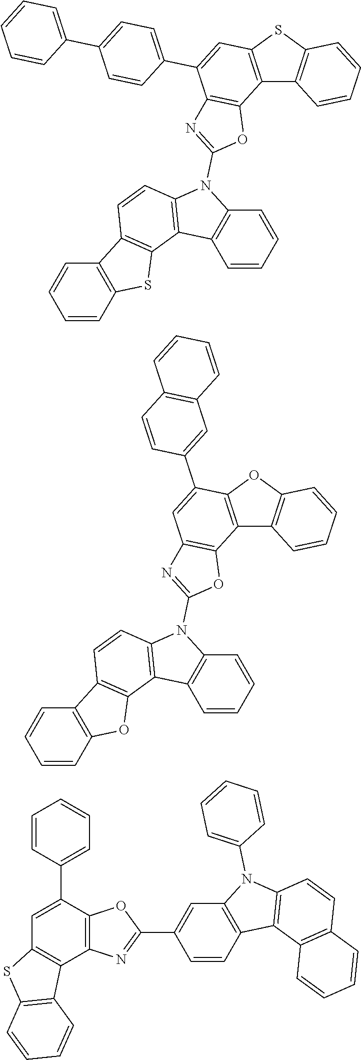

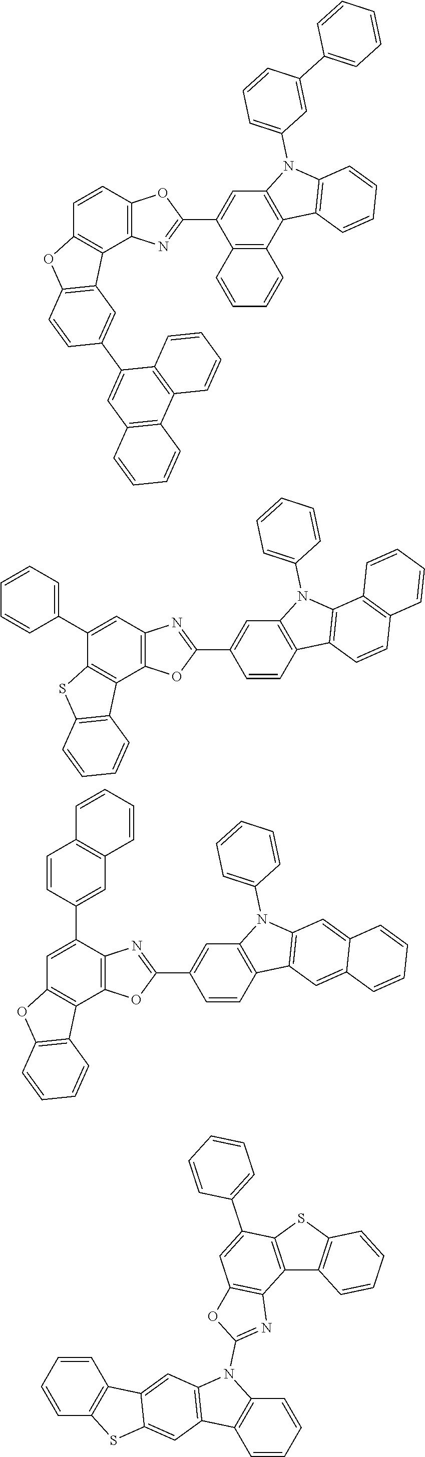

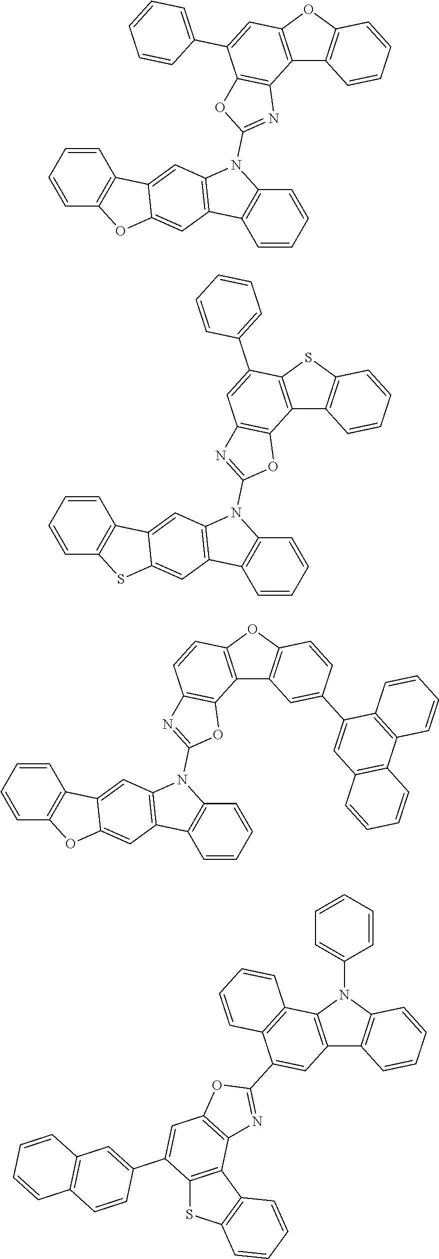

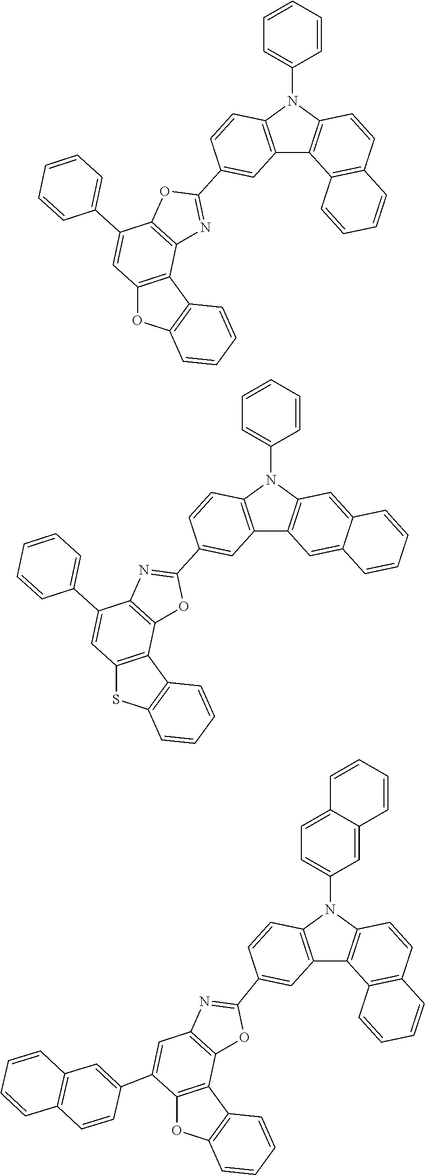

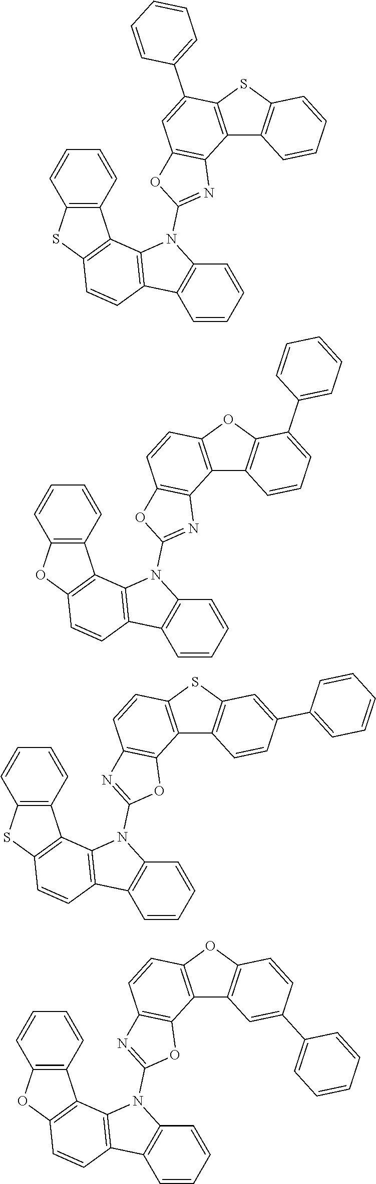

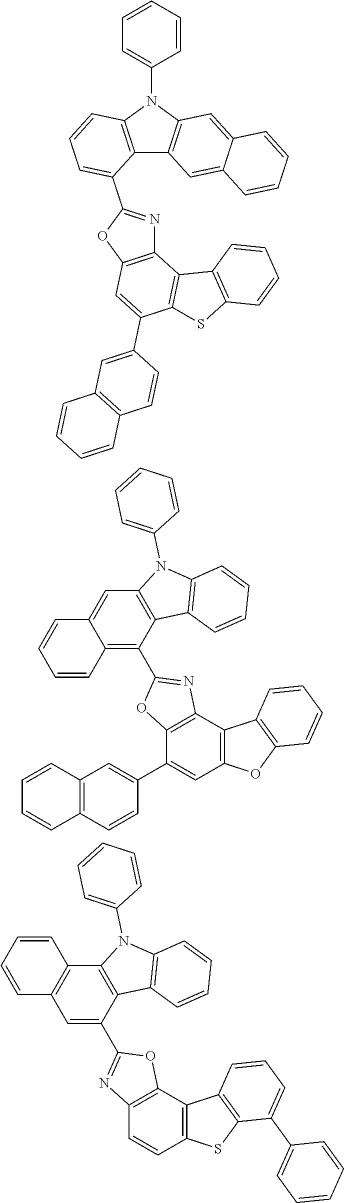

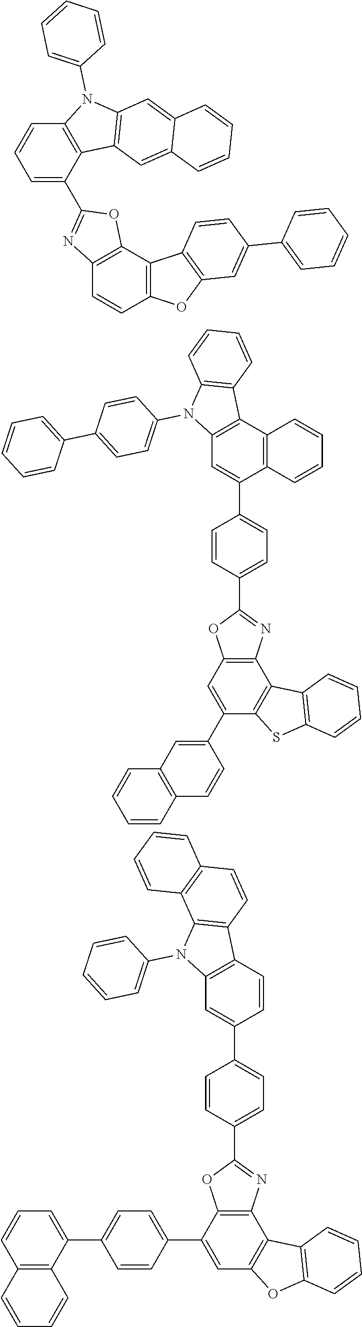

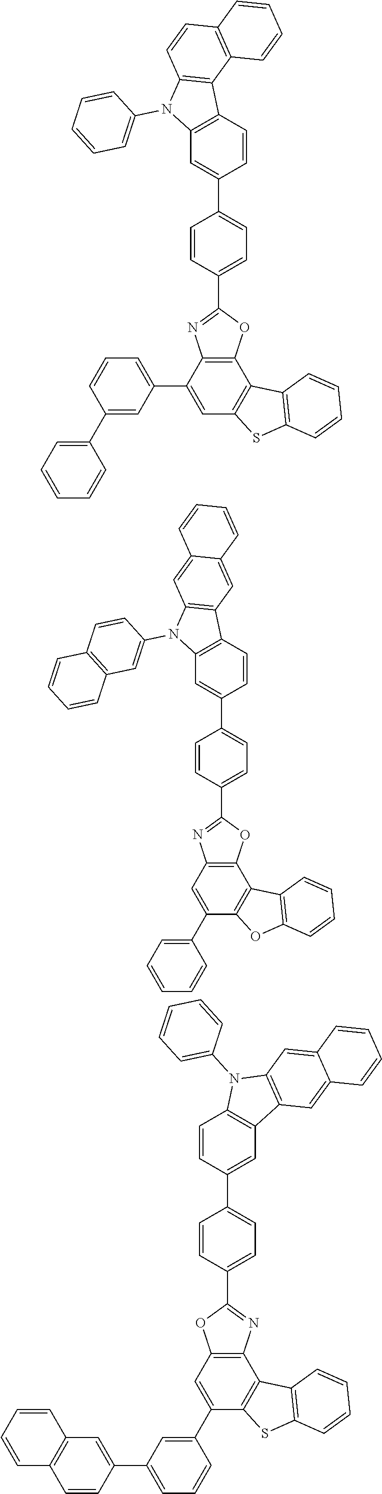

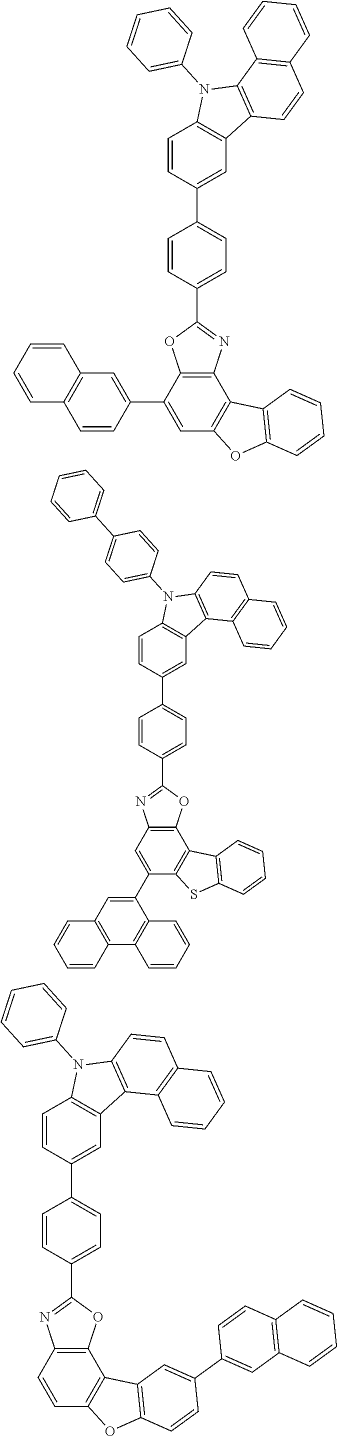

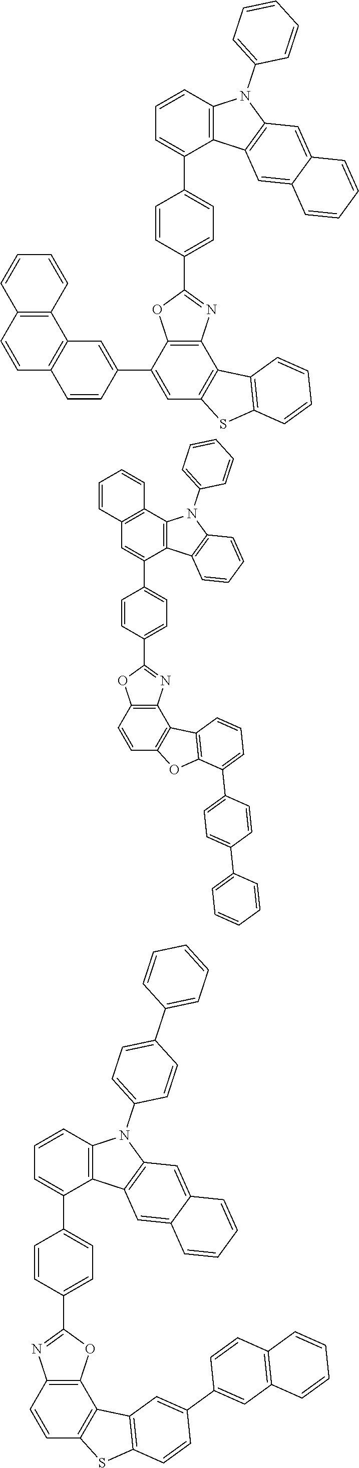

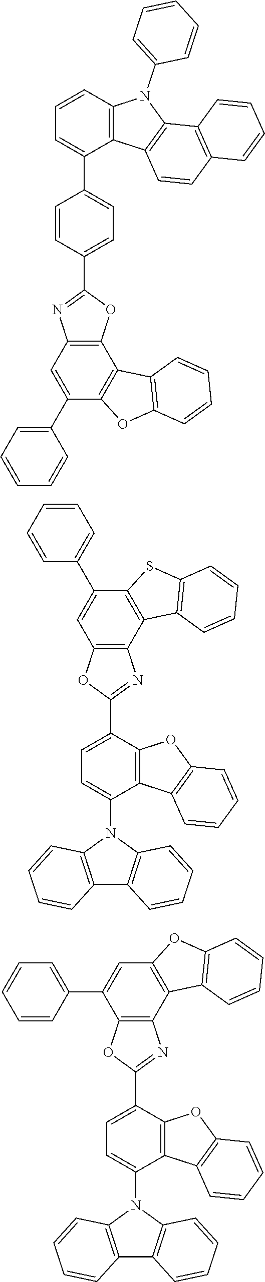

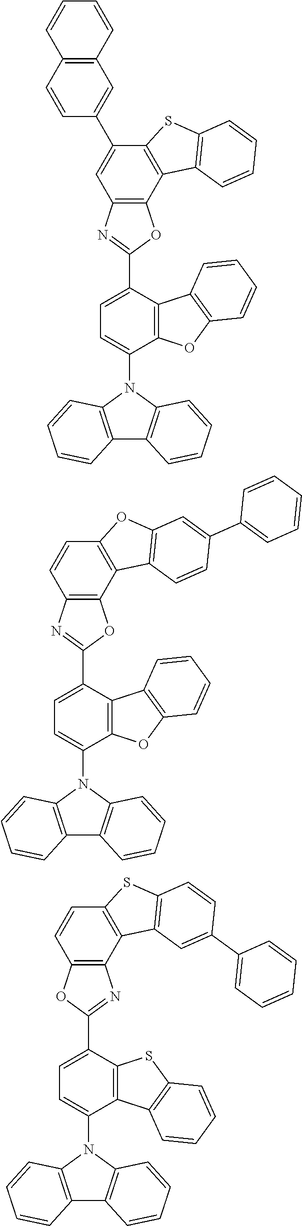

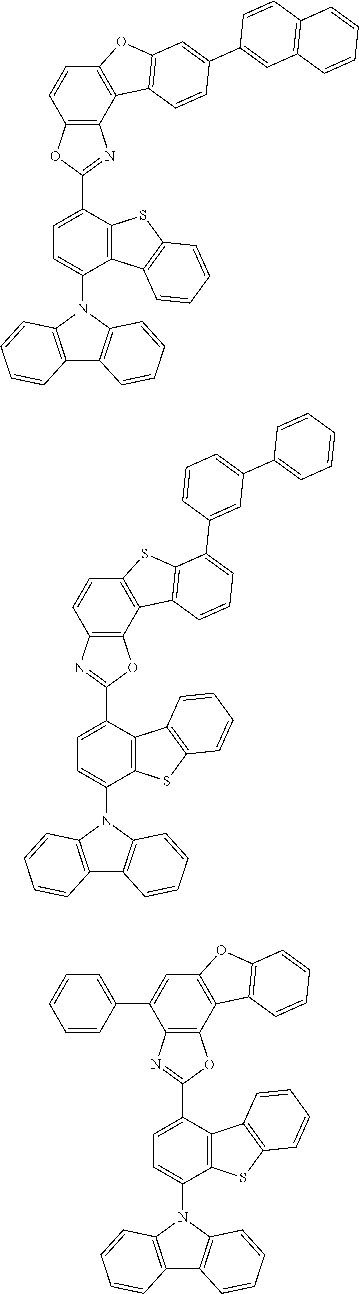 [SMILES: c1ccc2cc(-c3ccc4c(c3)oc3ccc5oc(-c6ccc(-n7c8ccccc8c8ccccc87)c7c6sc6ccccc67)nc5c34)ccc2c1 ; c1ccc(-c2cccc(-c3cccc4c3sc3ccc5nc(-c6ccc(-n7c8ccccc8c8ccccc87)c7sc8ccccc8c67)oc5c34)c2)cc1 ; c1ccc(-c2cc3oc4ccccc4c3c3oc(-c4ccc(-n5c6ccccc6c6ccccc65)c5sc6ccccc6c45)nc23)cc1]

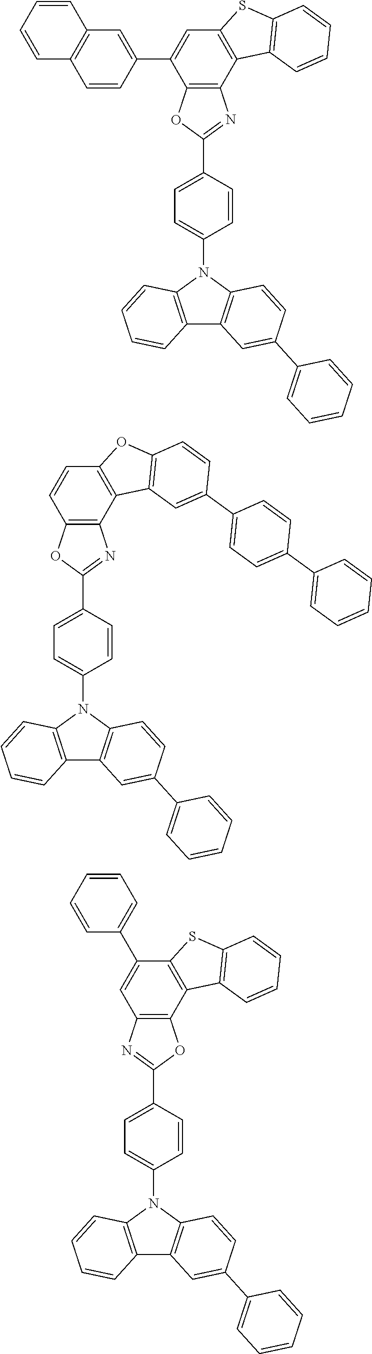 [SMILES: c1ccc(-c2ccc3c(c2)c2ccccc2n3-c2ccc(-c3nc4c(o3)c(-c3ccc5ccccc5c3)cc3sc5ccccc5c34)cc2)cc1 ; c1ccc(-c2ccc(-c3ccc4oc5ccc6oc(-c7ccc(-n8c9ccccc9c9cc(-c%10ccccc%10)ccc98)cc7)nc6c5c4c3)cc2)cc1 ; c1ccc(-c2ccc3c(c2)c2ccccc2n3-c2ccc(-c3nc4cc(-c5ccccc5)c5sc6ccccc6c5c4o3)cc2)cc1]

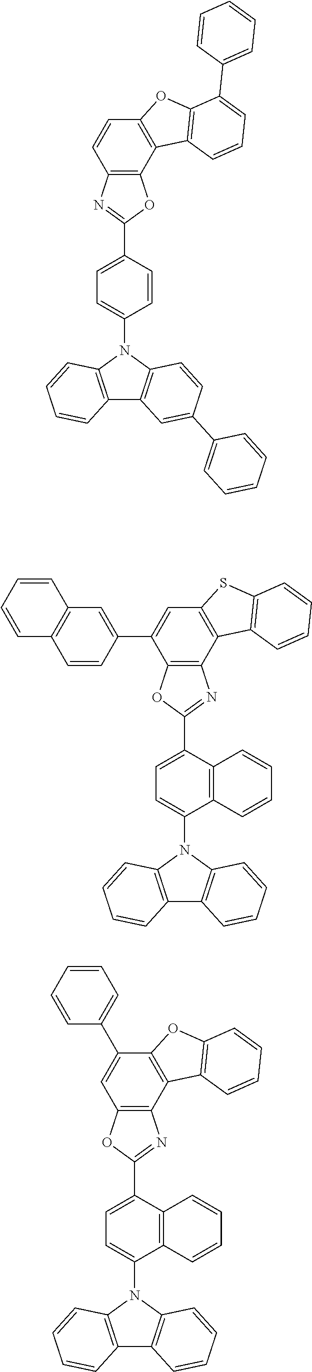 [SMILES: c1ccc(-c2ccc3c(c2)c2ccccc2n3-c2ccc(-c3nc4ccc5oc6c(-c7ccccc7)cccc6c5c4o3)cc2)cc1 ; c1ccc2cc(-c3cc4sc5ccccc5c4c4nc(-c5ccc(-n6c7ccccc7c7ccccc76)c6ccccc56)oc34)ccc2c1 ; c1ccc(-c2cc3oc(-c4ccc(-n5c6ccccc6c6ccccc65)c5ccccc45)nc3c3c2oc2ccccc23)cc1]

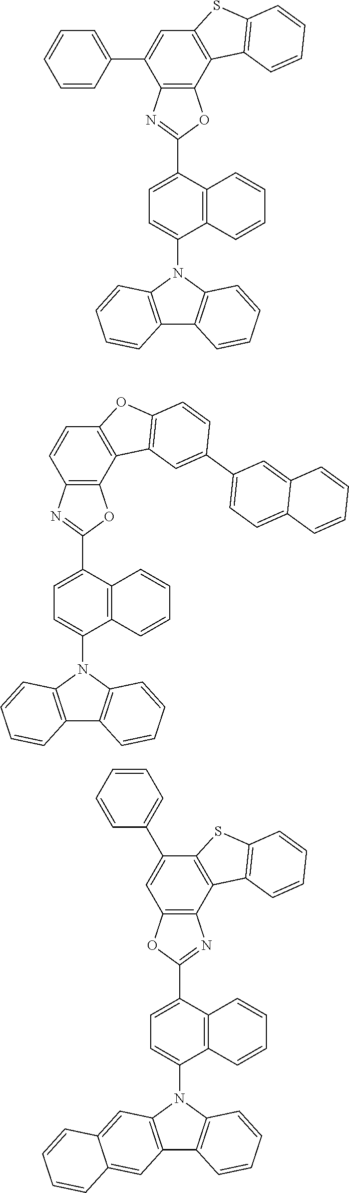

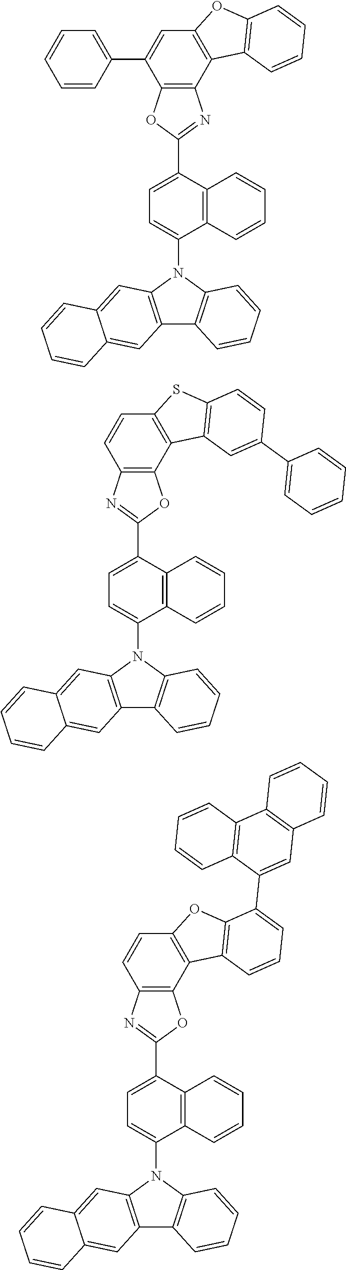

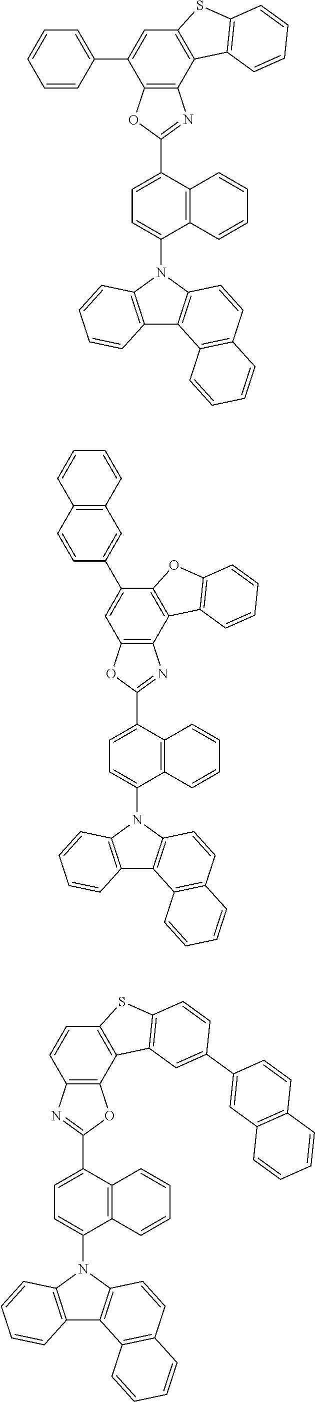 [SMILES: c1ccc(-c2cc3sc4ccccc4c3c3nc(-c4ccc(-n5c6ccccc6c6c7ccccc7ccc65)c5ccccc45)oc23)cc1 ; c1ccc2cc(-c3cc4oc(-c5ccc(-n6c7ccccc7c7c8ccccc8ccc76)c6ccccc56)nc4c4c3oc3ccccc34)ccc2c1 ; c1ccc2cc(-c3ccc4sc5ccc6nc(-c7ccc(-n8c9ccccc9c9c%10ccccc%10ccc98)c8ccccc78)oc6c5c4c3)ccc2c1]

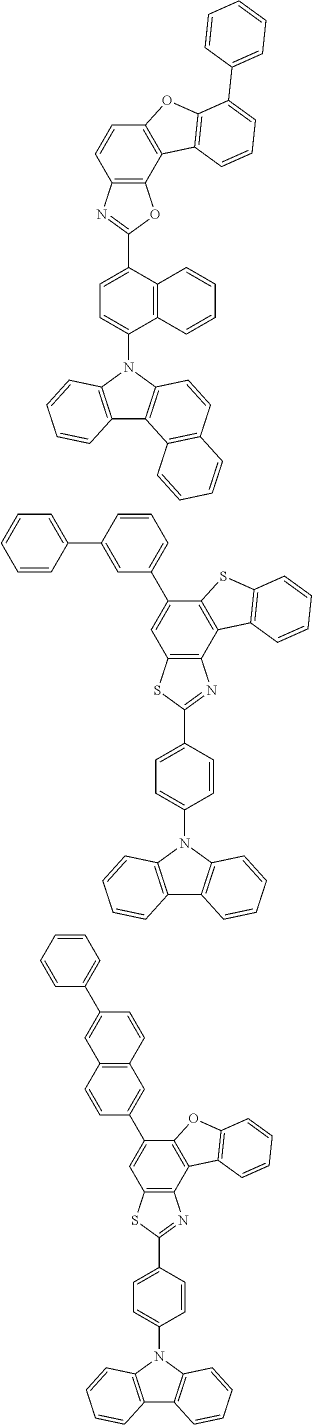

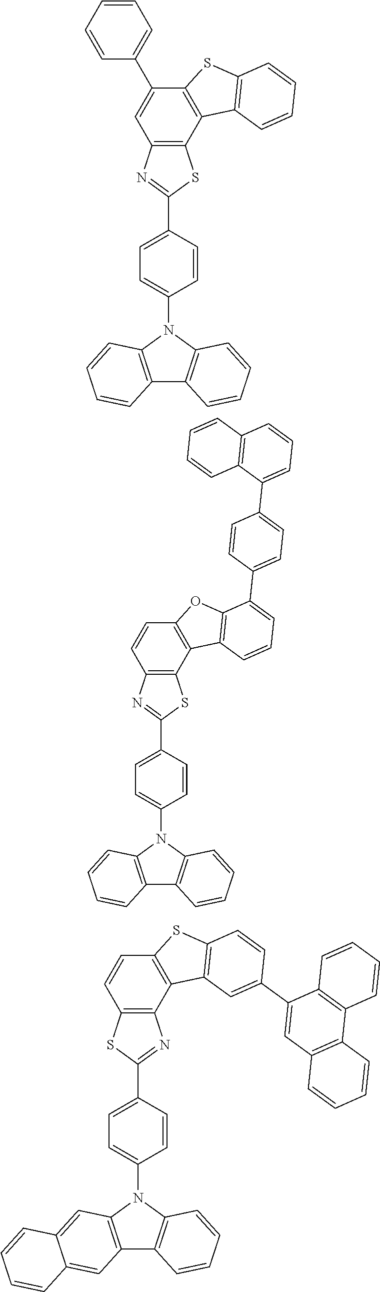

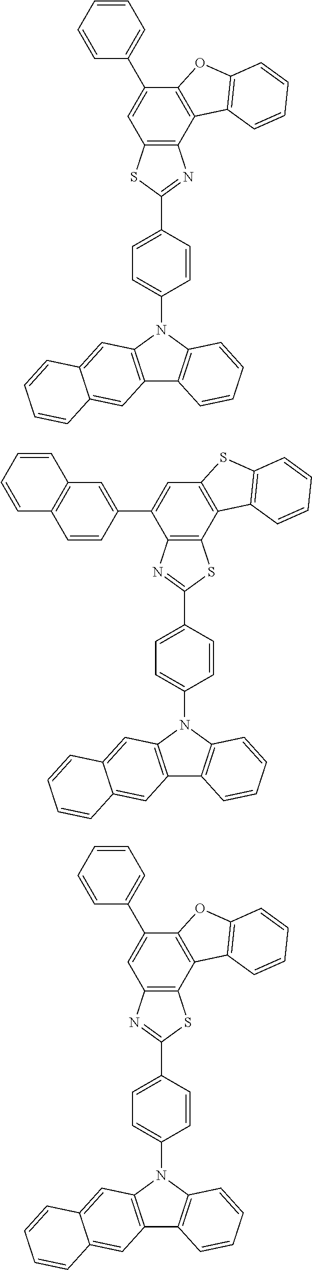

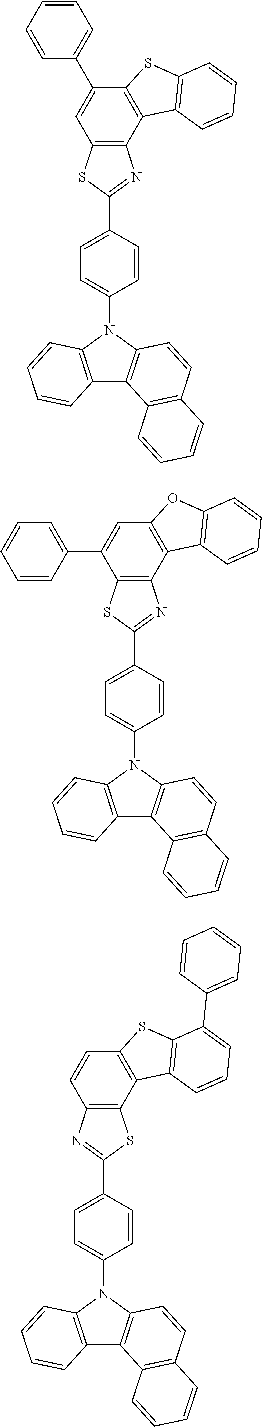

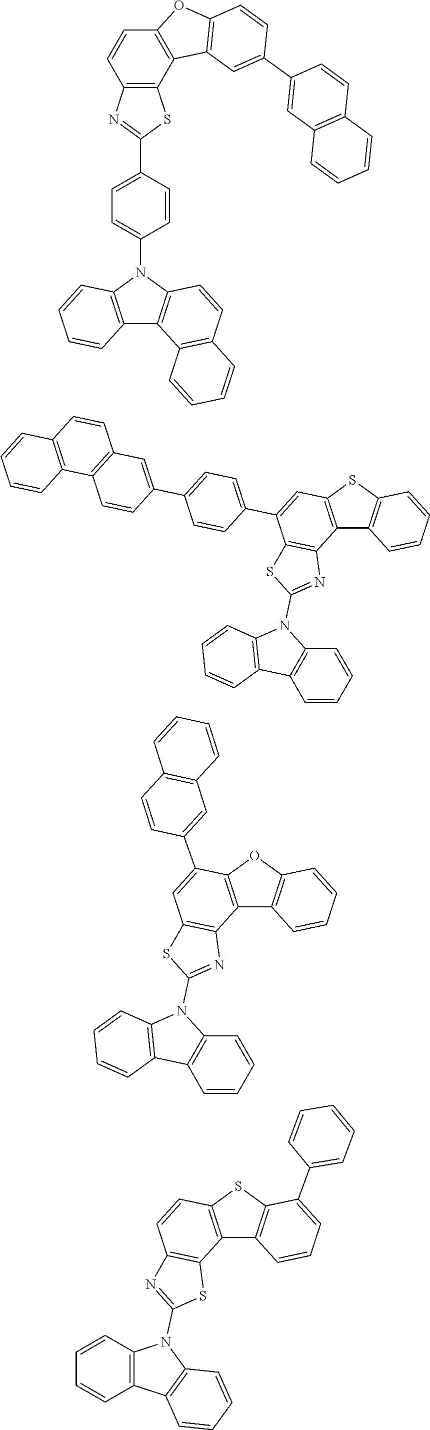

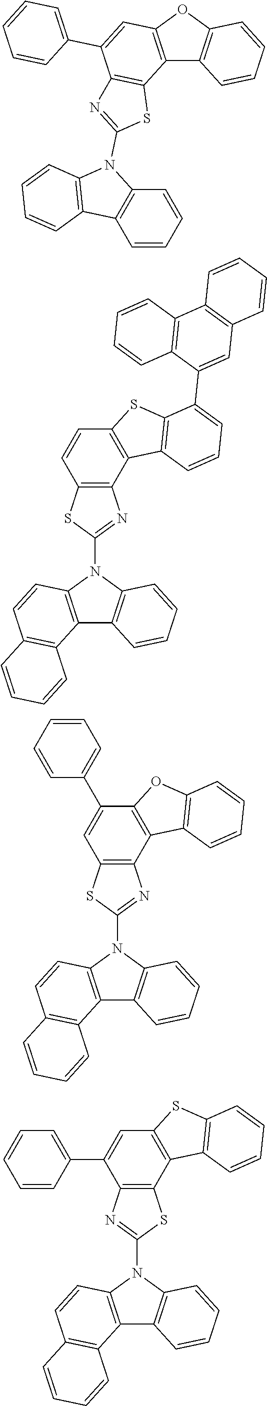

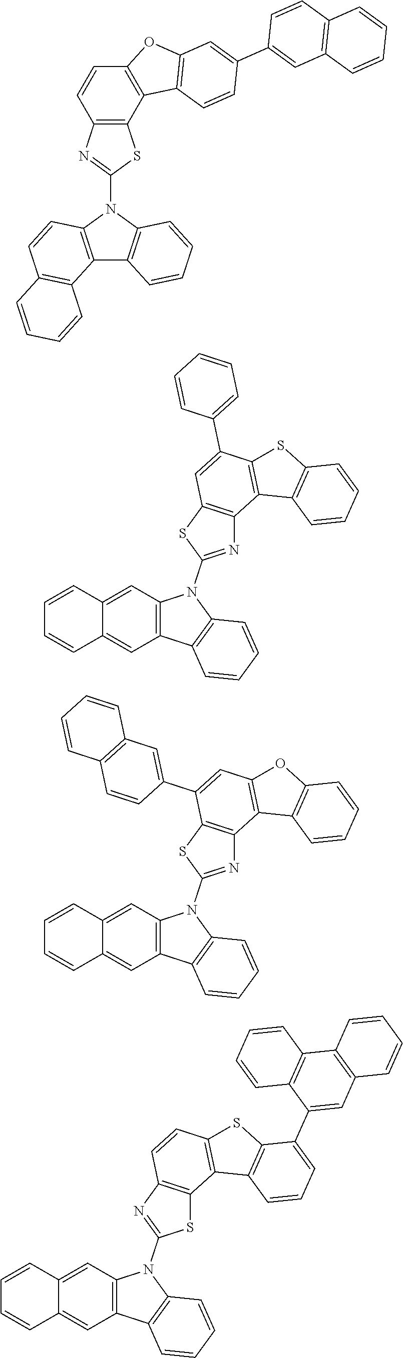

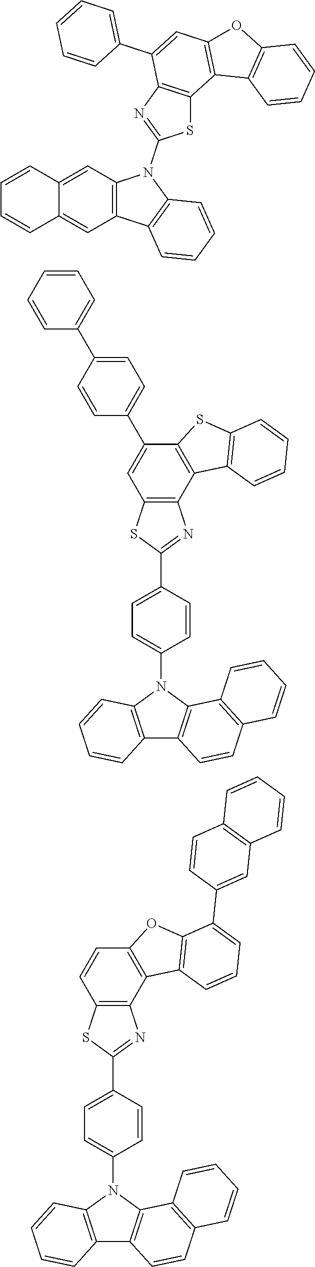

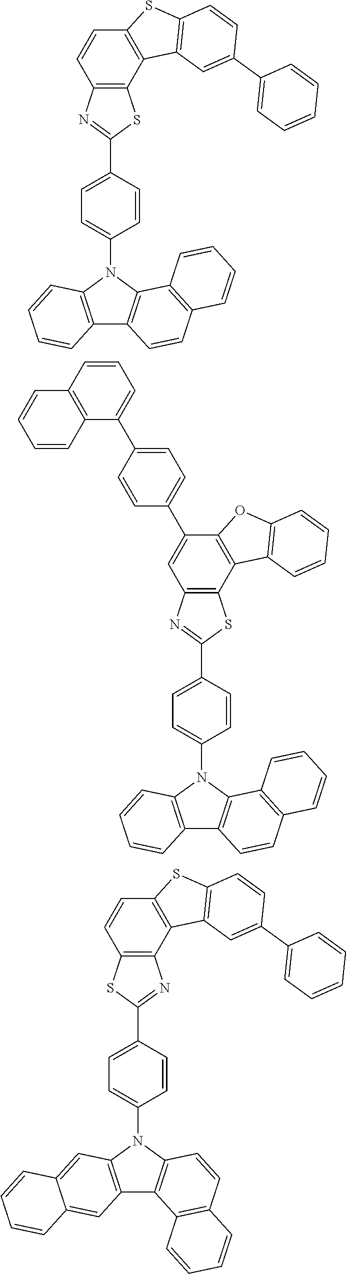

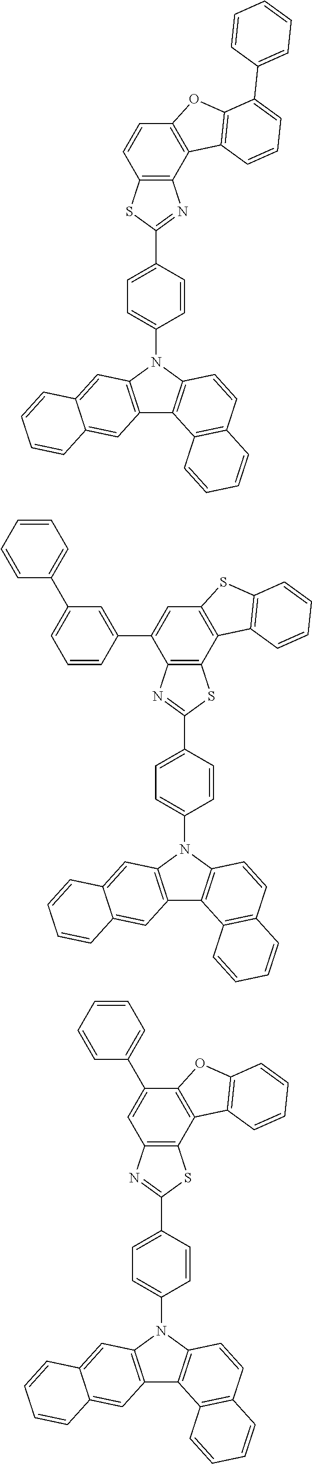 [SMILES: c1ccc(-c2cccc3c2oc2ccc4sc(-c5ccc(-n6c7cc8ccccc8cc7c7c8ccccc8ccc76)cc5)nc4c23)cc1 ; c1ccc(-c2cccc(-c3cc4sc5ccccc5c4c4sc(-c5ccc(-n6c7cc8ccccc8cc7c7c8ccccc8ccc76)cc5)nc34)c2)cc1 ; c1ccc(-c2cc3nc(-c4ccc(-n5c6cc7ccccc7cc6c6c7ccccc7ccc65)cc4)sc3c3c2oc2ccccc23)cc1]

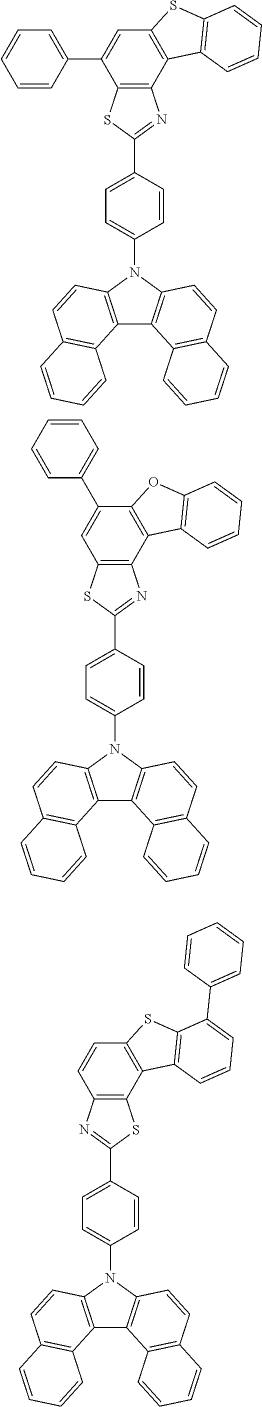

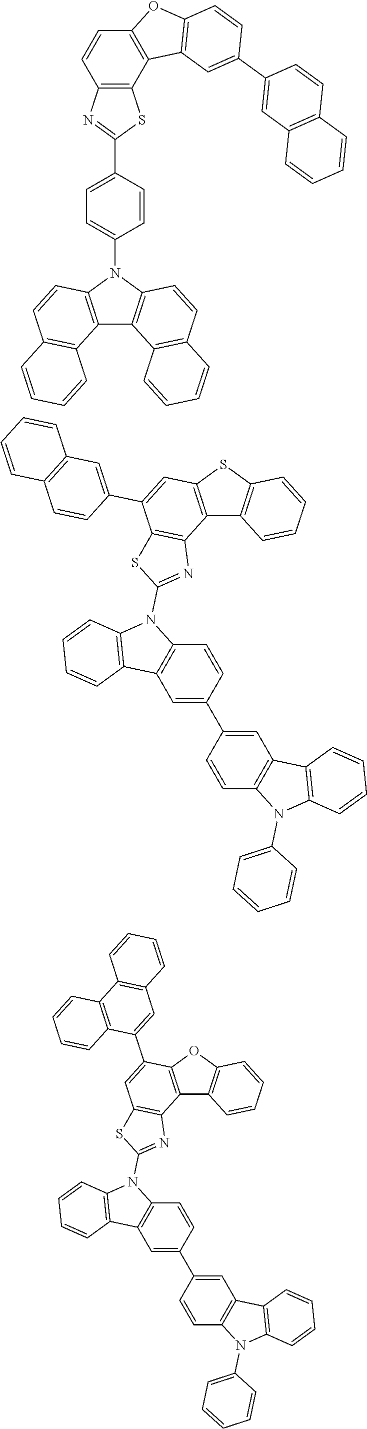

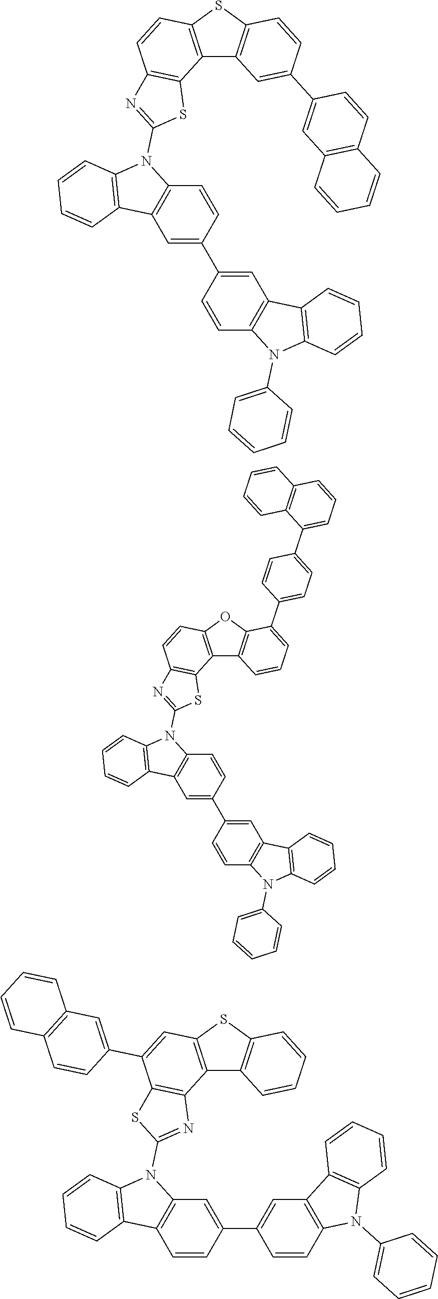

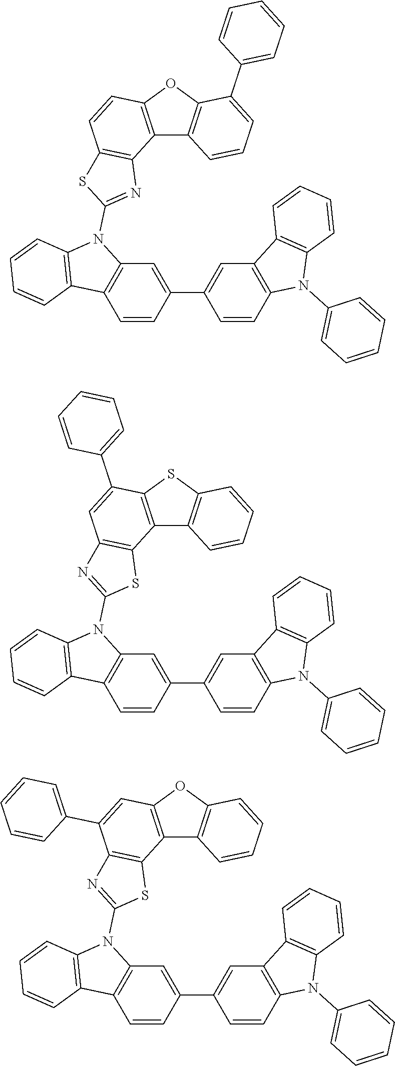 [SMILES: c1ccc(-c2cccc3c2oc2ccc4sc(-n5c6ccccc6c6ccc(-c7ccc8c(c7)c7ccccc7n8-c7ccccc7)cc65)nc4c23)cc1 ; c1ccc(-c2cc3nc(-n4c5ccccc5c5ccc(-c6ccc7c(c6)c6ccccc6n7-c6ccccc6)cc54)sc3c3c2sc2ccccc23)cc1 ; c1ccc(-c2cc3oc4ccccc4c3c3sc(-n4c5ccccc5c5ccc(-c6ccc7c(c6)c6ccccc6n7-c6ccccc6)cc54)nc23)cc1]

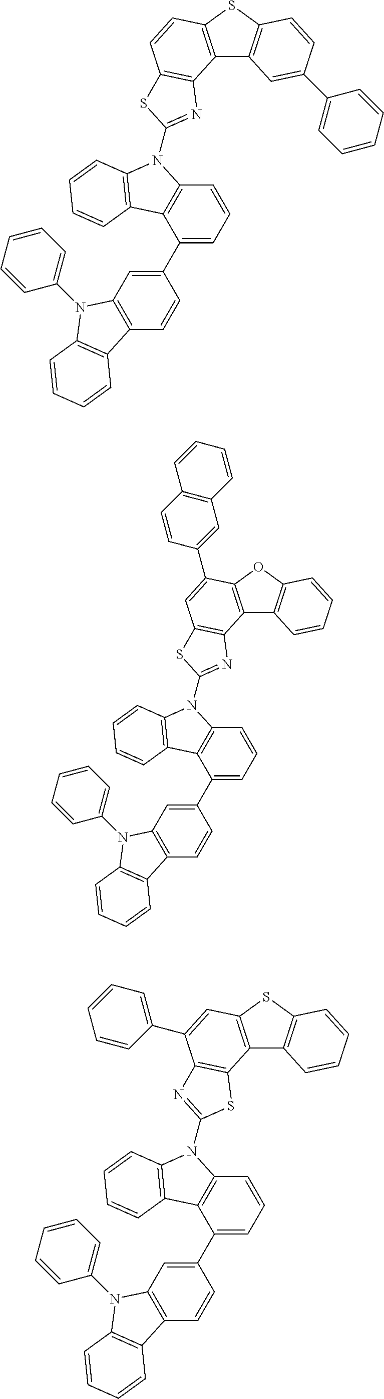 [SMILES: c1ccc(-c2ccc3sc4ccc5sc(-n6c7ccccc7c7c(-c8ccc9c%10ccccc%10n(-c%10ccccc%10)c9c8)cccc76)nc5c4c3c2)cc1 ; c1ccc(-n2c3ccccc3c3ccc(-c4cccc5c4c4ccccc4n5-c4nc5c(cc(-c6ccc7ccccc7c6)c6oc7ccccc7c65)s4)cc32)cc1 ; c1ccc(-c2cc3sc4ccccc4c3c3sc(-n4c5ccccc5c5c(-c6ccc7c8ccccc8n(-c8ccccc8)c7c6)cccc54)nc23)cc1]

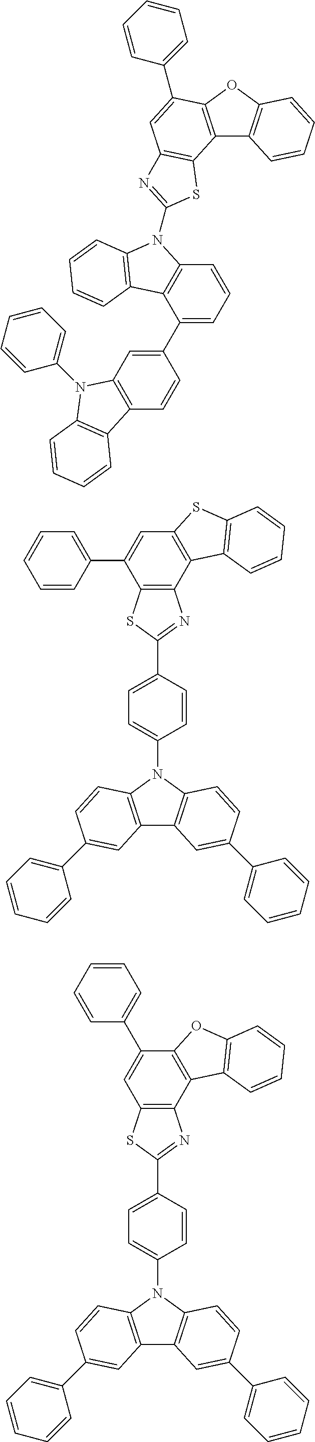

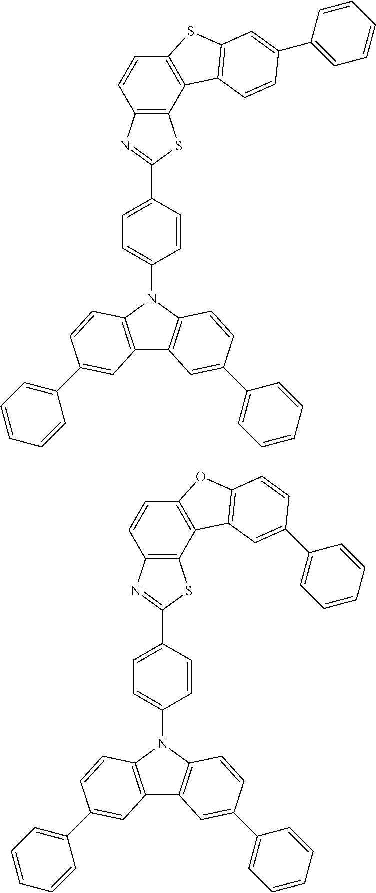

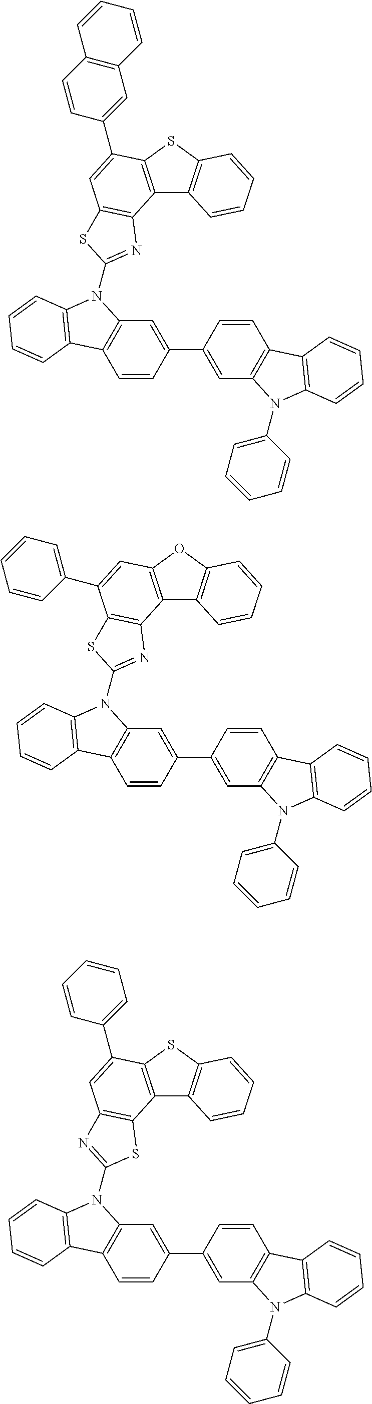 [SMILES: c1ccc(-n2c3ccccc3c3ccc(-c4ccc5c6ccccc6n(-c6nc7c(cc(-c8ccc9ccccc9c8)c8sc9ccccc9c87)s6)c5c4)cc32)cc1 ; c1ccc(-c2cc3oc4ccccc4c3c3nc(-n4c5ccccc5c5ccc(-c6ccc7c8ccccc8n(-c8ccccc8)c7c6)cc54)sc23)cc1 ; c1ccc(-c2cc3nc(-n4c5ccccc5c5ccc(-c6ccc7c8ccccc8n(-c8ccccc8)c7c6)cc54)sc3c3c2sc2ccccc23)cc1]

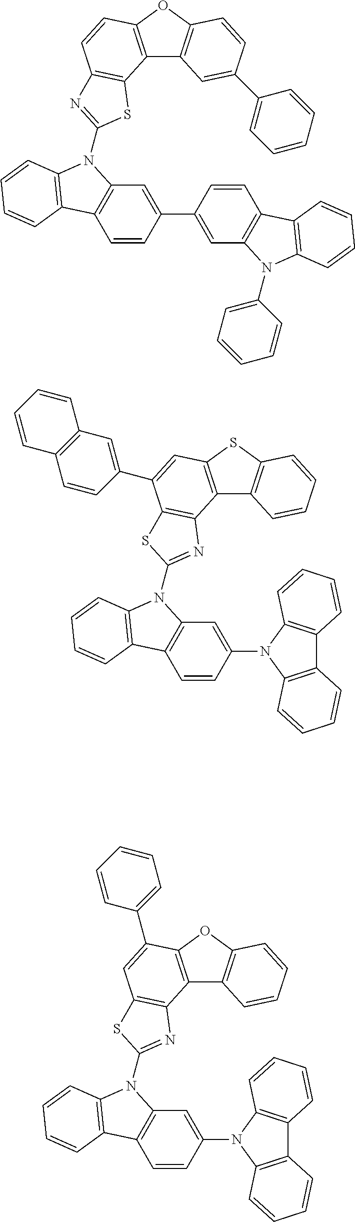 [SMILES: c1ccc(-c2ccc3oc4ccc5nc(-n6c7ccccc7c7ccc(-c8ccc9c%10ccccc%10n(-c%10ccccc%10)c9c8)cc76)sc5c4c3c2)cc1 ; c1ccc2cc(-c3cc4sc5ccccc5c4c4nc(-n5c6ccccc6c6ccc(-n7c8ccccc8c8ccccc87)cc65)sc34)ccc2c1 ; c1ccc(-c2cc3sc(-n4c5ccccc5c5ccc(-n6c7ccccc7c7ccccc76)cc54)nc3c3c2oc2ccccc23)cc1]

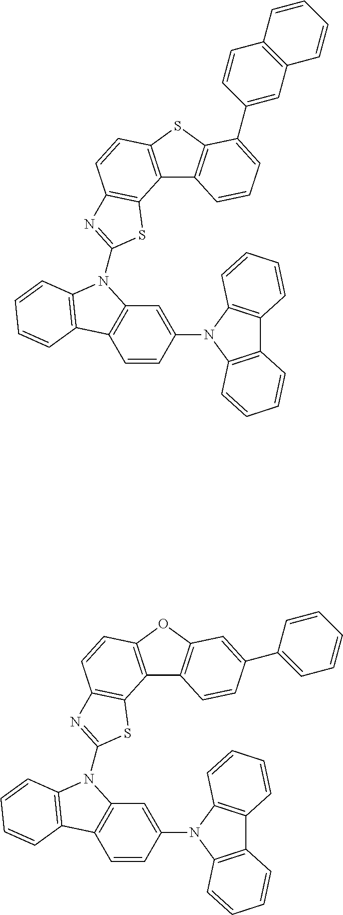 [SMILES: c1ccc2cc(-c3cccc4c3sc3ccc5nc(-n6c7ccccc7c7ccc(-n8c9ccccc9c9ccccc98)cc76)sc5c34)ccc2c1 ; c1ccc(-c2ccc3c(c2)oc2ccc4nc(-n5c6ccccc6c6ccc(-n7c8ccccc8c8ccccc87)cc65)sc4c23)cc1]

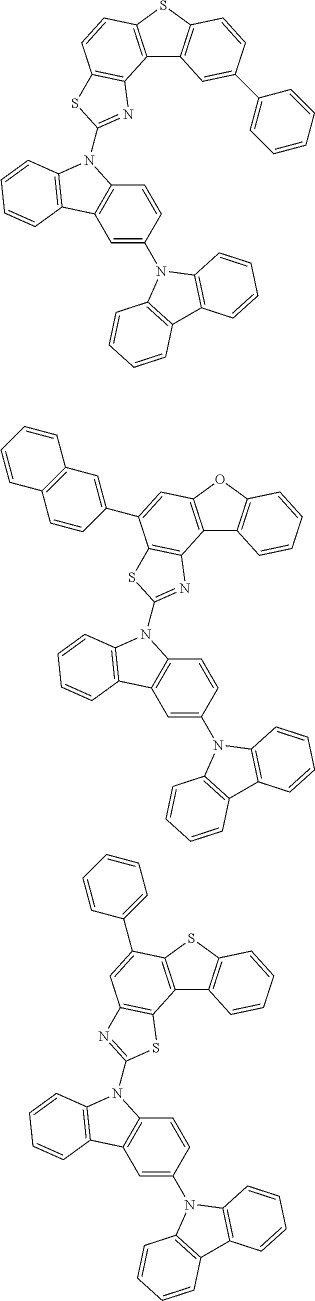 [SMILES: c1ccc(-c2ccc3sc4ccc5sc(-n6c7ccccc7c7cc(-n8c9ccccc9c9ccccc98)ccc76)nc5c4c3c2)cc1 ; c1ccc2cc(-c3cc4oc5ccccc5c4c4nc(-n5c6ccccc6c6cc(-n7c8ccccc8c8ccccc87)ccc65)sc34)ccc2c1 ; c1ccc(-c2cc3nc(-n4c5ccccc5c5cc(-n6c7ccccc7c7ccccc76)ccc54)sc3c3c2sc2ccccc23)cc1]

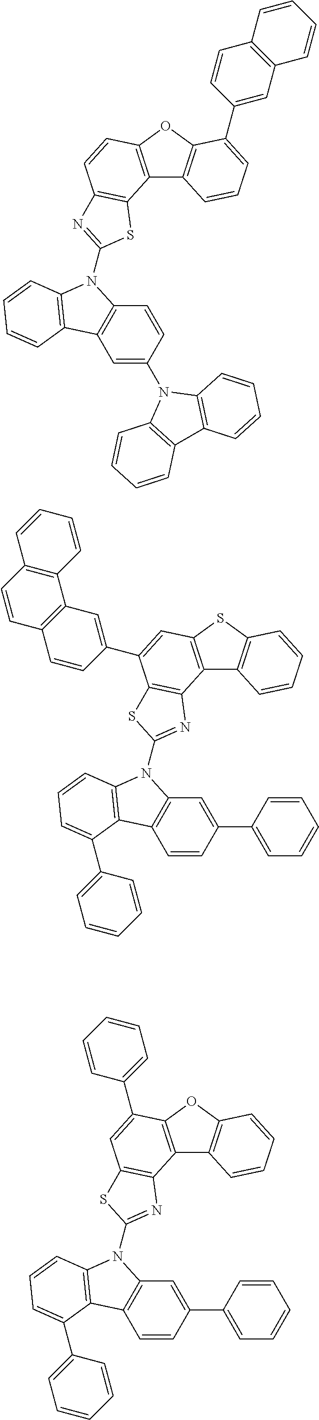 [SMILES: c1ccc2cc(-c3cccc4c3oc3ccc5nc(-n6c7ccccc7c7cc(-n8c9ccccc9c9ccccc98)ccc76)sc5c34)ccc2c1 ; c1ccc(-c2ccc3c4c(-c5ccccc5)cccc4n(-c4nc5c(s4)c(-c4ccc6ccc7ccccc7c6c4)cc4sc6ccccc6c45)c3c2)cc1 ; c1ccc(-c2ccc3c4c(-c5ccccc5)cccc4n(-c4nc5c(cc(-c6ccccc6)c6oc7ccccc7c65)s4)c3c2)cc1]

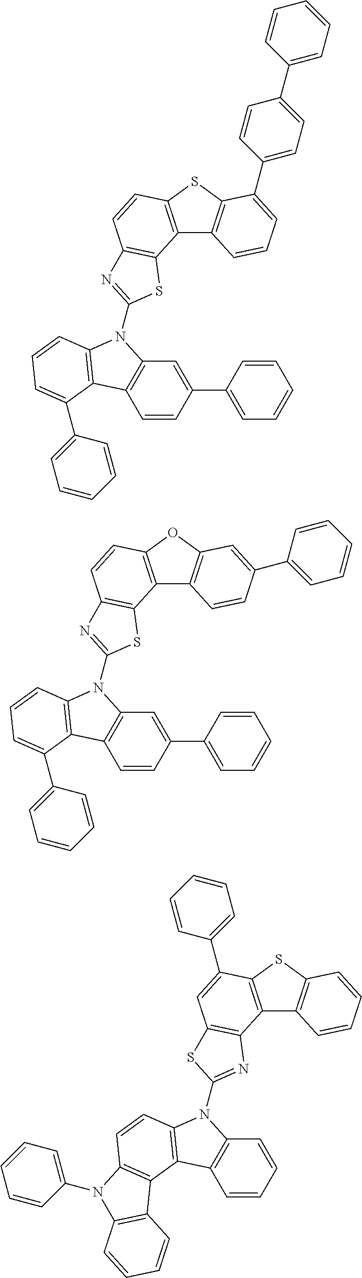 [SMILES: c1ccc(-c2ccc(-c3cccc4c3sc3ccc5nc(-n6c7cc(-c8ccccc8)ccc7c7c(-c8ccccc8)cccc76)sc5c34)cc2)cc1 ; c1ccc(-c2ccc3c(c2)oc2ccc4nc(-n5c6cc(-c7ccccc7)ccc6c6c(-c7ccccc7)cccc65)sc4c23)cc1 ; c1ccc(-c2cc3sc(-n4c5ccccc5c5c6c7ccccc7n(-c7ccccc7)c6ccc54)nc3c3c2sc2ccccc23)cc1]

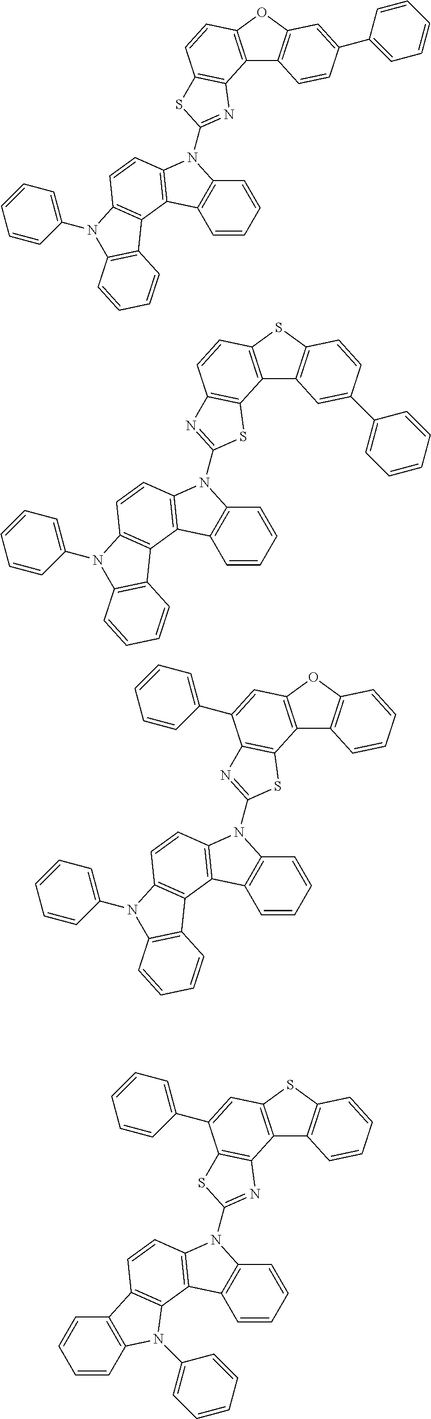

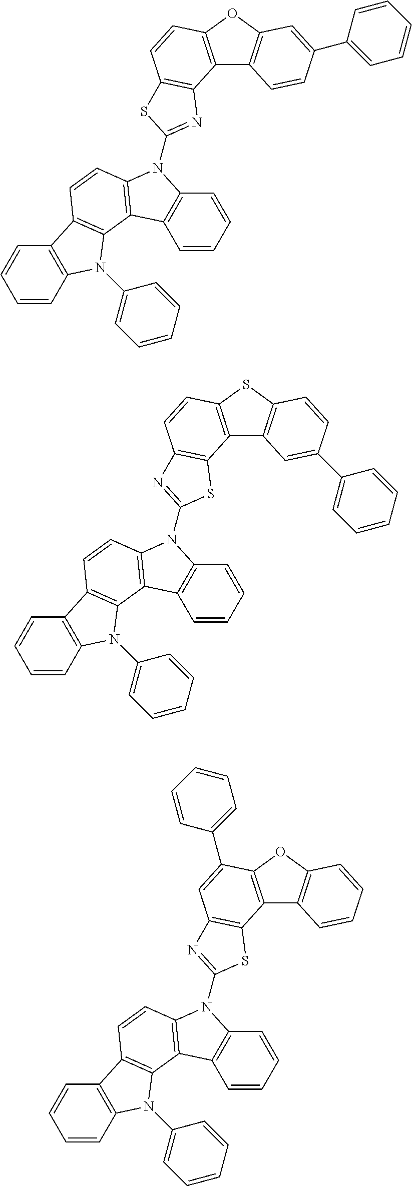 [SMILES: c1ccc(-c2ccc3c(c2)oc2ccc4sc(-n5c6ccccc6c6c5ccc5c7ccccc7n(-c7ccccc7)c56)nc4c23)cc1 ; c1ccc(-c2ccc3sc4ccc5nc(-n6c7ccccc7c7c6ccc6c8ccccc8n(-c8ccccc8)c67)sc5c4c3c2)cc1 ; c1ccc(-c2cc3nc(-n4c5ccccc5c5c4ccc4c6ccccc6n(-c6ccccc6)c45)sc3c3c2oc2ccccc23)cc1]

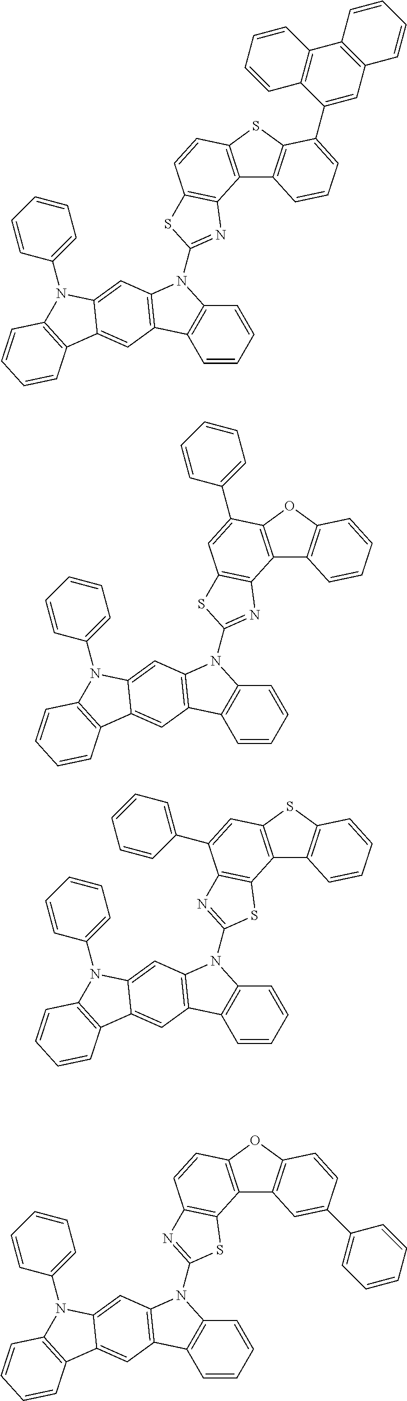 [SMILES: c1ccc(-n2c3ccccc3c3cc4c5ccccc5n(-c5nc6c(ccc7sc8c(-c9cc%10ccccc%10c%10ccccc9%10)cccc8c76)s5)c4cc32)cc1 ; c1ccc(-c2cc3sc(-n4c5ccccc5c5cc6c7ccccc7n(-c7ccccc7)c6cc54)nc3c3c2oc2ccccc23)cc1 ; c1ccc(-c2cc3sc4ccccc4c3c3sc(-n4c5ccccc5c5cc6c7ccccc7n(-c7ccccc7)c6cc54)nc23)cc1 ; c1ccc(-c2ccc3oc4ccc5nc(-n6c7ccccc7c7cc8c9ccccc9n(-c9ccccc9)c8cc76)sc5c4c3c2)cc1]

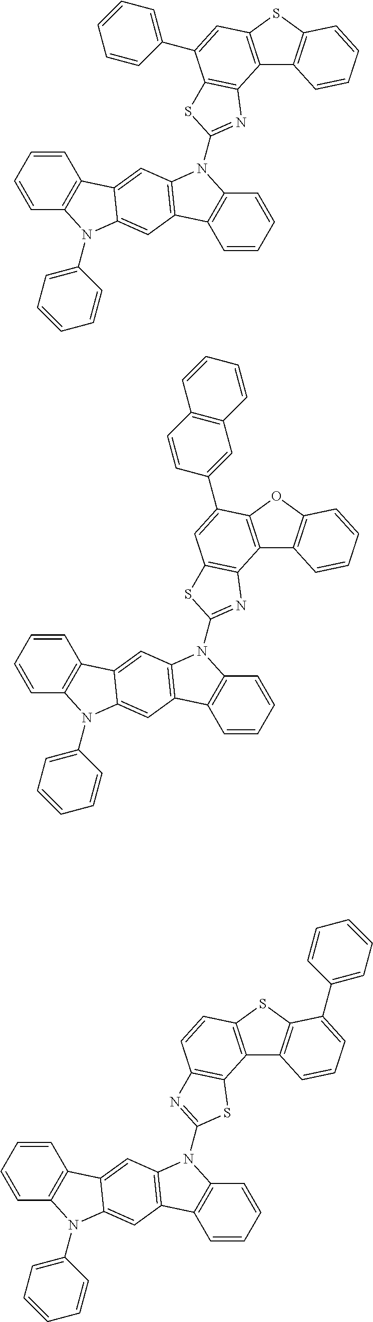 [SMILES: c1ccc(-c2cc3sc4ccccc4c3c3nc(-n4c5ccccc5c5cc6c(cc54)c4ccccc4n6-c4ccccc4)sc23)cc1 ; c1ccc(-n2c3ccccc3c3cc4c(cc32)c2ccccc2n4-c2nc3c(cc(-c4ccc5ccccc5c4)c4oc5ccccc5c43)s2)cc1 ; c1ccc(-c2cccc3c2sc2ccc4nc(-n5c6ccccc6c6cc7c(cc65)c5ccccc5n7-c5ccccc5)sc4c23)cc1]

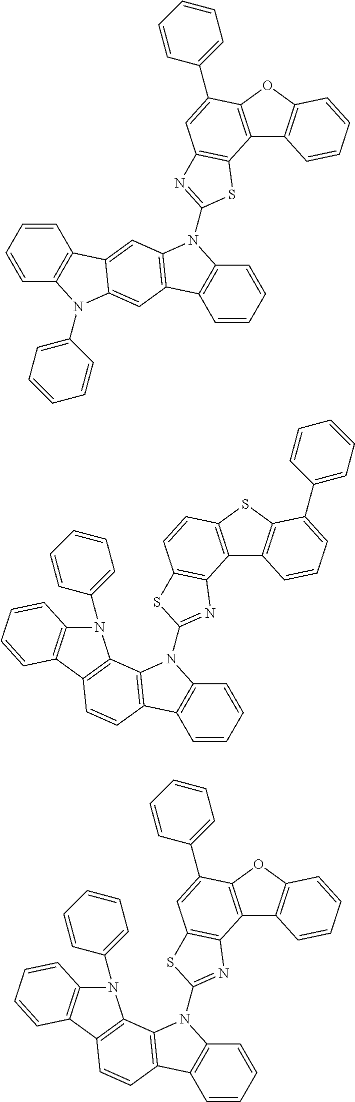

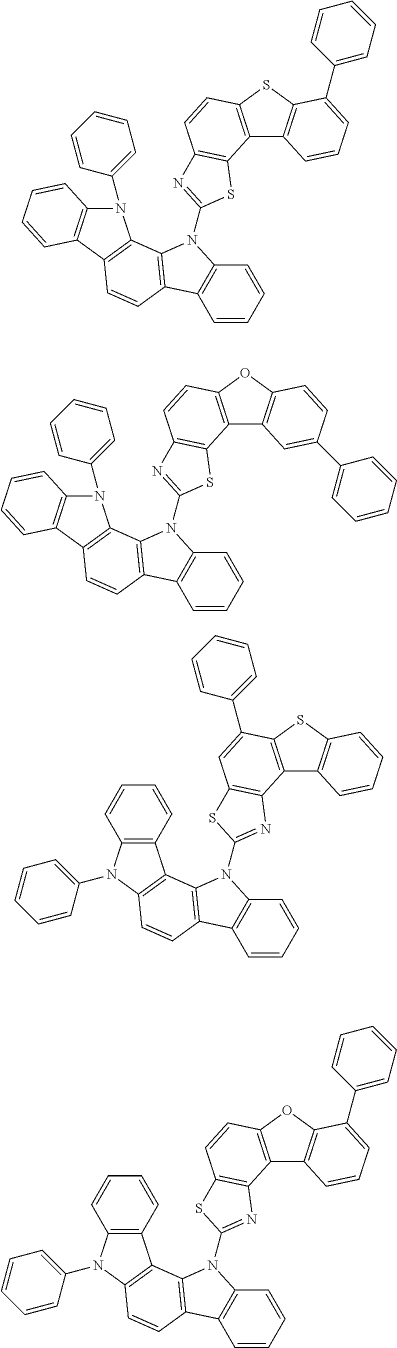

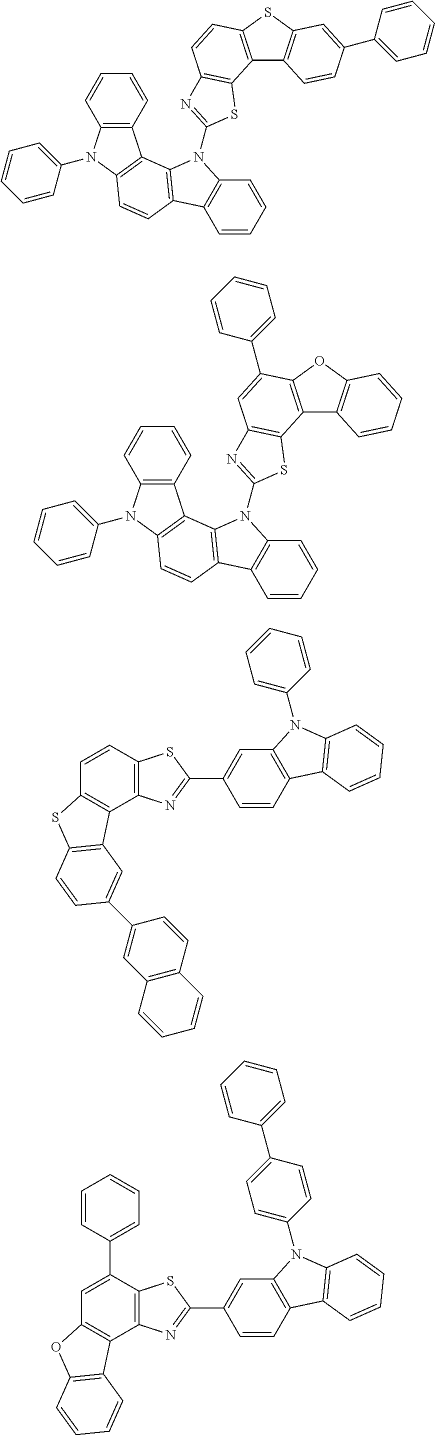

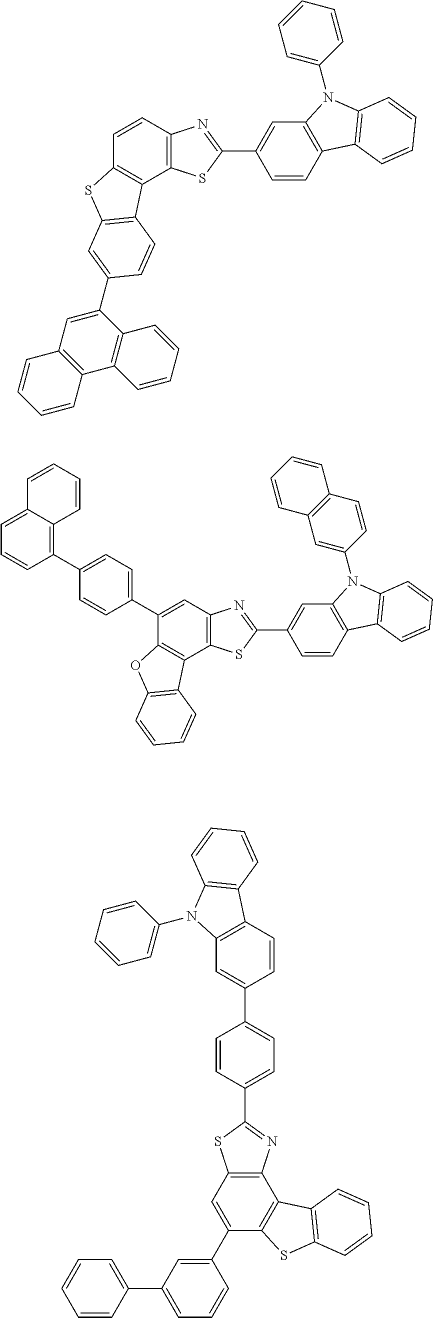 [SMILES: c1ccc(-n2c3ccccc3c3ccc(-c4nc5ccc6sc7cc(-c8cc9ccccc9c9ccccc89)ccc7c6c5s4)cc32)cc1 ; c1ccc2cc(-n3c4ccccc4c4ccc(-c5nc6cc(-c7ccc(-c8cccc9ccccc89)cc7)c7oc8ccccc8c7c6s5)cc43)ccc2c1 ; c1ccc(-c2cccc(-c3cc4sc(-c5ccc(-c6ccc7c8ccccc8n(-c8ccccc8)c7c6)cc5)nc4c4c3sc3ccccc34)c2)cc1]

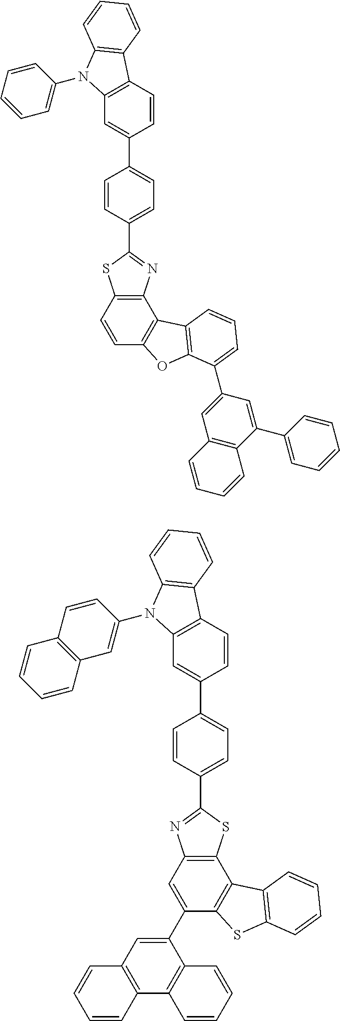 [SMILES: c1ccc(-c2cc(-c3cccc4c3oc3ccc5sc(-c6ccc(-c7ccc8c9ccccc9n(-c9ccccc9)c8c7)cc6)nc5c34)cc3ccccc23)cc1 ; c1ccc2cc(-n3c4ccccc4c4ccc(-c5ccc(-c6nc7cc(-c8cc9ccccc9c9ccccc89)c8sc9ccccc9c8c7s6)cc5)cc43)ccc2c1]

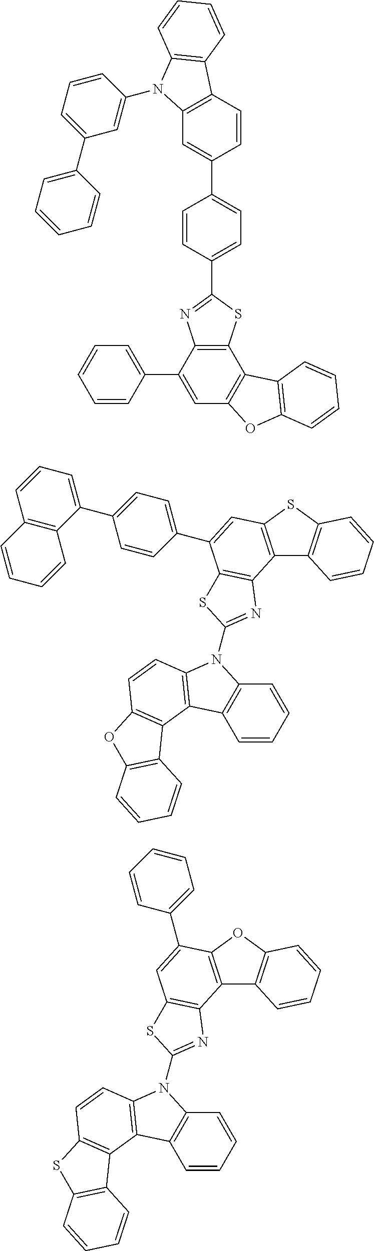

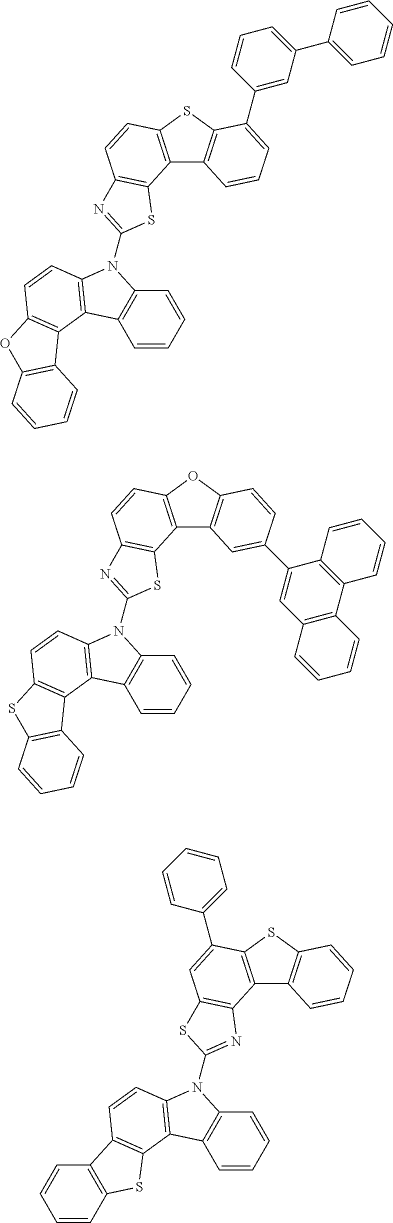

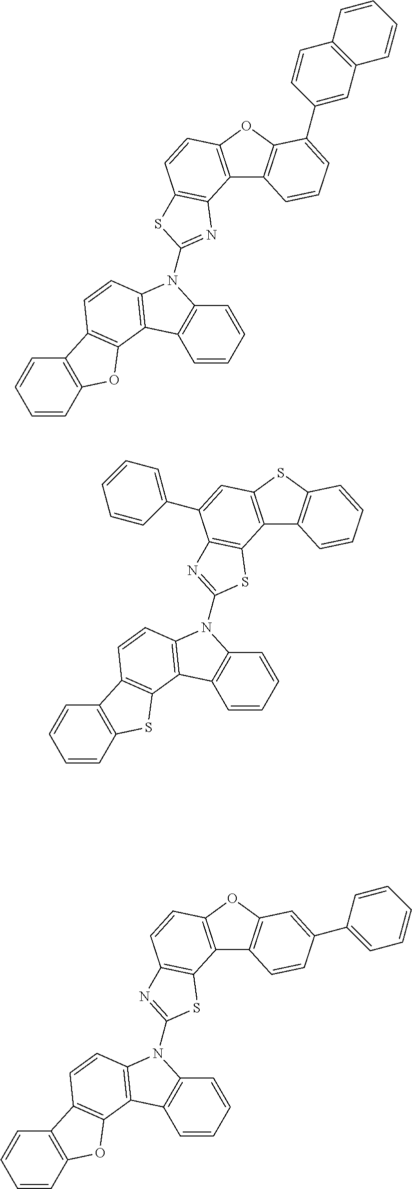

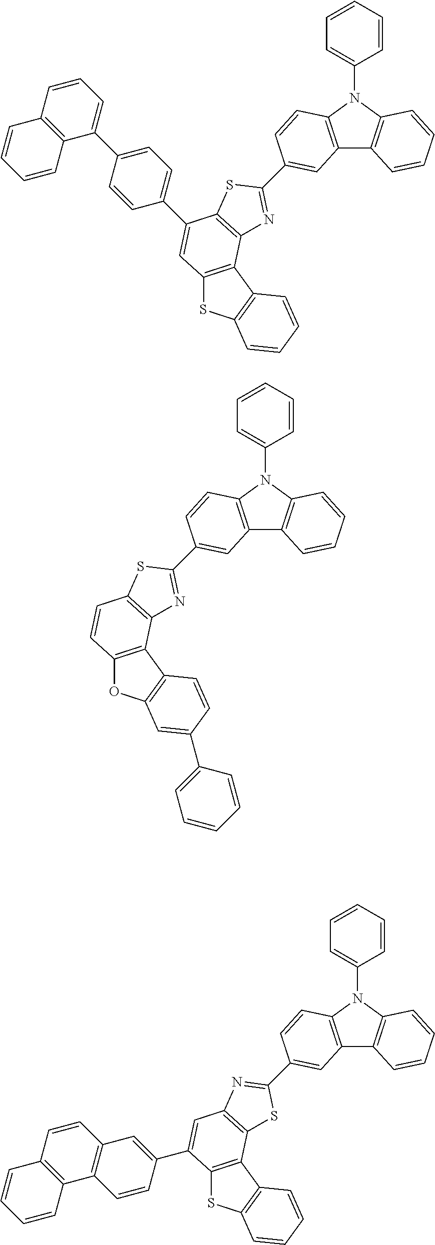

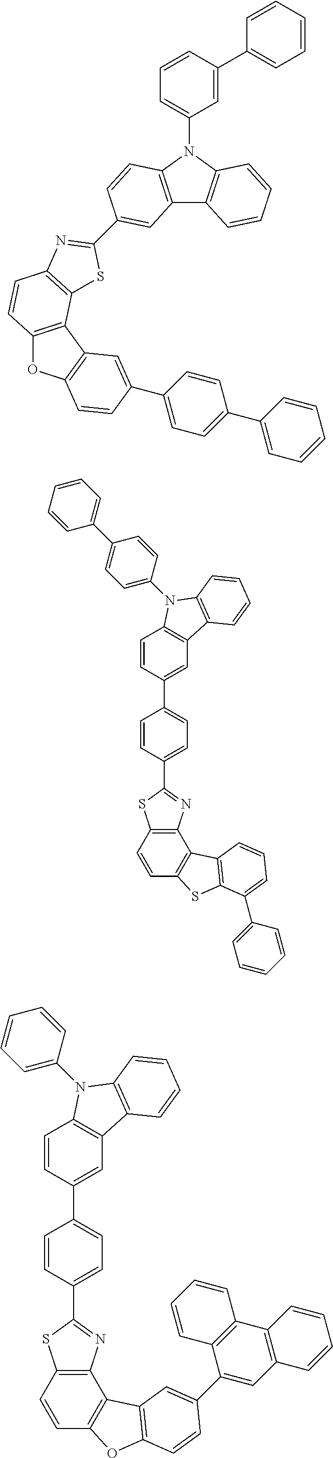 [SMILES: c1ccc(-c2ccc(-c3ccc4oc5ccc6nc(-c7ccc8c(c7)c7ccccc7n8-c7cccc(-c8ccccc8)c7)sc6c5c4c3)cc2)cc1 ; c1ccc(-c2ccc(-n3c4ccccc4c4cc(-c5ccc(-c6nc7c(ccc8sc9c(-c%10ccccc%10)cccc9c87)s6)cc5)ccc43)cc2)cc1 ; c1ccc(-n2c3ccccc3c3cc(-c4ccc(-c5nc6c(ccc7oc8ccc(-c9cc%10ccccc%10c%10ccccc9%10)cc8c76)s5)cc4)ccc32)cc1]

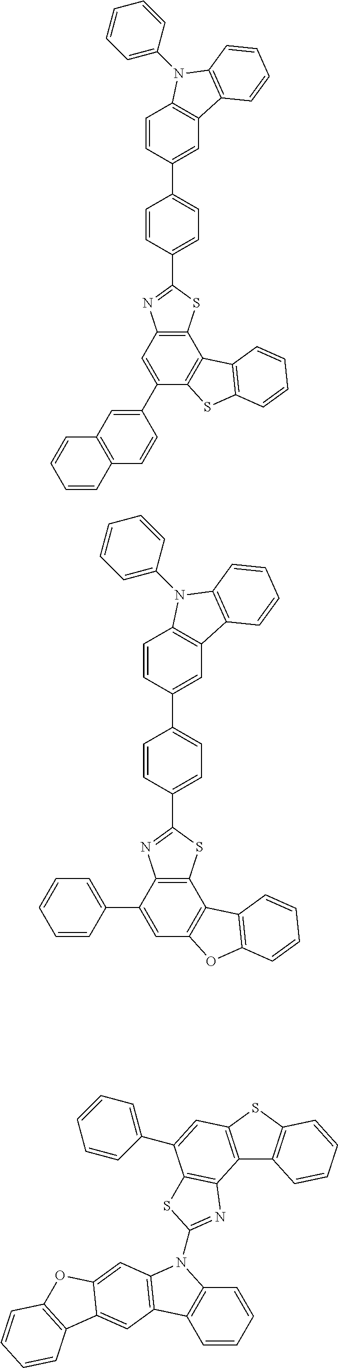

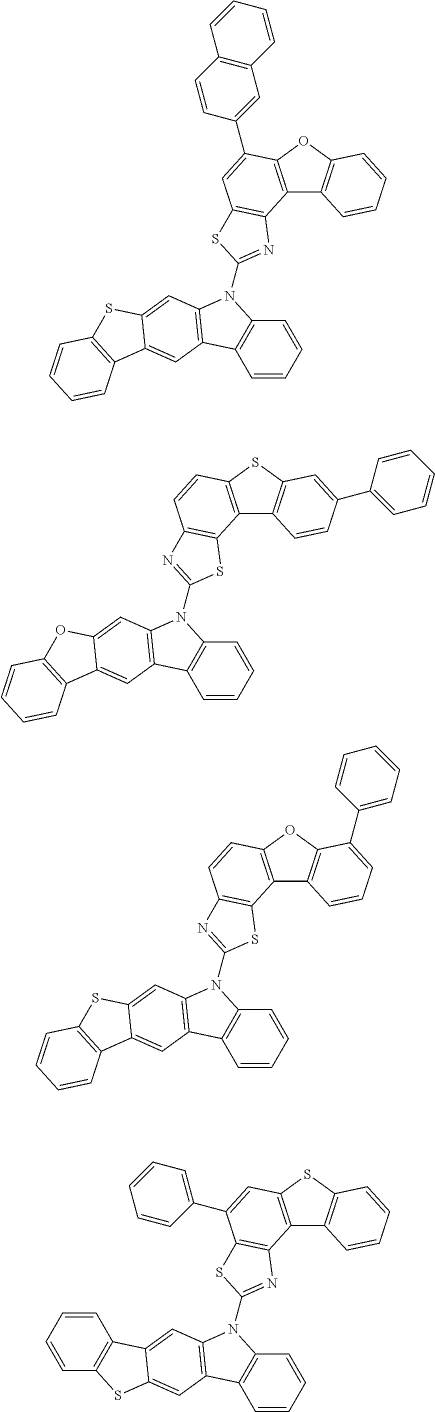 [SMILES: c1ccc2cc(-c3cc4sc(-n5c6ccccc6c6cc7c(cc65)sc5ccccc57)nc4c4c3oc3ccccc34)ccc2c1 ; c1ccc(-c2ccc3c(c2)sc2ccc4nc(-n5c6ccccc6c6cc7c(cc65)oc5ccccc57)sc4c23)cc1 ; c1ccc(-c2cccc3c2oc2ccc4nc(-n5c6ccccc6c6cc7c(cc65)sc5ccccc57)sc4c23)cc1 ; c1ccc(-c2cc3sc4ccccc4c3c3nc(-n4c5ccccc5c5cc6sc7ccccc7c6cc54)sc23)cc1]

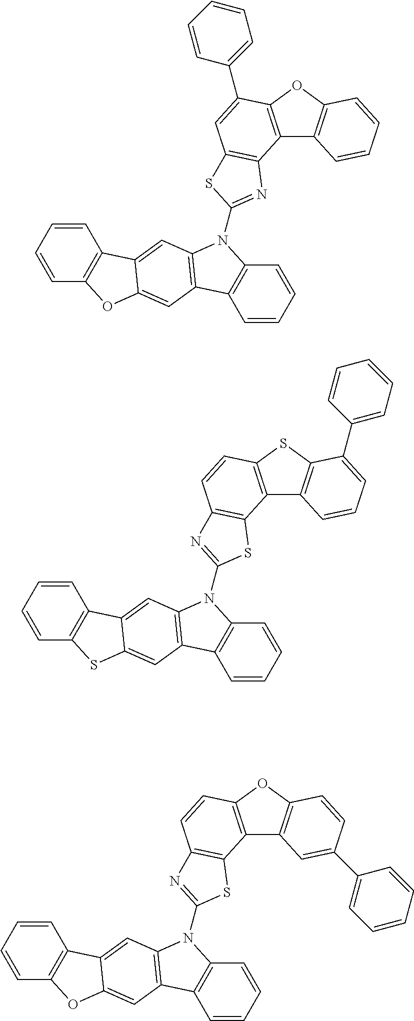 [SMILES: c1ccc(-c2cc3sc(-n4c5ccccc5c5cc6oc7ccccc7c6cc54)nc3c3c2oc2ccccc23)cc1 ; c1ccc(-c2cccc3c2sc2ccc4nc(-n5c6ccccc6c6cc7sc8ccccc8c7cc65)sc4c23)cc1 ; c1ccc(-c2ccc3oc4ccc5nc(-n6c7ccccc7c7cc8oc9ccccc9c8cc76)sc5c4c3c2)cc1]

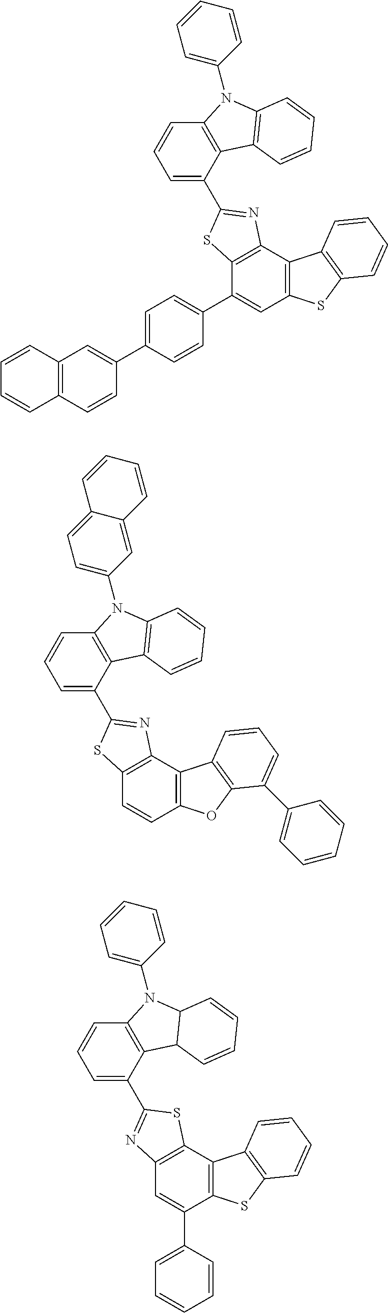 [SMILES: c1ccc(-n2c3ccccc3c3c(-c4nc5c(s4)c(-c4ccc(-c6ccc7ccccc7c6)cc4)cc4sc6ccccc6c45)cccc32)cc1 ; c1ccc(-c2cccc3c2oc2ccc4sc(-c5cccc6c5c5ccccc5n6-c5ccc6ccccc6c5)nc4c23)cc1 ; C1=CC2c3c(-c4nc5cc(-c6ccccc6)c6sc7ccccc7c6c5s4)cccc3N(c3ccccc3)C2C=C1]

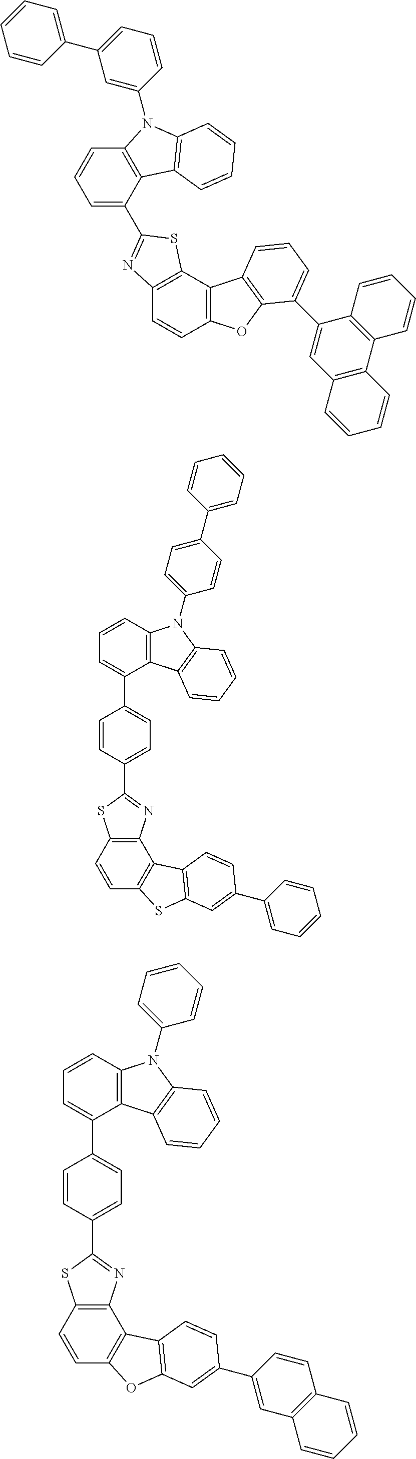 [SMILES: c1ccc(-c2cccc(-n3c4ccccc4c4c(-c5nc6ccc7oc8c(-c9cc%10ccccc%10c%10ccccc9%10)cccc8c7c6s5)cccc43)c2)cc1 ; c1ccc(-c2ccc(-n3c4ccccc4c4c(-c5ccc(-c6nc7c(ccc8sc9cc(-c%10ccccc%10)ccc9c87)s6)cc5)cccc43)cc2)cc1 ; c1ccc(-n2c3ccccc3c3c(-c4ccc(-c5nc6c(ccc7oc8cc(-c9ccc%10ccccc%10c9)ccc8c76)s5)cc4)cccc32)cc1]

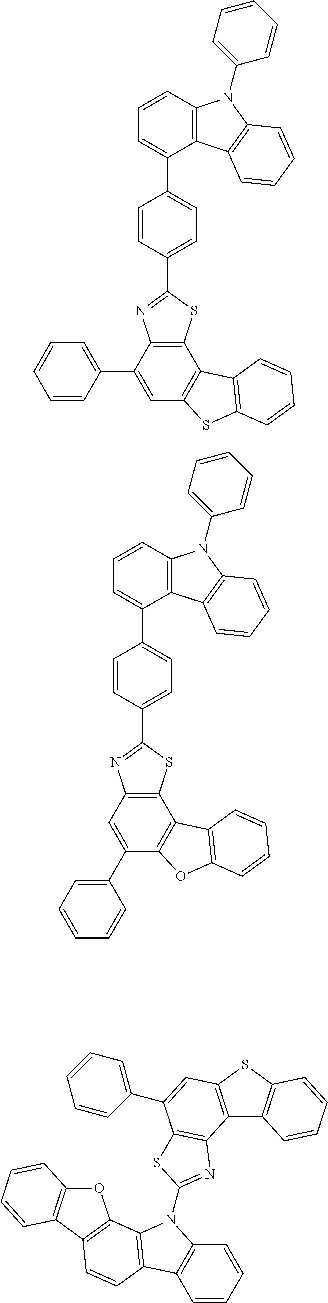

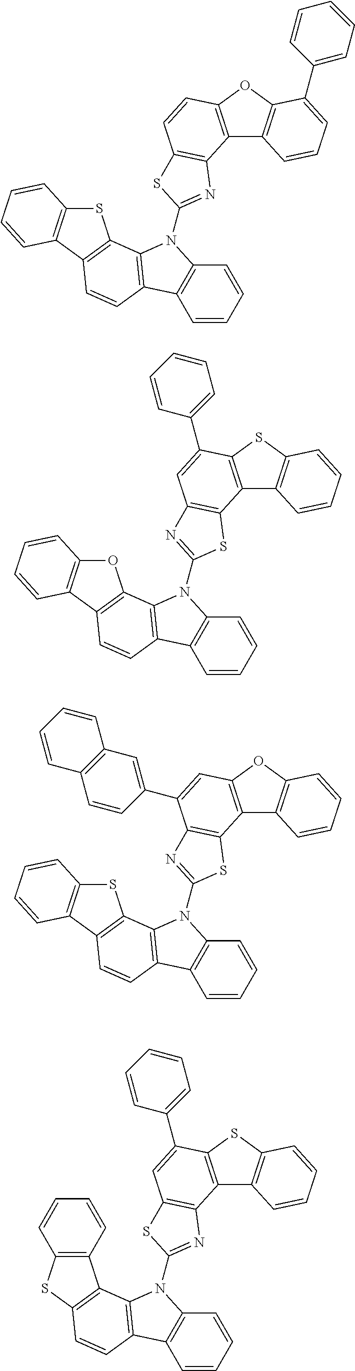

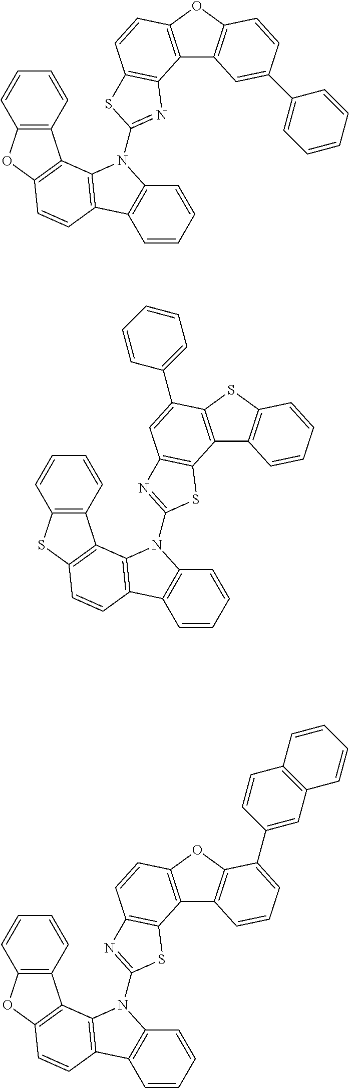 [SMILES: c1ccc(-c2ccc3oc4ccc5sc(-n6c7ccccc7c7ccc8oc9ccccc9c8c76)nc5c4c3c2)cc1 ; c1ccc(-c2cc3nc(-n4c5ccccc5c5ccc6sc7ccccc7c6c54)sc3c3c2sc2ccccc23)cc1 ; c1ccc2cc(-c3cccc4c3oc3ccc5nc(-n6c7ccccc7c7ccc8oc9ccccc9c8c76)sc5c34)ccc2c1]

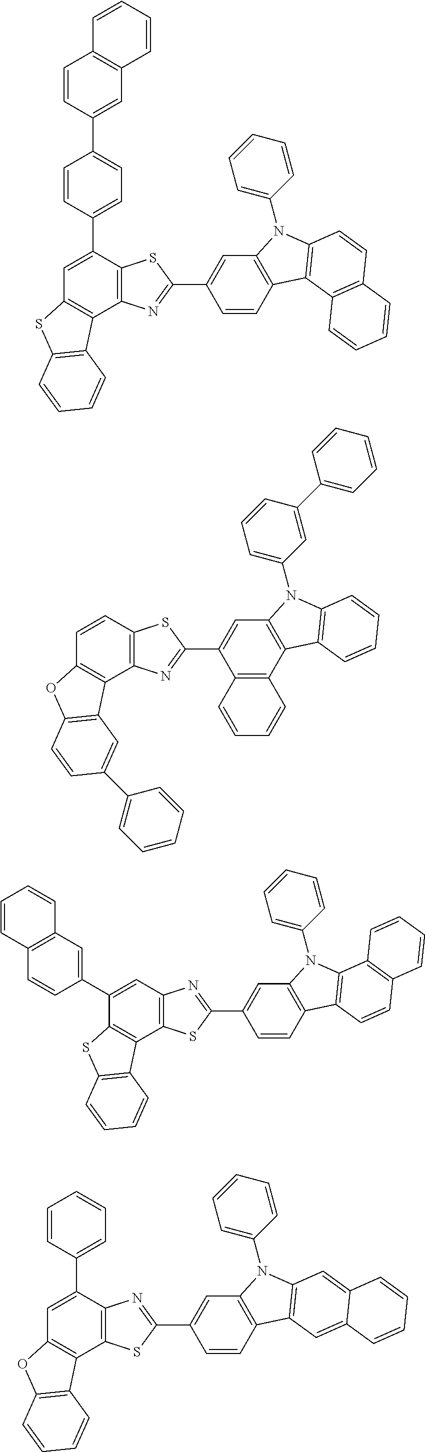 [SMILES: c1ccc(-n2c3cc(-c4nc5c(s4)c(-c4ccc(-c6ccc7ccccc7c6)cc4)cc4sc6ccccc6c45)ccc3c3c4ccccc4ccc32)cc1 ; c1ccc(-c2cccc(-n3c4ccccc4c4c5ccccc5c(-c5nc6c(ccc7oc8ccc(-c9ccccc9)cc8c76)s5)cc43)c2)cc1 ; c1ccc(-n2c3cc(-c4nc5cc(-c6ccc7ccccc7c6)c6sc7ccccc7c6c5s4)ccc3c3ccc4ccccc4c32)cc1 ; c1ccc(-c2cc3oc4ccccc4c3c3sc(-c4ccc5c6cc7ccccc7cc6n(-c6ccccc6)c5c4)nc23)cc1]

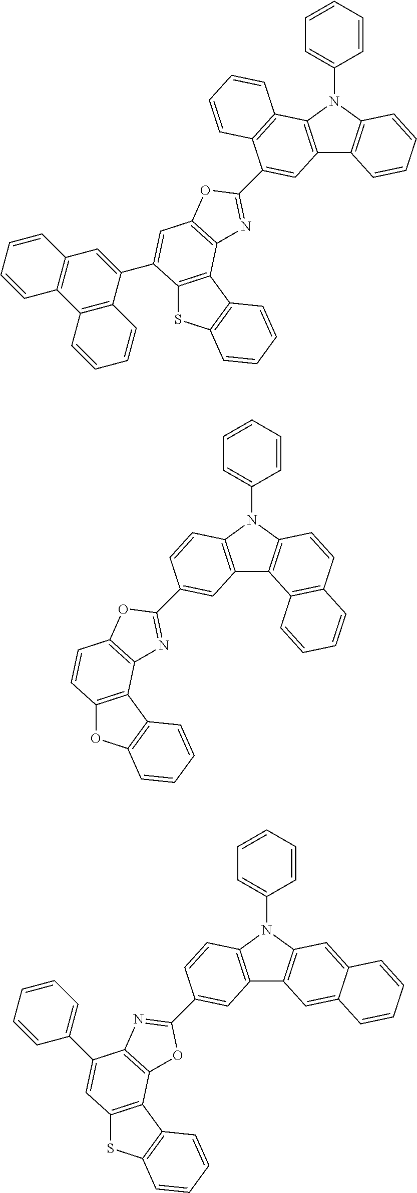 [SMILES: c1ccc(-n2c3ccccc3c3cc(-c4nc5c(cc(-c6cc7ccccc7c7ccccc67)c6sc7ccccc7c65)o4)c4ccccc4c32)cc1 ; c1ccc(-n2c3ccc(-c4nc5c(ccc6oc7ccccc7c65)o4)cc3c3c4ccccc4ccc32)cc1 ; c1ccc(-c2cc3sc4ccccc4c3c3oc(-c4ccc5c(c4)c4cc6ccccc6cc4n5-c4ccccc4)nc23)cc1]

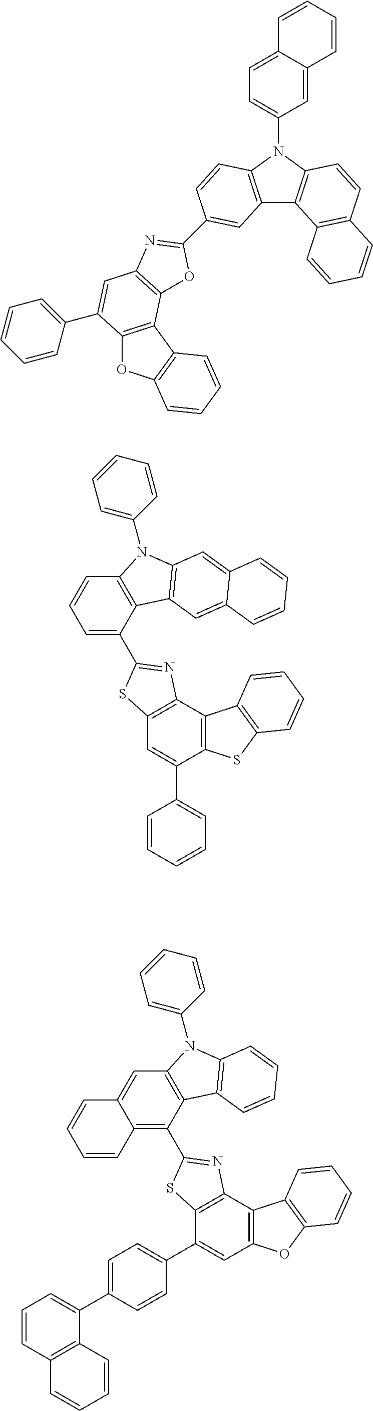 [SMILES: c1ccc(-c2cc3nc(-c4ccc5c(c4)c4c6ccccc6ccc4n5-c4ccc5ccccc5c4)oc3c3c2oc2ccccc23)cc1 ; c1ccc(-c2cc3sc(-c4cccc5c4c4cc6ccccc6cc4n5-c4ccccc4)nc3c3c2sc2ccccc23)cc1 ; c1ccc(-n2c3ccccc3c3c(-c4nc5c(s4)c(-c4ccc(-c6cccc7ccccc67)cc4)cc4oc6ccccc6c45)c4ccccc4cc32)cc1]

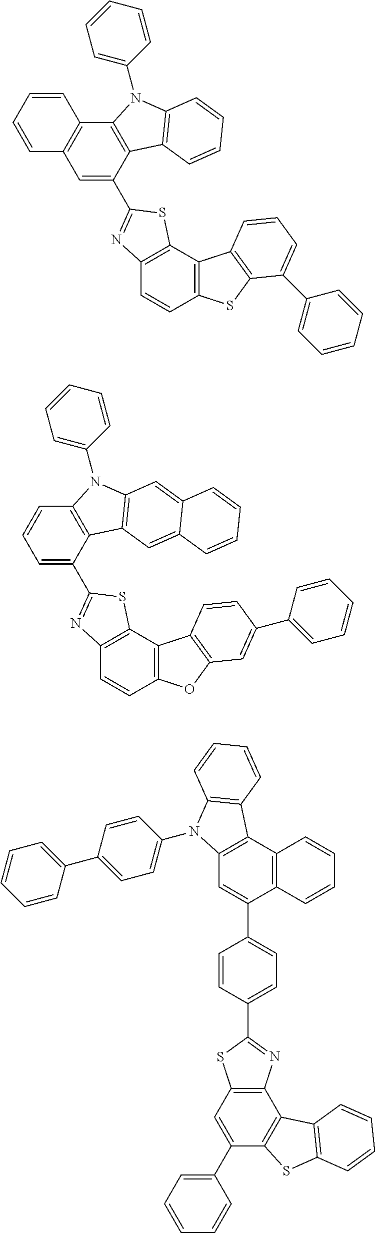 [SMILES: c1ccc(-c2cccc3c2sc2ccc4nc(-c5cc6ccccc6c6c5c5ccccc5n6-c5ccccc5)sc4c23)cc1 ; c1ccc(-c2ccc3c(c2)oc2ccc4nc(-c5cccc6c5c5cc7ccccc7cc5n6-c5ccccc5)sc4c23)cc1 ; c1ccc(-c2ccc(-n3c4ccccc4c4c5ccccc5c(-c5ccc(-c6nc7c(cc(-c8ccccc8)c8sc9ccccc9c87)s6)cc5)cc43)cc2)cc1]

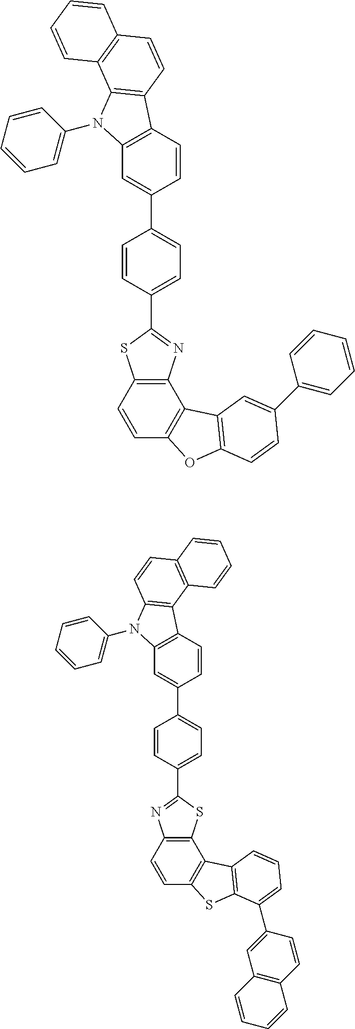 [SMILES: c1ccc(-c2ccc3oc4ccc5sc(-c6ccc(-c7ccc8c9ccc%10ccccc%10c9n(-c9ccccc9)c8c7)cc6)nc5c4c3c2)cc1 ; c1ccc(-n2c3cc(-c4ccc(-c5nc6ccc7sc8c(-c9ccc%10ccccc%10c9)cccc8c7c6s5)cc4)ccc3c3c4ccccc4ccc32)cc1]

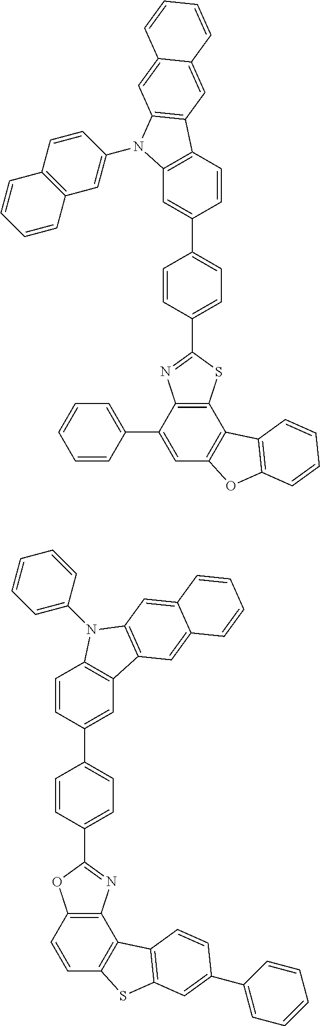 [SMILES: c1ccc(-c2cc3oc4ccccc4c3c3sc(-c4ccc(-c5ccc6c7cc8ccccc8cc7n(-c7ccc8ccccc8c7)c6c5)cc4)nc23)cc1 ; c1ccc(-c2ccc3c(c2)sc2ccc4oc(-c5ccc(-c6ccc7c(c6)c6cc8ccccc8cc6n7-c6ccccc6)cc5)nc4c23)cc1]

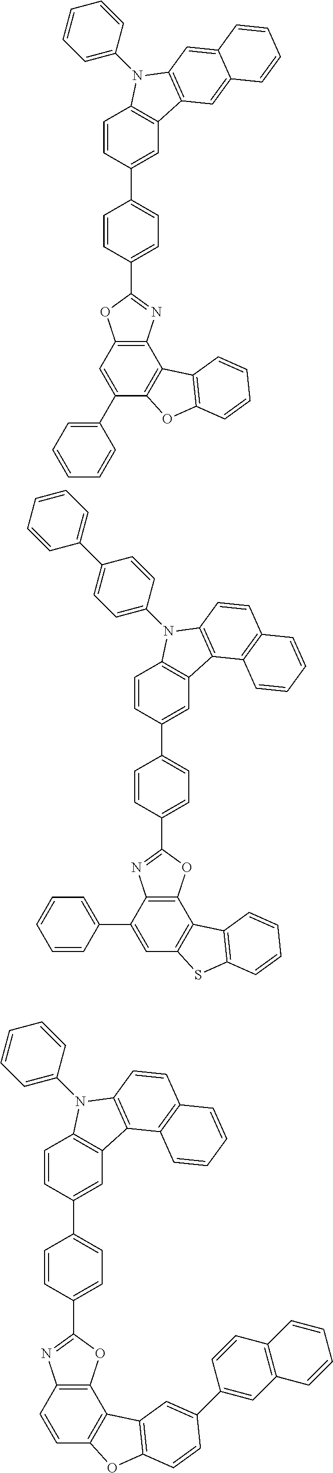

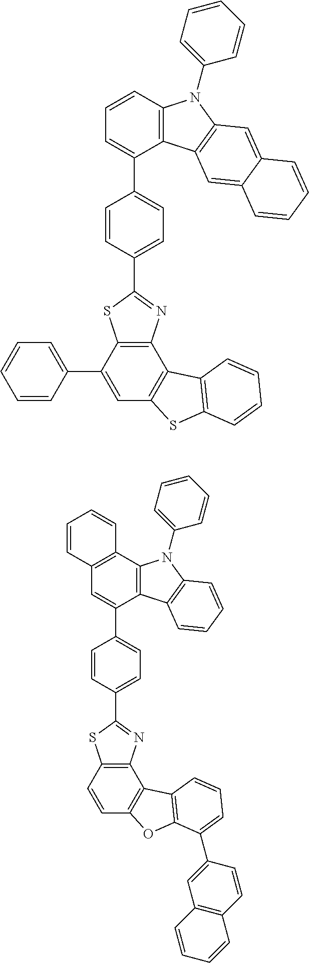 [SMILES: c1ccc(-c2cc3sc4ccccc4c3c3nc(-c4ccc(-c5cccc6c5c5cc7ccccc7cc5n6-c5ccccc5)cc4)sc23)cc1 ; c1ccc(-n2c3ccccc3c3c(-c4ccc(-c5nc6c(ccc7oc8c(-c9ccc%10ccccc%10c9)cccc8c76)s5)cc4)cc4ccccc4c32)cc1]

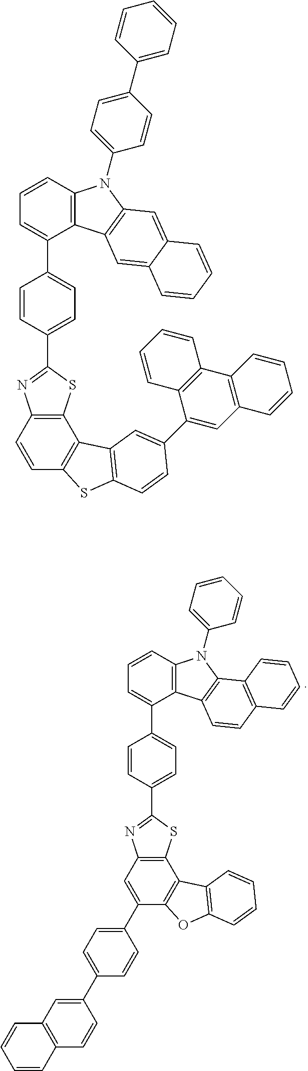 [SMILES: c1ccc(-c2ccc(-n3c4cc5ccccc5cc4c4c(-c5ccc(-c6nc7ccc8sc9ccc(-c%10cc%11ccccc%11c%11ccccc%10%11)cc9c8c7s6)cc5)cccc43)cc2)cc1 ; c1ccc(-n2c3cccc(-c4ccc(-c5nc6cc(-c7ccc(-c8ccc9ccccc9c8)cc7)c7oc8ccccc8c7c6s5)cc4)c3c3ccc4ccccc4c32)cc1 ; "."]

In addition, the present disclosure provides a method for preparing the compound of Chemical Formula 1 or Chemical Formula 2, as shown in the following Reaction Scheme 1 or Reaction Scheme 2:

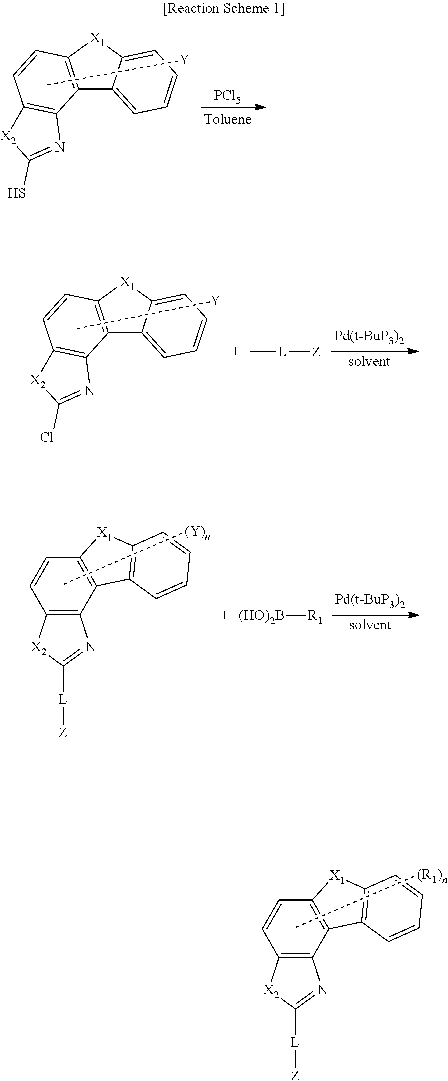

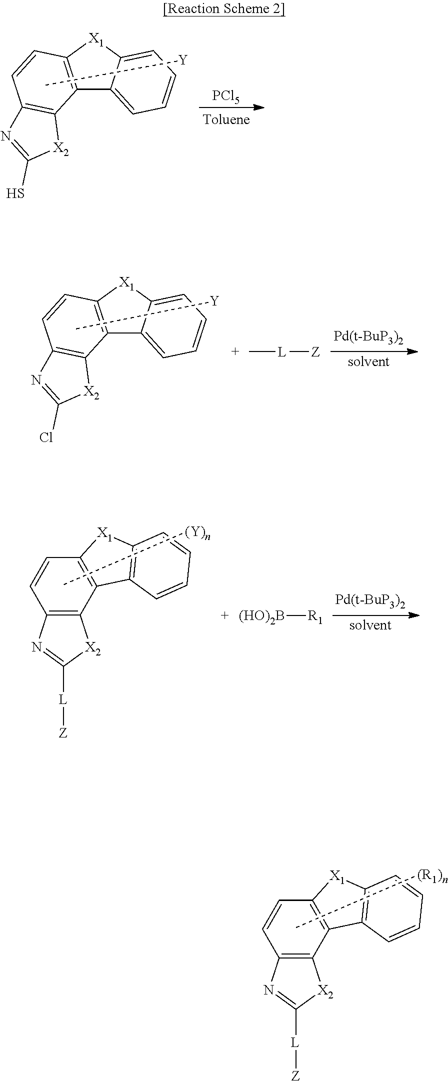

In Reaction Schemes 1 and 2, the remaining substituents except for Y are the same as defined above, and Y is halogen, preferably bromo or chloro.

The reaction is preferably carried out in the presence of a palladium catalyst and a base, and a reactive group for the reaction can be changed as known in the art. The above preparation method can be further embodied in Preparation Examples described hereinafter.

(Organic Light Emitting Device)

In another embodiment of the present disclosure, provided is an organic light emitting device including the compound of Chemical Formula 1 or Chemical Formula 2. In one example, the present disclosure provides an organic light emitting device comprising: a first electrode; a second electrode that is opposite to the first electrode; and one or more organic material layers that are between the first electrode and the second electrode, wherein one or more layers of the organic material layers include the compound of Chemical Formula 1 or Chemical Formula 2.

The organic material layer of the organic light emitting device of the present disclosure can have a single-layer structure, or it can have a multilayered structure in which two or more organic material layers are stacked. For example, the organic light emitting device of the present disclosure can have a structure comprising a hole injection layer, a hole transport layer, a light emitting layer, an electron transport layer, an electron blocking layer, an electron injection layer and the like as the organic material layer. However, the structure of the organic light emitting device is not limited thereto, and it can include a smaller number of organic layers.

Further, the organic material layer can include a hole injection layer, a hole transport layer, or a layer for simultaneously performing hole injection and hole transport, and the hole injection layer, the hole transport layer, or the layer for simultaneously performing hole injection and hole transport can include the compound of Chemical Formula 1 or Chemical Formula 2.

Further, the organic material layer can include a light emitting layer, wherein the light emitting layer can include the compound of Chemical Formula 1 or Chemical Formula 2. Particularly, the compound according to the present disclosure can be used as a dopant of the light emitting layer.

Further, the organic material layer can include an electron transport layer or an electron injection layer, and the electron transport layer or the electron injection layer can include a compound of Chemical Formula 1 or Chemical Formula 2.

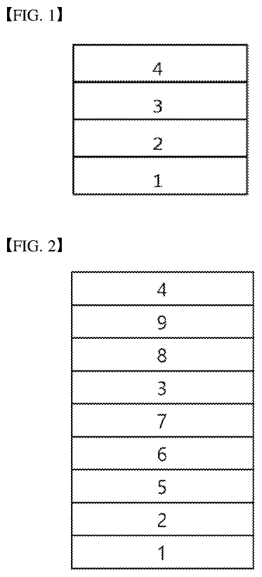

Further, the organic light emitting device according to the present disclosure can be a normal type organic light emitting device in which an anode, one or more organic material layers and a cathode are sequentially stacked on a substrate. Further, the organic light emitting device according to the present disclosure can be an inverted type organic light emitting device in which a cathode, one or more organic material layers and an anode are sequentially stacked on a substrate. For example, the structure of an organic light emitting device according to an embodiment of the present disclosure is illustrated in FIGS. 1 and 2.

FIG. 1 shows an example of an organic light emitting device comprising a substrate 1, an anode 2, a light emitting layer 3, and a cathode 4. In such a structure, the compound of Chemical Formula 1 can be included in the light emitting layer.

FIG. 2 shows an example of an organic light emitting device comprising a substrate 1, an anode 2, a hole injection layer 5, a hole transport layer 6, an electron blocking layer 7, a light emitting layer 3, an electron transport layer 8, an electron injection layer 9, and a cathode 4. In such a structure, the compound of Chemical Formula 1 or Chemical Formula 2 can be included in the light emitting layer.

The organic light emitting device according to the present disclosure can be manufactured by materials and methods known in the art, except that at least one of the organic material layers includes the compound of Chemical Formula 1 or Chemical Formula 2. Further, when the organic light emitting device includes a plurality of organic material layers, the organic material layers can be formed of the same material or different materials.

For example, the organic light emitting device according to the present disclosure can be manufactured by sequentially stacking a first electrode, an organic material layer and a second electrode on a substrate. In this case, the organic light emitting device can be manufactured by depositing a metal, metal oxides having conductivity, or an alloy thereof on the substrate using a PVD (physical vapor deposition) method such as a sputtering method or an e-beam evaporation method to form an anode, forming organic material layers including the hole injection layer, the hole transport layer, the light emitting layer and the electron transport layer thereon, and then depositing a material that can be used as the cathode thereon. In addition to such a method, the organic light emitting device can be manufactured by sequentially depositing the cathode material, the organic material layer and the anode material on a substrate.

Further, the compound of Chemical Formula 1 or Chemical Formula 2 can be formed into an organic material layer by a solution coating method as well as a vacuum deposition method when manufacturing an organic light emitting device. Herein, the solution coating method means a spin coating, a dip coating, a doctor blading, an inkjet printing, a screen printing, a spray method, a roll coating, or the like, but is not limited thereto.

In addition to such a method, the organic light emitting device can be manufactured by sequentially depositing a cathode material, an organic material layer and an anode material on a substrate (International Publication WO2003/012890). However, the manufacturing method is not limited thereto.

In one example, the first electrode is an anode, and the second electrode is a cathode, or alternatively, the first electrode is a cathode and the second electrode is an anode.

As the anode material, generally, a material having a large work function is preferably used so that holes can be smoothly injected into the organic material layer. Specific examples of the anode material include metals such as vanadium, chrome, copper, zinc, and gold, or an alloy thereof; metal oxides such as zinc oxides, indium oxides, indium tin oxides (ITO), and indium zinc oxides (IZO); a combination of metals and oxides, such as ZnO:Al or SnO₂:Sb; conductive polymers such as poly(3-methylthiophene), poly[3,4-(ethylene-1,2-dioxy)thiophene](PEDOT), polypyrrole, and polyaniline, and the like, but are not limited thereto.

As the cathode material, generally, a material having a small work function is preferably used so that electrons can be easily injected into the organic material layer. Specific examples of the cathode material include metals such as magnesium, calcium, sodium, potassium, titanium, indium, yttrium, lithium, gadolinium, aluminum, silver, tin, and lead, or an alloy thereof; a multilayered structure material such as LiF/Al or LiO₂/Al, and the like, but are not limited thereto.

The hole injection layer is a layer for injecting holes from the electrode, and the hole injection material is preferably a compound which has a capability of transporting the holes, thus has a hole injecting effect in the anode and an excellent hole injecting effect to the light emitting layer or the light emitting material, prevents excitons produced in the light emitting layer from moving to an electron injection layer or the electron injection material, and further is excellent in the ability to form a thin film. It is preferable that a HOMO (highest occupied molecular orbital) of the hole injection material is between the work function of the anode material and a HOMO of a peripheral organic material layer. Specific examples of the hole injection material include metal porphyrin, oligothiophene, an arylamine-based organic material, a hexanitrilehexaazatriphenylene-based organic material, a quinacridone-based organic material, a perylene-based organic material, anthraquinone, polyaniline and polythiophene-based conductive polymer, and the like, but are not limited thereto.

The hole transport layer is a layer that receives holes from a hole injection layer and transports the holes to the light emitting layer. The hole transport material is suitably a material having large mobility to the holes, which can receive holes from the anode or the hole injection layer and transfer the holes to the light emitting layer. Specific examples thereof include an arylamine-based organic material, a conductive compound, a block copolymer in which a conjugate portion and a non-conjugate portion are present together, and the like, but are not limited thereto.

The light emitting material is preferably a material which can receive holes and electrons transported from a hole transport layer and an electron transport layer, respectively, and combine the holes and the electrons to emit light in a visible ray region, and has good quantum efficiency to fluorescence or phosphorescence. Specific examples of the light emitting material include an 8-hydroxy-quinoline aluminum complex (Alq₃), a carbazole-based compound, a dimerized styryl compound, BAlq, a 10-hydroxybenzoquinoline-metal compound, a benzoxazole, benzthiazole and benzimidazole-based compound, a poly(p-phenylenevinylene) (PPV)-based polymer, a spiro compound, polyfluorene, rubrene, and the like, but are not limited thereto.

The light emitting layer can include a host material and a dopant material. The host material can be a fused aromatic ring derivative, a heterocycle-containing compound or the like. Specific examples of the fused aromatic ring derivatives include anthracene derivatives, pyrene derivatives, naphthalene derivatives, pentacene derivatives, phenanthrene compounds, fluoranthene compounds, and the like. Examples of the heterocyclic-containing compounds include carbazole derivatives, dibenzofuran derivatives, ladder-type furan compounds, pyrimidine derivatives, and the like, but are not limited thereto.

Examples of the dopant material include an aromatic amine derivative, a styrylamine compound, a boron complex, a fluoranthene compound, a metal complex, and the like. Specifically, the aromatic amine derivative is a substituted or unsubstituted fused aromatic ring derivative having an arylamino group, and examples thereof include pyrene, anthracene, chrysene, periflanthene and the like, which have an arylamino group. The styrylamine compound is a compound where at least one arylvinyl group is substituted in substituted or unsubstituted arylamine, in which one or two or more substituent groups selected from the group consisting of an aryl group, a silyl group, an alkyl group, a cycloalkyl group, and an arylamino group are substituted or unsubstituted. Specific examples thereof include styrylamine, styryldiamine, styryltriamine, styryltetramine, and the like, but are not limited thereto. Further, the metal complex includes an iridium complex, a platinum complex, and the like, but is not limited thereto.

The electron transport layer is a layer which receives electrons from an electron injection layer and transports the electrons to a light emitting layer, and an electron transport material is suitably a material which can receive electrons well from a cathode and transfer the electrons to a light emitting layer, and has a large mobility for electrons. Specific examples of the electron transport material include: an Al complex of 8-hydroxyquinoline; a complex including Alq₃; an organic radical compound; a hydroxyflavone-metal complex, and the like, but are not limited thereto. The electron transport layer can be used together with any desired cathode material, as used according to the related art. In particular, appropriate examples of the cathode material are a typical material which has a low work function, followed by an aluminum layer or a silver layer. Specific examples thereof include cesium, barium, calcium, ytterbium, and samarium, in each case followed by an aluminum layer or a silver layer.

The electron injection layer is a layer which injects electrons from an electrode, and is preferably a compound which has a capability of transporting electrons, has an effect of injecting electrons from a cathode and an excellent effect of injecting electrons into a light emitting layer or a light emitting material, prevents excitons produced from the light emitting layer from moving to a hole injection layer, and is also excellent in the ability to form a thin film. Specific examples of the electron injection layer include fluorenone, anthraquinodimethane, diphenoquinone, thiopyran dioxide, oxazole, oxadiazole, triazole, imidazole, perylenetetracarboxylic acid, fluorenylidene methane, anthrone, and the like, and derivatives thereof, a metal complex compound, a nitrogen-containing 5-membered ring derivative, and the like, but are not limited thereto. Preferably, the compound of Chemical Formula 1 can be included as a material of the electron injection layer.

Examples of the metal complex compound include 8-hydroxyquinolinato lithium, bis(8-hydroxyquinolinato)zinc, bis(8-hydroxyquinolinato)copper, bis(8-hydroxyquinolinato)manganese, tris(8-hydroxyquinolinato)aluminum, tris(2-methyl-8-hydroxyquinolinato)aluminum, tris(8-hydroxyquinolinato)gallium, bis(10-hydroxybenzo[h]quinolinato)beryllium, bis(10-hydroxybenzo[h]quinolinato)zinc, bis(2-methyl-8-quinolinato)chlorogallium, bis(2-methyl-8-quinolinato)(o-cresolato)gallium, bis(2-methyl-8-quinolinato)(1-naphtholato)aluminum, bis(2-methyl-8-quinolinato)(2-naphtholato)gallium, and the like, but are not limited thereto.

The organic light emitting device according to the present disclosure can be a bottom emission device, a top emission device, or a double-sided light emitting device, and in particular, can be a bottom emission device that requires relatively high luminous efficiency.

In addition, the compound according to the present disclosure can be included in an organic solar cell or an organic transistor in addition to an organic light emitting device.

The preparation of the compound of Chemical Formula 1 or Chemical Formula 2 and the organic light emitting device including the same will be specifically described in the following Examples. However, the following Examples are provided for illustrative purposes only, and are not intended to limit the scope of the present disclosure.

### EXAMPLES

### Example 1: Preparation of Compound AA

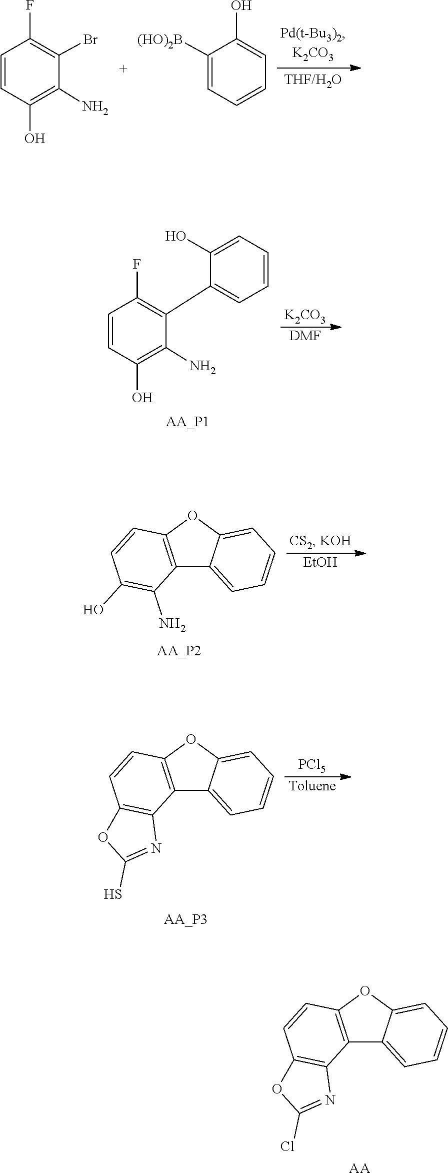

2-Amino-3-bromo-4-fluorophenol (15 g, 72.8 mmol) and (2-hydroxyphenyl)boronic acid (10.5 g, 76.5 mmol) were added to THF (300 ml), and the mixture was stirred and refluxed. Then, potassium carbonate (30.2 g, 218.4 mmol) was dissolved in water (91 ml) and added thereto, and the mixture was sufficiently stirred, and then bis(tri-tert-butylphosphine)palladium(0) (0.4 g, 0.7 mmol) was added. After reacting for 2 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound AA_P1 (11.6 g). (Yield: 73%, MS: [M+H]⁺=220)

Compound AA_P1 (15 g, 68.4 mmol) and potassium carbonate (28.4 g, 205.3 mmol) were added to DMF (150 ml), and the mixture was stirred and refluxed. After reacting for 3 hours, the reaction mixture was cooled to room temperature, and then the organic solvent was distilled under reduced pressure. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound AA_P2 (9.5 g). (Yield: 70%, MS: [M+H]⁺=200)

Compound AA_P2 (15 g, 75.3 mmol), carbon disulfide (8.6 g, 112.9 mmol), and potassium hydroxide (6.3 g, 112.9 mmol) were added to EtOH (150 ml), and the mixture was stirred and refluxed. After reacting for 3 hours, the reaction mixture was cooled to room temperature, and then the organic solvent was distilled under reduced pressure. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound AA_P3 (12.5 g). (Yield: 69%, MS: [M+H]+=242)

Compound AA_P3 (15 g, 62.2 mmol) and phosphorus pentachloride (13.6 g, 65.3 mmol) were added to toluene (150 ml), and the mixture was stirred and refluxed. After reacting for 4 hours, the reaction mixture was cooled to room temperature, and then the organic solvent was distilled under reduced pressure. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound AA (9.7 g). (Yield: 64%, MS: [M+H]+=244)

### Synthesis Scheme of Preparation Examples 2 and 3

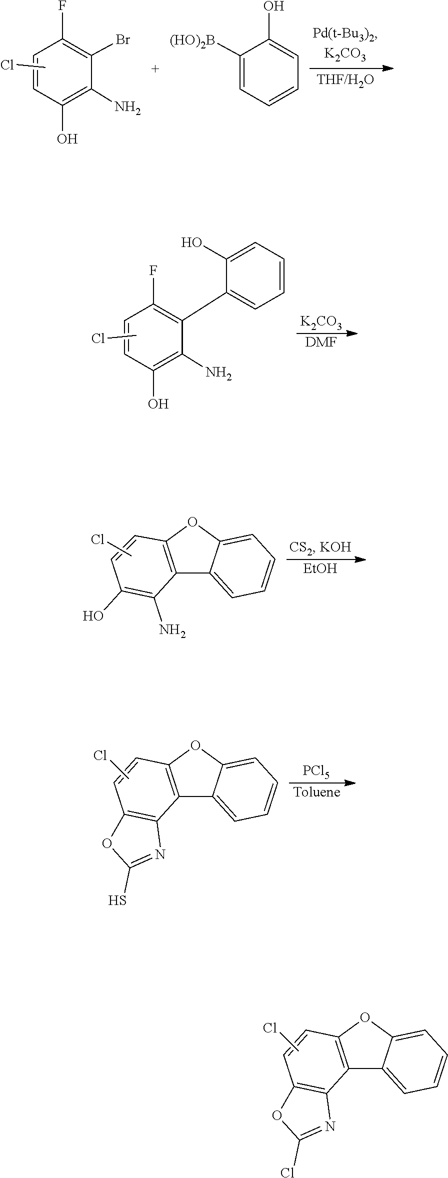

### Preparation Example 2: Preparation of Compound AB

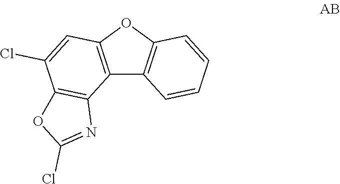 [SMILES: Clc1nc2c(o1)c(Cl)cc1oc3ccccc3c12]

Compound AB was prepared in the same manner as in Preparation Example 1, except that 2-amino-3-bromo-6-chloro-4-fluorophenol was used instead of 2-amino-3-bromo-4-fluorophenol.

### Preparation Example 3: Preparation of Compound AC

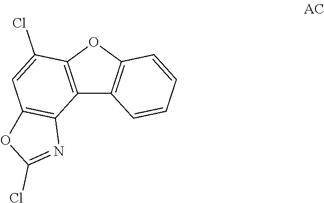 [SMILES: Clc1nc2c(cc(Cl)c3oc4ccccc4c32)o1]

Compound AC was prepared in the same manner as in Preparation Example 1, except that 2-amino-3-bromo-5-chloro-4-fluorophenol was used instead of 2-amino-3-bromo-4-fluorophenol.

### Synthesis Scheme of Preparation Examples 4 to 6

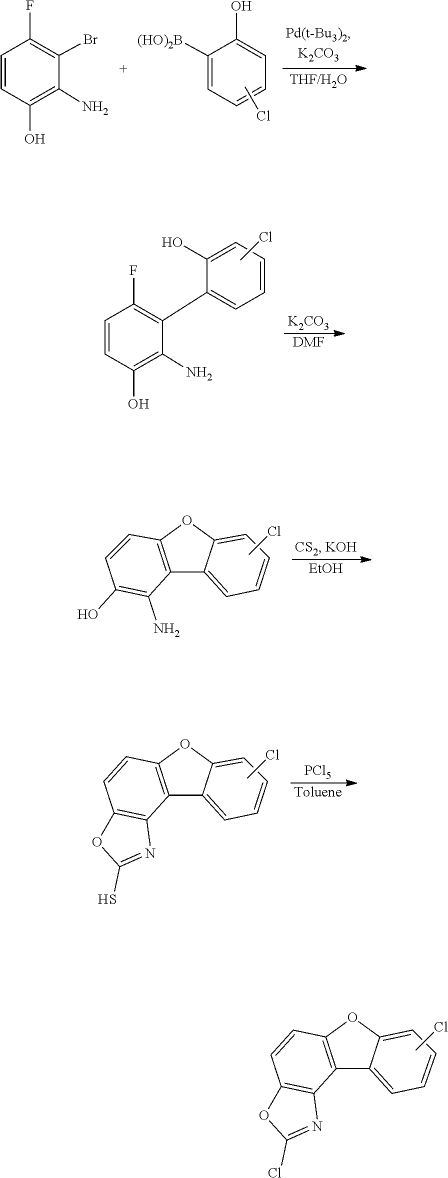

### Preparation Example 4: Preparation of Compound AD

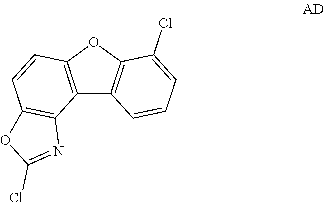 [SMILES: Clc1nc2c(ccc3oc4c(Cl)cccc4c32)o1]

Compound AD was prepared in the same manner as in Preparation Example 1, except that (3-chloro-2-hydroxyphenyl)boronic acid was used instead of (2-hydroxyphenyl)boronic acid.

### Preparation Example 5: Preparation of Compound AE

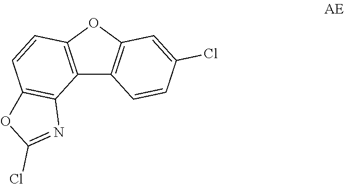 [SMILES: Clc1ccc2c(c1)oc1ccc3oc(Cl)nc3c12]

Compound AE was prepared in the same manner as in Preparation Example 1, except that (4-chloro-2-hydroxyphenyl)boronic acid was used instead of (2-hydroxyphenyl)boronic acid.

### Preparation Example 6: Preparation of Compound AF

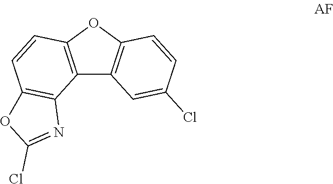 [SMILES: Clc1ccc2oc3ccc4oc(Cl)nc4c3c2c1]

Compound AF was prepared in the same manner as in Preparation Example 1, except that (5-chloro-2-hydroxyphenyl)boronic acid was used instead of (2-hydroxyphenyl)boronic acid.

### Preparation Example 7: Preparation of Compound BA

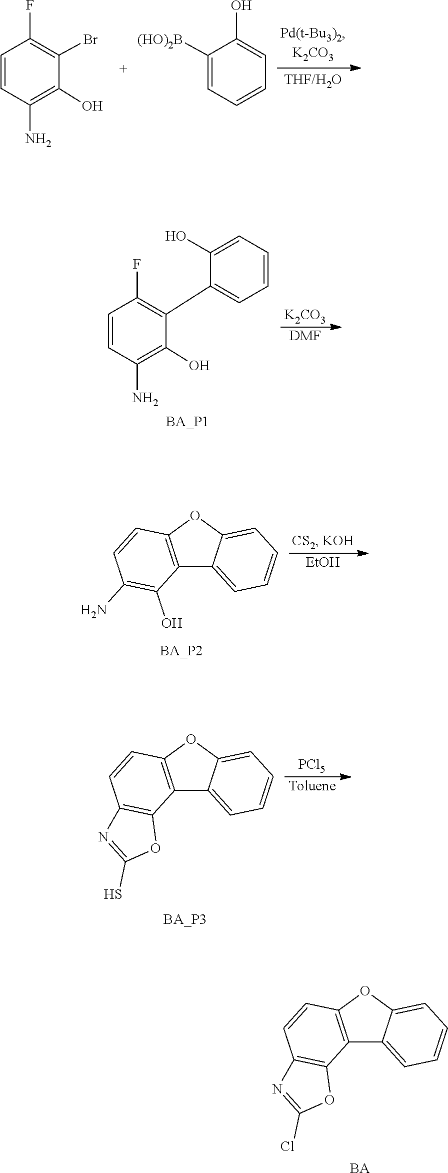

Compound BA was prepared in the same manner as in Preparation Example 1, except that 6-amino-2-bromo-3-fluorophenol was used instead of 2-amino-3-bromo-4-fluorophenol.

### Synthesis Scheme of Preparation Examples 8 and 9

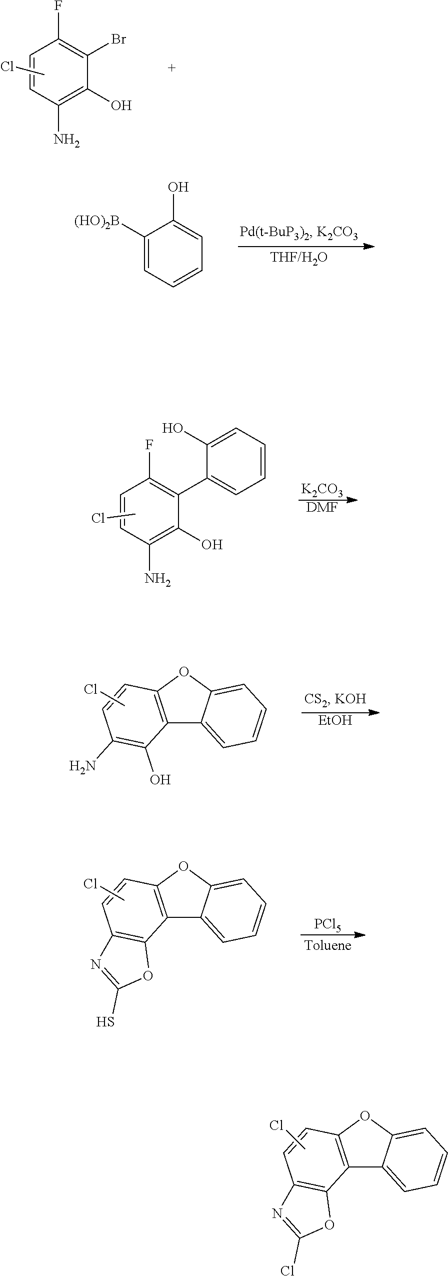

### Preparation Example 8: Preparation of Compound BB

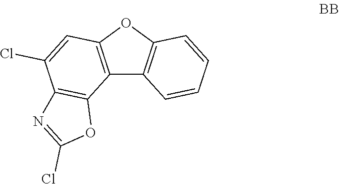 [SMILES: Clc1nc2c(Cl)cc3oc4ccccc4c3c2o1]

Compound BB was prepared in the same manner as in Preparation Example 1, except that 2-amino-6-bromo-3-chloro-5-fluorophenol was used instead of 2-amino-3-bromo-4-fluorophenol.

### Preparation Example 9: Preparation of Compound BC

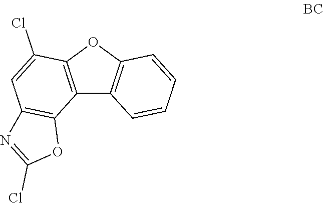 [SMILES: Clc1nc2cc(Cl)c3oc4ccccc4c3c2o1]

Compound BC was prepared in the same manner as in Preparation Example 1, except that 6-amino-2-bromo-4-chloro-3-fluorophenol was used instead of 2-amino-3-bromo-4-fluorophenol.

### Synthesis Scheme of Preparation Examples 10 to 12

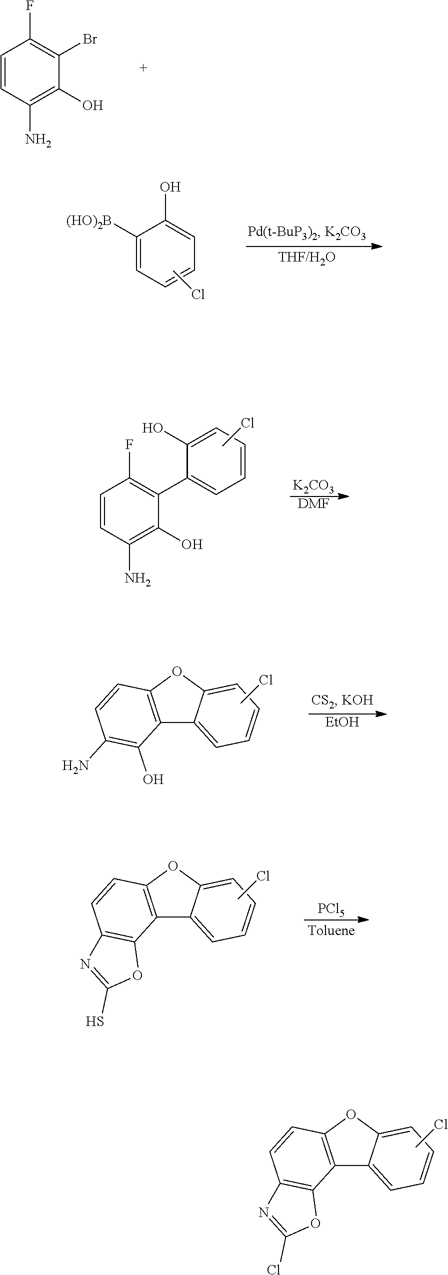

### Preparation Example 10: Preparation of Compound BD

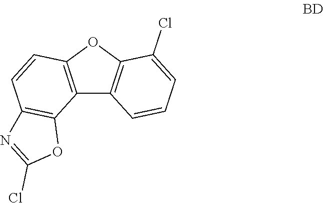 [SMILES: Clc1nc2ccc3oc4c(Cl)cccc4c3c2o1]

Compound BD was prepared in the same manner as in Preparation Example 1, except that 6-amino-2-bromo-3-fluorophenol was used instead of 2-amino-3-bromo-4-fluorophenol, and (3-chloro-2-hydroxyphenyl)boronic acid was used instead of (2-hydroxyphenyl)boronic acid.

### Preparation Example 11: Preparation of Compound BE

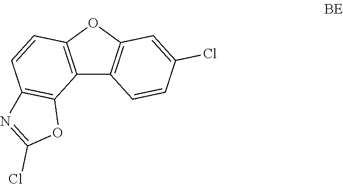 [SMILES: Clc1ccc2c(c1)oc1ccc3nc(Cl)oc3c12]

Compound BE was prepared in the same manner as in Preparation Example 1, except that 6-amino-2-bromo-3-fluorophenol was used instead of 2-amino-3-bromo-4-fluorophenol, and (4-chloro-2-hydroxyphenyl)boronic acid was used instead of (2-hydroxyphenyl)boronic acid.

### Preparation Example 12: Preparation of Compound BF

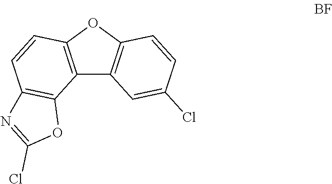 [SMILES: Clc1ccc2oc3ccc4nc(Cl)oc4c3c2c1]

Compound BF was prepared in the same manner as in Preparation Example 1, except that 6-amino-2-bromo-3-fluorophenol was used instead of 2-amino-3-bromo-4-fluorophenol, and (5-chloro-2-hydroxyphenyl)boronic acid was used instead of (2-hydroxyphenyl)boronic acid.

### Preparation Example 13: Preparation of Compound CA

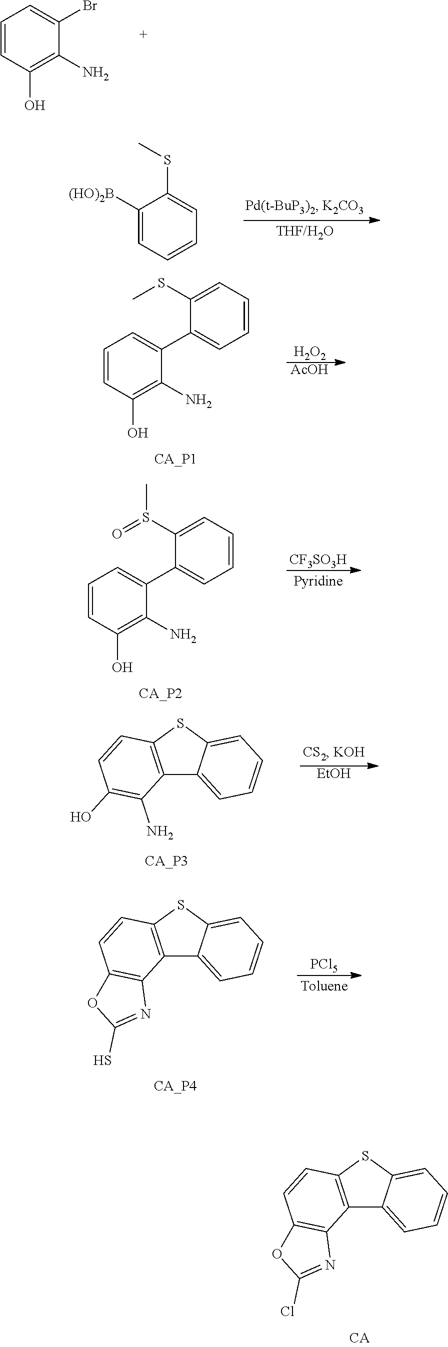

2-Amino-3-bromophenol (15 g, 79.8 mmol) and (2-(methylthio)phenyl)boronic acid (14.1 g, 83.8 mmol) were added to THF (300 ml), and the mixture was stirred and refluxed. Then, potassium carbonate (33.1 g, 239.3 mmol) was dissolved in water (99 ml) and added thereto, and the mixture was sufficiently stirred, and then bis(tri-tert-butylphosphine)palladium(O) (0.4 g, 0.8 mmol) was added. After reacting for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound CA_P1 (11.1 g). (Yield: 60%, MS: [M+H]+=232)

Compound CA_P1 (15 g, 64.9 mmol) and hydrogen peroxide (4.4 g, 129.8 mmol) were added to acetic acid (300 ml), and the mixture was stirred and refluxed. After reacting for 3 hours, the reaction mixture was cooled to room temperature, and then the organic solvent was distilled under reduced pressure. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound CA_P2 (7.7 g). (Yield: 48%, MS: [M+H]+=248)

Compound CA_P2 (15 g, 60.7 mmol) and trifluoromethanesulfonic acid (13.7 g, 91 mmol) were added to pyridine (300 ml), and the mixture was stirred at room temperature. After reacting for 5 hours, the reaction mixture was poured into water, solidified and then filtered. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound CA_P3 (8.1 g). (Yield: 62%, MS: [M+H]+=216)

Compound CA_P3 (15 g, 69.7 mmol) and carbon disulfide (8 g, 104.5 mmol), potassium hydroxide (5.9 g, 104.5 mmol) were added to EtOH (150 ml), and the mixture was stirred and refluxed. After reacting for 5 hours, the reaction mixture was cooled to room temperature, and then the organic solvent was distilled under reduced pressure. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound CA_P4 (12.4 g). (Yield: 69%, MS: [M+H]+=258)

Compound CAP4 (15 g, 58.3 mmol) and Phosphorus pentachloride (12.7 g, 61.2 mmol) were added to Toluene (150 ml), and the mixture was stirred and refluxed. After reacting for 4 hours, the reaction mixture was cooled to room temperature, and then the organic solvent was distilled under reduced pressure. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound CA (9.4 g). (Yield: 62%, MS: [M+H]+=260)

### Synthesis Scheme of Preparation Examples 14 and 15

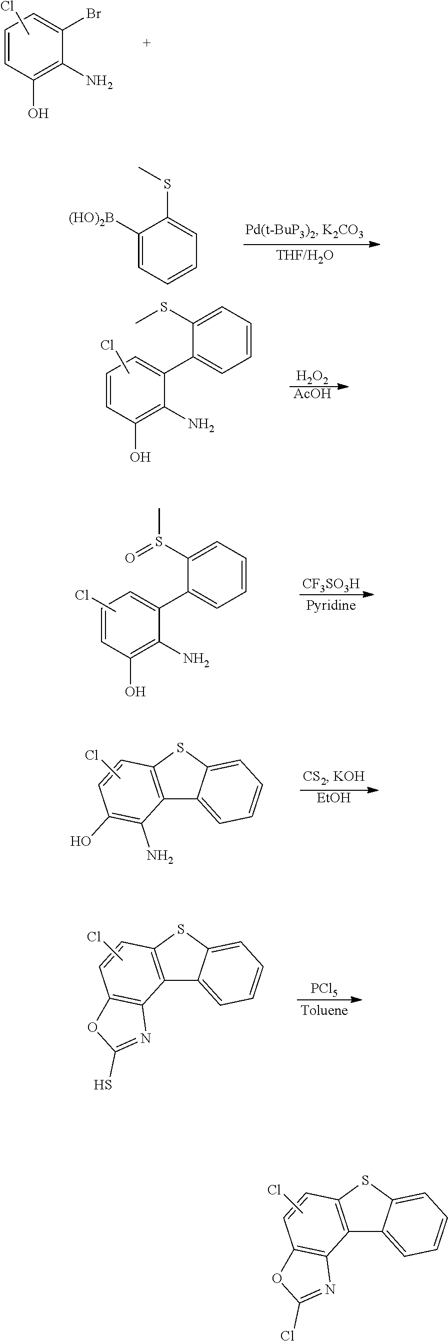

### Preparation Example 14: Preparation of Compound CB

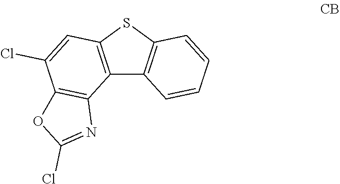 [SMILES: Clc1nc2c(o1)c(Cl)cc1sc3ccccc3c12]

Compound CB was prepared in the same manner as in Preparation Example 13, except that 2-amino-3-bromo-6-chlorophenol was used instead of 2-amino-3-bromophenol.

### Preparation Example 15: Preparation of Compound CC

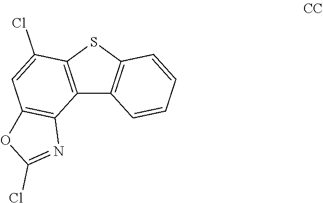 [SMILES: Clc1nc2c(cc(Cl)c3sc4ccccc4c32)o1]

Compound CC was prepared in the same manner as in Preparation Example 13, except that 2-amino-3-bromo-5-chlorophenol was used instead of 2-amino-3-bromophenol.

### Synthesis Scheme of Preparation Examples 16 to 18

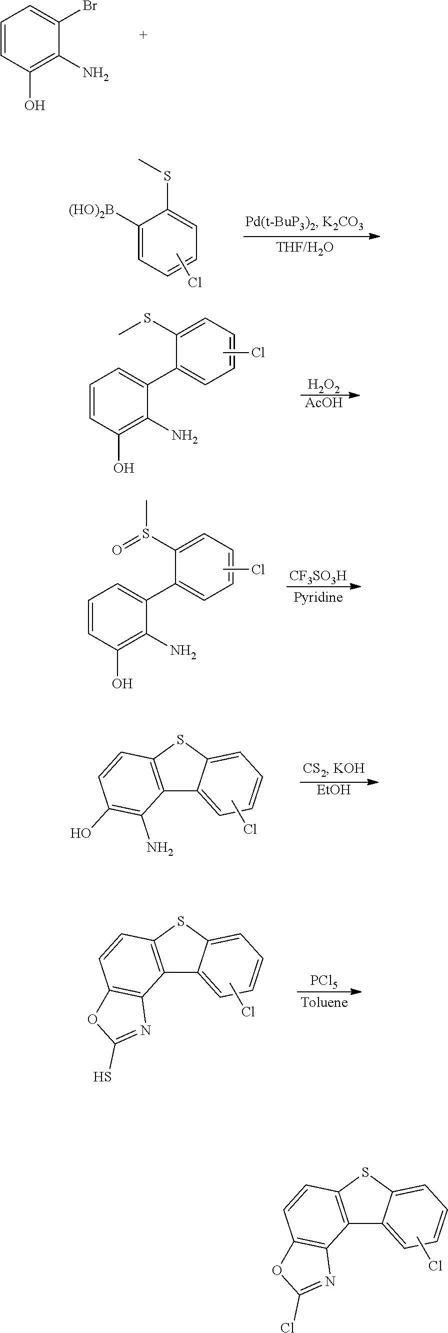

### Preparation Example 16: Preparation of Compound CD

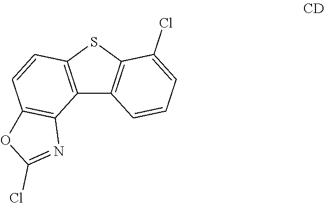 [SMILES: Clc1nc2c(ccc3sc4c(Cl)cccc4c32)o1]

Compound CD was prepared in the same manner as in Preparation Example 13, except that (3-chloro-2-(methylthio)phenyl)boronic acid was used instead of (2-(methylthio)phenyl)boronic acid.

### Preparation Example 17: Preparation of Compound CE

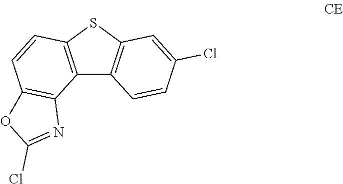 [SMILES: Clc1ccc2c(c1)sc1ccc3oc(Cl)nc3c12]

Compound CE was prepared in the same manner as in Preparation Example 13, except that (4-chloro-2-(methylthio)phenyl)boronic acid was used instead of (2-(methylthio)phenyl)boronic acid.

### Preparation Example 18: Preparation of Compound CF

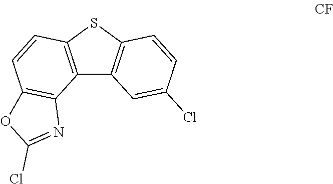 [SMILES: Clc1ccc2sc3ccc4oc(Cl)nc4c3c2c1]

Compound CF was prepared in the same manner as in Preparation Example 13, except that (5-chloro-2-(methylthio)phenyl)boronic acid was used instead of (2-(methylthio)phenyl)boronic acid.

### Preparation Example 19: Preparation of Compound DA

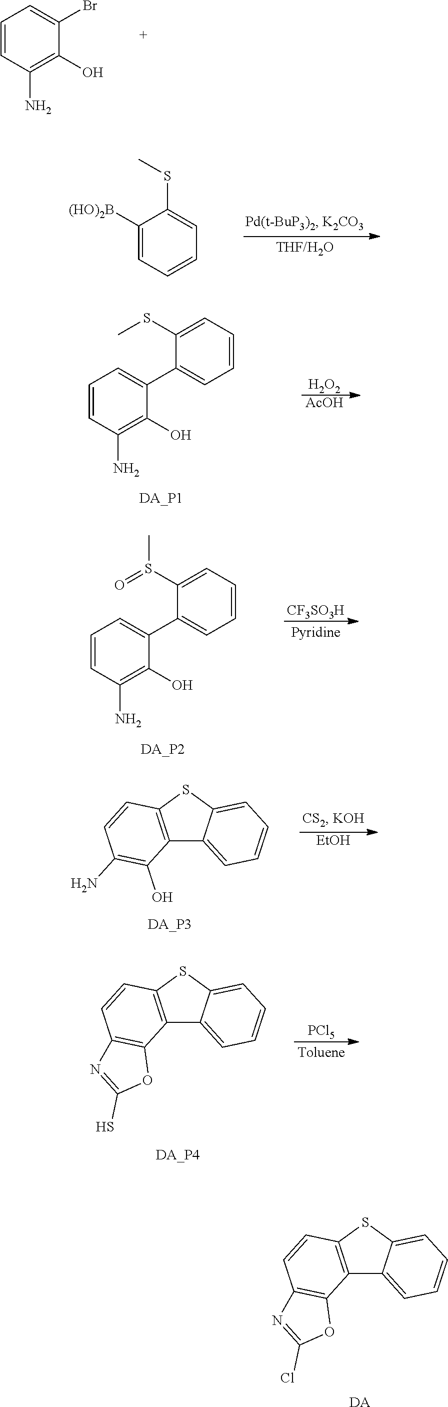

Compound DA was prepared in the same manner as in Preparation Example 13, except that 2-amino-6-bromophenol was used instead of 2-amino-3-bromophenol.

### Synthesis Scheme of Preparation Examples 20 and 21

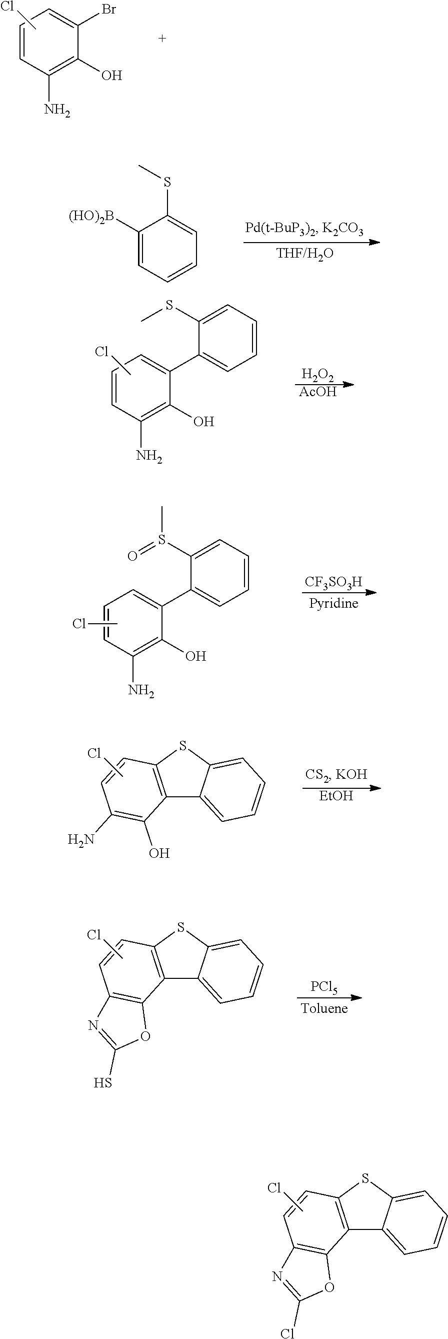

### Preparation Example 20: Preparation of Compound DB

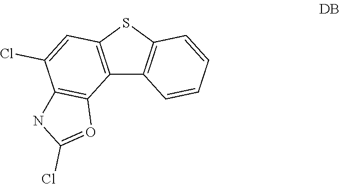

Compound DB was prepared in the same manner as in Preparation Example 13, except that 2-amino-6-bromo-3-chlorophenol was used instead of 2-amino-3-bromophenol.

### Preparation Example 21: Preparation of Compound DC

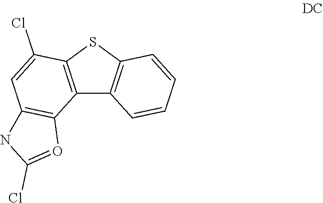

Compound DC was prepared in the same manner as in Preparation Example 13, except that 2-amino-6-bromo-4-chlorophenol was used instead of 2-amino-3-bromophenol.

### Synthesis Scheme of Preparation Examples 22 to 24

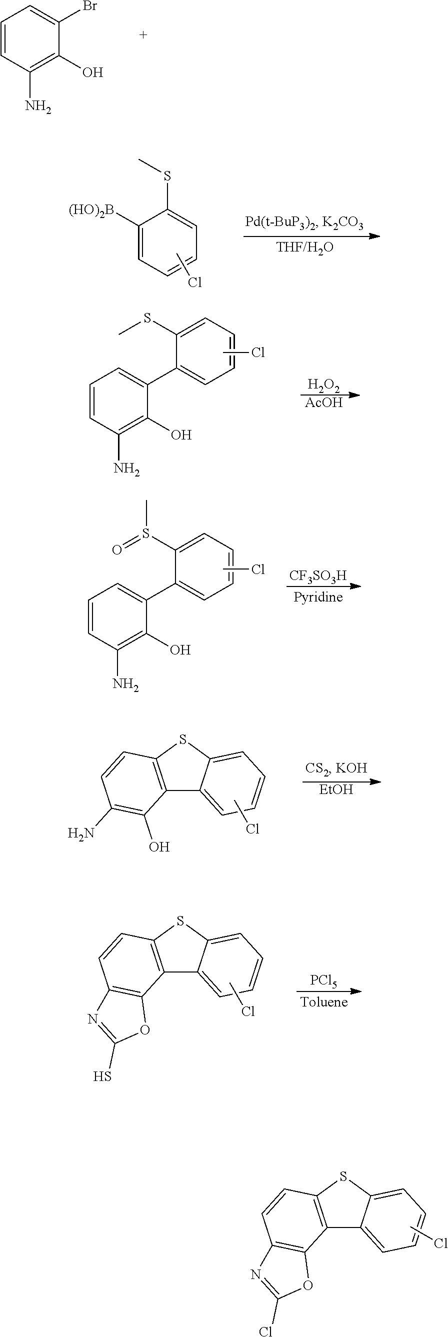

### Preparation Example 22: Preparation of Compound DD

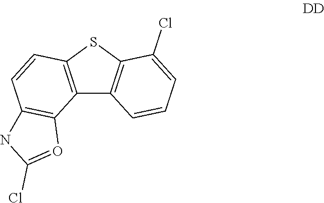

Compound DD was prepared in the same manner as in Preparation Example 13, except that 2-amino-6-bromophenol was used instead of 2-amino-3-bromophenol, and (3-chloro-2-(methylthio)phenyl)boronic acid was used instead of (2-(methylthio)phenyl)boronic acid.

### Preparation Example 23: Preparation of Compound DE

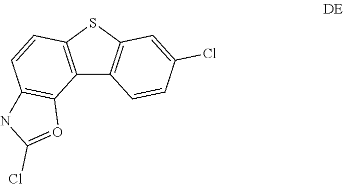

Compound DE was prepared in the same manner as in Preparation Example 13, except that 2-amino-6-bromophenol was used instead of 2-amino-3-bromophenol, and (4-chloro-2-(methylthio)phenyl)boronic acid was used instead of (2-(methylthio)phenyl)boronic acid.

### Preparation Example 24: Preparation of Compound DF

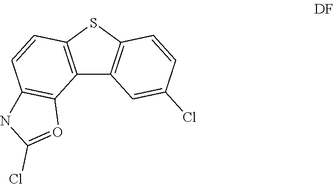

Compound DF was prepared in the same manner as in Preparation Example 13, except that 2-amino-6-bromophenol was used instead of 2-amino-3-bromophenol, and (5-chloro-2-(methylthio)phenyl)boronic acid was used instead of (2-(methylthio)phenyl)boronic acid.

### Preparation Example 25: Preparation of Compound EA

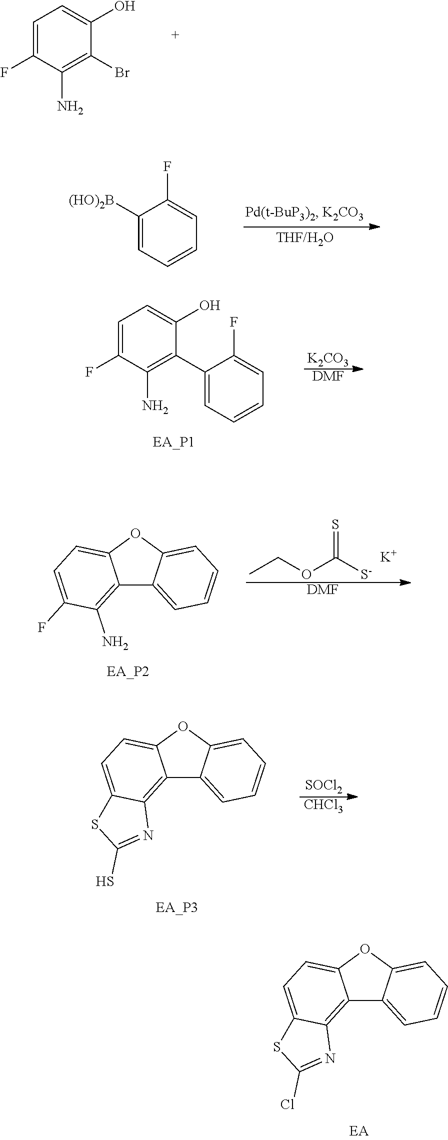

3-Amino-2-bromo-4-fluorophenol (15 g, 72.8 mmol) and (2-fluorophenyl)boronic acid (10.7 g, 76.5 mmol) were added to THF (300 ml), and the mixture was stirred and refluxed. Then, potassium carbonate (30.2 g, 218.4 mmol) was dissolved in water (91 ml) and added thereto, and the mixture was sufficiently stirred, and then bis(tri-tert-butylphosphine) palladium (0) (0.4 g, 0.7 mmol) was added. After reacting for 2 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound EA_P1 (10.8 g). (Yield: 67%, MS: [M+H]+=222)

Compound EA_P1 (15 g, 67.8 mmol) and potassium carbonate (28.1 g, 203.4 mmol) were added to DMF (150 ml), and the mixture was stirred and refluxed. After reacting for 4 hours, the reaction mixture was cooled to room temperature, and then the organic solvent was distilled under reduced pressure. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound EA_P2 (9.7 g). (Yield: 71%, MS: [M+H]+=202)

Compound EA_P2 (15 g, 74.6 mmol) and potassium O-ethyl dithiocarbonate (29.9 g, 186.4 mmol) were added to DMF (150 ml), and the mixture was stirred and refluxed. After reacting for 9 hours, the reaction mixture was cooled to room temperature, and then the organic solvent was distilled under reduced pressure. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound EA_P3 (11.5 g). (Yield: 60%, MS: [M+H]+=258)

Compound EA_P3 (15 g, 58.3 mmol) was added to CHCl₃ (150 ml), and cooled up to 0° C. Thionyl chloride (17.3 g, 145.7 mmol) was added dropwise thereto, and then stirred. After reacting for 4 hours, the reaction mixture was cooled to room temperature, and then the organic solvent was distilled under reduced pressure. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound EA (8 g). (Yield: 53%, MS: [M+H]+=260)

### Synthesis Scheme of Preparation Examples 26 and 27

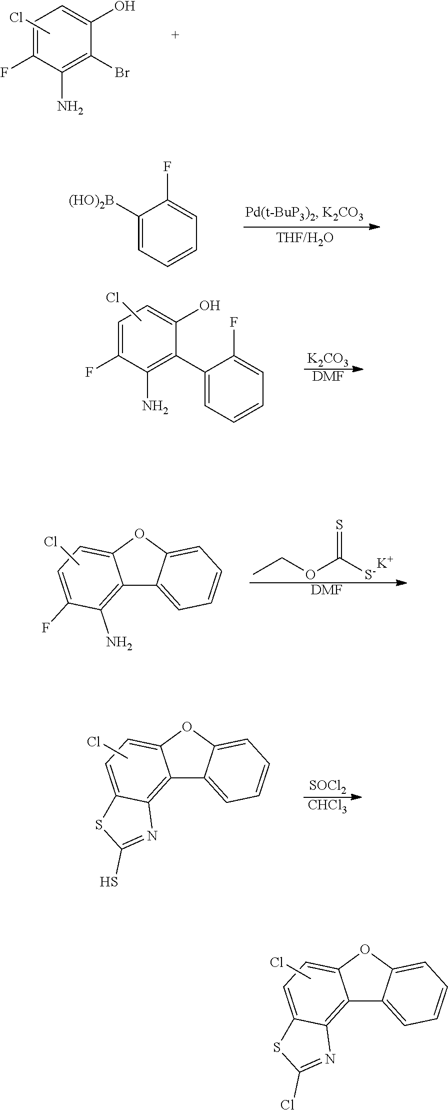

### Preparation Example 26: Preparation of Compound EB

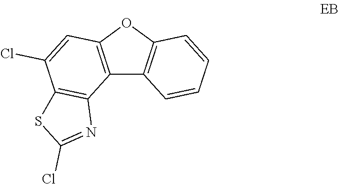 [SMILES: Clc1nc2c(s1)c(Cl)cc1oc3ccccc3c12]

Compound EB was prepared in the same manner as in Preparation Example 25, except that 3-amino-2-bromo-5-chloro-4-fluorophenol was used instead of 3-amino-2-bromo-4-fluorophenol.

### Preparation Example 27: Preparation of Compound EC

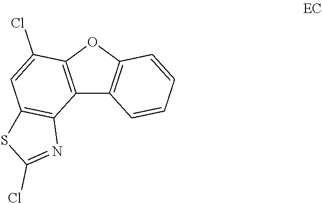 [SMILES: Clc1nc2c(cc(Cl)c3oc4ccccc4c32)s1]

Compound EC was prepared in the same manner as in Preparation Example 25, except that 3-amino-2-bromo-6-chloro-4-fluorophenol was used instead of 3-amino-2-bromo-4-fluorophenol.

### Synthesis Scheme of Preparation Examples 28 to 30

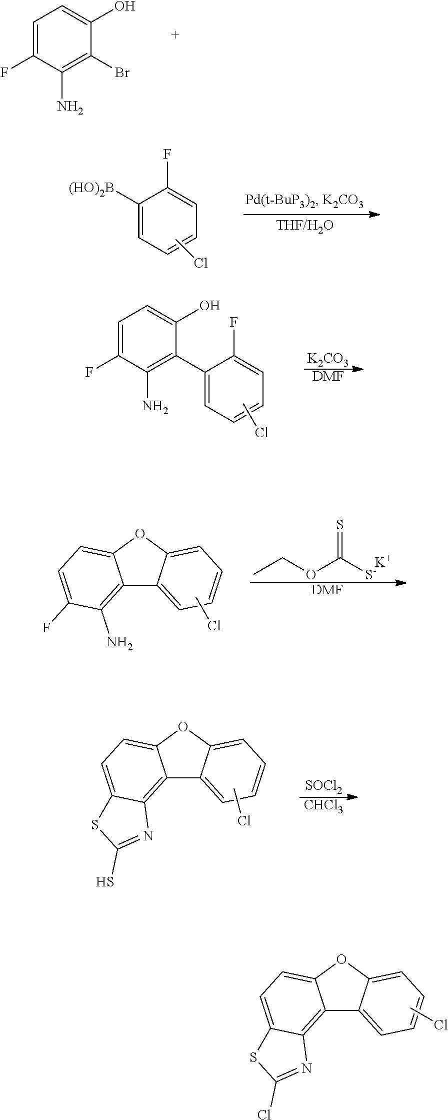

### Preparation Example 28: Preparation of Compound ED

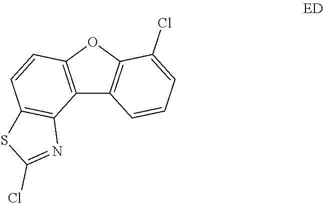 [SMILES: Clc1nc2c(ccc3oc4c(Cl)cccc4c32)s1]

Compound ED was prepared in the same manner as in Preparation Example 25, except that (3-chloro-2-fluorophenyl)boronic acid was used instead of (2-fluorophenyl)boronic acid.

### Preparation Example 29: Preparation of Compound EE

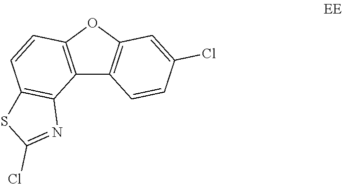 [SMILES: Clc1ccc2c(c1)oc1ccc3sc(Cl)nc3c12]

Compound EE was prepared in the same manner as in Preparation Example 25, except that (4-chloro-2-fluorophenyl)boronic acid was used instead of (2-fluorophenyl)boronic acid.

### Preparation Example 30: Preparation of Compound EF

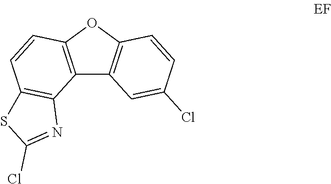 [SMILES: Clc1ccc2oc3ccc4sc(Cl)nc4c3c2c1]

Compound EF was prepared in the same manner as in Preparation Example 25, except that (5-chloro-2-fluorophenyl)boronic acid was used instead of (2-fluorophenyl)boronic acid.

### Preparation Example 31: Preparation of Compound FA

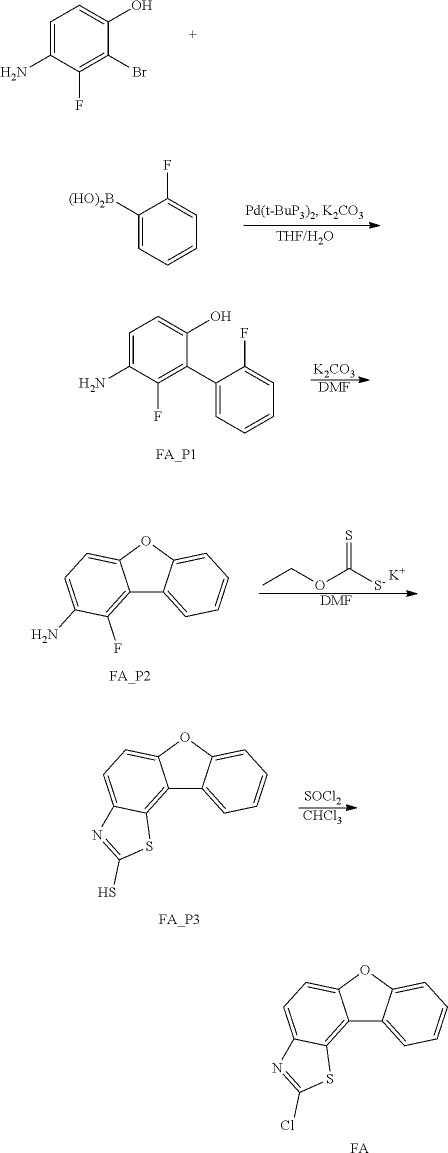

Compound FA was prepared in the same manner as in Preparation Example 25, except that 4-amino-2-bromo-3-fluorophenol was used instead of 3-amino-2-bromo-4-fluorophenol.

### Synthesis Scheme of Preparation Examples 32 and 33

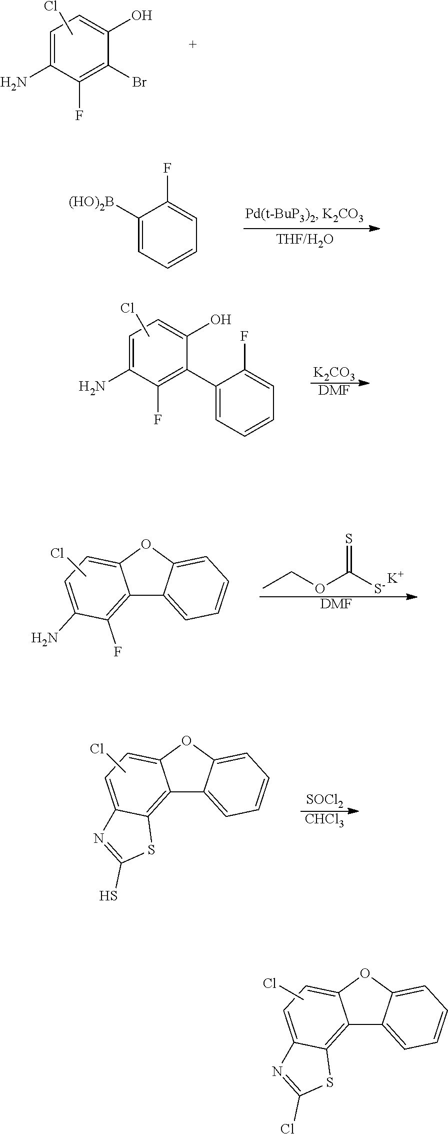

### Preparation Example 32: Preparation of Compound FB

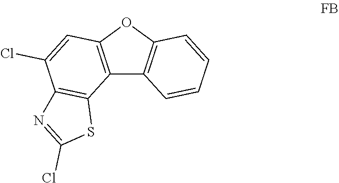 [SMILES: Clc1nc2c(Cl)cc3oc4ccccc4c3c2s1]

Compound FB was prepared in the same manner as in Preparation Example 25, except that 4-amino-2-bromo-5-chloro-3-fluorophenol was used instead of 3-amino-2-bromo-4-fluorophenol.

### Preparation Example 33: Preparation of Compound FC

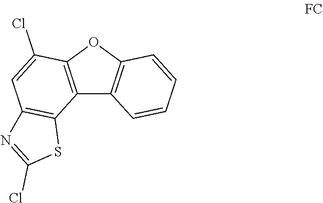 [SMILES: Clc1nc2cc(Cl)c3oc4ccccc4c3c2s1]

Compound FC was prepared in the same manner as in Preparation Example 25, except that 4-amino-2-bromo-6-chloro-3-fluorophenol was used instead of 3-amino-2-bromo-4-fluorophenol.

### Synthesis Scheme of Preparation Examples 34 to 36

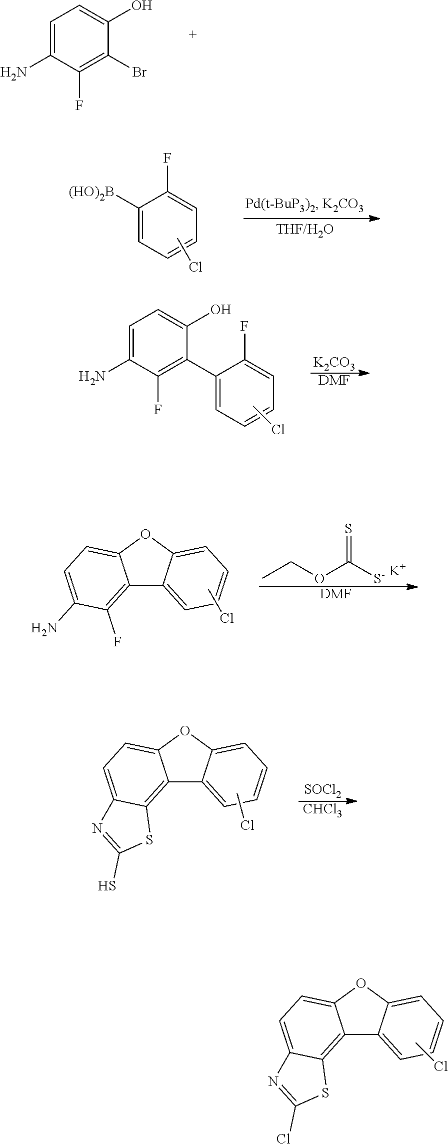

### Preparation Example 34: Preparation of Compound FD

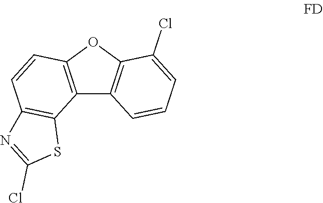 [SMILES: Clc1nc2ccc3oc4c(Cl)cccc4c3c2s1]

Compound FD was prepared in the same manner as in Preparation Example 25, except that 4-amino-2-bromo-3-fluorophenol was used instead of 3-amino-2-bromo-4-fluorophenol, and (3-chloro-2-fluorophenyl)boronic acid was used instead of (2-fluorophenyl)boronic acid.

### Preparation Example 35: Preparation of Compound FE

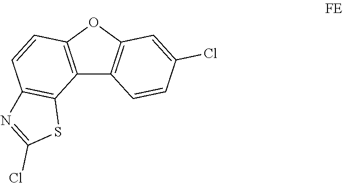 [SMILES: Clc1ccc2c(c1)oc1ccc3nc(Cl)sc3c12]

Compound FE was prepared in the same manner as in Preparation Example 25, except that 4-amino-2-bromo-3-fluorophenol was used instead of 3-amino-2-bromo-4-fluorophenol, and (4-chloro-2-fluorophenyl)boronic acid was used instead of (2-fluorophenyl)boronic acid.

### Preparation Example 36: Preparation of Compound FF

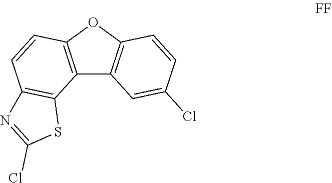 [SMILES: Clc1ccc2oc3ccc4nc(Cl)sc4c3c2c1]

Compound FF was prepared in the same manner as in Preparation Example 25, except that 4-amino-2-bromo-3-fluorophenol was used instead of 3-amino-2-bromo-4-fluorophenol, and (5-chloro-2-fluorophenyl)boronic acid was used instead of (2-fluorophenyl)boronic acid.

### Preparation Example 37: Preparation of Compound GA

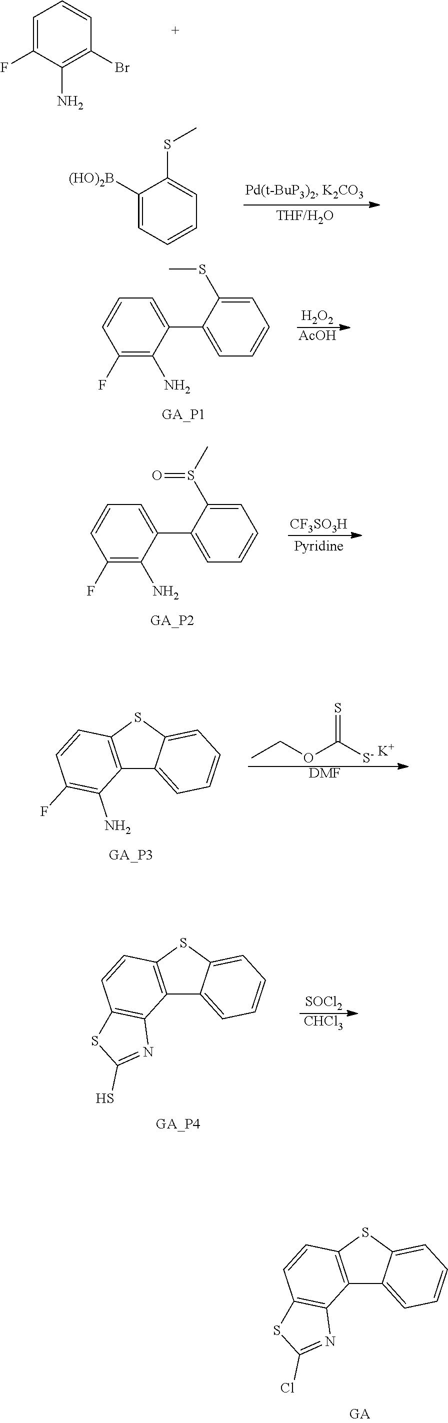

2-Bromo-6-fluoroaniline (15 g, 78.9 mmol) and (2-(methylthio)phenyl)boronic acid (13.9 g, 82.9 mmol) were added to THF (300 ml), and the mixture was stirred and refluxed. Then, potassium carbonate (32.7 g, 236.8 mmol) was dissolved in water (98 ml) and added thereto, and the mixture was sufficiently stirred, and then bis(tri-tert-butylphosphine)palladium(0) (0.4 g, 0.8 mmol) was added. After reacting for 2 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound GA_P1 (11 g). (Yield: 60%, MS: [M+H]+=234)

Compound GA_P1 (15 g, 64.3 mmol) and hydrogen peroxide (4.4 g, 128.6 mmol) were added to acetic acid (300 ml), and the mixture was stirred and refluxed. After reacting for 4 hours, the reaction mixture was cooled to room temperature, and then the organic solvent was distilled under reduced pressure. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound GA_P2 (7.5 g). (Yield: 47%, MS: [M+H]+=250)

Compound GA_P2 (15 g, 60.2 mmol) and trifluoromethanesulfonic acid (13.5 g, 90.3 mmol) were added to pyridine (300 ml), and stirred at room temperature. After reacting for 11 hours, the reaction mixture was poured into water (600 ml), solidified and then filtered. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound GA_P3 (9 g). (Yield: 69%, MS: [M+H]+=218)

Compound GA_P3 (15 g, 69 mmol) and potassium O-ethyl dithiocarbonate (27.7 g, 172.6 mmol) were added to DMF (150 ml), and the mixture was stirred and refluxed. After reacting for 9 hours, the reaction mixture was cooled to room temperature, and then the organic solvent was distilled under reduced pressure. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound GA_P4 (11.9 g). (Yield: 63%, MS: [M+H]+=274)

Compound GA_P4 (15 g, 54.9 mmol) was added to CHCl₃ (150 ml) and cooled up to 0° C. Thionyl chloride (16.3 g, 137.2 mmol) was added dropwise thereto and stirred. After reacting for 4 hours, the reaction mixture was cooled to room temperature, and then the organic solvent was distilled under reduced pressure. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound GA (7.2 g). (Yield: 48%, MS: [M+H]+=276)

### Synthesis Scheme of Preparation Examples 38 and 39

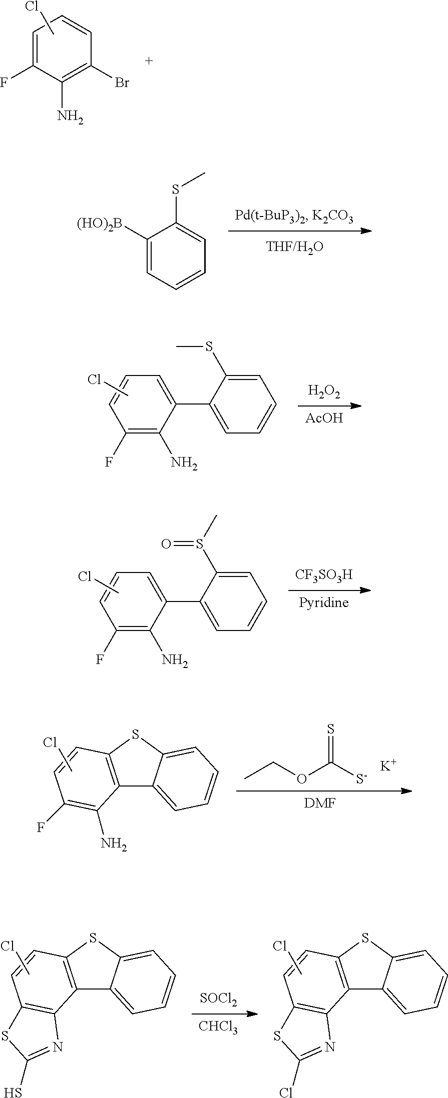

### Preparation Example 38: Preparation of Compound GB

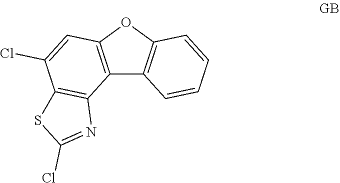 [SMILES: Clc1nc2c(s1)c(Cl)cc1oc3ccccc3c12]

Compound GB was prepared in the same manner as in Preparation Example 37, except that 6-bromo-3-chloro-2-fluoroaniline was used instead of 2-bromo-6-fluoroaniline.

### Preparation Example 39: Preparation of Compound GC

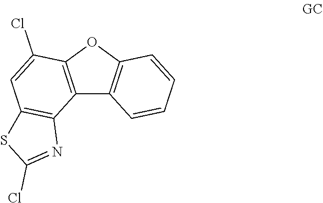 [SMILES: Clc1nc2c(cc(Cl)c3oc4ccccc4c32)s1]

Compound GC was prepared in the same manner as in Preparation Example 37, except that 2-bromo-4-chloro-6-fluoroaniline was used instead of 2-bromo-6-fluoroaniline.

### Synthesis Scheme of Preparation Examples 40 to 42

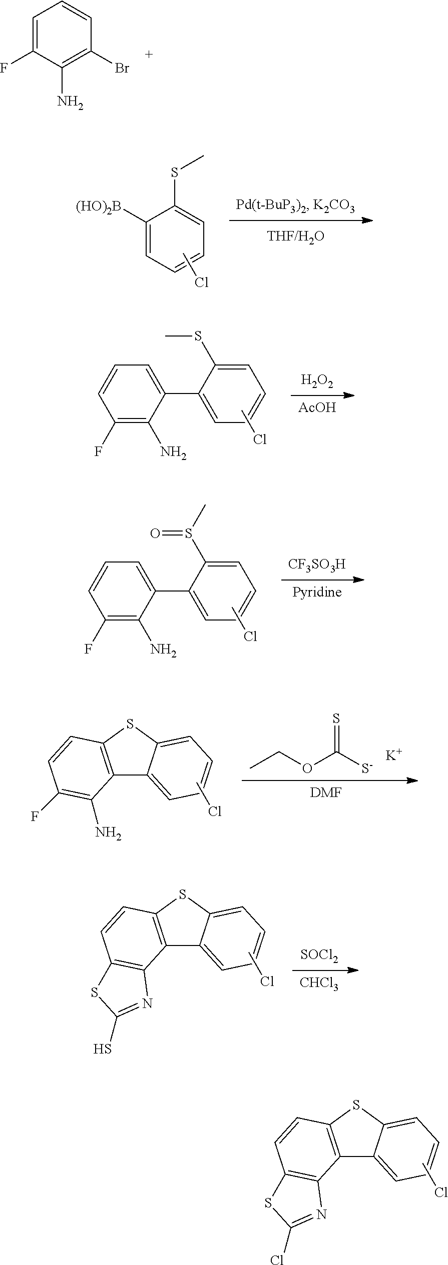

### Preparation Example 40: Preparation of Compound GD

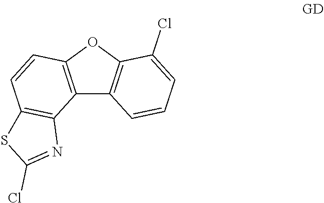 [SMILES: Clc1nc2c(ccc3oc4c(Cl)cccc4c32)s1]

Compound GD was prepared in the same manner as in Preparation Example 37, except that (3-chloro-2-(methylthio)phenyl)boronic acid was used instead of (2-(methylthio)phenyl)boronic acid.

### Preparation Example 41: Preparation of Compound GE

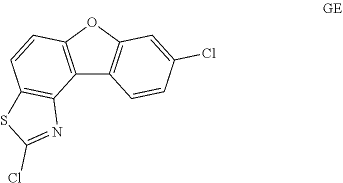 [SMILES: Clc1ccc2c(c1)oc1ccc3sc(Cl)nc3c12]

Compound GE was prepared in the same manner as in Preparation Example 37, except that (4-chloro-2-(methylthio)phenyl)boronic acid was used instead of (2-(methylthio)phenyl)boronic acid.

### Preparation Example 42: Preparation of Compound GF

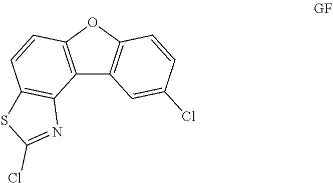 [SMILES: Clc1ccc2oc3ccc4sc(Cl)nc4c3c2c1]

Compound GF was prepared in the same manner as in Preparation Example 37, except that (5-chloro-2-(methylthio)phenyl)boronic acid was used instead of (2-(methylthio)phenyl)boronic acid.

### Preparation Example 43: Preparation of Compound HA

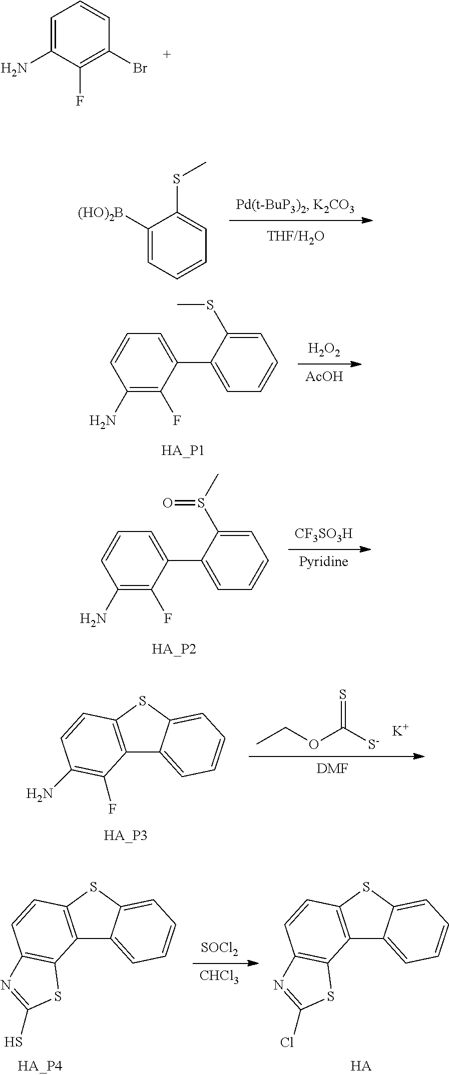

Compound HA was prepared in the same manner as in Preparation Example 37, except that 3-bromo-2-fluoroaniline was used instead of 2-bromo-6-fluoroaniline.

### Synthesis Scheme of Preparation Examples 44 and 45

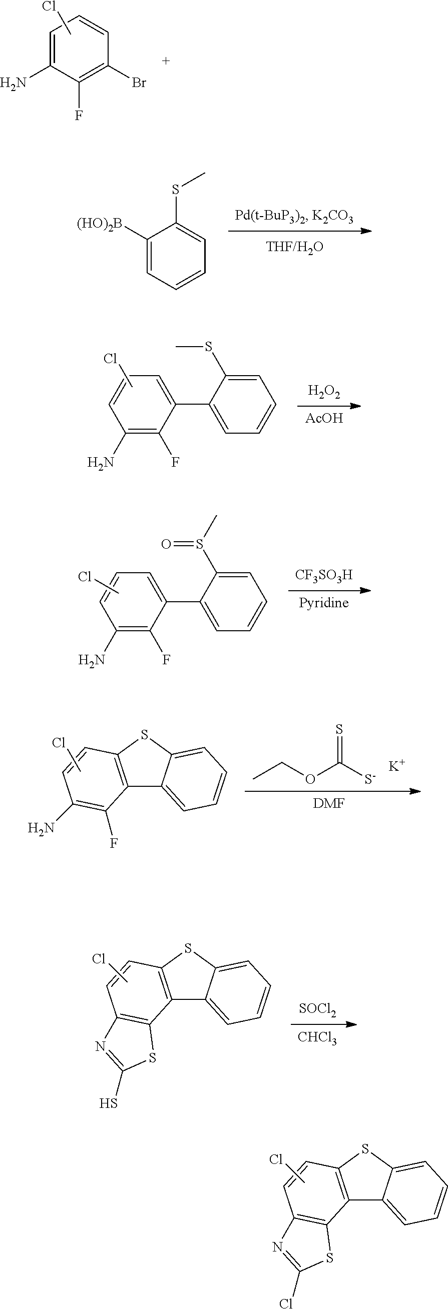

### Preparation Example 44: Preparation of Compound HB

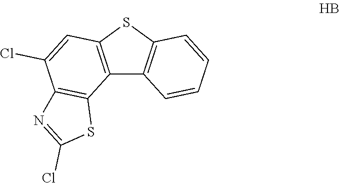 [SMILES: Clc1nc2c(Cl)cc3sc4ccccc4c3c2s1]

Compound HB was prepared in the same manner as in Preparation Example 37, except that 3-bromo-6-chloro-2-fluoroaniline was used instead of 2-bromo-6-fluoroaniline.

### Preparation Example 45: Preparation of Compound HC

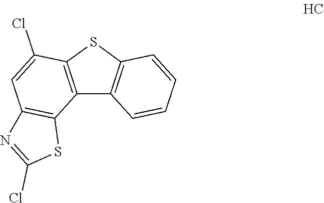 [SMILES: Clc1nc2cc(Cl)c3sc4ccccc4c3c2s1]

Compound HC was prepared in the same manner as in Preparation Example 37, except that 3-bromo-5-chloro-2-fluoroaniline was used instead of 2-bromo-6-fluoroaniline.

### Synthesis Scheme of Preparation Examples 46 to 48

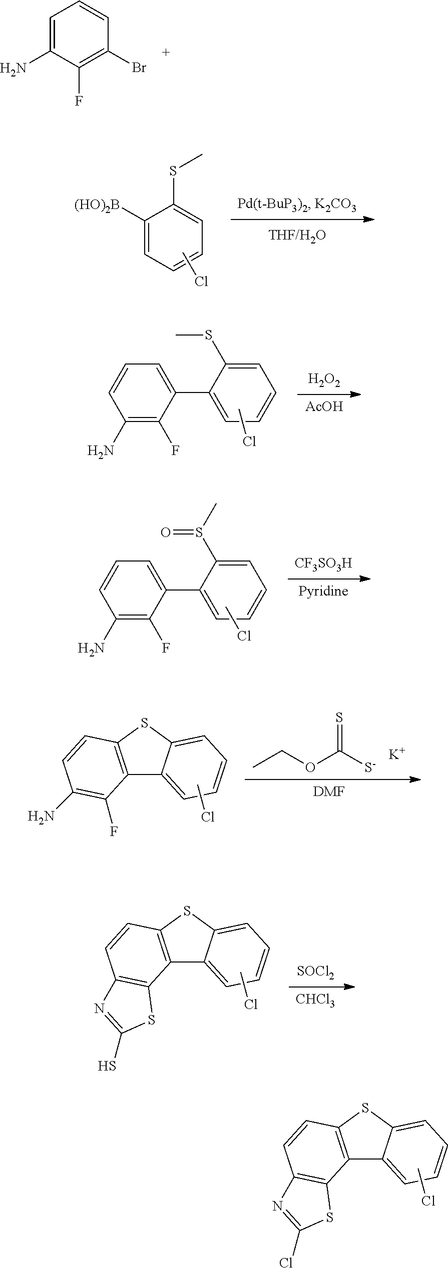

### Preparation Example 46: Preparation of Compound HD

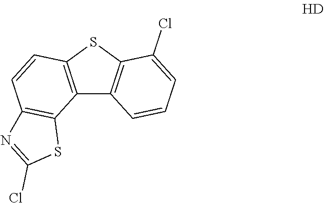 [SMILES: Clc1nc2ccc3sc4c(Cl)cccc4c3c2s1]

Compound HD was prepared in the same manner as in Preparation Example 37, except that 3-bromo-2-fluoroaniline was used instead of 2-bromo-6-fluoroaniline, and (3-chloro-2-(methylthio)phenyl)boronic acid was used instead of (2-(methylthio)phenyl)boronic acid.

### Preparation Example 47: Preparation of Compound HE

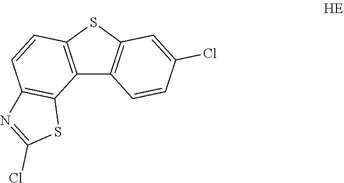 [SMILES: Clc1ccc2c(c1)sc1ccc3nc(Cl)sc3c12]

Compound HE was prepared in the same manner as in Preparation Example 37, except that 3-bromo-2-fluoroaniline was used instead of 2-bromo-6-fluoroaniline, and (4-chloro-2-(methylthio)phenyl)boronic acid was used instead of (2-(methylthio)phenyl)boronic acid.

### Preparation Example 48: Preparation of Compound HF

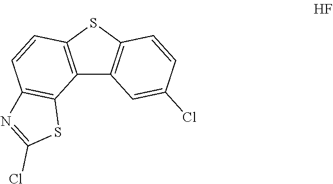 [SMILES: Clc1ccc2sc3ccc4nc(Cl)sc4c3c2c1]

Compound HF was prepared in the same manner as in Preparation Example 37, except that 3-bromo-2-fluoroaniline was used instead of 2-bromo-6-fluoroaniline, and (5-chloro-2-(methylthio)phenyl)boronic acid was used instead of (2-(methylthio)phenyl)boronic acid.

### Example 1: Preparation of Compound 1

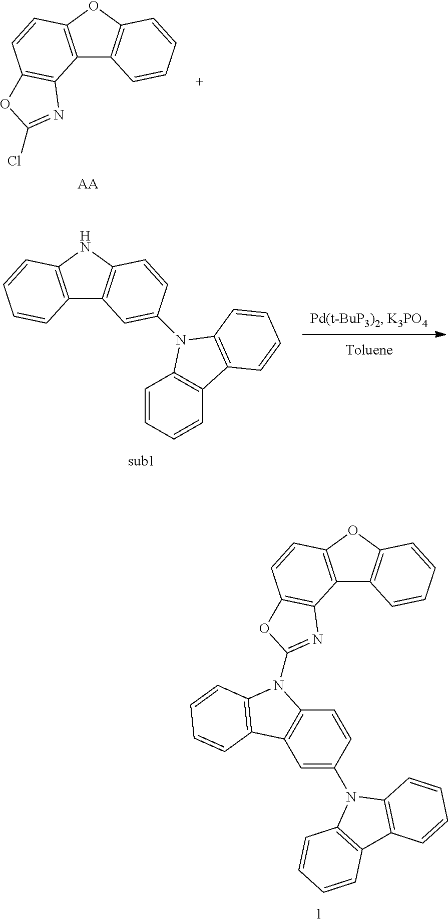 [SMILES: Clc1nc2c(ccc3oc4ccccc4c32)o1 ; "AA" ; c1ccc2c(c1)[nH]c1ccc(-n3c4ccccc4c4ccccc43)cc12 ; c1ccc2c(c1)oc1ccc3oc(-n4c5ccccc5c5cc(-n6c7ccccc7c7ccccc76)ccc54)nc3c12]

Compound AA (10 g, 41 mmol), sub1 (14.3 g, 43.1 mmol) and potassium phosphate (26.1 g, 123.1 mmol) were added to toluene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound 1 (13.7 g). (Yield: 62%, MS: [M+H]+=540)

### Example 2: Preparation of Compound 2

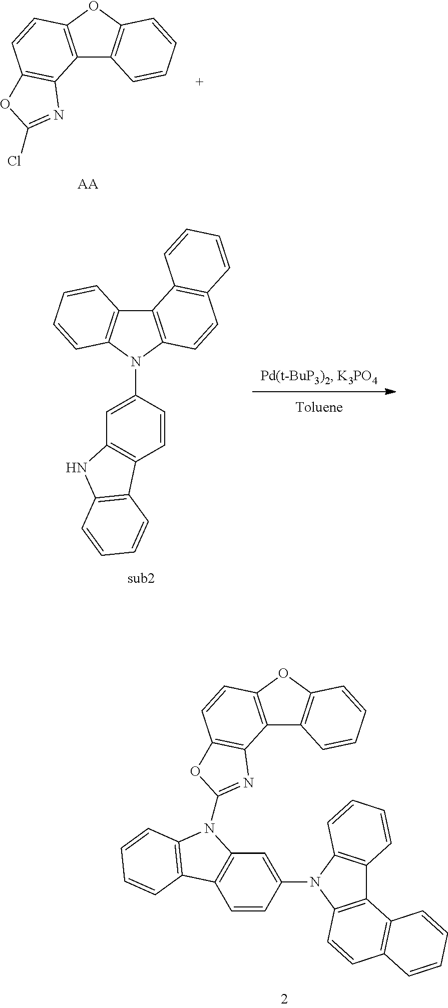 [SMILES: Clc1nc2c(ccc3oc4ccccc4c32)o1 ; "AA" ; c1ccc2c(c1)ccc1c2c2ccccc2n1-c1ccc2c(c1)[nH]c1ccccc12 ; c1ccc2c(c1)ccc1c2c2ccccc2n1-c1ccc2c3ccccc3n(-c3nc4c(ccc5oc6ccccc6c54)o3)c2c1]

Compound AA (10 g, 41 mmol), sub2 (16.5 g, 43.1 mmol) and potassium phosphate (26.1 g, 1231 mmol) were added to toluene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound 2 (14.8 g). (Yield: 61%, MS: [M+H]+=590)

### Example 3: Preparation of Compound 3

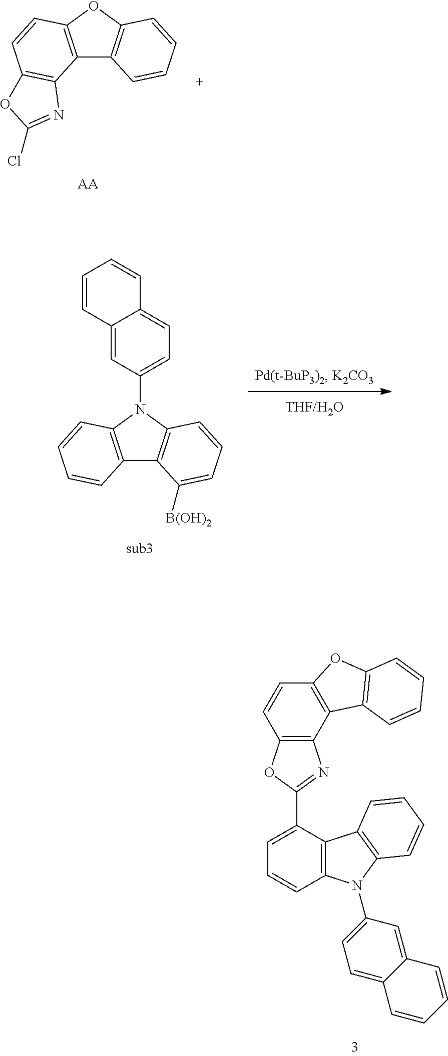 [SMILES: Clc1nc2c(ccc3oc4ccccc4c32)o1 ; "AA" ; OB(O)c1cccc2c1c1ccccc1n2-c1ccc2ccccc2c1 ; c1ccc2cc(-n3c4ccccc4c4c(-c5nc6c(ccc7oc8ccccc8c76)o5)cccc43)ccc2c1]

Compound AA (15 g, 61.6 mmol) and sub3 (21.8 g, 64.6 mmol) were added to THF (300 ml), and the mixture was stirred and refluxed. Then, potassium carbonate (25.5 g, 184.7 mmol) was dissolved in water (77 ml) and added thereto, and the mixture was sufficiently stirred, and then bis(tri-tert-butylphosphine)palladium(0) (0.3 g, 0.6 mmol) was added. After reacting for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 3 (20.3 g). (Yield: 60%, MS: [M+H]+=551)

### Example 4: Preparation of Compound 4

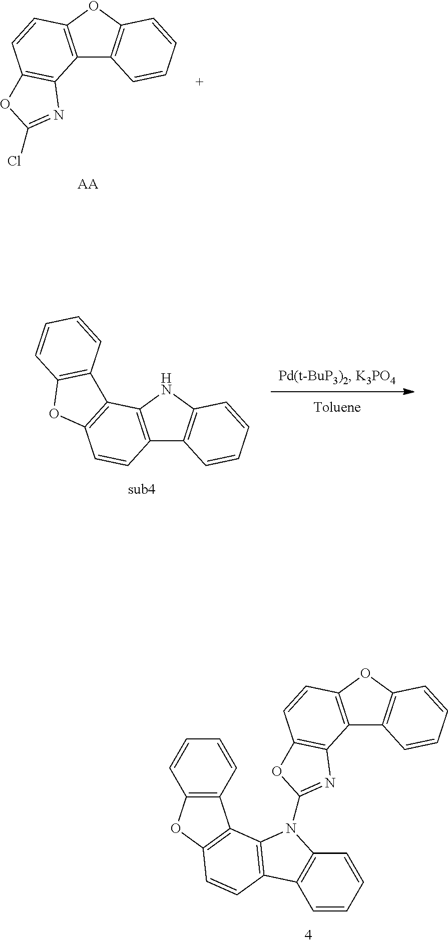 [SMILES: Clc1nc2c(ccc3oc4ccccc4c32)o1 ; "AA" ; c1ccc2c(c1)[nH]c1c2ccc2oc3ccccc3c21 ; c1ccc2c(c1)oc1ccc3oc(-n4c5ccccc5c5ccc6oc7ccccc7c6c54)nc3c12]

Compound AA (10 g, 41 mmol), sub4 (11.1 g, 43.1 mmol) and potassium phosphate (26.1 g, 123.1 mmol) were added to toluene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound 4 (13.1 g). (Yield: 69%, MS: [M+H]+=465)

### Example 5: Preparation of Compound 5

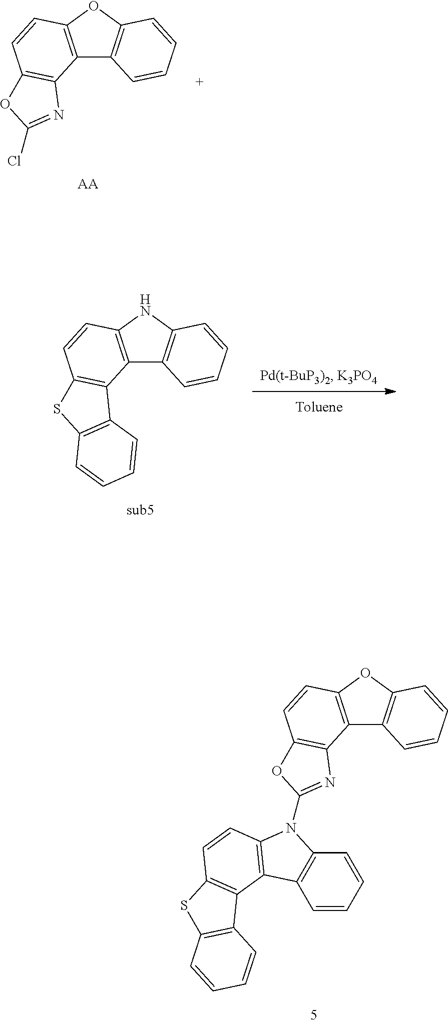 [SMILES: Clc1nc2c(ccc3oc4ccccc4c32)o1 ; "AA" ; c1ccc2c(c1)[nH]c1ccc3sc4ccccc4c3c12 ; c1ccc2c(c1)oc1ccc3oc(-n4c5ccccc5c5c6c(ccc54)sc4ccccc46)nc3c12]

Compound AA (10 g, 41 mmol), sub5 (11.8 g, 43.1 mmol) and potassium phosphate (26.1 g, 1231 mmol) were added to toluene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound 5 (12.8 g). (Yield: 65%, MS: [M+H]+=481)

### Example 6: Preparation of Compound 6

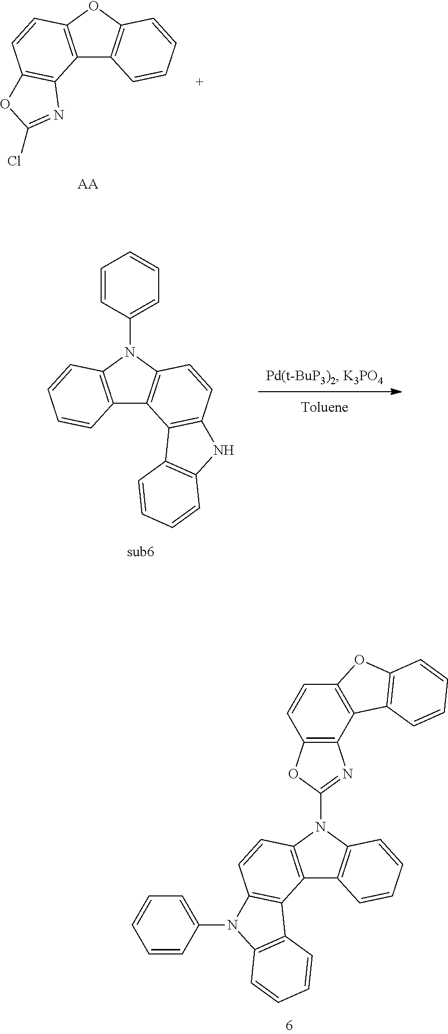 [SMILES: Clc1nc2c(ccc3oc4ccccc4c32)o1 ; "AA" ; c1ccc(-n2c3ccccc3c3c4c(ccc32)[nH]c2ccccc24)cc1 ; c1ccc(-n2c3ccccc3c3c4c5ccccc5n(-c5nc6c(ccc7oc8ccccc8c76)o5)c4ccc32)cc1]

Compound AA (10 g, 41 mmol), sub6 (14.3 g, 43.1 mmol) and potassium phosphate (26.1 g, 123.1 mmol) were added to toluene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound 6 (14.8 g). (Yield: 67%, MS: [M+H]+=540)

### Example 7: Preparation of Compound 7

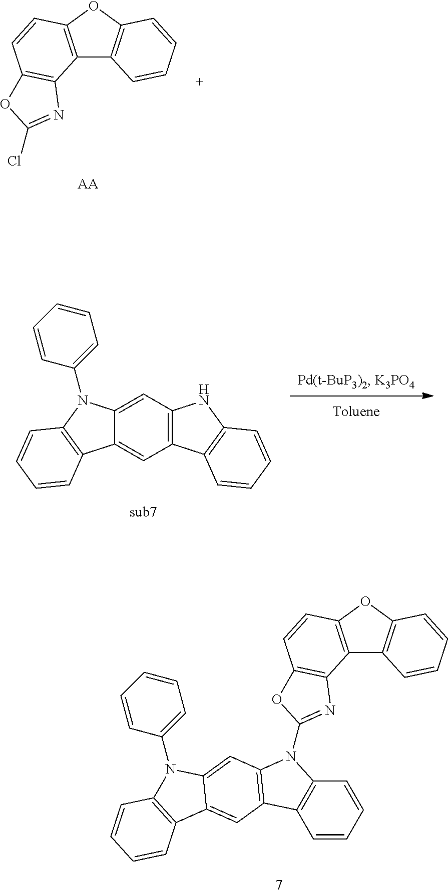 [SMILES: Clc1nc2c(ccc3oc4ccccc4c32)o1 ; "AA" ; c1ccc(-n2c3ccccc3c3cc4c(cc32)[nH]c2ccccc24)cc1 ; c1ccc(-n2c3ccccc3c3cc4c5ccccc5n(-c5nc6c(ccc7oc8ccccc8c76)o5)c4cc32)cc1]

Compound AA (10 g, 41 mmol), sub7 (14.3 g, 43.1 mmol) and potassium phosphate (26.1 g, 123.1 mmol) were added to toluene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound 7 (13.3 g). (Yield: 60%, MS: [M+H]+=540)

### Example 8: Preparation of Compound 8

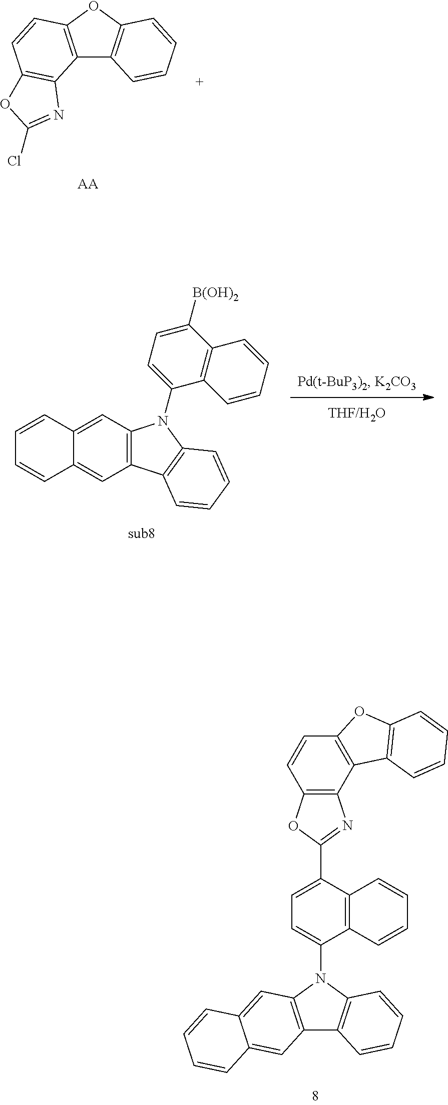 [SMILES: Clc1nc2c(ccc3oc4ccccc4c32)o1 ; "AA" ; OB(O)c1ccc(-n2c3ccccc3c3cc4ccccc4cc32)c2ccccc12 ; c1ccc2cc3c(cc2c1)c1ccccc1n3-c1ccc(-c2nc3c(ccc4oc5ccccc5c43)o2)c2ccccc12]

Compound AA (15 g, 61.6 mmol) and sub8 (25 g, 64.6 mmol) were added to THF (300 ml), and the mixture was stirred and refluxed. Then, potassium carbonate (25.5 g, 184.7 mmol) was dissolved in water (77 ml) and added thereto, and the mixture was sufficiently stirred, and then bis(tri-tert-butylphosphine)palladium(0) (0.3 g, 0.6 mmol) was added. After reacting for 2 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 8 (24.4 g). (Yield: 72%, MS: [M+H]+=551)

### Example 9: Preparation of Compound 9

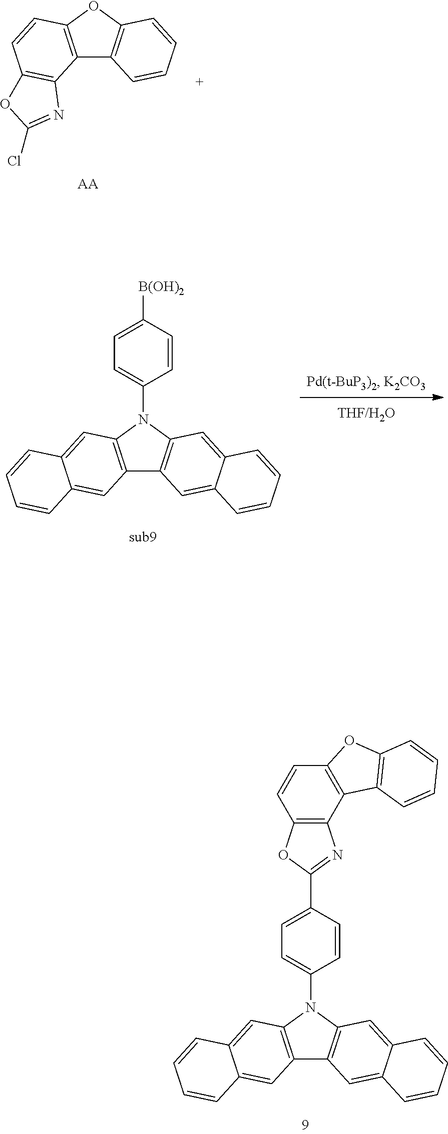 [SMILES: Clc1nc2c(ccc3oc4ccccc4c32)o1 ; "AA" ; OB(O)c1ccc(-n2c3cc4ccccc4cc3c3cc4ccccc4cc32)cc1 ; c1ccc2cc3c(cc2c1)c1cc2ccccc2cc1n3-c1ccc(-c2nc3c(ccc4oc5ccccc5c43)o2)cc1]

Compound AA (15 g, 61.6 mmol) and sub9 (25 g, 64.6 mmol) were added to THF (300 ml), and the mixture was stirred and refluxed. Then, potassium carbonate (25.5 g, 184.7 mmol) was dissolved in water (77 ml) and added thereto, and the mixture was sufficiently stirred, and then bis(tri-tert-butylphosphine)palladium(0) (0.3 g, 0.6 mmol) was added. After reacting for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 9 (21.7 g). (Yield: 64%, MS: [M+H]+=551)

### Example 10: Preparation of Compound 10

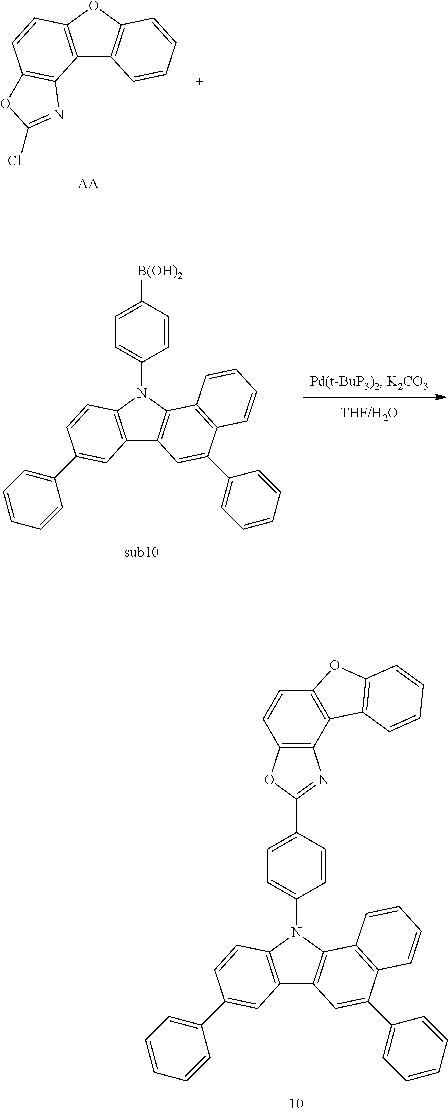 [SMILES: Clc1nc2c(ccc3oc4ccccc4c32)o1 ; "AA" ; OB(O)c1ccc(-n2c3ccc(-c4ccccc4)cc3c3cc(-c4ccccc4)c4ccccc4c32)cc1 ; c1ccc(-c2ccc3c(c2)c2cc(-c4ccccc4)c4ccccc4c2n3-c2ccc(-c3nc4c(ccc5oc6ccccc6c54)o3)cc2)cc1]

Compound AA (15 g, 61.6 mmol) and sub10 (31.6 g, 64.6 mmol) were added to THF (300 ml), and the mixture was stirred and refluxed. Then, potassium carbonate (25.5 g, 184.7 mmol) was dissolved in water (77 ml) and added thereto, and the mixture was sufficiently stirred, and then bis(tri-tert-butylphosphine)palladium(0) (0.3 g, 0.6 mmol) was added. After reacting for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 10 (24.9 g). (Yield: 62%, MS: [M+H]+=653)

### Example 11: Preparation of Compound 11

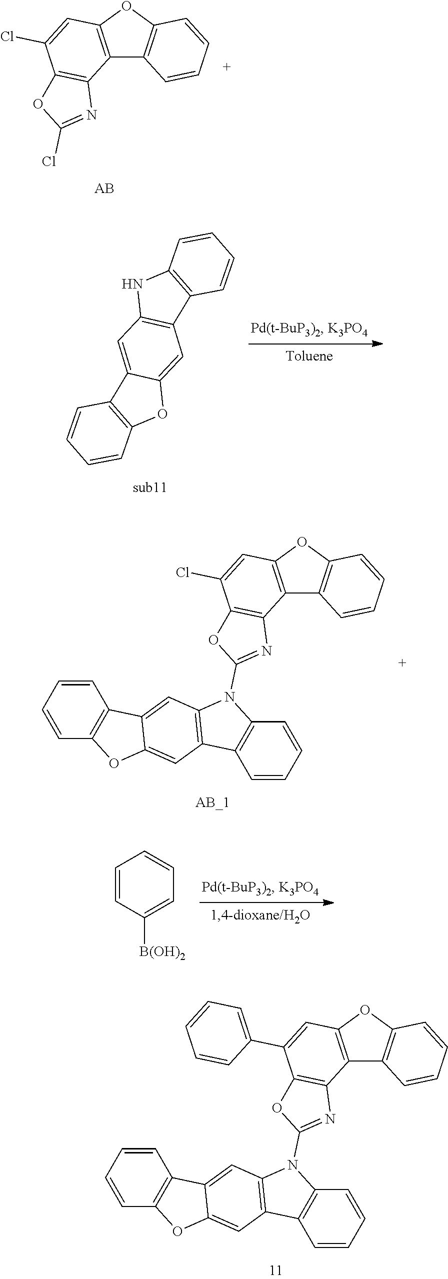 [SMILES: Clc1nc2c(o1)c(Cl)cc1oc3ccccc3c12 ; c1ccc2c(c1)[nH]c1cc3c(cc12)oc1ccccc13 ; Clc1cc2oc3ccccc3c2c2nc(-n3c4ccccc4c4cc5oc6ccccc6c5cc43)oc12 ; OB(O)c1ccccc1 ; c1ccc(-c2cc3oc4ccccc4c3c3nc(-n4c5ccccc5c5cc6oc7ccccc7c6cc54)oc23)cc1]

Compound AB (10 g, 36 mmol), sub11 (9.7 g, 37.8 mmol) and potassium phosphate (22.9 g, 107.9 mmol) were added to toluene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound AB_1 (10.9 g). (Yield: 61%, MS: [M+H]+=500)

Compound AB_1 (15 g, 30.1 mmol) and phenylboronic acid (3.8 g, 31.6 mmol) were added to 1,4-dioxane (300 ml), and the mixture was stirred and refluxed. Then, potassium phosphate (19.1 g, 90.2 mmol) was dissolved in water (57 ml) and added thereto, and the mixture was sufficiently stirred, and then bis(tri-tert-butylphosphine)palladium(0) (0.2 g, 0.3 mmol) was added. After reacting for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 11 (11 g). (Yield: 68%, MS: [M+H]+=541)

### Example 12: Preparation of Compound 12

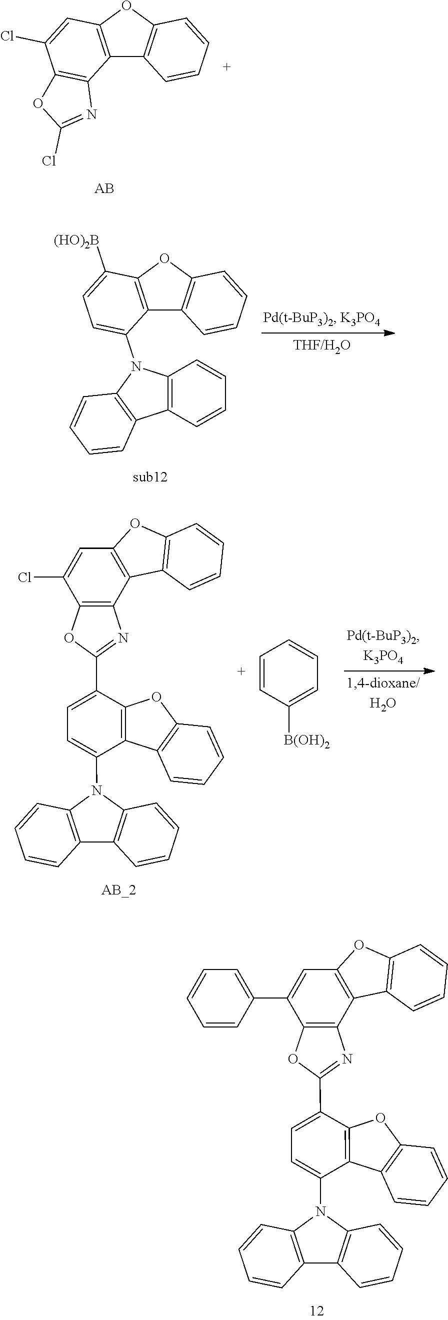 [SMILES: Clc1nc2c(o1)c(Cl)cc1oc3ccccc3c12 ; OB(O)c1ccc(-n2c3ccccc3c3ccccc32)c2c1oc1ccccc12 ; Clc1cc2oc3ccccc3c2c2nc(-c3ccc(-n4c5ccccc5c5ccccc54)c4c3oc3ccccc34)oc12 ; OB(O)c1ccccc1 ; c1ccc(-c2cc3oc4ccccc4c3c3nc(-c4ccc(-n5c6ccccc6c6ccccc65)c5c4oc4ccccc45)oc23)cc1]

Compound AB (15 g, 53.9 mmol) and sub12 (21.4 g, 56.6 mmol) were added to THF (300 ml), and the mixture was stirred and refluxed. Then, potassium carbonate (22.4 g, 161.8 mmol) was dissolved in water (67 ml) and added thereto, and the mixture was sufficiently stirred, and then bis(tri-tert-butylphosphine)palladium(0) (0.3 g, 0.5 mmol) was added. After reacting for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound AB_2 (22.3 g). (Yield: 72%, MS: [M+H]+=575)

Compound AB_2 (15 g, 26.1 mmol) and phenylboronic acid (3.3 g, 27.4 mmol) were added to 1,4-dioxane (300 ml), and the mixture was stirred and refluxed. Then, potassium phosphate (16.6 g, 78.3 mmol) was dissolved in water (50 ml) and added thereto, and the mixture was sufficiently stirred, and then bis(tri-tert-butylphosphine)palladium(0) (0.1 g, 0.3 mmol) was added. After reacting for 2 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 12 (10.9 g). (Yield: 68%, MS: [M+H]+=617)

### Example 13: Preparation of Compound 13

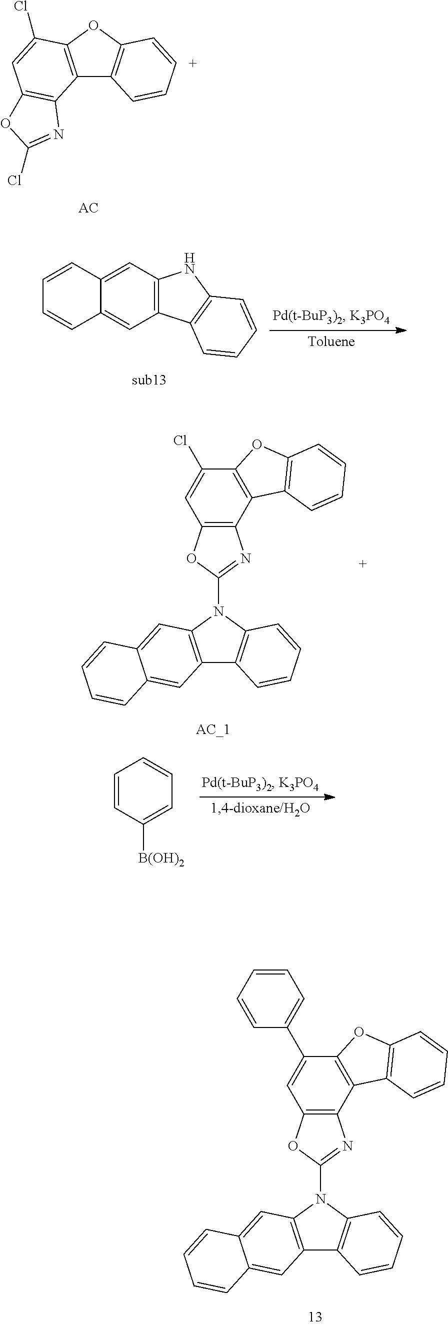 [SMILES: Clc1nc2c(cc(Cl)c3oc4ccccc4c32)o1 ; c1ccc2cc3c(cc2c1)[nH]c1ccccc13 ; Clc1cc2oc(-n3c4ccccc4c4cc5ccccc5cc43)nc2c2c1oc1ccccc12 ; OB(O)c1ccccc1 ; c1ccc(-c2cc3oc(-n4c5ccccc5c5cc6ccccc6cc54)nc3c3c2oc2ccccc23)cc1]

Compound AC (10 g, 36 mmol), sub13 (8.2 g, 37.8 mmol) and potassium phosphate (22.9 g, 107.9 mmol) were added to toluene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound AC_1 (10.7 g). (Yield: 65%, MS: [M+H]+=459)

Compound AC_1 (15 g, 32.7 mmol) and phenylboronic acid (4.2 g, 34.3 mmol) were added to 1,4-dioxane (300 ml), and the mixture was stirred and refluxed. Then, potassium phosphate (20.8 g, 98.1 mmol) was dissolved in water (62 ml) and added thereto, and the mixture was sufficiently stirred, and then bis(tri-tert-butylphosphine)palladium(0) (0.2 g, 0.3 mmol) was added. After reacting for 2 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 13 (11 g). (Yield: 67%, MS: [M+H]+=501)

### Example 14: Preparation of Compound 14

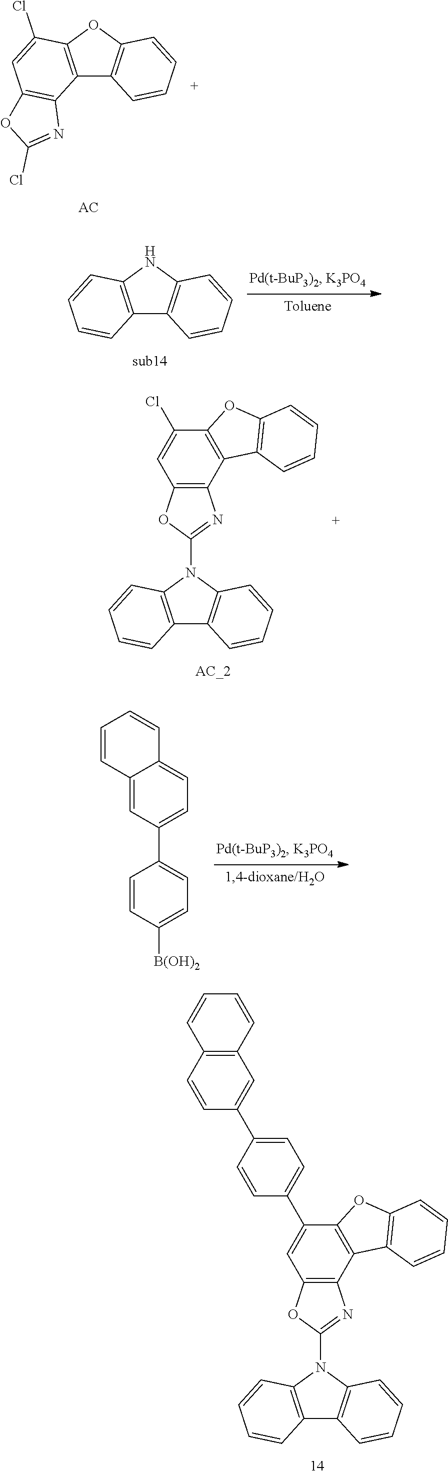 [SMILES: Clc1nc2c(cc(Cl)c3oc4ccccc4c32)o1 ; c1ccc2c(c1)[nH]c1ccccc12 ; Clc1cc2oc(-n3c4ccccc4c4ccccc43)nc2c2c1oc1ccccc12 ; OB(O)c1ccc(-c2ccc3ccccc3c2)cc1 ; c1ccc2cc(-c3ccc(-c4cc5oc(-n6c7ccccc7c7ccccc76)nc5c5c4oc4ccccc45)cc3)ccc2c1]

Compound AC (10 g, 36 mmol), sub14 (6.3 g, 37.8 mmol) and potassium phosphate (22.9 g, 107.9 mmol) were added to toluene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound AC_2 (10.6 g). (Yield: 72%, MS: [M+H]+=409)

Compound AC_2 (15 g, 36.7 mmol) and (4-(naphthalen-2-yl)phenyl)boronic acid (9.6 g, 38.5 mmol) were added to 1,4-dioxane (300 ml), and the mixture was stirred and refluxed. Then, potassium phosphate (23.4 g, 110.1 mmol) was dissolved in water (70 ml) and added thereto, and the mixture was sufficiently stirred, and then bis(tri-tert-butylphosphine)palladium(0) (0.2 g, 0.4 mmol) was added. After reacting for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 14 (13.7 g). (Yield: 65%, MS: [M+H]+=577)

### Example 15: Preparation of Compound 15

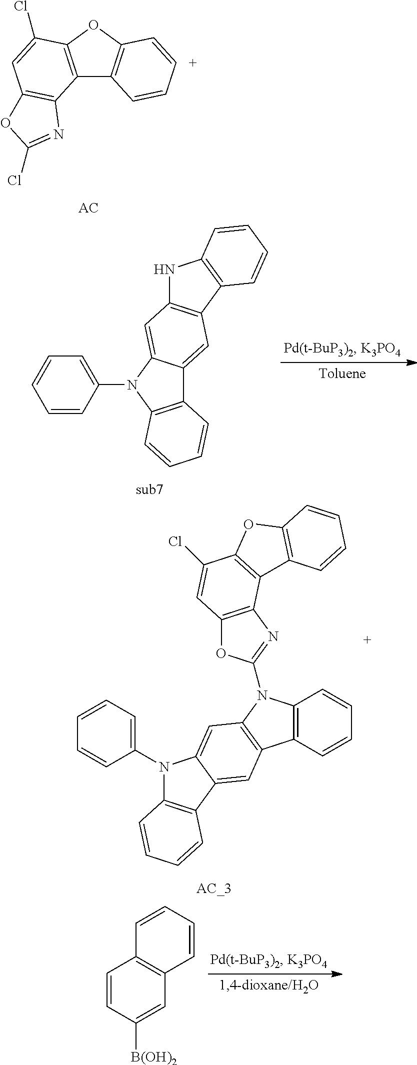

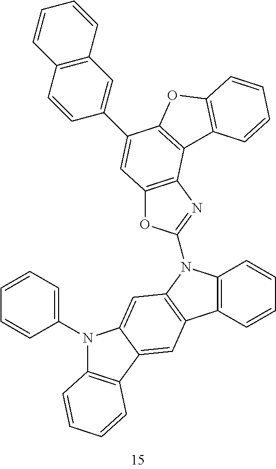 [SMILES: c1ccc(-n2c3ccccc3c3cc4c5ccccc5n(-c5nc6c(cc(-c7ccc8ccccc8c7)c7oc8ccccc8c76)o5)c4cc32)cc1]

Compound AC (10 g, 36 mmol), sub7 (6.3 g, 37.8 mmol) and potassium phosphate (22.9 g, 107.9 mmol) were added to toluene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound AC_3 (10.6 g). (Yield: 72%, MS: [M+H]+=409)

Compound AC_3 (15 g, 26.1 mmol) and naphthalen-2-ylboronic acid (3.3 g, 27.4 mmol) were added to 1,4-dioxane (300 ml), and the mixture was stirred and refluxed. Then, potassium phosphate (16.6 g, 78.4 mmol) was dissolved in water (50 ml) and added thereto, and the mixture was sufficiently stirred, and then bis(tri-tert-butylphosphine)palladium(0) (0.1 g, 0.3 mmol) was added. After reacting for 2 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 15 (11.3 g). (Yield: 65%, MS: [M+H]+=666)

### Example 16: Preparation of Compound 16

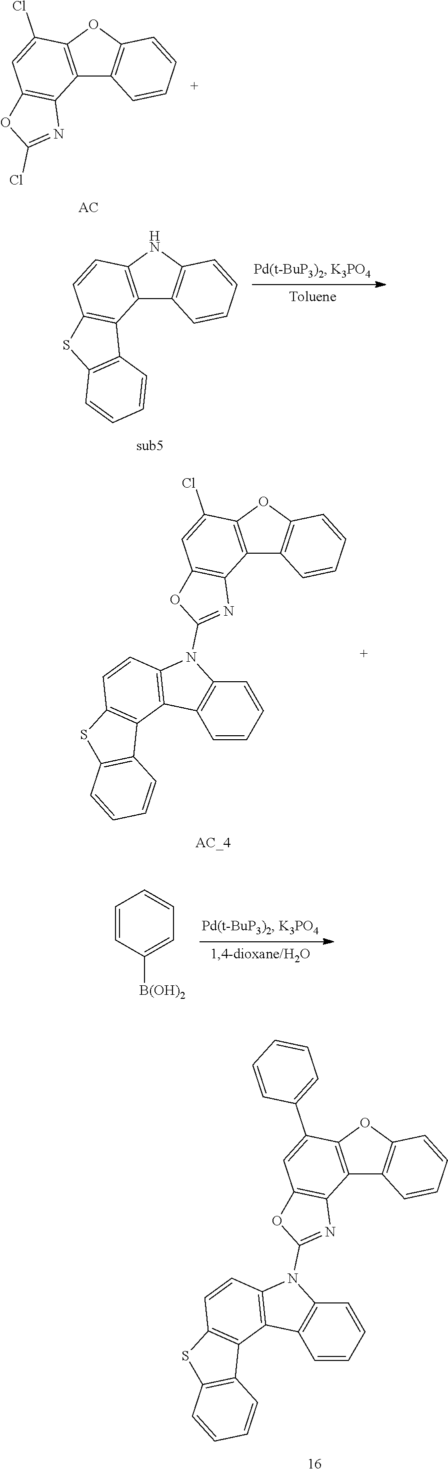 [SMILES: Clc1nc2c(cc(Cl)c3oc4ccccc4c32)o1 ; c1ccc2c(c1)[nH]c1ccc3sc4ccccc4c3c12 ; Clc1cc2oc(-n3c4ccccc4c4c5c(ccc43)sc3ccccc35)nc2c2c1oc1ccccc12 ; OB(O)c1ccccc1 ; c1ccc(-c2cc3oc(-n4c5ccccc5c5c6c(ccc54)sc4ccccc46)nc3c3c2oc2ccccc23)cc1]

Compound AC (10 g, 36 mmol), sub5 (10.3 g, 37.8 mmol) and potassium phosphate (22.9 g, 107.9 mmol) were added to toluene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound AC_4 (11.1 g). (Yield: 60%, MS: [M+H]+=515)

Compound AC_4 (15 g, 29.1 mmol) and phenylboronic acid (5.3 g, 30.6 mmol) were added to 1,4-dioxane (300 ml), and the mixture was stirred and refluxed. Then, potassium phosphate (18.5 g, 87.4 mmol) was dissolved in water (56 ml) and added thereto, and the mixture was sufficiently stirred, and then bis(tri-tert-butylphosphine)palladium(0) (0.1 g, 0.3 mmol) was added. After reacting for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 16 (10.2 g). (Yield: 63%, MS: [M+H]+=557)

### Example 17: Preparation of Compound 17

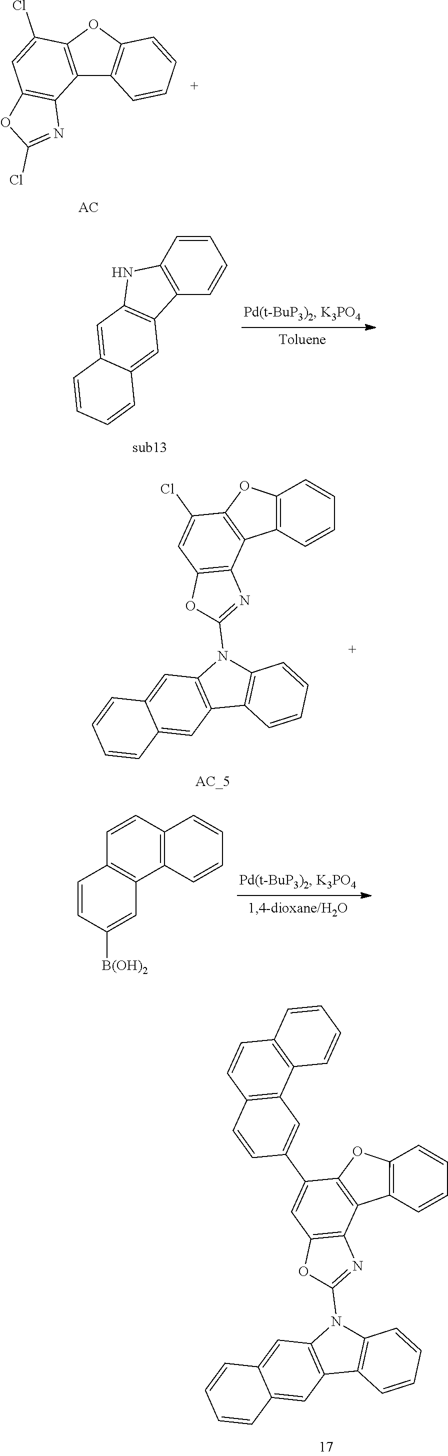 [SMILES: Clc1nc2c(cc(Cl)c3oc4ccccc4c32)o1 ; c1ccc2cc3c(cc2c1)[nH]c1ccccc13 ; Clc1cc2oc(-n3c4ccccc4c4cc5ccccc5cc43)nc2c2c1oc1ccccc12 ; OB(O)c1ccc2ccc3ccccc3c2c1 ; c1ccc2cc3c(cc2c1)c1ccccc1n3-c1nc2c(cc(-c3ccc4ccc5ccccc5c4c3)c3oc4ccccc4c32)o1]

Compound AC (10 g, 36 mmol), sub13 (8.2 g, 37.8 mmol) and potassium phosphate (22.9 g, 107.9 mmol) were added to toluene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound AC_5 (11.7 g). (Yield: 71%, MS: [M+H]+=459)

Compound AC_5 (15 g, 32.7 mmol) and phenanthren-3-ylboronic acid (8.1 g, 34.3 mmol) were added to 1,4-dioxane (300 ml), and the mixture was stirred and refluxed. Then, potassium phosphate (20.8 g, 98.1 mmol) was dissolved in water (62 ml) and added thereto, and the mixture was sufficiently stirred, and then bis(tri-tert-butylphosphine)palladium(0) (0.2 g, 0.3 mmol) was added. After reacting for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 17 (14.5 g). (Yield: 74%, MS: [M+H]+=601)

### Example 18: Preparation of Compound 18

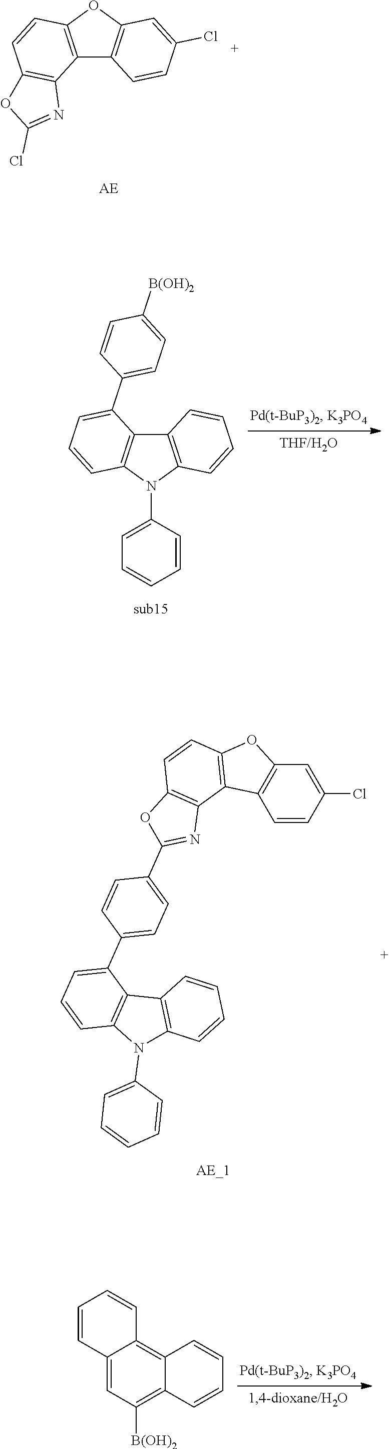 [SMILES: Clc1ccc2c(c1)oc1ccc3oc(Cl)nc3c12 ; OB(O)c1ccc(-c2cccc3c2c2ccccc2n3-c2ccccc2)cc1 ; Clc1ccc2c(c1)oc1ccc3oc(-c4ccc(-c5cccc6c5c5ccccc5n6-c5ccccc5)cc4)nc3c12 ; OB(O)c1cc2ccccc2c2ccccc12]

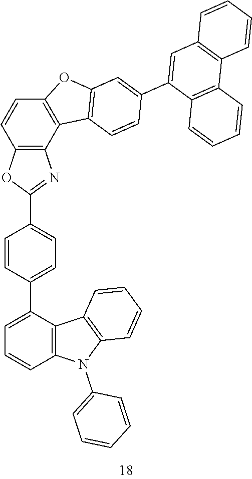 [SMILES: c1ccc(-n2c3ccccc3c3c(-c4ccc(-c5nc6c(ccc7oc8cc(-c9cc%10ccccc%10c%10ccccc9%10)ccc8c76)o5)cc4)cccc32)cc1]

Compound AE (15 g, 53.9 mmol) and sub15 (20.6 g, 56.6 mmol) were added to THF (300 ml), and the mixture was stirred and refluxed. Then, potassium carbonate (22.4 g, 161.8 mmol) was dissolved in water (67 ml) and added thereto, and the mixture was sufficiently stirred, and then bis(tri-tert-butylphosphine)palladium(0) (0.3 g, 0.5 mmol) was added. After reacting for 2 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound AE_1 (21.1 g). (Yield: 70%, MS: [M+H]+=561)

Compound AE_1 (15 g, 26.7 mmol) and phenanthren-9-ylboronic acid (6.2 g, 28.1 mmol) were added to 1,4-dioxane (300 ml), and the mixture was stirred and refluxed. Then, potassium phosphate (17 g, 80.2 mmol) was dissolved in water (51 ml) and added thereto, and the mixture was sufficiently stirred, and then bis(tri-tert-butylphosphine)palladium(0) (0.1 g, 0.3 mmol) was added. After reacting for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 18 (12 g). (Yield: 64%, MS: [M+H]+=703)

### Example 19: Preparation of Compound 19

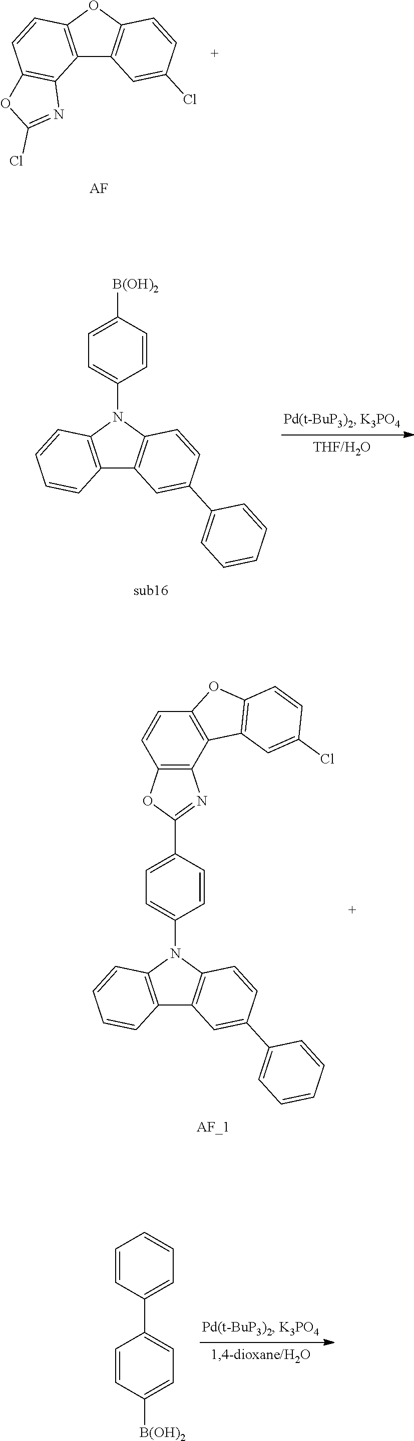 [SMILES: Clc1ccc2oc3ccc4oc(Cl)nc4c3c2c1 ; "AF" ; OB(O)c1ccc(-n2c3ccccc3c3cc(-c4ccccc4)ccc32)cc1 ; Clc1ccc2oc3ccc4oc(-c5ccc(-n6c7ccccc7c7cc(-c8ccccc8)ccc76)cc5)nc4c3c2c1 ; OB(O)c1ccc(-c2ccccc2)cc1]

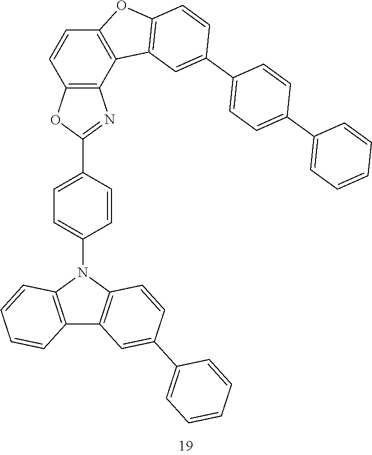 [SMILES: c1ccc(-c2ccc(-c3ccc4oc5ccc6oc(-c7ccc(-n8c9ccccc9c9cc(-c%10ccccc%10)ccc98)cc7)nc6c5c4c3)cc2)cc1]

Compound AF (15 g, 53.9 mmol) and sub16 (20.6 g, 56.6 mmol) were added to THF (300 ml), and the mixture was stirred and refluxed. Then, potassium carbonate (22.4 g, 161.8 mmol) was dissolved in water (67 ml) and added thereto, and the mixture was sufficiently stirred, and then bis(tri-tert-butylphosphine)palladium(0) (0.3 g, 0.5 mmol) was added. After reacting for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound AF_1 (21.1 g). (Yield: 70%, MS: [M+H]+=561)

Compound AF_(15 g, 26.7 mmol) and [1,1′-biphenyl]-4-ylboronic acid (5.6 g, 28.1 mmol) were added to 1,4-dioxane (300 ml), and the mixture was stirred and refluxed. Then, potassium phosphate (17 g, 80.2 mmol) was dissolved in water (51 ml) and added thereto, and the mixture was sufficiently stirred, and then bis(tri-tert-butylphosphine)palladium(0) (0.1 g, 0.3 mmol) was added. After reacting for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 19 (12.1 g). (Yield: 67%, MS: [M+H]+=679)

### Example 20: Preparation of Compound 20

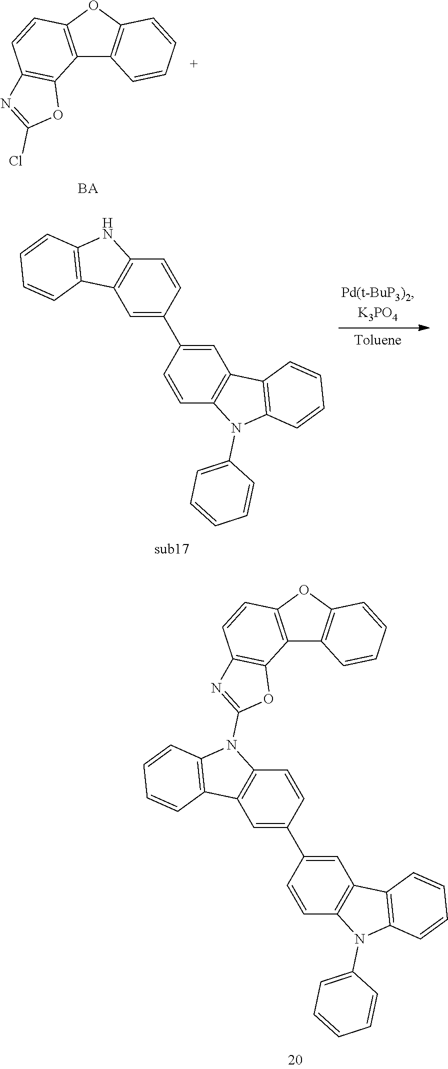 [SMILES: Clc1nc2ccc3oc4ccccc4c3c2o1 ; c1ccc(-n2c3ccccc3c3cc(-c4ccc5[nH]c6ccccc6c5c4)ccc32)cc1 ; c1ccc(-n2c3ccccc3c3cc(-c4ccc5c(c4)c4ccccc4n5-c4nc5ccc6oc7ccccc7c6c5o4)ccc32)cc1]

Compound BA (10 g, 41 mmol), sub17 (17.6 g, 43.1 mmol) and potassium phosphate (26.1 g, 123.1 mmol) were added to toluene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound 20 (17.7 g). (Yield: 70%, MS: [M+H]+=616)

### Example 21: Preparation of Compound 21

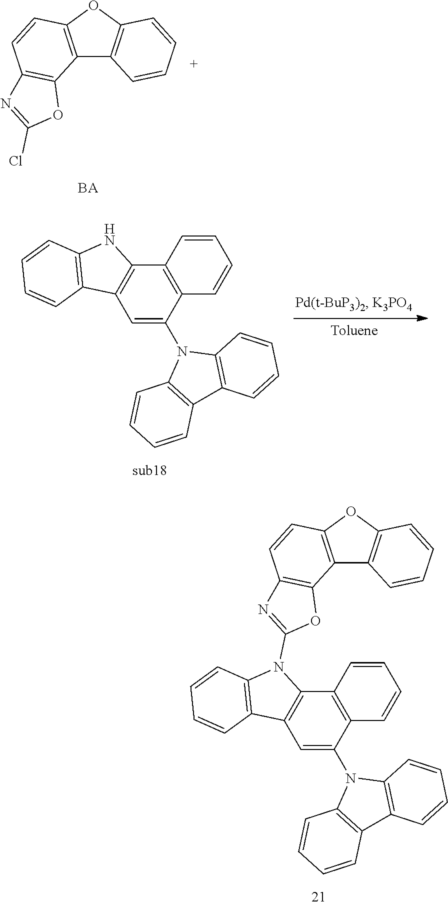 [SMILES: Clc1nc2ccc3oc4ccccc4c3c2o1 ; c1ccc2c(c1)[nH]c1c3ccccc3c(-n3c4ccccc4c4ccccc43)cc21 ; c1ccc2c(c1)oc1ccc3nc(-n4c5ccccc5c5cc(-n6c7ccccc7c7ccccc76)c6ccccc6c54)oc3c12]

Compound BA (10 g, 41 mmol), sub18 (16.5 g, 43.1 mmol) and potassium phosphate (26.1 g, 123.1 mmol) were added to toluene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound 21 (17.4 g). (Yield: 72%, MS: [M+H]+=590)

### Example 22: Preparation of Compound 22

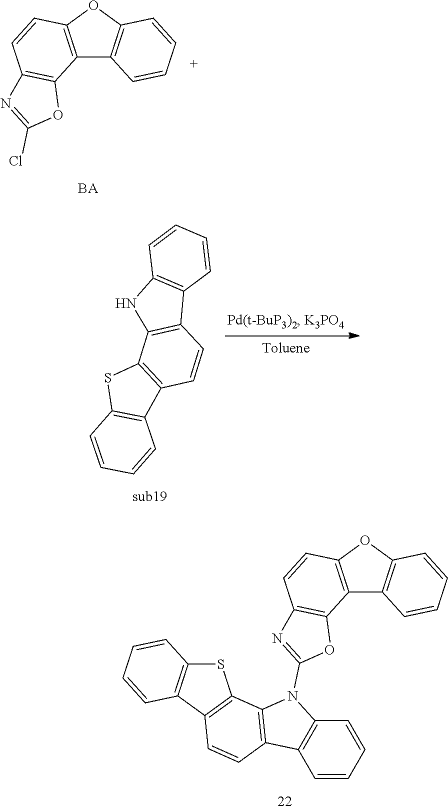 [SMILES: Clc1nc2ccc3oc4ccccc4c3c2o1 ; c1ccc2c(c1)[nH]c1c2ccc2c3ccccc3sc21 ; c1ccc2c(c1)oc1ccc3nc(-n4c5ccccc5c5ccc6c7ccccc7sc6c54)oc3c12]

Compound BA (10 g, 41 mmol), sub19 (11.8 g, 43.1 mmol) and potassium phosphate. (26.1 g, 123.1 mmol) were added to toluene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound 22 (14.6 g). (Yield: 74%, MS: [M+H]+=481)

### Example 23: Preparation of Compound 23

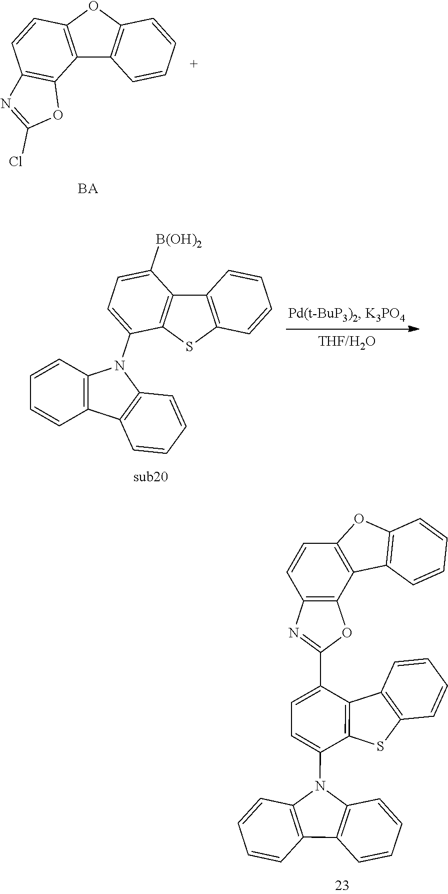 [SMILES: Clc1nc2ccc3oc4ccccc4c3c2o1 ; OB(O)c1ccc(-n2c3ccccc3c3ccccc32)c2sc3ccccc3c12 ; c1ccc2c(c1)oc1ccc3nc(-c4ccc(-n5c6ccccc6c6ccccc65)c5sc6ccccc6c45)oc3c12]

Compound BA (15 g, 61.6 mmol) and sub20 (25.4 g, 64.6 mmol) were added to THF (300 ml), and the mixture was stirred and refluxed. Then, potassium carbonate (25.5 g, 184.7 mmol) was dissolved in water (77 ml) and added thereto, and the mixture was sufficiently stirred, and then bis(tri-tert-butylphosphine)palladium(0) (0.3 g, 0.6 mmol) was added. After reacting for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 23 (21.2 g). (Yield: 62%, MS: [M+H]+=557)

### Example 24: Preparation of Compound 24

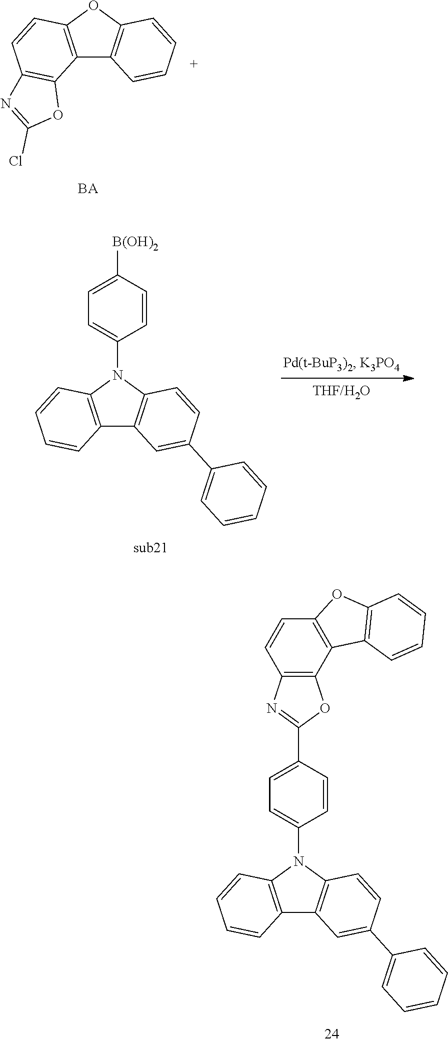 [SMILES: Clc1nc2ccc3oc4ccccc4c3c2o1 ; OB(O)c1ccc(-n2c3ccccc3c3cc(-c4ccccc4)ccc32)cc1 ; c1ccc(-c2ccc3c(c2)c2ccccc2n3-c2ccc(-c3nc4ccc5oc6ccccc6c5c4o3)cc2)cc1]

Compound BA (15 g, 61.6 mmol) and sub21 (23.5 g, 64.6 mmol) were added to THF (300 ml), and the mixture was stirred and refluxed. Then, potassium carbonate (25.5 g, 184.7 mmol) was dissolved in water (77 ml) and added thereto, and the mixture was sufficiently stirred, and then bis(tri-tert-butylphosphine)palladium(0) (0.3 g, 0.6 mmol) was added. After reacting for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 24 (24 g). (Yield: 74%, MS: [M+H]+=527)

### Example 25: Preparation of Compound 25

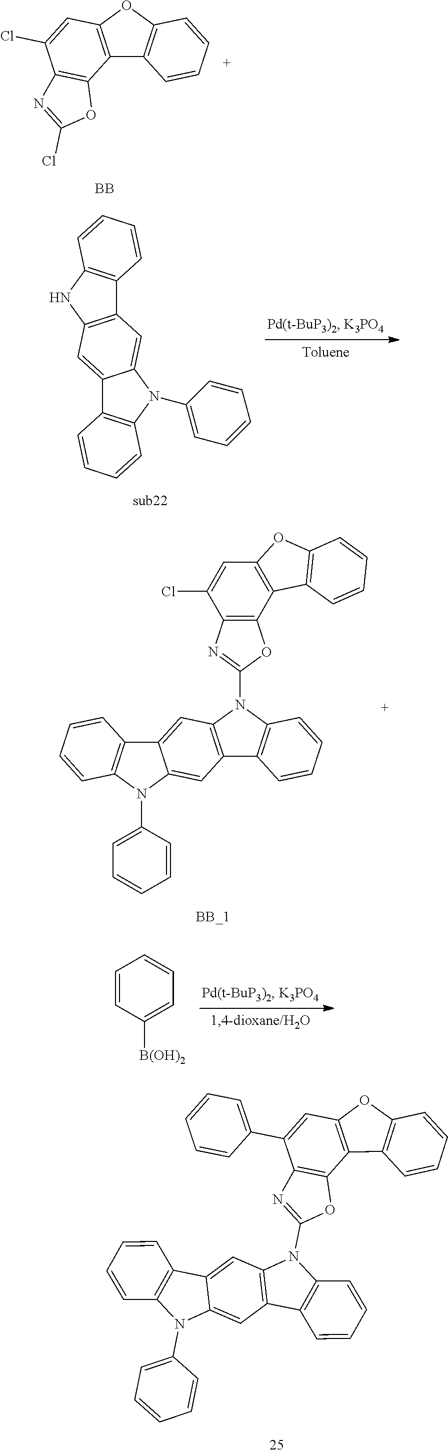

Compound BB (10 g, 36 mmol), sub22 (12.6 g, 37.8 mmol) and potassium phosphate (22.9 g, 107.9 mmol) were added to toluene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound BB_1 (13.2 g). (Yield: 64%, MS: [M+H]+=574)

Compound BB_1 (15 g, 26.1 mmol) and phenylboronic acid (3.3 g, 27.4 mmol) were added to 1,4-dioxane (300 ml), and the mixture was stirred and refluxed. Then, potassium phosphate (16.6 g, 78.4 mmol) was dissolved in water (50 ml) and added thereto, and the mixture was sufficiently stirred, and then bis(tri-tert-butylphosphine)palladium(0) (0.1 g, 0.3 mmol) was added. After reacting for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 25 (10.8 g). (Yield: 67%, MS: [M+H]+=616)

### Example 26: Preparation of Compound 26

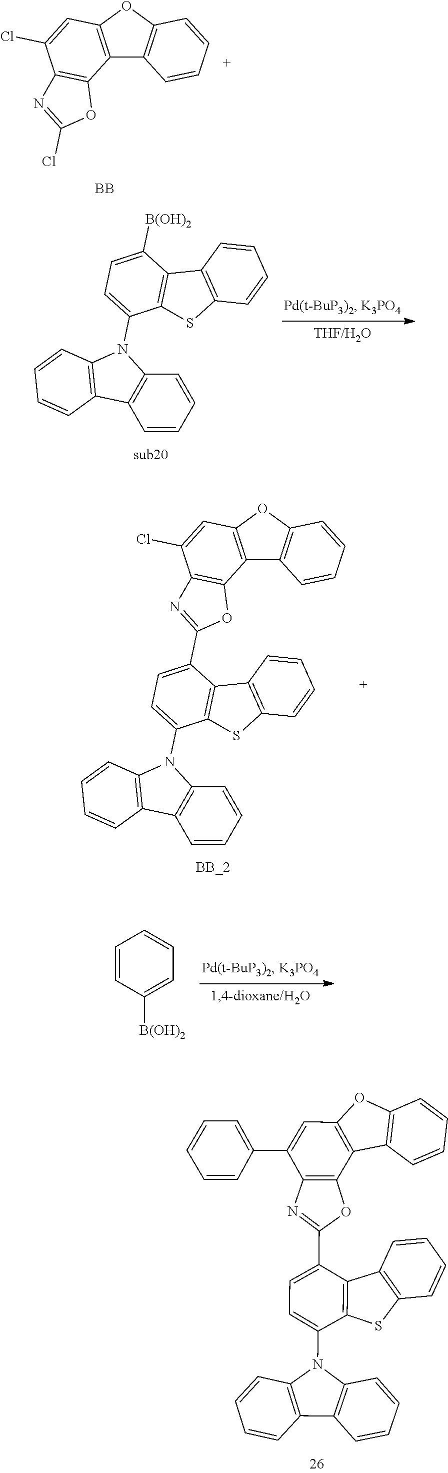

Compound BB (15 g, 53.9 mmol) and sub20 (22.3 g, 56.6 mmol) were added to THF (300 ml), and the mixture was stirred and refluxed. Then, potassium carbonate (22.4 g, 161.8 mmol) was dissolved in water (67 ml) and added thereto, and the mixture was sufficiently stirred, and then bis(tri-tert-butylphosphine)palladium(0) (0.3 g, 0.5 mmol) was added. After reacting for 2 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound BB_2 (21 g). (Yield: 66%, MS: [M+H]+=591)

Compound BB_2 (15 g, 25.4 mmol) and phenylboronic acid (3.2 g, 26.6 mmol) were added to 1,4-dioxane (300 ml), and the mixture was stirred and refluxed. Then, potassium phosphate (16.2 g, 76.1 mmol) was dissolved in water (48 ml) and added thereto, and the mixture was sufficiently stirred, and then bis(tri-tert-butylphosphine)palladium(0) (0.1 g, 0.3 mmol) was added. After reacting for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 26 (10.6 g). (Yield: 66%, MS: [M+H]+=633)

### Example 27: Preparation of Compound 27

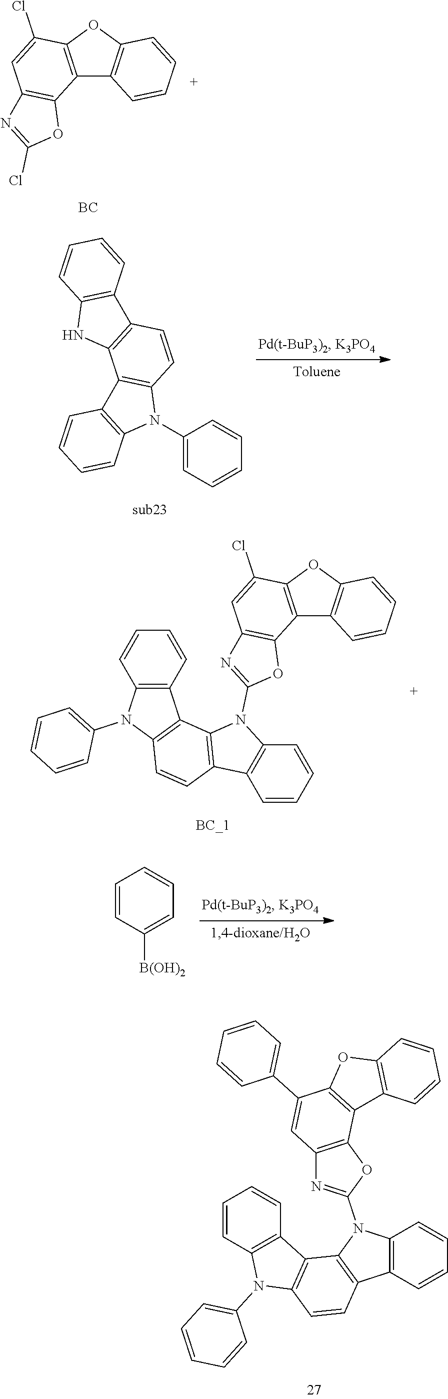

Compound BC (10 g, 36 mmol), sub23 (12.6 g, 37.8 mmol) and potassium phosphate (22.9 g, 107.9 mmol) were added to toluene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound BC_1 (13.4 g). (Yield: 65%, MS: [M+H]+=574)

Compound BC_1 (15 g, 26.1 mmol) and phenylboronic acid (3.3 g, 27.4 mmol) were added to 1,4-dioxane (300 ml), and the mixture was stirred and refluxed. Then, potassium phosphate (16.6 g, 78.4 mmol) was dissolved in water (50 ml) and added thereto, and the mixture was sufficiently stirred, and then bis(tri-tert-butylphosphine)palladium(0) (0.1 g, 0.3 mmol) was added. After reacting for 2 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 27 (10.6 g). (Yield: 66%, MS: [M+H]+=616)

### Example 28: Preparation of Compound 28

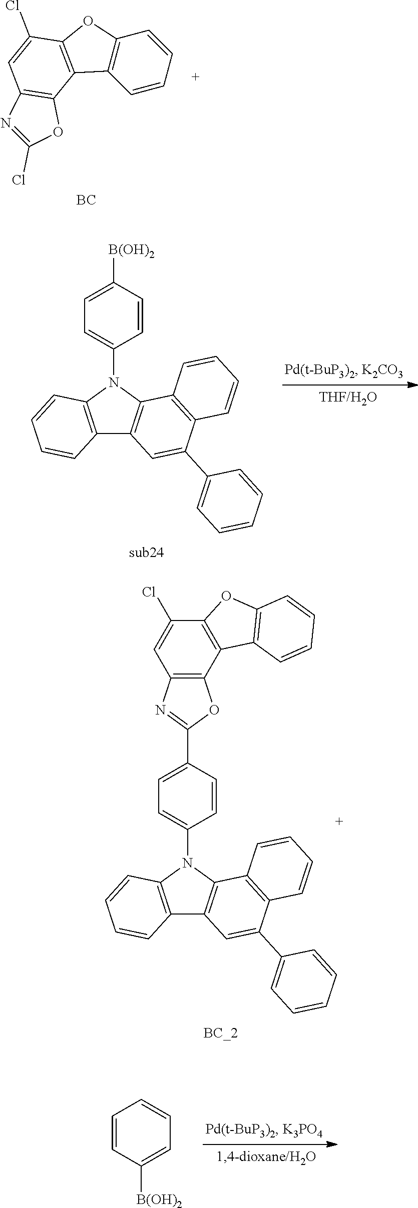

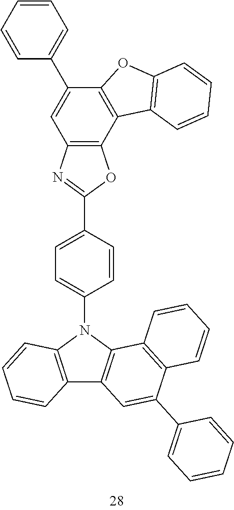 [SMILES: c1ccc(-c2cc3c4ccccc4n(-c4ccc(-c5nc6cc(-c7ccccc7)c7oc8ccccc8c7c6o5)cc4)c3c3ccccc23)cc1]

Compound BC (15 g, 53.9 mmol) and sub24 (23.4 g, 56.6 mmol) were added to THF (300 ml), and the mixture was stirred and refluxed. Then, potassium carbonate (22.4 g, 161.8 mmol) was dissolved in water (67 ml) and added thereto, and the mixture was sufficiently stirred, and then bis(tri-tert-butylphosphine)palladium(0) (0.3 g, 0.5 mmol) was added. After reacting for 2 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound BC_2 (20.4 g). (Yield: 62%, MS: [M+H]+=611)

Compound BC_2 (15 g, 24.5 mmol) and phenylboronic acid (3.1 g, 25.8 mmol) were added to 1,4-dioxane (300 ml), and the mixture was stirred and refluxed. Then, potassium phosphate (15.6 g, 73.6 mmol) was dissolved in water (47 ml) and added thereto, and the mixture was sufficiently stirred, and then bis(tri-tert-butylphosphine)palladium(0) (0.1 g, 0.2 mmol) was added. After reacting for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 28 (11.8 g). (Yield: 74%, MS: [M+H]+=653)

### Example 29: Preparation of Compound 29

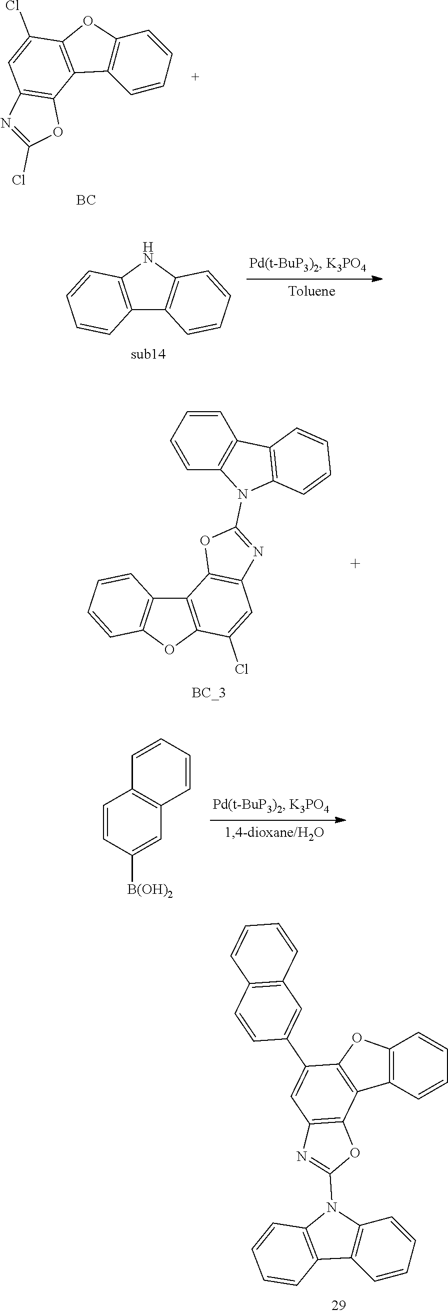

Compound BC (10 g, 36 mmol), sub14 (6.3 g, 37.8 mmol) and potassium phosphate (22.9 g, 107.9 mmol) were added to toluene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound BC_3 (11 g). (Yield: 75%, MS: [M+H]+=409)

Compound BC_3 (15 g, 36.7 mmol) and naphthalen-2-ylboronic acid (6.6 g, 38.5 mmol) were added to 1,4-dioxane (300 ml), and the mixture was stirred and refluxed. Then, potassium phosphate (23.4 g, 110.1 mmol) was dissolved in water (70 ml) and added thereto, and the mixture was sufficiently stirred, and then bis(tri-tert-butylphosphine)palladium(0) (0.2 g, 0.4 mmol) was added. After reacting for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 29 (13.4 g). (Yield: 73%, MS: [M+H]+=501)

### Example 30: Preparation of Compound 30

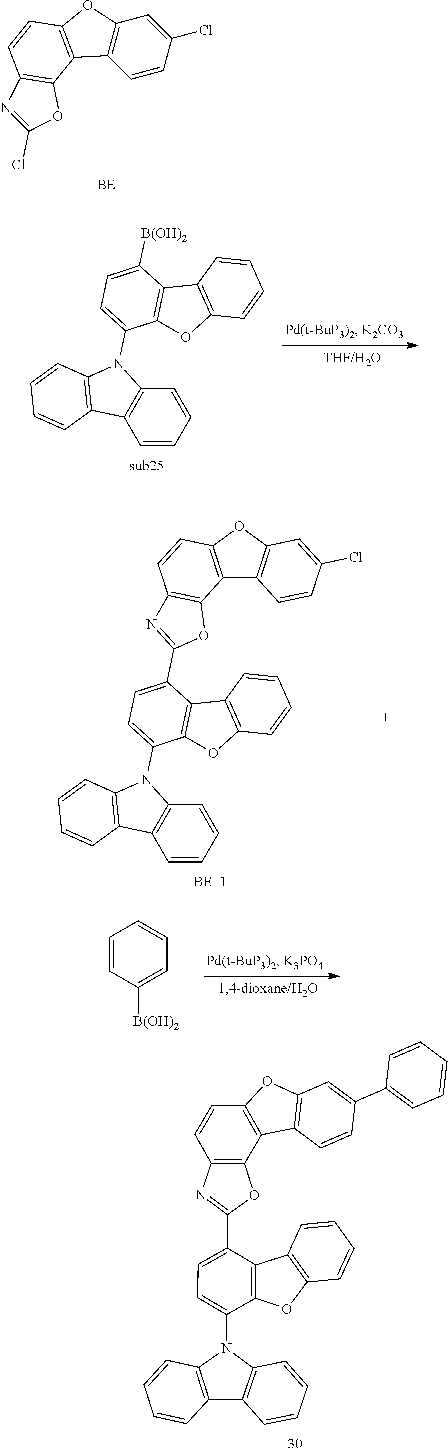 [SMILES: Clc1ccc2c(c1)oc1ccc3nc(Cl)oc3c12 ; "BE" ; OB(O)c1ccc(-n2c3ccccc3c3ccccc32)c2oc3ccccc3c12 ; Clc1ccc2c(c1)oc1ccc3nc(-c4ccc(-n5c6ccccc6c6ccccc65)c5oc6ccccc6c45)oc3c12 ; OB(O)c1ccccc1 ; c1ccc(-c2ccc3c(c2)oc2ccc4nc(-c5ccc(-n6c7ccccc7c7ccccc76)c6oc7ccccc7c56)oc4c23)cc1]

Compound BE (15 g, 53.9 mmol) and sub25 (21.4 g, 56.6 mmol) were added to THF (300 ml), and the mixture was stirred and refluxed. Then, potassium carbonate (22.4 g, 161.8 mmol) was dissolved in water (67 ml) and added thereto, and the mixture was sufficiently stirred, and then bis(tri-tert-butylphosphine)palladium(0) (0.3 g, 0.5 mmol) was added. After reacting for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound BE_1 (20.1 g). (Yield: 65%, MS: [M+H]+=575)

Compound BE_1 (15 g, 26.1 mmol) and phenylboronic acid (3.3 g, 27.4 mmol) were added to 1,4-dioxane (300 ml), and the mixture was stirred and refluxed. Then, potassium phosphate (16.6 g, 78.3 mmol) was dissolved in water (50 ml) and added thereto, and the mixture was sufficiently stirred, and then bis(tri-tert-butylphosphine)palladium(0) (0.1 g, 0.3 mmol) was added. After reacting for 2 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 30 (10 g). (Yield: 62%, MS: [M+H]+=617)

### Example 31: Preparation of Compound 31

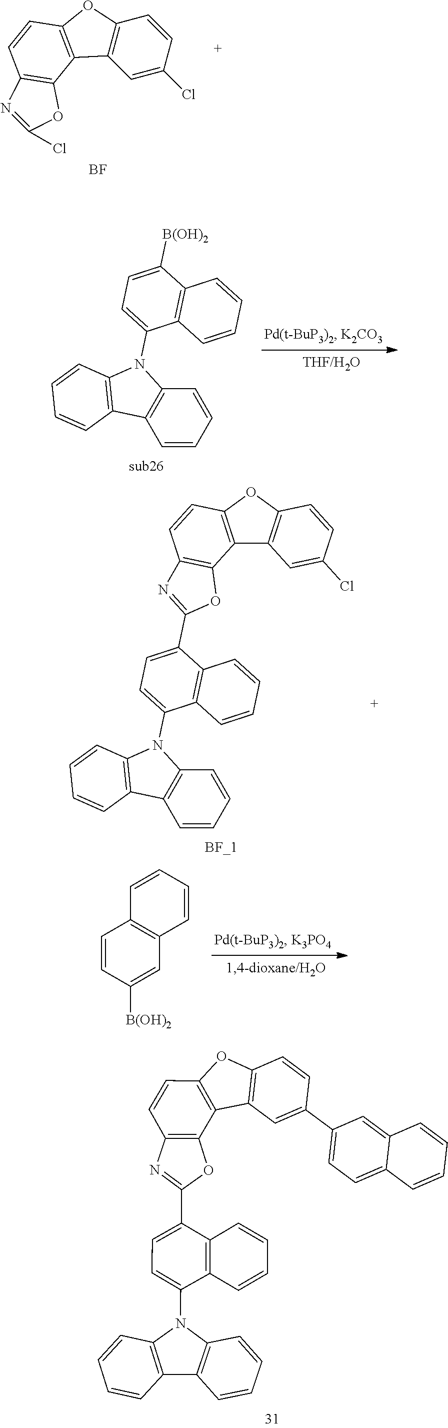

Compound BF (15 g, 53.9 mmol) and sub26 (19.1 g, 56.6 mmol) were added to THF (300 ml), and the mixture was stirred and refluxed. Then, potassium carbonate (22.4 g, 161.8 mmol) was dissolved in water (67 ml) and added thereto, and the mixture was sufficiently stirred, and then bis(tri-tert-butylphosphine)palladium(0) (0.3 g, 0.5 mmol) was added. After reacting for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound BF_1 (20.2 g). (Yield: 70%, MS: [M+H]+=535)

Compound BF_1 (15 g, 28 mmol) and naphthalen-2-ylboronic acid (5.1 g, 29.4 mmol) were added to 1,4-dioxane (300 ml), and the mixture was stirred and refluxed. Then, potassium phosphate (17.9 g, 84.1 mmol) was dissolved in water (54 ml) and added thereto, and the mixture was sufficiently stirred, and then bis(tri-tert-butylphosphine)palladium(0) (0.1 g, 0.3 mmol) was added. After reacting for 2 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 31 (11.9 g). (Yield: 68%, MS: [M+H]+=627)

### Example 32: Preparation of Compound 32

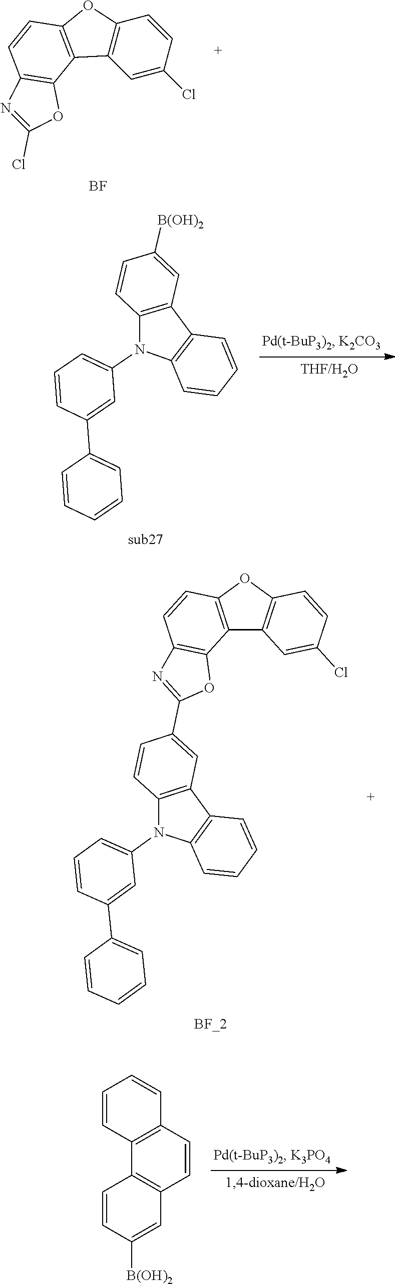 [SMILES: Clc1ccc2oc3ccc4nc(Cl)oc4c3c2c1 ; "BF" ; OB(O)c1ccc2c(c1)c1ccccc1n2-c1cccc(-c2ccccc2)c1 ; Clc1ccc2oc3ccc4nc(-c5ccc6c(c5)c5ccccc5n6-c5cccc(-c6ccccc6)c5)oc4c3c2c1 ; OB(O)c1ccc2c(ccc3ccccc32)c1]

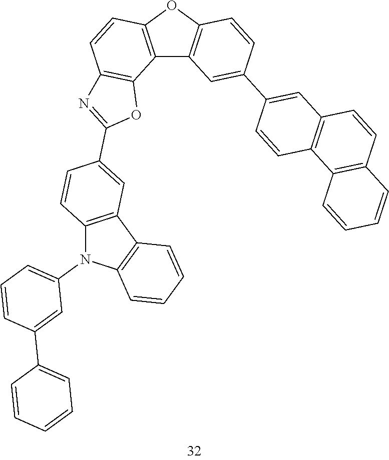 [SMILES: c1ccc(-c2cccc(-n3c4ccccc4c4cc(-c5nc6ccc7oc8ccc(-c9ccc%10c(ccc%11ccccc%11%10)c9)cc8c7c6o5)ccc43)c2)cc1]

Compound BF (15 g, 53.9 mmol) and sub27 (20.6 g, 56.6 mmol) were added to THF (300 ml), and the mixture was stirred and refluxed. Then, potassium carbonate (22.4 g, 161.8 mmol) was dissolved in water (67 ml) and added thereto, and the mixture was sufficiently stirred, and then bis(tri-tert-butylphosphine)palladium(0) (0.3 g, 0.5 mmol) was added. After reacting for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound BF_2 (20.2 g). (Yield: 67%, MS: [M+H]+=561)

Compound BF_2 (15 g, 26.7 mmol) and phenanthren-2-ylboronic acid (6.2 g, 28.1 mmol) were added to 1,4-dioxane (300 ml), and the mixture was stirred and refluxed. Then, potassium phosphate (17 g, 80.2 mmol) was dissolved in water (51 ml) and added thereto, and the mixture was sufficiently stirred, and then bis(tri-tert-butylphosphine)palladium(0) (0.1 g, 0.3 mmol) was added. After reacting for 2 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 32 (13.5 g). (Yield: 72%, MS: [M+H]+=703)

### Example 33: Preparation of Compound 33

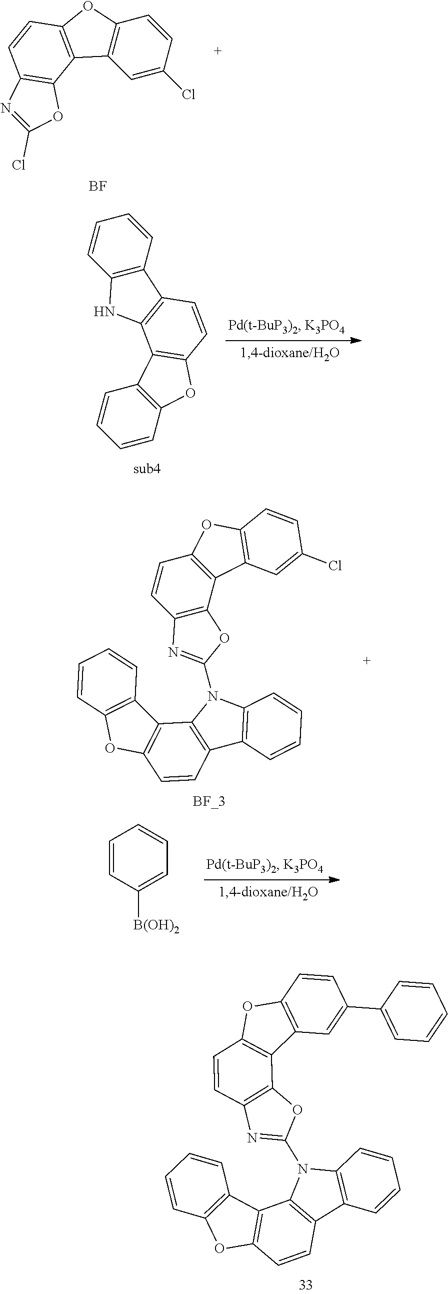

Compound BF (10 g, 36 mmol), sub4 (9.7 g, 37.8 mmol) and potassium phosphate (22.9 g, 107.9 mmol) were added to toluene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound BF_3 (11.8 g). (Yield: 66%, MS: [M+H]+=499)

Compound BF_3 (15 g, 30.1 mmol) and phenylboronic acid (3.8 g, 31.6 mmol) were added to 1,4-dioxane (300 ml), and the mixture was stirred and refluxed. Then, potassium phosphate (19.1 g, 90.2 mmol) was dissolved in water (57 ml) and added thereto, and the mixture was sufficiently stirred, and then bis(tri-tert-butylphosphine)palladium(0) (0.2 g, 0.3 mmol) was added. After reacting for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 33 (11.2 g). (Yield: 69%, MS: [M+H]+=541)

### Example 34: Preparation of Compound 34

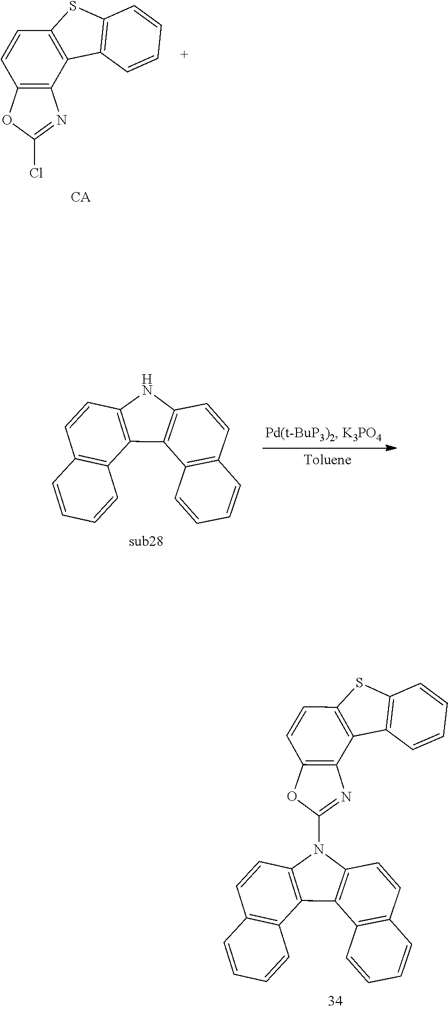 [SMILES: Clc1nc2c(ccc3sc4ccccc4c32)o1 ; c1ccc2c(c1)ccc1[nH]c3ccc4ccccc4c3c12 ; c1ccc2c(c1)ccc1c2c2c3ccccc3ccc2n1-c1nc2c(ccc3sc4ccccc4c32)o1]

Compound CA (10 g, 38.5 mmol), sub28 (10.8 g, 40.4 mmol) and potassium phosphate (24.5 g, 115.5 mmol) were added to toluene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound 34 (12.8 g). (Yield: 68%, MS: [M+H]+=491)

### Example 35: Preparation of Compound 35

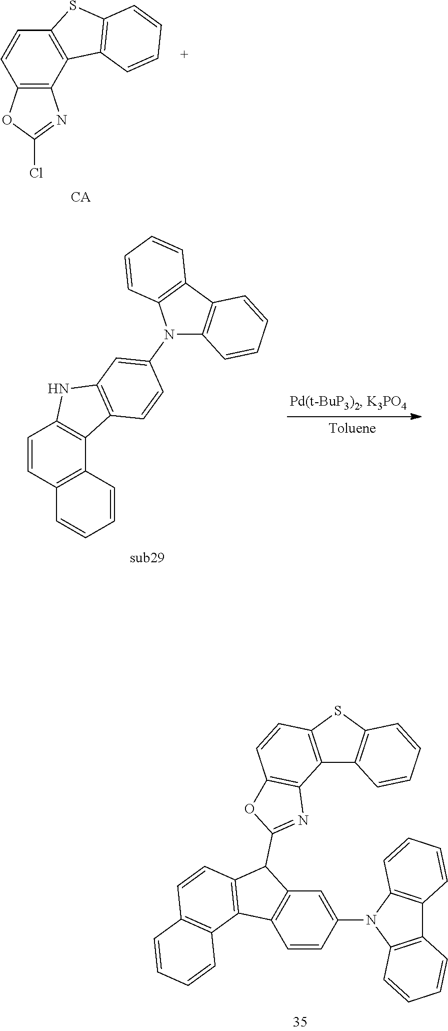 [SMILES: Clc1nc2c(ccc3sc4ccccc4c32)o1 ; c1ccc2c(c1)ccc1[nH]c3cc(-n4c5ccccc5c5ccccc54)ccc3c12 ; c1ccc2c3c(ccc2c1)C(c1nc2c(ccc4sc5ccccc5c42)o1)c1cc(-n2c4ccccc4c4ccccc42)ccc1-3]

Compound CA (10 g, 38.5 mmol), sub29 (15.5 g, 40.4 mmol) and potassium phosphate (24.5 g, 115.5 mmol) were added to toluene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound 35 (15.6 g). (Yield: 67%, MS: [M+H]+=606)

### Example 36: Preparation of Compound 36

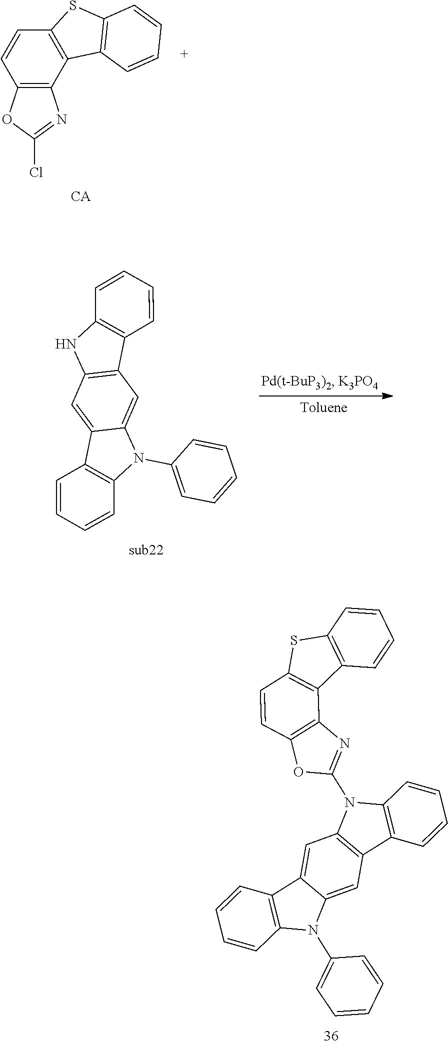 [SMILES: Clc1nc2c(ccc3sc4ccccc4c32)o1 ; c1ccc(-n2c3ccccc3c3cc4[nH]c5ccccc5c4cc32)cc1 ; c1ccc(-n2c3ccccc3c3cc4c(cc32)c2ccccc2n4-c2nc3c(ccc4sc5ccccc5c43)o2)cc1]

Compound CA (10 g, 38.5 mmol), sub22 (13.4 g, 40.4 mmol) and potassium phosphate (24.5 g, 115.5 mmol) were added to toluene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound 36 (13.3 g). (Yield: 62%, MS: [M+H]+=556)

### Example 37: Preparation of Compound 37

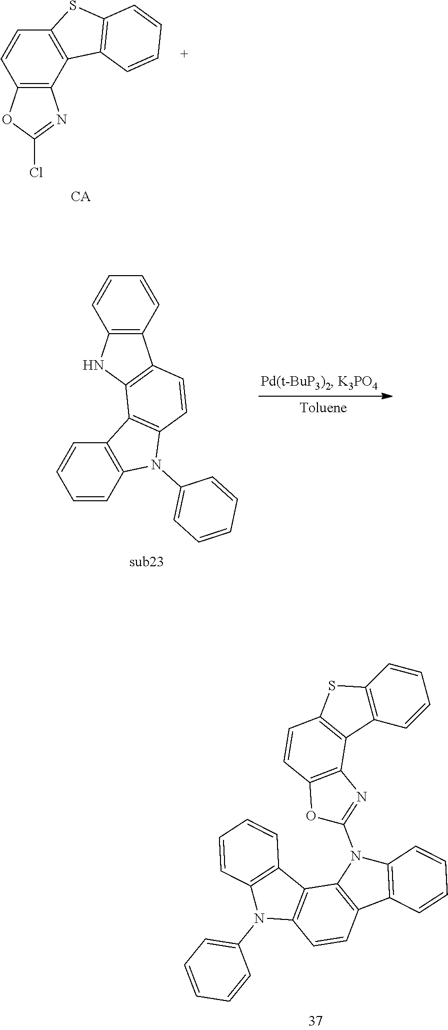 [SMILES: Clc1nc2c(ccc3sc4ccccc4c32)o1 ; c1ccc(-n2c3ccccc3c3c4[nH]c5ccccc5c4ccc32)cc1 ; c1ccc(-n2c3ccccc3c3c2ccc2c4ccccc4n(-c4nc5c(ccc6sc7ccccc7c65)o4)c23)cc1]

Compound CA (10 g, 38.5 mmol), sub23 (13.4 g, 40.4 mmol) and potassium phosphate (24.5 g, 115.5 mmol) were added to toluene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound 37 (14.1 g). (Yield: 66%, MS: [M+H]+=556)

### Example 38: Preparation of Compound 38

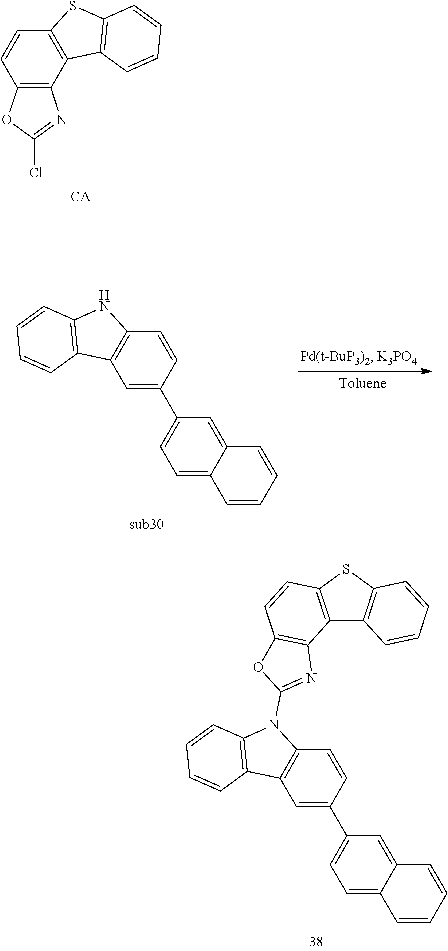 [SMILES: Clc1nc2c(ccc3sc4ccccc4c32)o1 ; c1ccc2cc(-c3ccc4[nH]c5ccccc5c4c3)ccc2c1 ; c1ccc2cc(-c3ccc4c(c3)c3ccccc3n4-c3nc4c(ccc5sc6ccccc6c54)o3)ccc2c1]

Compound CA (10 g, 38.5 mmol), sub30 (11.9 g, 40.4 mmol) and potassium phosphate (24.5 g, 115.5 mmol) were added to toluene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound 38 (11.9 g). (Yield: 60%, MS: [M+H]+=517)

### Example 39: Preparation of Compound 39

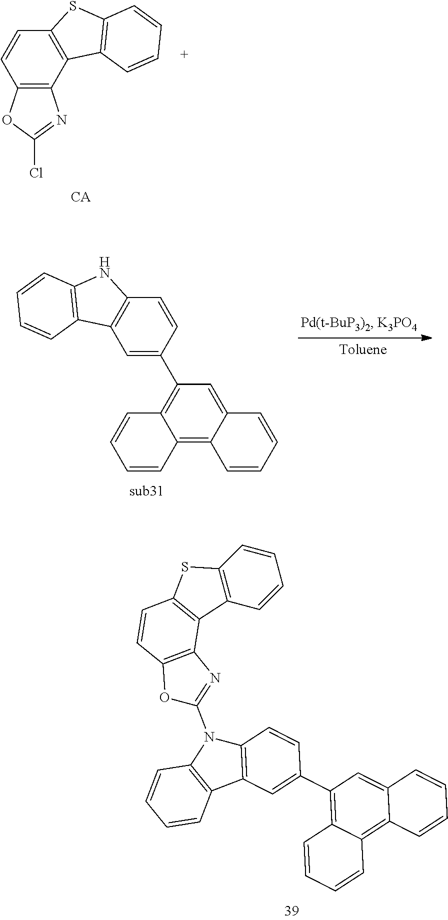 [SMILES: Clc1nc2c(ccc3sc4ccccc4c32)o1 ; c1ccc2c(c1)cc(-c1ccc3[nH]c4ccccc4c3c1)c1ccccc12 ; c1ccc2c(c1)cc(-c1ccc3c(c1)c1ccccc1n3-c1nc3c(ccc4sc5ccccc5c43)o1)c1ccccc12]

Compound CA (10 g, 38.5 mmol), sub31 (13.9 g, 40.4 mmol) and potassium phosphate (24.5 g, 115.5 mmol) were added to toluene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound 39 (15.7 g). (Yield: 72%, MS: [M+H]+=567)

### Example 40: Preparation of Compound 40

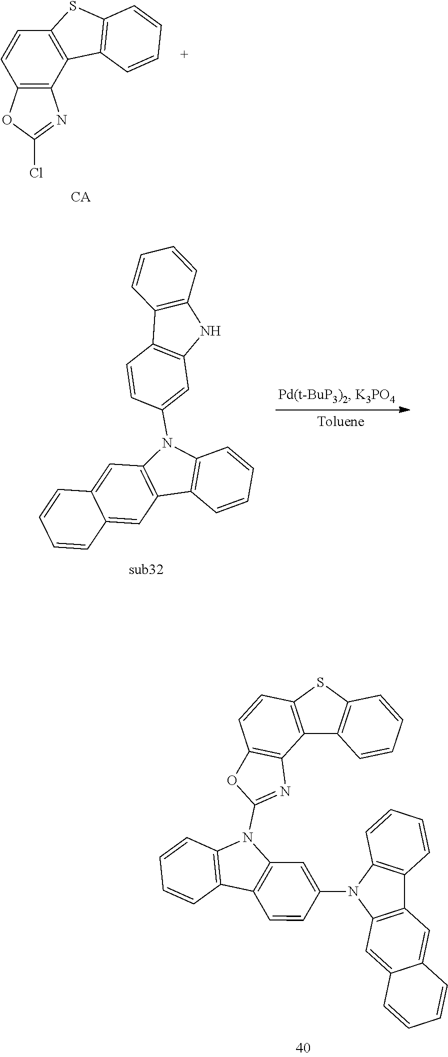 [SMILES: Clc1nc2c(ccc3sc4ccccc4c32)o1 ; c1ccc2cc3c(cc2c1)c1ccccc1n3-c1ccc2c(c1)[nH]c1ccccc12 ; c1ccc2cc3c(cc2c1)c1ccccc1n3-c1ccc2c3ccccc3n(-c3nc4c(ccc5sc6ccccc6c54)o3)c2c1]

Compound CA (10 g, 38.5 mmol), sub32 (15.5 g, 40.4 mmol) and potassium phosphate (24.5 g, 115.5 mmol) were added to toluene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound 40 (15.4 g). (Yield: 66%, MS: [M+H]+=606)

### Example 41: Preparation of Compound 41

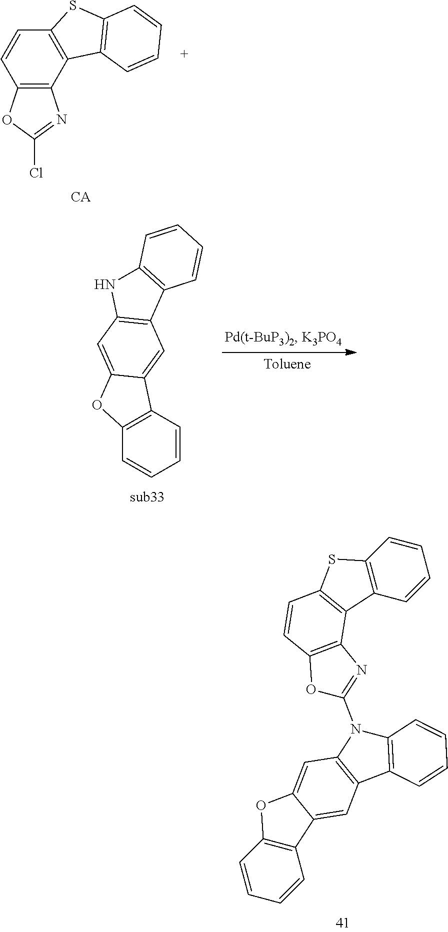 [SMILES: Clc1nc2c(ccc3sc4ccccc4c32)o1 ; c1ccc2c(c1)[nH]c1cc3oc4ccccc4c3cc12 ; c1ccc2c(c1)oc1cc3c(cc12)c1ccccc1n3-c1nc2c(ccc3sc4ccccc4c32)o1]

Compound CA (10 g, 38.5 mmol), sub33 (10.4 g, 40.4 mmol) and potassium phosphate (24.5 g, 115.5 mmol) were added to toluene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound 41 (13.7 g). (Yield: 74%, MS: [M+H]+=481)

### Example 42: Preparation of Compound 42

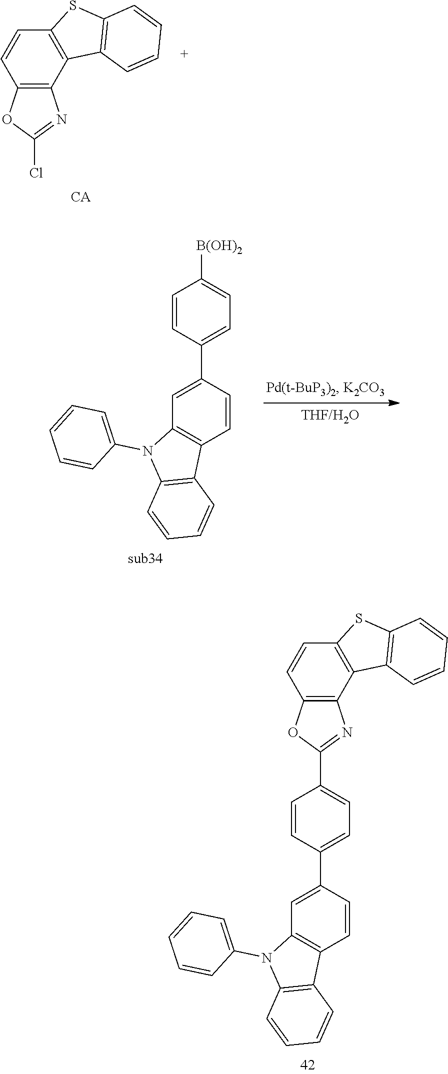 [SMILES: Clc1nc2c(ccc3sc4ccccc4c32)o1 ; OB(O)c1ccc(-c2ccc3c4ccccc4n(-c4ccccc4)c3c2)cc1 ; c1ccc(-n2c3ccccc3c3ccc(-c4ccc(-c5nc6c(ccc7sc8ccccc8c76)o5)cc4)cc32)cc1]

Compound CA (15 g, 57.8 mmol) and sub34 (22 g, 60.6 mmol) were added to THF (300 ml), and the mixture was stirred and refluxed. Then, potassium carbonate (23.9 g, 173.3 mmol) was dissolved in water (72 ml) and added thereto, and the mixture was sufficiently stirred, and then bis(tri-tert-butylphosphine)palladium(0) (0.3 g, 0.6 mmol) was added. After reacting for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 42 (21 g). (Yield: 67%, MS: [M+H]+=543)

### Example 43: Preparation of Compound 43

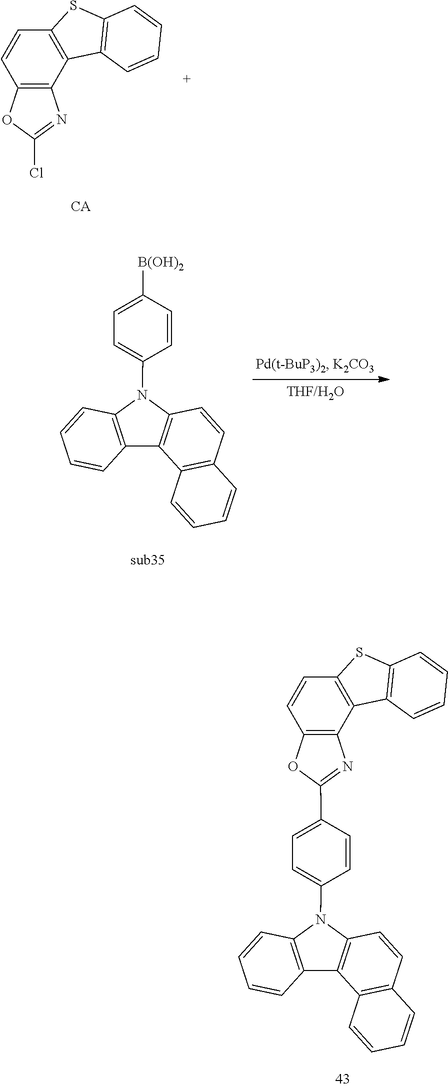 [SMILES: Clc1nc2c(ccc3sc4ccccc4c32)o1 ; OB(O)c1ccc(-n2c3ccccc3c3c4ccccc4ccc32)cc1 ; c1ccc2c(c1)ccc1c2c2ccccc2n1-c1ccc(-c2nc3c(ccc4sc5ccccc5c43)o2)cc1]

Compound CA (15 g, 57.8 mmol) and sub35 (20.4 g, 60.6 mmol) were added to THF (300 ml), and the mixture was stirred and refluxed. Then, potassium carbonate (23.9 g, 173.3 mmol) was dissolved in water (72 ml) and added thereto, and the mixture was sufficiently stirred, and then bis(tri-tert-butylphosphine)palladium(0) (0.3 g, 0.6 mmol) was added. After reacting for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 43 (18.5 g). (Yield: 62%, MS: [M+H]+=517)

### Example 44: Preparation of Compound 44

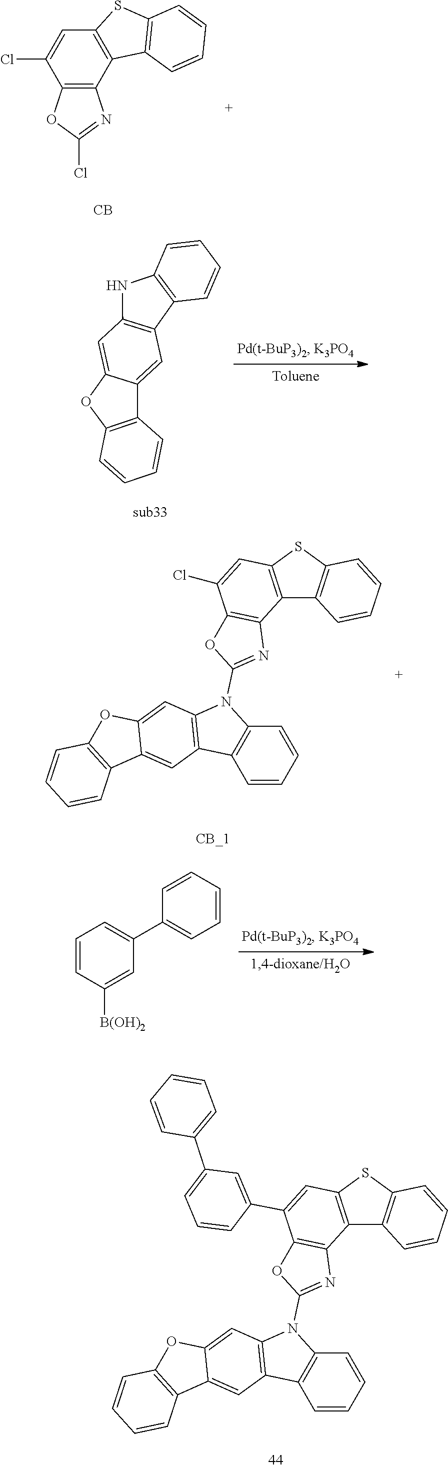

Compound CB (10 g, 34 mmol), sub33 (9.2 g, 35.7 mmol) and potassium phosphate (21.6 g, 102 mmol) were added to toluene (200 ml) under a nitrogen atmosphere, and the mixture was stirred and refluxed. Then, bis(tri-tert-butylphosphine)palladium(0) (0.2 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound CB_1 (12.9 g). (Yield: 74%, MS: [M+H]+=515)

Compound CB_1 (15 g, 29.1 mmol) and [1,1′-biphenyl]-3-ylboronic acid (6.1 g, 30.6 mmol) were added to 1,4-dioxane (300 ml), and the mixture was stirred and refluxed. Then, potassium phosphate (18.5 g, 87.4 mmol) was dissolved in water (56 ml) and added thereto, and the mixture was sufficiently stirred, and then bis(tri-tert-butylphosphine)palladium(0) (0.1 g, 0.3 mmol) was added. After reacting for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 44 (13.1 g). (Yield: 71%, MS: [M+H]+=633)

### Example 45: Preparation of Compound 45

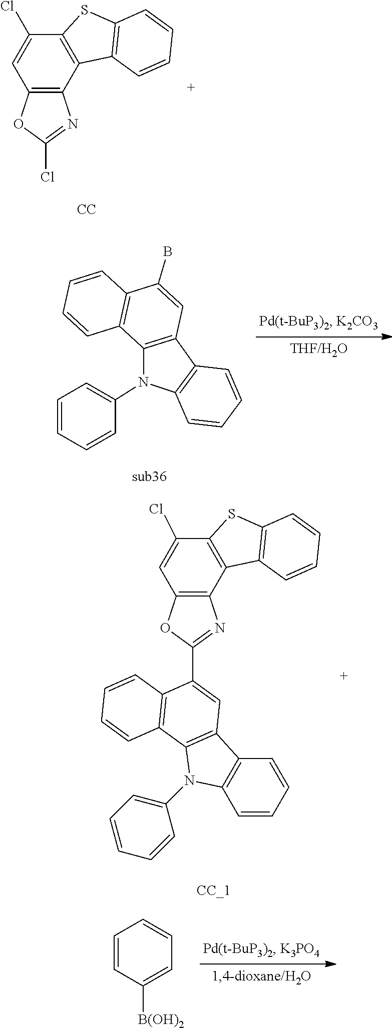

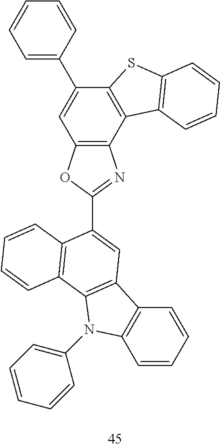 [SMILES: c1ccc(-c2cc3oc(-c4cc5c6ccccc6n(-c6ccccc6)c5c5ccccc45)nc3c3c2sc2ccccc23)cc1]

Compound CC (15 g, 51 mmol) and sub36 (18.1 g, 53.5 mmol) were added to THF (300 ml), and the mixture was stirred and refluxed. Then, potassium carbonate (21.1 g, 153 mmol) was dissolved in water (63 ml) and added thereto, and the mixture was sufficiently stirred, and then bis(tri-tert-butylphosphine)palladium(0) (0.3 g, 0.5 mmol) was added. After reacting for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound CC_1 (18.8 g). (Yield: 67%, MS: [M+H]+=551)

Compound CC_1 (15 g, 27.2 mmol) and phenylboronic acid (3.5 g, 28.6 mmol) were added to 1,4-dioxane (300 ml), and the mixture was stirred and refluxed. Then, potassium phosphate (17.3 g, 81.7 mmol) was dissolved in water (52 ml) and added thereto, and the mixture was sufficiently stirred, and then bis(tri-tert-butylphosphine)palladium(0) (0.1 g, 0.3 mmol) was added. After reacting for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 45 (11.9 g). (Yield: 74%, MS: [M+H]+=593)

### Example 46: Preparation of Compound 46

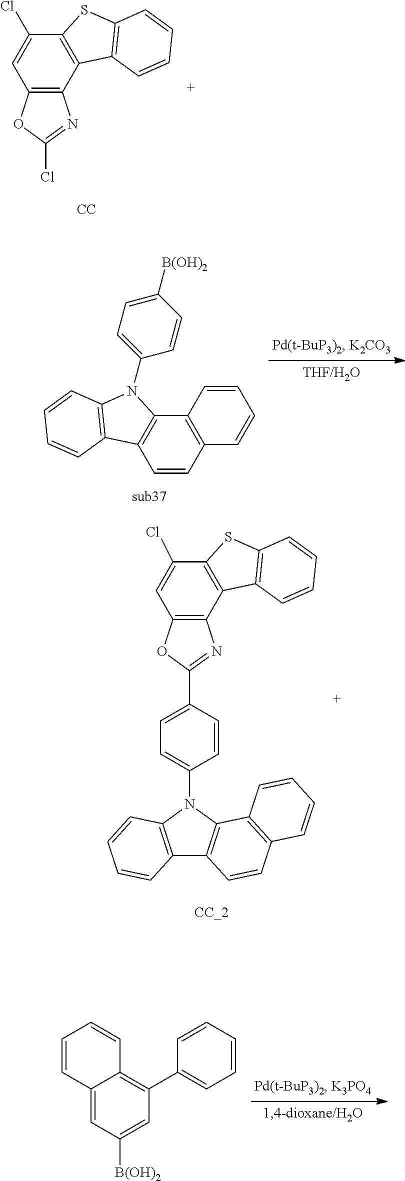

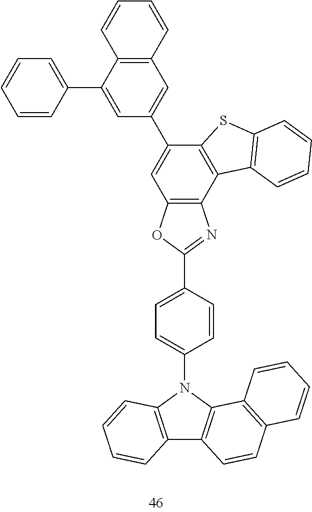 [SMILES: c1ccc(-c2cc(-c3cc4oc(-c5ccc(-n6c7ccccc7c7ccc8ccccc8c76)cc5)nc4c4c3sc3ccccc34)cc3ccccc23)cc1]

Compound CC (15 g, 51 mmol) and sub37 (18.1 g, 53.5 mmol) were added to THF (300 ml), and the mixture was stirred and refluxed. Then, potassium carbonate (21.1 g, 153 mmol) was dissolved in water (63 ml) and added thereto, and the mixture was sufficiently stirred, and then bis(tri-tert-butylphosphine)palladium(0) (0.3 g, 0.5 mmol) was added. After reacting for 2 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound CC_2 (21 g). (Yield: 75%, MS: [M+H]+=551)

Compound CC_2 (15 g, 27.2 mmol) and (4-phenylnaphthalen-2-yl)boronic acid (7.1 g, 28.6 mmol) were added to 1,4-dioxane (300 ml), and the mixture was stirred and refluxed. Then, potassium phosphate (17.3 g, 81.7 mmol) was dissolved in water (52 ml) and added thereto, and the mixture was sufficiently stirred, and then bis(tri-tert-butylphosphine)palladium(0) (0.1 g, 0.3 mmol) was added. After reacting for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 46 (12.9 g). (Yield: 66%, MS: [M+H]+=719)

### Example 47: Preparation of Compound 47

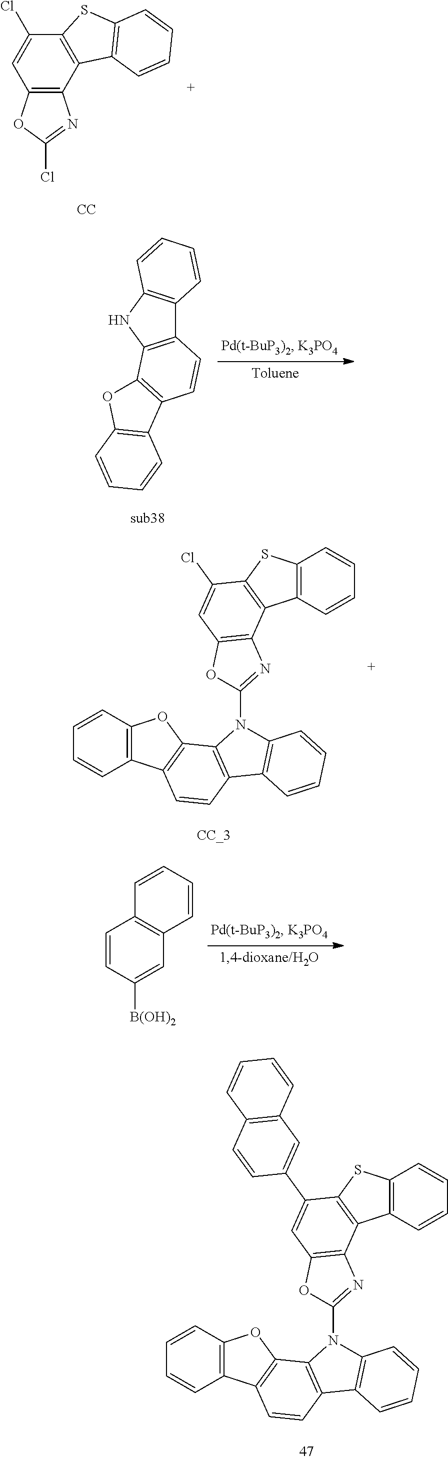

Compound CC (10 g, 34 mmol), sub38 (9.2 g, 35.7 mmol) and potassium phosphate (21.6 g, 102 mmol) were added to toluene (200 ml) under a nitrogen atmosphere, and the mixture was stirred and refluxed. Then, bis(tri-tert-butylphosphine)palladium(0) (0.2 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound CC_3 (11.9 g). (Yield: 68%, MS: [M+H]+=515)

Compound CC_3 (15 g, 29.1 mmol) and naphthalen-2-ylboronic acid (5.3 g, 30.6 mmol) were added to 1,4-dioxane (300 ml), and the mixture was stirred and refluxed. Then, potassium phosphate (18.5 g, 87.4 mmol) was dissolved in water (56 ml) and added thereto, and the mixture was sufficiently stirred, and then bis(tri-tert-butylphosphine)palladium(0) (0.1 g, 0.3 mmol) was added. After reacting for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 47 (13.1 g). (Yield: 74%, MS: [M+H]+=607)

### Example 48: Preparation of Compound 48

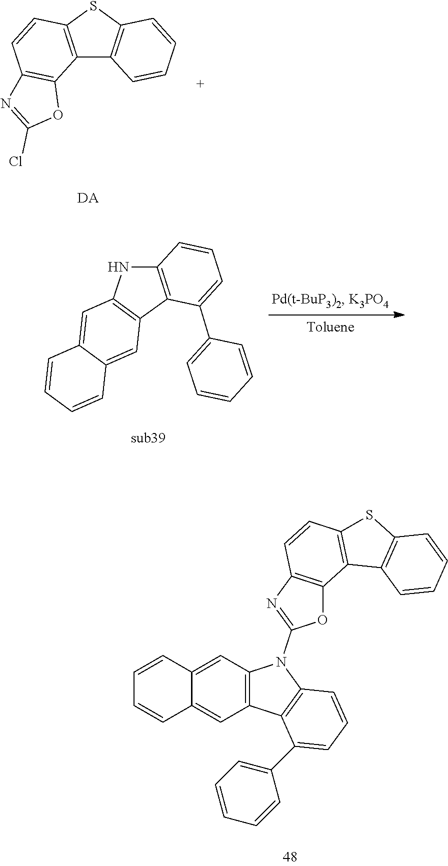 [SMILES: Clc1nc2ccc3sc4ccccc4c3c2o1 ; "DA" ; c1ccc(-c2cccc3[nH]c4cc5ccccc5cc4c23)cc1 ; c1ccc(-c2cccc3c2c2cc4ccccc4cc2n3-c2nc3ccc4sc5ccccc5c4c3o2)cc1]

Compound DA (10 g, 38.5 mmol), sub39 (11.9 g, 40.4 mmol) and potassium phosphate. (24.5 g, 115.5 mmol) were added to toluene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound 48 (12.5 g). (Yield: 63%, MS: [M+H]+=517)

### Example 49: Preparation of Compound 49

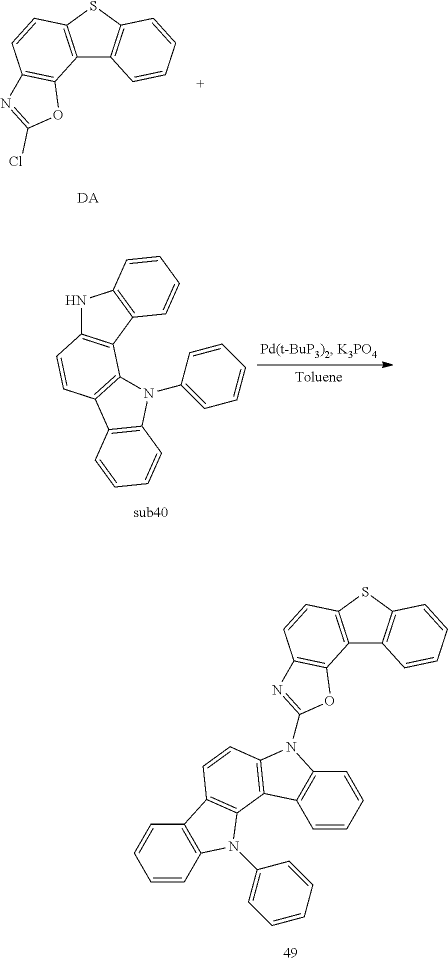 [SMILES: Clc1nc2ccc3sc4ccccc4c3c2o1 ; "DA" ; c1ccc(-n2c3ccccc3c3ccc4[nH]c5ccccc5c4c32)cc1 ; c1ccc(-n2c3ccccc3c3ccc4c(c5ccccc5n4-c4nc5ccc6sc7ccccc7c6c5o4)c32)cc1]

Compound DA (10 g, 38.5 mmol), sub40 (13.4 g, 40.4 mmol) and potassium phosphate (24.5 g, 115.5 mmol) were added to toluene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound 49 (13.9 g). (Yield: 65%, MS: [M+H]+=556)

### Example 50: Preparation of Compound 50

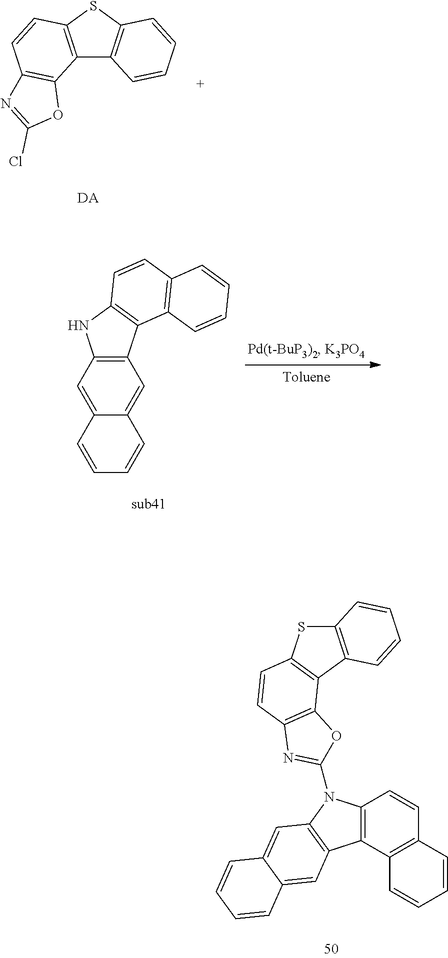 [SMILES: Clc1nc2ccc3sc4ccccc4c3c2o1 ; "DA" ; c1ccc2cc3c(cc2c1)[nH]c1ccc2ccccc2c13 ; c1ccc2cc3c(cc2c1)c1c2ccccc2ccc1n3-c1nc2ccc3sc4ccccc4c3c2o1]

Compound DA (10 g, 38.5 mmol), sub41 (10.8 g, 40.4 mmol) and potassium phosphate (24.5 g, 115.5 mmol) were added to toluene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound 50 (11.3 g). (Yield: 60%, MS: [M+H]+=491)

### Example 51: Preparation of Compound 51

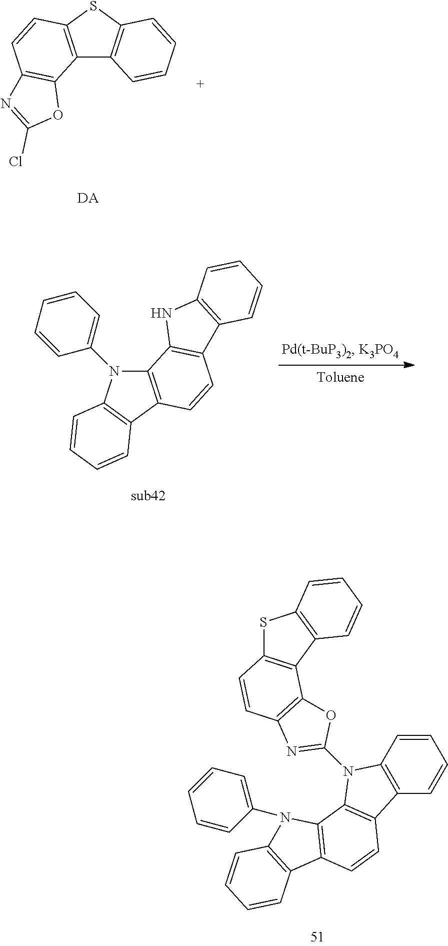 [SMILES: Clc1nc2ccc3sc4ccccc4c3c2o1 ; "DA" ; c1ccc(-n2c3ccccc3c3ccc4c5ccccc5[nH]c4c32)cc1 ; c1ccc(-n2c3ccccc3c3ccc4c5ccccc5n(-c5nc6ccc7sc8ccccc8c7c6o5)c4c32)cc1]

Compound DA (10 g, 38.5 mmol), sub42 (13.4 g, 40.4 mmol) and potassium phosphate (24.5 g, 115.5 mmol) were added to toluene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound 51 (15.2 g). (Yield: 71%, MS: [M+H]+=556)

### Example 52: Preparation of Compound 52

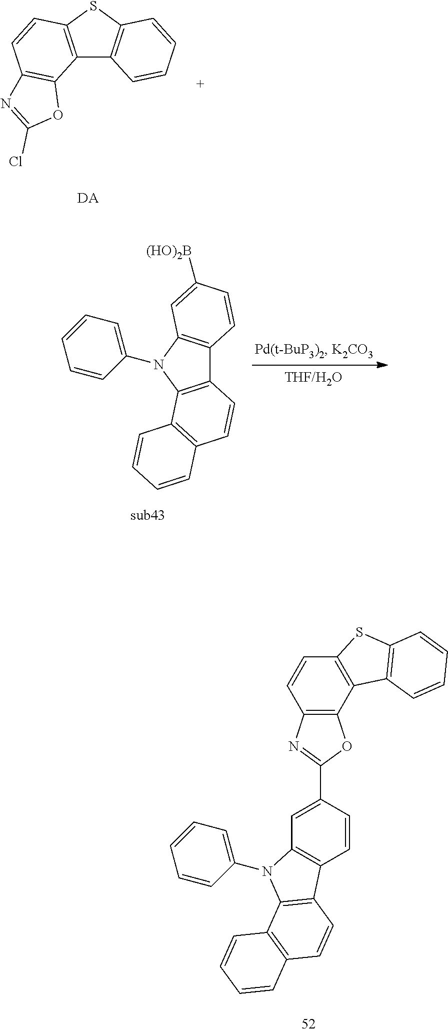 [SMILES: Clc1nc2ccc3sc4ccccc4c3c2o1 ; "DA" ; OB(O)c1ccc2c3ccc4ccccc4c3n(-c3ccccc3)c2c1 ; c1ccc(-n2c3cc(-c4nc5ccc6sc7ccccc7c6c5o4)ccc3c3ccc4ccccc4c32)cc1]

Compound DA (15 g, 57.8 mmol) and sub43 (20.4 g, 60.6 mmol) were added to THF (300 ml), and the mixture was stirred and refluxed. Then, potassium carbonate (23.9 g, 173.3 mmol) was dissolved in water (72 ml) and added thereto, and the mixture was sufficiently stirred, and then bis(tri-tert-butylphosphine)palladium(0) (0.3 g, 0.6 mmol) was added. After reacting for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 52 (21.2 g). (Yield: 71%, MS: [M+H]+=517)

### Example 53: Preparation of Compound 53

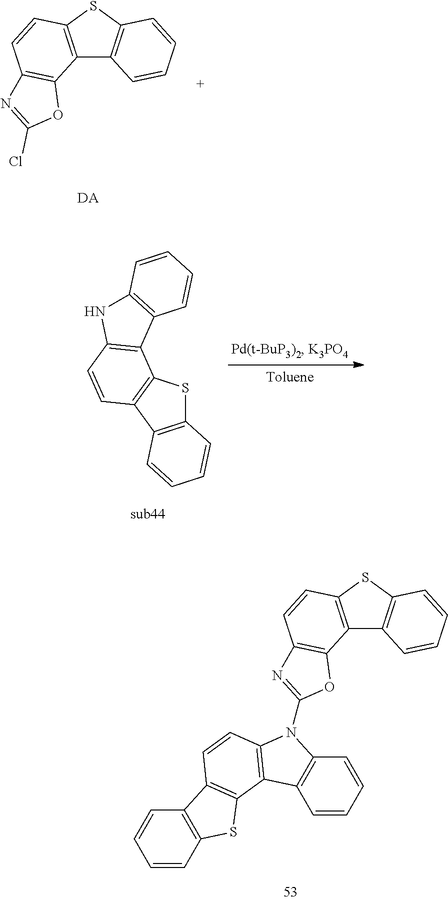 [SMILES: Clc1nc2ccc3sc4ccccc4c3c2o1 ; "DA" ; c1ccc2c(c1)[nH]c1ccc3c4ccccc4sc3c12 ; c1ccc2c(c1)sc1c2ccc2c1c1ccccc1n2-c1nc2ccc3sc4ccccc4c3c2o1]

Compound DA (10 g, 38.5 mmol), sub44 (11.1 g, 40.4 mmol) and potassium phosphate (24.5 g, 115.5 mmol) were added to toluene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound 53 (13.8 g). (Yield: 72%, MS: [M+H]+=497)

### Example 54: Preparation of Compound 54

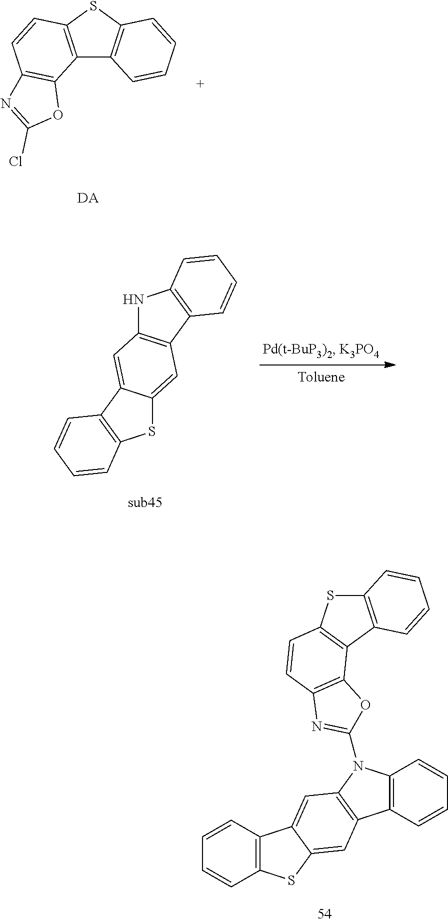 [SMILES: Clc1nc2ccc3sc4ccccc4c3c2o1 ; "DA" ; c1ccc2c(c1)[nH]c1cc3c(cc12)sc1ccccc13 ; c1ccc2c(c1)sc1cc3c4ccccc4n(-c4nc5ccc6sc7ccccc7c6c5o4)c3cc12]

Compound DA (10 g, 38.5 mmol), sub45 (11.1 g, 40.4 mmol) and potassium phosphate (24.5 g, 115.5 mmol) were added to toluene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound 54 (12.6 g). (Yield: 66%, MS: [M+H]+=497)

### Example 55: Preparation of Compound 55

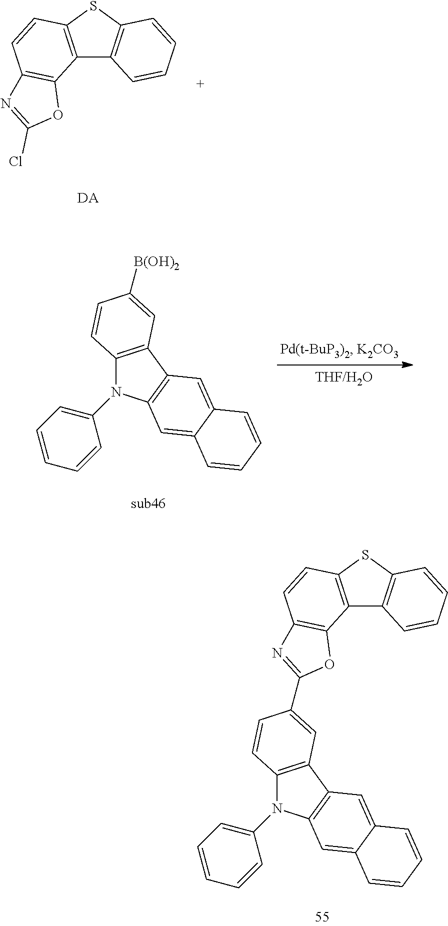 [SMILES: Clc1nc2ccc3sc4ccccc4c3c2o1 ; "DA" ; OB(O)c1ccc2c(c1)c1cc3ccccc3cc1n2-c1ccccc1 ; c1ccc(-n2c3ccc(-c4nc5ccc6sc7ccccc7c6c5o4)cc3c3cc4ccccc4cc32)cc1]

Compound DA (15 g, 57.8 mmol) and sub46 (20.4 g, 60.6 mmol) were added to THF (300 ml), and the mixture was stirred and refluxed. Then, potassium carbonate (23.9 g, 173.3 mmol) was dissolved in water (72 ml) and added thereto, and the mixture was sufficiently stirred, and then bis(tri-tert-butylphosphine)palladium(0) (0.3 g, 0.6 mmol) was added. After reacting for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 55 (22.1 g). (Yield: 74%, MS: [M+H]+=517)

### Example 56: Preparation of Compound 56

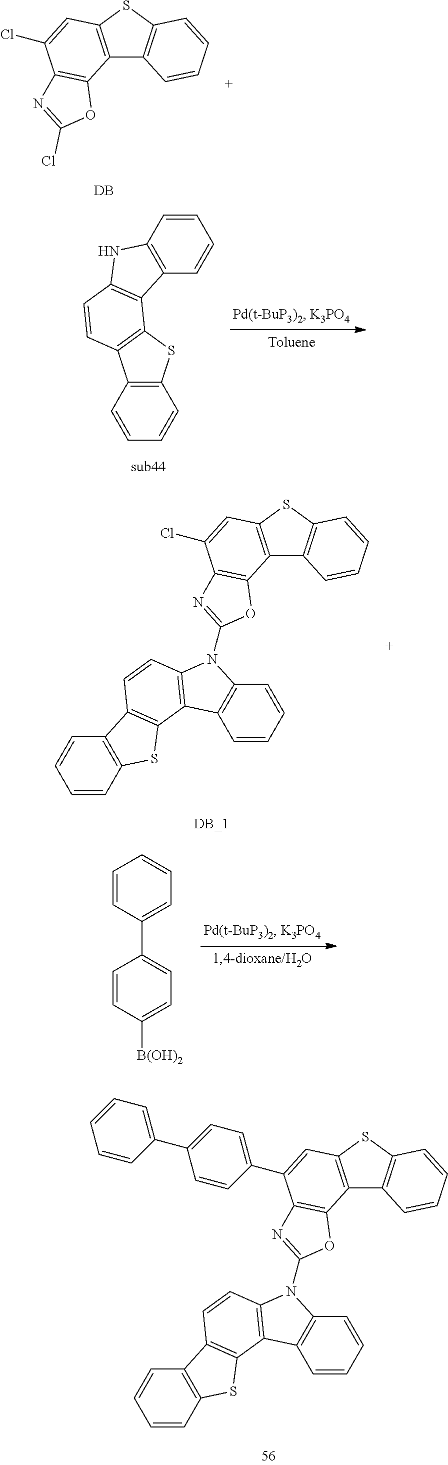

Compound DB (10 g, 34 mmol), sub44 (9.8 g, 35.7 mmol) and potassium phosphate (21.6 g, 102 mmol) were added to toluene (200 ml) under a nitrogen atmosphere, and the mixture was stirred and refluxed. Then, bis(tri-tert-butylphosphine)palladium(0) (0.2 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound DB_1 (11.7 g). (Yield: 65%, MS: [M+H]+=531)

Compound DB_1 (15 g, 28.2 mmol) and [1,1′-biphenyl]-4-ylboronic acid (5.9 g, 29.7 mmol) were added to 1,4-dioxane (300 ml), and the mixture was stirred and refluxed. Then, potassium phosphate (18 g, 84.7 mmol) was dissolved in water (54 ml) and added thereto, and the mixture was sufficiently stirred, and then bis(tri-tert-butylphosphine)palladium(0) (0.1 g, 0.3 mmol) was added. After reacting for 2 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 56 (12.1 g). (Yield: 66%, MS: [M+H]+=649)

### Example 57: Preparation of Compound 57

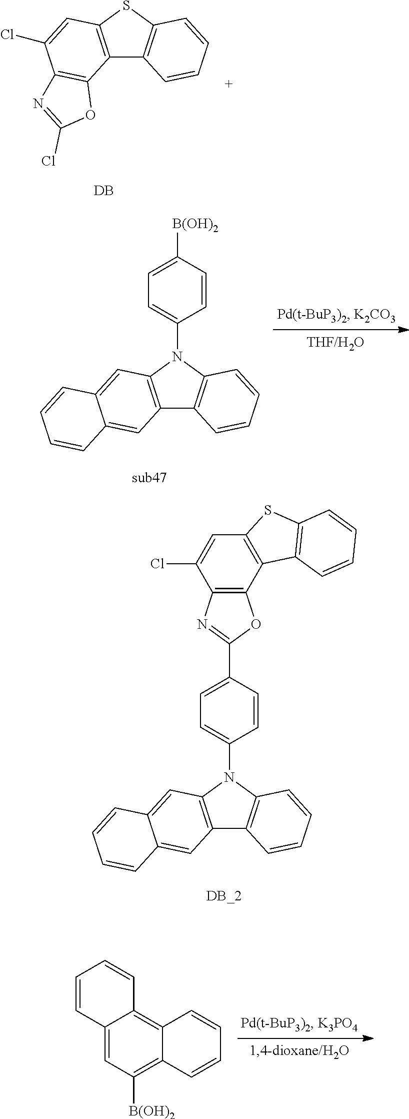

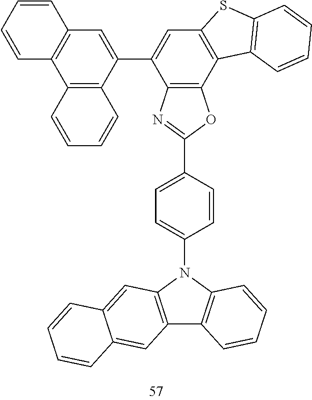 [SMILES: c1ccc2cc3c(cc2c1)c1ccccc1n3-c1ccc(-c2nc3c(-c4cc5ccccc5c5ccccc45)cc4sc5ccccc5c4c3o2)cc1]

Compound DB (15 g, 51 mmol) and sub47 (18.1 g, 53.5 mmol) were added to THF (300 ml), and the mixture was stirred and refluxed. Then, potassium carbonate (21.1 g, 153 mmol) was dissolved in water (63 ml) and added thereto, and the mixture was sufficiently stirred, and then bis(tri-tert-butylphosphine)palladium(0) (0.3 g, 0.5 mmol) was added. After reacting for 2 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound DB_2 (20.8 g). (Yield: 74%, MS: [M+H]+=551)

Compound DB_2 (15 g, 27.2 mmol) and phenanthren-9-ylboronic acid (6.4 g, 28.6 mmol) were added to 1,4-dioxane (300 ml), and the mixture was stirred and refluxed. Then, potassium phosphate (17.3 g, 81.7 mmol) was dissolved in water (52 ml) and added thereto, and the mixture was sufficiently stirred, and then bis(tri-tert-butylphosphine)palladium(0) (0.1 g, 0.3 mmol) was added. After reacting for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 57 (13.4 g). (Yield: 71%, MS: [M+H]+=693)

### Example 58: Preparation of Compound 58

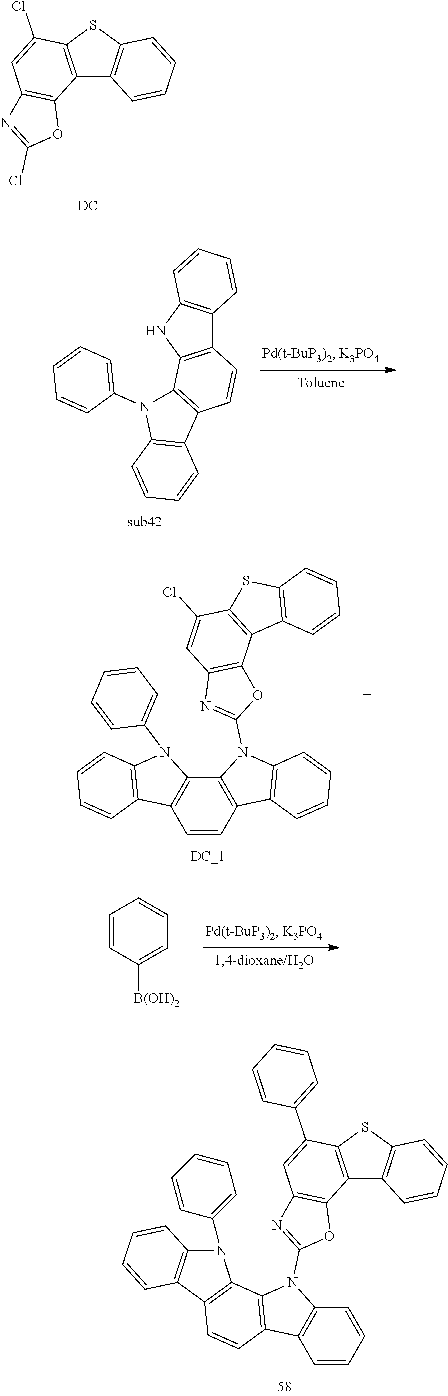 [SMILES: Clc1nc2cc(Cl)c3sc4ccccc4c3c2o1 ; "DC" ; c1ccc(-n2c3ccccc3c3ccc4c5ccccc5[nH]c4c32)cc1 ; Clc1cc2nc(-n3c4ccccc4c4ccc5c6ccccc6n(-c6ccccc6)c5c43)oc2c2c1sc1ccccc12 ; OB(O)c1ccccc1 ; c1ccc(-c2cc3nc(-n4c5ccccc5c5ccc6c7ccccc7n(-c7ccccc7)c6c54)oc3c3c2sc2ccccc23)cc1]

Compound DC (10 g, 34 mmol), sub42 (11.9 g, 35.7 mmol) and potassium phosphate (21.6 g, 102 mmol) were added to toluene (200 ml) under a nitrogen atmosphere, and the mixture was stirred and refluxed. Then, bis(tri-tert-butylphosphine)palladium(0) (0.2 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound DC_1 (13 g). (Yield: 65%, MS: [M+H]+=590)

Compound DC_1 (15 g, 25.4 mmol) and phenylboronic acid (3.3 g, 26.7 mmol) were added to 1,4-dioxane (300 ml), and the mixture was stirred and refluxed. Then, potassium phosphate (16.2 g, 76.3 mmol) was dissolved in water (49 ml) and added thereto, and the mixture was sufficiently stirred, and then bis(tri-tert-butylphosphine)palladium(0) (0.1 g, 0.3 mmol) was added. After reacting for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 58 (11.6 g). (Yield: 72%, MS: [M+H]+=632)

### Example 59: Preparation of Compound 59

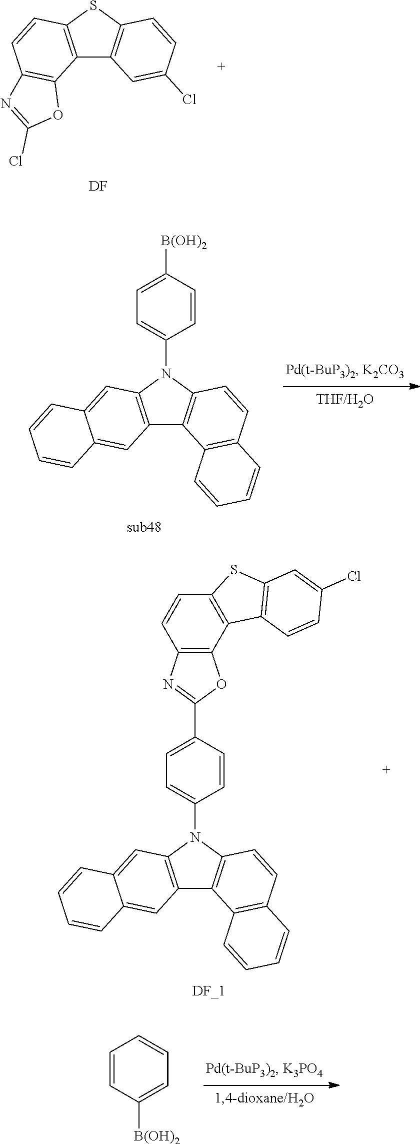 [SMILES: Clc1ccc2sc3ccc4nc(Cl)oc4c3c2c1 ; "DF" ; OB(O)c1ccc(-n2c3cc4ccccc4cc3c3c4ccccc4ccc32)cc1 ; Clc1ccc2c(c1)sc1ccc3nc(-c4ccc(-n5c6cc7ccccc7cc6c6c7ccccc7ccc65)cc4)oc3c12 ; OB(O)c1ccccc1]

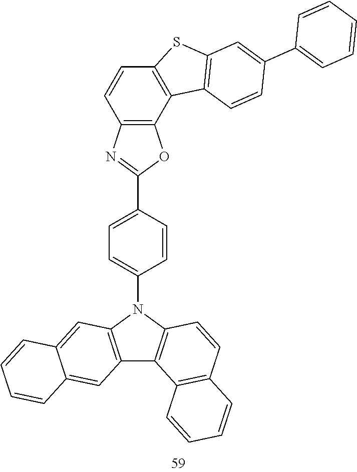 [SMILES: c1ccc(-c2ccc3c(c2)sc2ccc4nc(-c5ccc(-n6c7cc8ccccc8cc7c7c8ccccc8ccc76)cc5)oc4c23)cc1]

Compound DF (15 g, 51 mmol) and sub48 (20.7 g, 53.5 mmol) were added to THF (300 ml), and the mixture was stirred and refluxed. Then, potassium carbonate (21.1 g, 153 mmol) was dissolved in water (63 ml) and added thereto, and the mixture was sufficiently stirred, and then bis(tri-tert-butylphosphine)palladium(0) (0.3 g, 0.5 mmol) was added. After reacting for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound DF_1 (20.2 g). (Yield: 66%, MS: [M+H]+=601)

Compound DF_1 (15 g, 25 mmol) and phenylboronic acid (3.2 g, 26.2 mmol) were added to 1,4-dioxane (300 ml), and the mixture was stirred and refluxed. Then, potassium phosphate (15.9 g, 74.9 mmol) was dissolved in water (48 ml) and added thereto, and the mixture was sufficiently stirred, and then bis(tri-tert-butylphosphine)palladium(0) (0.1 g, 0.2 mmol) was added. After reacting for 2 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 59 (11.4 g). (Yield: 71%, MS: [M+H]+=643)

### Example 60: Preparation of Compound 60

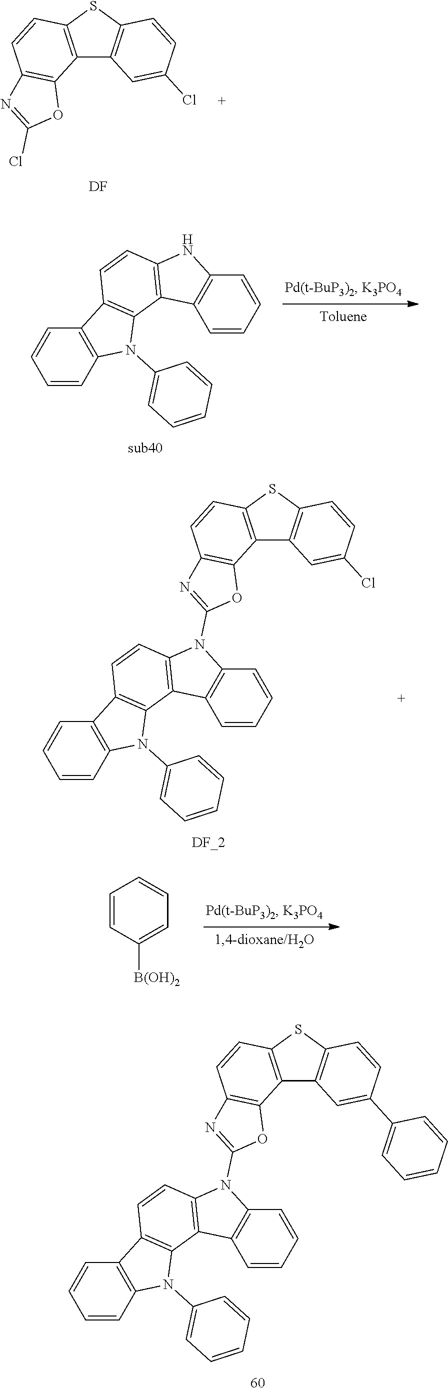 [SMILES: Clc1ccc2sc3ccc4nc(Cl)oc4c3c2c1 ; "DF" ; c1ccc(-n2c3ccccc3c3ccc4[nH]c5ccccc5c4c32)cc1 ; Clc1ccc2sc3ccc4nc(-n5c6ccccc6c6c5ccc5c7ccccc7n(-c7ccccc7)c56)oc4c3c2c1 ; OB(O)c1ccccc1 ; c1ccc(-c2ccc3sc4ccc5nc(-n6c7ccccc7c7c6ccc6c8ccccc8n(-c8ccccc8)c67)oc5c4c3c2)cc1]

Compound DF (10 g, 34 mmol), sub40 (11.9 g, 35.7 mmol) and potassium phosphate (21.6 g, 102 mmol) were added to toluene (200 ml) under a nitrogen atmosphere, and the mixture was stirred and refluxed. Then, bis(tri-tert-butylphosphine)palladium(0) (0.2 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound DF_2 (13.8 g). (Yield: 69%, MS: [M+H]+=590)

Compound DF_2 (15 g, 25 mmol) and phenylboronic acid (3.2 g, 26.2 mmol) were added to 1,4-dioxane (300 ml), and the mixture was stirred and refluxed. Then, potassium phosphate (15.9 g, 74.9 mmol) was dissolved in water (48 ml) and added thereto, and the mixture was sufficiently stirred, and then bis(tri-tert-butylphosphine)palladium(0) (0.1 g, 0.2 mmol) was added. After reacting for 2 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 60 (11.4 g). (Yield: 71%, MS: [M+H]+=643)

### Example 61: Preparation of Compound 61

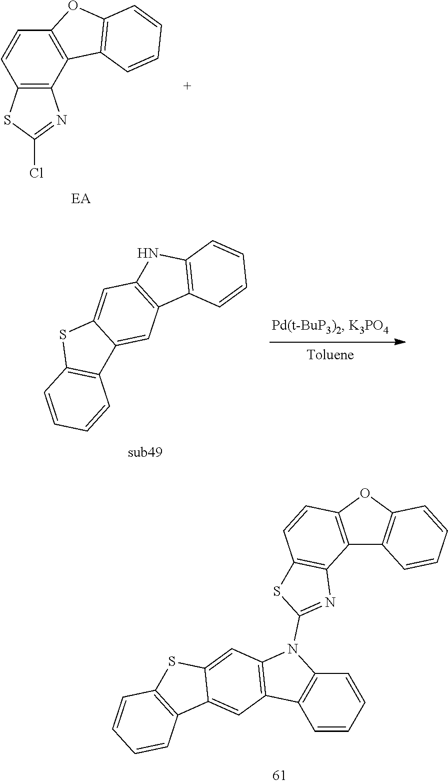 [SMILES: Clc1nc2c(ccc3oc4ccccc4c32)s1 ; c1ccc2c(c1)[nH]c1cc3sc4ccccc4c3cc12 ; c1ccc2c(c1)oc1ccc3sc(-n4c5ccccc5c5cc6c(cc54)sc4ccccc46)nc3c12]

Compound EA (10 g, 38.5 mmol), sub49 (11.1 g, 40.4 mmol) and potassium phosphate (24.5 g, 115.5 mmol) were added to toluene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound 61 (11.7 g). (Yield: 61%, MS: [M+H]+=497)

### Example 62: Preparation of Compound 62

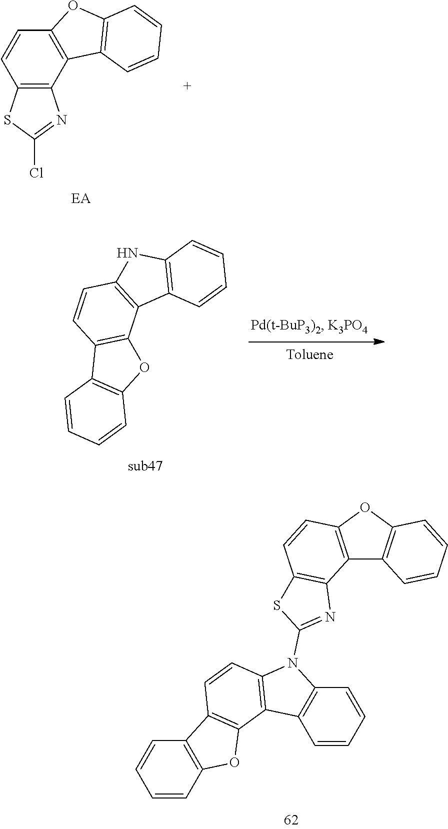 [SMILES: Clc1nc2c(ccc3oc4ccccc4c32)s1 ; c1ccc2c(c1)[nH]c1ccc3c4ccccc4oc3c12 ; c1ccc2c(c1)oc1c2ccc2c1c1ccccc1n2-c1nc2c(ccc3oc4ccccc4c32)s1]

Compound EA (10 g, 38.5 mmol), sub47 (10.4 g, 40.4 mmol) and potassium phosphate (24.5 g, 115.5 mmol) were added to toluene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound 62 (12.4 g). (Yield: 67%, MS: [M+H]+=481)

### Example 63: Preparation of Compound 63

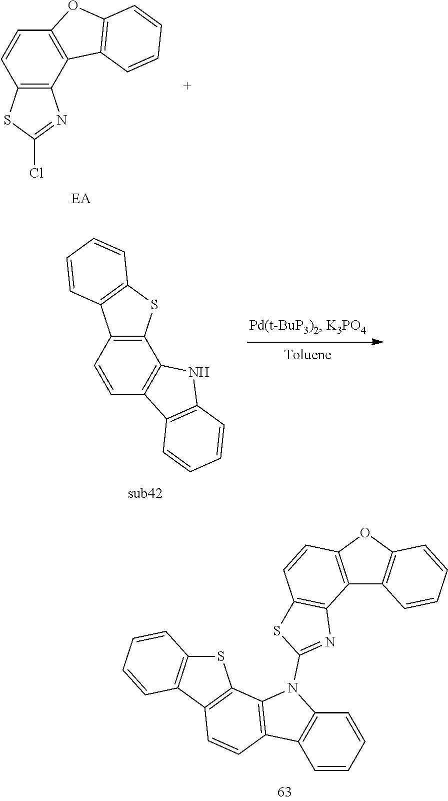 [SMILES: Clc1nc2c(ccc3oc4ccccc4c32)s1 ; c1ccc2c(c1)[nH]c1c2ccc2c3ccccc3sc21 ; c1ccc2c(c1)oc1ccc3sc(-n4c5ccccc5c5ccc6c7ccccc7sc6c54)nc3c12]

Compound EA (10 g, 38.5 mmol), sub42 (11.1 g, 40.4 mmol) and potassium phosphate (24.5 g, 115.5 mmol) were added to toluene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound 63 (11.7 g). (Yield: 61%, MS: [M+H]+=497)

### Example 64: Preparation of Compound 64

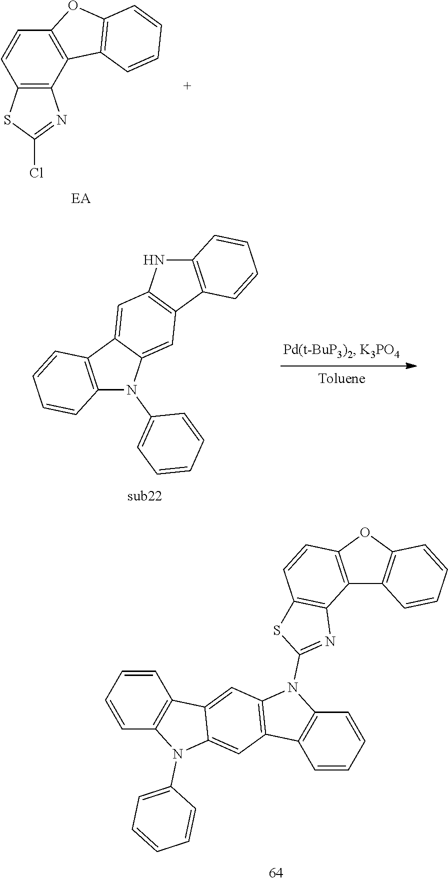 [SMILES: Clc1nc2c(ccc3oc4ccccc4c32)s1 ; c1ccc(-n2c3ccccc3c3cc4[nH]c5ccccc5c4cc32)cc1 ; c1ccc(-n2c3ccccc3c3cc4c(cc32)c2ccccc2n4-c2nc3c(ccc4oc5ccccc5c43)s2)cc1]

Compound EA (10 g, 38.5 mmol), sub22 (13.4 g, 40.4 mmol) and potassium phosphate (24.5 g, 115.5 mmol) were added to toluene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound 64 (13 g). (Yield: 61%, MS: [M+H]+=556)

### Example 65: Preparation of Compound 65

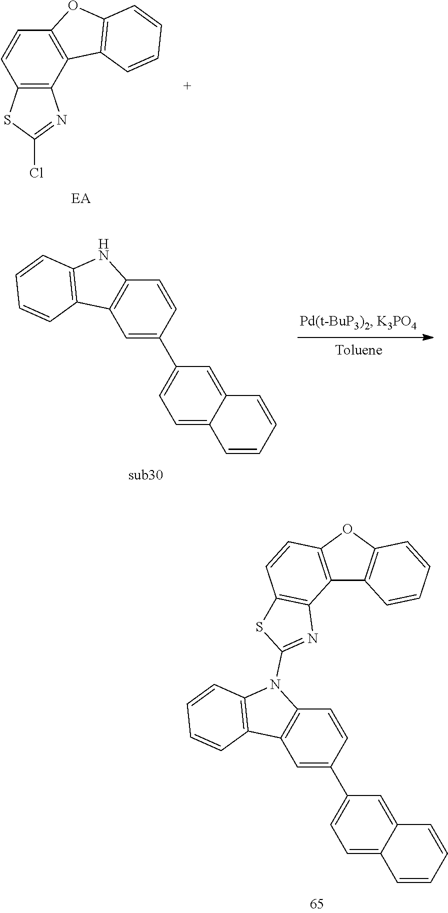 [SMILES: Clc1nc2c(ccc3oc4ccccc4c32)s1 ; c1ccc2cc(-c3ccc4[nH]c5ccccc5c4c3)ccc2c1 ; c1ccc2cc(-c3ccc4c(c3)c3ccccc3n4-c3nc4c(ccc5oc6ccccc6c54)s3)ccc2c1]

Compound EA (10 g, 38.5 mmol), sub30 (13.4 g, 40.4 mmol) and potassium phosphate (24.5 g, 115.5 mmol) were added to toluene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound 65 (16 g). (Yield: 75%, MS: [M+H]+=517)

### Example 66: Preparation of Compound 66

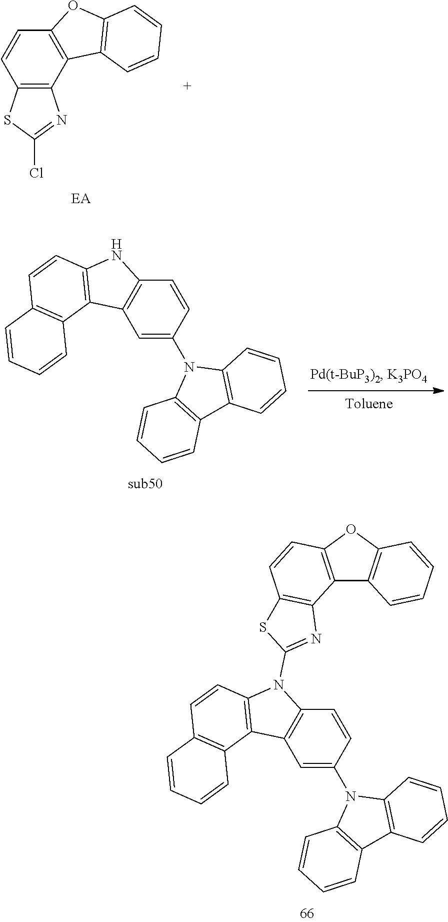 [SMILES: Clc1nc2c(ccc3oc4ccccc4c32)s1 ; c1ccc2c(c1)ccc1[nH]c3ccc(-n4c5ccccc5c5ccccc54)cc3c12 ; c1ccc2c(c1)ccc1c2c2cc(-n3c4ccccc4c4ccccc43)ccc2n1-c1nc2c(ccc3oc4ccccc4c32)s1]

Compound EA (10 g, 38.5 mmol), sub50 (15.5 g, 40.4 mmol) and potassium phosphate (24.5 g, 115.5 mmol) were added to toluene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound 66 (16.3 g). (Yield: 70%, MS: [M+H]+=606)

### Example 67: Preparation of Compound 67

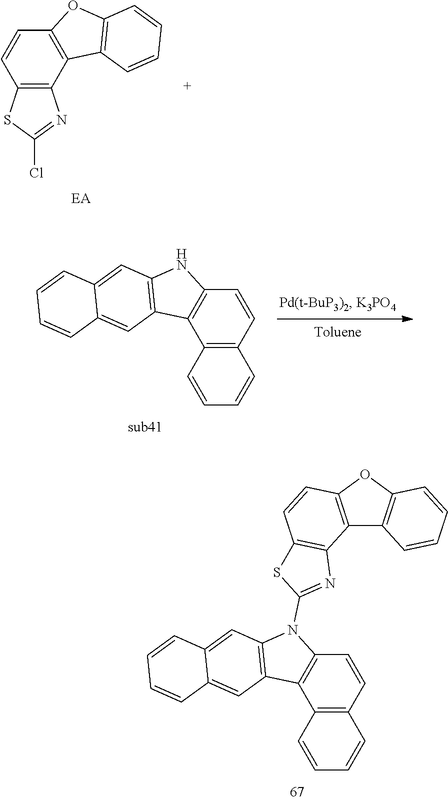 [SMILES: Clc1nc2c(ccc3oc4ccccc4c32)s1 ; c1ccc2cc3c(cc2c1)[nH]c1ccc2ccccc2c13 ; c1ccc2cc3c(cc2c1)c1c2ccccc2ccc1n3-c1nc2c(ccc3oc4ccccc4c32)s1]

Compound EA (10 g, 38.5 mmol), sub41 (10.8 g, 40.4 mmol) and potassium phosphate (24.5 g, 115.5 mmol) were added to toluene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound 67 (13 g). (Yield: 69%, MS: [M+H]+=491)

### Example 68: Preparation of Compound 68

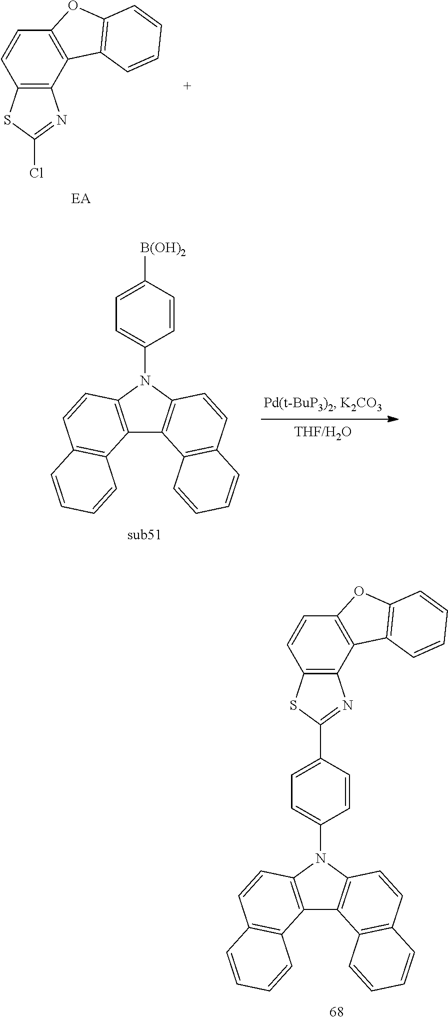 [SMILES: Clc1nc2c(ccc3oc4ccccc4c32)s1 ; OB(O)c1ccc(-n2c3ccc4ccccc4c3c3c4ccccc4ccc32)cc1 ; c1ccc2c(c1)ccc1c2c2c3ccccc3ccc2n1-c1ccc(-c2nc3c(ccc4oc5ccccc5c43)s2)cc1]

Compound EA (15 g, 57.8 mmol) and sub51 (23.5 g, 60.6 mmol) were added to THF (300 ml), and the mixture was stirred and refluxed. Then, potassium carbonate (23.9 g, 173.3 mmol) was dissolved in water (72 ml) and added thereto, and the mixture was sufficiently stirred, and then bis(tri-tert-butylphosphine)palladium(0) (0.3 g, 0.6 mmol) was added. After reacting for 2 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 68 (22.2 g). (Yield: 68%, MS: [M+H]+=567)

### Example 69: Preparation of Compound 69

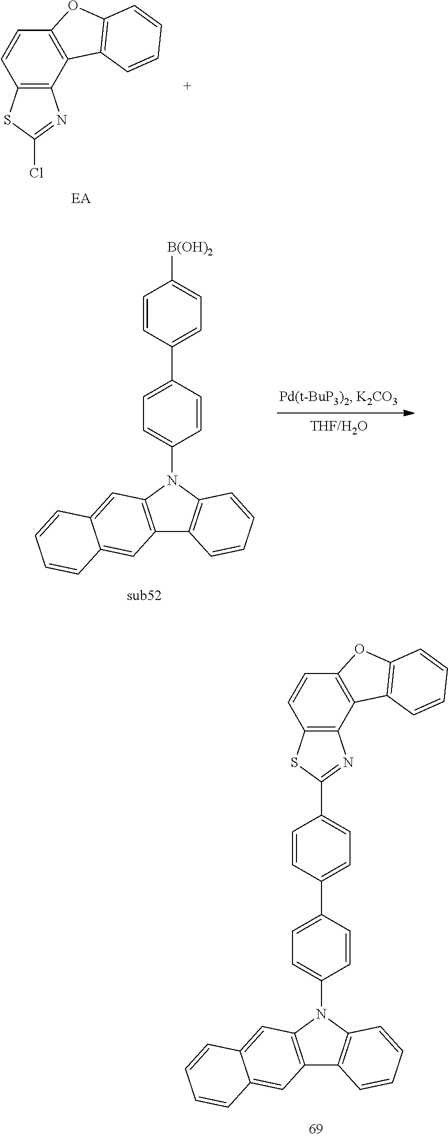 [SMILES: Clc1nc2c(ccc3oc4ccccc4c32)s1 ; OB(O)c1ccc(-c2ccc(-n3c4ccccc4c4cc5ccccc5cc43)cc2)cc1 ; c1ccc2cc3c(cc2c1)c1ccccc1n3-c1ccc(-c2ccc(-c3nc4c(ccc5oc6ccccc6c54)s3)cc2)cc1]

Compound EA (15 g, 57.8 mmol) and sub52 (25.1 g, 60.6 mmol) were added to THF (300 ml), and the mixture was stirred and refluxed. Then, potassium carbonate (23.9 g, 173.3 mmol) was dissolved in water (72 ml) and added thereto, and the mixture was sufficiently stirred, and then bis(tri-tert-butylphosphine)palladium(0) (0.3 g, 0.6 mmol) was added. After reacting for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 69 (24.6 g). (Yield: 72%, MS: [M+H]+=593)

### Example 70: Preparation of Compound 70

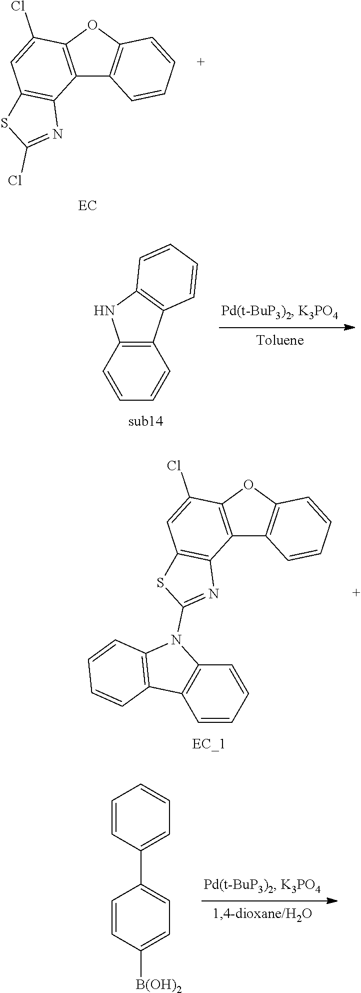 [SMILES: Clc1nc2c(cc(Cl)c3oc4ccccc4c32)s1 ; "EC" ; c1ccc2c(c1)[nH]c1ccccc12 ; Clc1cc2sc(-n3c4ccccc4c4ccccc43)nc2c2c1oc1ccccc12 ; OB(O)c1ccc(-c2ccccc2)cc1]

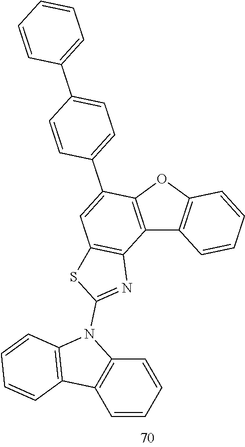 [SMILES: c1ccc(-c2ccc(-c3cc4sc(-n5c6ccccc6c6ccccc65)nc4c4c3oc3ccccc34)cc2)cc1]

Compound EC (10 g, 34 mmol), sub14 (6 g, 35.7 mmol) and potassium phosphate (21.6 g, 102 mmol) were added to toluene (200 ml) under a nitrogen atmosphere, and the mixture was stirred and refluxed. Then, bis(tri-tert-butylphosphine)palladium(0) (0.2 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound EC_1 (9.8 g). (Yield: 68%, MS: [M+H]+=425)

Compound EC_1 (15 g, 35.3 mmol) and [1,1′-biphenyl]-4-ylboronic acid (7.3 g, 37.1 mmol) were added to 1,4-dioxane (300 ml), and the mixture was stirred and refluxed. Then, potassium phosphate (22.5 g, 105.9 mmol) was dissolved in water (67 ml) and added thereto, and the mixture was sufficiently stirred, and then bis(tri-tert-butylphosphine)palladium(0) (0.2 g, 0.4 mmol) was added. After reacting for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 70 (11.5 g). (Yield: 60%, MS: [M+H]+=543)

### Example 71: Preparation of Compound 71

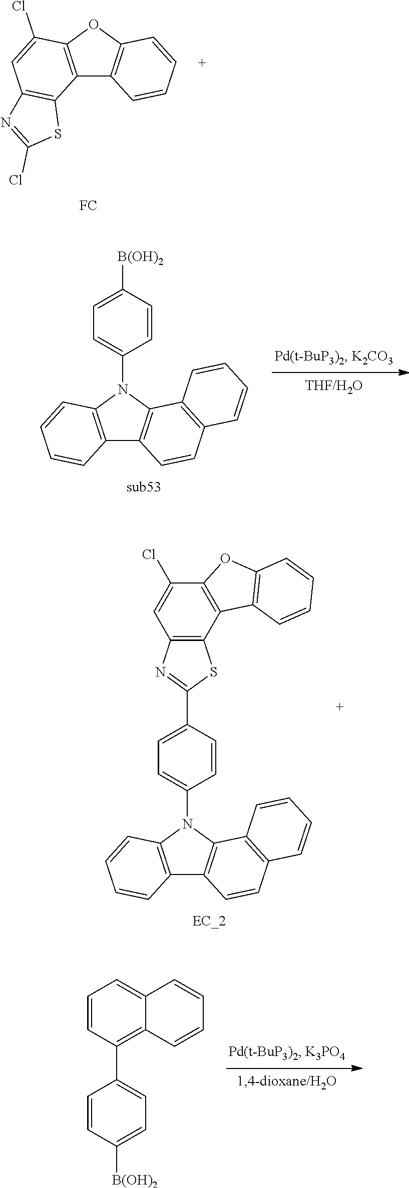 [SMILES: Clc1nc2cc(Cl)c3oc4ccccc4c3c2s1 ; "FC" ; OB(O)c1ccc(-n2c3ccccc3c3ccc4ccccc4c32)cc1 ; Clc1cc2nc(-c3ccc(-n4c5ccccc5c5ccc6ccccc6c54)cc3)sc2c2c1oc1ccccc12 ; OB(O)c1ccc(-c2cccc3ccccc23)cc1]

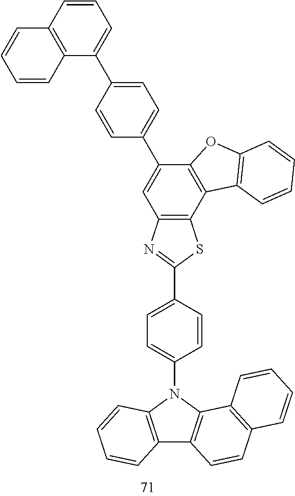 [SMILES: c1ccc2c(-c3ccc(-c4cc5nc(-c6ccc(-n7c8ccccc8c8ccc9ccccc9c87)cc6)sc5c5c4oc4ccccc45)cc3)cccc2c1]

Compound FC (15 g, 51 mmol) and sub53 (18.1 g, 53.5 mmol) were added to THF (300 ml), and the mixture was stirred and refluxed. Then, potassium carbonate (21.1 g, 153 mmol) was dissolved in water (63 ml) and added thereto, and the mixture was sufficiently stirred, and then bis(tri-tert-butylphosphine)palladium(0) (0.3 g, 0.5 mmol) was added. After reacting for 2 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound EC_2 (17.1 g). (Yield: 61%, MS: [M+H]+=551)

Compound EC_2 (15 g, 27.3 mmol) and (4-(naphthalen-1-yl)phenyl)boronic acid (7.1 g, 28.6 mmol) were added to 1,4-dioxane (300 ml), and the mixture was stirred and refluxed. Then, potassium phosphate (17.4 g, 81.8 mmol) was dissolved in water (52 ml) and added thereto, and the mixture was sufficiently stirred, and then bis(tri-tert-butylphosphine)palladium(0) (0.1 g, 0.3 mmol) was added. After reacting for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 71 (12.5 g). (Yield: 64%, MS: [M+H]+=719)

### Example 72: Preparation of Compound 72

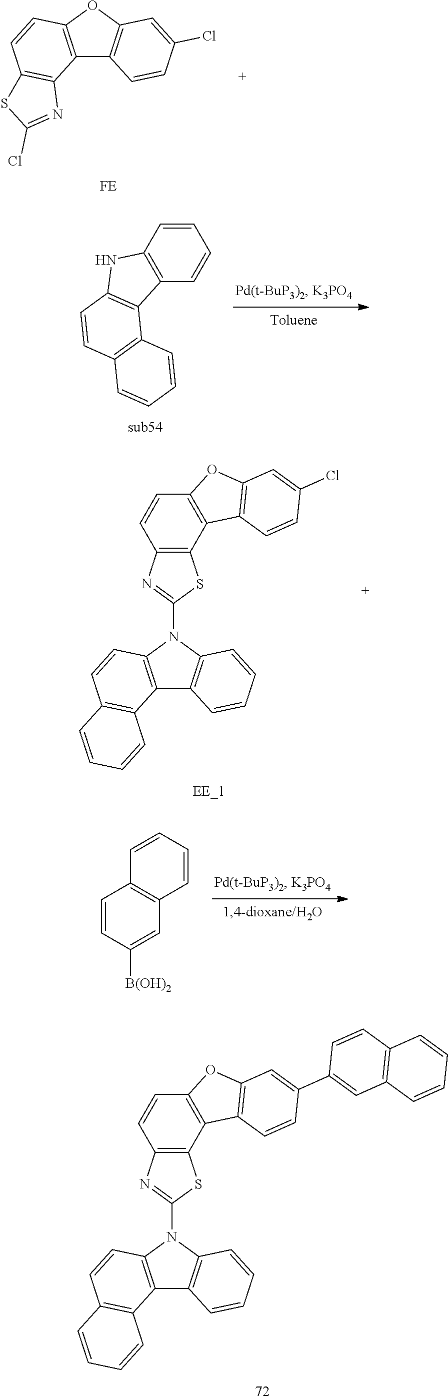 [SMILES: Clc1ccc2c(c1)oc1ccc3sc(Cl)nc3c12 ; "FE" ; c1ccc2c(c1)ccc1[nH]c3ccccc3c12 ; Clc1ccc2c(c1)oc1ccc3nc(-n4c5ccccc5c5c6ccccc6ccc54)sc3c12 ; OB(O)c1ccc2ccccc2c1 ; c1ccc2cc(-c3ccc4c(c3)oc3ccc5nc(-n6c7ccccc7c7c8ccccc8ccc76)sc5c34)ccc2c1]

Compound FE (10 g, 34 mmol), sub54 (7.8 g, 35.7 mmol) and potassium phosphate (21.6 g, 102 mmol) were added to toluene (200 ml) under a nitrogen atmosphere, and the mixture was stirred and refluxed. Then, bis(tri-tert-butylphosphine)palladium(0) (0.2 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound EE_1 (10.6 g). (Yield: 66%, MS: [M+H]+=475)

Compound EE_1 (15 g, 31.6 mmol) and naphthalen-2-ylboronic acid (5.7 g, 33.2 mmol) were added to 1,4-dioxane (300 ml), and the mixture was stirred and refluxed. Then, potassium phosphate (20.1 g, 94.7 mmol) was dissolved in water (60 ml) and added thereto, and the mixture was sufficiently stirred, and then bis(tri-tert-butylphosphine)palladium(0) (0.2 g, 0.3 mmol) was added. After reacting for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 72 (11.6 g). (Yield: 65%, MS: [M+H]+=567)

### Example 73: Preparation of Compound 73

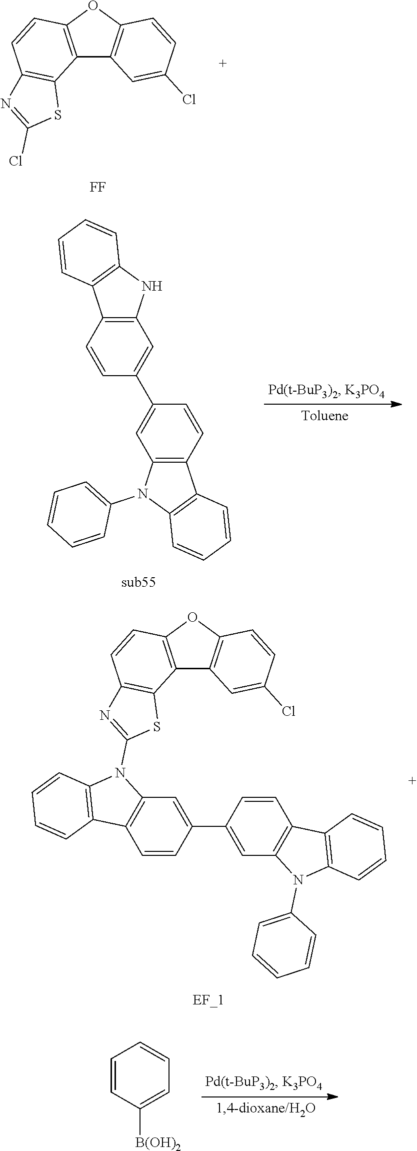

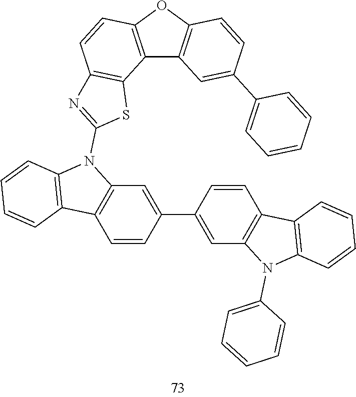 [SMILES: c1ccc(-c2ccc3oc4ccc5nc(-n6c7ccccc7c7ccc(-c8ccc9c%10ccccc%10n(-c%10ccccc%10)c9c8)cc76)sc5c4c3c2)cc1]

Compound FF (10 g, 34 mmol), sub55 (14.6 g, 35.7 mmol) and potassium phosphate (21.6 g, 102 mmol) were added to toluene (200 ml) under a nitrogen atmosphere, and the mixture was stirred and refluxed. Then, bis(tri-tert-butylphosphine)palladium(0) (0.2 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound EF_1 (14.5 g). (Yield: 64%, MS: [M+H]+=666)

Compound EF_1 (15 g, 22.5 mmol) and phenylboronic acid (2.9 g, 23.6 mmol) were added to 1,4-dioxane (300 ml), and the mixture was stirred and refluxed. Then, potassium phosphate (14.3 g, 67.5 mmol) was dissolved in water (43 ml) and added thereto, and the mixture was sufficiently stirred, and then bis(tri-tert-butylphosphine)palladium(0) (0.1 g, 0.2 mmol) was added. After reacting for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 73 (10.7 g). (Yield: 67%, MS: [M+H]+=708)

### Example 74: Preparation of Compound 74

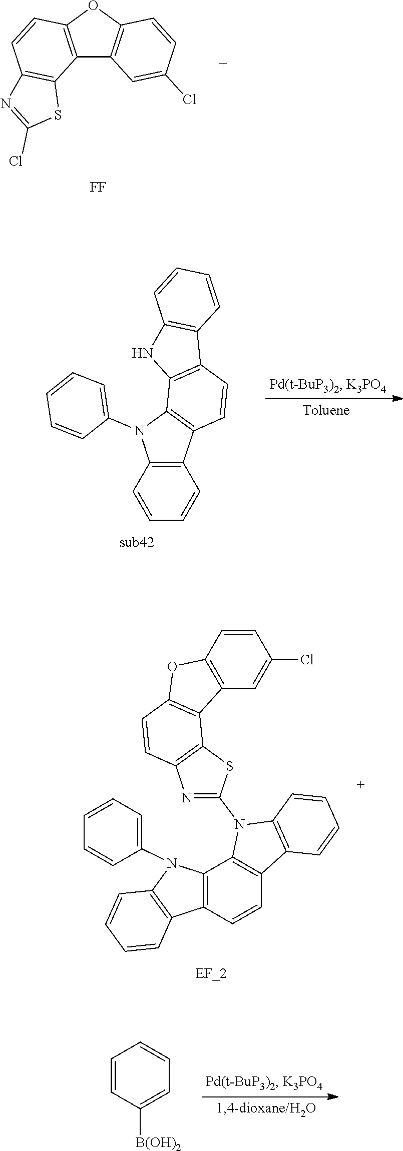 [SMILES: Clc1ccc2oc3ccc4nc(Cl)sc4c3c2c1 ; "FF" ; c1ccc(-n2c3ccccc3c3ccc4c5ccccc5[nH]c4c32)cc1 ; Clc1ccc2oc3ccc4nc(-n5c6ccccc6c6ccc7c8ccccc8n(-c8ccccc8)c7c65)sc4c3c2c1 ; OB(O)c1ccccc1]

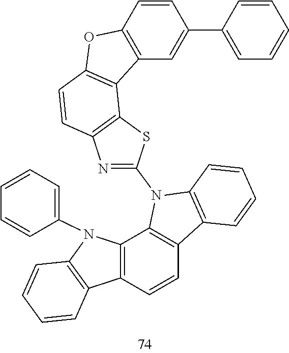 [SMILES: c1ccc(-c2ccc3oc4ccc5nc(-n6c7ccccc7c7ccc8c9ccccc9n(-c9ccccc9)c8c76)sc5c4c3c2)cc1]

Compound FF (10 g, 34 mmol), sub42 (11.9 g, 35.7 mmol) and potassium phosphate (21.6 g, 102 mmol) were added to toluene (200 ml) under a nitrogen atmosphere, and the mixture was stirred and refluxed. Then, bis(tri-tert-butylphosphine)palladium(0) (0.2 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound EF_2 (12.6 g). (Yield: 63%, MS: [M+H]+=590)

Compound EF_2 (15 g, 25.4 mmol) and phenylboronic acid (3.3 g, 26.7 mmol) were added to 1,4-dioxane (300 ml), and the mixture was stirred and refluxed. Then, potassium phosphate (16.2 g, 76.3 mmol) was dissolved in water (49 ml) and added thereto, and the mixture was sufficiently stirred, and then bis(tri-tert-butylphosphine)palladium(0) (0.1 g, 0.3 mmol) was added. After reacting for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 74 (10.9 g). (Yield: 68%, MS: [M+H]+=632)

### Example 75: Preparation of Compound 75

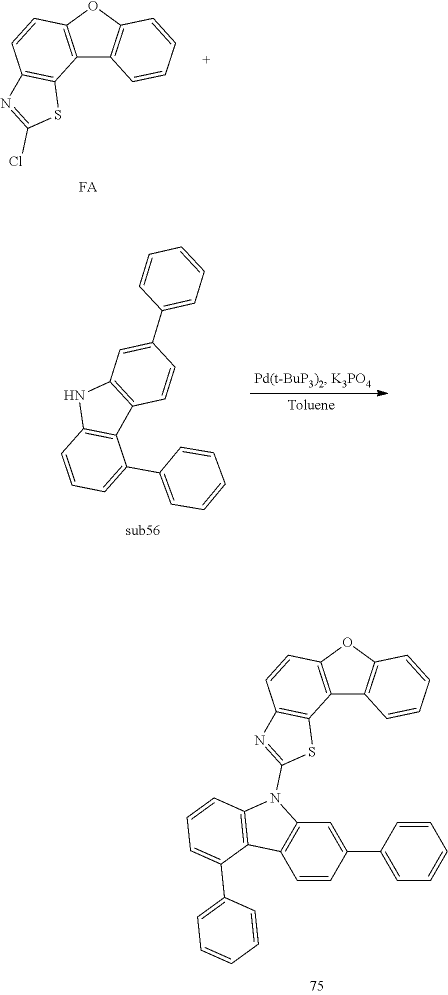 [SMILES: Clc1nc2ccc3oc4ccccc4c3c2s1 ; "FA" ; c1ccc(-c2ccc3c(c2)[nH]c2cccc(-c4ccccc4)c23)cc1 ; c1ccc(-c2ccc3c4c(-c5ccccc5)cccc4n(-c4nc5ccc6oc7ccccc7c6c5s4)c3c2)cc1]

Compound FA (10 g, 38.5 mmol), sub56 (12.9 g, 40.4 mmol) and potassium phosphate (24.5 g, 115.5 mmol) were added to toluene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound 75 (15.4 g). (Yield: 74%, MS: [M+H]+=543)

### Example 76: Preparation of Compound 76

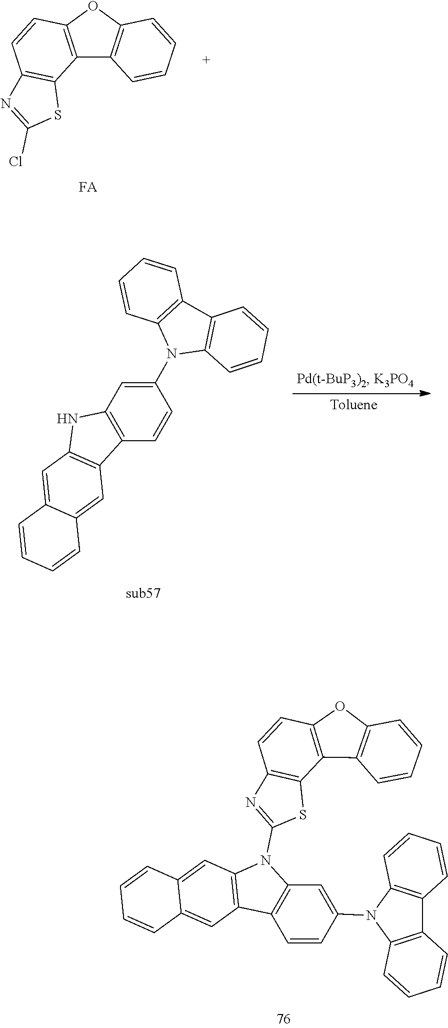 [SMILES: Clc1nc2ccc3oc4ccccc4c3c2s1 ; "FA" ; c1ccc2cc3c(cc2c1)[nH]c1cc(-n2c4ccccc4c4ccccc42)ccc13 ; c1ccc2cc3c(cc2c1)c1ccc(-n2c4ccccc4c4ccccc42)cc1n3-c1nc2ccc3oc4ccccc4c3c2s1]

Compound FA (10 g, 38.5 mmol), sub57 (15.5 g, 40.4 mmol) and potassium phosphate (24.5 g, 115.5 mmol) were added to toluene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound 76 (15.4 g). (Yield: 66%, MS: [M+H]+=606)

### Example 77: Preparation of Compound 77

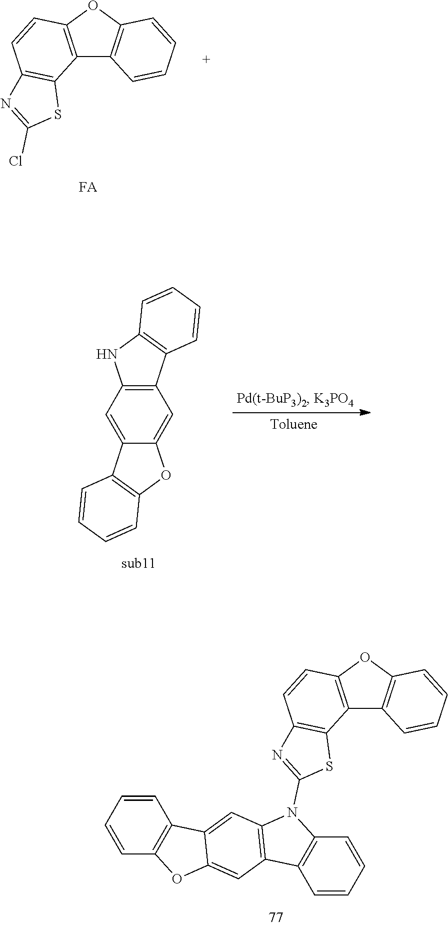 [SMILES: Clc1nc2ccc3oc4ccccc4c3c2s1 ; "FA" ; c1ccc2c(c1)[nH]c1cc3c(cc12)oc1ccccc13 ; c1ccc2c(c1)oc1cc3c4ccccc4n(-c4nc5ccc6oc7ccccc7c6c5s4)c3cc12]

Compound FA (10 g, 38.5 mmol), sub11 (10.4 g, 40.4 mmol) and potassium phosphate (24.5 g, 115.5 mmol) were added to toluene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound 77 (11.3 g). (Yield: 61%, MS: [M+H]+=481)

### Example 78: Preparation of Compound 78

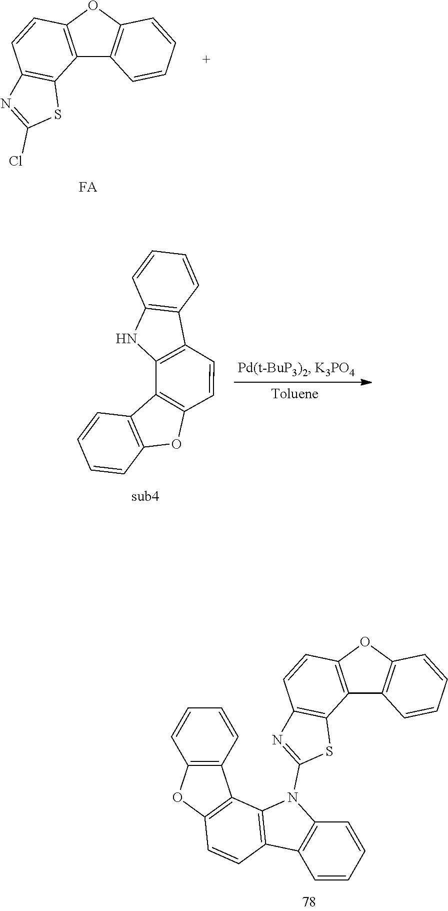 [SMILES: Clc1nc2ccc3oc4ccccc4c3c2s1 ; "FA" ; c1ccc2c(c1)[nH]c1c2ccc2oc3ccccc3c21 ; c1ccc2c(c1)oc1ccc3nc(-n4c5ccccc5c5ccc6oc7ccccc7c6c54)sc3c12]

Compound FA (10 g, 38.5 mmol), sub4 (10.4 g, 40.4 mmol) and potassium phosphate (24.5 g, 115.5 mmol) were added to toluene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound 78 (11.5 g). (Yield: 62%, MS: [M+H]+=481)

### Example 79: Preparation of Compound 79

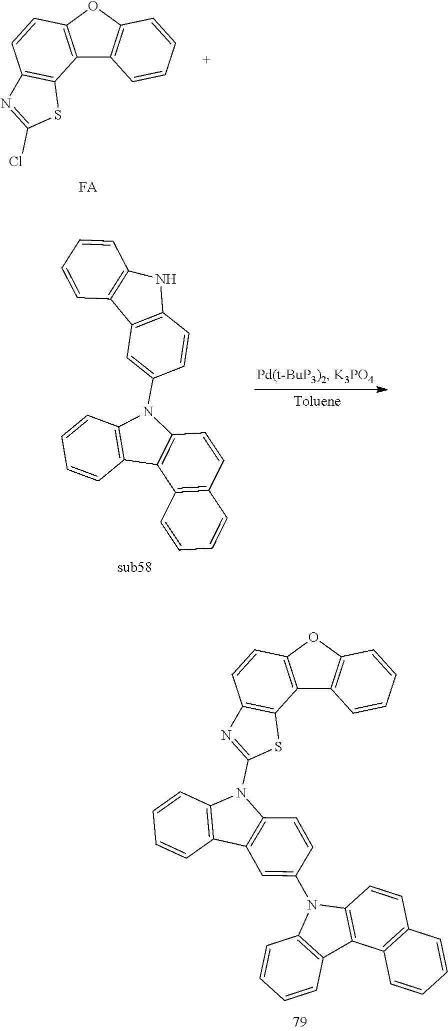 [SMILES: Clc1nc2ccc3oc4ccccc4c3c2s1 ; "FA" ; c1ccc2c(c1)ccc1c2c2ccccc2n1-c1ccc2[nH]c3ccccc3c2c1 ; c1ccc2c(c1)ccc1c2c2ccccc2n1-c1ccc2c(c1)c1ccccc1n2-c1nc2ccc3oc4ccccc4c3c2s1]

Compound FA (10 g, 38.5 mmol), sub58 (15.5 g, 40.4 mmol) and potassium phosphate (24.5 g, 115.5 mmol) were added to toluene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound 79 (14 g). (Yield: 60%, MS: [M+H]+=606)

### Example 80: Preparation of Compound 80

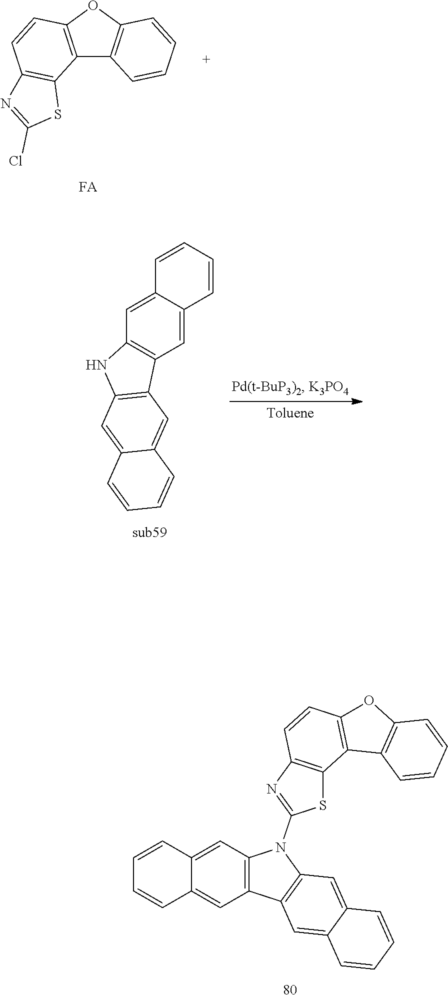 [SMILES: Clc1nc2ccc3oc4ccccc4c3c2s1 ; "FA" ; c1ccc2cc3c(cc2c1)[nH]c1cc2ccccc2cc13 ; c1ccc2cc3c(cc2c1)c1cc2ccccc2cc1n3-c1nc2ccc3oc4ccccc4c3c2s1]

Compound FA (10 g, 38.5 mmol), sub59 (10.8 g, 40.4 mmol) and potassium phosphate (24.5 g, 115.5 mmol) were added to toluene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound 80 (12.1 g). (Yield: 64%, MS: [M+H]+=491)

### Example 81: Preparation of Compound 81

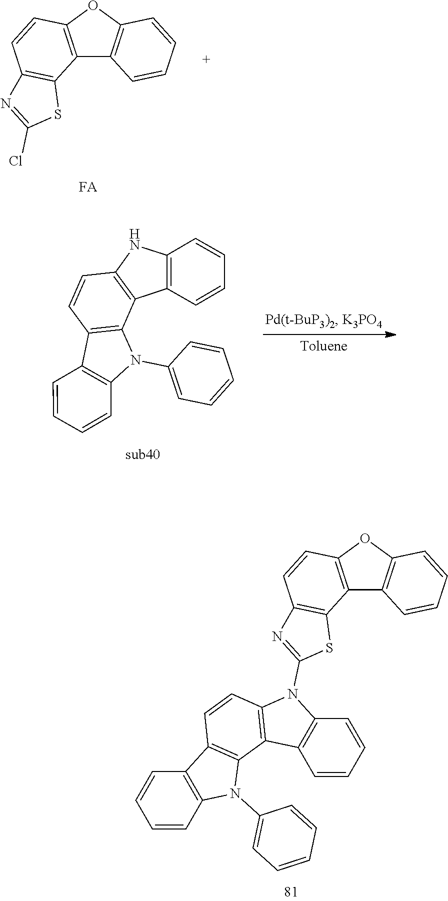 [SMILES: Clc1nc2ccc3oc4ccccc4c3c2s1 ; "FA" ; c1ccc(-n2c3ccccc3c3ccc4[nH]c5ccccc5c4c32)cc1 ; c1ccc(-n2c3ccccc3c3ccc4c(c5ccccc5n4-c4nc5ccc6oc7ccccc7c6c5s4)c32)cc1]

Compound FA (10 g, 38.5 mmol), sub40 (13.4 g, 40.4 mmol) and potassium phosphate (24.5 g, 115.5 mmol) were added to toluene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound 81 (15.4 g). (Yield: 72%, MS: [M+H]+=556)

### Example 82: Preparation of Compound 82

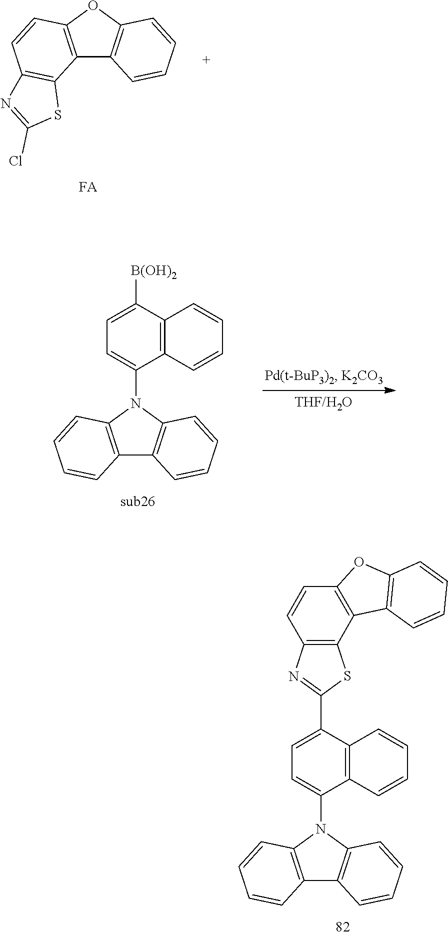 [SMILES: Clc1nc2ccc3oc4ccccc4c3c2s1 ; "FA" ; OB(O)c1ccc(-n2c3ccccc3c3ccccc32)c2ccccc12 ; c1ccc2c(c1)oc1ccc3nc(-c4ccc(-n5c6ccccc6c6ccccc65)c5ccccc45)sc3c12]

Compound FA (15 g, 57.8 mmol) and sub26 (20.4 g, 60.6 mmol) were added to THF (300 ml), and the mixture was stirred and refluxed. Then, potassium carbonate (23.9 g, 173.3 mmol) was dissolved in water (72 ml) and added thereto, and the mixture was sufficiently stirred, and then bis(tri-tert-butylphosphine)palladium(0) (0.3 g, 0.6 mmol) was added. After reacting for 2 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 82 (20.6 g). (Yield: 69%, MS: [M+H]+=517)

### Example 83: Preparation of Compound 83

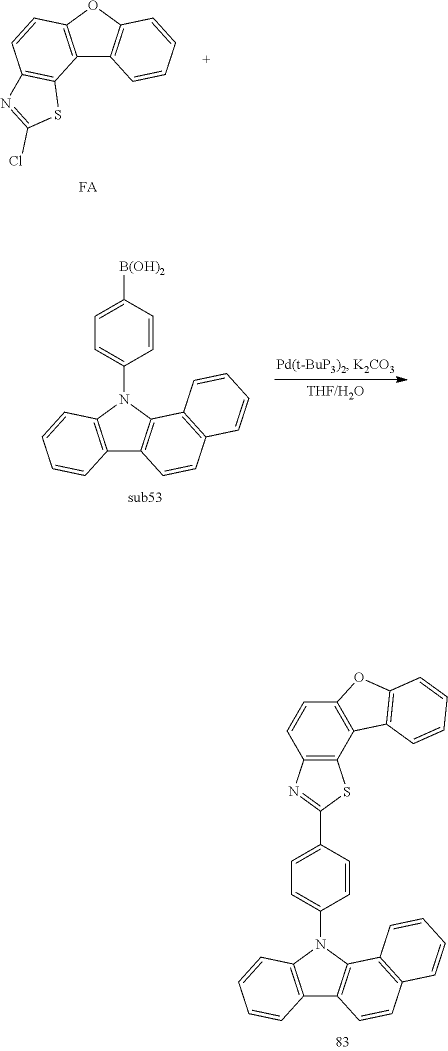 [SMILES: Clc1nc2ccc3oc4ccccc4c3c2s1 ; "FA" ; OB(O)c1ccc(-n2c3ccccc3c3ccc4ccccc4c32)cc1 ; c1ccc2c(c1)ccc1c3ccccc3n(-c3ccc(-c4nc5ccc6oc7ccccc7c6c5s4)cc3)c21]

Compound FA (15 g, 57.8 mmol) and sub53 (20.4 g, 60.6 mmol) were added to THF (300 ml), and the mixture was stirred and refluxed. Then, potassium carbonate (23.9 g, 173.3 mmol) was dissolved in water (72 ml) and added thereto, and the mixture was sufficiently stirred, and then bis(tri-tert-butylphosphine)palladium(0) (0.3 g, 0.6 mmol) was added. After reacting for 2 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 83 (18.8 g). (Yield: 63%, MS: [M+H]+=517)

### Example 84: Preparation of Compound 84

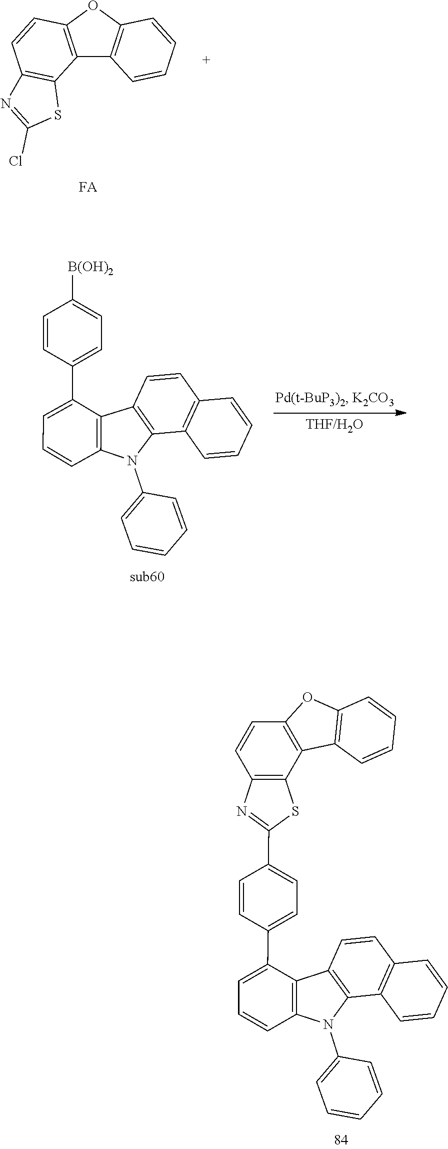 [SMILES: Clc1nc2ccc3oc4ccccc4c3c2s1 ; "FA" ; OB(O)c1ccc(-c2cccc3c2c2ccc4ccccc4c2n3-c2ccccc2)cc1 ; c1ccc(-n2c3cccc(-c4ccc(-c5nc6ccc7oc8ccccc8c7c6s5)cc4)c3c3ccc4ccccc4c32)cc1]

Compound FA (15 g, 57.8 mmol) and sub60 (25.1 g, 60.6 mmol) were added to THF (300 ml), and the mixture was stirred and refluxed. Then, potassium carbonate (23.9 g, 173.3 mmol) was dissolved in water (72 ml) and added thereto, and the mixture was sufficiently stirred, and then bis(tri-tert-butylphosphine)palladium(0) (0.3 g, 0.6 mmol) was added. After reacting for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 84 (22.9 g). (Yield: 67%, MS: [M+H]+=593)

### Example 85: Preparation of Compound 85

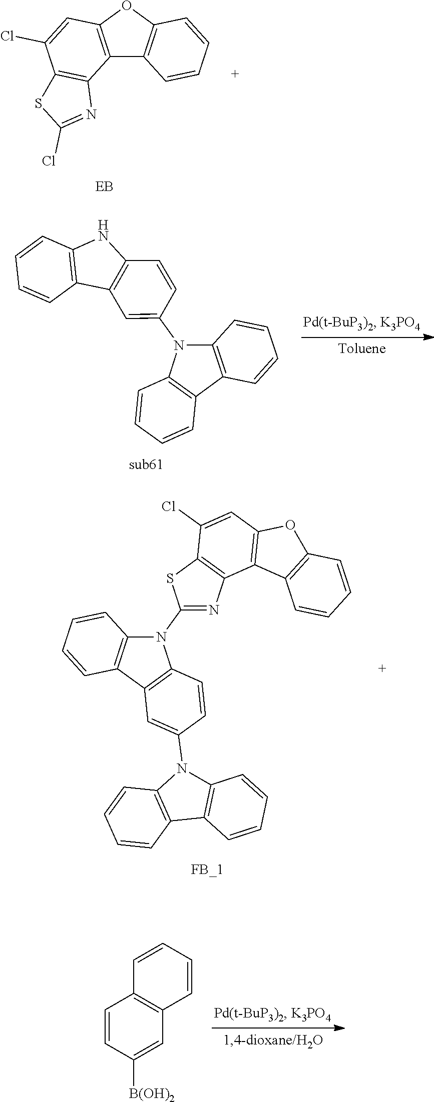

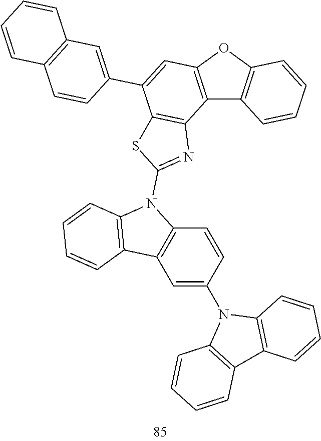 [SMILES: c1ccc2cc(-c3cc4oc5ccccc5c4c4nc(-n5c6ccccc6c6cc(-n7c8ccccc8c8ccccc87)ccc65)sc34)ccc2c1]

Compound EB (10 g, 34 mmol), sub61 (11.9 g, 35.7 mmol) and potassium phosphate (21.6 g, 102 mmol) were added to toluene (200 ml) under a nitrogen atmosphere, and the mixture was stirred and refluxed. Then, bis(tri-tert-butylphosphine)palladium(0) (0.2 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound FB_1 (14.8 g). (Yield: 74%, MS: [M+H]+=590)

Compound FB_1 (15 g, 25.4 mmol) and naphthalen-2-ylboronic acid (4.6 g, 26.7 mmol) were added to 1,4-dioxane (300 ml), and the mixture was stirred and refluxed. Then, potassium phosphate (16.2 g, 76.3 mmol) was dissolved in water (49 ml) and added thereto, and the mixture was sufficiently stirred, and then bis(tri-tert-butylphosphine)palladium(0) (0.1 g, 0.3 mmol) was added. After reacting for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 85 (11.6 g). (Yield: 67%, MS: [M+H]+=682)

### Example 86: Preparation of Compound 86

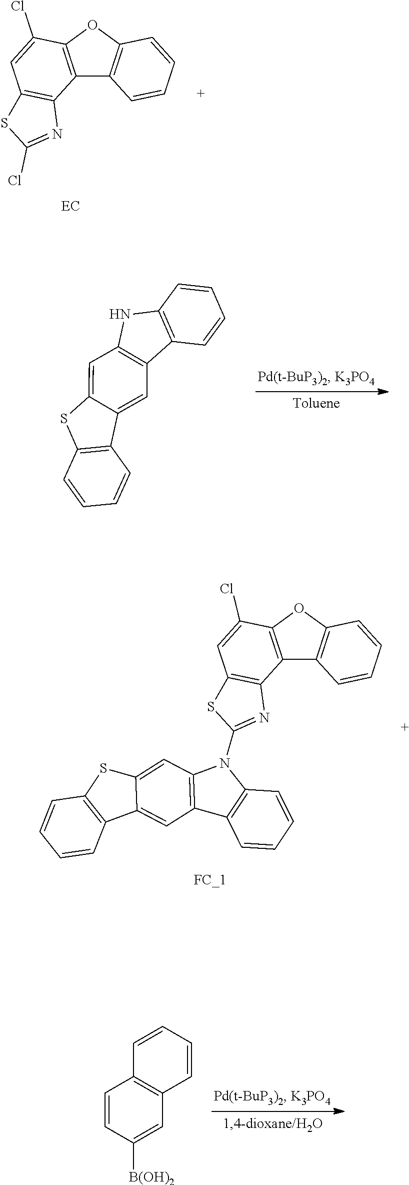 [SMILES: Clc1nc2c(cc(Cl)c3oc4ccccc4c32)s1 ; c1ccc2c(c1)[nH]c1cc3sc4ccccc4c3cc12 ; Clc1cc2sc(-n3c4ccccc4c4cc5c(cc43)sc3ccccc35)nc2c2c1oc1ccccc12 ; OB(O)c1ccc2ccccc2c1]

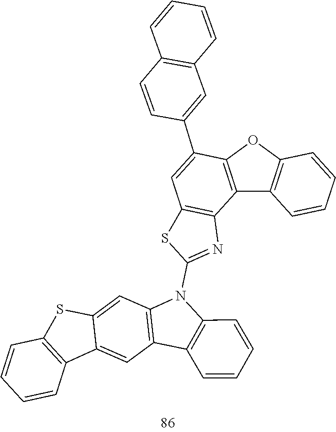 [SMILES: c1ccc2cc(-c3cc4sc(-n5c6ccccc6c6cc7c(cc65)sc5ccccc57)nc4c4c3oc3ccccc34)ccc2c1]

Compound EC (10 g, 34 mmol), sub62 (9.8 g, 35.7 mmol) and potassium phosphate (21.6 g, 102 mmol) were added to toluene (200 ml) under a nitrogen atmosphere, and the mixture was stirred and refluxed. Then, bis(tri-tert-butylphosphine)palladium(0) (0.2 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound FC_1 (10.8 g). (Yield: 60%, MS: [M+H]+=531)

Compound FC_1 (15 g, 28.3 mmol) and naphthalen-2-ylboronic acid (5.1 g, 29.7 mmol) were added to 1,4-dioxane (300 ml), and the mixture was stirred and refluxed. Then, potassium phosphate (18 g, 84.9 mmol) was dissolved in water (54 ml) and added thereto, and the mixture was sufficiently stirred, and then bis(tri-tert-butylphosphine)palladium(0) (0.1 g, 0.3 mmol) was added. After reacting for 2 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 86 (12.3 g). (Yield: 70%, MS: [M+H]+=623)

### Example 87: Preparation of Compound 87

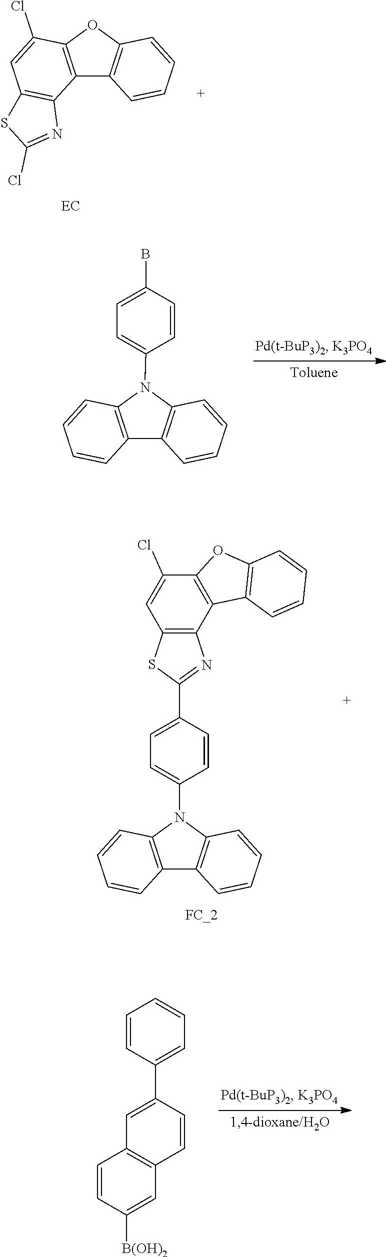

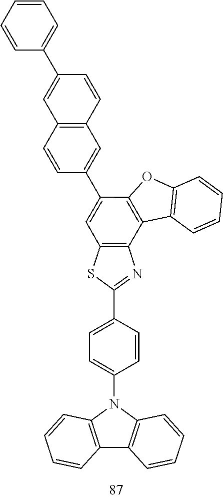 [SMILES: c1ccc(-c2ccc3cc(-c4cc5sc(-c6ccc(-n7c8ccccc8c8ccccc87)cc6)nc5c5c4oc4ccccc45)ccc3c2)cc1]

Compound EC (15 g, 51 mmol) and sub63 (15.4 g, 53.5 mmol) were added to THF (300 ml), and the mixture was stirred and refluxed. Then, potassium carbonate (21.1 g, 153 mmol) was dissolved in water (63 ml) and added thereto, and the mixture was sufficiently stirred, and then bis(tri-tert-butylphosphine)palladium(0) (0.3 g, 0.5 mmol) was added. After reacting for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound FC_2 (16.8 g). (Yield: 66%, MS: [M+H]+=501)

Compound FC_2 (15 g, 29.9 mmol) and (6-phenylnaphthalen-2-yl)boronic acid (7.8 g, 31.4 mmol) were added to 1,4-dioxane (300 ml), and the mixture was stirred and refluxed. Then, potassium phosphate (19.1 g, 89.8 mmol) was dissolved in water (57 ml) and added thereto, and the mixture was sufficiently stirred, and then bis(tri-tert-butylphosphine)palladium(0) (0.2 g, 0.3 mmol) was added. After reacting for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 87 (12.2 g). (Yield: 61%, MS: [M+H]+=669)

### Example 88: Preparation of Compound 88

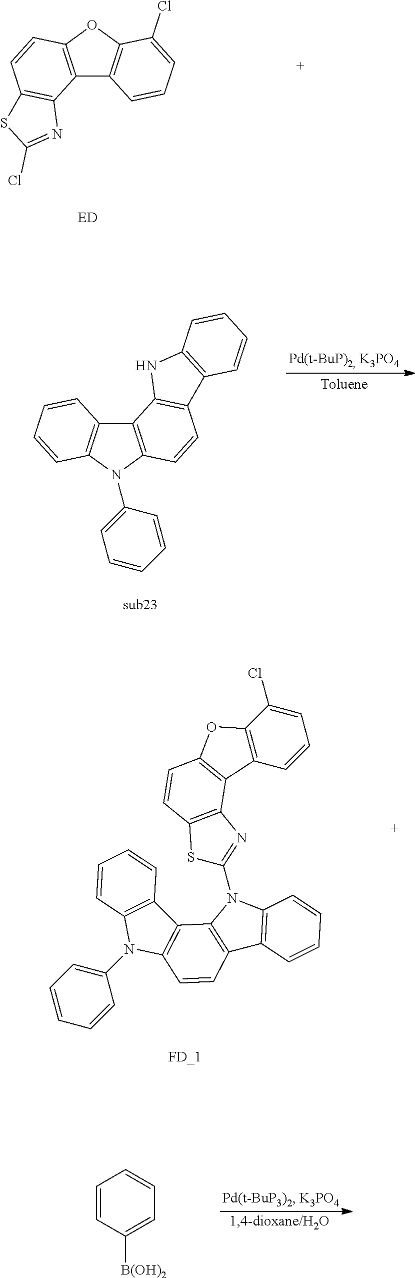 [SMILES: Clc1nc2c(ccc3oc4c(Cl)cccc4c32)s1 ; c1ccc(-n2c3ccccc3c3c4[nH]c5ccccc5c4ccc32)cc1 ; Clc1cccc2c1oc1ccc3sc(-n4c5ccccc5c5ccc6c(c7ccccc7n6-c6ccccc6)c54)nc3c12 ; OB(O)c1ccccc1]

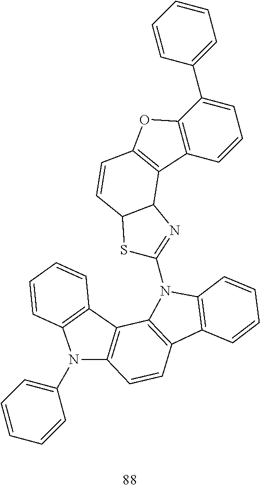 [SMILES: C1=CC2SC(n3c4ccccc4c4ccc5c(c6ccccc6n5-c5ccccc5)c43)=NC2c2c1oc1c(-c3ccccc3)cccc21]

Compound ED (10 g, 34 mmol), sub23 (11.9 g, 35.7 mmol) and potassium phosphate (21.6 g, 102 mmol) were added to toluene (200 ml) under a nitrogen atmosphere, and the mixture was stirred and refluxed. Then, bis(tri-tert-butylphosphine)palladium(0) (0.2 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound FD_1 (14.8 g). (Yield: 74%, MS: [M+H]+=590)

Compound FD_1 (15 g, 25.4 mmol) and phenylboronic acid (3.3 g, 26.7 mmol) were added to 1,4-dioxane (300 ml), and the mixture was stirred and refluxed. Then, potassium phosphate (16.2 g, 76.3 mmol) was dissolved in water (49 ml) and added thereto, and the mixture was sufficiently stirred, and then bis(tri-tert-butylphosphine)palladium(0) (0.1 g, 0.3 mmol) was added. After reacting for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 88 (9.8 g). (Yield: 61%, MS: [M+H]+=632)

### Example 89: Preparation of Compound 89

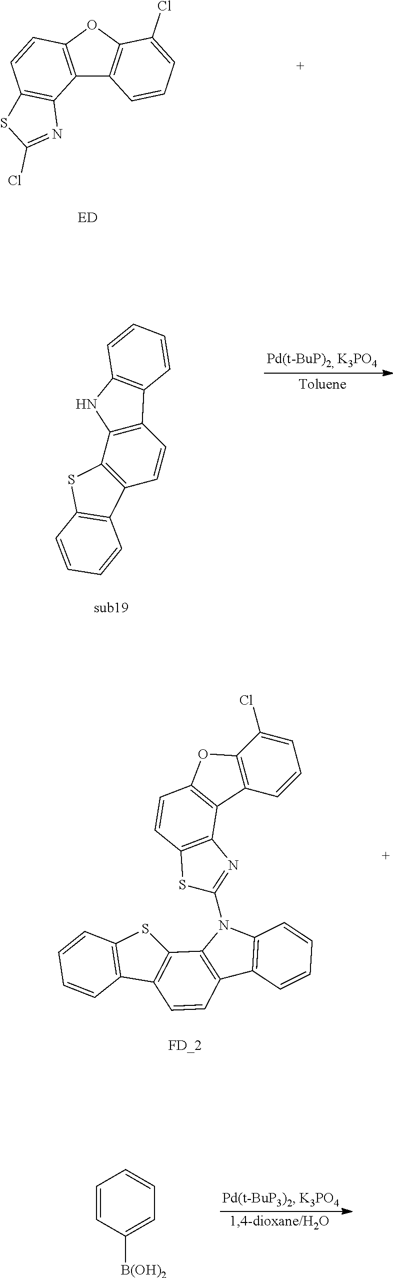

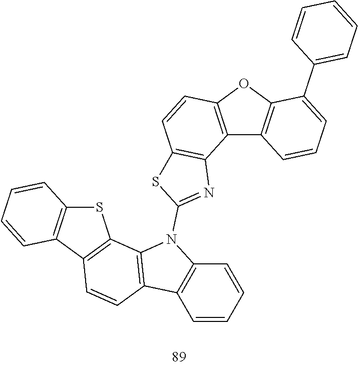 [SMILES: c1ccc(-c2cccc3c2oc2ccc4sc(-n5c6ccccc6c6ccc7c8ccccc8sc7c65)nc4c23)cc1]

Compound ED (10 g, 34 mmol), sub19 (9.8 g, 35.7 mmol) and potassium phosphate. (21.6 g, 102 mmol) were added to toluene (200 ml) under a nitrogen atmosphere, and the mixture was stirred and refluxed. Then, bis(tri-tert-butylphosphine)palladium(0) (0.2 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound FD_2 (13.3 g). (Yield: 74%, MS: [M+H]+=531)

Compound FD_2 (15 g, 28.2 mmol) and phenylboronic acid (3.6 g, 29.7 mmol) were added to 1,4-dioxane (300 ml), and the mixture was stirred and refluxed. Then, potassium phosphate (18 g, 84.7 mmol) was dissolved in water (54 ml) and added thereto, and the mixture was sufficiently stirred, and then bis(tri-tert-butylphosphine)palladium(0) (0.1 g, 0.3 mmol) was added. After reacting for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 89 (10.7 g). (Yield: 66%, MS: [M+H]+=573)

### Example 90: Preparation of Compound 90

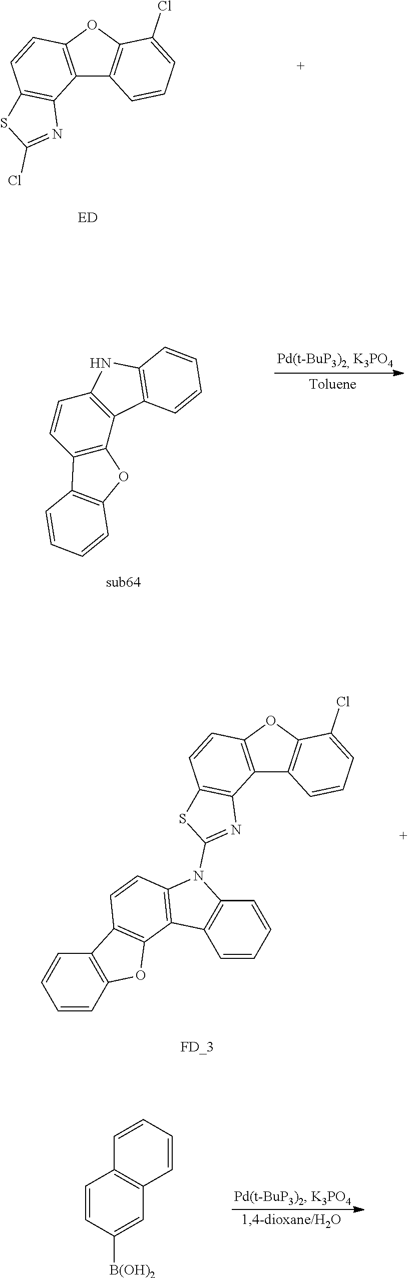 [SMILES: Clc1nc2c(ccc3oc4c(Cl)cccc4c32)s1 ; c1ccc2c(c1)[nH]c1ccc3c4ccccc4oc3c12 ; Clc1cccc2c1oc1ccc3sc(-n4c5ccccc5c5c6oc7ccccc7c6ccc54)nc3c12 ; OB(O)c1ccc2ccccc2c1]

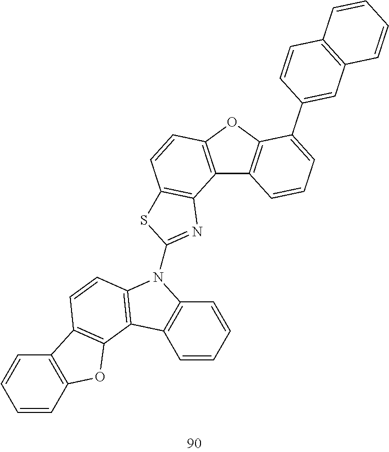 [SMILES: c1ccc2cc(-c3cccc4c3oc3ccc5sc(-n6c7ccccc7c7c8oc9ccccc9c8ccc76)nc5c34)ccc2c1]

Compound ED (10 g, 34 mmol), sub64 (9.2 g, 35.7 mmol) and potassium phosphate (21.6 g, 102 mmol) were added to toluene (200 ml) under a nitrogen atmosphere, and the mixture was stirred and refluxed. Then, bis(tri-tert-butylphosphine)palladium(0) (0.2 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound FD_3 (12.8 g). (Yield: 73%, MS: [M+H]+=515)

Compound FD_3 (15 g, 29.1 mmol) and naphthalen-2-ylboronic acid (5.3 g, 30.6 mmol) were added to 1,4-dioxane (300 ml), and the mixture was stirred and refluxed. Then, potassium phosphate (18.5 g, 87.4 mmol) was dissolved in water (56 ml) and added thereto, and the mixture was sufficiently stirred, and then bis(tri-tert-butylphosphine)palladium(0) (0.1 g, 0.3 mmol) was added. After reacting for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 90 (11.1 g). (Yield: 63%, MS: [M+H]+=607)

### Example 91: Preparation of Compound 91

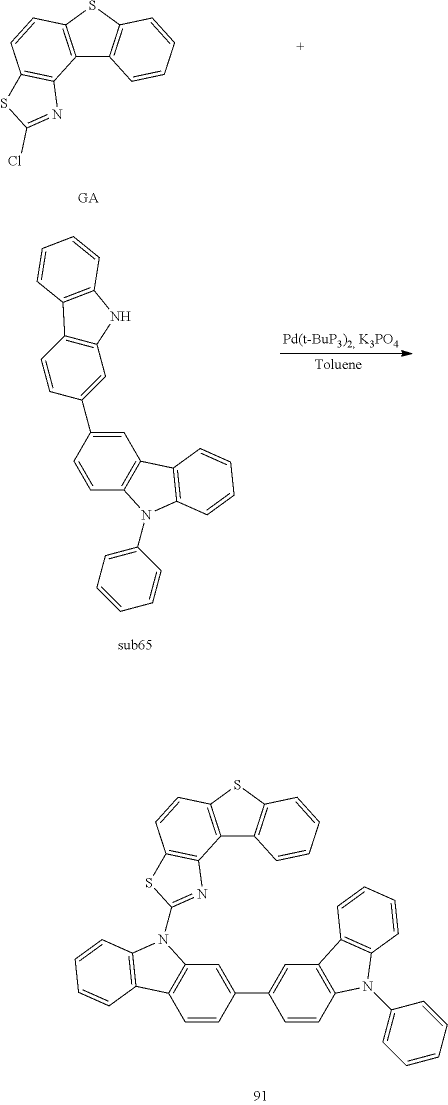 [SMILES: Clc1nc2c(ccc3sc4ccccc4c32)s1 ; c1ccc(-n2c3ccccc3c3cc(-c4ccc5c(c4)[nH]c4ccccc45)ccc32)cc1 ; c1ccc(-n2c3ccccc3c3cc(-c4ccc5c6ccccc6n(-c6nc7c(ccc8sc9ccccc9c87)s6)c5c4)ccc32)cc1]

Compound GA (10 g, 36.3 mmol), sub65 (15.6 g, 38.1 mmol) and potassium phosphate. (23.1 g, 108.8 mmol) were added to toluene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound 91 (14.1 g). (Yield: 60%, MS: [M+H]+=648)

### Example 92: Preparation of Compound 92

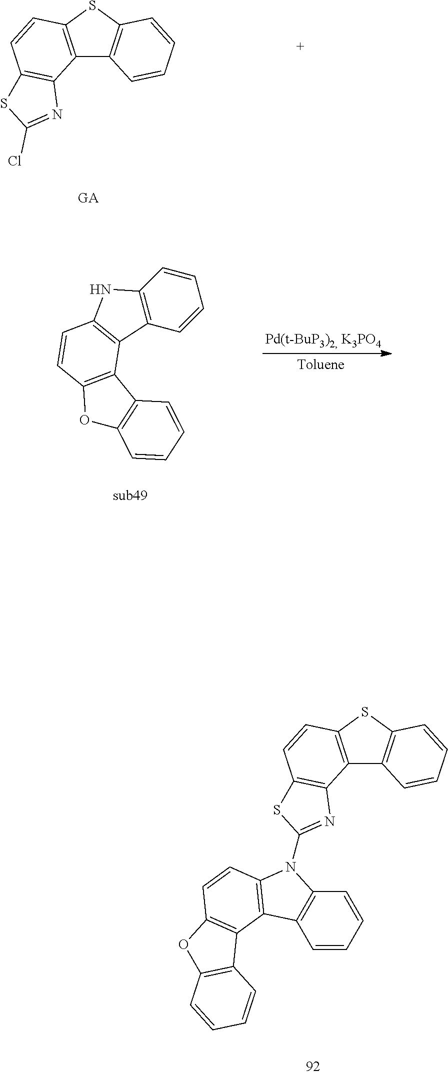 [SMILES: Clc1nc2c(ccc3sc4ccccc4c32)s1 ; c1ccc2c(c1)[nH]c1ccc3oc4ccccc4c3c12 ; c1ccc2c(c1)oc1ccc3c(c4ccccc4n3-c3nc4c(ccc5sc6ccccc6c54)s3)c12]

Compound GA (10 g, 36.3 mmol), sub49 (9.8 g, 38.1 mmol) and potassium phosphate (23.1 g, 108.8 mmol) were added to toluene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound 92 (11.9 g). (Yield: 66%, MS: [M+H]+=497)

### Example 93: Preparation of Compound 93

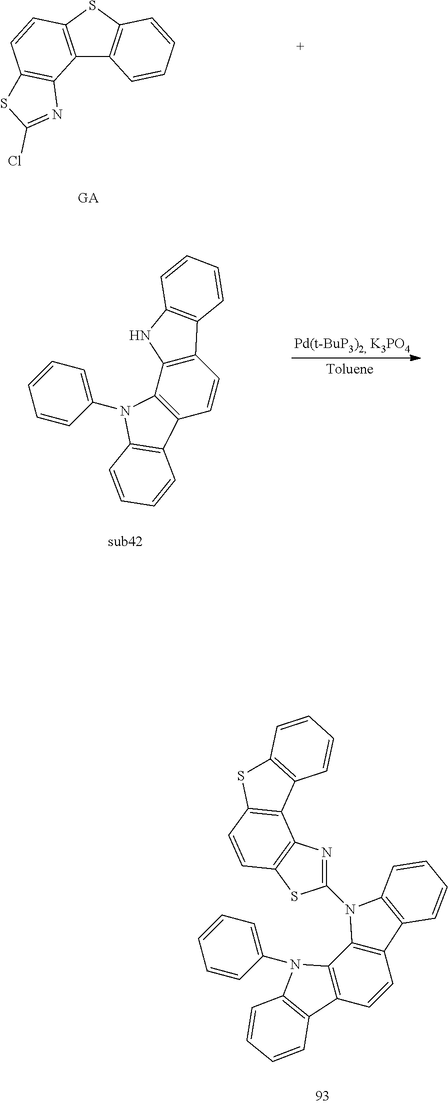 [SMILES: Clc1nc2c(ccc3sc4ccccc4c32)s1 ; c1ccc(-n2c3ccccc3c3ccc4c5ccccc5[nH]c4c32)cc1 ; c1ccc(-n2c3ccccc3c3ccc4c5ccccc5n(-c5nc6c(ccc7sc8ccccc8c76)s5)c4c32)cc1]

Compound GA (10 g, 36.3 mmol), sub42 (12.7 g, 38.1 mmol) and potassium phosphate (23.1 g, 108.8 mmol) were added to toluene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound 93 (14.3 g). (Yield: 69%, MS: [M+H]+=572)

### Example 94: Preparation of Compound 94

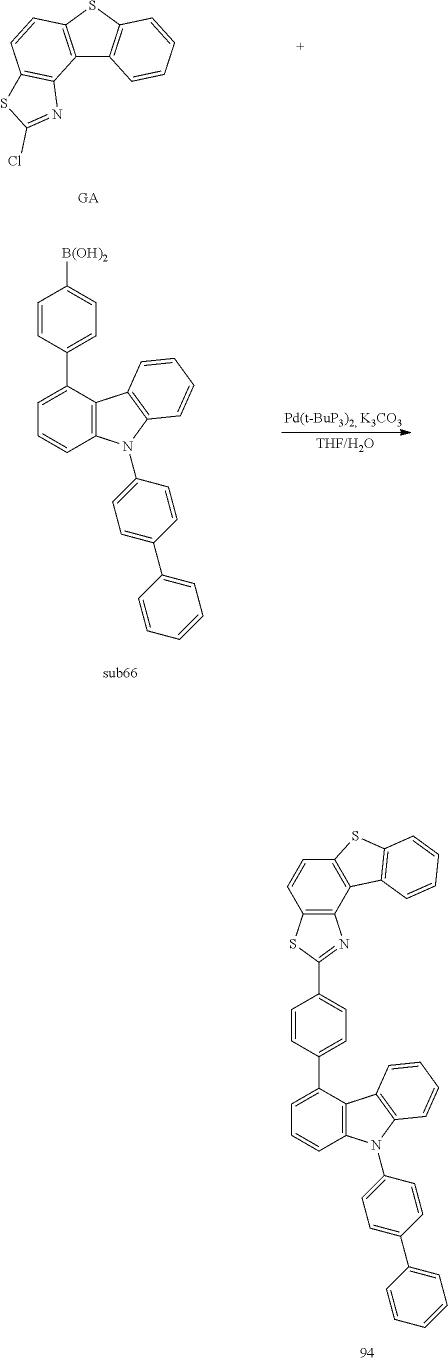 [SMILES: Clc1nc2c(ccc3sc4ccccc4c32)s1 ; OB(O)c1ccc(-c2cccc3c2c2ccccc2n3-c2ccc(-c3ccccc3)cc2)cc1 ; c1ccc(-c2ccc(-n3c4ccccc4c4c(-c5ccc(-c6nc7c(ccc8sc9ccccc9c87)s6)cc5)cccc43)cc2)cc1]

Compound GA (15 g, 54.4 mmol) and sub66 (25.1 g, 57.1 mmol) were added to THF (300 ml), and the mixture was stirred and refluxed. Then, potassium carbonate (22.6 g, 163.2 mmol) was dissolved in water (68 ml) and added thereto, and the mixture was sufficiently stirred, and then bis(tri-tert-butylphosphine)palladium(0) (0.3 g, 0.5 mmol) was added. After reacting for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 94 (25.9 g). (Yield: 75%, MS: [M+H]+=635)

### Example 95: Preparation of Compound 95

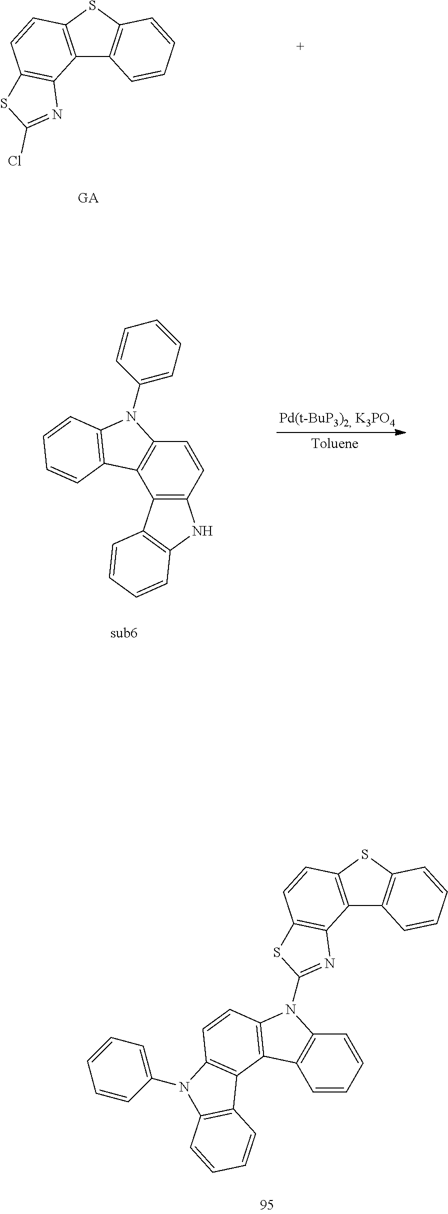 [SMILES: Clc1nc2c(ccc3sc4ccccc4c32)s1 ; c1ccc(-n2c3ccccc3c3c4c(ccc32)[nH]c2ccccc24)cc1 ; c1ccc(-n2c3ccccc3c3c4c5ccccc5n(-c5nc6c(ccc7sc8ccccc8c76)s5)c4ccc32)cc1]

Compound GA (10 g, 36.3 mmol), sub6 (12.7 g, 38.1 mmol) and potassium phosphate (23.1 g, 108.8 mmol) were added to toluene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound 95 (13.3 g). (Yield: 64%, MS: [M+H]+=572)

### Example 96: Preparation of Compound 96

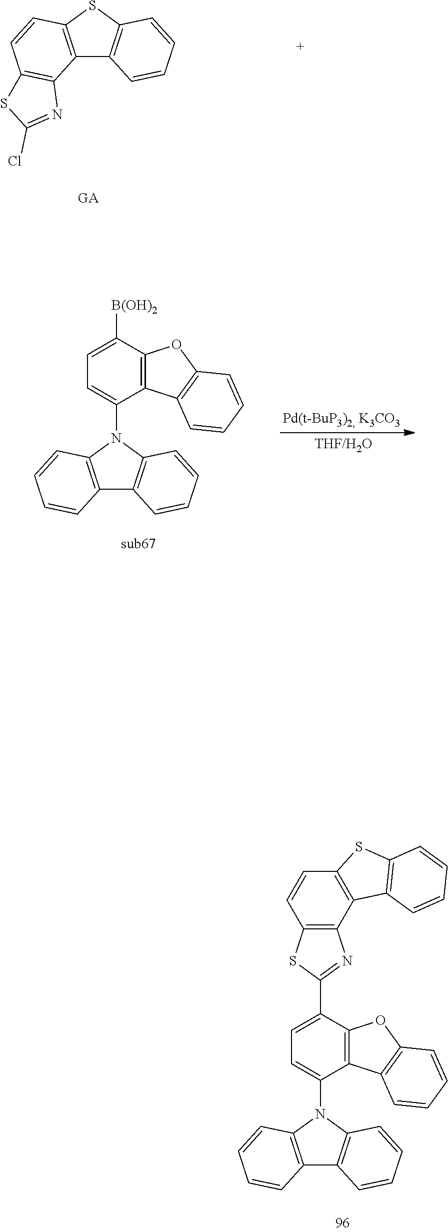 [SMILES: Clc1nc2c(ccc3sc4ccccc4c32)s1 ; OB(O)c1ccc(-n2c3ccccc3c3ccccc32)c2c1oc1ccccc12 ; c1ccc2c(c1)oc1c(-c3nc4c(ccc5sc6ccccc6c54)s3)ccc(-n3c4ccccc4c4ccccc43)c12]

Compound GA (15 g, 54.4 mmol) and sub67 (21.5 g, 57.1 mmol) were added to THF (300 ml), and the mixture was stirred and refluxed. Then, potassium carbonate (22.6 g, 163.2 mmol) was dissolved in water (68 ml) and added thereto, and the mixture was sufficiently stirred, and then bis(tri-tert-butylphosphine)palladium(0) (0.3 g, 0.5 mmol) was added. After reacting for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 96 (31.3 g). (Yield: 66%, MS: [M+H]+=873)

### Example 97: Preparation of Compound 97

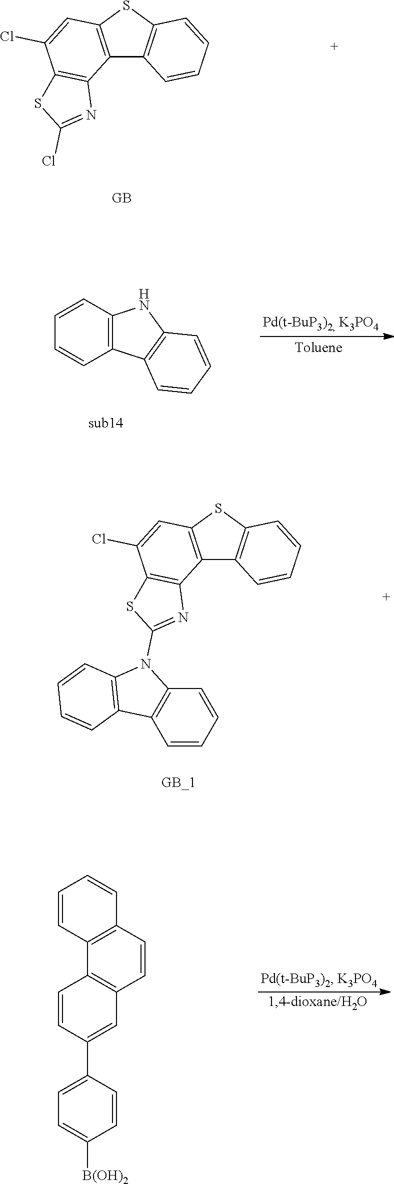 [SMILES: Clc1nc2c(s1)c(Cl)cc1sc3ccccc3c12 ; c1ccc2c(c1)[nH]c1ccccc12 ; Clc1cc2sc3ccccc3c2c2nc(-n3c4ccccc4c4ccccc43)sc12 ; OB(O)c1ccc(-c2ccc3c(ccc4ccccc43)c2)cc1]

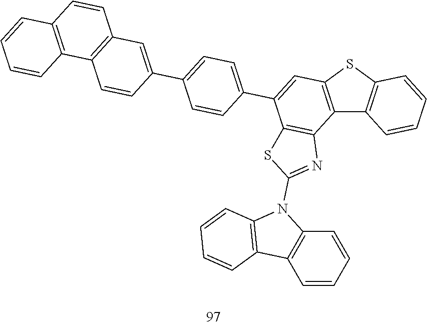 [SMILES: c1ccc2c(c1)ccc1cc(-c3ccc(-c4cc5sc6ccccc6c5c5nc(-n6c7ccccc7c7ccccc76)sc45)cc3)ccc12]

Compound GB (10 g, 32.2 mmol), sub14 (5.7 g, 33.8 mmol) and potassium phosphate (20.5 g, 96.7 mmol) were added to toluene (200 ml) under a nitrogen atmosphere, and the mixture was stirred and refluxed. Then, bis(tri-tert-butylphosphine)palladium(0) (0.2 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound GB_1 (9.9 g). (Yield: 70%, MS: [M+H]+=441)

Compound GB_1 (15 g, 34 mmol) and (4-(phenanthren-2-yl)phenyl)boronic acid (10.6 g, 35.7 mmol) were added to 1,4-dioxane (300 ml), and the mixture was stirred and refluxed. Then, potassium phosphate (21.7 g, 102.1 mmol) was dissolved in water (65 ml) and added thereto, and the mixture was sufficiently stirred, and then bis(tri-tert-butylphosphine)palladium(0) (0.2 g, 0.3 mmol) was added. After reacting for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 97 (16.6 g). (Yield: 74%, MS: [M+H]+=659)

### Example 98: Preparation of Compound 98

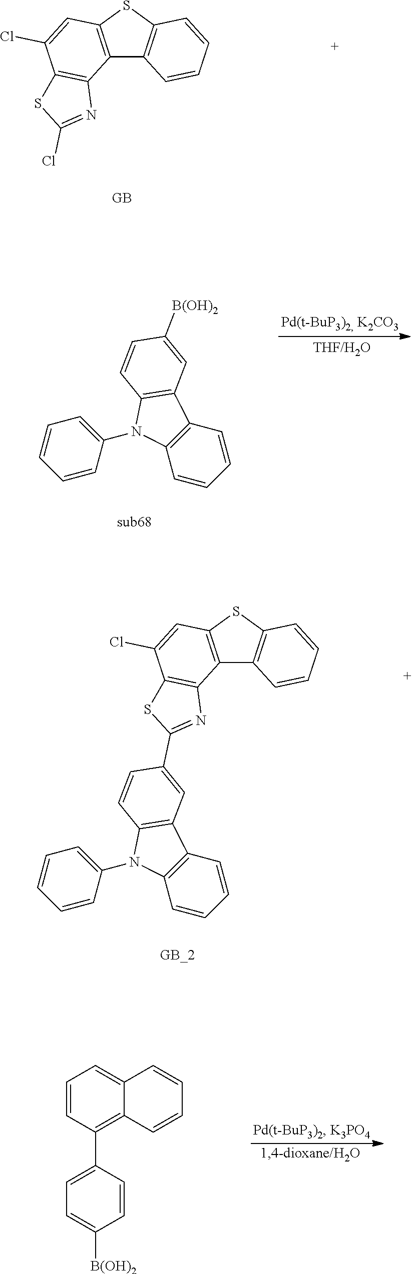 [SMILES: Clc1nc2c(s1)c(Cl)cc1sc3ccccc3c12 ; OB(O)c1ccc2c(c1)c1ccccc1n2-c1ccccc1 ; Clc1cc2sc3ccccc3c2c2nc(-c3ccc4c(c3)c3ccccc3n4-c3ccccc3)sc12 ; OB(O)c1ccc(-c2cccc3ccccc23)cc1]

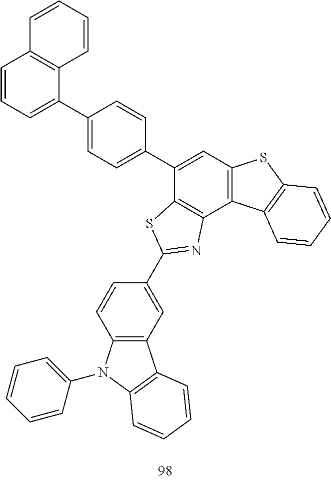 [SMILES: c1ccc(-n2c3ccccc3c3cc(-c4nc5c(s4)c(-c4ccc(-c6cccc7ccccc67)cc4)cc4sc6ccccc6c45)ccc32)cc1]

Compound GB (15 g, 48.4 mmol) and sub68 (14.6 g, 50.8 mmol) were added to THF (300 ml), and the mixture was stirred and refluxed. Then, potassium carbonate (20 g, 145.1 mmol) was dissolved in water (60 ml) and added thereto, and the mixture was sufficiently stirred, and then bis(tri-tert-butylphosphine)palladium(0) (0.2 g, 0.5 mmol) was added. After reacting for 2 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound GB_2 (15.7 g). (Yield: 63%, MS: [M+H]+=517)

Compound GB_2 (15 g, 29 mmol) and (4-(naphthalen-1-yl)phenyl)boronic acid (7.6 g, 30.5 mmol) were added to 1,4-dioxane (300 ml), and the mixture was stirred and refluxed. Then, potassium phosphate (18.5 g, 87 mmol) was dissolved in water (55 ml) and added thereto, and the mixture was sufficiently stirred, and then bis(tri-tert-butylphosphine)palladium(0) (0.1 g, 0.3 mmol) was added. After reacting for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 98 (11.9 g). (Yield: 60%, MS: [M+H]+=685)

### Example 99: Preparation of Compound 99

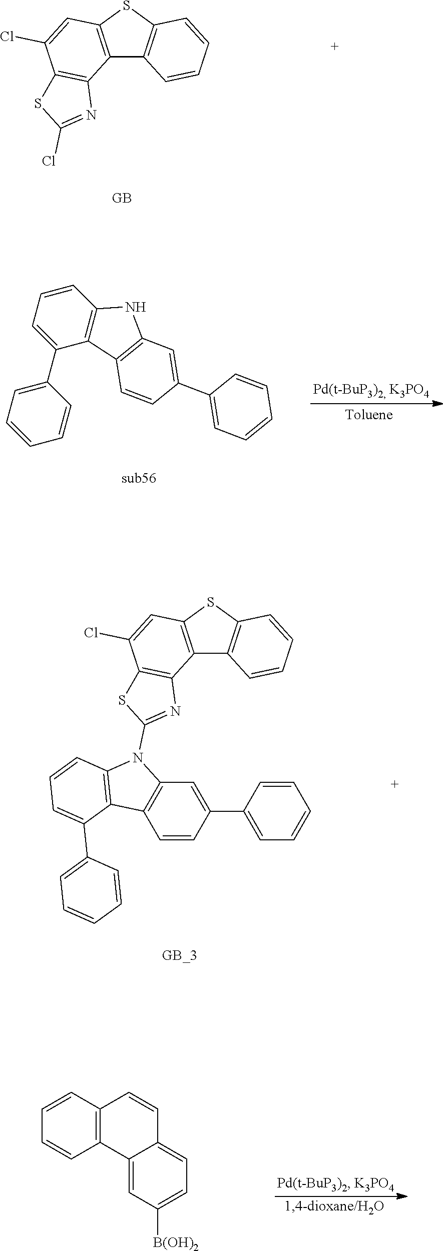 [SMILES: Clc1nc2c(s1)c(Cl)cc1sc3ccccc3c12 ; c1ccc(-c2ccc3c(c2)[nH]c2cccc(-c4ccccc4)c23)cc1 ; Clc1cc2sc3ccccc3c2c2nc(-n3c4cc(-c5ccccc5)ccc4c4c(-c5ccccc5)cccc43)sc12 ; OB(O)c1ccc2ccc3ccccc3c2c1]

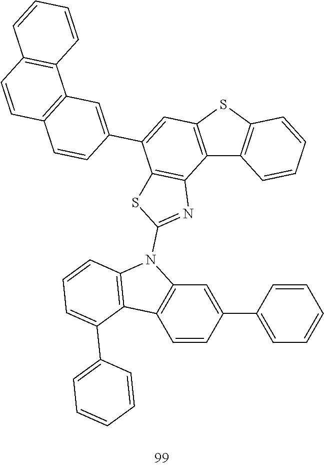 [SMILES: c1ccc(-c2ccc3c4c(-c5ccccc5)cccc4n(-c4nc5c(s4)c(-c4ccc6ccc7ccccc7c6c4)cc4sc6ccccc6c45)c3c2)cc1]

Compound GB (10 g, 32.2 mmol), sub56 (10.8 g, 33.8 mmol) and potassium phosphate (20.5 g, 96.7 mmol) were added to toluene (200 ml) under a nitrogen atmosphere, and the mixture was stirred and refluxed. Then, bis(tri-tert-butylphosphine)palladium(0) (0.2 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound GB_3 (13.7 g). (Yield: 72%, MS: [M+H]+=593)

Compound GB_3 (15 g, 25.3 mmol) and phenanthren-3-ylboronic acid (5.9 g, 26.6 mmol) were added to 1,4-dioxane (300 ml), and the mixture was stirred and refluxed. Then, potassium phosphate (16.1 g, 75.9 mmol) was dissolved in water (48 ml) and added thereto, and the mixture was sufficiently stirred, and then bis(tri-tert-butylphosphine)palladium(0) (0.1 g, 0.3 mmol) was added. After reacting for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 99 (11.9 g). (Yield: 64%, MS: [M+H]+=735)

### Example 100: Preparation of Compound 100

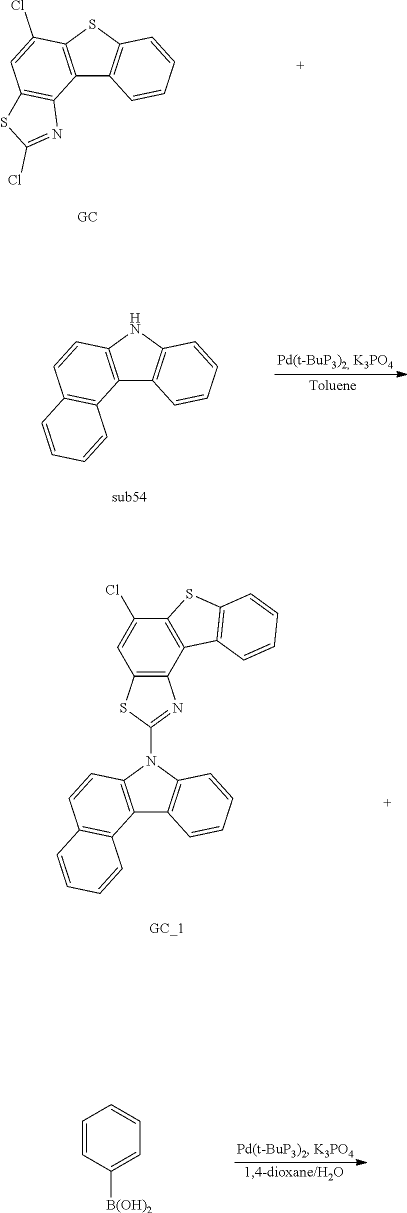 [SMILES: Clc1nc2c(cc(Cl)c3sc4ccccc4c32)s1 ; c1ccc2c(c1)ccc1[nH]c3ccccc3c12 ; Clc1cc2sc(-n3c4ccccc4c4c5ccccc5ccc43)nc2c2c1sc1ccccc12 ; OB(O)c1ccccc1]

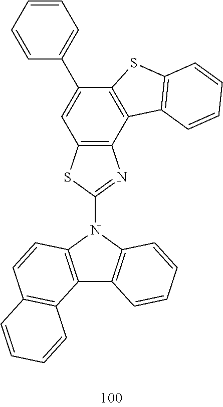 [SMILES: c1ccc(-c2cc3sc(-n4c5ccccc5c5c6ccccc6ccc54)nc3c3c2sc2ccccc23)cc1]

Compound GC (10 g, 32.2 mmol), sub54 (7.4 g, 33.8 mmol) and potassium phosphate (20.5 g, 96.7 mmol) were added to toluene (200 ml) under a nitrogen atmosphere, and the mixture was stirred and refluxed. Then, bis(tri-tert-butylphosphine)palladium(0) (0.2 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound GC_1 (10.1 g). (Yield: 64%, MS: [M+H]+=491)

Compound GC_1 (15 g, 30.5 mmol) and phenylboronic acid (3.9 g, 32.1 mmol) were added to 1,4-dioxane (300 ml), and the mixture was stirred and refluxed. Then, potassium phosphate (19.5 g, 91.6 mmol) was dissolved in water (58 ml) and added thereto, and the mixture was sufficiently stirred, and then bis(tri-tert-butylphosphine)palladium(0) (0.2 g, 0.3 mmol) was added. After reacting for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 100 (11.1 g). (Yield: 68%, MS: [M+H]+=533)

### Example 101: Preparation of Compound 101

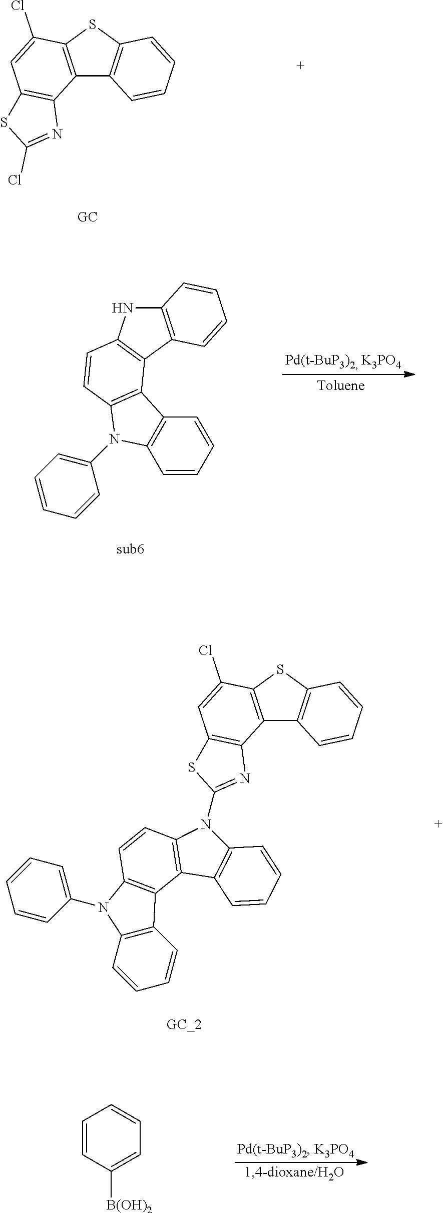 [SMILES: Clc1nc2c(cc(Cl)c3sc4ccccc4c32)s1 ; c1ccc(-n2c3ccccc3c3c4c(ccc32)[nH]c2ccccc24)cc1 ; Clc1cc2sc(-n3c4ccccc4c4c5c6ccccc6n(-c6ccccc6)c5ccc43)nc2c2c1sc1ccccc12 ; OB(O)c1ccccc1]

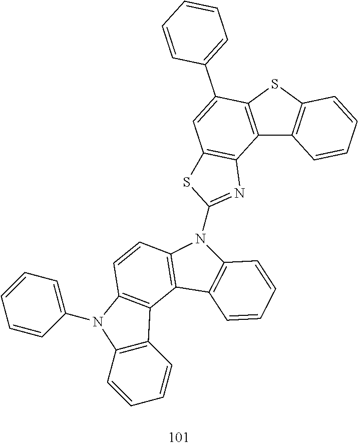 [SMILES: c1ccc(-c2cc3sc(-n4c5ccccc5c5c6c7ccccc7n(-c7ccccc7)c6ccc54)nc3c3c2sc2ccccc23)cc1]

Compound GC (10 g, 32.2 mmol), sub6 (11.3 g, 33.8 mmol) and potassium phosphate (20.5 g, 96.7 mmol) were added to toluene (200 ml) under a nitrogen atmosphere, and the mixture was stirred and refluxed. Then, bis(tri-tert-butylphosphine)palladium(0) (0.2 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound GC_2 (14 g). (Yield: 72%, MS: [M+H]+=606)

Compound GC_2 (15 g, 24.7 mmol) and phenylboronic acid (3.2 g, 26 mmol) were added to 1,4-dioxane (300 ml), and the mixture was stirred and refluxed. Then, potassium phosphate (15.8 g, 74.2 mmol) was dissolved in water (47 ml) and added thereto, and the mixture was sufficiently stirred, and then bis(tri-tert-butylphosphine)palladium(0) (0.1 g, 0.2 mmol) was added. After reacting for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 101 (12 g). (Yield: 75%, MS: [M+H]+=648)

### Example 102: Preparation of Compound 102

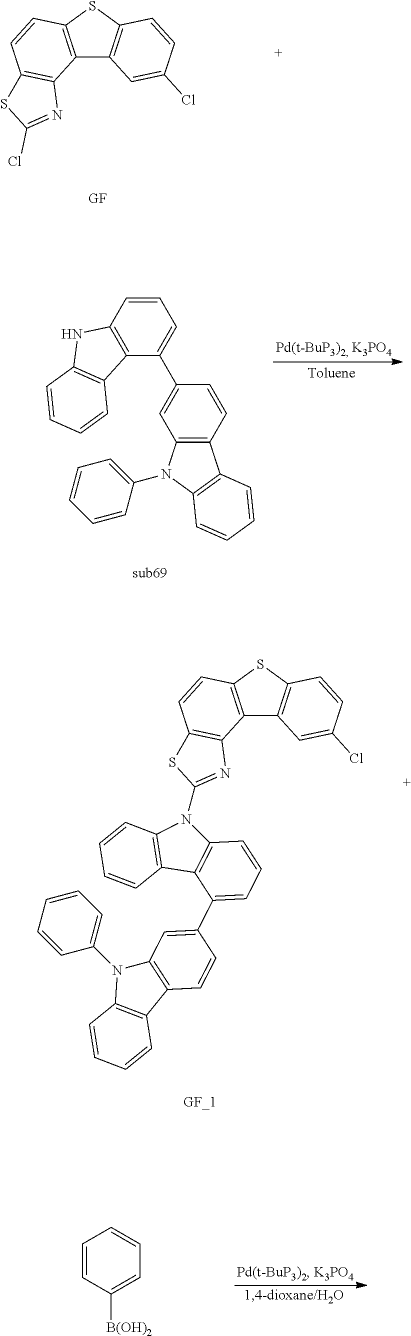 [SMILES: Clc1ccc2sc3ccc4sc(Cl)nc4c3c2c1 ; c1ccc(-n2c3ccccc3c3ccc(-c4cccc5[nH]c6ccccc6c45)cc32)cc1 ; Clc1ccc2sc3ccc4sc(-n5c6ccccc6c6c(-c7ccc8c9ccccc9n(-c9ccccc9)c8c7)cccc65)nc4c3c2c1 ; OB(O)c1ccccc1]

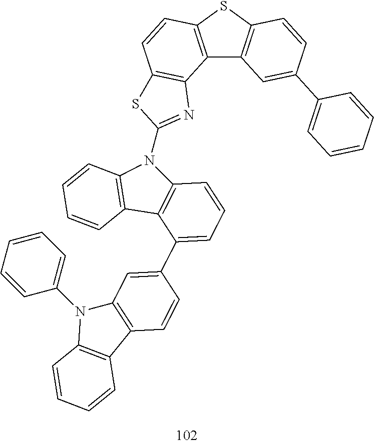 [SMILES: c1ccc(-c2ccc3sc4ccc5sc(-n6c7ccccc7c7c(-c8ccc9c%10ccccc%10n(-c%10ccccc%10)c9c8)cccc76)nc5c4c3c2)cc1]

Compound GF (10 g, 32.2 mmol), sub69 (13.8 g, 33.8 mmol) and potassium phosphate (20.5 g, 96.7 mmol) were added to toluene (200 ml) under a nitrogen atmosphere, and the mixture was stirred and refluxed. Then, bis(tri-tert-butylphosphine)palladium(0) (0.2 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound GF_1 (15.4 g). (Yield: 70%, MS: [M+H]+=682)

Compound GF_1 (15 g, 22 mmol) and phenylboronic acid (2.8 g, 23.1 mmol) were added to 1,4-dioxane (300 ml), and the mixture was stirred and refluxed. Then, potassium phosphate (14 g, 66 mmol) was dissolved in water (42 ml) and added thereto, and the mixture was sufficiently stirred, and then bis(tri-tert-butylphosphine)palladium(0) (0.1 g, 0.2 mmol) was added. After reacting for 2 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 102 (10.7 g). (Yield: 67%, MS: [M+H]+=724)

### Example 103: Preparation of Compound 103

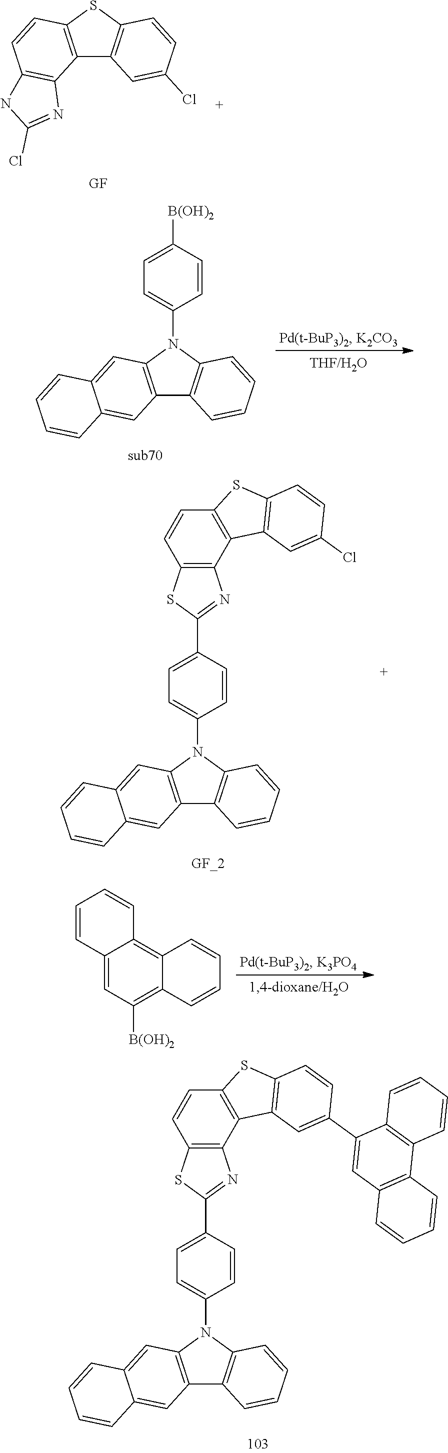

Compound GF (15 g, 48.4 mmol) and sub70 (17.1 g, 50.8 mmol) were added to THF (300 ml), and the mixture was stirred and refluxed. Then, potassium carbonate (20 g, 145.1 mmol) was dissolved in water (60 ml) and added thereto, and the mixture was sufficiently stirred, and then bis(tri-tert-butylphosphine)palladium(0) (0.2 g, 0.5 mmol) was added. After reacting for 2 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound GF_2 (17.5 g). (Yield: 64%, MS: [M+H]+=567)

Compound GF_2 (15 g, 26.4 mmol) and phenanthren-9-ylboronic acid (6.2 g, 27.8 mmol) were added to 1,4-dioxane (300 ml), and the mixture was stirred and refluxed. Then, potassium phosphate (16.8 g, 79.3 mmol) was dissolved in water (51 ml) and added thereto, and the mixture was sufficiently stirred, and then bis(tri-tert-butylphosphine)palladium(0) (0.1 g, 0.3 mmol) was added. After reacting for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 103 (12.7 g). (Yield: 68%, MS: [M+H]+=709)

### Example 104: Preparation of Compound 104

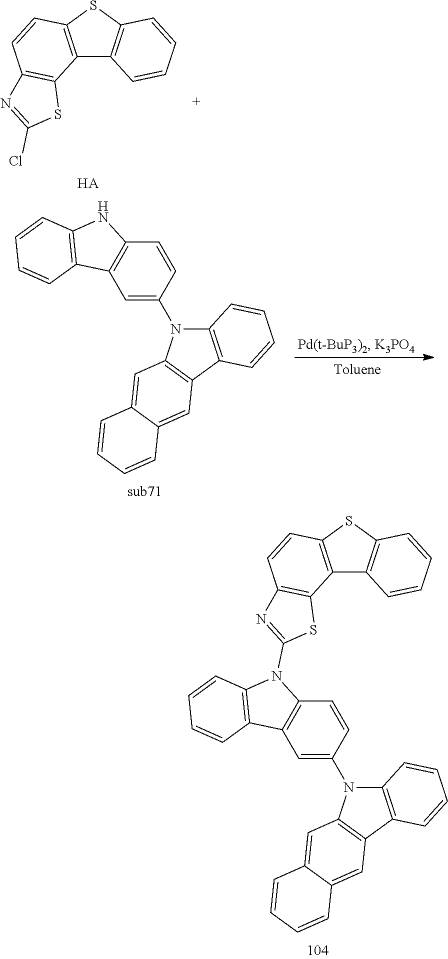

Compound HA (10 g, 36.3 mmol), sub71 (12.5 g, 38.1 mmol) and potassium phosphate (23.1 g, 108.8 mmol) were added to toluene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound 104 (14 g). (Yield: 62%, MS: [M+H]+=622)

### Example 105: Preparation of Compound 105

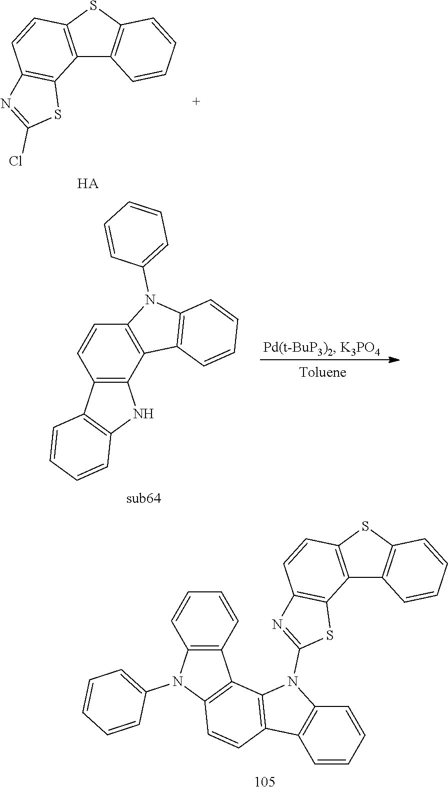

Compound HA (10 g, 36.3 mmol), sub64 (12.7 g, 38.1 mmol) and potassium phosphate (23.1 g, 108.8 mmol) were added to toluene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound 105 (12.6 g). (Yield: 61%, MS: [M+H]+=572)

### Example 106: Preparation of Compound 106

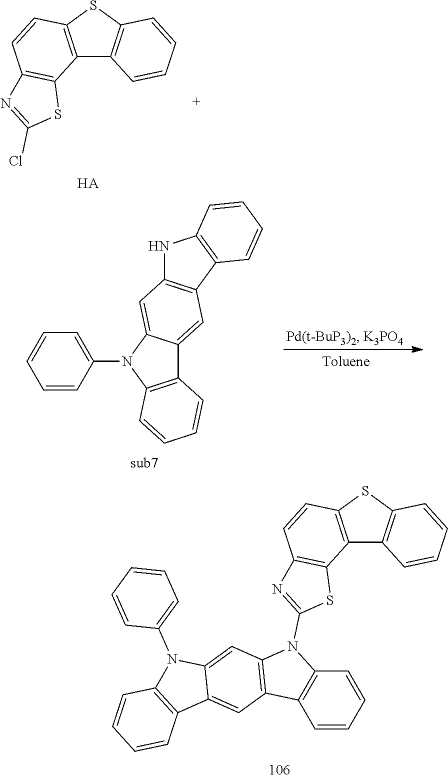 [SMILES: Clc1nc2ccc3sc4ccccc4c3c2s1 ; "HA" ; c1ccc(-n2c3ccccc3c3cc4c(cc32)[nH]c2ccccc24)cc1 ; c1ccc(-n2c3ccccc3c3cc4c5ccccc5n(-c5nc6ccc7sc8ccccc8c7c6s5)c4cc32)cc1]

Compound HA (10 g, 36.3 mmol), sub7 (12.7 g, 38.1 mmol) and potassium phosphate (23.1 g, 108.8 mmol) were added to toluene (200 ml) under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound 106 (13.9 g). (Yield: 67%, MS: [M+H]+=572)

### Example 107: Preparation of Compound 107

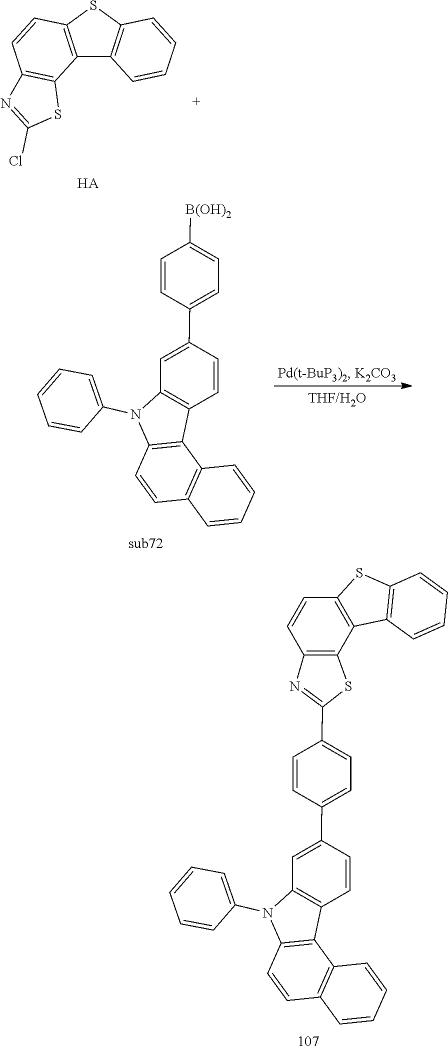 [SMILES: Clc1nc2ccc3sc4ccccc4c3c2s1 ; "HA" ; OB(O)c1ccc(-c2ccc3c4c5ccccc5ccc4n(-c4ccccc4)c3c2)cc1 ; c1ccc(-n2c3cc(-c4ccc(-c5nc6ccc7sc8ccccc8c7c6s5)cc4)ccc3c3c4ccccc4ccc32)cc1]

Compound HA (15 g, 54.4 mmol) and sub72 (23.6 g, 57.1 mmol) were added to THF (300 ml), and the mixture was stirred and refluxed. Then, potassium carbonate (22.6 g, 163.2 mmol) was dissolved in water (68 ml) and added thereto, and the mixture was sufficiently stirred, and then bis(tri-tert-butylphosphine)palladium(0) (0.3 g, 0.5 mmol) was added. After reacting for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 107 (24.8 g). (Yield: 75%, MS: [M+H]+=609)

### Example 108: Preparation of Compound 108

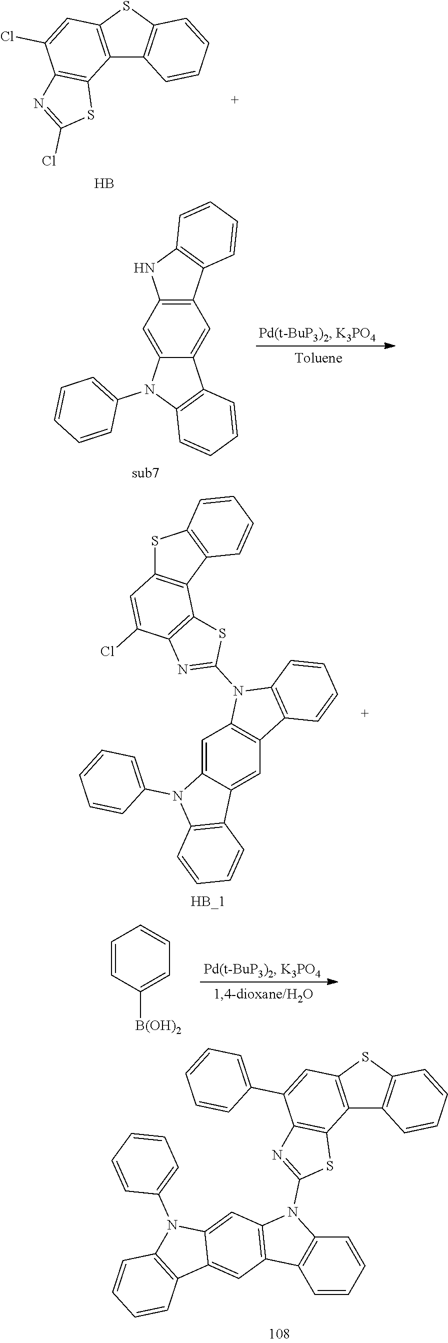

Compound HB (10 g, 32.2 mmol), sub7 (11.3 g, 33.8 mmol) and potassium phosphate (20.5 g, 96.7 mmol) were added to toluene (200 ml) under a nitrogen atmosphere, and the mixture was stirred and refluxed. Then, bis(tri-tert-butylphosphine)palladium(0) (0.2 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound HB_1 (12.1 g). (Yield: 62%, MS: [M+H]+=606)

Compound HB_1 (15 g, 24.7 mmol) and phenylboronic acid (3.2 g, 26 mmol) were added to 1,4-dioxane (300 ml), and the mixture was stirred and refluxed. Then, potassium phosphate (15.8 g, 74.2 mmol) was dissolved in water (47 ml) and added thereto, and the mixture was sufficiently stirred, and then bis(tri-tert-butylphosphine)palladium(0) (0.1 g, 0.2 mmol) was added. After reacting for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 108 (10.7 g). (Yield: 67%, MS: [M+H]+=648)

### Example 109: Preparation of Compound 109

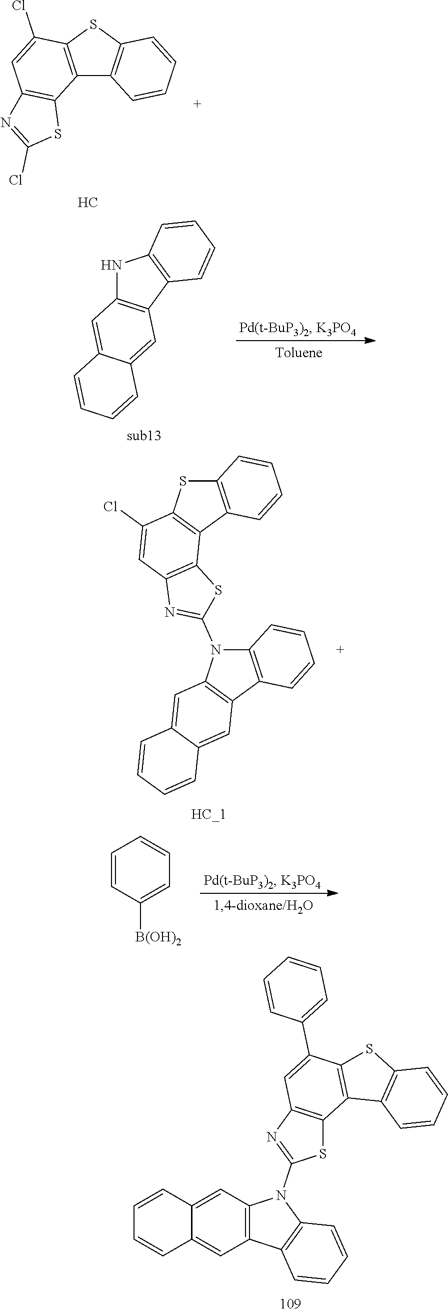

Compound HC (10 g, 32.2 mmol), sub13 (7.4 g, 33.8 mmol) and potassium phosphate. (20.5 g, 96.7 mmol) were added to toluene (200 ml) under a nitrogen atmosphere, and the mixture was stirred and refluxed. Then, bis(tri-tert-butylphosphine)palladium(0) (0.2 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound HC_1 (10.4 g). (Yield: 66%, MS: [M+H]+=491)

Compound HC_1 (15 g, 30.5 mmol) and phenylboronic acid (3.9 g, 32.1 mmol) were added to 1,4-dioxane (300 ml), and the mixture was stirred and refluxed. Then, potassium phosphate (19.5 g, 91.6 mmol) was dissolved in water (58 ml) and added thereto, and the mixture was sufficiently stirred, and then bis(tri-tert-butylphosphine)palladium(0) (0.2 g, 0.3 mmol) was added. After reacting for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 109 (11.2 g). (Yield: 69%, MS: [M+H]+=533)

### Example 110: Preparation of Compound 110

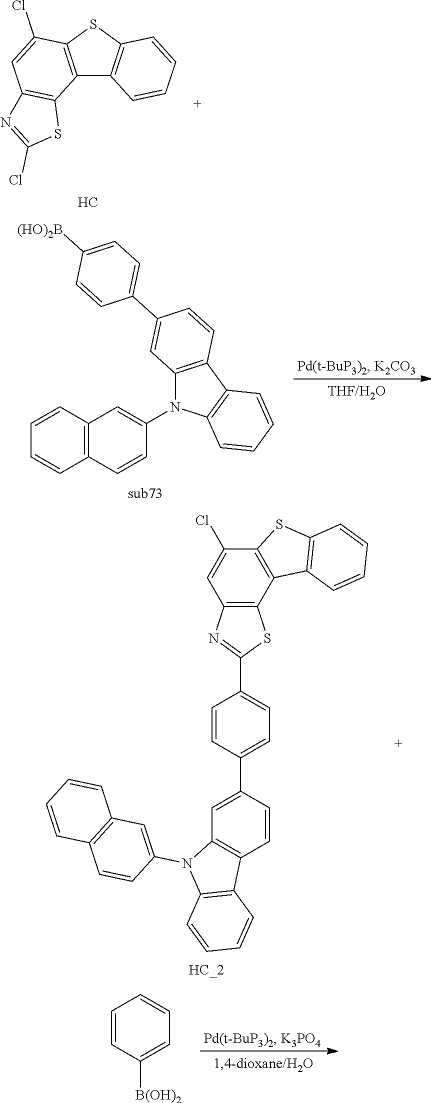

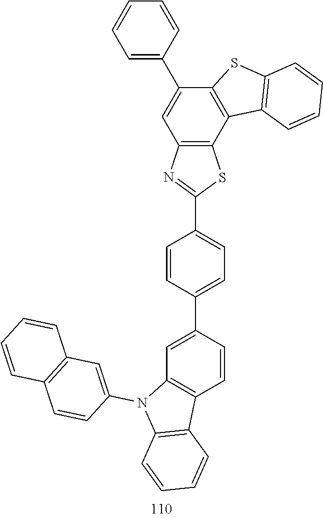 [SMILES: c1ccc(-c2cc3nc(-c4ccc(-c5ccc6c7ccccc7n(-c7ccc8ccccc8c7)c6c5)cc4)sc3c3c2sc2ccccc23)cc1]

Compound HC (15 g, 48.4 mmol) and sub73 (21 g, 50.8 mmol) were added to THF (300 ml), and the mixture was stirred and refluxed. Then, potassium carbonate (20 g, 145.1 mmol) was dissolved in water (60 ml) and added thereto, and the mixture was sufficiently stirred, and then bis(tri-tert-butylphosphine)palladium(0) (0.2 g, 0.5 mmol) was added. After reacting for 2 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound HC_2 (19.9 g). (Yield: 64%, MS: [M+H]+=643)

Compound HC_2 (15 g, 23.3 mmol) and phenylboronic acid (3 g, 24.5 mmol) were added to 1,4-dioxane (300 ml), and the mixture was stirred and refluxed. Then, potassium phosphate (14.9 g, 70 mmol) was dissolved in water (45 ml) and added thereto, and the mixture was sufficiently stirred, and then bis(tri-tert-butylphosphine)palladium(0) (0.1 g, 0.2 mmol) was added. After reacting for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 110 (12 g). (Yield: 75%, MS: [M+H]+=685)

### Example 111: Preparation of Compound 111

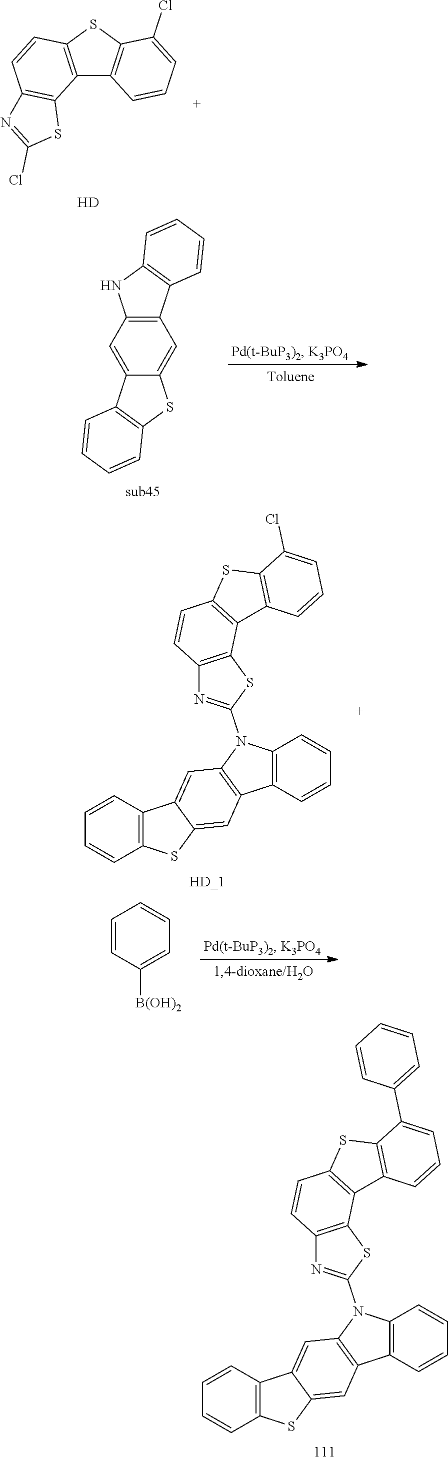

Compound HD (10 g, 32.2 mmol), sub45 (9.3 g, 33.8 mmol) and potassium phosphate. (20.5 g, 96.7 mmol) were added to toluene (200 ml) under a nitrogen atmosphere, and the mixture was stirred and refluxed. Then, bis(tri-tert-butylphosphine)palladium(0) (0.2 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound HD_1 (13 g). (Yield: 74%, MS: [M+H]+=547)

Compound HD_1 (15 g, 27.4 mmol) and phenylboronic acid (3.5 g, 28.8 mmol) were added to 1,4-dioxane (300 ml), and the mixture was stirred and refluxed. Then, potassium phosphate (17.5 g, 82.3 mmol) was dissolved in water (52 ml) and added thereto, and the mixture was sufficiently stirred, and then bis(tri-tert-butylphosphine)palladium(0) (0.1 g, 0.3 mmol) was added. After reacting for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 111 (10 g). (Yield: 62%, MS: [M+H]+=589)

### Example 112: Preparation of Compound 112

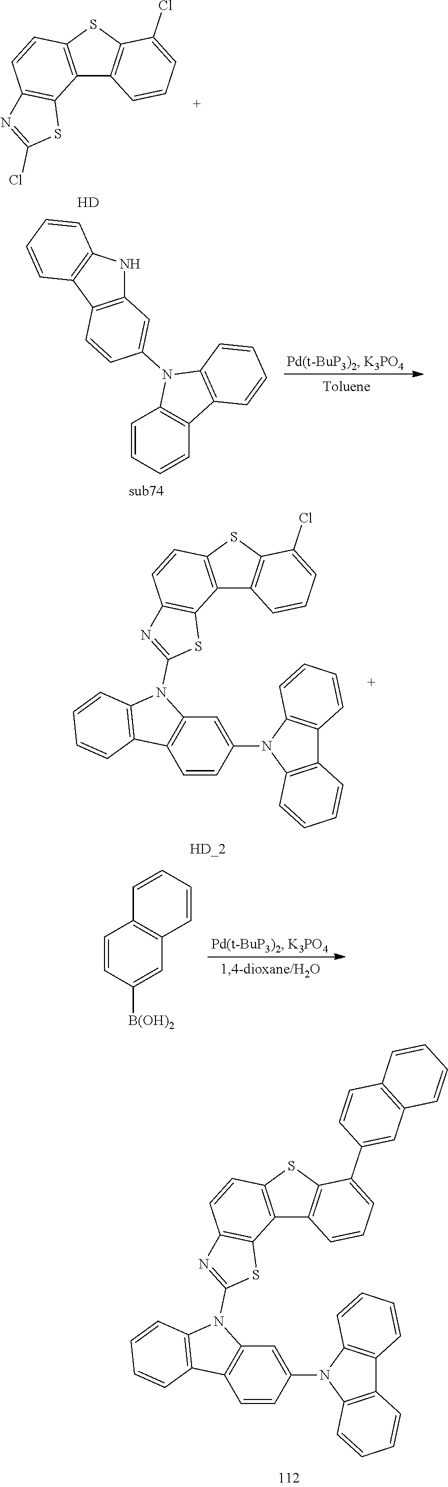

Compound HD (10 g, 32.2 mmol), sub74 (11.3 g, 33.8 mmol) and potassium phosphate (20.5 g, 96.7 mmol) were added to toluene (200 ml) under a nitrogen atmosphere, and the mixture was stirred and refluxed. Then, bis(tri-tert-butylphosphine)palladium(0) (0.2 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound HD_2 (11.7 g). (Yield: 60%, MS: [M+H]+=606)

Compound HD_2 (15 g, 24.7 mmol) and naphthalen-2-ylboronic acid (4.5 g, 26 mmol) were added to 1,4-dioxane (300 ml), and the mixture was stirred and refluxed. Then, potassium phosphate (15.8 g, 74.2 mmol) was dissolved in water (47 ml) and added thereto, and the mixture was sufficiently stirred, and then bis(tri-tert-butylphosphine)palladium(0) (0.1 g, 0.2 mmol) was added. After reacting for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 112 (12.8 g). (Yield: 74%, MS: [M+H]+=698)

### Example 113: Preparation of Compound 113

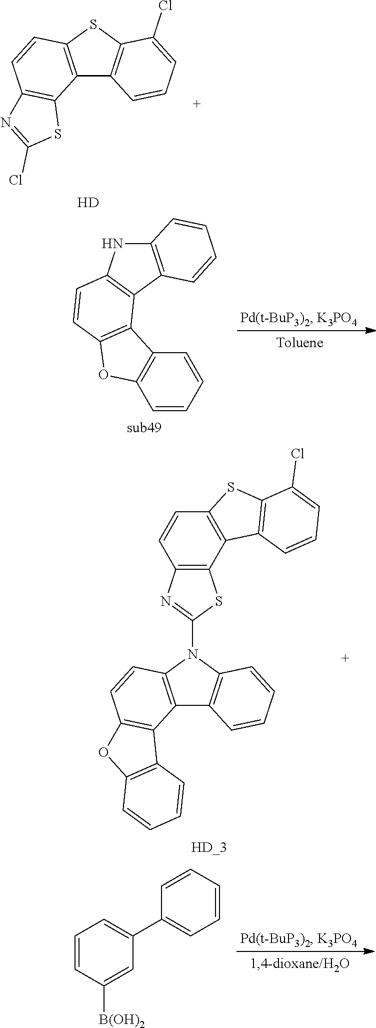

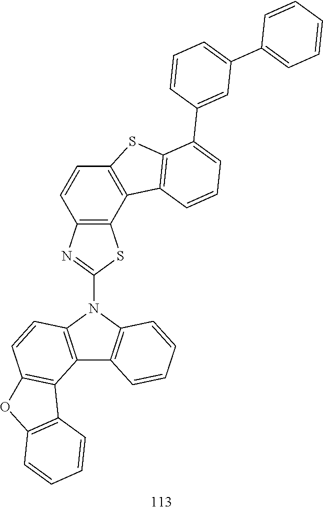 [SMILES: c1ccc(-c2cccc(-c3cccc4c3sc3ccc5nc(-n6c7ccccc7c7c8c(ccc76)oc6ccccc68)sc5c34)c2)cc1]

Compound HD (10 g, 32.2 mmol), sub49 (8.7 g, 33.8 mmol) and potassium phosphate (20.5 g, 96.7 mmol) were added to toluene (200 ml) under a nitrogen atmosphere, and the mixture was stirred and refluxed. Then, bis(tri-tert-butylphosphine)palladium(0) (0.2 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound HD_3 (12.3 g). (Yield: 72%, MS: [M+H]+=531)

Compound HD_3 (15 g, 28.2 mmol) and [1,1′-biphenyl]-3-ylboronic acid (5.9 g, 29.7 mmol) were added to 1,4-dioxane (300 ml), and the mixture was stirred and refluxed. Then, potassium phosphate (18 g, 84.7 mmol) was dissolved in water (54 ml) and added thereto, and the mixture was sufficiently stirred, and then bis(tri-tert-butylphosphine)palladium(0) (0.1 g, 0.3 mmol) was added. After reacting for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 113 (13.7 g). (Yield: 75%, MS: [M+H]+=649)

### Example 114: Preparation of Compound 114

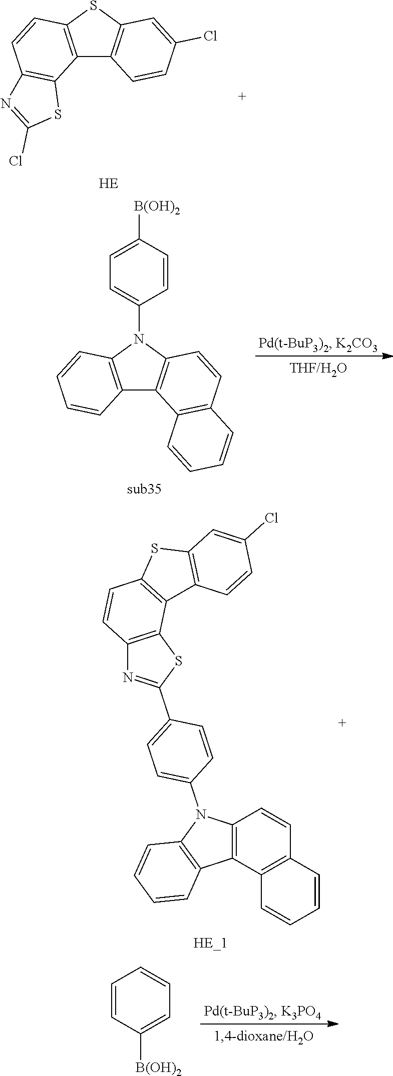

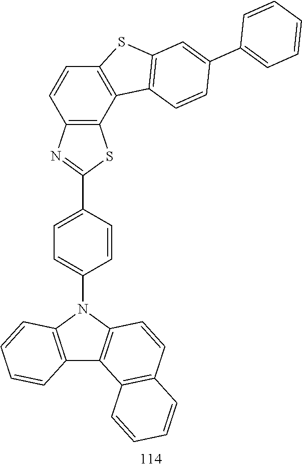 [SMILES: c1ccc(-c2ccc3c(c2)sc2ccc4nc(-c5ccc(-n6c7ccccc7c7c8ccccc8ccc76)cc5)sc4c23)cc1]

Compound HE (15 g, 48.4 mmol) and sub35 (17.1 g, 50.8 mmol) were added to THF (300 ml), and the mixture was stirred and refluxed. Then, potassium carbonate (20 g, 145.1 mmol) was dissolved in water (60 ml) and added thereto, and the mixture was sufficiently stirred, and then bis(tri-tert-butylphosphine)palladium(0) (0.2 g, 0.5 mmol) was added. After reacting for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound HE_1 (18.3 g). (Yield: 67%, MS: [M+H]+=567)

Compound HE_1 (15 g, 26.5 mmol) and phenylboronic acid (3.4 g, 27.8 mmol) were added to 1,4-dioxane (300 ml), and the mixture was stirred and refluxed. Then, potassium phosphate (16.9 g, 79.5 mmol) was dissolved in water (51 ml) and added thereto, and the mixture was sufficiently stirred, and then bis(tri-tert-butylphosphine)palladium(0) (0.1 g, 0.3 mmol) was added. After reacting for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 114 (11.4 g). (Yield: 71%, MS: [M+H]+=609)

### Example 115: Preparation of Compound 115

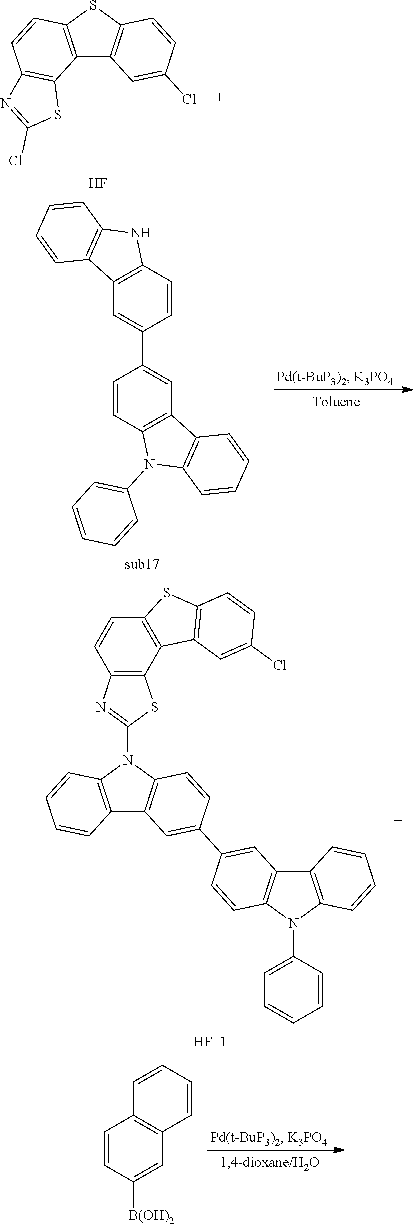

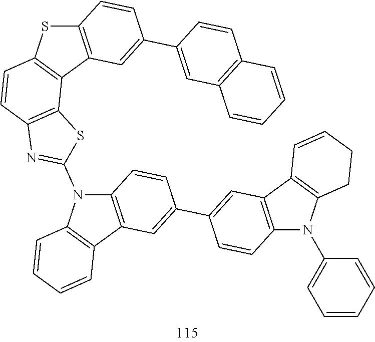 [SMILES: C1=Cc2c(n(-c3ccccc3)c3ccc(-c4ccc5c(c4)c4ccccc4n5-c4nc5ccc6sc7ccc(-c8ccc9ccccc9c8)cc7c6c5s4)cc23)CC1]

Compound HF (10 g, 32.2 mmol), sub17 (13.8 g, 33.8 mmol) and potassium phosphate (20.5 g, 96.7 mmol) were added to toluene (200 ml) under a nitrogen atmosphere, and the mixture was stirred and refluxed. Then, bis(tri-tert-butylphosphine)palladium(0) (0.2 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare Compound HF_1 (13.4 g). (Yield: 61%, MS: [M+H]+=682)

Compound HF_1 (15 g, 22 mmol) and naphthalen-2-ylboronic acid (4 g, 23.1 mmol) were added to 1,4-dioxane (300 ml), and the mixture was stirred and refluxed. Then, potassium phosphate (14 g, 66 mmol) was dissolved in water (42 ml) and added thereto, and the mixture was sufficiently stirred, and then bis(tri-tert-butylphosphine)palladium(0) (0.1 g, 0.2 mmol) was added. After reacting for 2 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give Compound 115 (12.1 g). (Yield: 71%, MS: [M+H]+=774)

### Experimental Examples

### Experimental Example 1

A glass substrate on which a thin film of ITO (indium tin oxide) was coated in a thickness of 1,000 Å was put into distilled water containing a detergent dissolved therein and ultrasonically washed. At this time, the detergent used was a product commercially available from Fischer Co. and the distilled water was one which had been twice filtered by using a filter commercially available from Millipore Co. After the ITO was cleaned for 30 minutes, ultrasonic cleaning was repeated twice using distilled water for 10 minutes. After the washing with distilled water was completed, the substrate was ultrasonically washed with solvents of isopropyl alcohol, acetone, and methanol, and dried, after which it was transported to a plasma cleaner. Then, the substrate was cleaned with oxygen plasma for 5 minutes, and then transferred to a vacuum evaporator.

On the ITO transparent electrode thus prepared, the following Compound HI-1 was formed in a thickness of 1150 Å as a hole injection layer, but the following Compound A-1 was p-doped at a concentration of 1.5 wt. %. The following Compound HT-1 was vacuum deposited on the hole injection layer to form a hole transport layer with a film thickness of 800 Å. Then, the following Compound EB-1 was vacuum deposited on the hole transport layer to a film thickness of 150 Å to form an electron blocking layer. Then, the above-mentioned Compound 1, and the following Compound Dp-7 were vacuum deposited in a weight ratio of 98:2 on the EB-1 deposited film to form a red light emitting layer with a film thickness of 400 Å. The following Compound HB-1 was vacuum deposited on the light emitting layer to a film thickness of 30 Å to form a hole blocking layer. The following Compound ET-1 and the following Compound LiQ were vacuum deposited in a weight ratio of 2:1 on the hole blocking layer to form an electron injection and transport layer with a film thickness of 300 Å. Lithium fluoride (LiF) and aluminum were sequentially deposited to have a thickness of 12 Å and 1,000 Å, respectively, on the electron injection and transport layer, thereby forming a cathode.

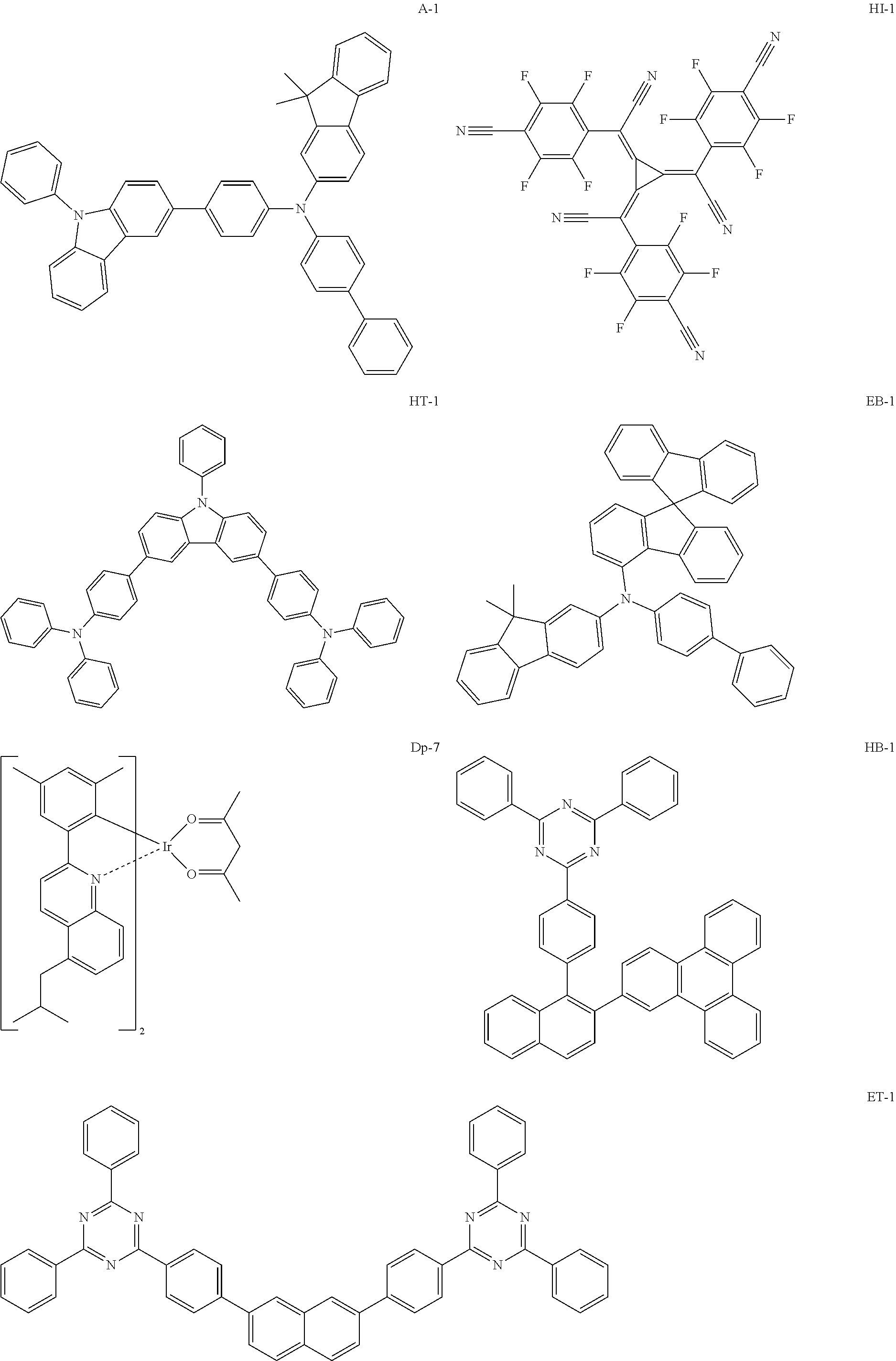

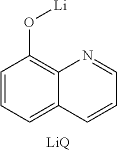

In the above-mentioned process, the vapor deposition rate of the organic material was maintained at 0.4-0.7 Å/sec, the deposition rates of lithium fluoride and the aluminum of the cathode were maintained at 0.3 Å/sec and 2 Å/sec, respectively, and the degree of vacuum during the deposition was maintained at 2×10⁻⁷˜5×10⁻⁶ torr, thereby manufacturing an organic light emitting device.

### Experimental Examples 2 to 115

The organic light emitting devices were manufactured in the same manner as in Experimental Example 1, except that the Compounds shown in Table 1 below were used instead of the Compound 1.

### Comparative Experimental Examples 1 to 16

The organic light emitting devices were manufactured in the same manner as in Experimental Example 1, except that the Comparative Compounds A-1 to A-16 shown in Table 2 below were used instead of the Compound 1. The Compounds A-1 to A-16 used in Table 2 are as follows.

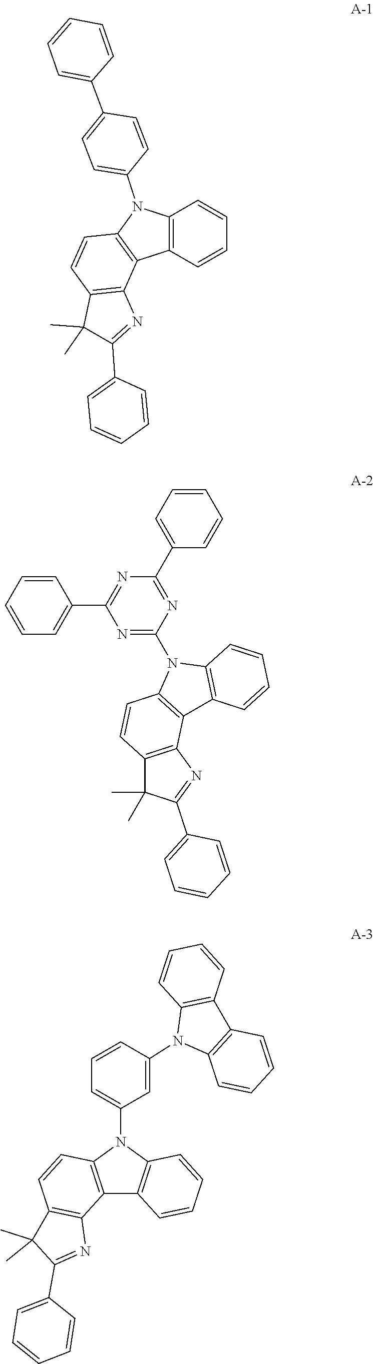 [SMILES: CC1(C)C(c2ccccc2)=Nc2c1ccc1c2c2ccccc2n1-c1ccc(-c2ccccc2)cc1 ; CC1(C)C(c2ccccc2)=Nc2c1ccc1c2c2ccccc2n1-c1nc(-c2ccccc2)nc(-c2ccccc2)n1 ; CC1(C)C(c2ccccc2)=Nc2c1ccc1c2c2ccccc2n1-c1cccc(-n2c3ccccc3c3ccccc32)c1]

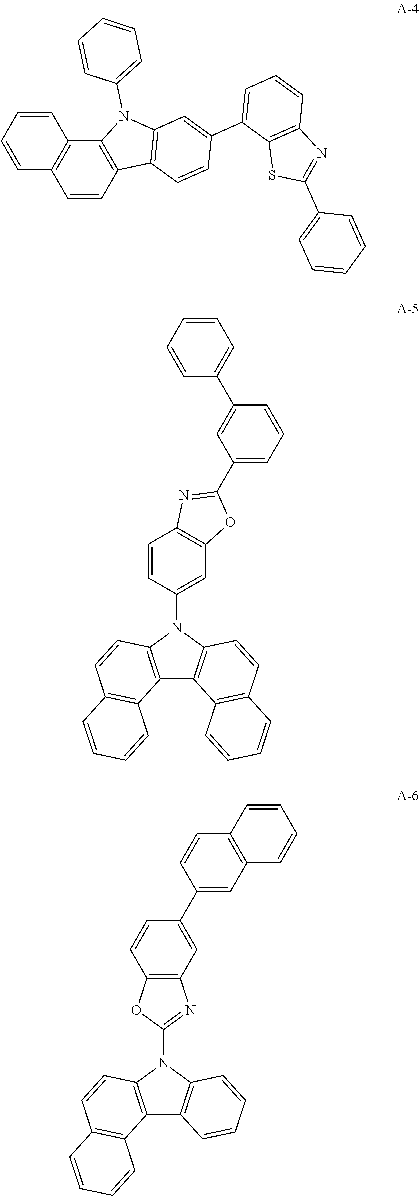 [SMILES: c1ccc(-c2nc3cccc(-c4ccc5c6ccc7ccccc7c6n(-c6ccccc6)c5c4)c3s2)cc1 ; c1ccc(-c2cccc(-c3nc4ccc(-n5c6ccc7ccccc7c6c6c7ccccc7ccc65)cc4o3)c2)cc1 ; c1ccc2cc(-c3ccc4oc(-n5c6ccccc6c6c7ccccc7ccc65)nc4c3)ccc2c1]

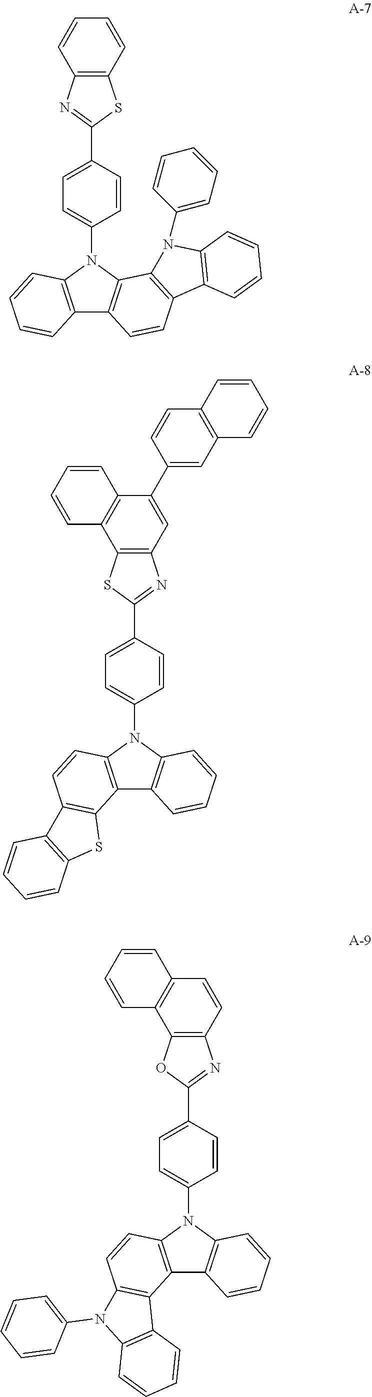 [SMILES: c1ccc(-n2c3ccccc3c3ccc4c5ccccc5n(-c5ccc(-c6nc7ccccc7s6)cc5)c4c32)cc1 ; c1ccc2cc(-c3cc4nc(-c5ccc(-n6c7ccccc7c7c8sc9ccccc9c8ccc76)cc5)sc4c4ccccc34)ccc2c1 ; c1ccc(-n2c3ccccc3c3c4c5ccccc5n(-c5ccc(-c6nc7ccc8ccccc8c7o6)cc5)c4ccc32)cc1]

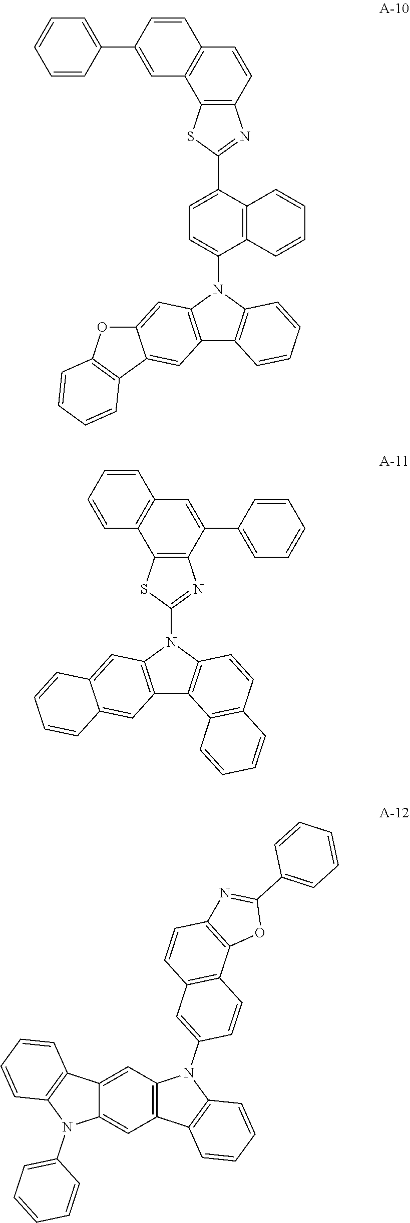 [SMILES: c1ccc(-c2ccc3ccc4nc(-c5ccc(-n6c7ccccc7c7cc8c(cc76)oc6ccccc68)c6ccccc56)sc4c3c2)cc1 ; c1ccc(-c2cc3ccccc3c3sc(-n4c5cc6ccccc6cc5c5c6ccccc6ccc54)nc23)cc1 ; c1ccc(-c2nc3ccc4cc(-n5c6ccccc6c6cc7c(cc65)c5ccccc5n7-c5ccccc5)ccc4c3o2)cc1]

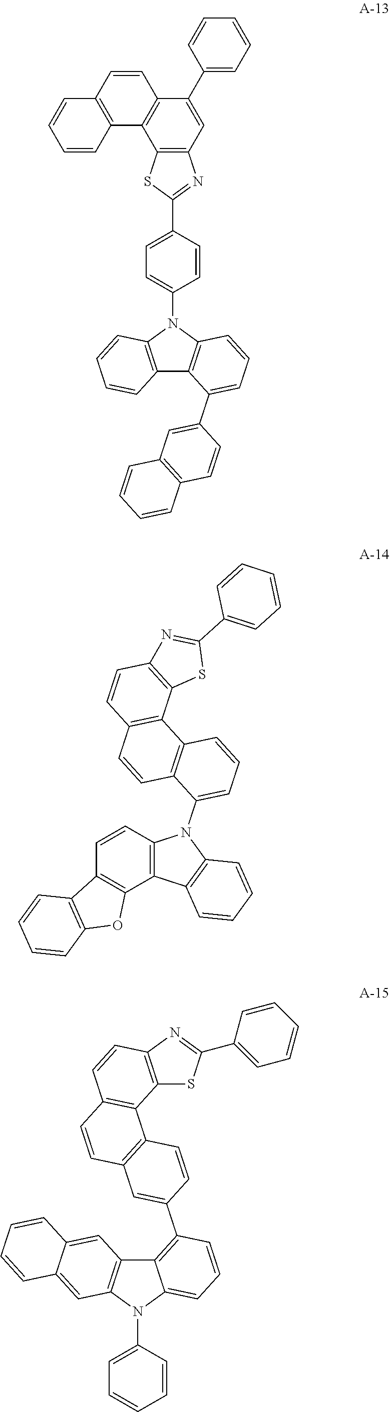 [SMILES: c1ccc(-c2cc3nc(-c4ccc(-n5c6ccccc6c6c(-c7ccc8ccccc8c7)cccc65)cc4)sc3c3c2ccc2ccccc23)cc1 ; c1ccc(-c2nc3ccc4ccc5c(-n6c7ccccc7c7c8oc9ccccc9c8ccc76)cccc5c4c3s2)cc1 ; c1ccc(-c2nc3ccc4ccc5cc(-c6cccc7c6c6cc8ccccc8cc6n7-c6ccccc6)ccc5c4c3s2)cc1]

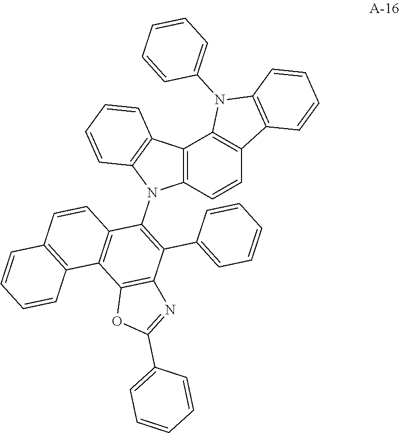 [SMILES: c1ccc(-c2nc3c(-c4ccccc4)c(-n4c5ccccc5c5c4ccc4c6ccccc6n(-c6ccccc6)c45)c4ccc5ccccc5c4c3o2)cc1]

The voltage and efficiency were measured (based on 15 mA/cm²) by applying a current to the organic light emitting devices manufactured in Experimental Examples 1 to 115 and Comparative Experimental Examples 1 to 16, and the results are shown in Tables 1 and 2 below. Lifetime T95 was measured based on 7000 nits, and means the time required for the lifetime to be reduced to 95% of the initial lifetime.

TABLE 1

| | | Driving | | Lifetime | |
|---|---|---|---|---|---|
| | | voltage | Efficiency | T95 | Luminous |
| Category | Host | (V) | (cd/A) | (hr) | color |
| Experimental | Compound | 3.48 | 23.81 | 275 | Red |
| Example 1 | 1 | | | | |
| Experimental | Compound | 3.49 | 23.26 | 265 | Red |
| Example 2 | 2 | | | | |
| Experimental | Compound | 3.50 | 23.92 | 269 | Red |
| Example 3 | 3 | | | | |
| Experimental | Compound | 3.47 | 23.36 | 267 | Red |
| Example 4 | 4 | | | | |
| Experimental | Compound | 3.51 | 23.96 | 263 | Red |
| Example 5 | 5 | | | | |
| Experimental | Compound | 3.46 | 23.80 | 279 | Red |
| Example 6 | 6 | | | | |
| Experimental | Compound | 3.49 | 23.07 | 275 | Red |
| Example 7 | 7 | | | | |
| Experimental | Compound | 3.49 | 23.90 | 279 | Red |
| Example 8 | 8 | | | | |
| Experimental | Compound | 3.45 | 23.71 | 265 | Red |
| Example 9 | 9 | | | | |
| Experimental | Compound | 3.48 | 23.43 | 278 | Red |
| Example 10 | 10 | | | | |
| Experimental | Compound | 3.50 | 23.28 | 277 | Red |
| Example 11 | 11 | | | | |
| Experimental | Compound | 3.51 | 22.98 | 266 | Red |
| Example 12 | 12 | | | | |
| Experimental | Compound | 3.54 | 23.35 | 276 | Red |
| Example 13 | 13 | | | | |
| Experimental | Compound | 3.54 | 23.61 | 278 | Red |
| Example 14 | 14 | | | | |
| Experimental | Compound | 3.54 | 23.48 | 266 | Red |
| Example 15 | 15 | | | | |
| Experimental | Compound | 3.54 | 23.63 | 260 | Red |
| Example 16 | 16 | | | | |
| Experimental | Compound | 3.50 | 23.27 | 263 | Red |
| Example 17 | 17 | | | | |
| Experimental | Compound | 3.46 | 23.10 | 268 | Red |
| Example 18 | 18 | | | | |
| Experimental | Compound | 3.45 | 23.76 | 266 | Red |
| Example 19 | 19 | | | | |
| Experimental | Compound | 3.47 | 22.66 | 246 | Red |
| Example 20 | 20 | | | | |
| Experimental | Compound | 3.53 | 22.86 | 247 | Red |
| Example 21 | 21 | | | | |
| Experimental | Compound | 3.50 | 22.51 | 243 | Red |
| Example 22 | 22 | | | | |
| Experimental | Compound | 3.52 | 22.71 | 246 | Red |
| Example 23 | 23 | | | | |
| Experimental | Compound | 3.51 | 22.63 | 249 | Red |
| Example 24 | 24 | | | | |
| Experimental | Compound | 3.50 | 22.81 | 246 | Red |
| Example 25 | 25 | | | | |
| Experimental | Compound | 3.50 | 22.71 | 240 | Red |
| Example 26 | 26 | | | | |
| Experimental | Compound | 3.51 | 22.76 | 260 | Red |
| Example 27 | 27 | | | | |
| Experimental | Compound | 3.48 | 22.62 | 244 | Red |
| Example 28 | 28 | | | | |
| Experimental | Compound | 3.53 | 22.89 | 262 | Red |
| Example 29 | 29 | | | | |
| Experimental | Compound | 3.57 | 12.16 | 248 | Red |
| Example 30 | 30 | | | | |
| Experimental | Compound | 3.45 | 22.52 | 254 | Red |
| Example 31 | 31 | | | | |
| Experimental | Compound | 3.52 | 22.86 | 243 | Red |
| Example 32 | 32 | | | | |
| Experimental | Compound | 3.54 | 22.83 | 252 | Red |
| Example 33 | 33 | | | | |
| Experimental | Compound | 3.58 | 22.67 | 276 | Red |
| Example 34 | 34 | | | | |
| Experimental | Compound | 3.54 | 22.89 | 265 | Red |
| Example 35 | 35 | | | | |
| Experimental | Compound | 3.52 | 22.74 | 261 | Red |
| Example 36 | 36 | | | | |
| Experimental | Compound | 3.57 | 22.74 | 268 | Red |
| Example 37 | 37 | | | | |
| Experimental | Compound | 3.59 | 22.58 | 262 | Red |
| Example 38 | 38 | | | | |
| Experimental | Compound | 3.53 | 22.72 | 280 | Red |
| Example 39 | 39 | | | | |
| Experimental | Compound | 3.54 | 22.61 | 277 | Red |
| Example 40 | 40 | | | | |
| Experimental | Compound | 3.54 | 22.51 | 275 | Red |
| Example 41 | 41 | | | | |
| Experimental | Compound | 3.56 | 22.73 | 274 | Red |
| Example 42 | 42 | | | | |
| Experimental | Compound | 3.56 | 22.54 | 262 | Red |
| Example 43 | 43 | | | | |
| Experimental | Compound | 3.52 | 22.83 | 277 | Red |
| Example 44 | 44 | | | | |
| Experimental | Compound | 3.53 | 22.89 | 268 | Red |
| Example 45 | 45 | | | | |
| Experimental | Compound | 3.61 | 22.87 | 273 | Red |
| Example 46 | 46 | | | | |
| Experimental | Compound | 3.58 | 22.68 | 270 | Red |
| Example 47 | 47 | | | | |
| Experimental | Compound | 3.60 | 22.61 | 260 | Red |
| Example 48 | 48 | | | | |
| Experimental | Compound | 3.67 | 14.01 | 251 | Red |
| Example 49 | 49 | | | | |
| Experimental | Compound | 3.60 | 18.05 | 250 | Red |
| Example 50 | 50 | | | | |
| Experimental | Compound | 3.59 | 13.70 | 253 | Red |
| Example 51 | 51 | | | | |
| Experimental | Compound | 3.61 | 22.23 | 246 | Red |
| Example 52 | 52 | | | | |
| Experimental | Compound | 3.66 | 22.90 | 244 | Red |
| Example 53 | 53 | | | | |
| Experimental | Compound | 3.67 | 15.31 | 241 | Red |
| Example 54 | 54 | | | | |
| Experimental | Compound | 3.64 | 21.80 | 243 | Red |
| Example 55 | 55 | | | | |
| Experimental | Compound | 3.62 | 21.74 | 251 | Red |
| Example 56 | 56 | | | | |
| Experimental | Compound | 3.67 | 15.95 | 240 | Red |
| Example 57 | 57 | | | | |
| Experimental | Compound | 3.61 | 12.30 | 240 | Red |
| Example 58 | 58 | | | | |
| Experimental | Compound | 3.63 | 14.50 | 255 | Red |
| Example 59 | 59 | | | | |
| Experimental | Compound | 3.70 | 20.98 | 198 | Red |
| Example 60 | 60 | | | | |
| Experimental | Compound | 3.59 | 22.59 | 256 | Red |
| Example 61 | 61 | | | | |
| Experimental | Compound | 3.58 | 22.80 | 261 | Red |
| Example 62 | 62 | | | | |
| Experimental | Compound | 3.55 | 22.70 | 243 | Red |
| Example 63 | 63 | | | | |
| Experimental | Compound | 3.58 | 22.72 | 244 | Red |
| Example 64 | 64 | | | | |
| Experimental | Compound | 3.56 | 22.58 | 248 | Red |
| Example 65 | 65 | | | | |
| Experimental | Compound | 3.54 | 22.55 | 242 | Red |
| Example 66 | 66 | | | | |
| Experimental | Compound | 3.59 | 22.60 | 252 | Red |
| Example 67 | 67 | | | | |
| Experimental | Compound | 3.57 | 22.67 | 246 | Red |
| Example 68 | 68 | | | | |
| Experimental | Compound | 3.59 | 22.68 | 247 | Red |
| Example 69 | 69 | | | | |
| Experimental | Compound | 3.54 | 22.81 | 248 | Red |
| Example 70 | 70 | | | | |
| Experimental | Compound | 3.64 | 15.37 | 218 | Red |
| Example 71 | 71 | | | | |
| Experimental | Compound | 3.64 | 11.13 | 224 | Red |
| Example 72 | 72 | | | | |
| Experimental | Compound | 3.65 | 14.95 | 208 | Red |
| Example 73 | 73 | | | | |
| Experimental | Compound | 3.59 | 11.73 | 224 | Red |
| Example 74 | 74 | | | | |
| Experimental | Compound | 3.60 | 19.67 | 203 | Red |
| Example 75 | 75 | | | | |
| Experimental | Compound | 3.60 | 15.14 | 206 | Red |
| Example 76 | 76 | | | | |
| Experimental | Compound | 3.61 | 22.10 | 194 | Red |
| Example 77 | 77 | | | | |
| Experimental | Compound | 3.63 | 19.18 | 199 | Red |
| Example 78 | 78 | | | | |
| Experimental | Compound | 3.65 | 17.52 | 203 | Red |
| Example 79 | 79 | | | | |
| Experimental | Compound | 3.60 | 17.49 | 217 | Red |
| Example 80 | 80 | | | | |
| Experimental | Compound | 3.61 | 22.10 | 194 | Red |
| Example 81 | 81 | | | | |
| Experimental | Compound | 3.63 | 19.18 | 199 | Red |
| Example 82 | 82 | | | | |
| Experimental | Compound | 3.65 | 17.52 | 203 | Red |
| Example 83 | 83 | | | | |
| Experimental | Compound | 3.60 | 17.49 | 217 | Red |
| Example 84 | 84 | | | | |
| Experimental | Compound | 3.55 | 22.73 | 261 | Red |
| Example 85 | 85 | | | | |
| Experimental | Compound | 3.54 | 22.70 | 244 | Red |
| Example 86 | 86 | | | | |
| Experimental | Compound | 3.53 | 22.55 | 254 | Red |
| Example 87 | 87 | | | | |
| Experimental | Compound | 3.57 | 22.65 | 245 | Red |
| Example 88 | 88 | | | | |
| Experimental | Compound | 3.59 | 22.70 | 243 | Red |
| Example 89 | 89 | | | | |
| Experimental | Compound | 3.56 | 22.77 | 251 | Red |
| Example 90 | 90 | | | | |
| Experimental | Compound | 3.77 | 21.28 | 201 | Red |
| Example 91 | 91 | | | | |
| Experimental | Compound | 3.66 | 20.16 | 195 | Red |
| Example 92 | 92 | | | | |
| Experimental | Compound | 3.76 | 20.21 | 205 | Red |
| Example 93 | 93 | | | | |
| Experimental | Compound | 3.65 | 20.49 | 203 | Red |
| Example 94 | 94 | | | | |
| Experimental | Compound | 3.78 | 20.38 | 209 | Red |
| Example 95 | 95 | | | | |
| Experimental | Compound | 3.65 | 20.53 | 202 | Red |
| Example 96 | 96 | | | | |
| Experimental | Compound | 3.70 | 20.21 | 224 | Red |
| Example 97 | 97 | | | | |
| Experimental | Compound | 3.71 | 20.19 | 222 | Red |
| Example 98 | 98 | | | | |
| Experimental | Compound | 3.74 | 21.48 | 213 | Red |
| Example 99 | 99 | | | | |
| Experimental | Compound | 3.73 | 20.14 | 220 | Red |
| Example 100 | 100 | | | | |
| Experimental | Compound | 3.64 | 16.17 | 193 | Red |
| Example 101 | 101 | | | | |
| Experimental | Compound | 3.65 | 13.24 | 220 | Red |
| Example 102 | 102 | | | | |
| Experimental | Compound | 3.62 | 16.67 | 216 | Red |
| Example 103 | 103 | | | | |
| Experimental | Compound | 3.78 | 21.33 | 209 | Red |
| Example 104 | 104 | | | | |
| Experimental | Compound | 3.75 | 20.46 | 191 | Red |
| Example 105 | 105 | | | | |
| Experimental | Compound | 3.75 | 21.39 | 213 | Red |
| Example 106 | 106 | | | | |
| Experimental | Compound | 3.71 | 20.57 | 219 | Red |
| Example 107 | 107 | | | | |
| Experimental | Compound | 3.78 | 21.02 | 190 | Red |
| Example 108 | 108 | | | | |
| Experimental | Compound | 3.71 | 21.29 | 191 | Red |
| Example 109 | 109 | | | | |
| Experimental | Compound | 3.73 | 20.29 | 222 | Red |
| Example 110 | 110 | | | | |
| Experimental | Compound | 3.71 | 20.80 | 208 | Red |
| Example 111 | 111 | | | | |
| Experimental | Compound | 3.68 | 21.22 | 219 | Red |
| Example 112 | 112 | | | | |
| Experimental | Compound | 3.67 | 21.04 | 210 | Red |
| Example 113 | 113 | | | | |
| Experimental | Compound | 3.69 | 20.71 | 209 | Red |
| Example 114 | 114 | | | | |
| Experimental | Compound | 3.74 | 21.25 | 209 | Red |
| Example 115 | 115 | | | | |

TABLE 2

| | | Driving | | Lifetime | |
|---|---|---|---|---|---|
| | | voltage | Efficiency | T95 | Luminous |
| Category | Host | (V) | (cd/A) | (hr) | color |
| Comparative | Compound | 4.09 | 13.53 | 47 | Red |
| Experimental | A-1 | | | | |
| Example 1 | | | | | |
| Comparative | Compound | 4.14 | 13.23 | 81 | Red |
| Experimental | A-2 | | | | |
| Example 2 | | | | | |
| Comparative | Compound | 4.22 | 12.83 | 64 | Red |
| Experimental | A-3 | | | | |
| Example 3 | | | | | |
| Comparative | Compound | 3.96 | 16.20 | 89 | Red |
| Experimental | A-4 | | | | |
| Example 4 | | | | | |
| Comparative | Compound | 3.89 | 13.14 | 74 | Red |
| Experimental | A-5 | | | | |
| Example 5 | | | | | |
| Comparative | Compound | 3.85 | 16.14 | 81 | Red |
| Experimental | A-6 | | | | |
| Example 6 | | | | | |
| Comparative | Compound | 3.94 | 15.81 | 92 | Red |
| Experimental | A-7 | | | | |
| Example 7 | | | | | |
| Comparative | Compound | 3.94 | 16.59 | 136 | Red |
| Experimental | A-8 | | | | |
| Example 8 | | | | | |
| Comparative | Compound | 3.91 | 17.11 | 148 | Red |
| Experimental | A-9 | | | | |
| Example 9 | | | | | |
| Comparative | Compound | 3.98 | 16.27 | 123 | Red |
| Experimental | A-10 | | | | |
| Example 10 | | | | | |
| Comparative | Compound | 3.93 | 16.79 | 94 | Red |
| Experimental | A-11 | | | | |
| Example 11 | | | | | |
| Comparative | Compound | 4.06 | 13.47 | 103 | Red |
| Experimental | A-12 | | | | |
| Example 12 | | | | | |
| Comparative | Compound | 3.89 | 15.80 | 122 | Red |
| Experimental | A-13 | | | | |
| Example 13 | | | | | |
| Comparative | Compound | 4.10 | 13.21 | 106 | Red |
| Experimental | A-14 | | | | |
| Example 14 | | | | | |
| Comparative | Compound | 4.13 | 13.49 | 77 | Red |
| Experimental | A-15 | | | | |
| Example 15 | | | | | |
| Comparative | Compound | 4.04 | 14.75 | 98 | Red |
| Experimental | A-16 | | | | |
| Example 16 | | | | | |

When a current was applied to the organic light emitting devices manufactured in Experimental Examples 1 to 115 and Comparative Experimental Examples 1 to 16, the results shown in Tables 1 and 2 were obtained.

The red organic light emitting device of Experimental Example 1 used a material widely used in the prior art, which has a structure in which the Compound [EB-1] was used as an electron blocking layer and Dp-7 was used as a dopant for the red light emitting layer. As shown in Table 2, when Compounds A-1 to A-16 of Comparative Experimental Examples were used, they generally resulted in an increase in the driving voltage and a decrease in the efficiency and lifetime as compared to the combination of the present disclosure. From the above results, it can be confirmed that the reason why the driving voltage is improved and the efficiency and lifetime are increased is that the compounds of the present disclosure have good energy transfer to the red dopant in the red light-emitting layer. As a result, it can be confirmed that the compounds of the present disclosure has a more stable balance in the light-emitting layer than the Comparative Compounds, whereby electrons and holes combine to form excitons, and the efficiency and lifetime are greatly increased.

In conclusion, it can be confirmed that when the compounds of the present disclosure were used as hosts for red light emitting layers, the driving voltage, luminous efficiency, and lifetime characteristics of the organic light-emitting device can be improved.

[Description of symbols]

| | |
|---|---|
| 1: substrate | 2: anode |
| 3: light emitting layer | 4: cathode |
| 5: hole injection layer | 6: hole transport layer |
| 7: electron blocking layer | 8: electron transport layer |
| 9: electron injection layer | |

1. A compound of the following Chemical Formula 1 or Chemical Formula 2: 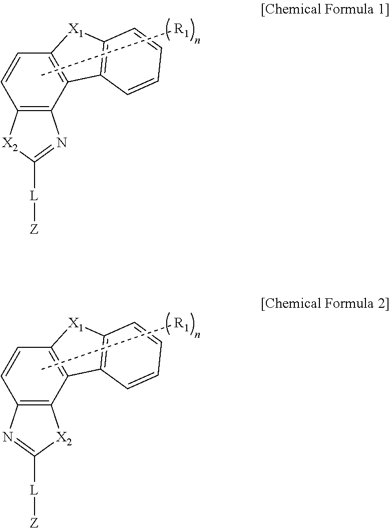
wherein, in Chemical Formulas 1 and 2;
X₁ and X₂ are each independently O or S;
each R₁ is independently hydrogen, deuterium, a substituted or unsubstituted C₆₋₆₀ aryl or a substituted or unsubstituted C₂₋₆₀ heteroaryl containing any one or more heteroatoms selected from the group consisting of N, O, and S;
L is a direct bond, a substituted or unsubstituted C₆₋₆₀ arylene, or a substituted or unsubstituted C₂₋₆₀ heteroarylene containing any one or more heteroatoms selected from the group consisting of N, O, and S; and
Z is the following Chemical Formula 3 or Chemical Formula 4: 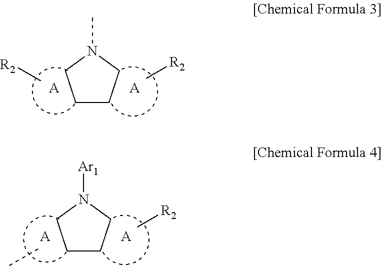
wherein, in Chemical Formulas 3 and 4:
each A is independently a substituted or unsubstituted C₆₋₆₀ aromatic ring fused with an adjacent five-membered ring, or a substituted or unsubstituted C₂₋₆₀ heteroaromatic ring containing any one or more heteroatoms selected from the group consisting of N, O, and S fused with an adjacent five-membered ring;
each R₂ is independently hydrogen, deuterium, a substituted or unsubstituted C₆₋₆₀ aryl, or a substituted or unsubstituted C₂₋₆₀ heteroaryl containing any one or more heteroatoms selected from the group consisting of N, O, and S;
Ar₁ is a substituted or unsubstituted C₆₋₆₀ aryl or a substituted or unsubstituted C₂₋₆₀ heteroaryl containing any one or more heteroatoms selected from the group consisting of N, O, and S; and
n is an integer of 0 to 6.

2. The compound of claim 1, wherein:
the Chemical Formula 1 is any one formula selected from the group consisting of the following Chemical Formulas 1-1 to 1-6, and the Chemical Formula 2 is any one formula selected from the group consisting of the following Chemical Formulas 2-1 to 2-6: 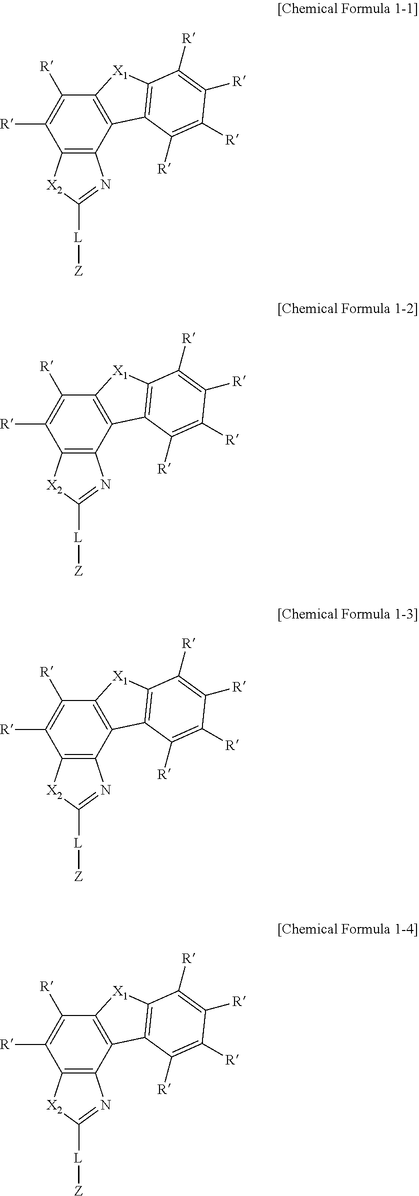 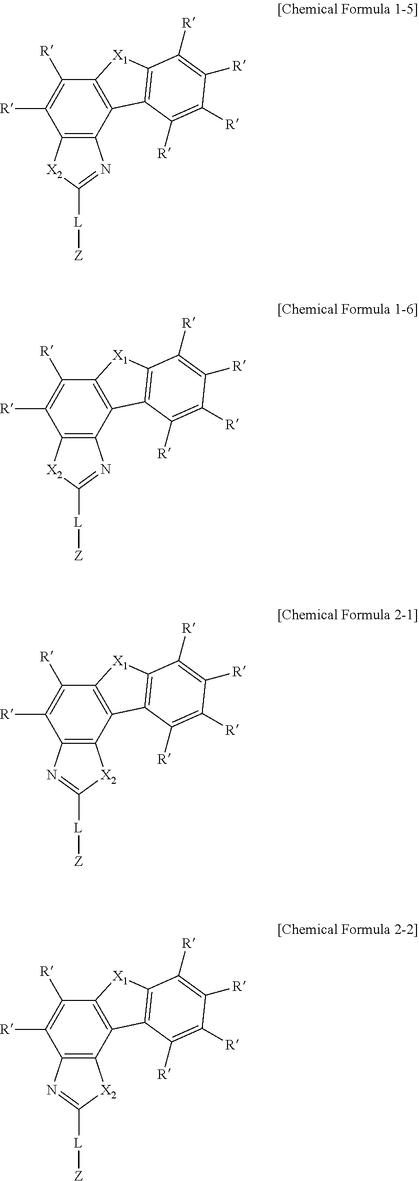 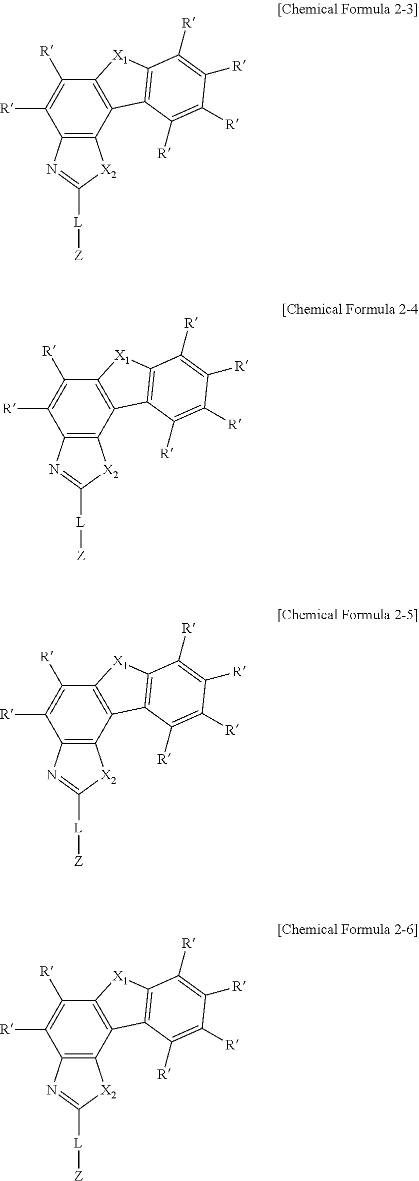
wherein, in Chemical Formulae 1-1 to 1-6 and Chemical Formulas 2-1 to 2-6:
X₁, X₂, L, and Z are as defined in claim 1;
each R₁ is independently a substituted or unsubstituted C₆₋₆₀ aryl or a substituted or unsubstituted C₂₋₆₀ heteroaryl containing any one or more heteroatoms selected from the group consisting of N, O, and S; and
each R′ is independently hydrogen or deuterium.

3. The compound of claim 1, wherein:
R₁ is hydrogen, deuterium, phenyl, biphenylyl, naphthyl, phenyl-naphthyl, naphthyl-phenyl, phenanthrenyl, or phenyl-phenanthrenyl.

4. The compound of claim 1, wherein:
L is a direct bond, or any one selected from the group of the following:

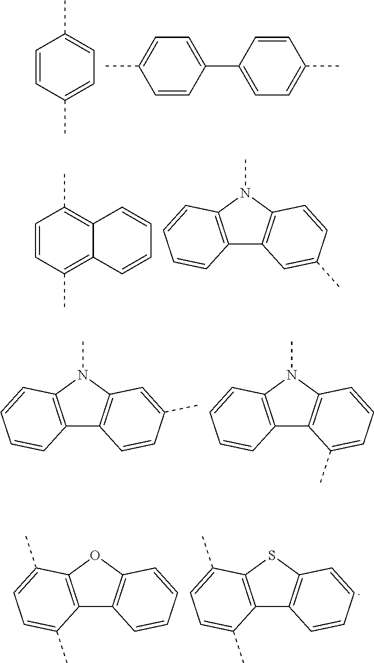

5. The compound of claim 1, wherein:
each A is independently benzene, naphthalene, carbazole, dibenzofuran, or dibenzothiophene.

6. The compound of claim 1, wherein:
each R₂ is independently hydrogen, deuterium, phenyl, naphthyl, 9-phenylcarbazolyl, carbazol-9-yl, or phenanthren-9-yl.

7. The compound of claim 1, wherein:
Ar₁ is phenyl, biphenylyl, naphthyl, phenyl-naphthyl, or naphthyl-phenyl.

8. The compound of claim 1, wherein:
the compound of Chemical Formula 1 or Chemical Formula 2 is any one selected from the group consisting of the following compounds:

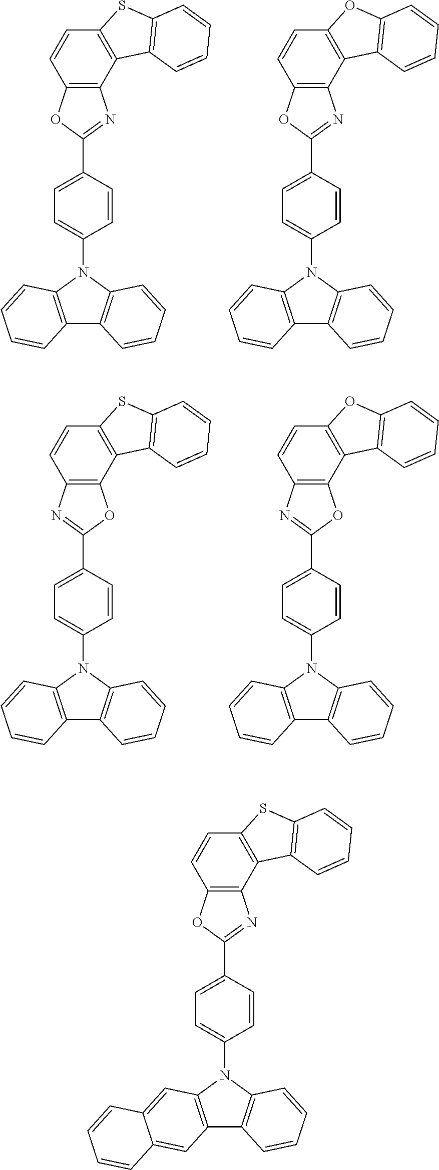 [SMILES: c1ccc2c(c1)sc1ccc3oc(-c4ccc(-n5c6ccccc6c6ccccc65)cc4)nc3c12 ; c1ccc2c(c1)oc1ccc3oc(-c4ccc(-n5c6ccccc6c6ccccc65)cc4)nc3c12 ; c1ccc2c(c1)sc1ccc3nc(-c4ccc(-n5c6ccccc6c6ccccc65)cc4)oc3c12 ; c1ccc2c(c1)oc1ccc3nc(-c4ccc(-n5c6ccccc6c6ccccc65)cc4)oc3c12 ; c1ccc2cc3c(cc2c1)c1ccccc1n3-c1ccc(-c2nc3c(ccc4sc5ccccc5c43)o2)cc1]

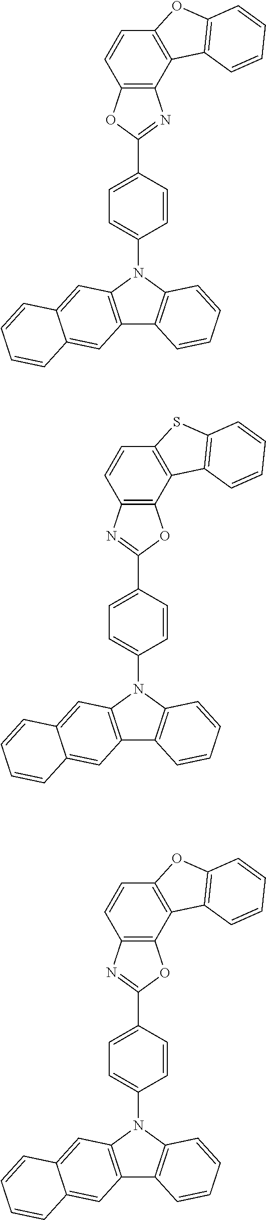 [SMILES: c1ccc2cc3c(cc2c1)c1ccccc1n3-c1ccc(-c2nc3c(ccc4oc5ccccc5c43)o2)cc1 ; c1ccc2cc3c(cc2c1)c1ccccc1n3-c1ccc(-c2nc3ccc4sc5ccccc5c4c3o2)cc1 ; c1ccc2cc3c(cc2c1)c1ccccc1n3-c1ccc(-c2nc3ccc4oc5ccccc5c4c3o2)cc1]

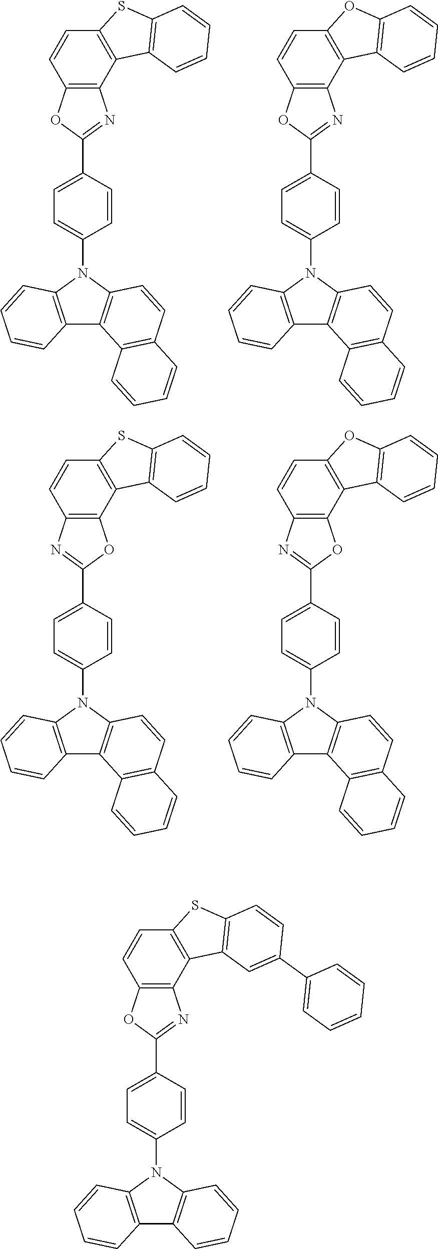

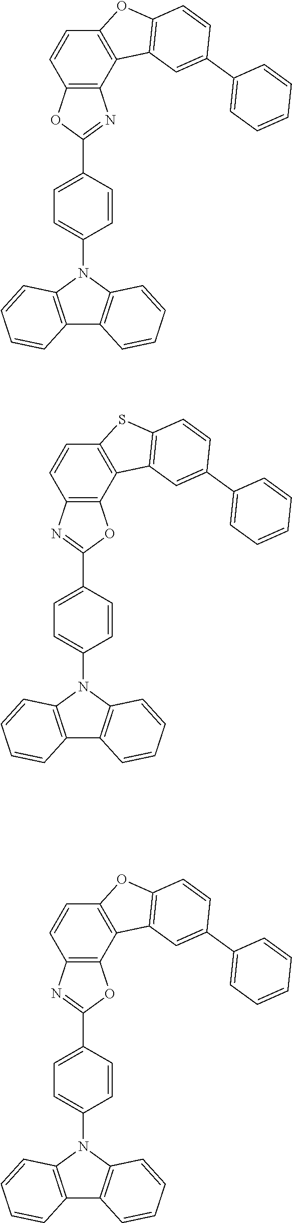 [SMILES: c1ccc(-c2ccc3oc4ccc5oc(-c6ccc(-n7c8ccccc8c8ccccc87)cc6)nc5c4c3c2)cc1 ; c1ccc(-c2ccc3sc4ccc5nc(-c6ccc(-n7c8ccccc8c8ccccc87)cc6)oc5c4c3c2)cc1 ; c1ccc(-c2ccc3oc4ccc5nc(-c6ccc(-n7c8ccccc8c8ccccc87)cc6)oc5c4c3c2)cc1]

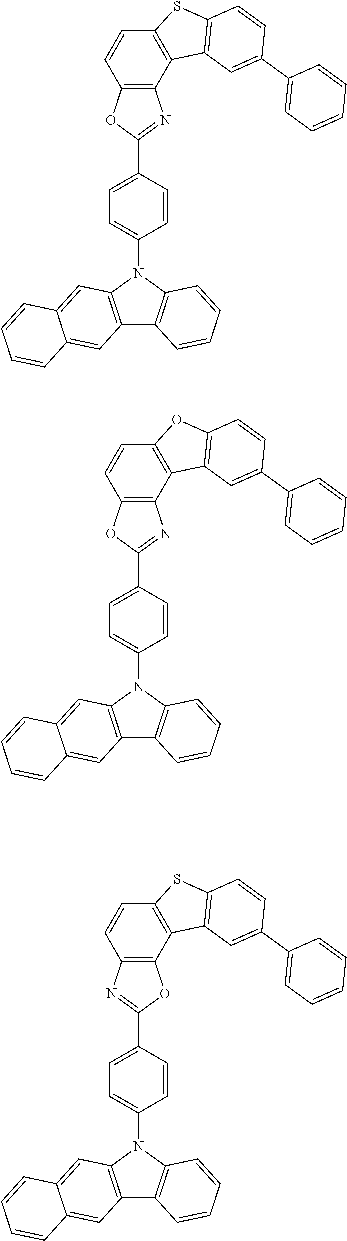 [SMILES: c1ccc(-c2ccc3sc4ccc5oc(-c6ccc(-n7c8ccccc8c8cc9ccccc9cc87)cc6)nc5c4c3c2)cc1 ; c1ccc(-c2ccc3oc4ccc5oc(-c6ccc(-n7c8ccccc8c8cc9ccccc9cc87)cc6)nc5c4c3c2)cc1 ; c1ccc(-c2ccc3sc4ccc5nc(-c6ccc(-n7c8ccccc8c8cc9ccccc9cc87)cc6)oc5c4c3c2)cc1]

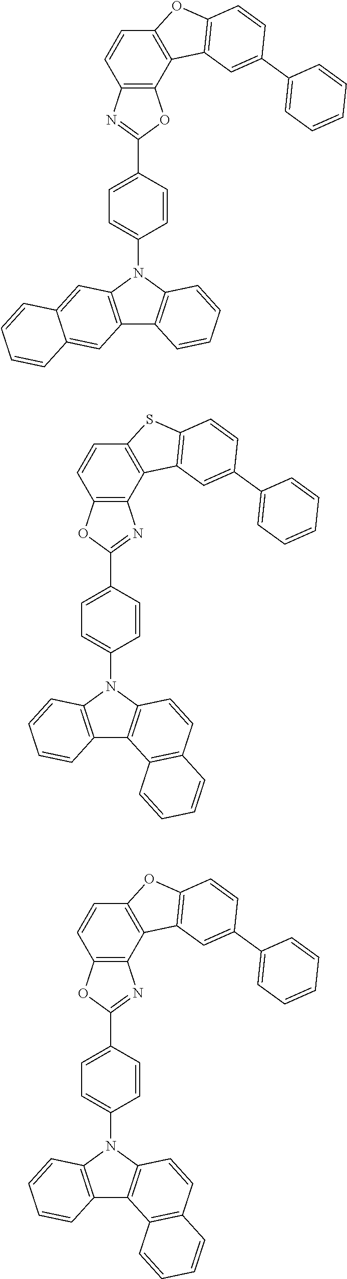 [SMILES: c1ccc(-c2ccc3oc4ccc5nc(-c6ccc(-n7c8ccccc8c8cc9ccccc9cc87)cc6)oc5c4c3c2)cc1 ; c1ccc(-c2ccc3sc4ccc5oc(-c6ccc(-n7c8ccccc8c8c9ccccc9ccc87)cc6)nc5c4c3c2)cc1 ; c1ccc(-c2ccc3oc4ccc5oc(-c6ccc(-n7c8ccccc8c8c9ccccc9ccc87)cc6)nc5c4c3c2)cc1]

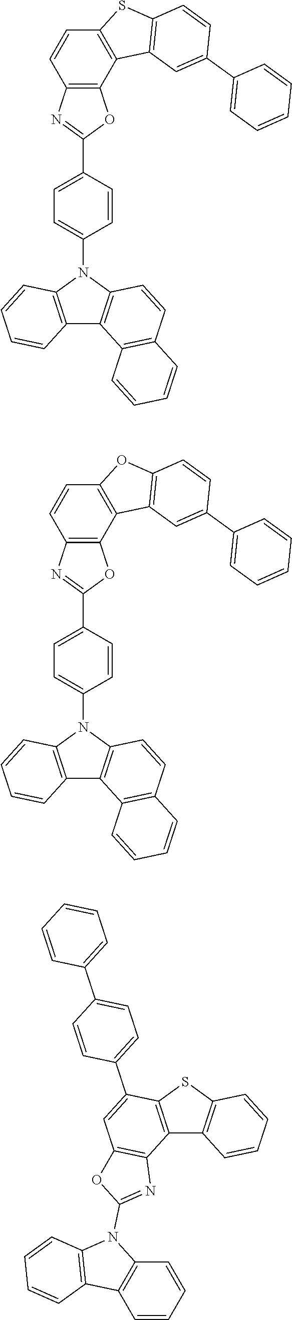 [SMILES: c1ccc(-c2ccc3sc4ccc5nc(-c6ccc(-n7c8ccccc8c8c9ccccc9ccc87)cc6)oc5c4c3c2)cc1 ; c1ccc(-c2ccc3oc4ccc5nc(-c6ccc(-n7c8ccccc8c8c9ccccc9ccc87)cc6)oc5c4c3c2)cc1 ; c1ccc(-c2ccc(-c3cc4oc(-n5c6ccccc6c6ccccc65)nc4c4c3sc3ccccc34)cc2)cc1]

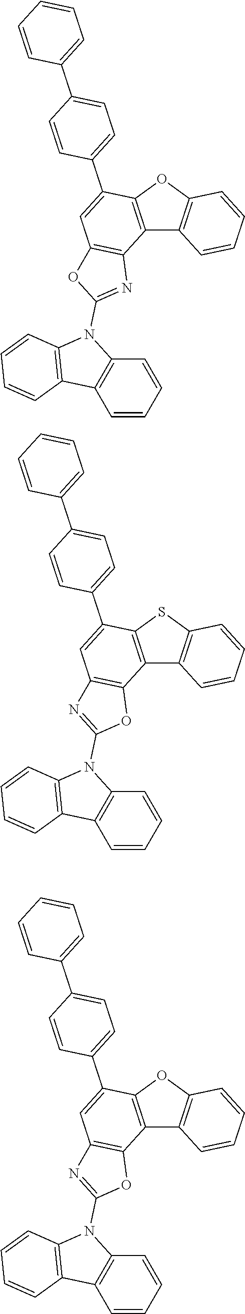

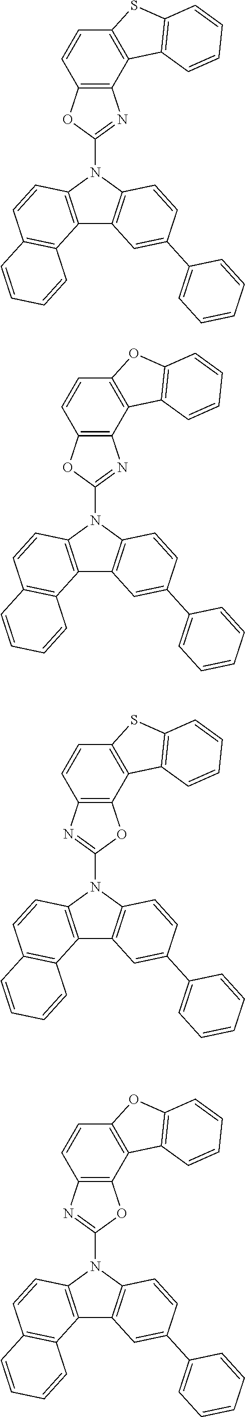

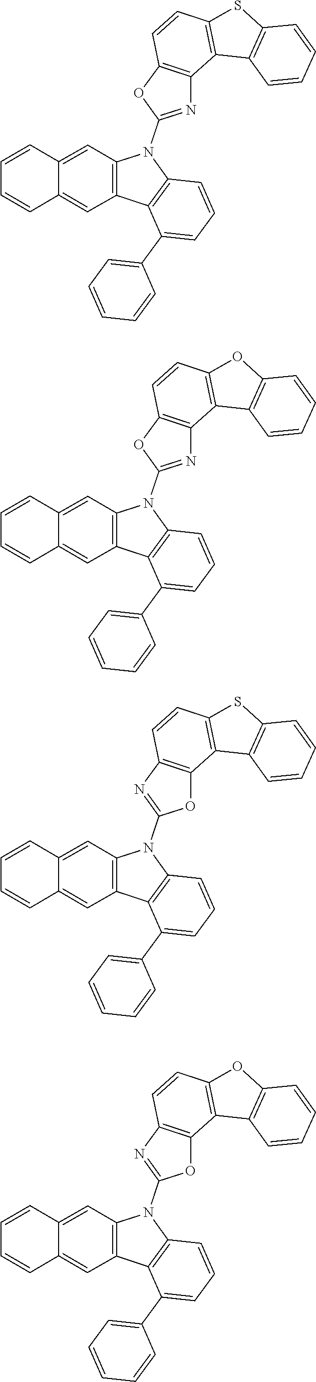 [SMILES: c1ccc(-c2cccc3c2c2cc4ccccc4cc2n3-c2nc3c(ccc4sc5ccccc5c43)o2)cc1 ; c1ccc(-c2cccc3c2c2cc4ccccc4cc2n3-c2nc3c(ccc4oc5ccccc5c43)o2)cc1 ; c1ccc(-c2cccc3c2c2cc4ccccc4cc2n3-c2nc3ccc4sc5ccccc5c4c3o2)cc1 ; c1ccc(-c2cccc3c2c2cc4ccccc4cc2n3-c2nc3ccc4oc5ccccc5c4c3o2)cc1]

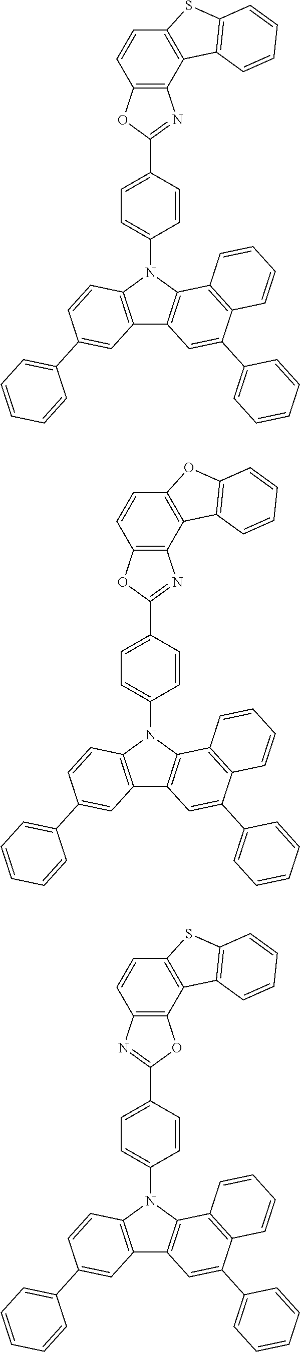

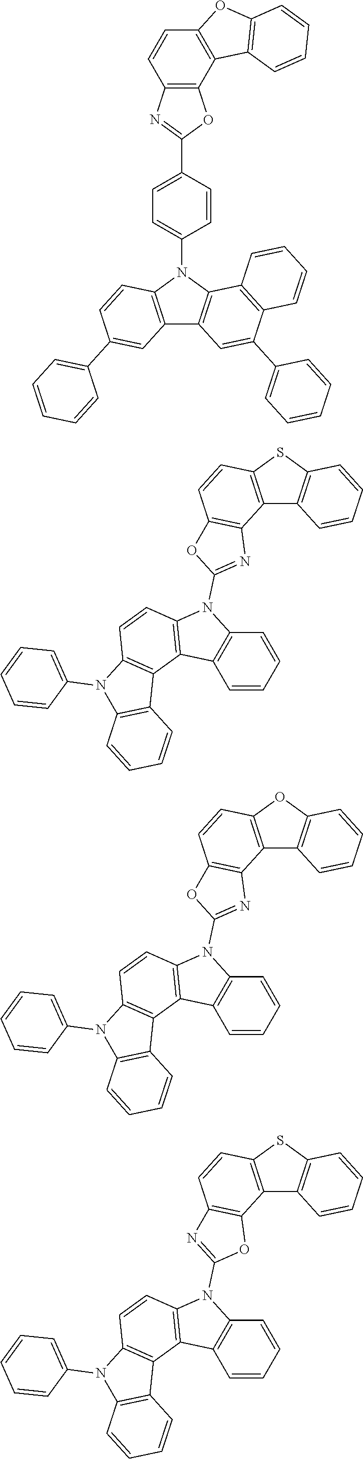

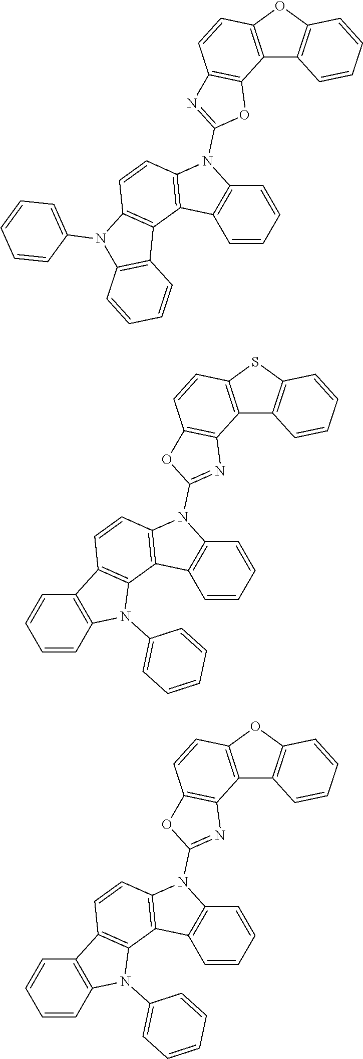 [SMILES: c1ccc(-n2c3ccccc3c3c4c5ccccc5n(-c5nc6ccc7oc8ccccc8c7c6o5)c4ccc32)cc1 ; c1ccc(-n2c3ccccc3c3ccc4c(c5ccccc5n4-c4nc5c(ccc6sc7ccccc7c65)o4)c32)cc1 ; c1ccc(-n2c3ccccc3c3ccc4c(c5ccccc5n4-c4nc5c(ccc6oc7ccccc7c65)o4)c32)cc1]

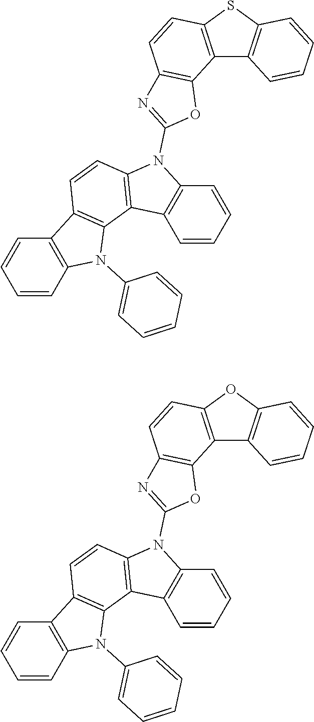 [SMILES: c1ccc(-n2c3ccccc3c3ccc4c(c5ccccc5n4-c4nc5ccc6sc7ccccc7c6c5o4)c32)cc1 ; c1ccc(-n2c3ccccc3c3ccc4c(c5ccccc5n4-c4nc5ccc6oc7ccccc7c6c5o4)c32)cc1]

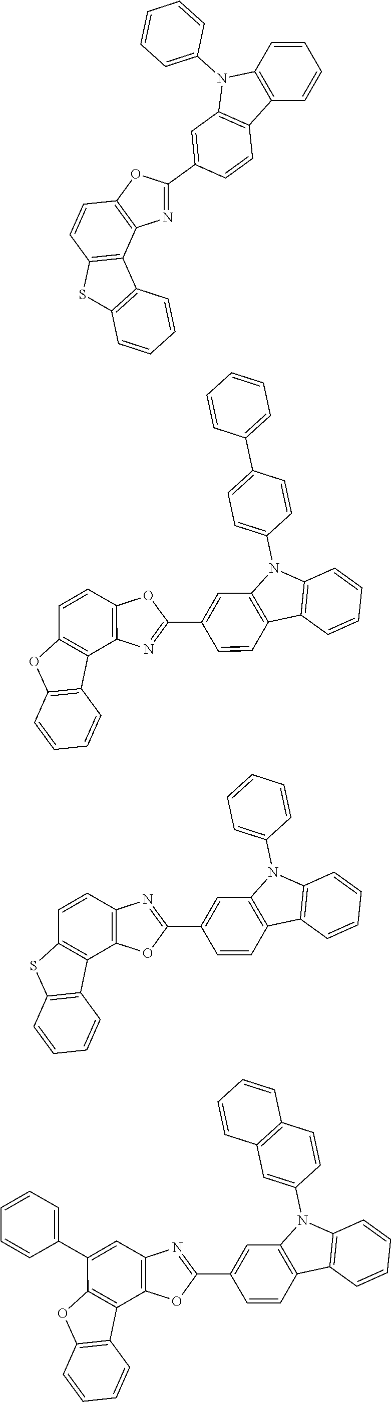 [SMILES: c1ccc(-n2c3ccccc3c3ccc(-c4nc5c(ccc6sc7ccccc7c65)o4)cc32)cc1 ; c1ccc(-c2ccc(-n3c4ccccc4c4ccc(-c5nc6c(ccc7oc8ccccc8c76)o5)cc43)cc2)cc1 ; c1ccc(-n2c3ccccc3c3ccc(-c4nc5ccc6sc7ccccc7c6c5o4)cc32)cc1 ; c1ccc(-c2cc3nc(-c4ccc5c6ccccc6n(-c6ccc7ccccc7c6)c5c4)oc3c3c2oc2ccccc23)cc1]

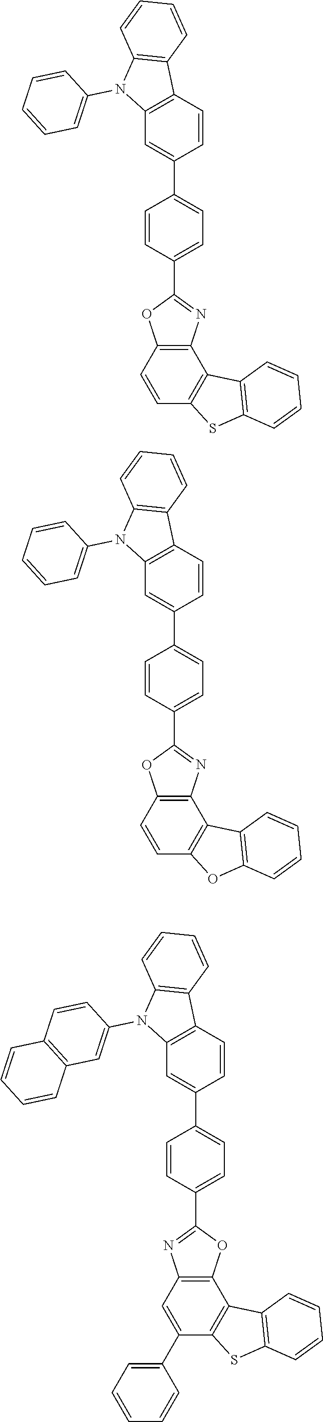 [SMILES: c1ccc(-n2c3ccccc3c3ccc(-c4ccc(-c5nc6c(ccc7sc8ccccc8c76)o5)cc4)cc32)cc1 ; c1ccc(-n2c3ccccc3c3ccc(-c4ccc(-c5nc6c(ccc7oc8ccccc8c76)o5)cc4)cc32)cc1 ; c1ccc(-c2cc3nc(-c4ccc(-c5ccc6c7ccccc7n(-c7ccc8ccccc8c7)c6c5)cc4)oc3c3c2sc2ccccc23)cc1]

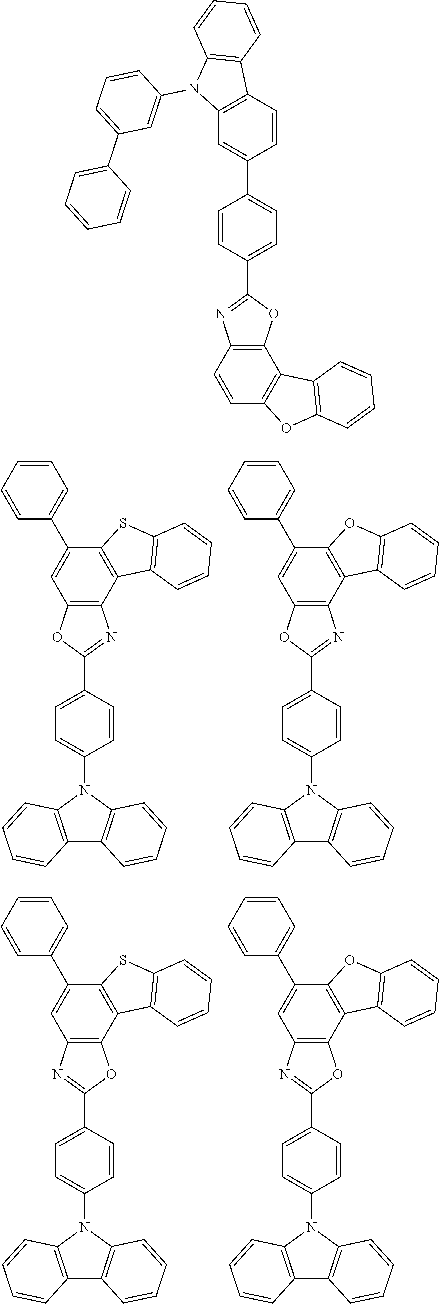

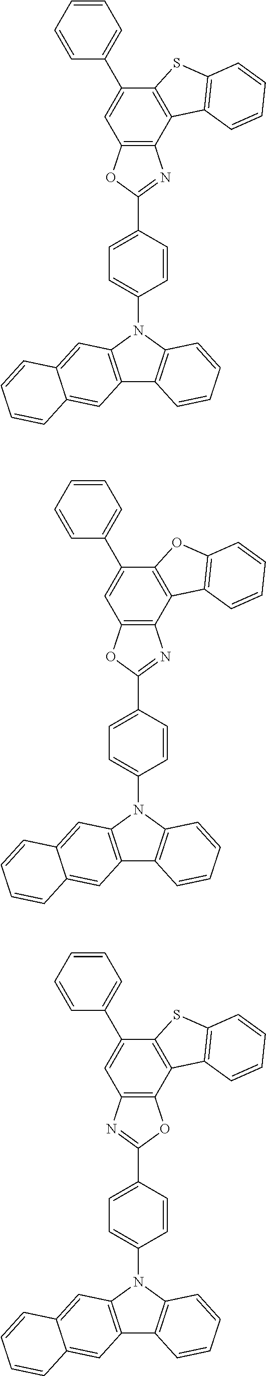 [SMILES: c1ccc(-c2cc3oc(-c4ccc(-n5c6ccccc6c6cc7ccccc7cc65)cc4)nc3c3c2sc2ccccc23)cc1 ; c1ccc(-c2cc3oc(-c4ccc(-n5c6ccccc6c6cc7ccccc7cc65)cc4)nc3c3c2oc2ccccc23)cc1 ; c1ccc(-c2cc3nc(-c4ccc(-n5c6ccccc6c6cc7ccccc7cc65)cc4)oc3c3c2sc2ccccc23)cc1]

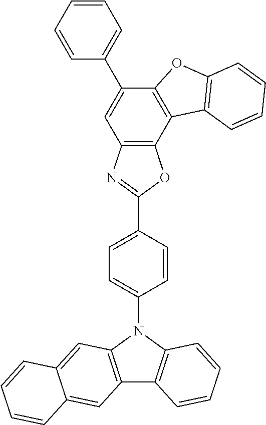 [SMILES: c1ccc(-c2cc3nc(-c4ccc(-n5c6ccccc6c6cc7ccccc7cc65)cc4)oc3c3c2oc2ccccc23)cc1]

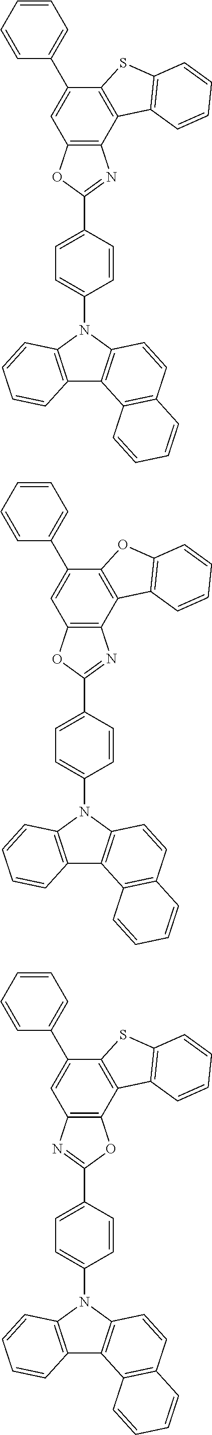 [SMILES: c1ccc(-c2cc3oc(-c4ccc(-n5c6ccccc6c6c7ccccc7ccc65)cc4)nc3c3c2sc2ccccc23)cc1 ; c1ccc(-c2cc3oc(-c4ccc(-n5c6ccccc6c6c7ccccc7ccc65)cc4)nc3c3c2oc2ccccc23)cc1 ; c1ccc(-c2cc3nc(-c4ccc(-n5c6ccccc6c6c7ccccc7ccc65)cc4)oc3c3c2sc2ccccc23)cc1]

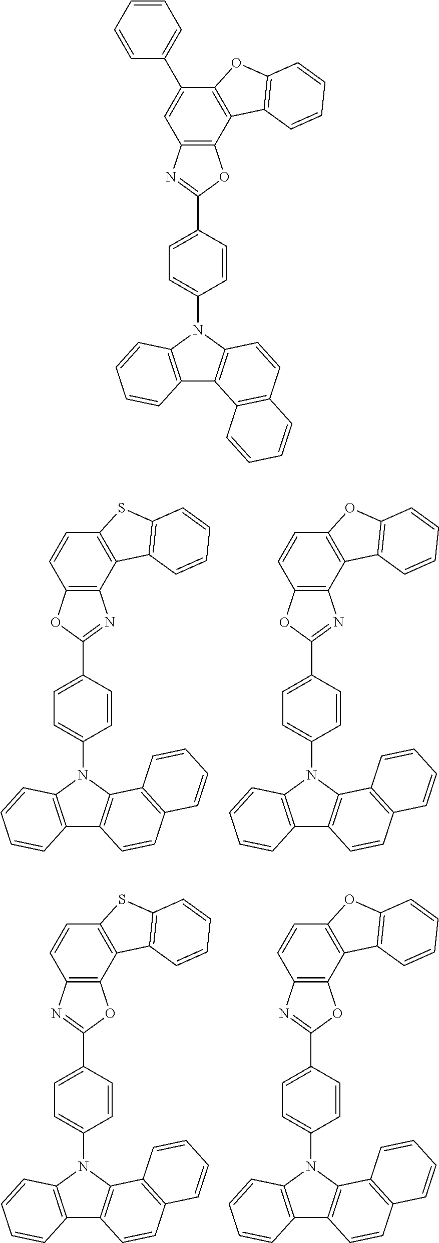

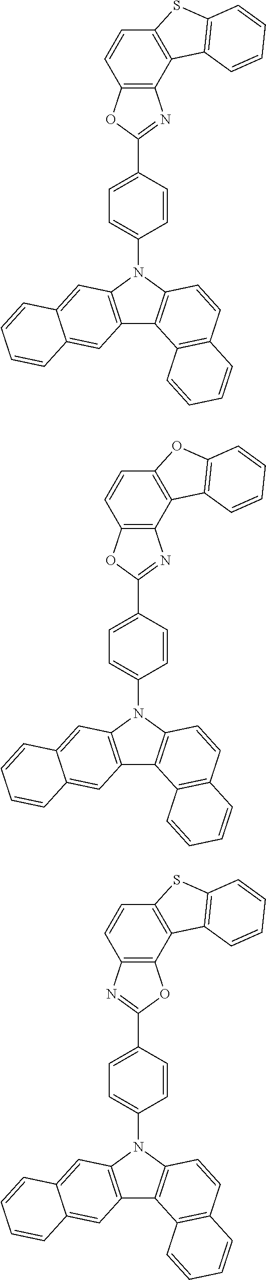

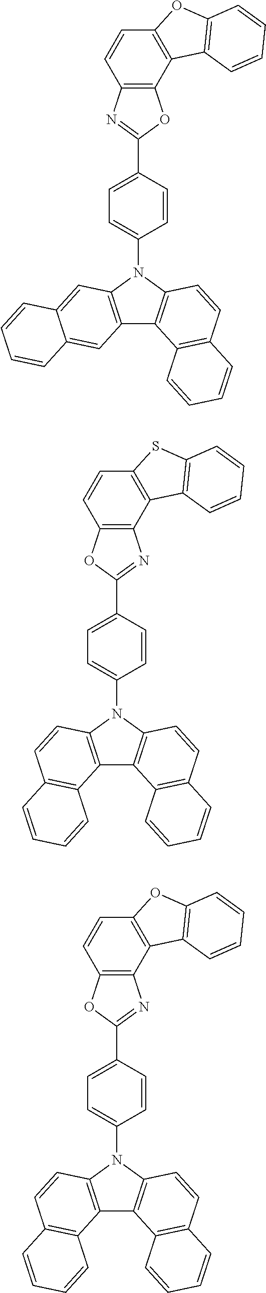 [SMILES: c1ccc2cc3c(cc2c1)c1c2ccccc2ccc1n3-c1ccc(-c2nc3ccc4oc5ccccc5c4c3o2)cc1 ; c1ccc2c(c1)ccc1c2c2c3ccccc3ccc2n1-c1ccc(-c2nc3c(ccc4sc5ccccc5c43)o2)cc1 ; c1ccc2c(c1)ccc1c2c2c3ccccc3ccc2n1-c1ccc(-c2nc3c(ccc4oc5ccccc5c43)o2)cc1]

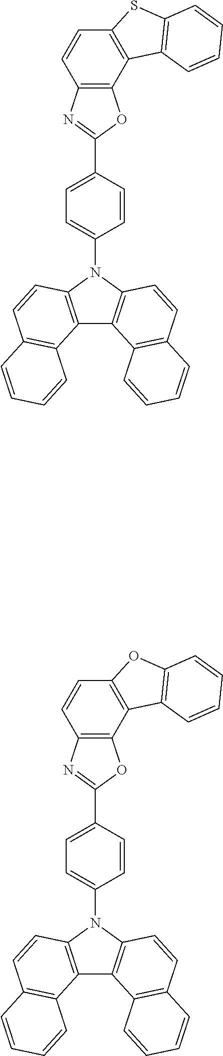 [SMILES: c1ccc2c(c1)ccc1c2c2c3ccccc3ccc2n1-c1ccc(-c2nc3ccc4sc5ccccc5c4c3o2)cc1 ; c1ccc2c(c1)ccc1c2c2c3ccccc3ccc2n1-c1ccc(-c2nc3ccc4oc5ccccc5c4c3o2)cc1]

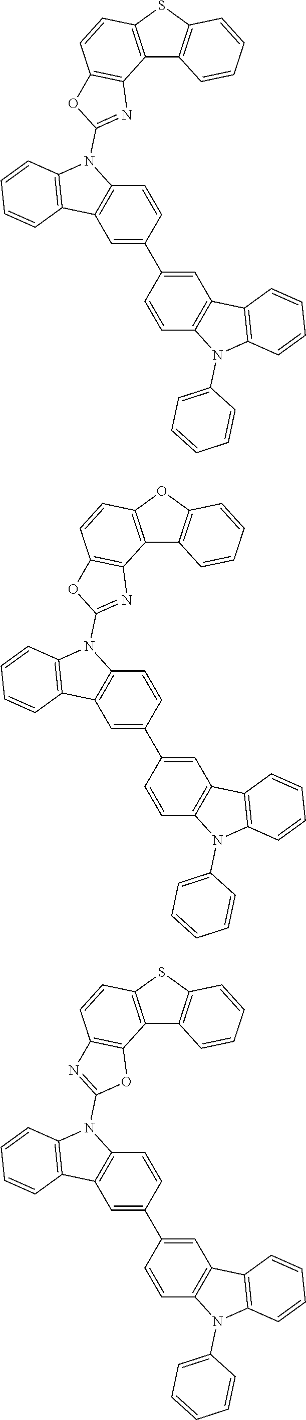 [SMILES: c1ccc(-n2c3ccccc3c3cc(-c4ccc5c(c4)c4ccccc4n5-c4nc5c(ccc6sc7ccccc7c65)o4)ccc32)cc1 ; c1ccc(-n2c3ccccc3c3cc(-c4ccc5c(c4)c4ccccc4n5-c4nc5c(ccc6oc7ccccc7c65)o4)ccc32)cc1 ; c1ccc(-n2c3ccccc3c3cc(-c4ccc5c(c4)c4ccccc4n5-c4nc5ccc6sc7ccccc7c6c5o4)ccc32)cc1]

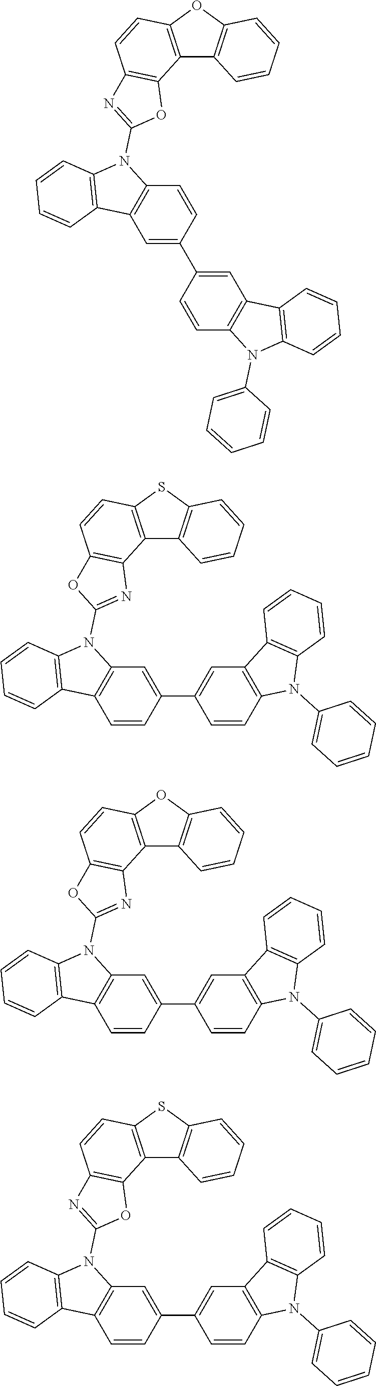 [SMILES: c1ccc(-n2c3ccccc3c3cc(-c4ccc5c(c4)c4ccccc4n5-c4nc5ccc6oc7ccccc7c6c5o4)ccc32)cc1 ; c1ccc(-n2c3ccccc3c3cc(-c4ccc5c6ccccc6n(-c6nc7c(ccc8sc9ccccc9c87)o6)c5c4)ccc32)cc1 ; c1ccc(-n2c3ccccc3c3cc(-c4ccc5c6ccccc6n(-c6nc7c(ccc8oc9ccccc9c87)o6)c5c4)ccc32)cc1 ; c1ccc(-n2c3ccccc3c3cc(-c4ccc5c6ccccc6n(-c6nc7ccc8sc9ccccc9c8c7o6)c5c4)ccc32)cc1]

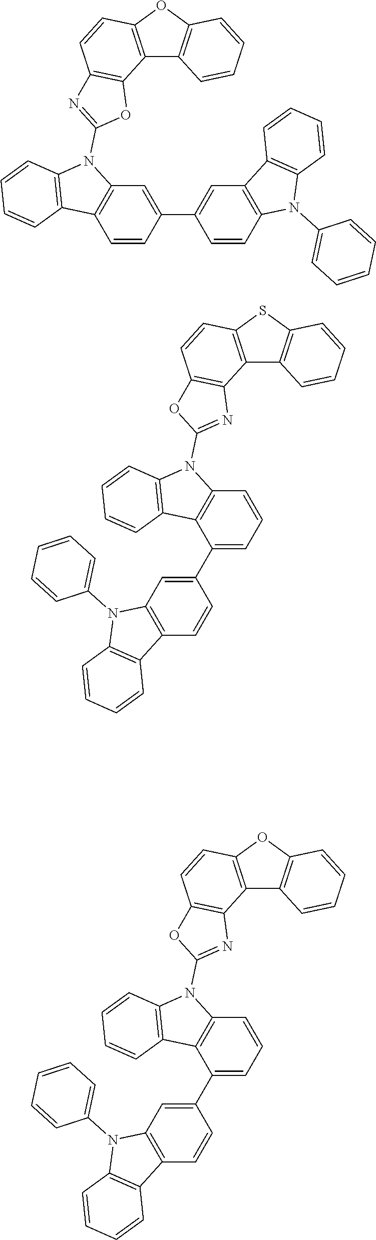

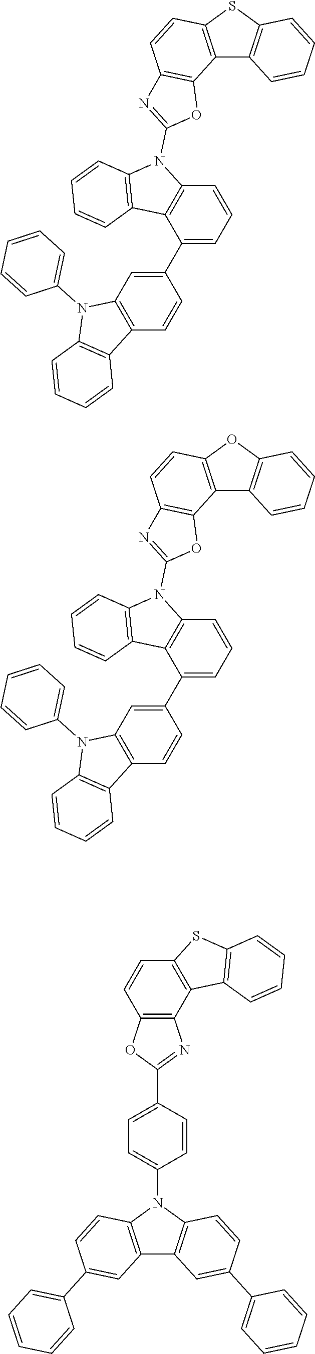 [SMILES: c1ccc(-n2c3ccccc3c3ccc(-c4cccc5c4c4ccccc4n5-c4nc5ccc6sc7ccccc7c6c5o4)cc32)cc1 ; c1ccc(-n2c3ccccc3c3ccc(-c4cccc5c4c4ccccc4n5-c4nc5ccc6oc7ccccc7c6c5o4)cc32)cc1 ; c1ccc(-c2ccc3c(c2)c2cc(-c4ccccc4)ccc2n3-c2ccc(-c3nc4c(ccc5sc6ccccc6c54)o3)cc2)cc1]

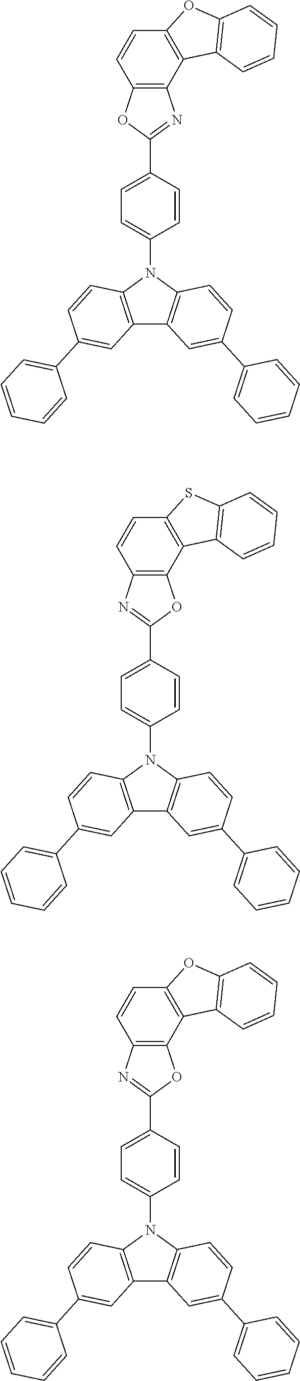 [SMILES: c1ccc(-c2ccc3c(c2)c2cc(-c4ccccc4)ccc2n3-c2ccc(-c3nc4c(ccc5oc6ccccc6c54)o3)cc2)cc1 ; c1ccc(-c2ccc3c(c2)c2cc(-c4ccccc4)ccc2n3-c2ccc(-c3nc4ccc5sc6ccccc6c5c4o3)cc2)cc1 ; c1ccc(-c2ccc3c(c2)c2cc(-c4ccccc4)ccc2n3-c2ccc(-c3nc4ccc5oc6ccccc6c5c4o3)cc2)cc1]

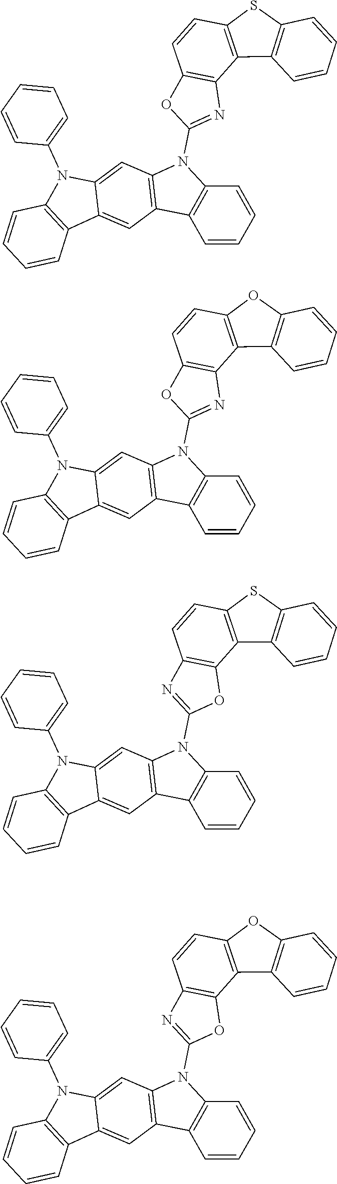 [SMILES: c1ccc(-n2c3ccccc3c3cc4c5ccccc5n(-c5nc6c(ccc7sc8ccccc8c76)o5)c4cc32)cc1 ; c1ccc(-n2c3ccccc3c3cc4c5ccccc5n(-c5nc6c(ccc7oc8ccccc8c76)o5)c4cc32)cc1 ; c1ccc(-n2c3ccccc3c3cc4c5ccccc5n(-c5nc6ccc7sc8ccccc8c7c6o5)c4cc32)cc1 ; c1ccc(-n2c3ccccc3c3cc4c5ccccc5n(-c5nc6ccc7oc8ccccc8c7c6o5)c4cc32)cc1]

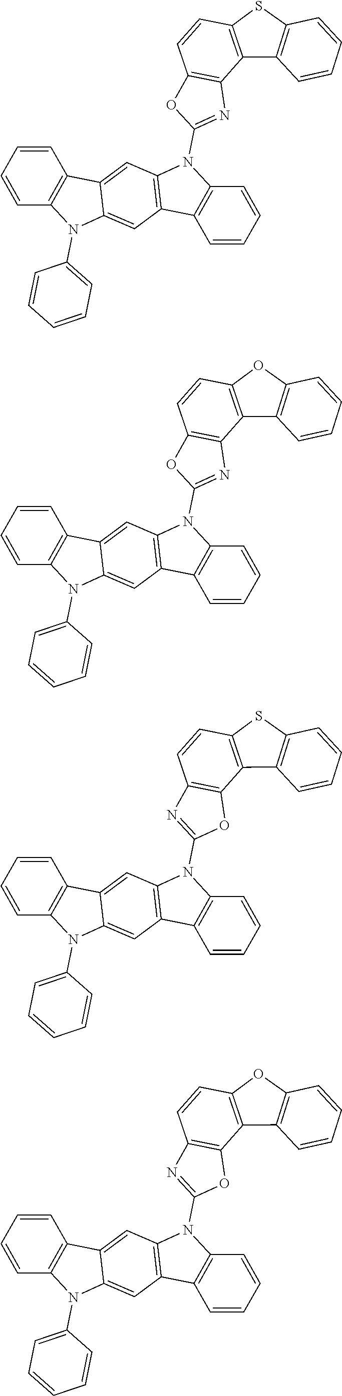 [SMILES: c1ccc(-n2c3ccccc3c3cc4c(cc32)c2ccccc2n4-c2nc3c(ccc4sc5ccccc5c43)o2)cc1 ; c1ccc(-n2c3ccccc3c3cc4c(cc32)c2ccccc2n4-c2nc3c(ccc4oc5ccccc5c43)o2)cc1 ; c1ccc(-n2c3ccccc3c3cc4c(cc32)c2ccccc2n4-c2nc3ccc4sc5ccccc5c4c3o2)cc1 ; c1ccc(-n2c3ccccc3c3cc4c(cc32)c2ccccc2n4-c2nc3ccc4oc5ccccc5c4c3o2)cc1]

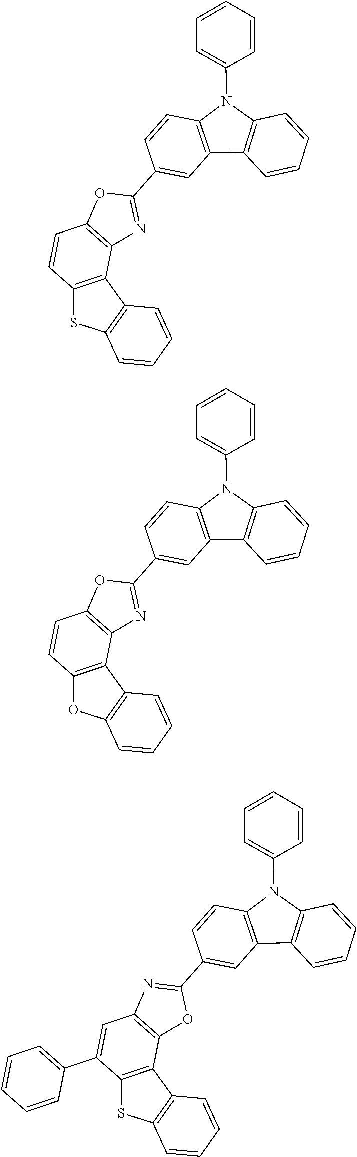 [SMILES: c1ccc(-n2c3ccccc3c3cc(-c4nc5c(ccc6sc7ccccc7c65)o4)ccc32)cc1 ; c1ccc(-n2c3ccccc3c3cc(-c4nc5c(ccc6oc7ccccc7c65)o4)ccc32)cc1 ; c1ccc(-c2cc3nc(-c4ccc5c(c4)c4ccccc4n5-c4ccccc4)oc3c3c2sc2ccccc23)cc1]

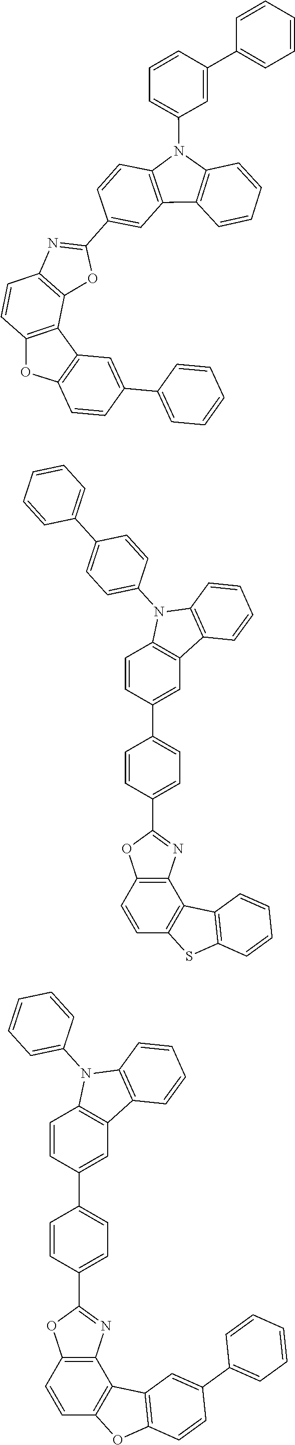 [SMILES: c1ccc(-c2cccc(-n3c4ccccc4c4cc(-c5nc6ccc7oc8ccc(-c9ccccc9)cc8c7c6o5)ccc43)c2)cc1 ; c1ccc(-c2ccc(-n3c4ccccc4c4cc(-c5ccc(-c6nc7c(ccc8sc9ccccc9c87)o6)cc5)ccc43)cc2)cc1 ; c1ccc(-c2ccc3oc4ccc5oc(-c6ccc(-c7ccc8c(c7)c7ccccc7n8-c7ccccc7)cc6)nc5c4c3c2)cc1]

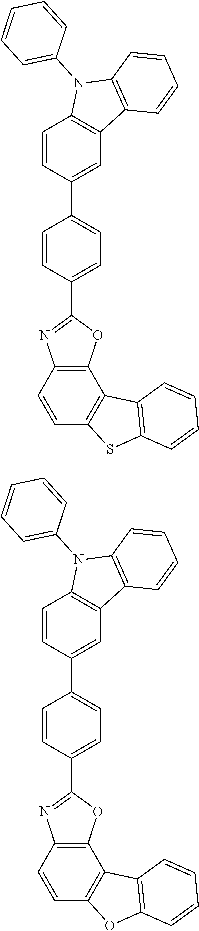 [SMILES: c1ccc(-n2c3ccccc3c3cc(-c4ccc(-c5nc6ccc7sc8ccccc8c7c6o5)cc4)ccc32)cc1 ; c1ccc(-n2c3ccccc3c3cc(-c4ccc(-c5nc6ccc7oc8ccccc8c7c6o5)cc4)ccc32)cc1]

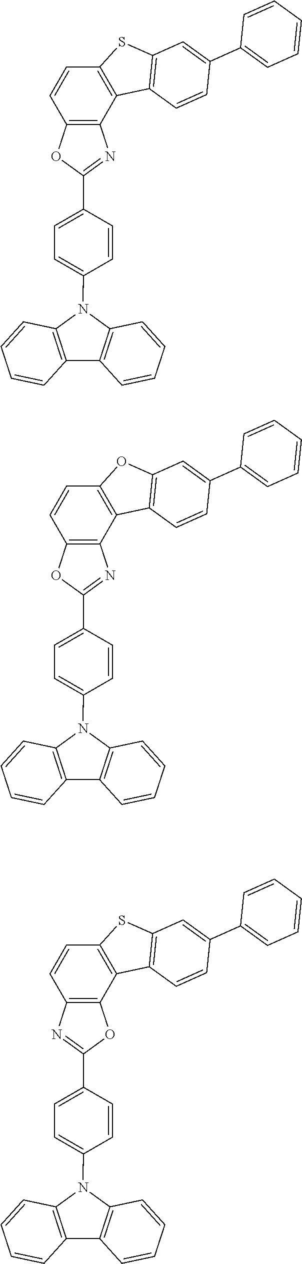 [SMILES: c1ccc(-c2ccc3c(c2)sc2ccc4oc(-c5ccc(-n6c7ccccc7c7ccccc76)cc5)nc4c23)cc1 ; c1ccc(-c2ccc3c(c2)oc2ccc4oc(-c5ccc(-n6c7ccccc7c7ccccc76)cc5)nc4c23)cc1 ; c1ccc(-c2ccc3c(c2)sc2ccc4nc(-c5ccc(-n6c7ccccc7c7ccccc76)cc5)oc4c23)cc1]

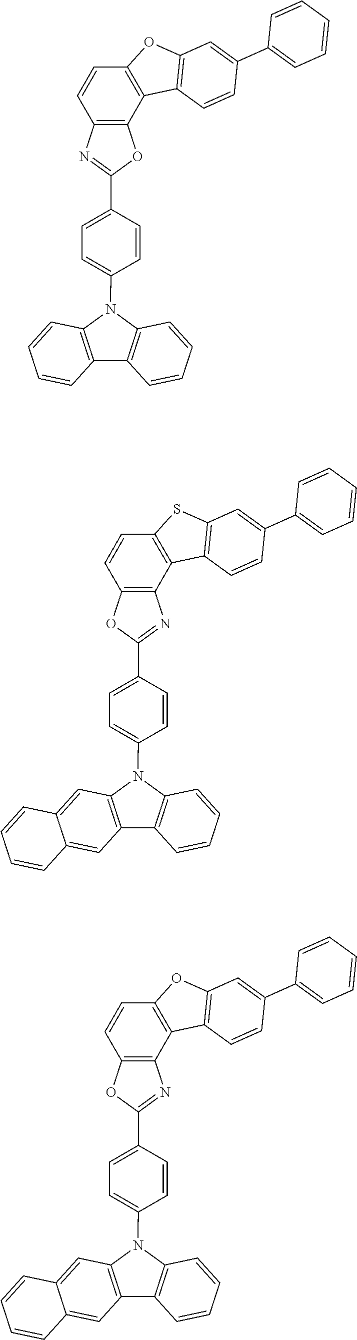 [SMILES: c1ccc(-c2ccc3c(c2)oc2ccc4nc(-c5ccc(-n6c7ccccc7c7ccccc76)cc5)oc4c23)cc1 ; c1ccc(-c2ccc3c(c2)sc2ccc4oc(-c5ccc(-n6c7ccccc7c7cc8ccccc8cc76)cc5)nc4c23)cc1 ; c1ccc(-c2ccc3c(c2)oc2ccc4oc(-c5ccc(-n6c7ccccc7c7cc8ccccc8cc76)cc5)nc4c23)cc1]

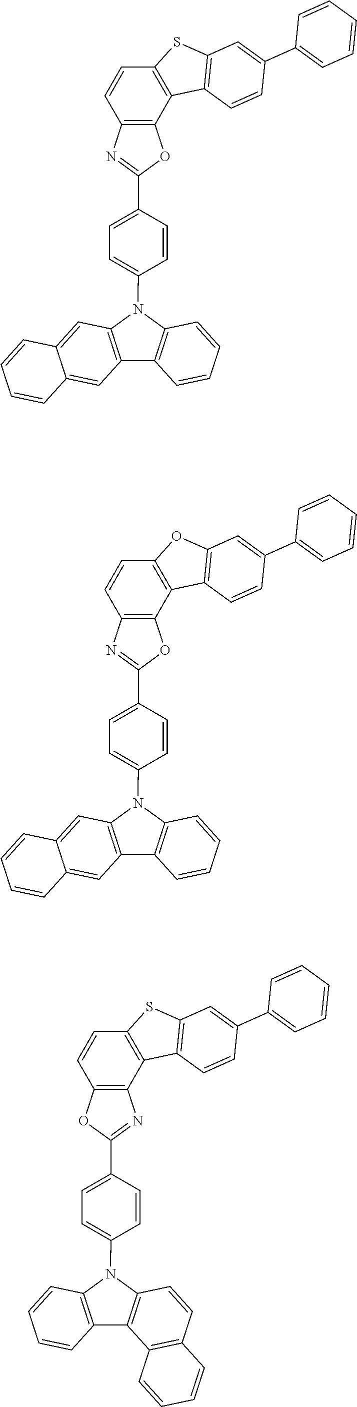 [SMILES: c1ccc(-c2ccc3c(c2)sc2ccc4nc(-c5ccc(-n6c7ccccc7c7cc8ccccc8cc76)cc5)oc4c23)cc1 ; c1ccc(-c2ccc3c(c2)oc2ccc4nc(-c5ccc(-n6c7ccccc7c7cc8ccccc8cc76)cc5)oc4c23)cc1 ; c1ccc(-c2ccc3c(c2)sc2ccc4oc(-c5ccc(-n6c7ccccc7c7c8ccccc8ccc76)cc5)nc4c23)cc1]

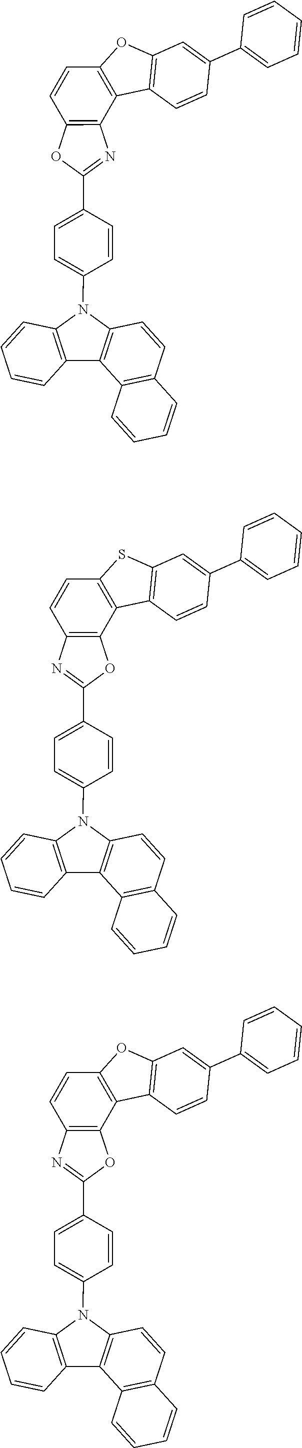 [SMILES: c1ccc(-c2ccc3c(c2)oc2ccc4oc(-c5ccc(-n6c7ccccc7c7c8ccccc8ccc76)cc5)nc4c23)cc1 ; c1ccc(-c2ccc3c(c2)sc2ccc4nc(-c5ccc(-n6c7ccccc7c7c8ccccc8ccc76)cc5)oc4c23)cc1 ; c1ccc(-c2ccc3c(c2)oc2ccc4nc(-c5ccc(-n6c7ccccc7c7c8ccccc8ccc76)cc5)oc4c23)cc1]

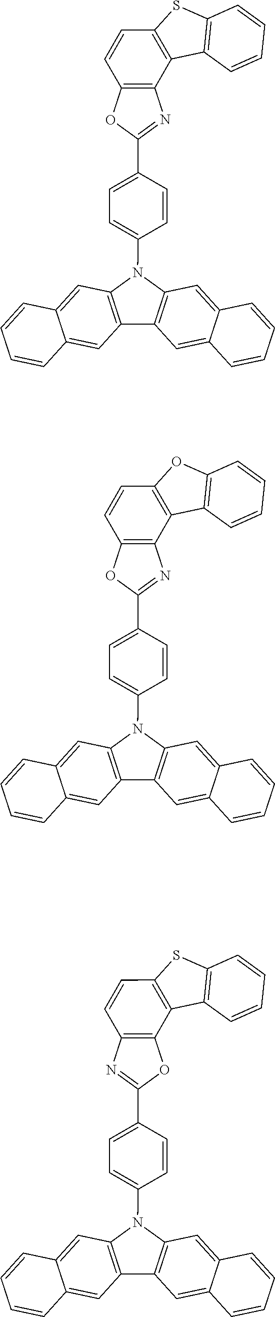 [SMILES: c1ccc2cc3c(cc2c1)c1cc2ccccc2cc1n3-c1ccc(-c2nc3c(ccc4sc5ccccc5c43)o2)cc1 ; c1ccc2cc3c(cc2c1)c1cc2ccccc2cc1n3-c1ccc(-c2nc3c(ccc4oc5ccccc5c43)o2)cc1 ; c1ccc2cc3c(cc2c1)c1cc2ccccc2cc1n3-c1ccc(-c2nc3ccc4sc5ccccc5c4c3o2)cc1]

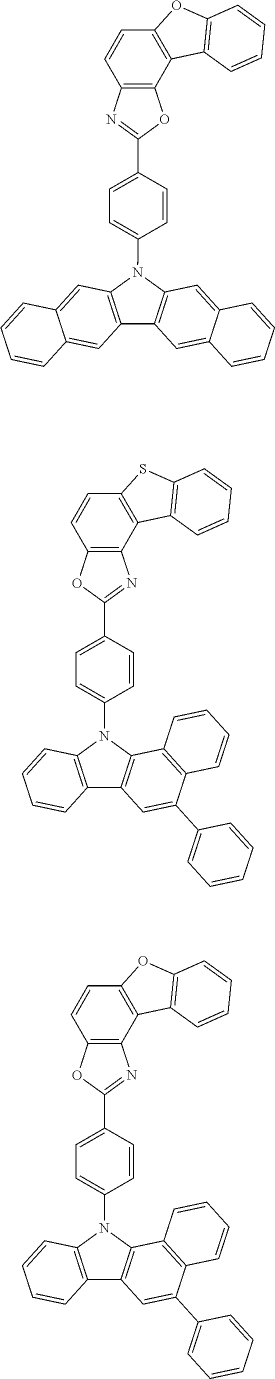 [SMILES: c1ccc2cc3c(cc2c1)c1cc2ccccc2cc1n3-c1ccc(-c2nc3ccc4oc5ccccc5c4c3o2)cc1 ; c1ccc(-c2cc3c4ccccc4n(-c4ccc(-c5nc6c(ccc7sc8ccccc8c76)o5)cc4)c3c3ccccc23)cc1 ; c1ccc(-c2cc3c4ccccc4n(-c4ccc(-c5nc6c(ccc7oc8ccccc8c76)o5)cc4)c3c3ccccc23)cc1]

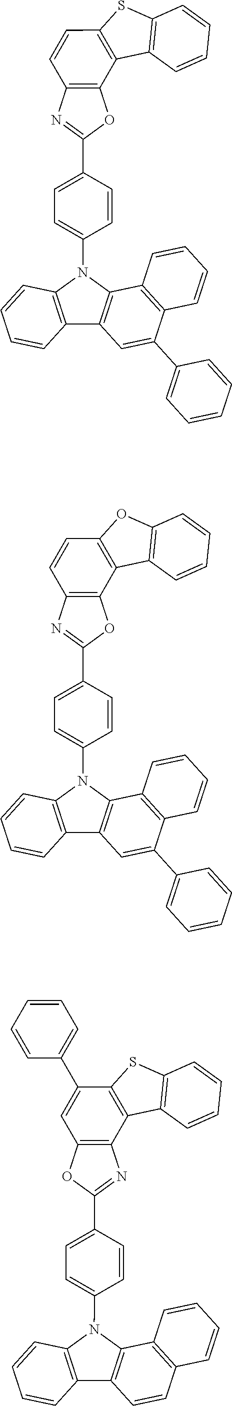 [SMILES: c1ccc(-c2cc3c4ccccc4n(-c4ccc(-c5nc6ccc7sc8ccccc8c7c6o5)cc4)c3c3ccccc23)cc1 ; c1ccc(-c2cc3c4ccccc4n(-c4ccc(-c5nc6ccc7oc8ccccc8c7c6o5)cc4)c3c3ccccc23)cc1 ; c1ccc(-c2cc3oc(-c4ccc(-n5c6ccccc6c6ccc7ccccc7c65)cc4)nc3c3c2sc2ccccc23)cc1]

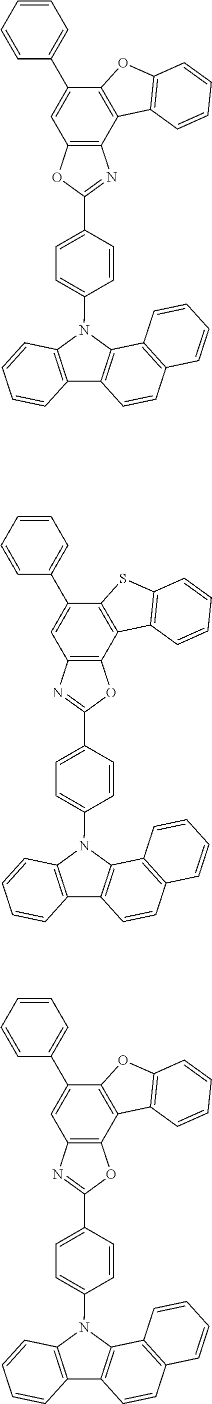 [SMILES: c1ccc(-c2cc3oc(-c4ccc(-n5c6ccccc6c6ccc7ccccc7c65)cc4)nc3c3c2oc2ccccc23)cc1 ; c1ccc(-c2cc3nc(-c4ccc(-n5c6ccccc6c6ccc7ccccc7c65)cc4)oc3c3c2sc2ccccc23)cc1 ; c1ccc(-c2cc3nc(-c4ccc(-n5c6ccccc6c6ccc7ccccc7c65)cc4)oc3c3c2oc2ccccc23)cc1]

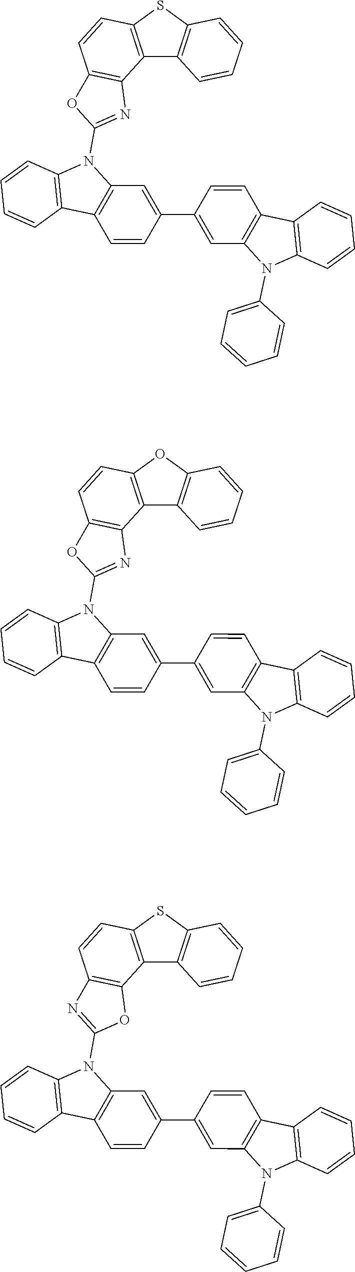 [SMILES: c1ccc(-n2c3ccccc3c3ccc(-c4ccc5c6ccccc6n(-c6nc7c(ccc8sc9ccccc9c87)o6)c5c4)cc32)cc1 ; c1ccc(-n2c3ccccc3c3ccc(-c4ccc5c6ccccc6n(-c6nc7c(ccc8oc9ccccc9c87)o6)c5c4)cc32)cc1 ; c1ccc(-n2c3ccccc3c3ccc(-c4ccc5c6ccccc6n(-c6nc7ccc8sc9ccccc9c8c7o6)c5c4)cc32)cc1]

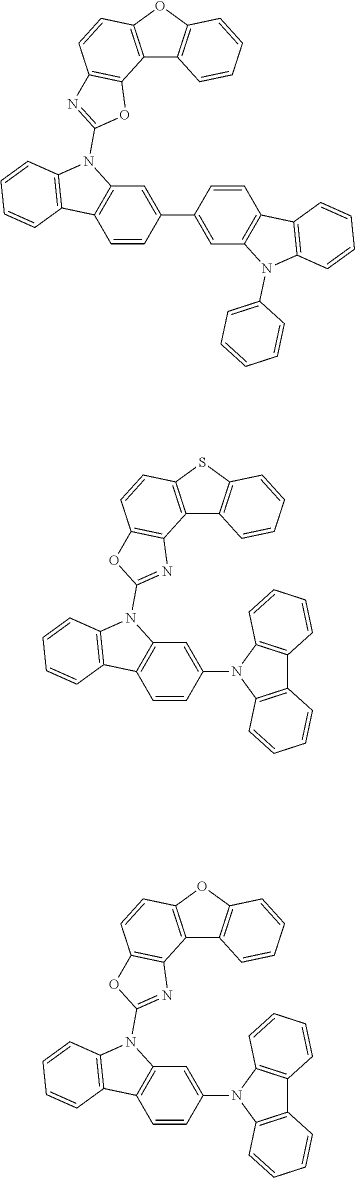 [SMILES: c1ccc(-n2c3ccccc3c3ccc(-c4ccc5c6ccccc6n(-c6nc7ccc8oc9ccccc9c8c7o6)c5c4)cc32)cc1 ; c1ccc2c(c1)sc1ccc3oc(-n4c5ccccc5c5ccc(-n6c7ccccc7c7ccccc76)cc54)nc3c12 ; c1ccc2c(c1)oc1ccc3oc(-n4c5ccccc5c5ccc(-n6c7ccccc7c7ccccc76)cc54)nc3c12]

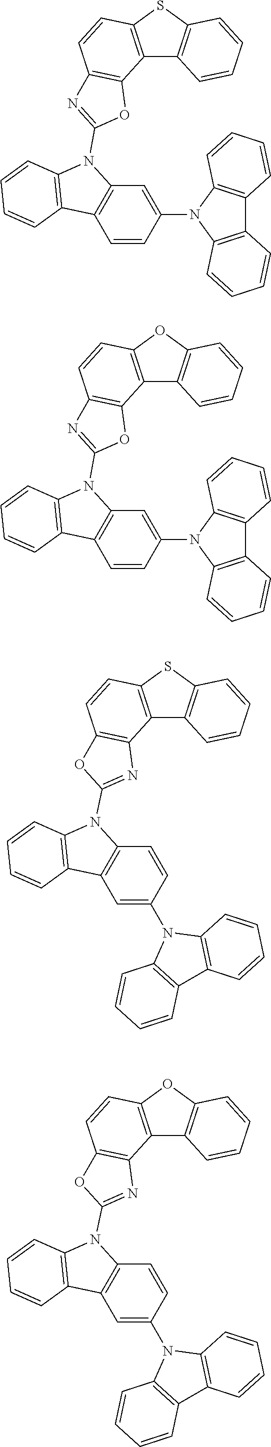 [SMILES: c1ccc2c(c1)sc1ccc3nc(-n4c5ccccc5c5ccc(-n6c7ccccc7c7ccccc76)cc54)oc3c12 ; c1ccc2c(c1)oc1ccc3nc(-n4c5ccccc5c5ccc(-n6c7ccccc7c7ccccc76)cc54)oc3c12 ; c1ccc2c(c1)sc1ccc3oc(-n4c5ccccc5c5cc(-n6c7ccccc7c7ccccc76)ccc54)nc3c12 ; c1ccc2c(c1)oc1ccc3oc(-n4c5ccccc5c5cc(-n6c7ccccc7c7ccccc76)ccc54)nc3c12]

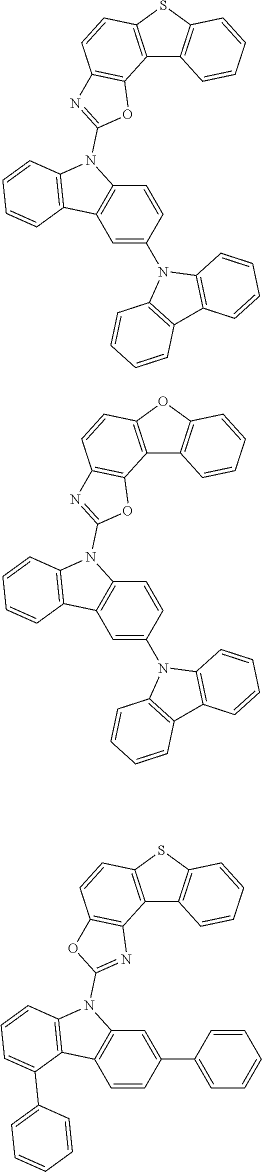

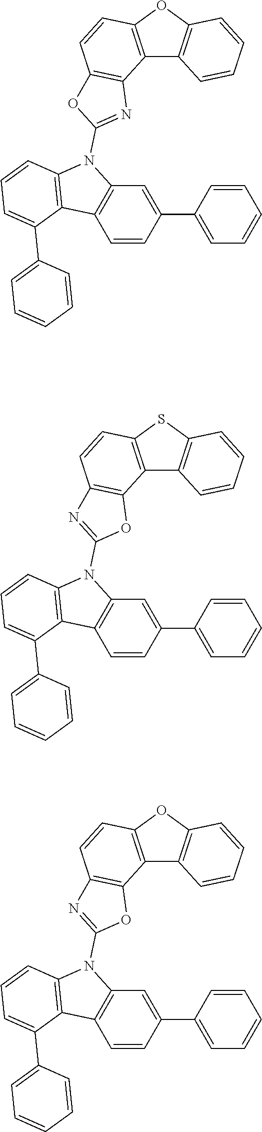 [SMILES: c1ccc(-c2ccc3c4c(-c5ccccc5)cccc4n(-c4nc5c(ccc6oc7ccccc7c65)o4)c3c2)cc1 ; c1ccc(-c2ccc3c4c(-c5ccccc5)cccc4n(-c4nc5ccc6sc7ccccc7c6c5o4)c3c2)cc1 ; c1ccc(-c2ccc3c4c(-c5ccccc5)cccc4n(-c4nc5ccc6oc7ccccc7c6c5o4)c3c2)cc1]

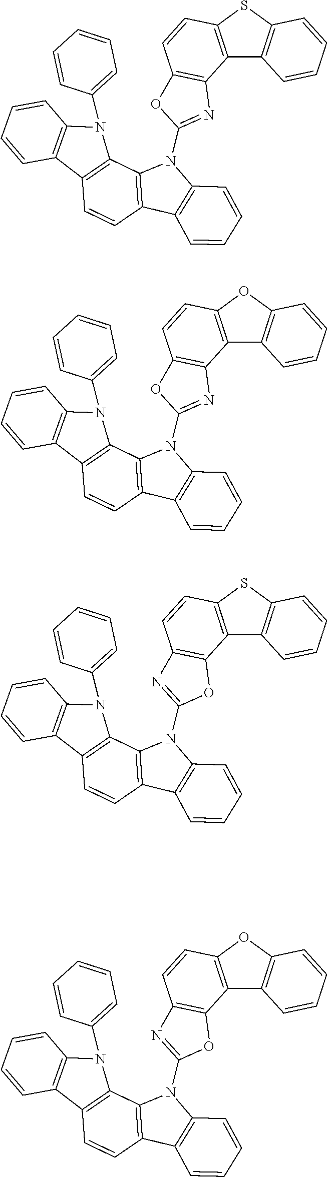 [SMILES: c1ccc(-n2c3ccccc3c3ccc4c5ccccc5n(-c5nc6c(ccc7sc8ccccc8c76)o5)c4c32)cc1 ; c1ccc(-n2c3ccccc3c3ccc4c5ccccc5n(-c5nc6c(ccc7oc8ccccc8c76)o5)c4c32)cc1 ; c1ccc(-n2c3ccccc3c3ccc4c5ccccc5n(-c5nc6ccc7sc8ccccc8c7c6o5)c4c32)cc1 ; c1ccc(-n2c3ccccc3c3ccc4c5ccccc5n(-c5nc6ccc7oc8ccccc8c7c6o5)c4c32)cc1]

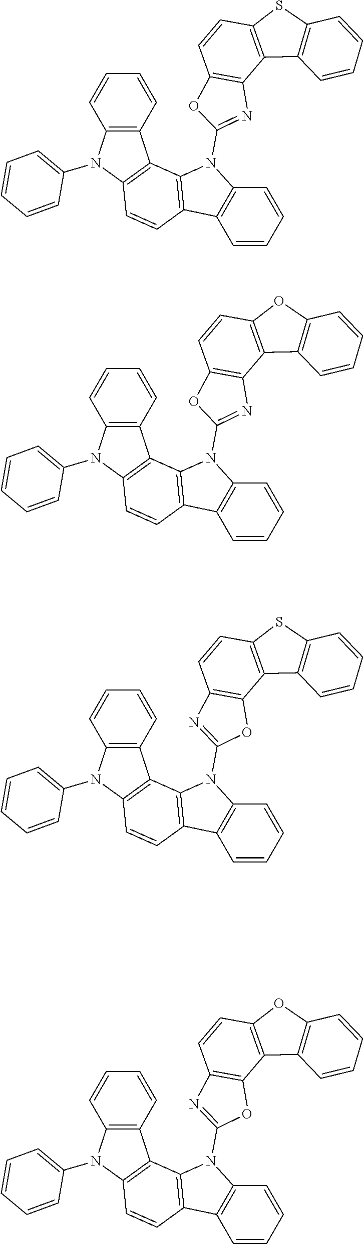 [SMILES: c1ccc(-n2c3ccccc3c3c2ccc2c4ccccc4n(-c4nc5c(ccc6sc7ccccc7c65)o4)c23)cc1 ; c1ccc(-n2c3ccccc3c3c2ccc2c4ccccc4n(-c4nc5c(ccc6oc7ccccc7c65)o4)c23)cc1 ; c1ccc(-n2c3ccccc3c3c2ccc2c4ccccc4n(-c4nc5ccc6sc7ccccc7c6c5o4)c23)cc1 ; c1ccc(-n2c3ccccc3c3c2ccc2c4ccccc4n(-c4nc5ccc6oc7ccccc7c6c5o4)c23)cc1]

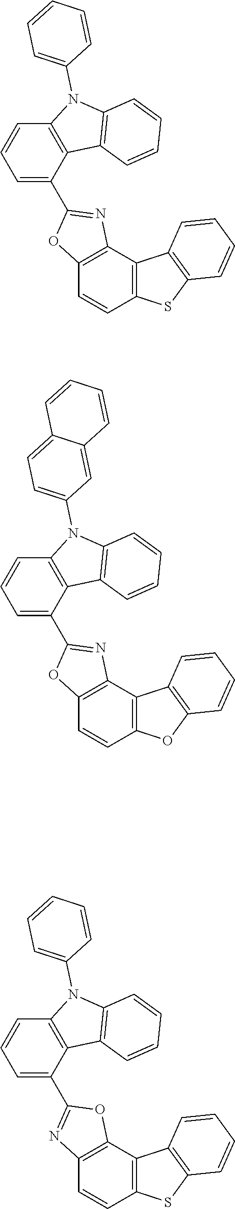 [SMILES: c1ccc(-n2c3ccccc3c3c(-c4nc5c(ccc6sc7ccccc7c65)o4)cccc32)cc1 ; c1ccc2cc(-n3c4ccccc4c4c(-c5nc6c(ccc7oc8ccccc8c76)o5)cccc43)ccc2c1 ; c1ccc(-n2c3ccccc3c3c(-c4nc5ccc6sc7ccccc7c6c5o4)cccc32)cc1]

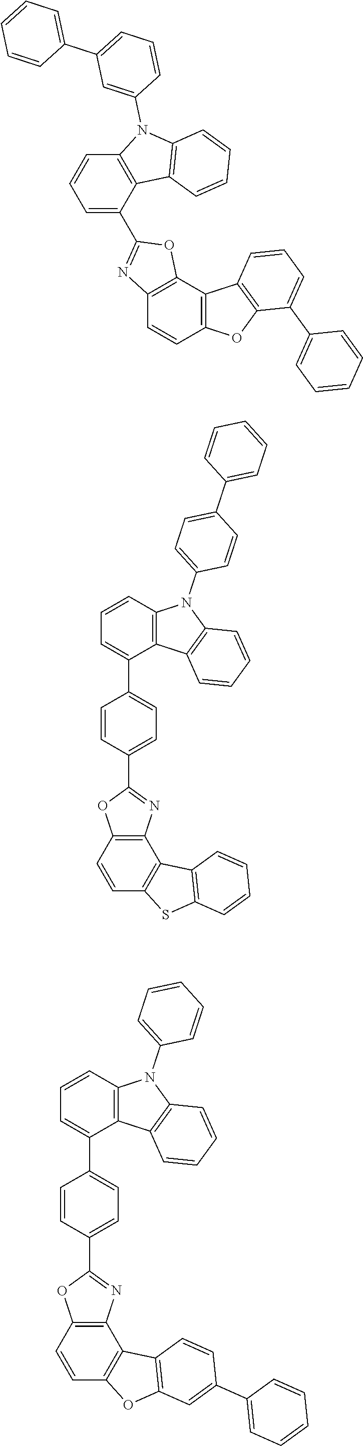 [SMILES: c1ccc(-c2cccc(-n3c4ccccc4c4c(-c5nc6ccc7oc8c(-c9ccccc9)cccc8c7c6o5)cccc43)c2)cc1 ; c1ccc(-c2ccc(-n3c4ccccc4c4c(-c5ccc(-c6nc7c(ccc8sc9ccccc9c87)o6)cc5)cccc43)cc2)cc1 ; c1ccc(-c2ccc3c(c2)oc2ccc4oc(-c5ccc(-c6cccc7c6c6ccccc6n7-c6ccccc6)cc5)nc4c23)cc1]

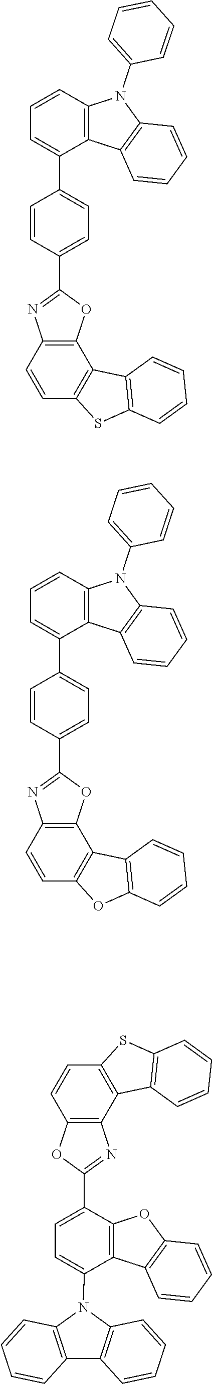 [SMILES: c1ccc(-n2c3ccccc3c3c(-c4ccc(-c5nc6ccc7sc8ccccc8c7c6o5)cc4)cccc32)cc1 ; c1ccc(-n2c3ccccc3c3c(-c4ccc(-c5nc6ccc7oc8ccccc8c7c6o5)cc4)cccc32)cc1 ; c1ccc2c(c1)oc1c(-c3nc4c(ccc5sc6ccccc6c54)o3)ccc(-n3c4ccccc4c4ccccc43)c12]

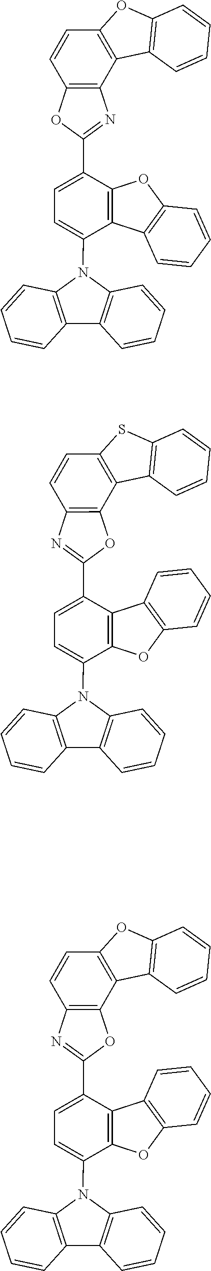 [SMILES: c1ccc2c(c1)oc1c(-c3nc4c(ccc5oc6ccccc6c54)o3)ccc(-n3c4ccccc4c4ccccc43)c12 ; c1ccc2c(c1)oc1c(-n3c4ccccc4c4ccccc43)ccc(-c3nc4ccc5sc6ccccc6c5c4o3)c12 ; c1ccc2c(c1)oc1c(-n3c4ccccc4c4ccccc43)ccc(-c3nc4ccc5oc6ccccc6c5c4o3)c12]

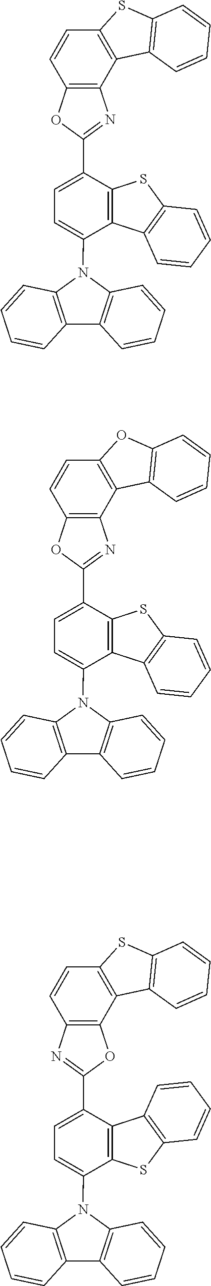 [SMILES: c1ccc2c(c1)sc1c(-c3nc4c(ccc5sc6ccccc6c54)o3)ccc(-n3c4ccccc4c4ccccc43)c12 ; c1ccc2c(c1)oc1ccc3oc(-c4ccc(-n5c6ccccc6c6ccccc65)c5c4sc4ccccc45)nc3c12 ; c1ccc2c(c1)sc1c(-n3c4ccccc4c4ccccc43)ccc(-c3nc4ccc5sc6ccccc6c5c4o3)c12]

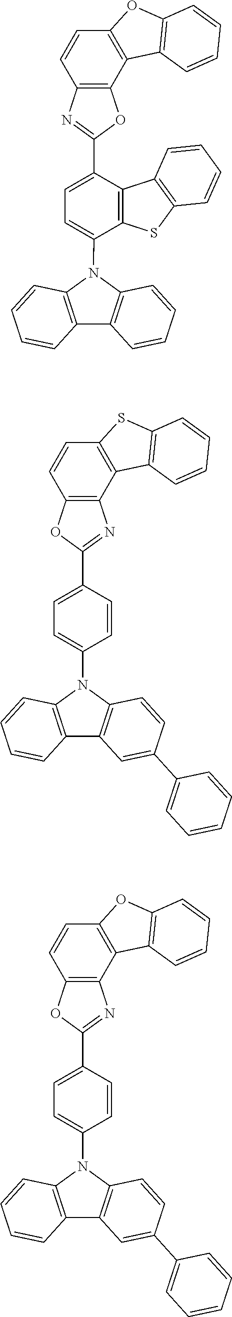 [SMILES: c1ccc2c(c1)oc1ccc3nc(-c4ccc(-n5c6ccccc6c6ccccc65)c5sc6ccccc6c45)oc3c12 ; c1ccc(-c2ccc3c(c2)c2ccccc2n3-c2ccc(-c3nc4c(ccc5sc6ccccc6c54)o3)cc2)cc1 ; c1ccc(-c2ccc3c(c2)c2ccccc2n3-c2ccc(-c3nc4c(ccc5oc6ccccc6c54)o3)cc2)cc1]

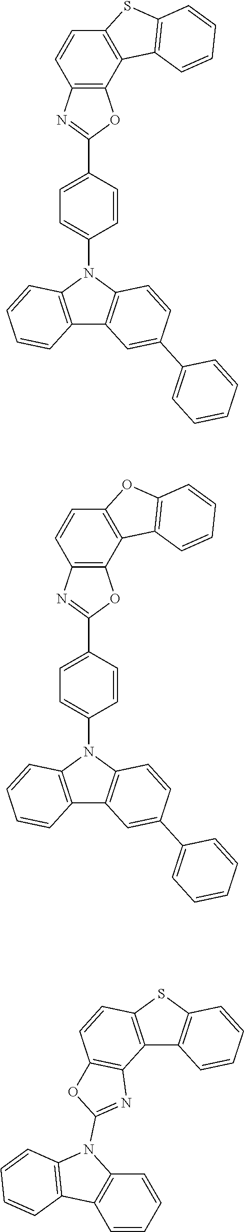 [SMILES: c1ccc(-c2ccc3c(c2)c2ccccc2n3-c2ccc(-c3nc4ccc5sc6ccccc6c5c4o3)cc2)cc1 ; c1ccc(-c2ccc3c(c2)c2ccccc2n3-c2ccc(-c3nc4ccc5oc6ccccc6c5c4o3)cc2)cc1 ; c1ccc2c(c1)sc1ccc3oc(-n4c5ccccc5c5ccccc54)nc3c12]

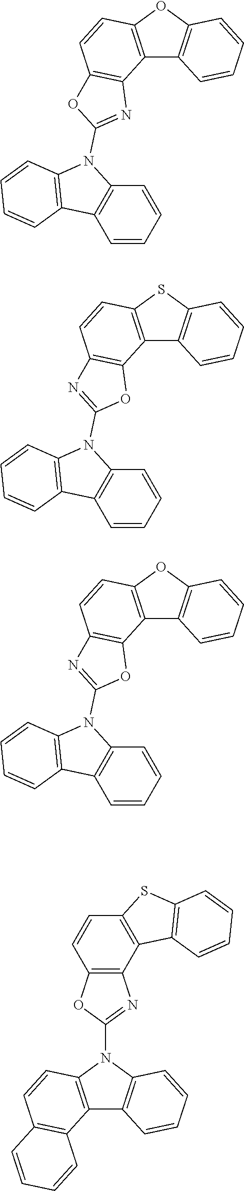

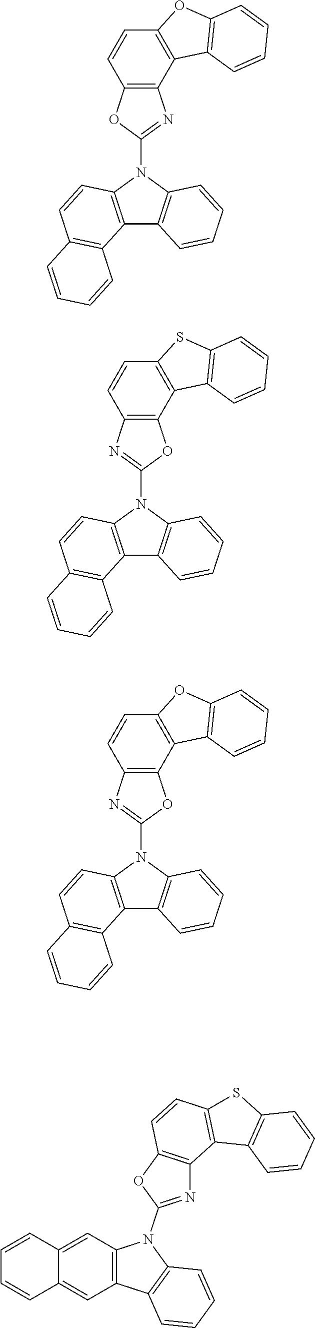 [SMILES: c1ccc2c(c1)ccc1c2c2ccccc2n1-c1nc2c(ccc3oc4ccccc4c32)o1 ; c1ccc2c(c1)ccc1c2c2ccccc2n1-c1nc2ccc3sc4ccccc4c3c2o1 ; c1ccc2c(c1)ccc1c2c2ccccc2n1-c1nc2ccc3oc4ccccc4c3c2o1 ; c1ccc2cc3c(cc2c1)c1ccccc1n3-c1nc2c(ccc3sc4ccccc4c32)o1]

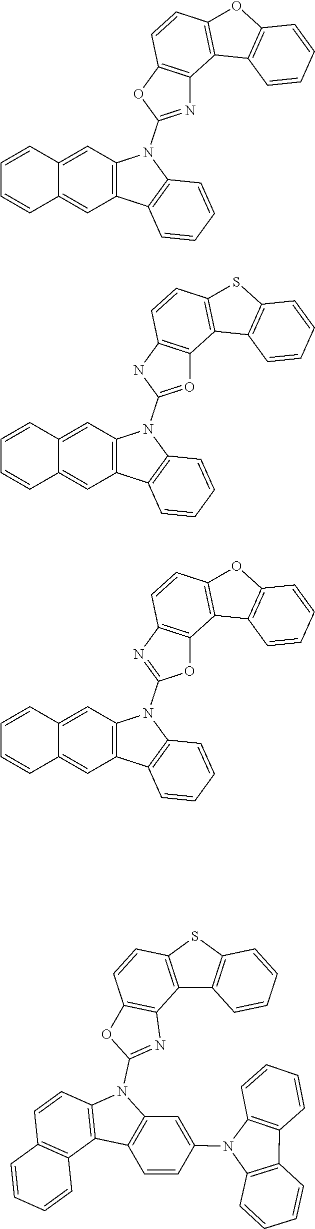

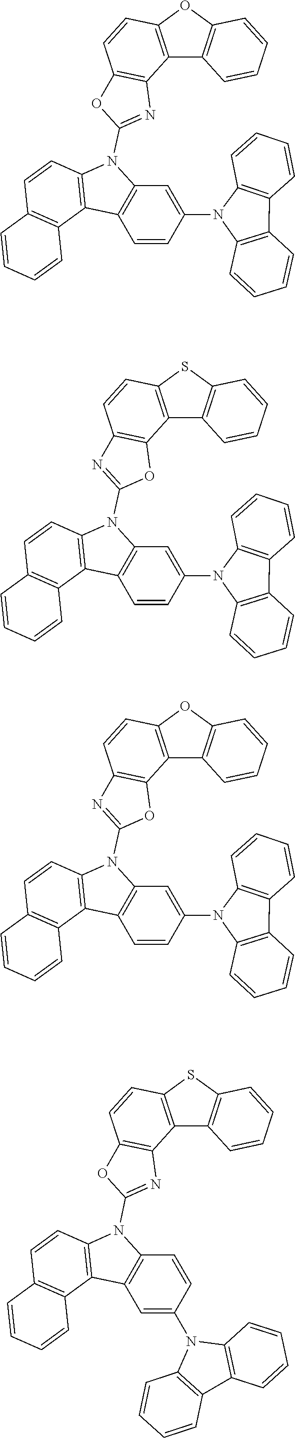 [SMILES: c1ccc2c(c1)ccc1c2c2ccc(-n3c4ccccc4c4ccccc43)cc2n1-c1nc2c(ccc3oc4ccccc4c32)o1 ; c1ccc2c(c1)ccc1c2c2ccc(-n3c4ccccc4c4ccccc43)cc2n1-c1nc2ccc3sc4ccccc4c3c2o1 ; c1ccc2c(c1)ccc1c2c2ccc(-n3c4ccccc4c4ccccc43)cc2n1-c1nc2ccc3oc4ccccc4c3c2o1 ; c1ccc2c(c1)ccc1c2c2cc(-n3c4ccccc4c4ccccc43)ccc2n1-c1nc2c(ccc3sc4ccccc4c32)o1]

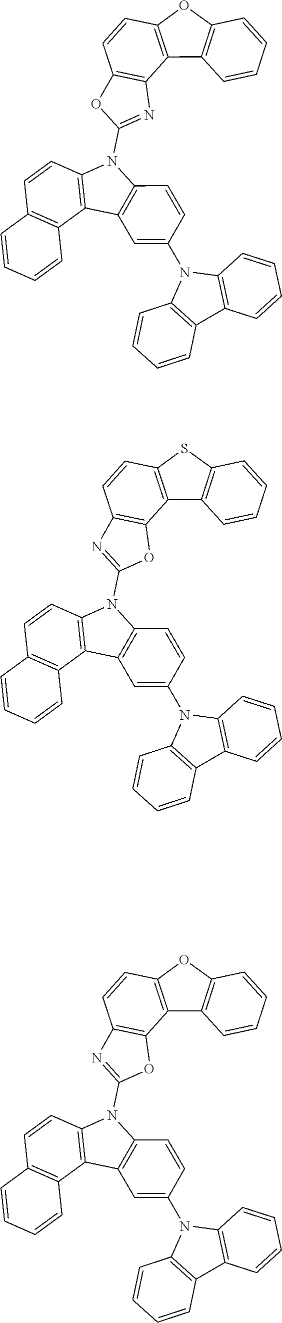 [SMILES: c1ccc2c(c1)ccc1c2c2cc(-n3c4ccccc4c4ccccc43)ccc2n1-c1nc2c(ccc3oc4ccccc4c32)o1 ; c1ccc2c(c1)ccc1c2c2cc(-n3c4ccccc4c4ccccc43)ccc2n1-c1nc2ccc3sc4ccccc4c3c2o1 ; c1ccc2c(c1)ccc1c2c2cc(-n3c4ccccc4c4ccccc43)ccc2n1-c1nc2ccc3oc4ccccc4c3c2o1]

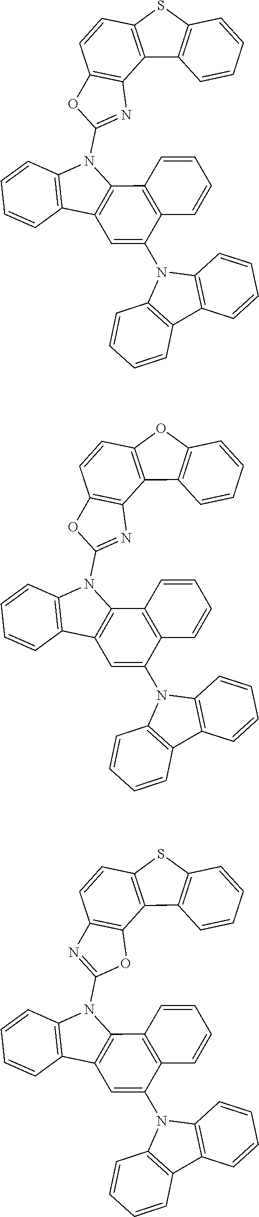 [SMILES: c1ccc2c(c1)sc1ccc3oc(-n4c5ccccc5c5cc(-n6c7ccccc7c7ccccc76)c6ccccc6c54)nc3c12 ; c1ccc2c(c1)oc1ccc3oc(-n4c5ccccc5c5cc(-n6c7ccccc7c7ccccc76)c6ccccc6c54)nc3c12 ; c1ccc2c(c1)sc1ccc3nc(-n4c5ccccc5c5cc(-n6c7ccccc7c7ccccc76)c6ccccc6c54)oc3c12]

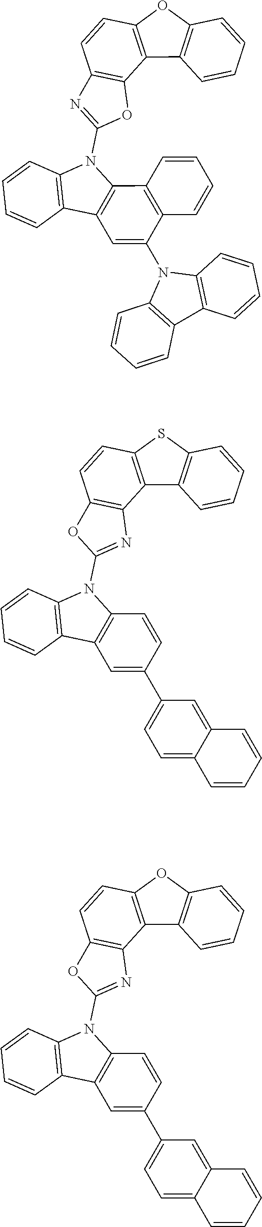 [SMILES: c1ccc2c(c1)oc1ccc3nc(-n4c5ccccc5c5cc(-n6c7ccccc7c7ccccc76)c6ccccc6c54)oc3c12 ; c1ccc2cc(-c3ccc4c(c3)c3ccccc3n4-c3nc4c(ccc5sc6ccccc6c54)o3)ccc2c1 ; c1ccc2cc(-c3ccc4c(c3)c3ccccc3n4-c3nc4c(ccc5oc6ccccc6c54)o3)ccc2c1]

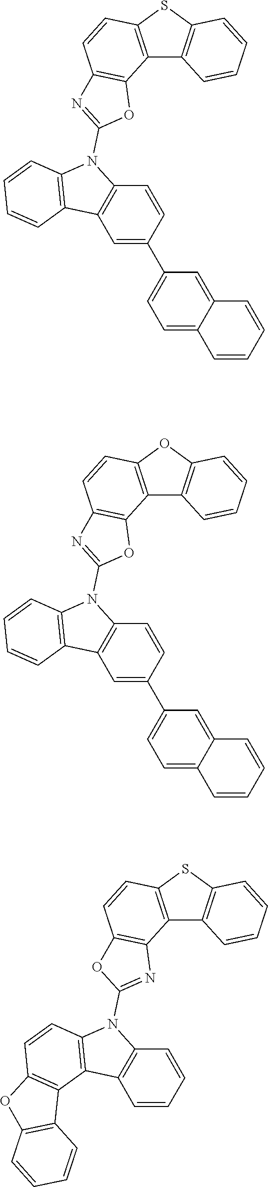 [SMILES: c1ccc2cc(-c3ccc4c(c3)c3ccccc3n4-c3nc4ccc5sc6ccccc6c5c4o3)ccc2c1 ; c1ccc2cc(-c3ccc4c(c3)c3ccccc3n4-c3nc4ccc5oc6ccccc6c5c4o3)ccc2c1 ; c1ccc2c(c1)oc1ccc3c(c4ccccc4n3-c3nc4c(ccc5sc6ccccc6c54)o3)c12]

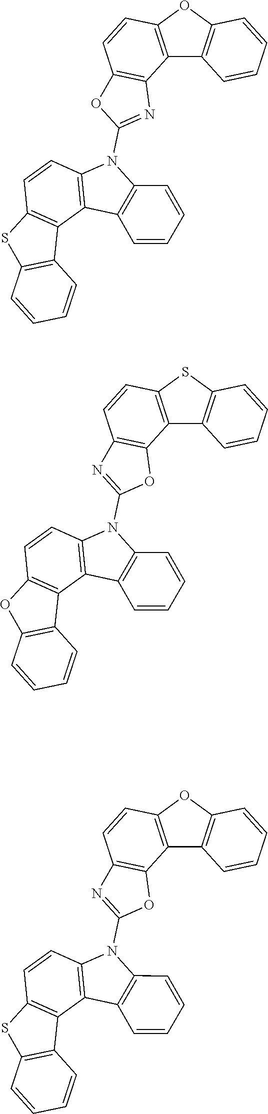 [SMILES: c1ccc2c(c1)oc1ccc3oc(-n4c5ccccc5c5c6c(ccc54)sc4ccccc46)nc3c12 ; c1ccc2c(c1)oc1ccc3c(c4ccccc4n3-c3nc4ccc5sc6ccccc6c5c4o3)c12 ; c1ccc2c(c1)oc1ccc3nc(-n4c5ccccc5c5c6c(ccc54)sc4ccccc46)oc3c12]

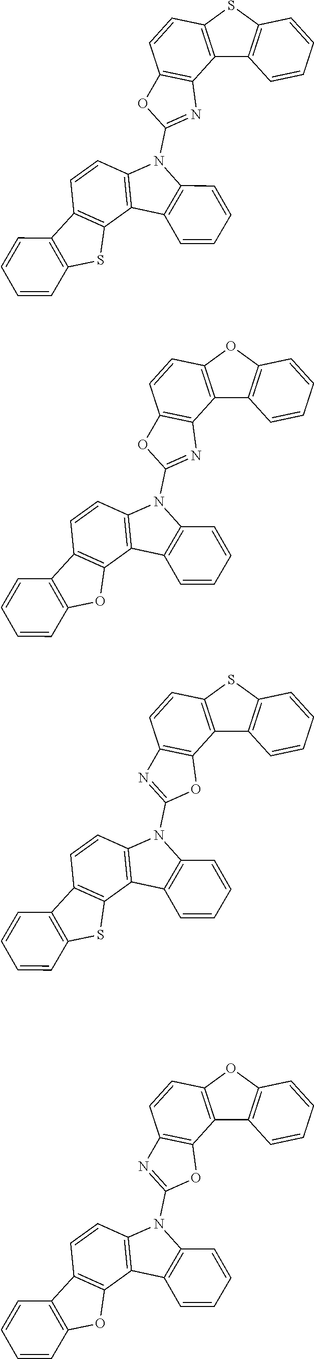 [SMILES: c1ccc2c(c1)sc1c2ccc2c1c1ccccc1n2-c1nc2c(ccc3sc4ccccc4c32)o1 ; c1ccc2c(c1)oc1c2ccc2c1c1ccccc1n2-c1nc2c(ccc3oc4ccccc4c32)o1 ; c1ccc2c(c1)sc1c2ccc2c1c1ccccc1n2-c1nc2ccc3sc4ccccc4c3c2o1 ; c1ccc2c(c1)oc1c2ccc2c1c1ccccc1n2-c1nc2ccc3oc4ccccc4c3c2o1]

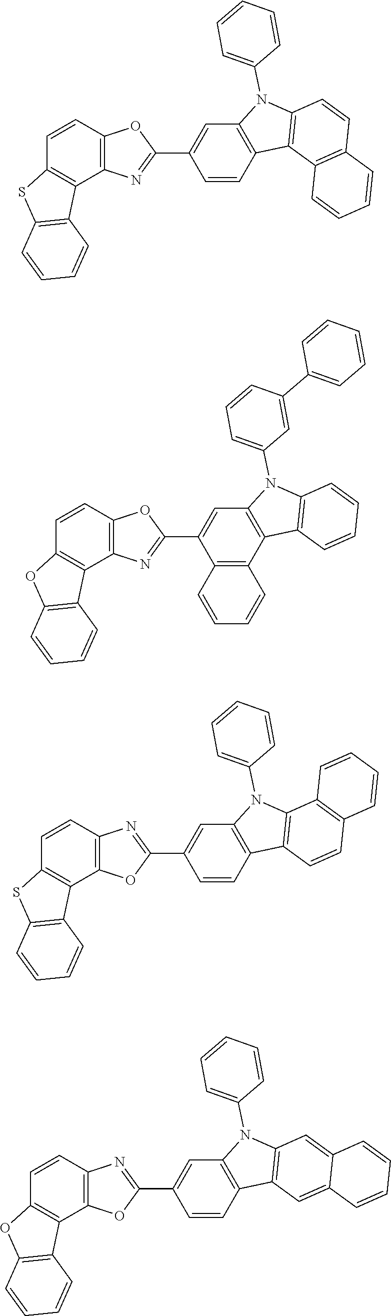 [SMILES: c1ccc(-n2c3cc(-c4nc5c(ccc6sc7ccccc7c65)o4)ccc3c3c4ccccc4ccc32)cc1 ; c1ccc(-c2cccc(-n3c4ccccc4c4c5ccccc5c(-c5nc6c(ccc7oc8ccccc8c76)o5)cc43)c2)cc1 ; c1ccc(-n2c3cc(-c4nc5ccc6sc7ccccc7c6c5o4)ccc3c3ccc4ccccc4c32)cc1 ; c1ccc(-n2c3cc(-c4nc5ccc6oc7ccccc7c6c5o4)ccc3c3cc4ccccc4cc32)cc1]

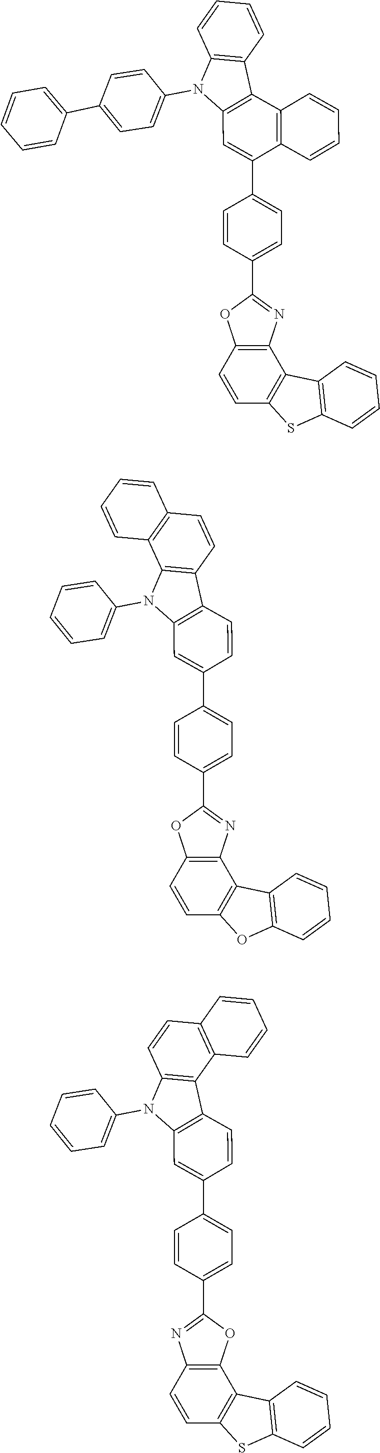 [SMILES: c1ccc(-c2ccc(-n3c4ccccc4c4c5ccccc5c(-c5ccc(-c6nc7c(ccc8sc9ccccc9c87)o6)cc5)cc43)cc2)cc1 ; c1ccc(-n2c3cc(-c4ccc(-c5nc6c(ccc7oc8ccccc8c76)o5)cc4)ccc3c3ccc4ccccc4c32)cc1 ; c1ccc(-n2c3cc(-c4ccc(-c5nc6ccc7sc8ccccc8c7c6o5)cc4)ccc3c3c4ccccc4ccc32)cc1]

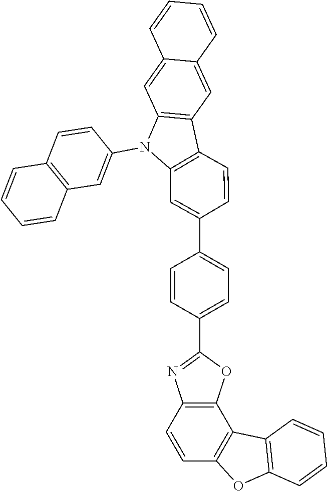 [SMILES: c1ccc2cc(-n3c4cc(-c5ccc(-c6nc7ccc8oc9ccccc9c8c7o6)cc5)ccc4c4cc5ccccc5cc43)ccc2c1]

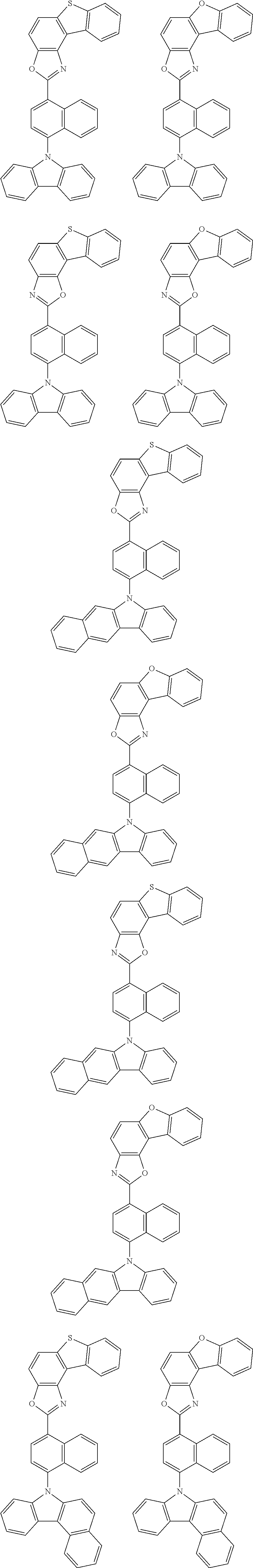

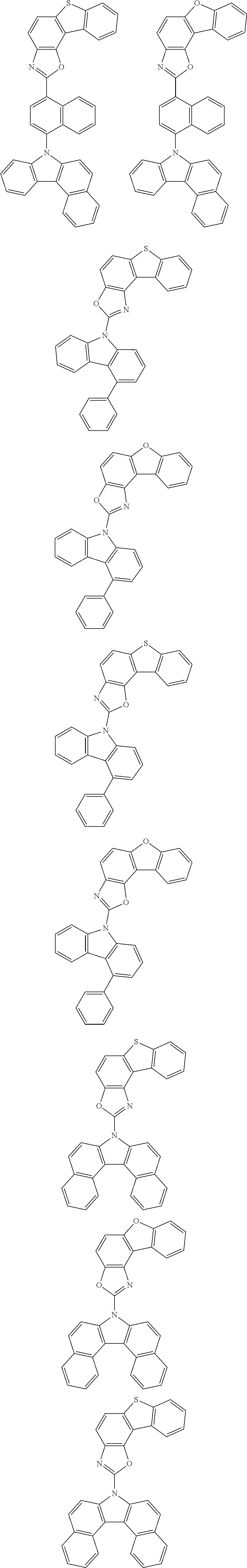

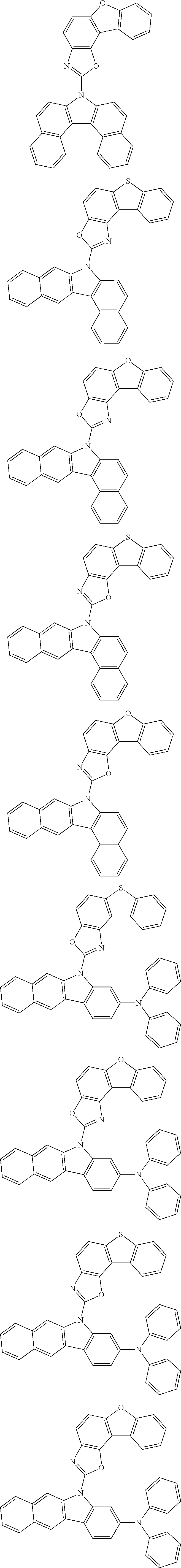

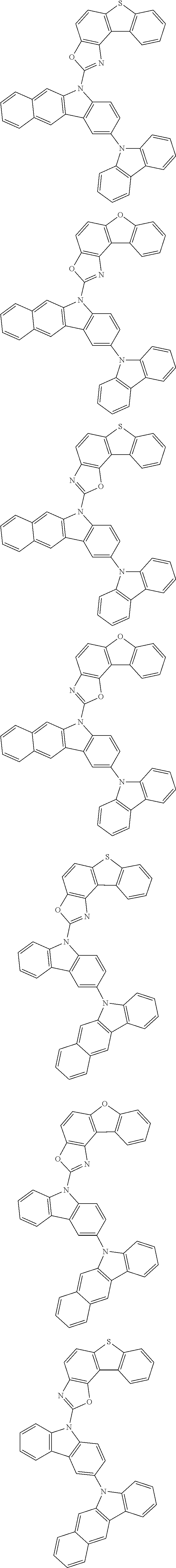

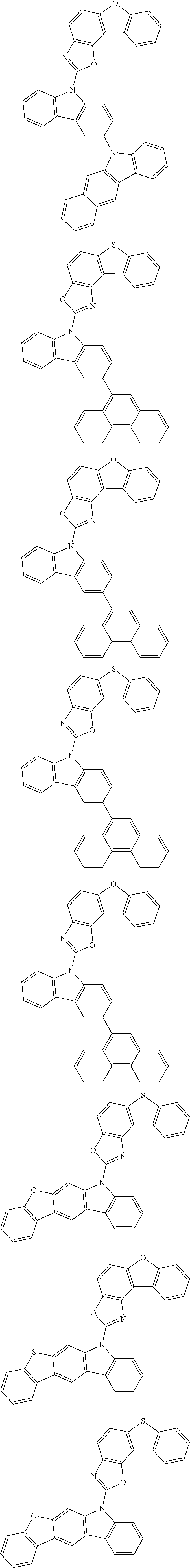

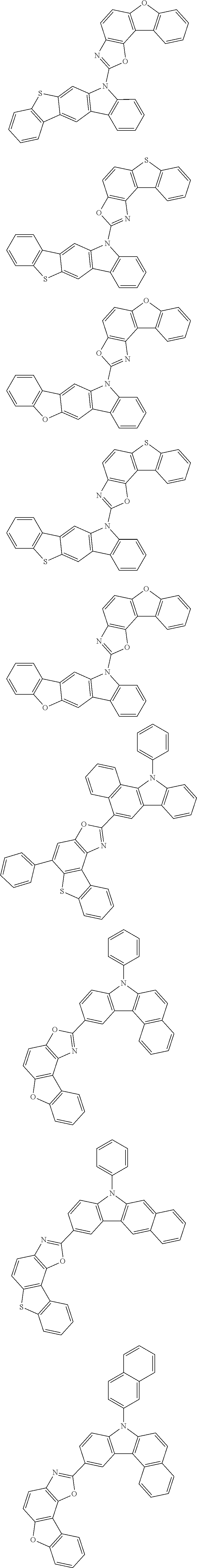

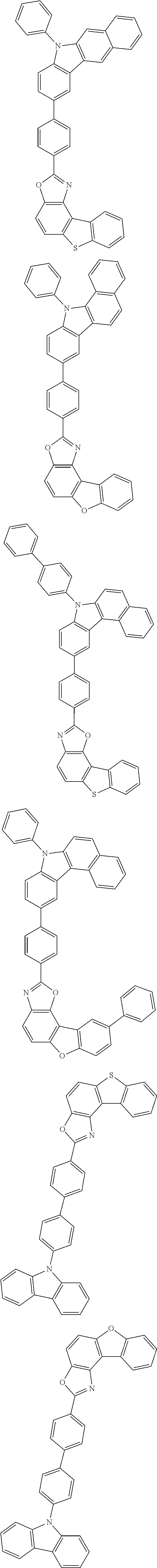

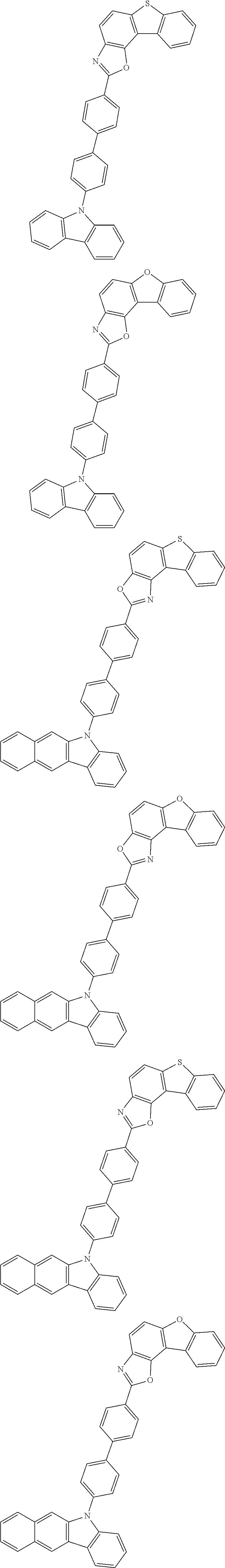

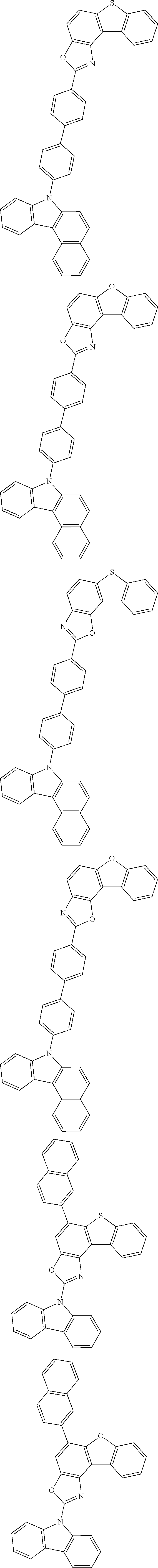

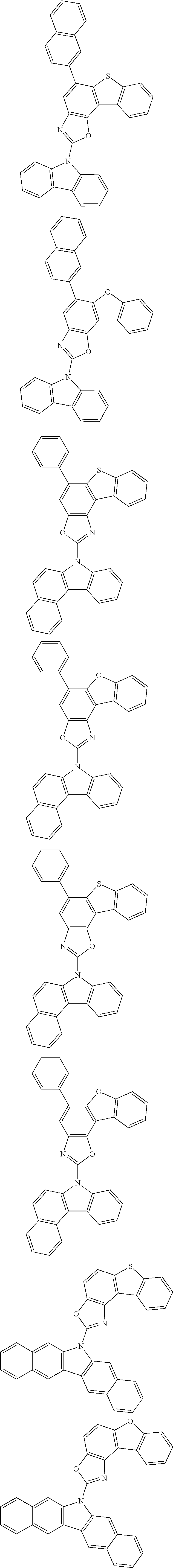

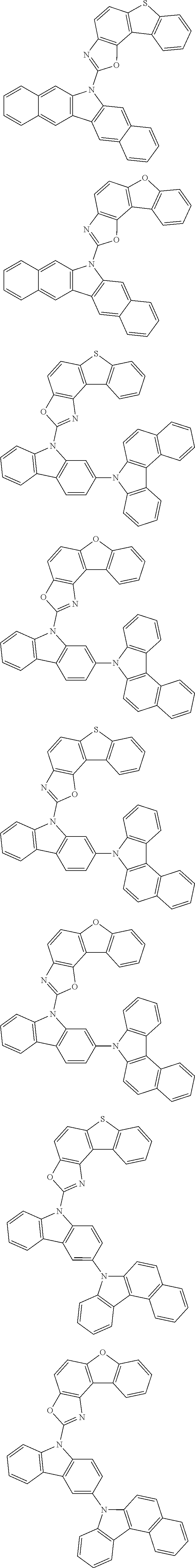

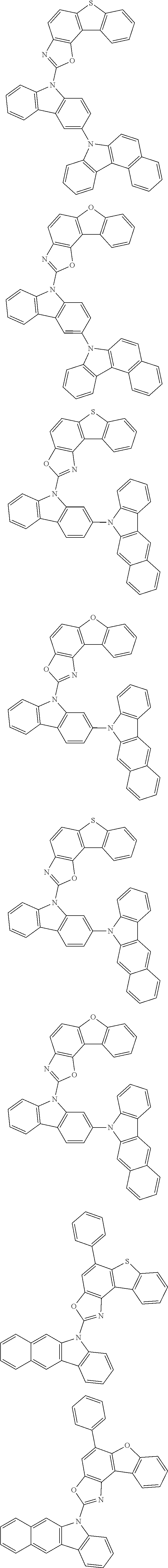

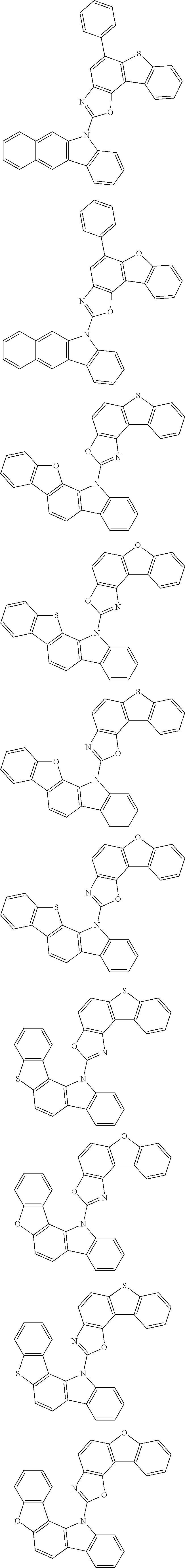

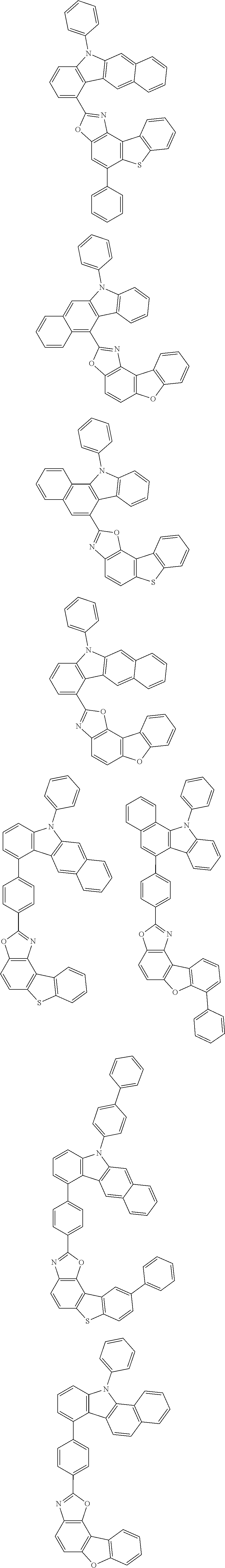

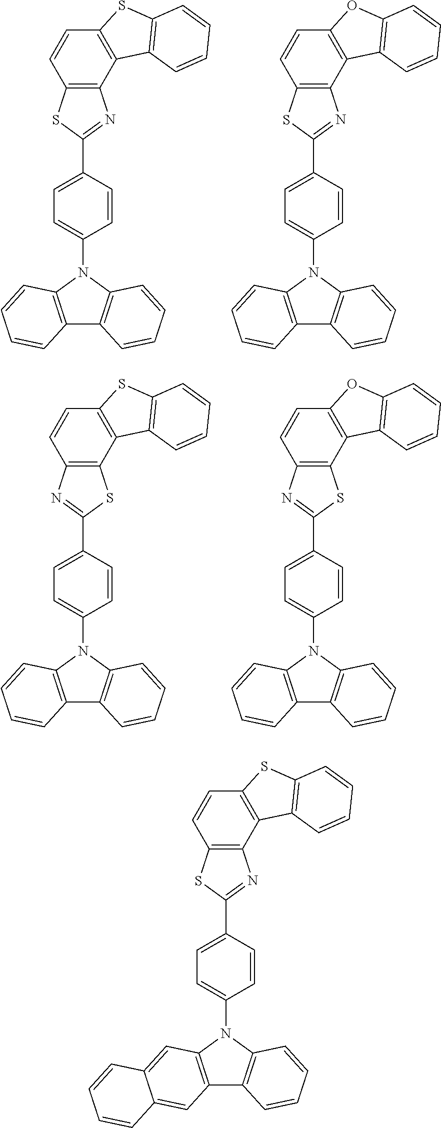

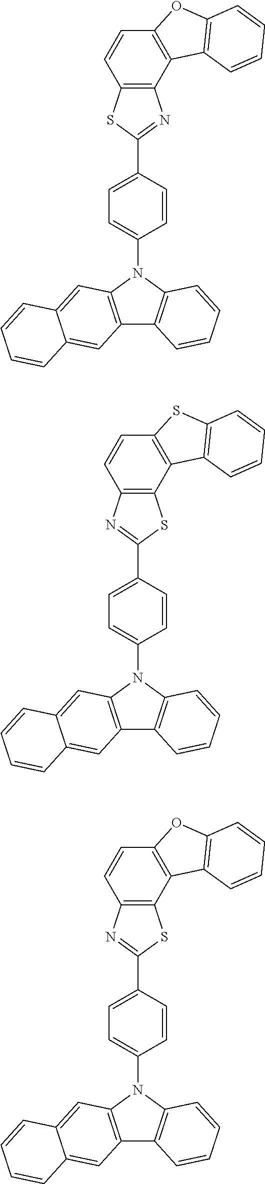 [SMILES: c1ccc2cc3c(cc2c1)c1ccccc1n3-c1ccc(-c2nc3c(ccc4oc5ccccc5c43)s2)cc1 ; c1ccc2cc3c(cc2c1)c1ccccc1n3-c1ccc(-c2nc3ccc4sc5ccccc5c4c3s2)cc1 ; c1ccc2cc3c(cc2c1)c1ccccc1n3-c1ccc(-c2nc3ccc4oc5ccccc5c4c3s2)cc1]

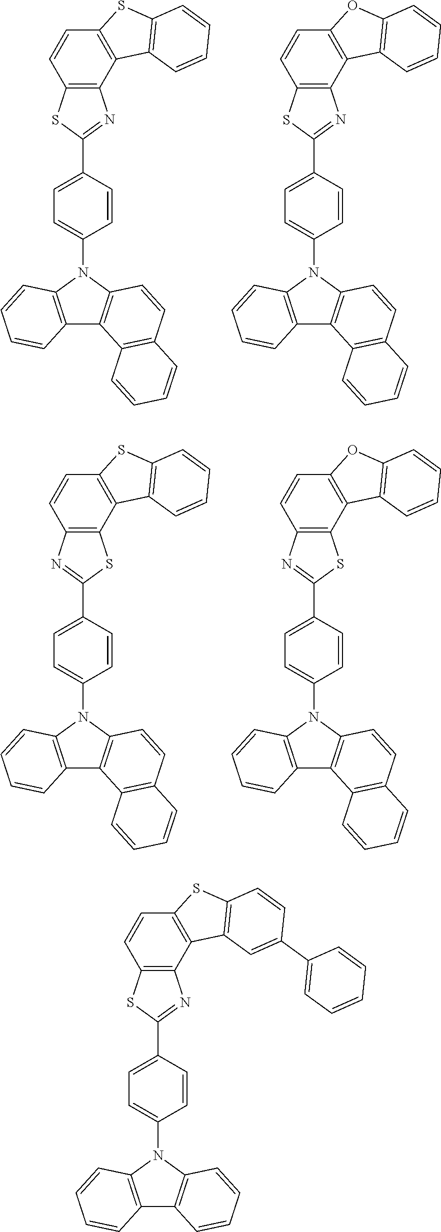

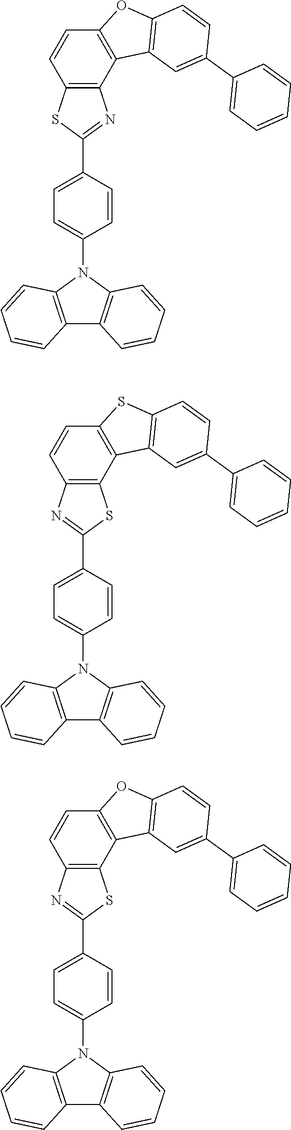

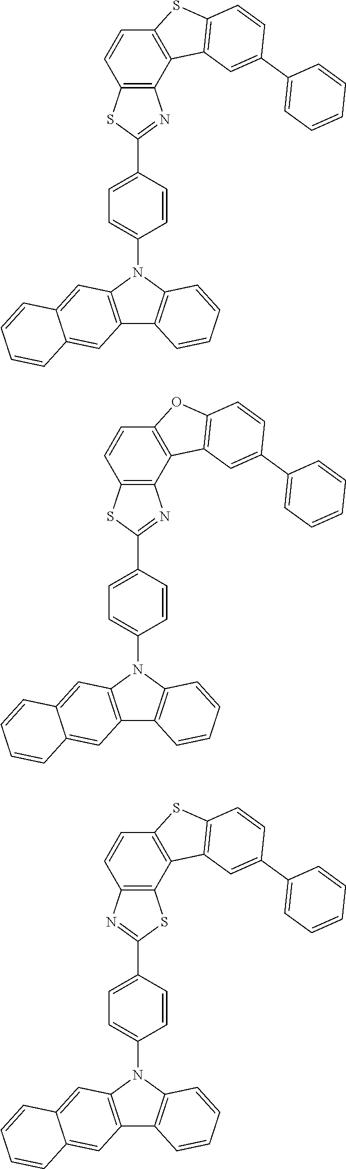 [SMILES: c1ccc(-c2ccc3sc4ccc5sc(-c6ccc(-n7c8ccccc8c8cc9ccccc9cc87)cc6)nc5c4c3c2)cc1 ; c1ccc(-c2ccc3oc4ccc5sc(-c6ccc(-n7c8ccccc8c8cc9ccccc9cc87)cc6)nc5c4c3c2)cc1 ; c1ccc(-c2ccc3sc4ccc5nc(-c6ccc(-n7c8ccccc8c8cc9ccccc9cc87)cc6)sc5c4c3c2)cc1]

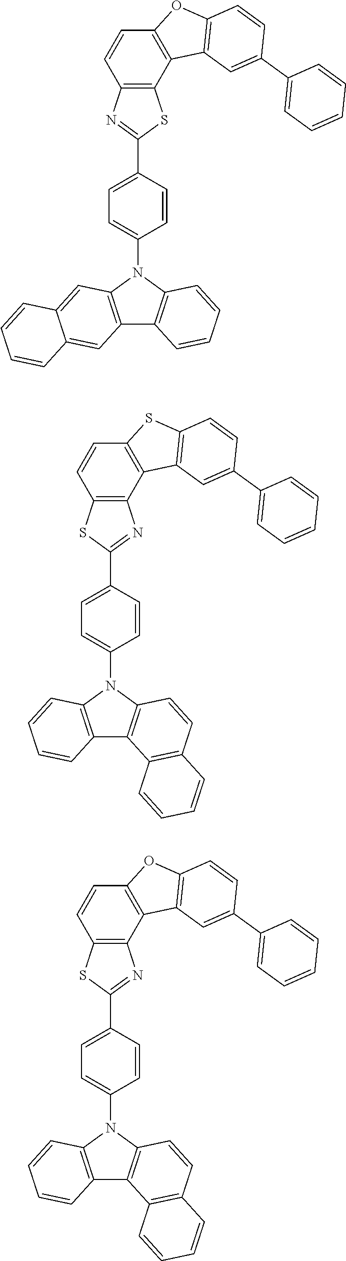

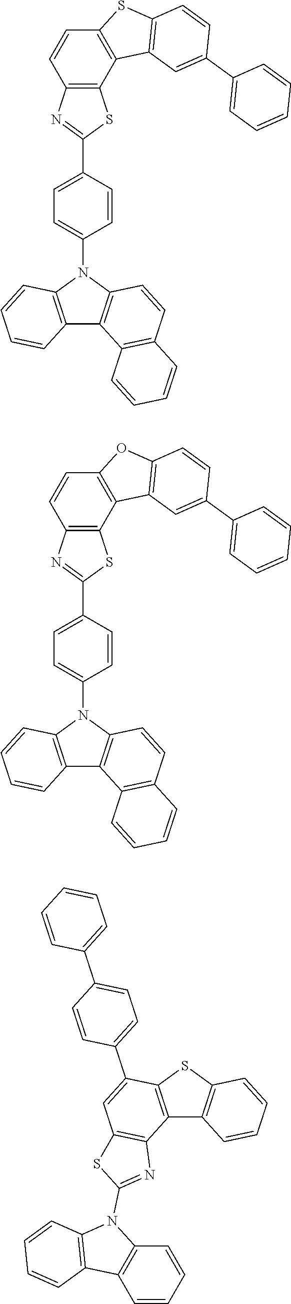

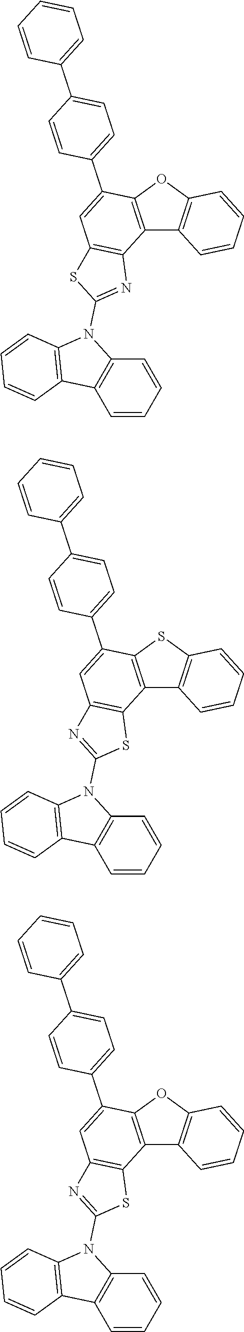 [SMILES: c1ccc(-c2ccc(-c3cc4sc(-n5c6ccccc6c6ccccc65)nc4c4c3oc3ccccc34)cc2)cc1 ; c1ccc(-c2ccc(-c3cc4nc(-n5c6ccccc6c6ccccc65)sc4c4c3sc3ccccc34)cc2)cc1 ; c1ccc(-c2ccc(-c3cc4nc(-n5c6ccccc6c6ccccc65)sc4c4c3oc3ccccc34)cc2)cc1]

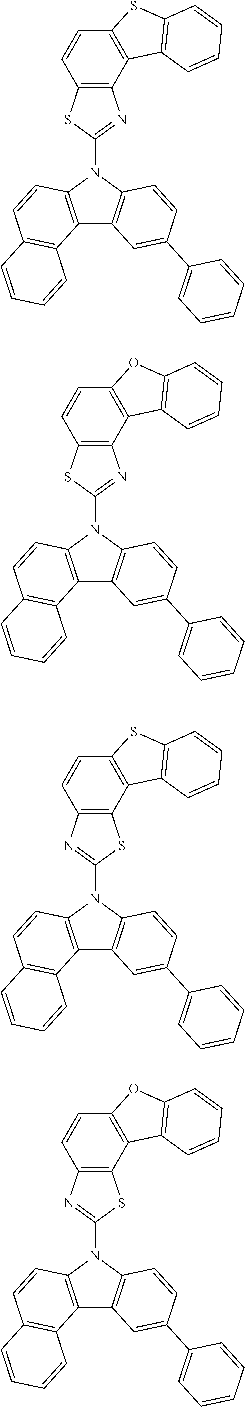

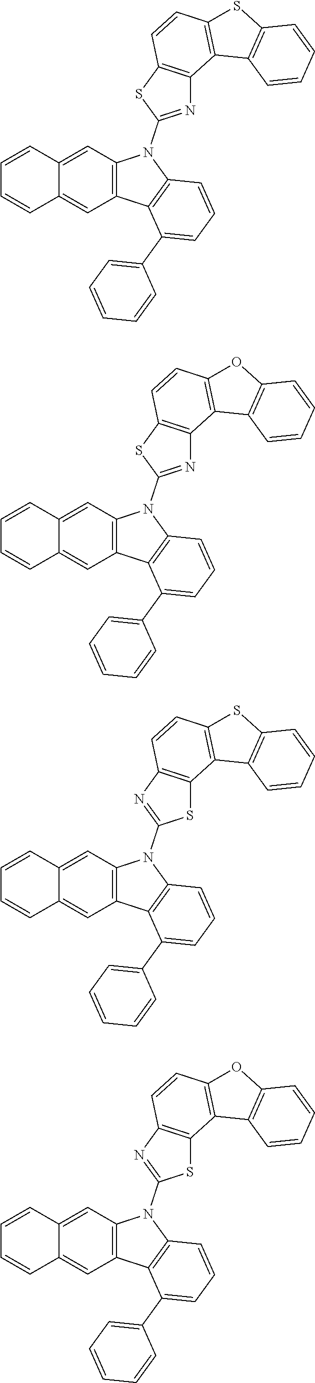 [SMILES: c1ccc(-c2cccc3c2c2cc4ccccc4cc2n3-c2nc3c(ccc4sc5ccccc5c43)s2)cc1 ; c1ccc(-c2cccc3c2c2cc4ccccc4cc2n3-c2nc3c(ccc4oc5ccccc5c43)s2)cc1 ; c1ccc(-c2cccc3c2c2cc4ccccc4cc2n3-c2nc3ccc4sc5ccccc5c4c3s2)cc1 ; c1ccc(-c2cccc3c2c2cc4ccccc4cc2n3-c2nc3ccc4oc5ccccc5c4c3s2)cc1]

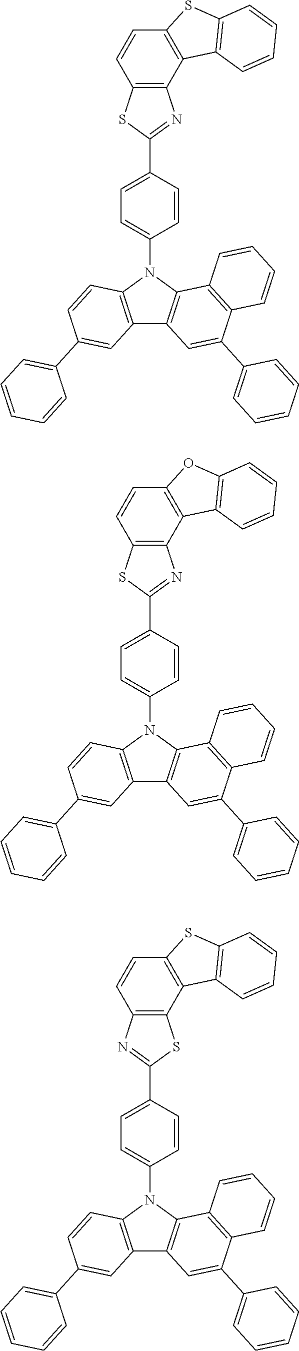

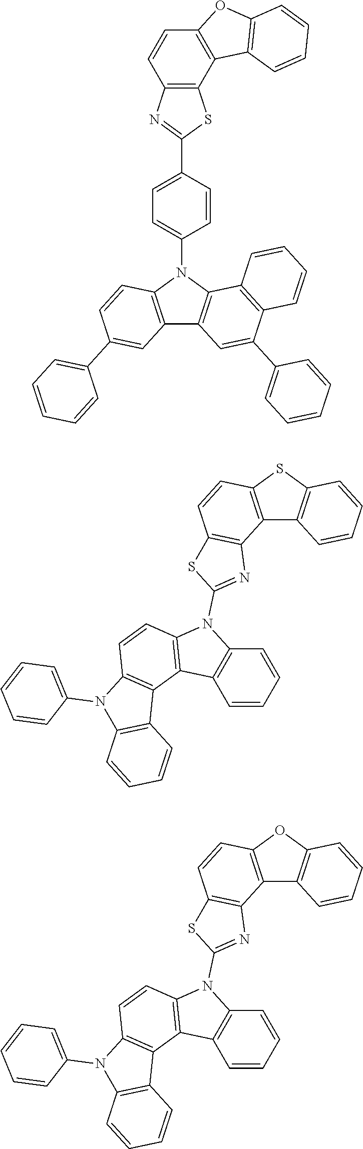

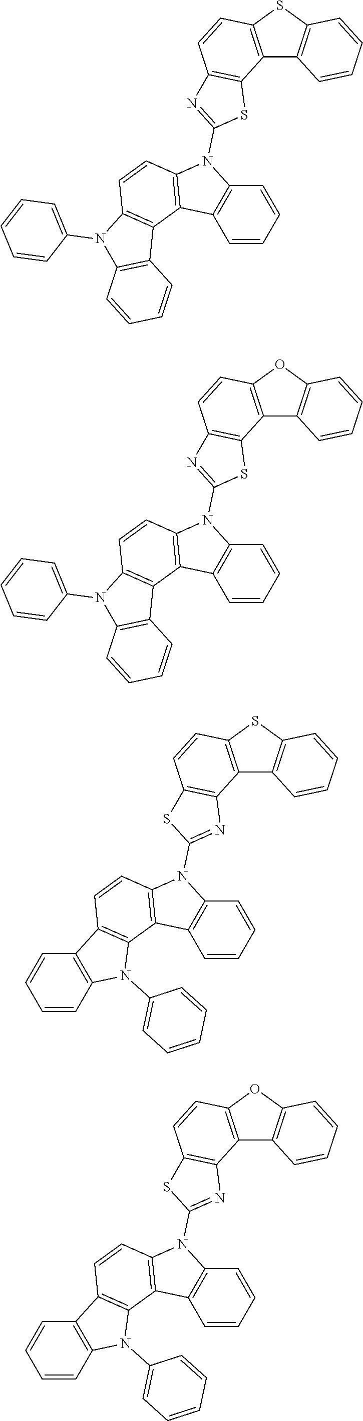 [SMILES: c1ccc(-n2c3ccccc3c3c4c5ccccc5n(-c5nc6ccc7sc8ccccc8c7c6s5)c4ccc32)cc1 ; c1ccc(-n2c3ccccc3c3c4c5ccccc5n(-c5nc6ccc7oc8ccccc8c7c6s5)c4ccc32)cc1 ; c1ccc(-n2c3ccccc3c3ccc4c(c5ccccc5n4-c4nc5c(ccc6sc7ccccc7c65)s4)c32)cc1 ; c1ccc(-n2c3ccccc3c3ccc4c(c5ccccc5n4-c4nc5c(ccc6oc7ccccc7c65)s4)c32)cc1]

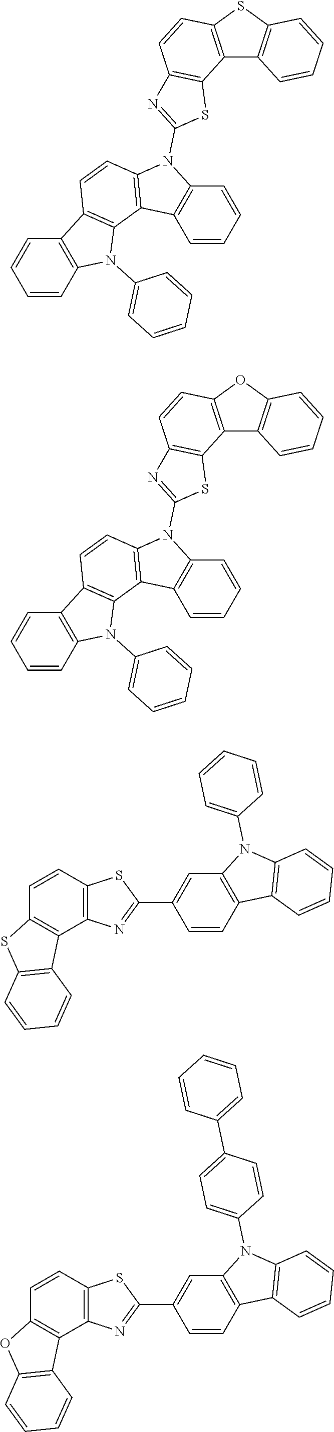 [SMILES: c1ccc(-n2c3ccccc3c3ccc4c(c5ccccc5n4-c4nc5ccc6sc7ccccc7c6c5s4)c32)cc1 ; c1ccc(-n2c3ccccc3c3ccc4c(c5ccccc5n4-c4nc5ccc6oc7ccccc7c6c5s4)c32)cc1 ; c1ccc(-n2c3ccccc3c3ccc(-c4nc5c(ccc6sc7ccccc7c65)s4)cc32)cc1 ; c1ccc(-c2ccc(-n3c4ccccc4c4ccc(-c5nc6c(ccc7oc8ccccc8c76)s5)cc43)cc2)cc1]

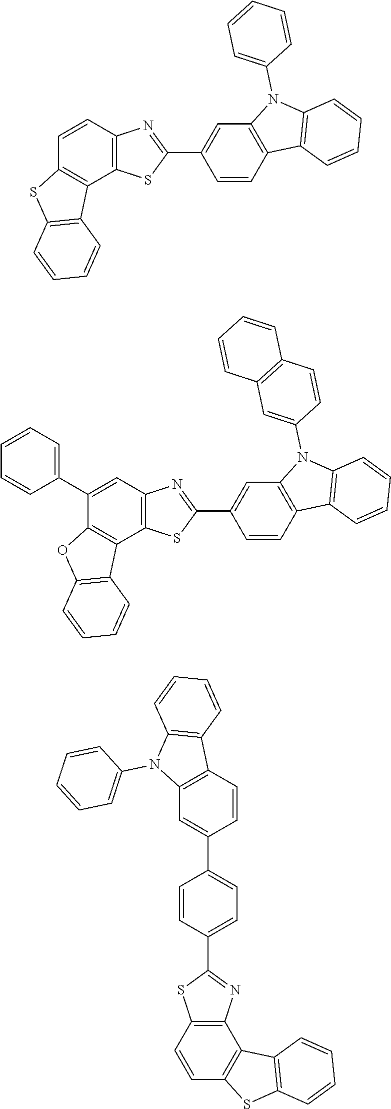 [SMILES: c1ccc(-n2c3ccccc3c3ccc(-c4nc5ccc6sc7ccccc7c6c5s4)cc32)cc1 ; c1ccc(-c2cc3nc(-c4ccc5c6ccccc6n(-c6ccc7ccccc7c6)c5c4)sc3c3c2oc2ccccc23)cc1 ; c1ccc(-n2c3ccccc3c3ccc(-c4ccc(-c5nc6c(ccc7sc8ccccc8c76)s5)cc4)cc32)cc1]

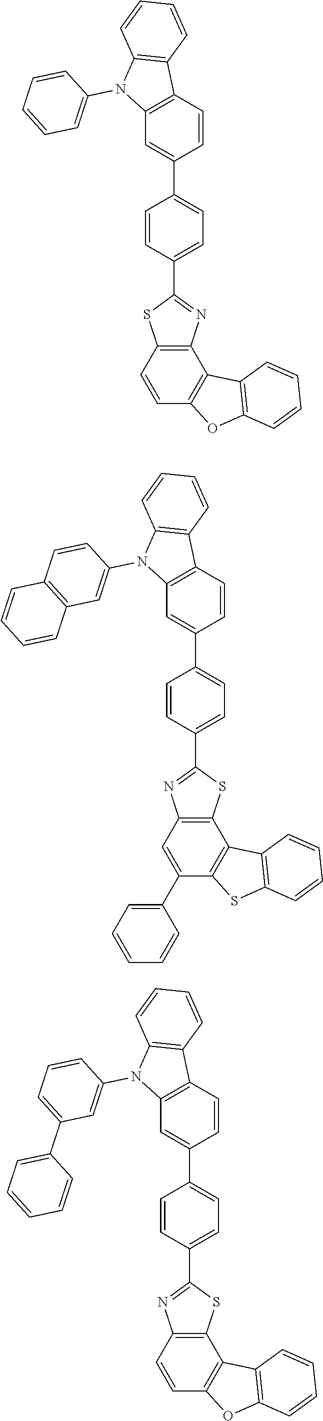

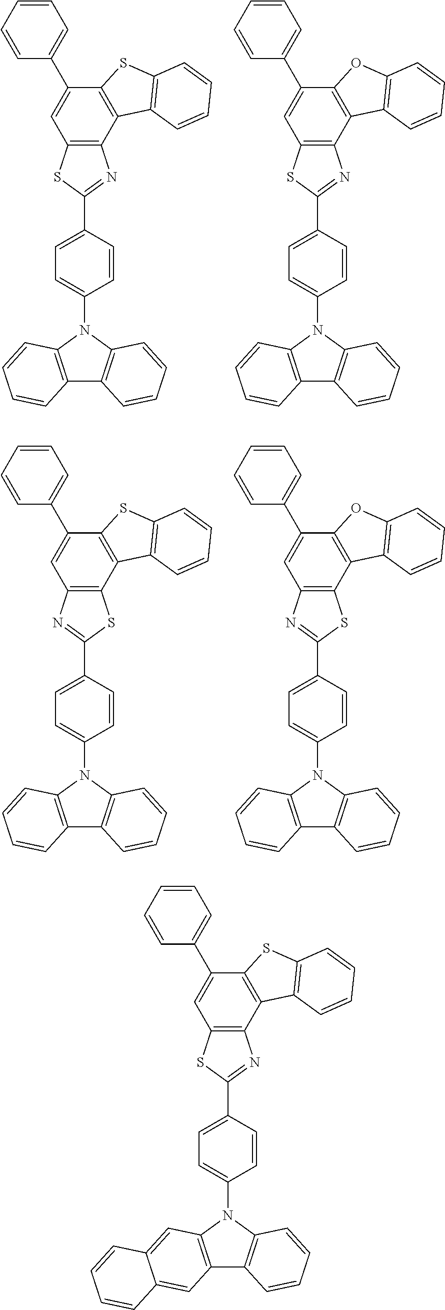

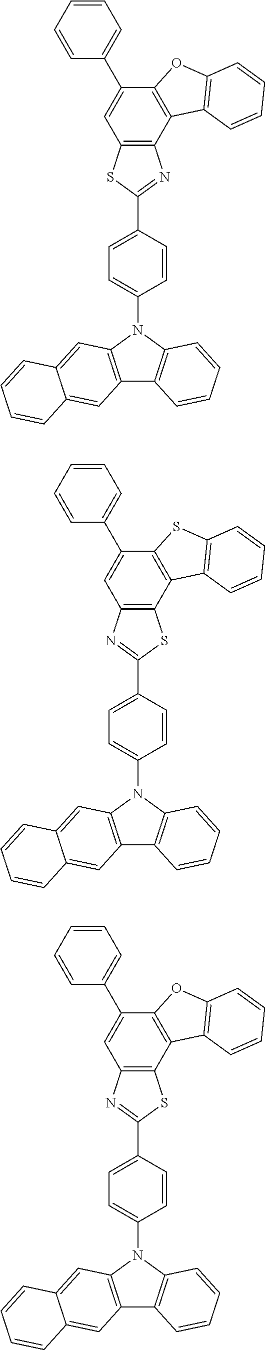 [SMILES: c1ccc(-c2cc3sc(-c4ccc(-n5c6ccccc6c6cc7ccccc7cc65)cc4)nc3c3c2oc2ccccc23)cc1 ; c1ccc(-c2cc3nc(-c4ccc(-n5c6ccccc6c6cc7ccccc7cc65)cc4)sc3c3c2sc2ccccc23)cc1 ; c1ccc(-c2cc3nc(-c4ccc(-n5c6ccccc6c6cc7ccccc7cc65)cc4)sc3c3c2oc2ccccc23)cc1]

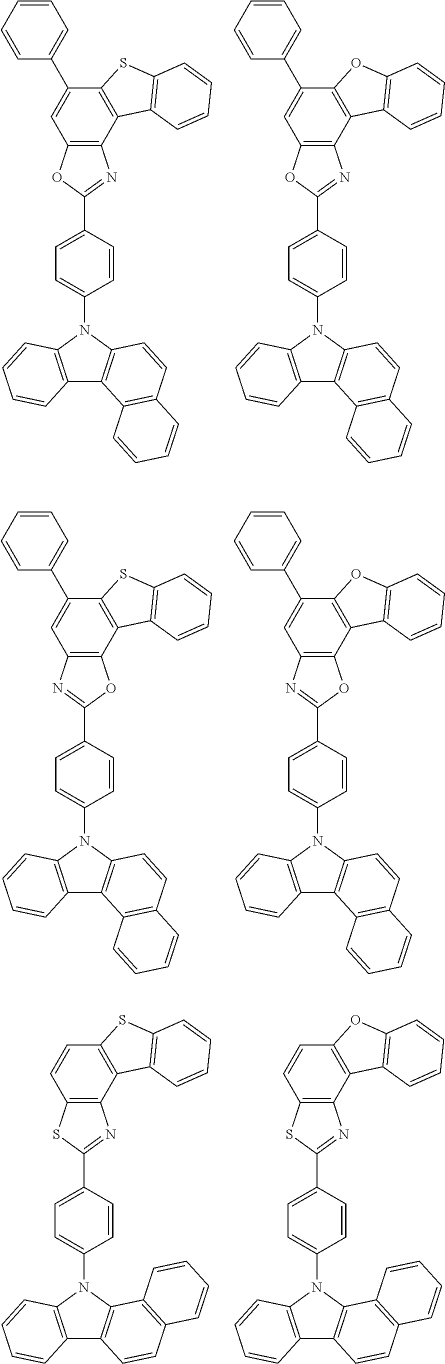

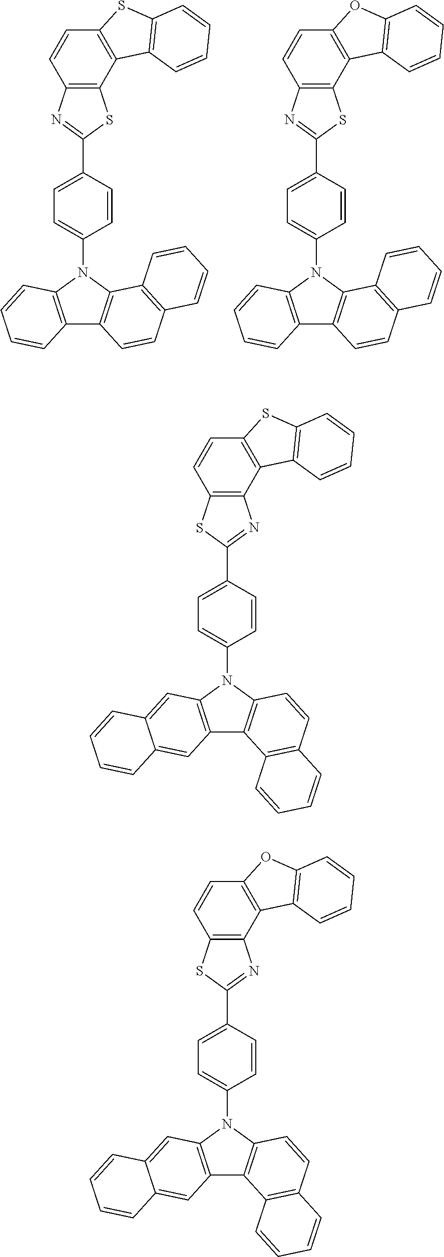

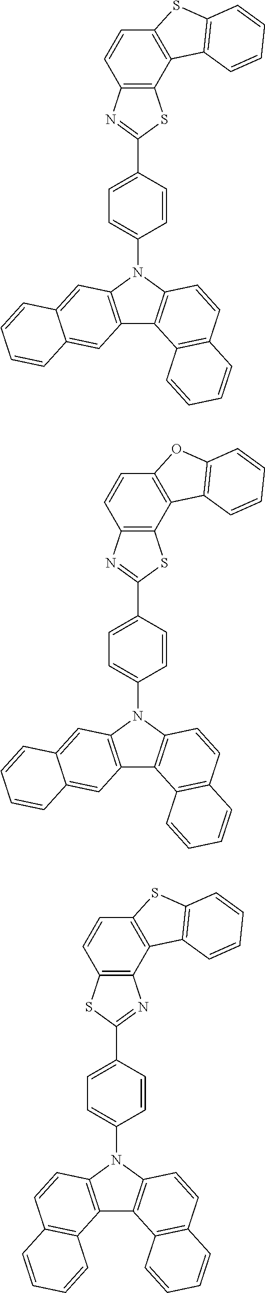

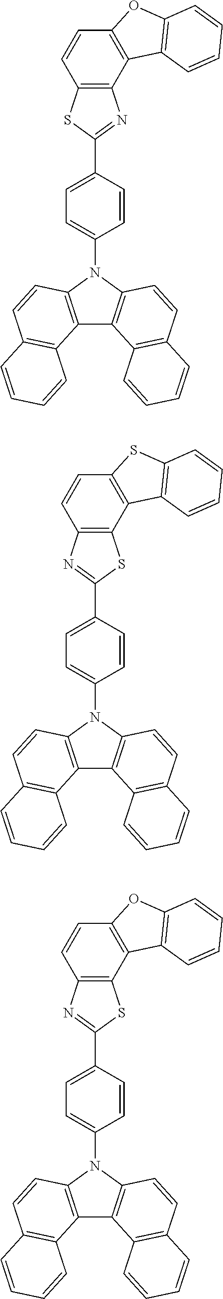

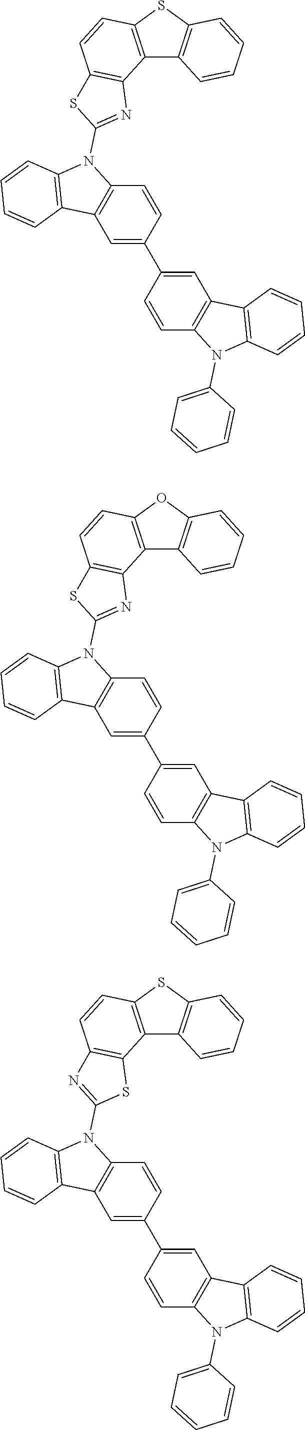 [SMILES: c1ccc(-n2c3ccccc3c3cc(-c4ccc5c(c4)c4ccccc4n5-c4nc5c(ccc6sc7ccccc7c65)s4)ccc32)cc1 ; c1ccc(-n2c3ccccc3c3cc(-c4ccc5c(c4)c4ccccc4n5-c4nc5c(ccc6oc7ccccc7c65)s4)ccc32)cc1 ; c1ccc(-n2c3ccccc3c3cc(-c4ccc5c(c4)c4ccccc4n5-c4nc5ccc6sc7ccccc7c6c5s4)ccc32)cc1]

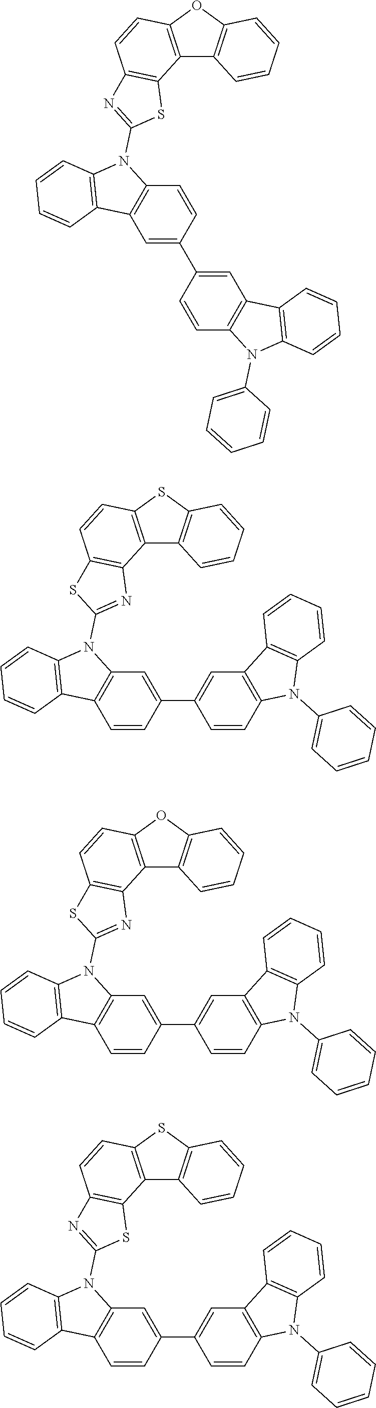 [SMILES: c1ccc(-n2c3ccccc3c3cc(-c4ccc5c(c4)c4ccccc4n5-c4nc5ccc6oc7ccccc7c6c5s4)ccc32)cc1 ; c1ccc(-n2c3ccccc3c3cc(-c4ccc5c6ccccc6n(-c6nc7c(ccc8sc9ccccc9c87)s6)c5c4)ccc32)cc1 ; c1ccc(-n2c3ccccc3c3cc(-c4ccc5c6ccccc6n(-c6nc7c(ccc8oc9ccccc9c87)s6)c5c4)ccc32)cc1 ; c1ccc(-n2c3ccccc3c3cc(-c4ccc5c6ccccc6n(-c6nc7ccc8sc9ccccc9c8c7s6)c5c4)ccc32)cc1]

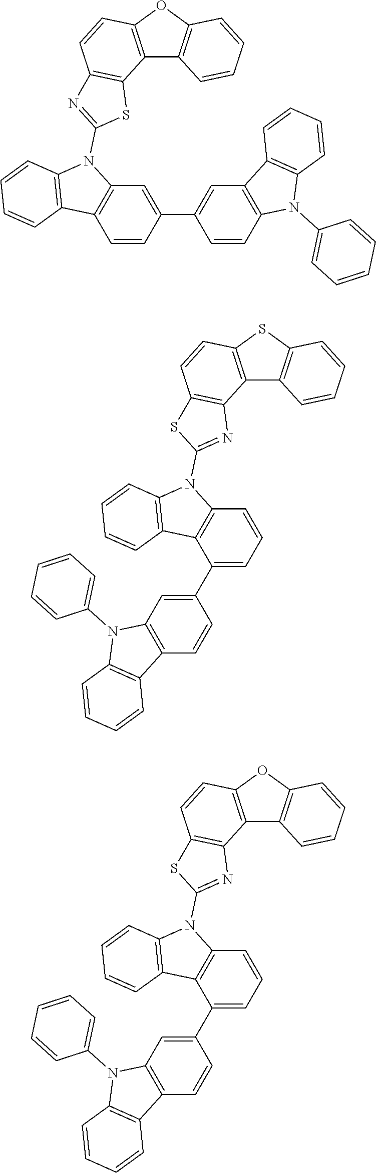 [SMILES: c1ccc(-n2c3ccccc3c3cc(-c4ccc5c6ccccc6n(-c6nc7ccc8oc9ccccc9c8c7s6)c5c4)ccc32)cc1 ; c1ccc(-n2c3ccccc3c3ccc(-c4cccc5c4c4ccccc4n5-c4nc5c(ccc6sc7ccccc7c65)s4)cc32)cc1 ; c1ccc(-n2c3ccccc3c3ccc(-c4cccc5c4c4ccccc4n5-c4nc5c(ccc6oc7ccccc7c65)s4)cc32)cc1]

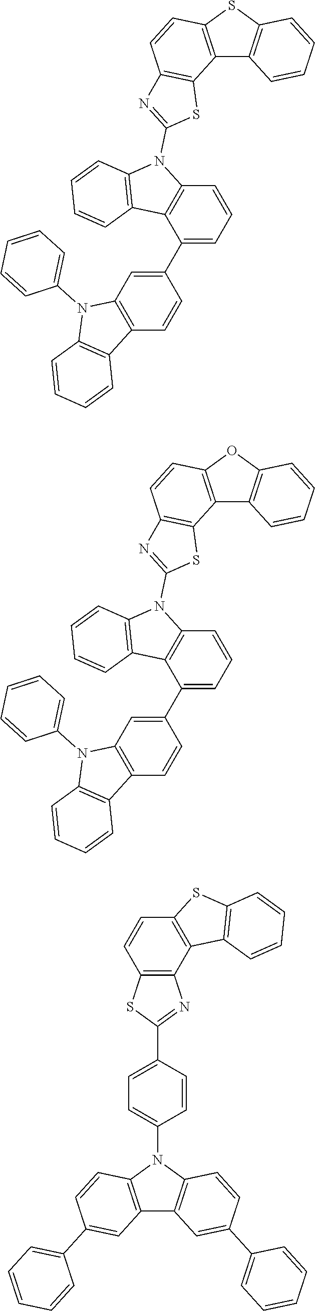 [SMILES: c1ccc(-n2c3ccccc3c3ccc(-c4cccc5c4c4ccccc4n5-c4nc5ccc6sc7ccccc7c6c5s4)cc32)cc1 ; c1ccc(-n2c3ccccc3c3ccc(-c4cccc5c4c4ccccc4n5-c4nc5ccc6oc7ccccc7c6c5s4)cc32)cc1 ; c1ccc(-c2ccc3c(c2)c2cc(-c4ccccc4)ccc2n3-c2ccc(-c3nc4c(ccc5sc6ccccc6c54)s3)cc2)cc1]

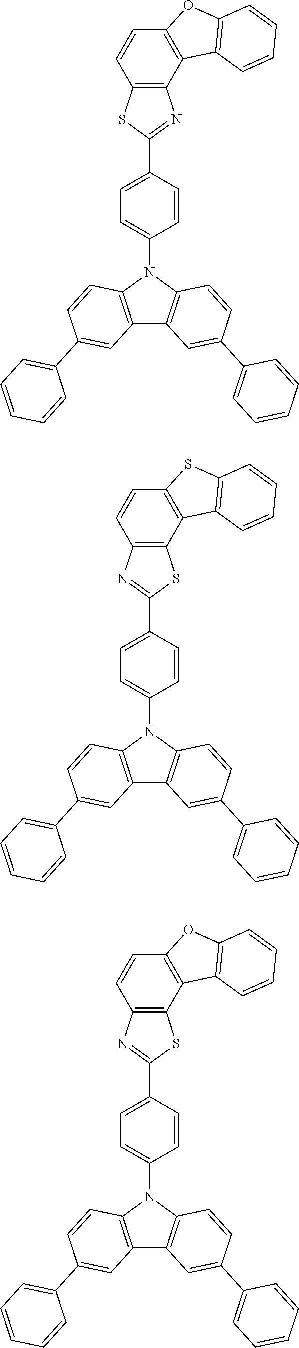

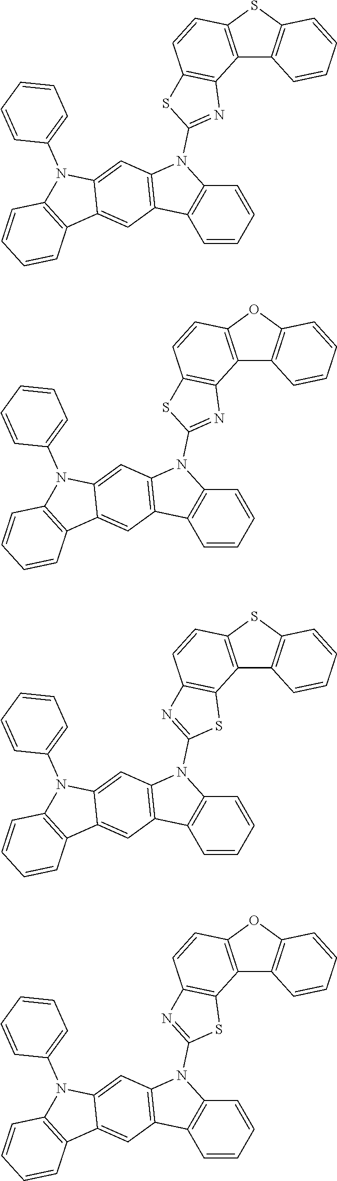 [SMILES: c1ccc(-n2c3ccccc3c3cc4c5ccccc5n(-c5nc6c(ccc7sc8ccccc8c76)s5)c4cc32)cc1 ; c1ccc(-n2c3ccccc3c3cc4c5ccccc5n(-c5nc6c(ccc7oc8ccccc8c76)s5)c4cc32)cc1 ; c1ccc(-n2c3ccccc3c3cc4c5ccccc5n(-c5nc6ccc7sc8ccccc8c7c6s5)c4cc32)cc1 ; c1ccc(-n2c3ccccc3c3cc4c5ccccc5n(-c5nc6ccc7oc8ccccc8c7c6s5)c4cc32)cc1]

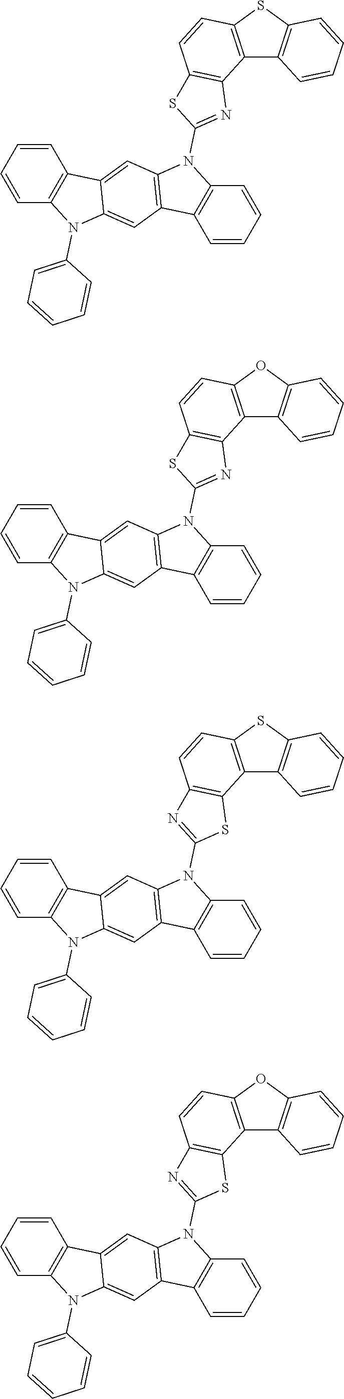 [SMILES: c1ccc(-n2c3ccccc3c3cc4c(cc32)c2ccccc2n4-c2nc3c(ccc4sc5ccccc5c43)s2)cc1 ; c1ccc(-n2c3ccccc3c3cc4c(cc32)c2ccccc2n4-c2nc3c(ccc4oc5ccccc5c43)s2)cc1 ; c1ccc(-n2c3ccccc3c3cc4c(cc32)c2ccccc2n4-c2nc3ccc4sc5ccccc5c4c3s2)cc1 ; c1ccc(-n2c3ccccc3c3cc4c(cc32)c2ccccc2n4-c2nc3ccc4oc5ccccc5c4c3s2)cc1]

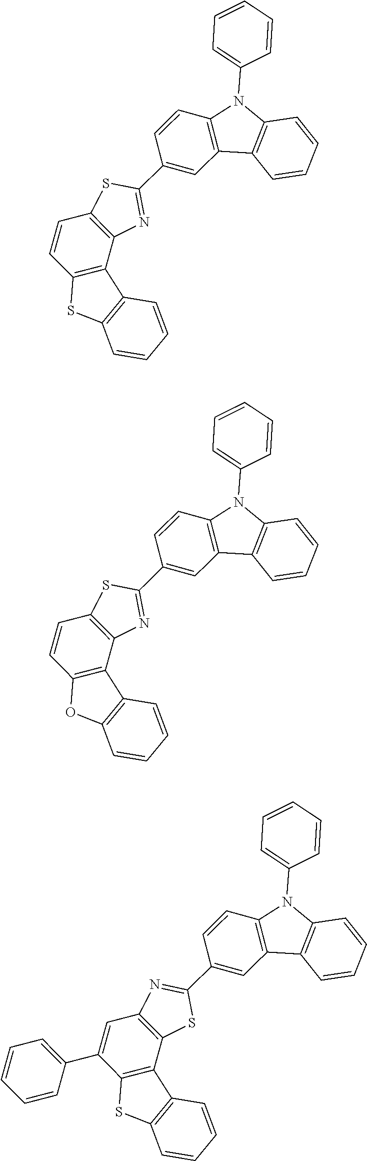 [SMILES: c1ccc(-n2c3ccccc3c3cc(-c4nc5c(ccc6sc7ccccc7c65)s4)ccc32)cc1 ; c1ccc(-n2c3ccccc3c3cc(-c4nc5c(ccc6oc7ccccc7c65)s4)ccc32)cc1 ; c1ccc(-c2cc3nc(-c4ccc5c(c4)c4ccccc4n5-c4ccccc4)sc3c3c2sc2ccccc23)cc1]

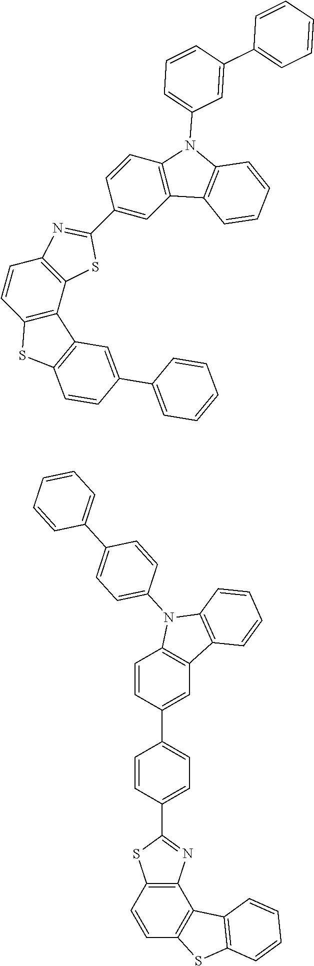 [SMILES: c1ccc(-c2cccc(-n3c4ccccc4c4cc(-c5nc6ccc7sc8ccc(-c9ccccc9)cc8c7c6s5)ccc43)c2)cc1 ; c1ccc(-c2ccc(-n3c4ccccc4c4cc(-c5ccc(-c6nc7c(ccc8sc9ccccc9c87)s6)cc5)ccc43)cc2)cc1]

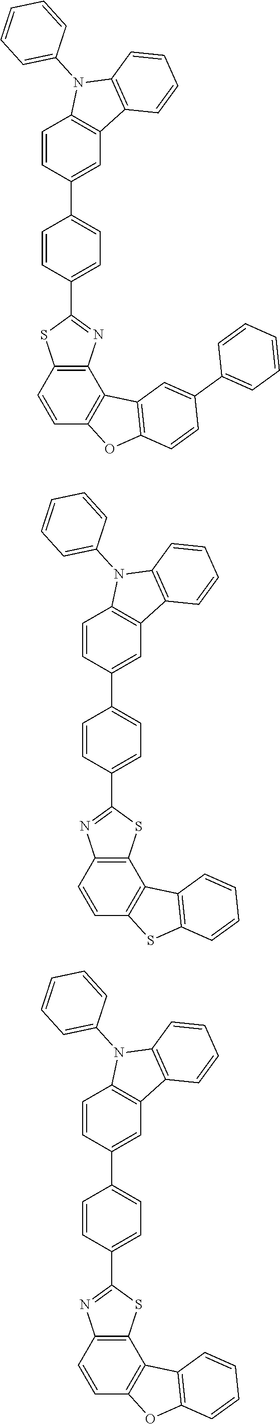 [SMILES: c1ccc(-c2ccc3oc4ccc5sc(-c6ccc(-c7ccc8c(c7)c7ccccc7n8-c7ccccc7)cc6)nc5c4c3c2)cc1 ; c1ccc(-n2c3ccccc3c3cc(-c4ccc(-c5nc6ccc7sc8ccccc8c7c6s5)cc4)ccc32)cc1 ; c1ccc(-n2c3ccccc3c3cc(-c4ccc(-c5nc6ccc7oc8ccccc8c7c6s5)cc4)ccc32)cc1]

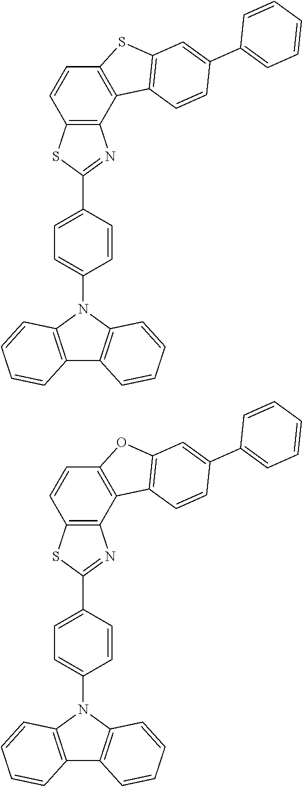 [SMILES: c1ccc(-c2ccc3c(c2)sc2ccc4sc(-c5ccc(-n6c7ccccc7c7ccccc76)cc5)nc4c23)cc1 ; c1ccc(-c2ccc3c(c2)oc2ccc4sc(-c5ccc(-n6c7ccccc7c7ccccc76)cc5)nc4c23)cc1]

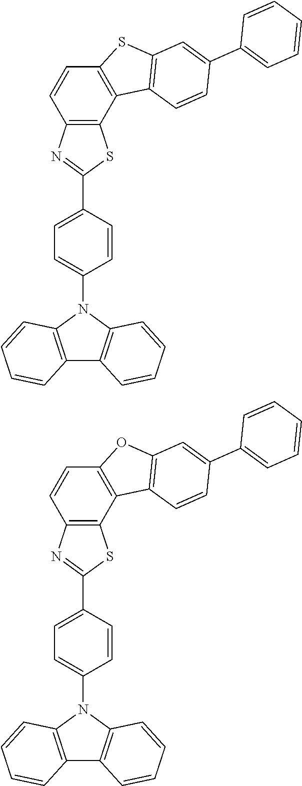 [SMILES: c1ccc(-c2ccc3c(c2)sc2ccc4nc(-c5ccc(-n6c7ccccc7c7ccccc76)cc5)sc4c23)cc1 ; c1ccc(-c2ccc3c(c2)oc2ccc4nc(-c5ccc(-n6c7ccccc7c7ccccc76)cc5)sc4c23)cc1]

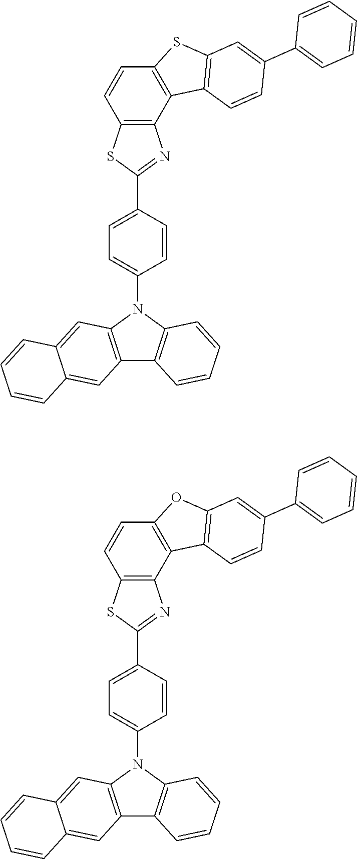 [SMILES: c1ccc(-c2ccc3c(c2)sc2ccc4sc(-c5ccc(-n6c7ccccc7c7cc8ccccc8cc76)cc5)nc4c23)cc1 ; c1ccc(-c2ccc3c(c2)oc2ccc4sc(-c5ccc(-n6c7ccccc7c7cc8ccccc8cc76)cc5)nc4c23)cc1]

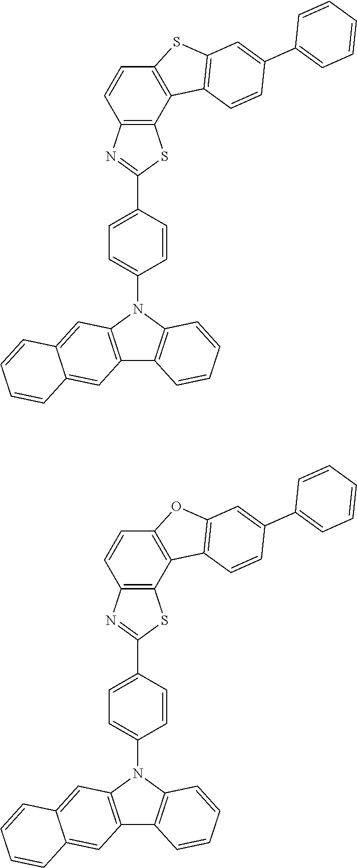 [SMILES: c1ccc(-c2ccc3c(c2)sc2ccc4nc(-c5ccc(-n6c7ccccc7c7cc8ccccc8cc76)cc5)sc4c23)cc1 ; c1ccc(-c2ccc3c(c2)oc2ccc4nc(-c5ccc(-n6c7ccccc7c7cc8ccccc8cc76)cc5)sc4c23)cc1]

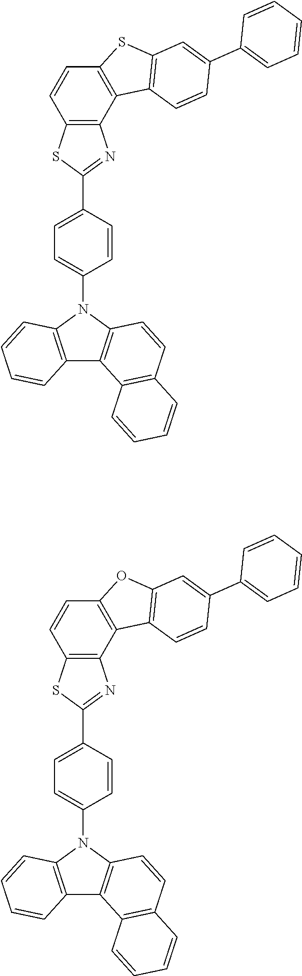 [SMILES: c1ccc(-c2ccc3c(c2)sc2ccc4sc(-c5ccc(-n6c7ccccc7c7c8ccccc8ccc76)cc5)nc4c23)cc1 ; c1ccc(-c2ccc3c(c2)oc2ccc4sc(-c5ccc(-n6c7ccccc7c7c8ccccc8ccc76)cc5)nc4c23)cc1]

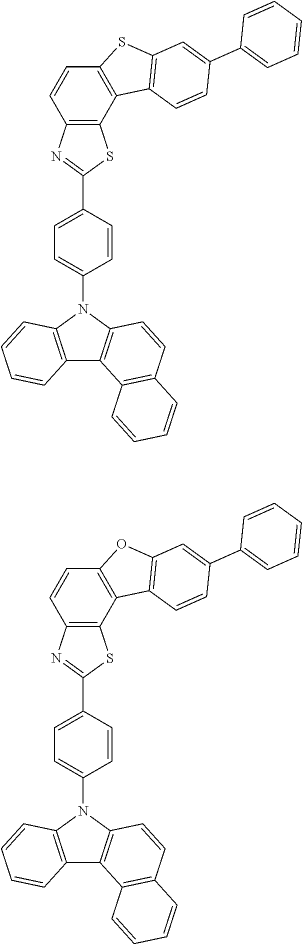 [SMILES: c1ccc(-c2ccc3c(c2)sc2ccc4nc(-c5ccc(-n6c7ccccc7c7c8ccccc8ccc76)cc5)sc4c23)cc1 ; c1ccc(-c2ccc3c(c2)oc2ccc4nc(-c5ccc(-n6c7ccccc7c7c8ccccc8ccc76)cc5)sc4c23)cc1]

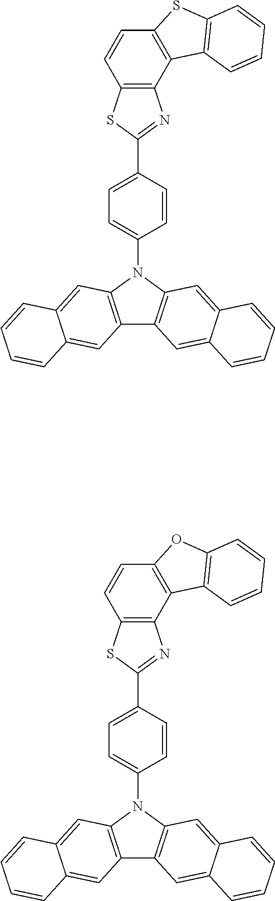 [SMILES: c1ccc2cc3c(cc2c1)c1cc2ccccc2cc1n3-c1ccc(-c2nc3c(ccc4sc5ccccc5c43)s2)cc1 ; c1ccc2cc3c(cc2c1)c1cc2ccccc2cc1n3-c1ccc(-c2nc3c(ccc4oc5ccccc5c43)s2)cc1]

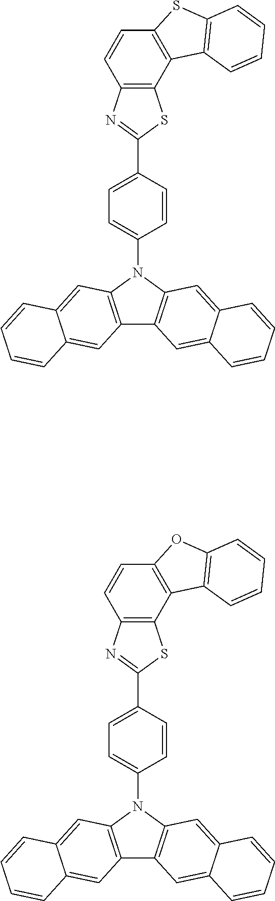 [SMILES: c1ccc2cc3c(cc2c1)c1cc2ccccc2cc1n3-c1ccc(-c2nc3ccc4sc5ccccc5c4c3s2)cc1 ; c1ccc2cc3c(cc2c1)c1cc2ccccc2cc1n3-c1ccc(-c2nc3ccc4oc5ccccc5c4c3s2)cc1]

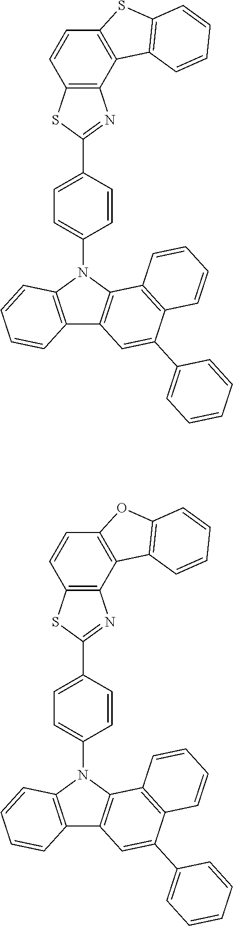 [SMILES: c1ccc(-c2cc3c4ccccc4n(-c4ccc(-c5nc6c(ccc7sc8ccccc8c76)s5)cc4)c3c3ccccc23)cc1 ; c1ccc(-c2cc3c4ccccc4n(-c4ccc(-c5nc6c(ccc7oc8ccccc8c76)s5)cc4)c3c3ccccc23)cc1]

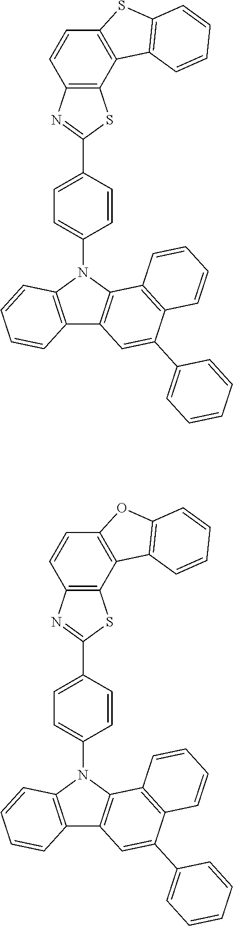 [SMILES: c1ccc(-c2cc3c4ccccc4n(-c4ccc(-c5nc6ccc7sc8ccccc8c7c6s5)cc4)c3c3ccccc23)cc1 ; c1ccc(-c2cc3c4ccccc4n(-c4ccc(-c5nc6ccc7oc8ccccc8c7c6s5)cc4)c3c3ccccc23)cc1]

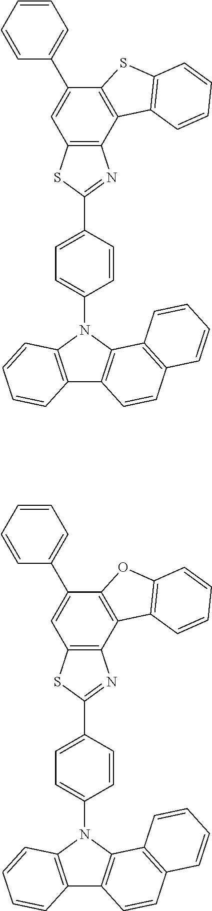 [SMILES: c1ccc(-c2cc3sc(-c4ccc(-n5c6ccccc6c6ccc7ccccc7c65)cc4)nc3c3c2sc2ccccc23)cc1 ; c1ccc(-c2cc3sc(-c4ccc(-n5c6ccccc6c6ccc7ccccc7c65)cc4)nc3c3c2oc2ccccc23)cc1]

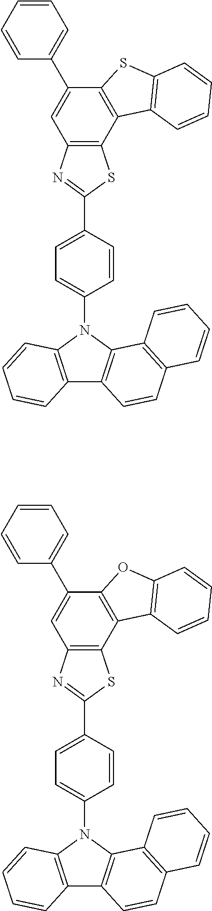 [SMILES: c1ccc(-c2cc3nc(-c4ccc(-n5c6ccccc6c6ccc7ccccc7c65)cc4)sc3c3c2sc2ccccc23)cc1 ; c1ccc(-c2cc3nc(-c4ccc(-n5c6ccccc6c6ccc7ccccc7c65)cc4)sc3c3c2oc2ccccc23)cc1]

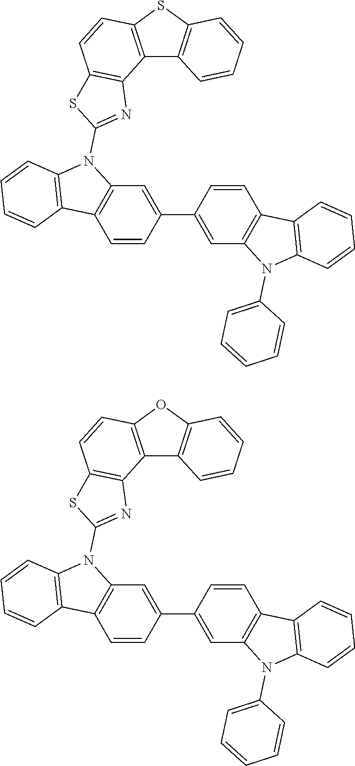 [SMILES: c1ccc(-n2c3ccccc3c3ccc(-c4ccc5c6ccccc6n(-c6nc7c(ccc8sc9ccccc9c87)s6)c5c4)cc32)cc1 ; c1ccc(-n2c3ccccc3c3ccc(-c4ccc5c6ccccc6n(-c6nc7c(ccc8oc9ccccc9c87)s6)c5c4)cc32)cc1]

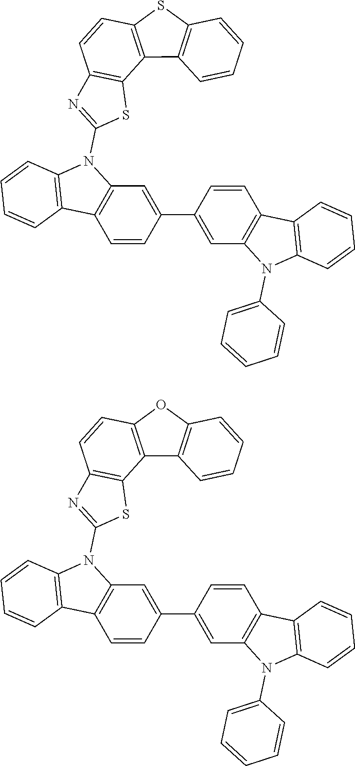 [SMILES: c1ccc(-n2c3ccccc3c3ccc(-c4ccc5c6ccccc6n(-c6nc7ccc8sc9ccccc9c8c7s6)c5c4)cc32)cc1 ; c1ccc(-n2c3ccccc3c3ccc(-c4ccc5c6ccccc6n(-c6nc7ccc8oc9ccccc9c8c7s6)c5c4)cc32)cc1]

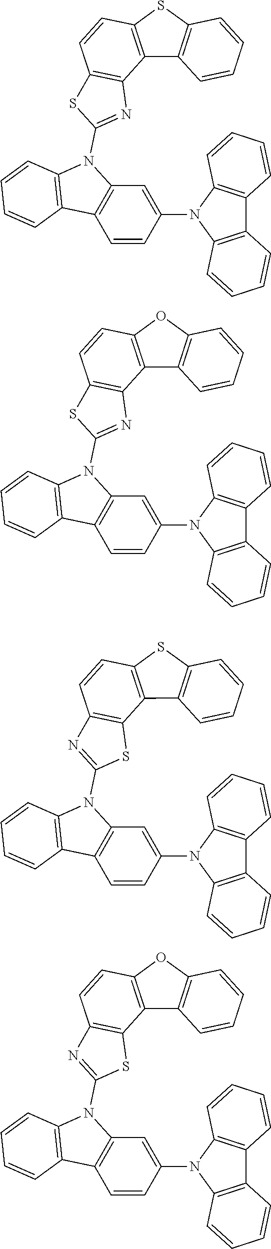

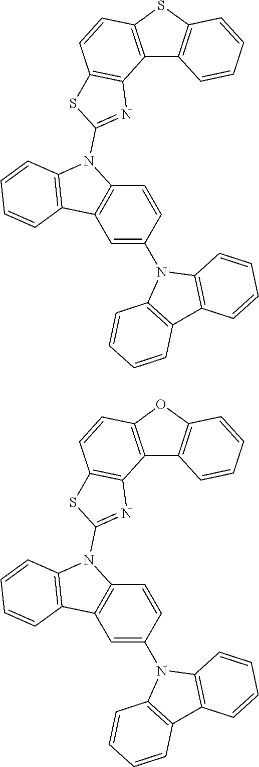

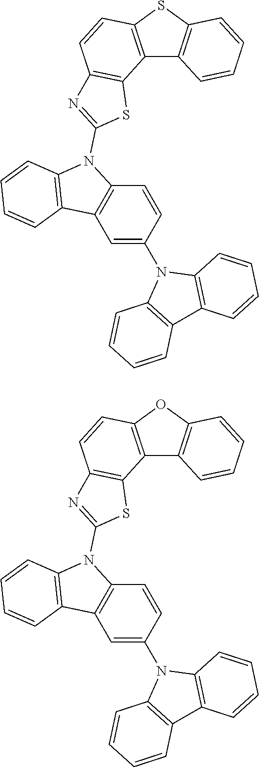

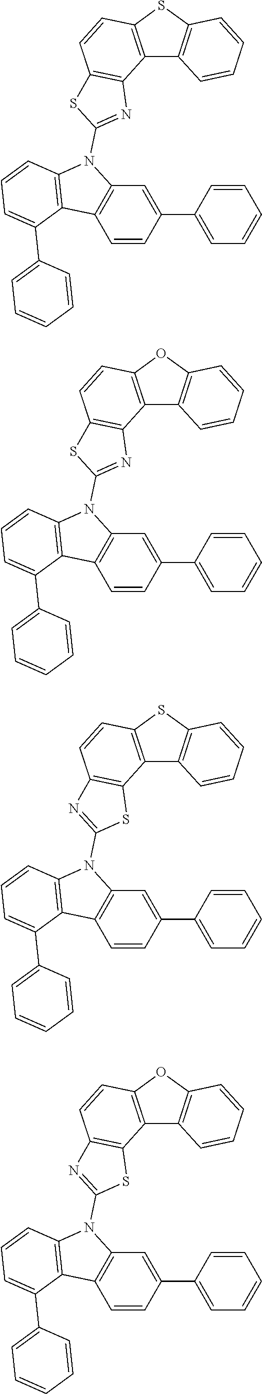 [SMILES: c1ccc(-c2ccc3c4c(-c5ccccc5)cccc4n(-c4nc5c(ccc6sc7ccccc7c65)s4)c3c2)cc1 ; c1ccc(-c2ccc3c4c(-c5ccccc5)cccc4n(-c4nc5c(ccc6oc7ccccc7c65)s4)c3c2)cc1 ; c1ccc(-c2ccc3c4c(-c5ccccc5)cccc4n(-c4nc5ccc6sc7ccccc7c6c5s4)c3c2)cc1 ; c1ccc(-c2ccc3c4c(-c5ccccc5)cccc4n(-c4nc5ccc6oc7ccccc7c6c5s4)c3c2)cc1]

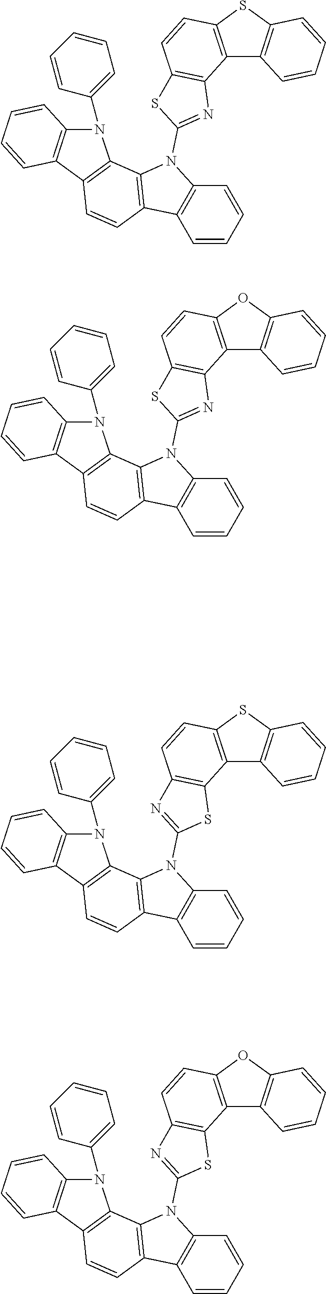 [SMILES: c1ccc(-n2c3ccccc3c3ccc4c5ccccc5n(-c5nc6c(ccc7sc8ccccc8c76)s5)c4c32)cc1 ; c1ccc(-n2c3ccccc3c3ccc4c5ccccc5n(-c5nc6c(ccc7oc8ccccc8c76)s5)c4c32)cc1 ; c1ccc(-n2c3ccccc3c3ccc4c5ccccc5n(-c5nc6ccc7sc8ccccc8c7c6s5)c4c32)cc1 ; c1ccc(-n2c3ccccc3c3ccc4c5ccccc5n(-c5nc6ccc7oc8ccccc8c7c6s5)c4c32)cc1]

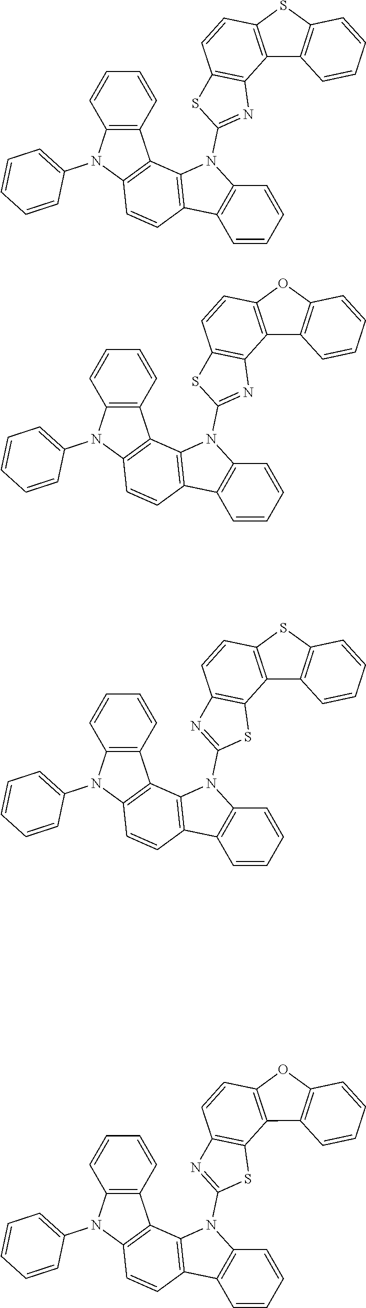

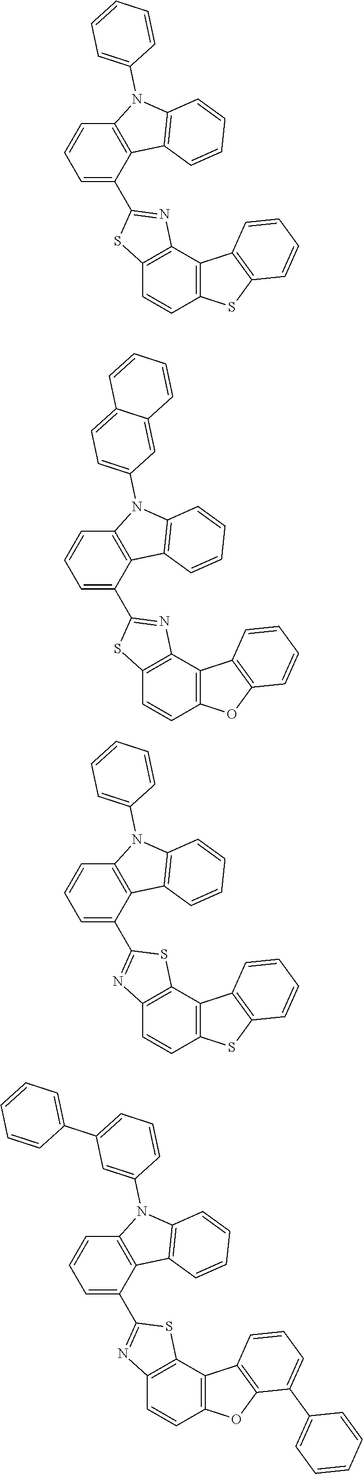

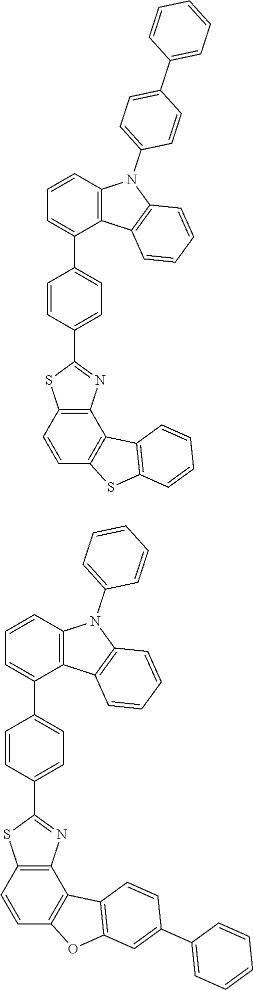

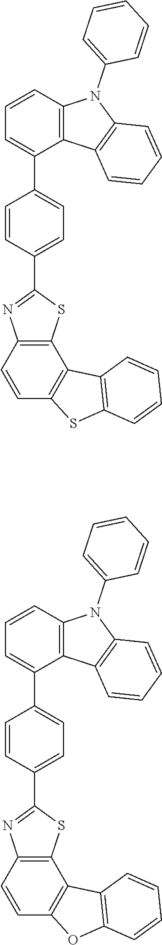 [SMILES: c1ccc(-n2c3ccccc3c3c(-c4ccc(-c5nc6ccc7sc8ccccc8c7c6s5)cc4)cccc32)cc1 ; c1ccc(-n2c3ccccc3c3c(-c4ccc(-c5nc6ccc7oc8ccccc8c7c6s5)cc4)cccc32)cc1]

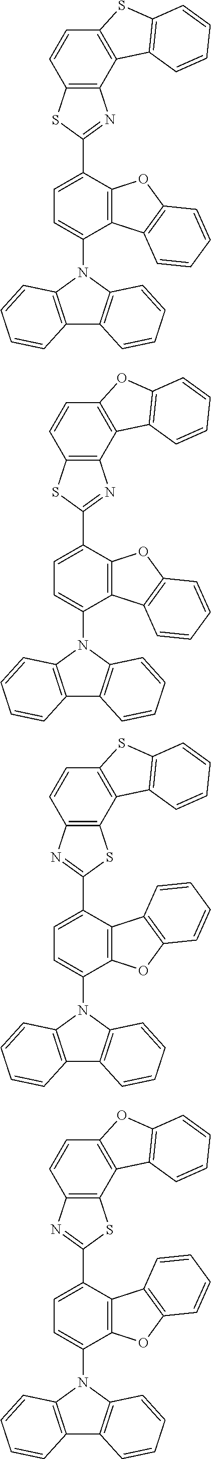

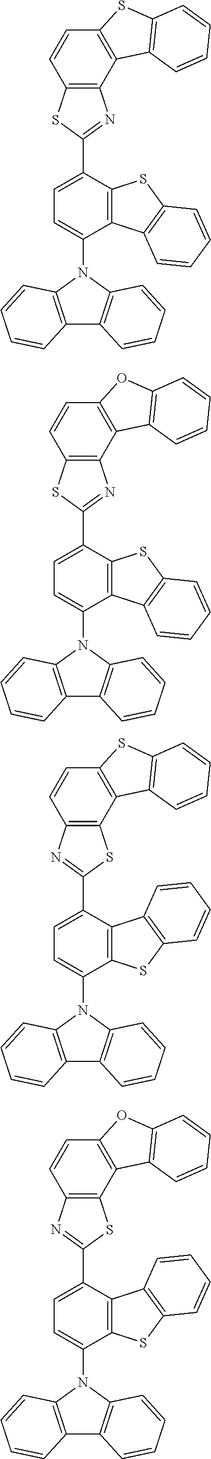

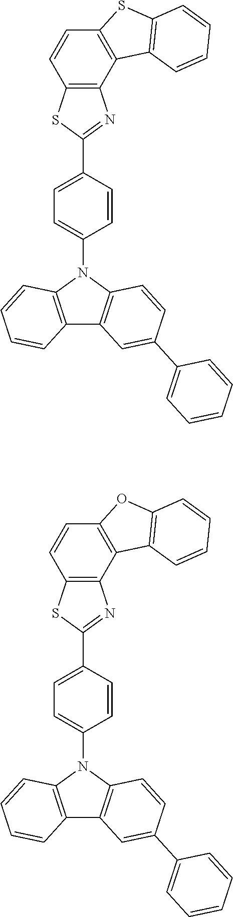 [SMILES: c1ccc(-c2ccc3c(c2)c2ccccc2n3-c2ccc(-c3nc4c(ccc5sc6ccccc6c54)s3)cc2)cc1 ; c1ccc(-c2ccc3c(c2)c2ccccc2n3-c2ccc(-c3nc4c(ccc5oc6ccccc6c54)s3)cc2)cc1]

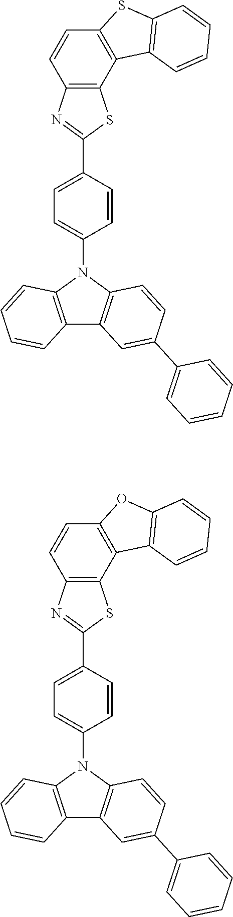 [SMILES: c1ccc(-c2ccc3c(c2)c2ccccc2n3-c2ccc(-c3nc4ccc5sc6ccccc6c5c4s3)cc2)cc1 ; c1ccc(-c2ccc3c(c2)c2ccccc2n3-c2ccc(-c3nc4ccc5oc6ccccc6c5c4s3)cc2)cc1]

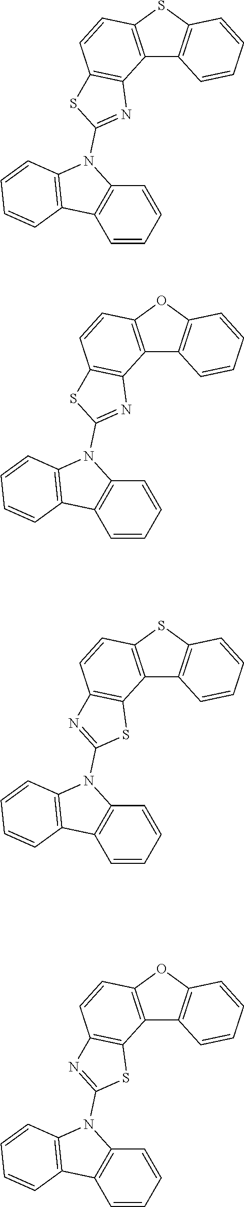 [SMILES: c1ccc2c(c1)sc1ccc3sc(-n4c5ccccc5c5ccccc54)nc3c12 ; c1ccc2c(c1)oc1ccc3sc(-n4c5ccccc5c5ccccc54)nc3c12 ; c1ccc2c(c1)sc1ccc3nc(-n4c5ccccc5c5ccccc54)sc3c12 ; c1ccc2c(c1)oc1ccc3nc(-n4c5ccccc5c5ccccc54)sc3c12]

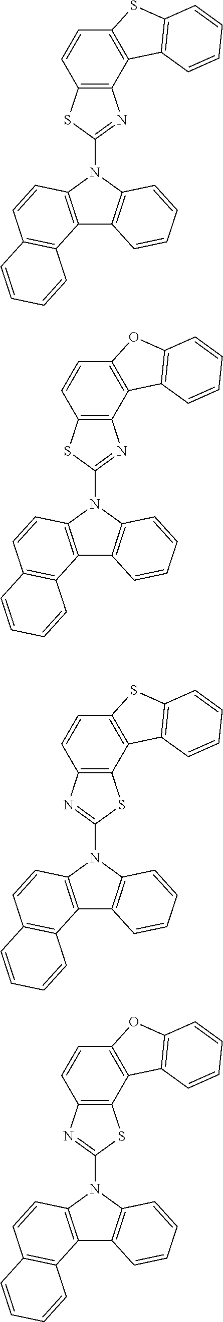 [SMILES: c1ccc2c(c1)ccc1c2c2ccccc2n1-c1nc2c(ccc3sc4ccccc4c32)s1 ; c1ccc2c(c1)ccc1c2c2ccccc2n1-c1nc2c(ccc3oc4ccccc4c32)s1 ; c1ccc2c(c1)ccc1c2c2ccccc2n1-c1nc2ccc3sc4ccccc4c3c2s1 ; c1ccc2c(c1)ccc1c2c2ccccc2n1-c1nc2ccc3oc4ccccc4c3c2s1]

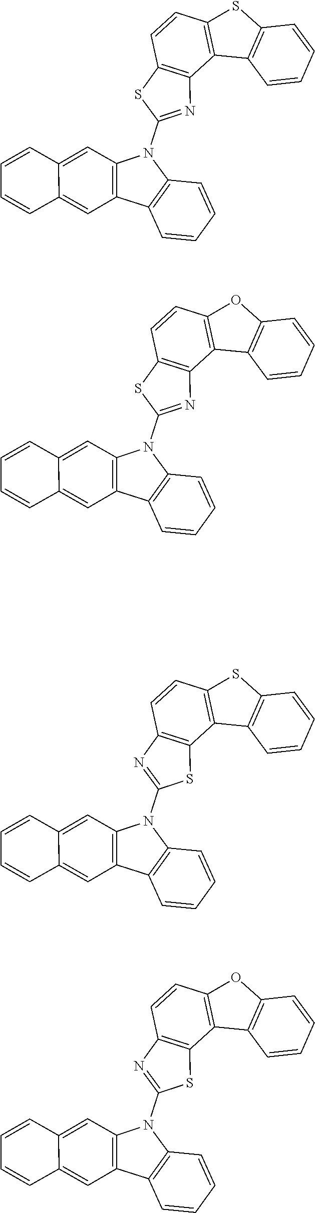 [SMILES: c1ccc2cc3c(cc2c1)c1ccccc1n3-c1nc2c(ccc3sc4ccccc4c32)s1 ; c1ccc2cc3c(cc2c1)c1ccccc1n3-c1nc2c(ccc3oc4ccccc4c32)s1 ; c1ccc2cc3c(cc2c1)c1ccccc1n3-c1nc2ccc3sc4ccccc4c3c2s1 ; c1ccc2cc3c(cc2c1)c1ccccc1n3-c1nc2ccc3oc4ccccc4c3c2s1]

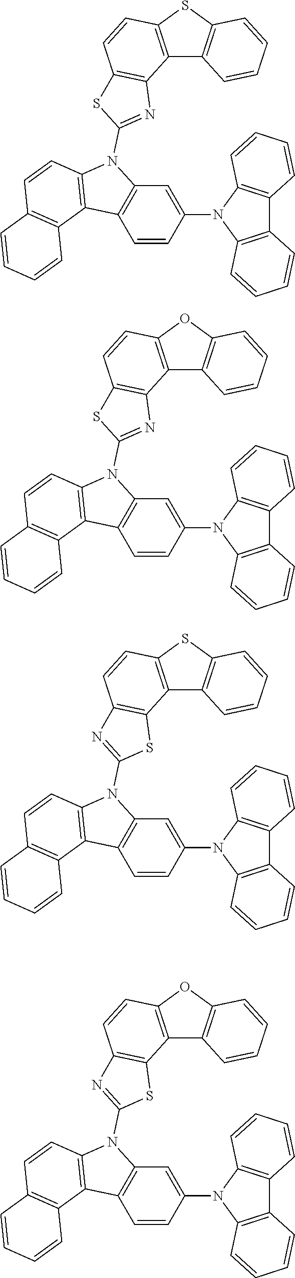

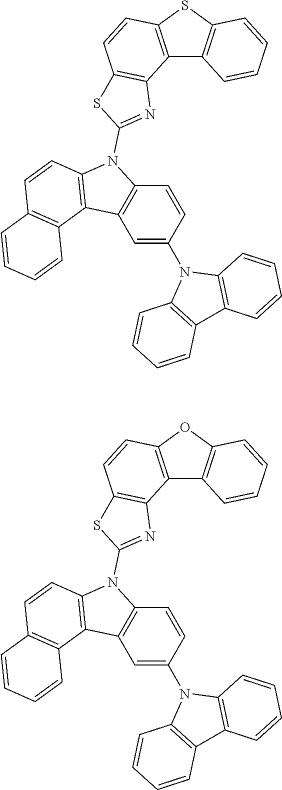 [SMILES: c1ccc2c(c1)ccc1c2c2cc(-n3c4ccccc4c4ccccc43)ccc2n1-c1nc2c(ccc3sc4ccccc4c32)s1 ; c1ccc2c(c1)ccc1c2c2cc(-n3c4ccccc4c4ccccc43)ccc2n1-c1nc2c(ccc3oc4ccccc4c32)s1]

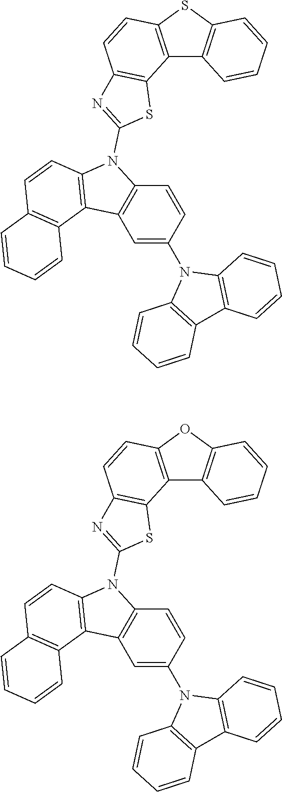 [SMILES: c1ccc2c(c1)ccc1c2c2cc(-n3c4ccccc4c4ccccc43)ccc2n1-c1nc2ccc3sc4ccccc4c3c2s1 ; c1ccc2c(c1)ccc1c2c2cc(-n3c4ccccc4c4ccccc43)ccc2n1-c1nc2ccc3oc4ccccc4c3c2s1]

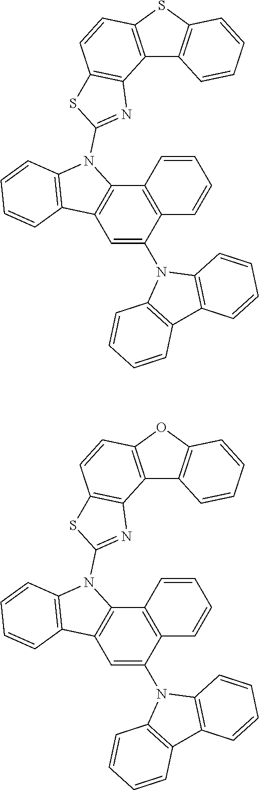 [SMILES: c1ccc2c(c1)sc1ccc3sc(-n4c5ccccc5c5cc(-n6c7ccccc7c7ccccc76)c6ccccc6c54)nc3c12 ; c1ccc2c(c1)oc1ccc3sc(-n4c5ccccc5c5cc(-n6c7ccccc7c7ccccc76)c6ccccc6c54)nc3c12]

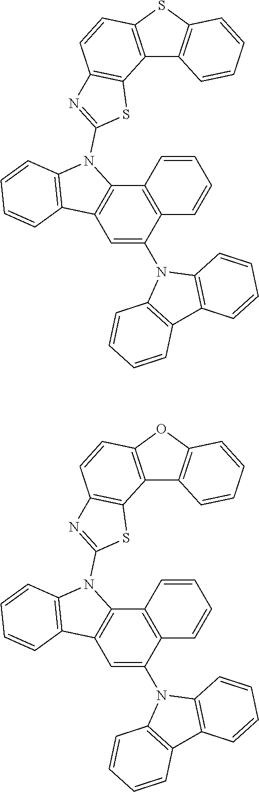 [SMILES: c1ccc2c(c1)sc1ccc3nc(-n4c5ccccc5c5cc(-n6c7ccccc7c7ccccc76)c6ccccc6c54)sc3c12 ; c1ccc2c(c1)oc1ccc3nc(-n4c5ccccc5c5cc(-n6c7ccccc7c7ccccc76)c6ccccc6c54)sc3c12]

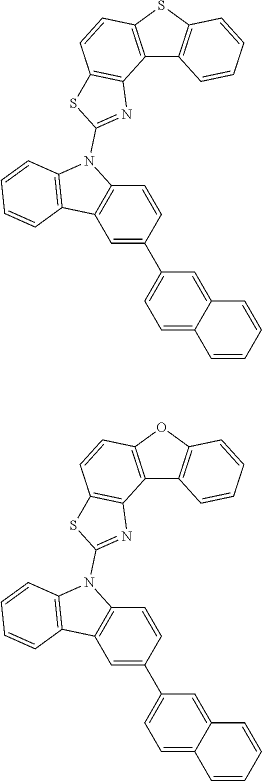 [SMILES: c1ccc2cc(-c3ccc4c(c3)c3ccccc3n4-c3nc4c(ccc5sc6ccccc6c54)s3)ccc2c1 ; c1ccc2cc(-c3ccc4c(c3)c3ccccc3n4-c3nc4c(ccc5oc6ccccc6c54)s3)ccc2c1]

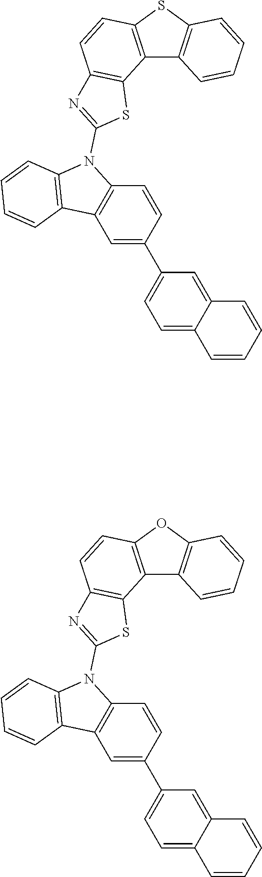 [SMILES: c1ccc2cc(-c3ccc4c(c3)c3ccccc3n4-c3nc4ccc5sc6ccccc6c5c4s3)ccc2c1 ; c1ccc2cc(-c3ccc4c(c3)c3ccccc3n4-c3nc4ccc5oc6ccccc6c5c4s3)ccc2c1]

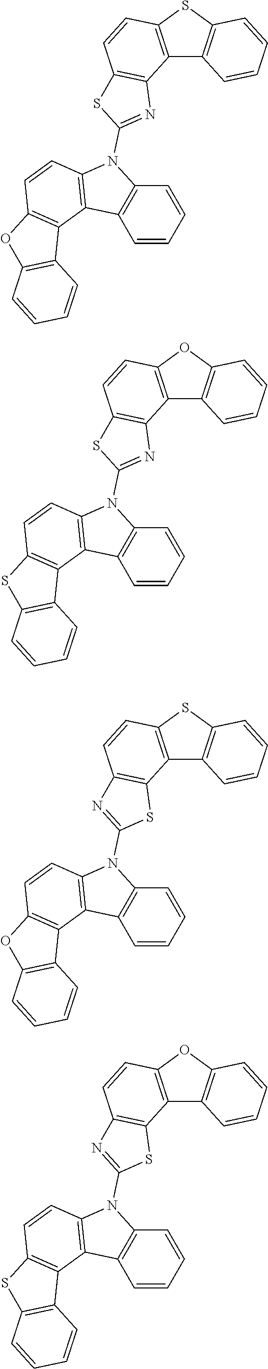 [SMILES: c1ccc2c(c1)oc1ccc3c(c4ccccc4n3-c3nc4c(ccc5sc6ccccc6c54)s3)c12 ; c1ccc2c(c1)oc1ccc3sc(-n4c5ccccc5c5c6c(ccc54)sc4ccccc46)nc3c12 ; c1ccc2c(c1)oc1ccc3c(c4ccccc4n3-c3nc4ccc5sc6ccccc6c5c4s3)c12 ; c1ccc2c(c1)oc1ccc3nc(-n4c5ccccc5c5c6c(ccc54)sc4ccccc46)sc3c12]

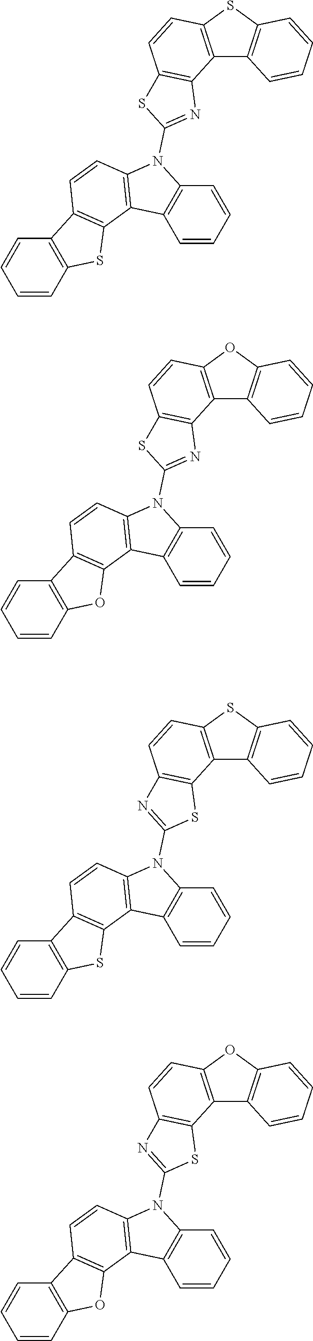 [SMILES: c1ccc2c(c1)sc1c2ccc2c1c1ccccc1n2-c1nc2c(ccc3sc4ccccc4c32)s1 ; c1ccc2c(c1)oc1c2ccc2c1c1ccccc1n2-c1nc2c(ccc3oc4ccccc4c32)s1 ; c1ccc2c(c1)sc1c2ccc2c1c1ccccc1n2-c1nc2ccc3sc4ccccc4c3c2s1 ; c1ccc2c(c1)oc1c2ccc2c1c1ccccc1n2-c1nc2ccc3oc4ccccc4c3c2s1]

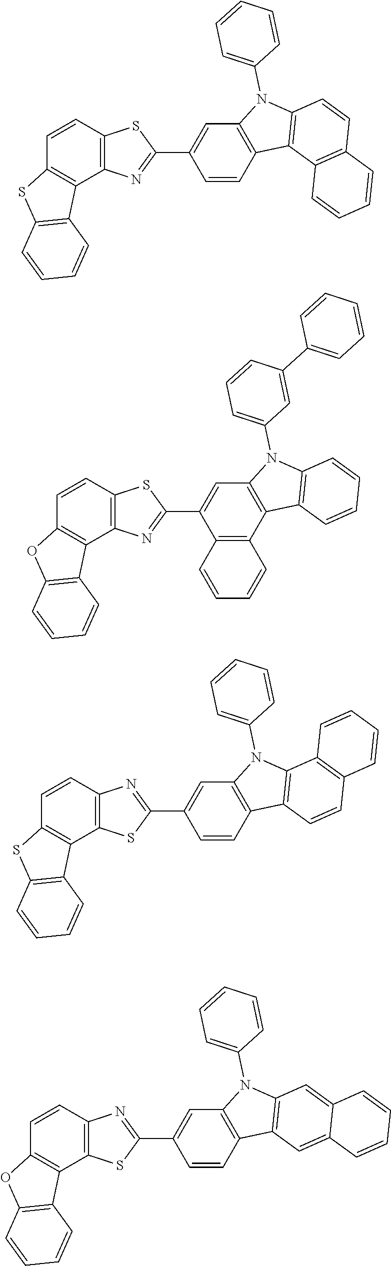 [SMILES: c1ccc(-n2c3cc(-c4nc5c(ccc6sc7ccccc7c65)s4)ccc3c3c4ccccc4ccc32)cc1 ; c1ccc(-c2cccc(-n3c4ccccc4c4c5ccccc5c(-c5nc6c(ccc7oc8ccccc8c76)s5)cc43)c2)cc1 ; c1ccc(-n2c3cc(-c4nc5ccc6sc7ccccc7c6c5s4)ccc3c3ccc4ccccc4c32)cc1 ; c1ccc(-n2c3cc(-c4nc5ccc6oc7ccccc7c6c5s4)ccc3c3cc4ccccc4cc32)cc1]

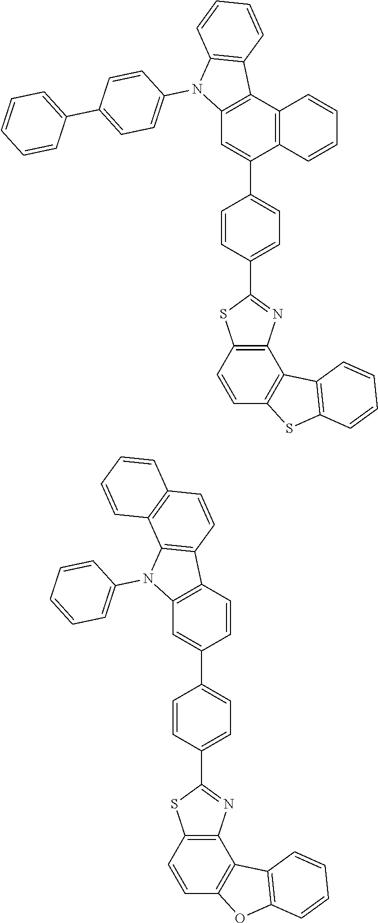 [SMILES: c1ccc(-c2ccc(-n3c4ccccc4c4c5ccccc5c(-c5ccc(-c6nc7c(ccc8sc9ccccc9c87)s6)cc5)cc43)cc2)cc1 ; c1ccc(-n2c3cc(-c4ccc(-c5nc6c(ccc7oc8ccccc8c76)s5)cc4)ccc3c3ccc4ccccc4c32)cc1]

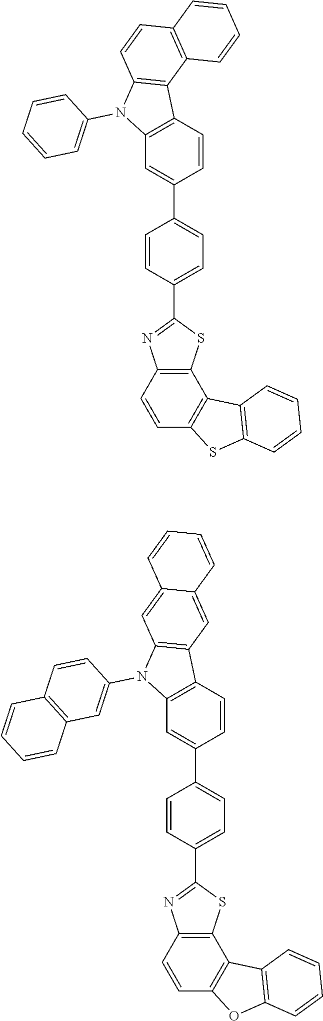 [SMILES: c1ccc(-n2c3cc(-c4ccc(-c5nc6ccc7sc8ccccc8c7c6s5)cc4)ccc3c3c4ccccc4ccc32)cc1 ; c1ccc2cc(-n3c4cc(-c5ccc(-c6nc7ccc8oc9ccccc9c8c7s6)cc5)ccc4c4cc5ccccc5cc43)ccc2c1]

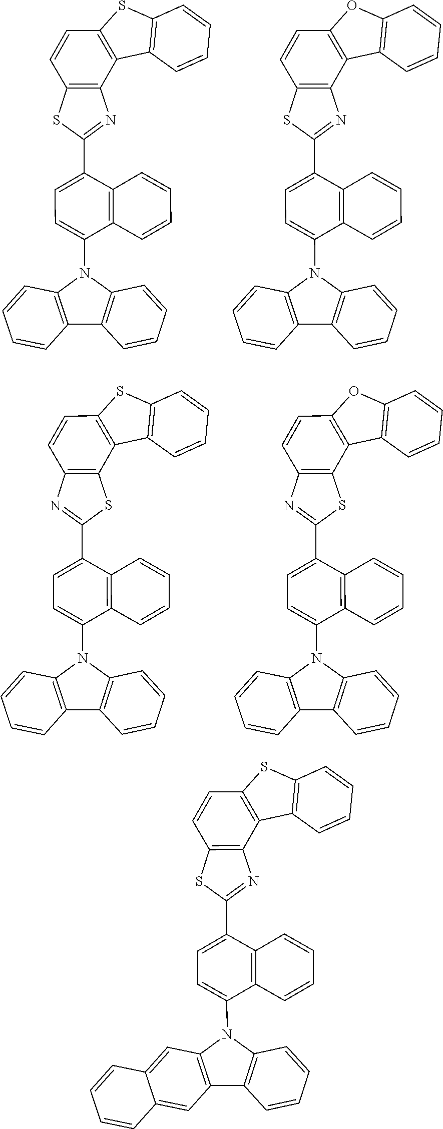

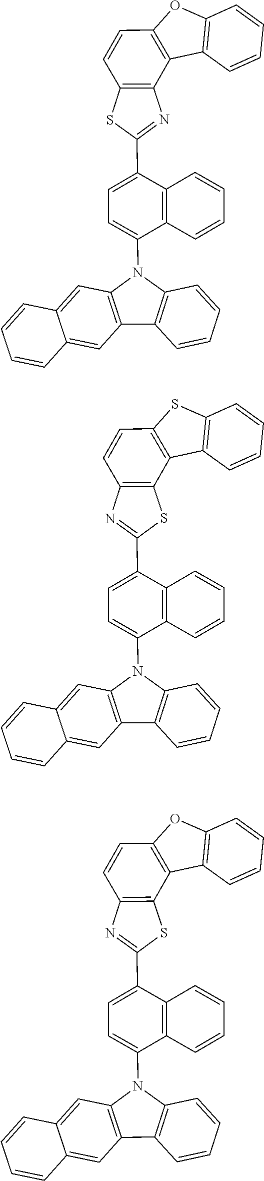 [SMILES: c1ccc2cc3c(cc2c1)c1ccccc1n3-c1ccc(-c2nc3c(ccc4oc5ccccc5c43)s2)c2ccccc12 ; c1ccc2cc3c(cc2c1)c1ccccc1n3-c1ccc(-c2nc3ccc4sc5ccccc5c4c3s2)c2ccccc12 ; c1ccc2cc3c(cc2c1)c1ccccc1n3-c1ccc(-c2nc3ccc4oc5ccccc5c4c3s2)c2ccccc12]

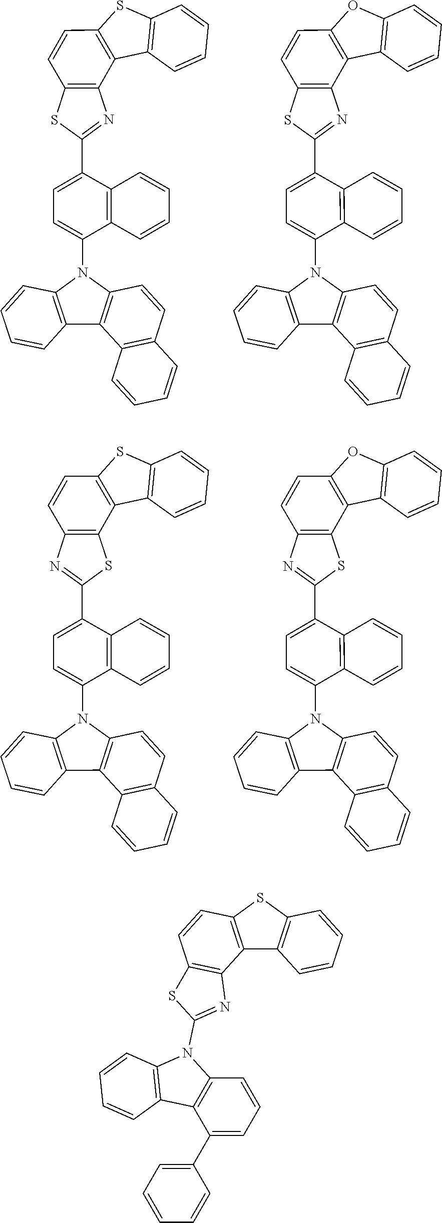 [SMILES: c1ccc2c(c1)ccc1c2c2ccccc2n1-c1ccc(-c2nc3c(ccc4sc5ccccc5c43)s2)c2ccccc12 ; c1ccc2c(c1)ccc1c2c2ccccc2n1-c1ccc(-c2nc3c(ccc4oc5ccccc5c43)s2)c2ccccc12 ; c1ccc2c(c1)ccc1c2c2ccccc2n1-c1ccc(-c2nc3ccc4sc5ccccc5c4c3s2)c2ccccc12 ; c1ccc2c(c1)ccc1c2c2ccccc2n1-c1ccc(-c2nc3ccc4oc5ccccc5c4c3s2)c2ccccc12 ; c1ccc(-c2cccc3c2c2ccccc2n3-c2nc3c(ccc4sc5ccccc5c43)s2)cc1]

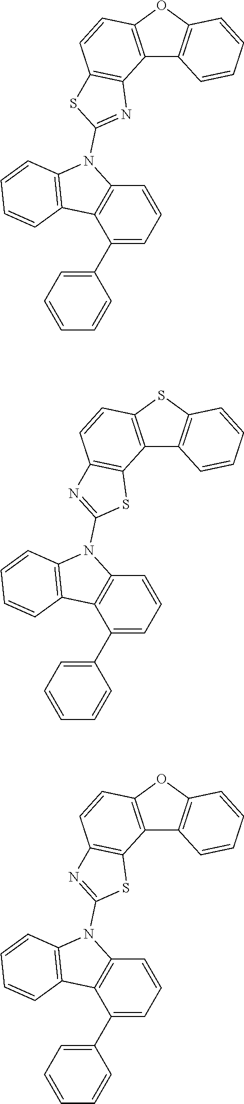 [SMILES: c1ccc(-c2cccc3c2c2ccccc2n3-c2nc3c(ccc4oc5ccccc5c43)s2)cc1 ; c1ccc(-c2cccc3c2c2ccccc2n3-c2nc3ccc4sc5ccccc5c4c3s2)cc1 ; c1ccc(-c2cccc3c2c2ccccc2n3-c2nc3ccc4oc5ccccc5c4c3s2)cc1]

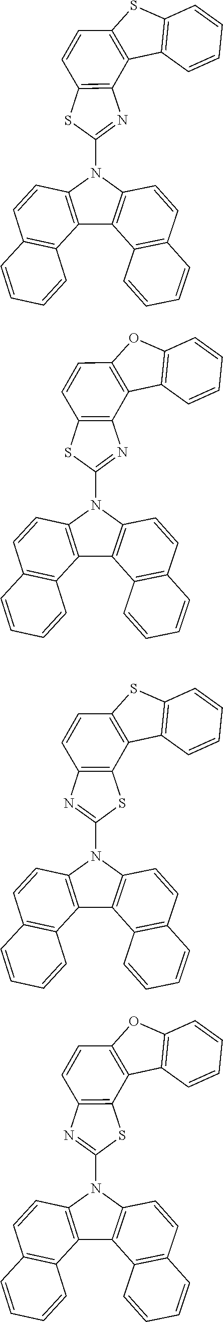

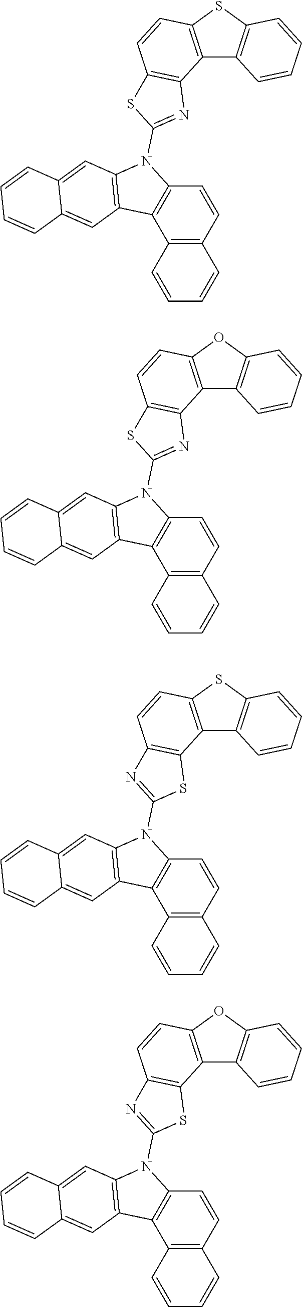

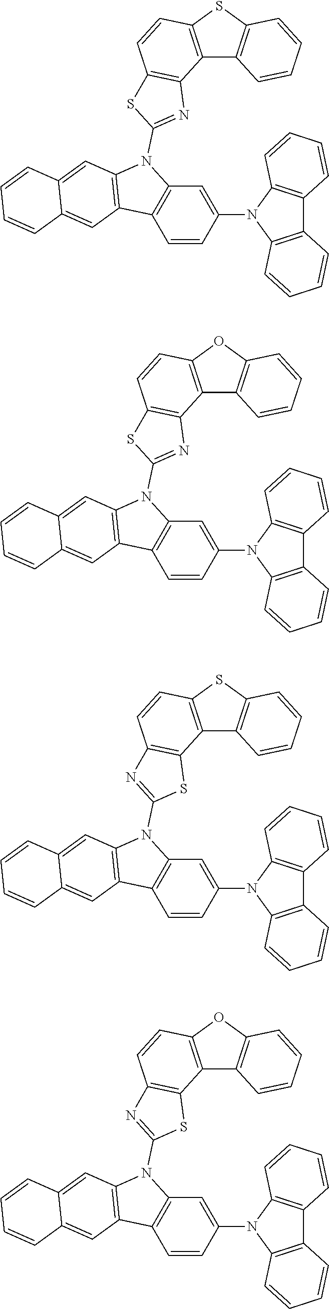 [SMILES: c1ccc2cc3c(cc2c1)c1ccc(-n2c4ccccc4c4ccccc42)cc1n3-c1nc2c(ccc3sc4ccccc4c32)s1 ; c1ccc2cc3c(cc2c1)c1ccc(-n2c4ccccc4c4ccccc42)cc1n3-c1nc2c(ccc3oc4ccccc4c32)s1 ; c1ccc2cc3c(cc2c1)c1ccc(-n2c4ccccc4c4ccccc42)cc1n3-c1nc2ccc3sc4ccccc4c3c2s1 ; c1ccc2cc3c(cc2c1)c1ccc(-n2c4ccccc4c4ccccc42)cc1n3-c1nc2ccc3oc4ccccc4c3c2s1]

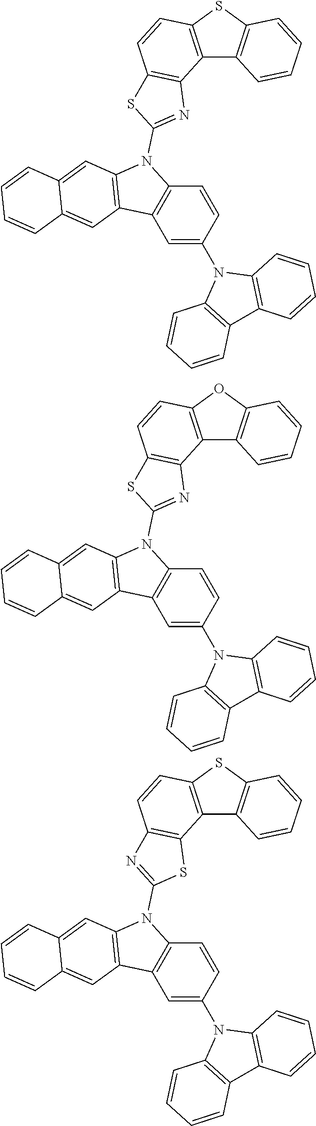

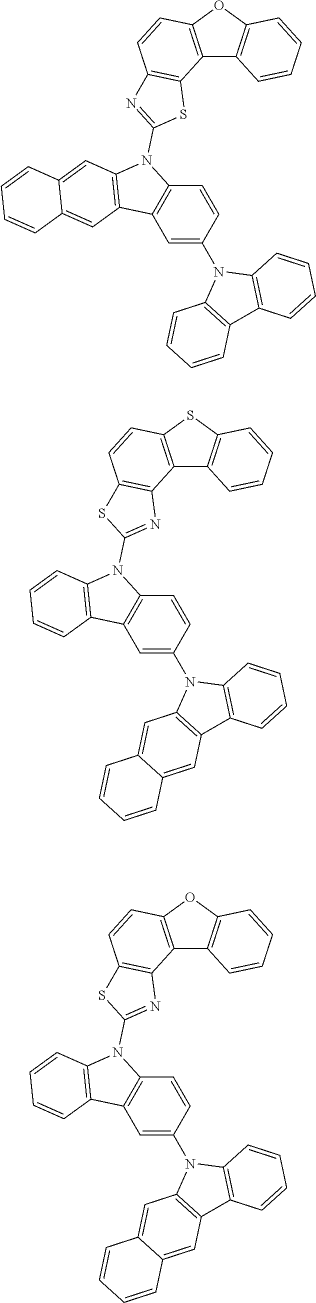 [SMILES: c1ccc2cc3c(cc2c1)c1cc(-n2c4ccccc4c4ccccc42)ccc1n3-c1nc2ccc3oc4ccccc4c3c2s1 ; c1ccc2cc3c(cc2c1)c1ccccc1n3-c1ccc2c(c1)c1ccccc1n2-c1nc2c(ccc3sc4ccccc4c32)s1 ; c1ccc2cc3c(cc2c1)c1ccccc1n3-c1ccc2c(c1)c1ccccc1n2-c1nc2c(ccc3oc4ccccc4c32)s1]

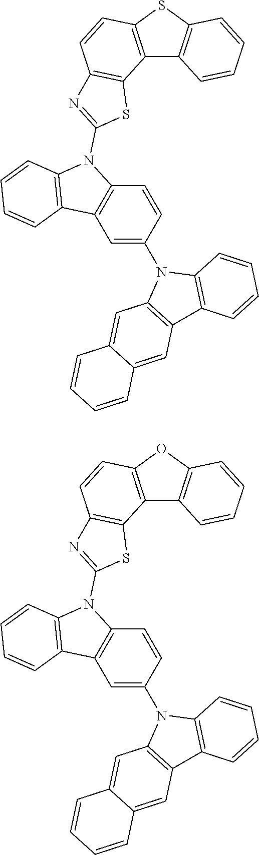 [SMILES: c1ccc2cc3c(cc2c1)c1ccccc1n3-c1ccc2c(c1)c1ccccc1n2-c1nc2ccc3sc4ccccc4c3c2s1 ; c1ccc2cc3c(cc2c1)c1ccccc1n3-c1ccc2c(c1)c1ccccc1n2-c1nc2ccc3oc4ccccc4c3c2s1]

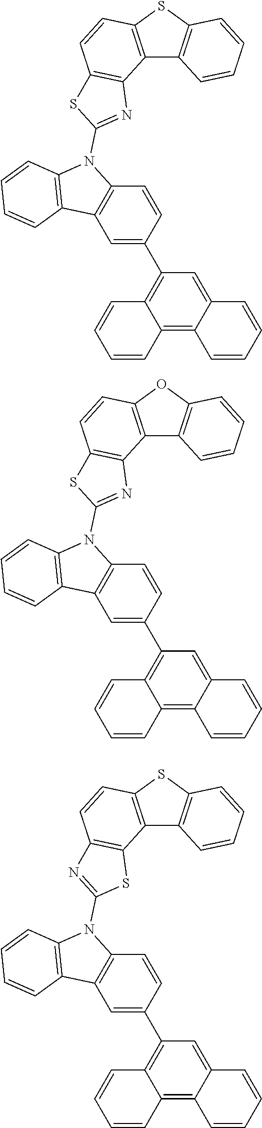

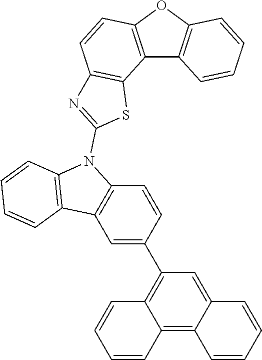 [SMILES: c1ccc2c(c1)cc(-c1ccc3c(c1)c1ccccc1n3-c1nc3ccc4oc5ccccc5c4c3s1)c1ccccc12]

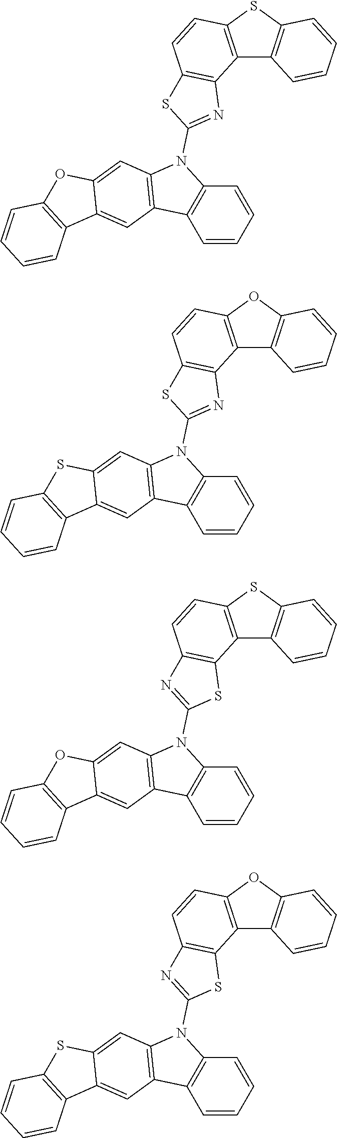 [SMILES: c1ccc2c(c1)oc1cc3c(cc12)c1ccccc1n3-c1nc2c(ccc3sc4ccccc4c32)s1 ; c1ccc2c(c1)oc1ccc3sc(-n4c5ccccc5c5cc6c(cc54)sc4ccccc46)nc3c12 ; c1ccc2c(c1)oc1cc3c(cc12)c1ccccc1n3-c1nc2ccc3sc4ccccc4c3c2s1 ; c1ccc2c(c1)oc1ccc3nc(-n4c5ccccc5c5cc6c(cc54)sc4ccccc46)sc3c12]

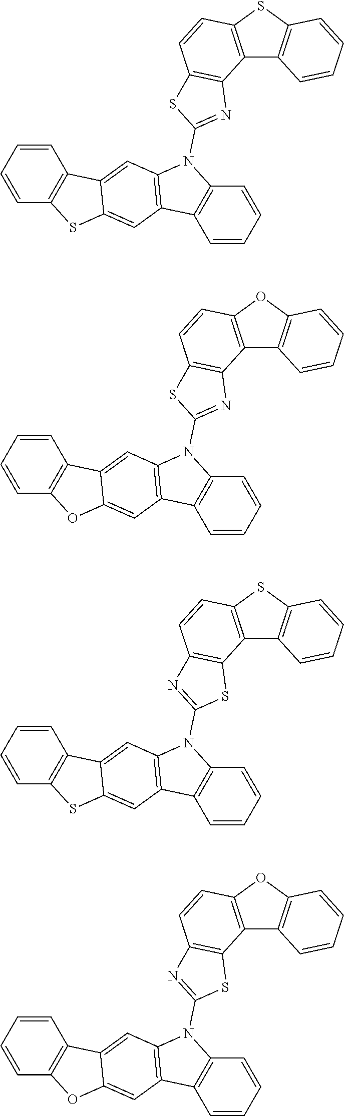 [SMILES: c1ccc2c(c1)sc1cc3c4ccccc4n(-c4nc5c(ccc6sc7ccccc7c65)s4)c3cc12 ; c1ccc2c(c1)oc1cc3c4ccccc4n(-c4nc5c(ccc6oc7ccccc7c65)s4)c3cc12 ; c1ccc2c(c1)sc1cc3c4ccccc4n(-c4nc5ccc6sc7ccccc7c6c5s4)c3cc12 ; c1ccc2c(c1)oc1cc3c4ccccc4n(-c4nc5ccc6oc7ccccc7c6c5s4)c3cc12]

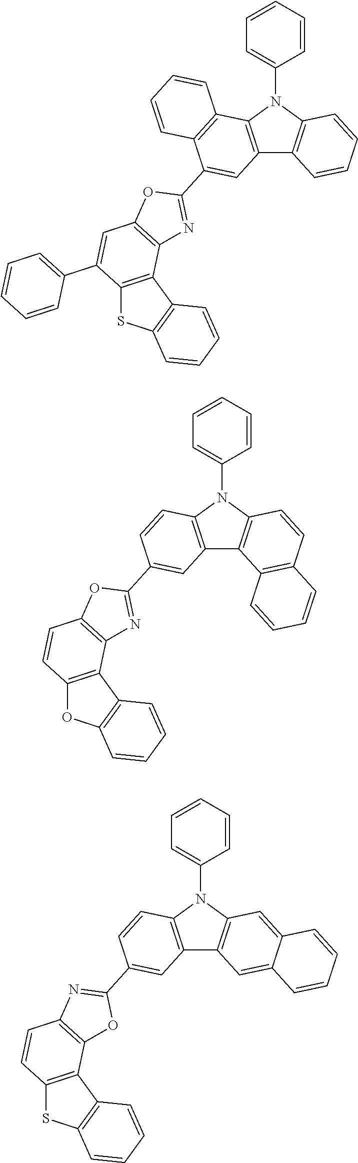

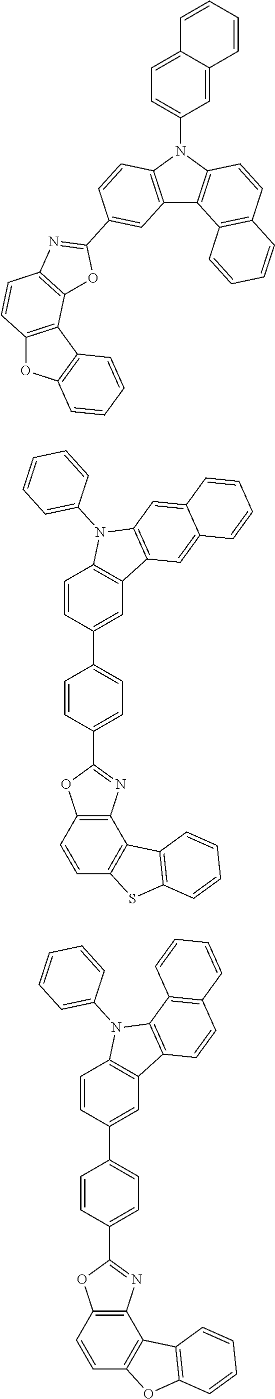

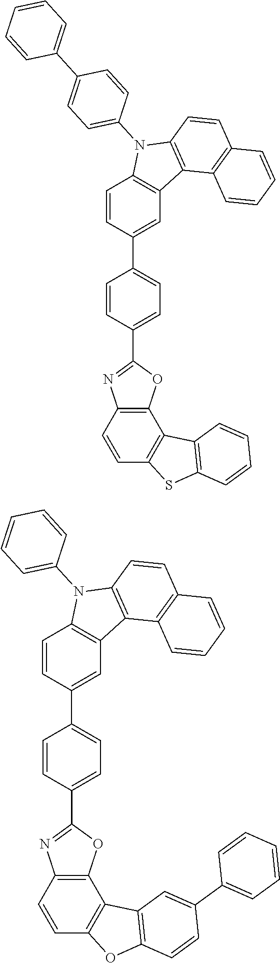 [SMILES: c1ccc(-c2ccc(-n3c4ccc(-c5ccc(-c6nc7ccc8sc9ccccc9c8c7o6)cc5)cc4c4c5ccccc5ccc43)cc2)cc1 ; c1ccc(-c2ccc3oc4ccc5nc(-c6ccc(-c7ccc8c(c7)c7c9ccccc9ccc7n8-c7ccccc7)cc6)oc5c4c3c2)cc1]

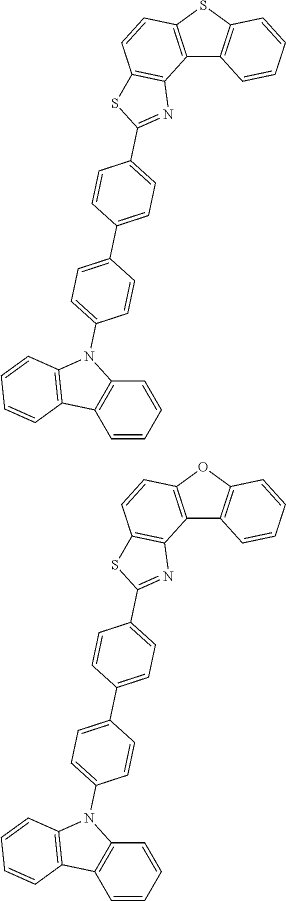

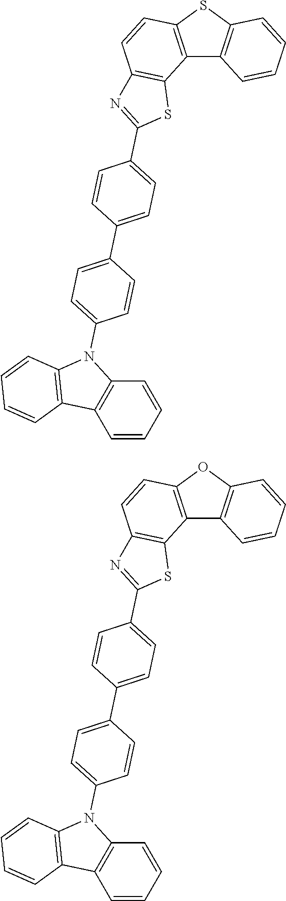

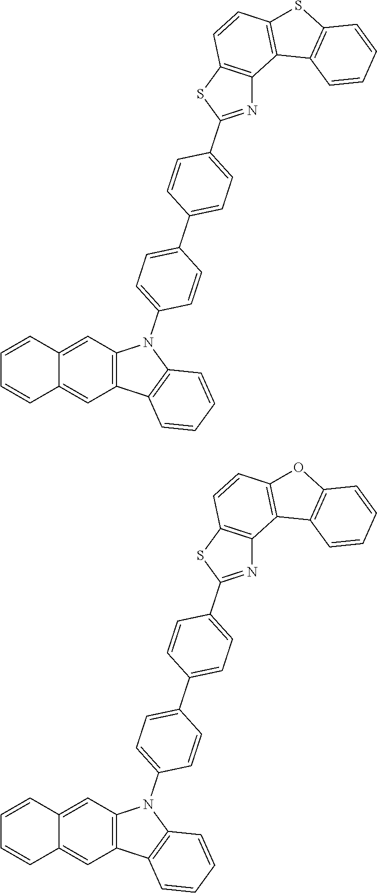 [SMILES: c1ccc2cc3c(cc2c1)c1ccccc1n3-c1ccc(-c2ccc(-c3nc4c(ccc5sc6ccccc6c54)s3)cc2)cc1 ; c1ccc2cc3c(cc2c1)c1ccccc1n3-c1ccc(-c2ccc(-c3nc4c(ccc5oc6ccccc6c54)s3)cc2)cc1]

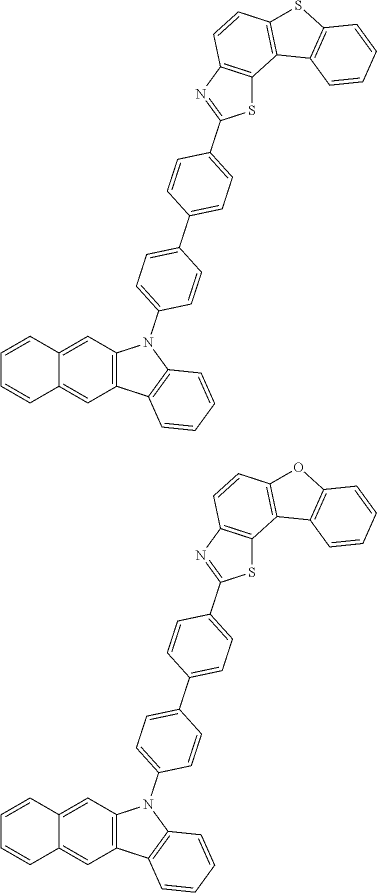 [SMILES: c1ccc2cc3c(cc2c1)c1ccccc1n3-c1ccc(-c2ccc(-c3nc4ccc5sc6ccccc6c5c4s3)cc2)cc1 ; c1ccc2cc3c(cc2c1)c1ccccc1n3-c1ccc(-c2ccc(-c3nc4ccc5oc6ccccc6c5c4s3)cc2)cc1]

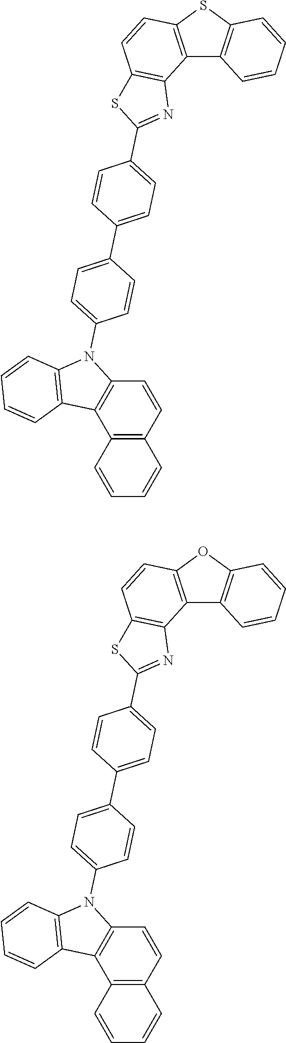 [SMILES: c1ccc2c(c1)ccc1c2c2ccccc2n1-c1ccc(-c2ccc(-c3nc4c(ccc5sc6ccccc6c54)s3)cc2)cc1 ; c1ccc2c(c1)ccc1c2c2ccccc2n1-c1ccc(-c2ccc(-c3nc4c(ccc5oc6ccccc6c54)s3)cc2)cc1]

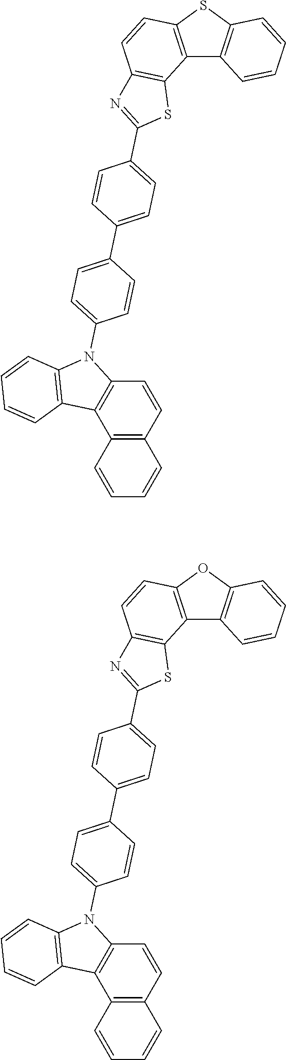 [SMILES: c1ccc2c(c1)ccc1c2c2ccccc2n1-c1ccc(-c2ccc(-c3nc4ccc5sc6ccccc6c5c4s3)cc2)cc1 ; c1ccc2c(c1)ccc1c2c2ccccc2n1-c1ccc(-c2ccc(-c3nc4ccc5oc6ccccc6c5c4s3)cc2)cc1]

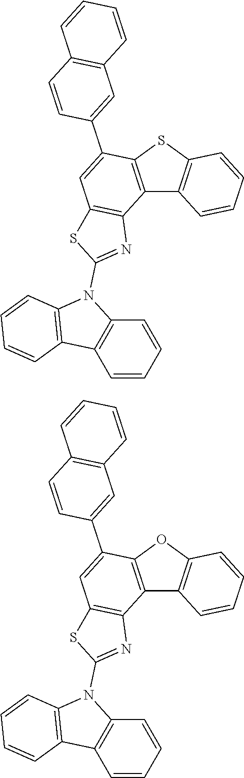

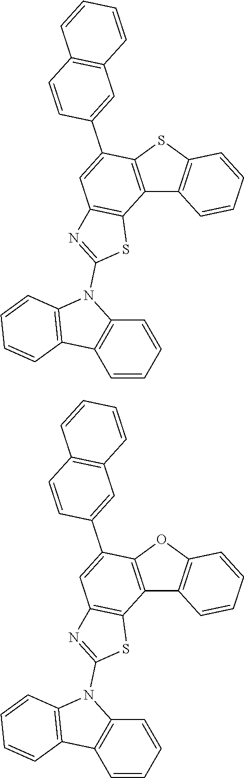

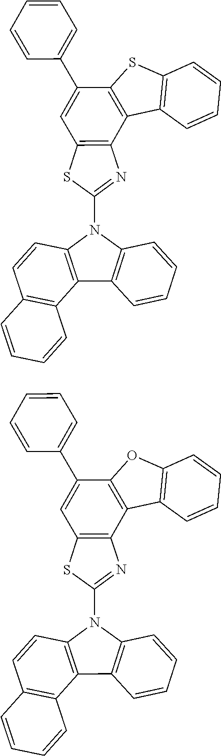

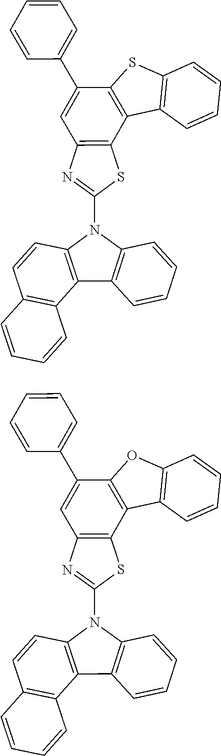

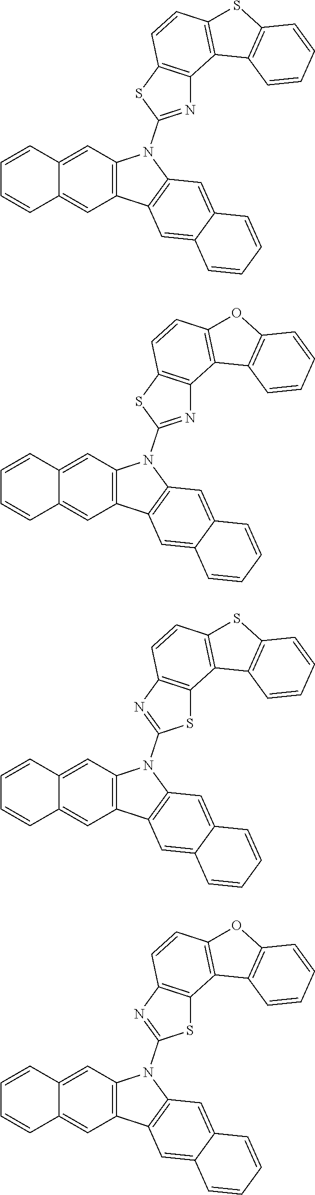

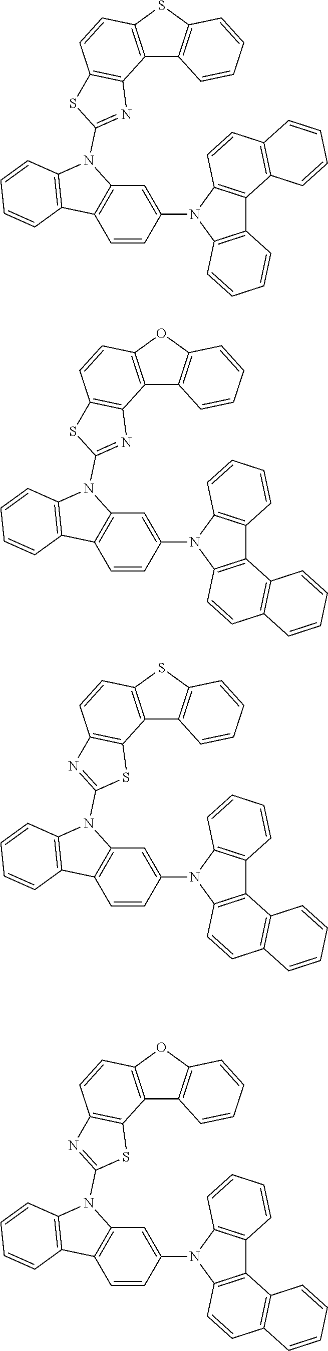 [SMILES: c1ccc2c(c1)ccc1c2c2ccccc2n1-c1ccc2c3ccccc3n(-c3nc4c(ccc5sc6ccccc6c54)s3)c2c1 ; c1ccc2c(c1)ccc1c2c2ccccc2n1-c1ccc2c3ccccc3n(-c3nc4c(ccc5oc6ccccc6c54)s3)c2c1 ; c1ccc2c(c1)ccc1c2c2ccccc2n1-c1ccc2c3ccccc3n(-c3nc4ccc5sc6ccccc6c5c4s3)c2c1 ; c1ccc2c(c1)ccc1c2c2ccccc2n1-c1ccc2c3ccccc3n(-c3nc4ccc5oc6ccccc6c5c4s3)c2c1]

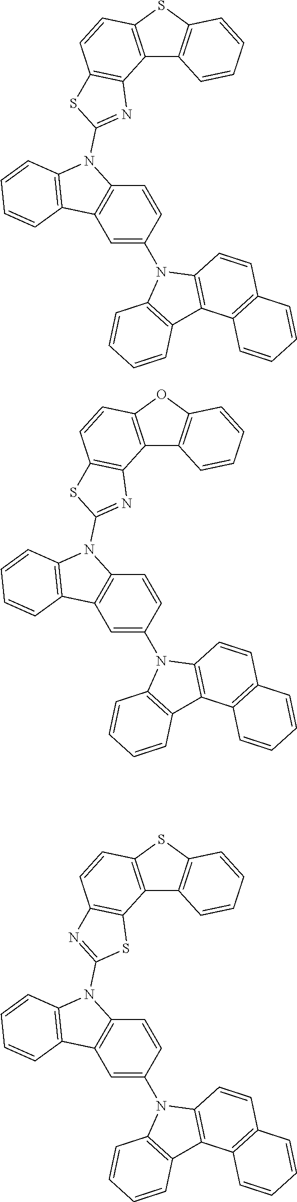

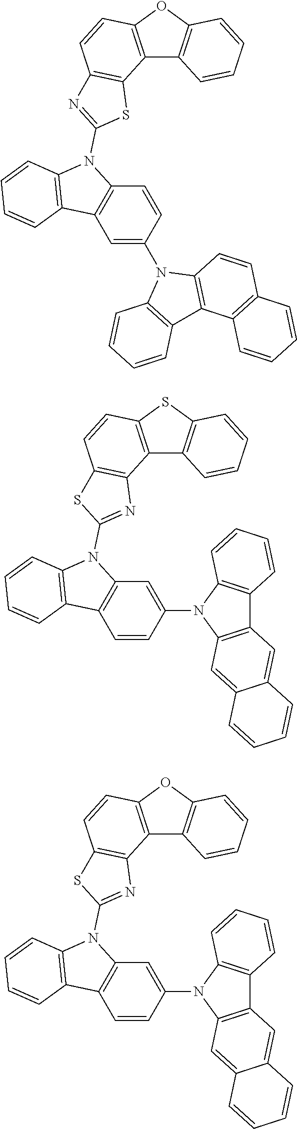

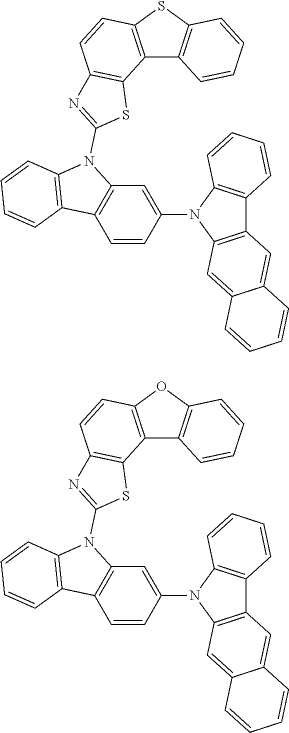 [SMILES: c1ccc2cc3c(cc2c1)c1ccccc1n3-c1ccc2c3ccccc3n(-c3nc4ccc5sc6ccccc6c5c4s3)c2c1 ; c1ccc2cc3c(cc2c1)c1ccccc1n3-c1ccc2c3ccccc3n(-c3nc4ccc5oc6ccccc6c5c4s3)c2c1]

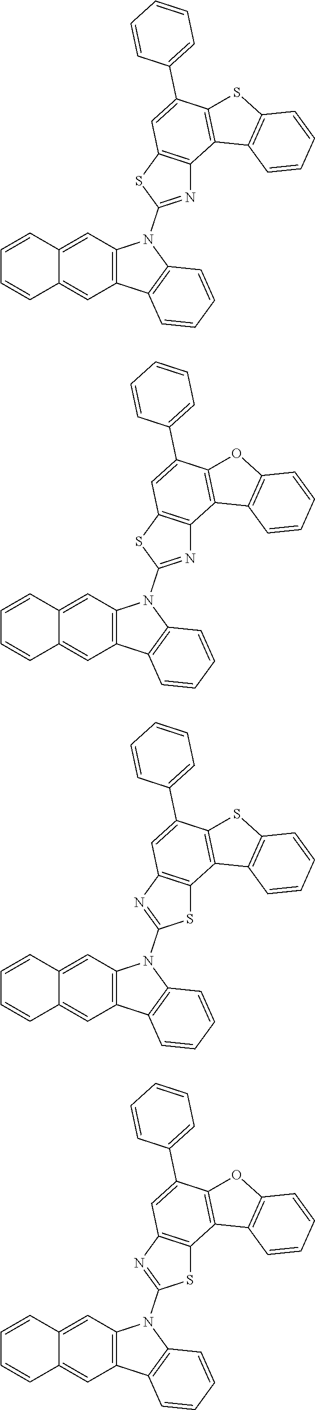

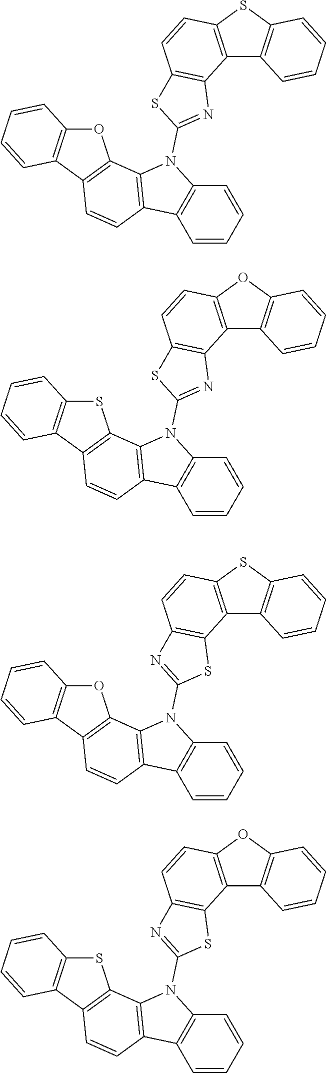

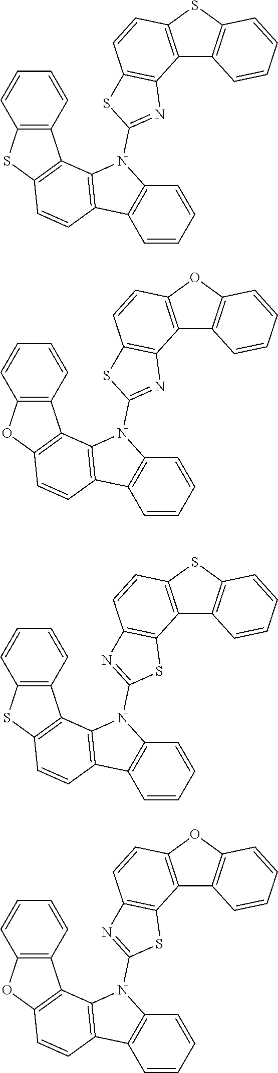

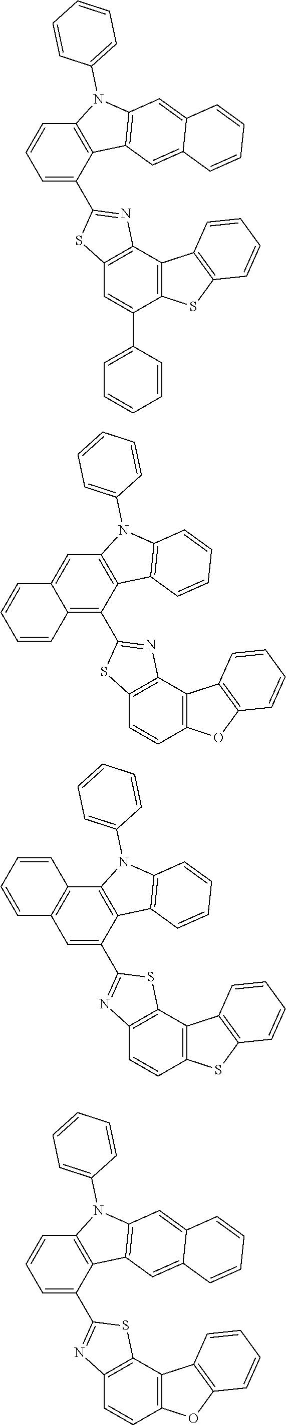

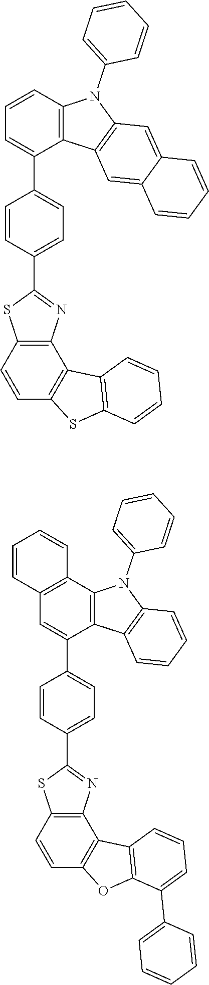 [SMILES: c1ccc(-n2c3cc4ccccc4cc3c3c(-c4ccc(-c5nc6c(ccc7sc8ccccc8c76)s5)cc4)cccc32)cc1 ; c1ccc(-c2cccc3c2oc2ccc4sc(-c5ccc(-c6cc7ccccc7c7c6c6ccccc6n7-c6ccccc6)cc5)nc4c23)cc1]

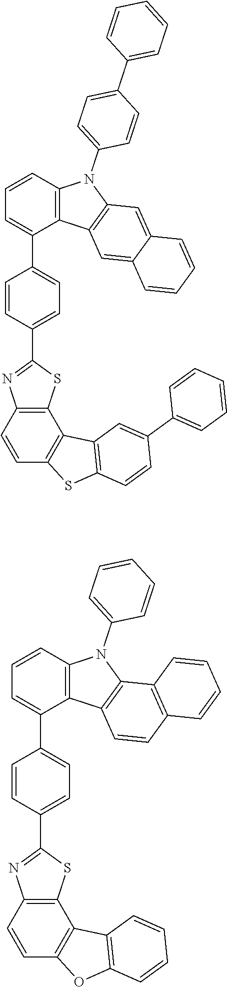 [SMILES: c1ccc(-c2ccc(-n3c4cc5ccccc5cc4c4c(-c5ccc(-c6nc7ccc8sc9ccc(-c%10ccccc%10)cc9c8c7s6)cc5)cccc43)cc2)cc1 ; c1ccc(-n2c3cccc(-c4ccc(-c5nc6ccc7oc8ccccc8c7c6s5)cc4)c3c3ccc4ccccc4c32)cc1]

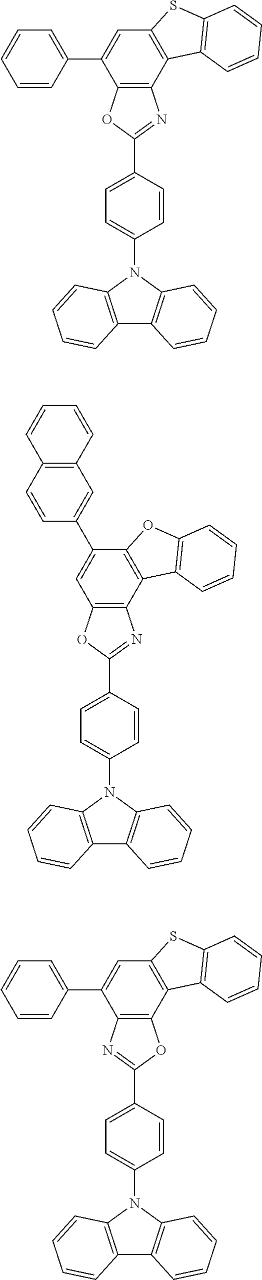 [SMILES: c1ccc(-c2cc3sc4ccccc4c3c3nc(-c4ccc(-n5c6ccccc6c6ccccc65)cc4)oc23)cc1 ; c1ccc2cc(-c3cc4oc(-c5ccc(-n6c7ccccc7c7ccccc76)cc5)nc4c4c3oc3ccccc34)ccc2c1 ; c1ccc(-c2cc3sc4ccccc4c3c3oc(-c4ccc(-n5c6ccccc6c6ccccc65)cc4)nc23)cc1]

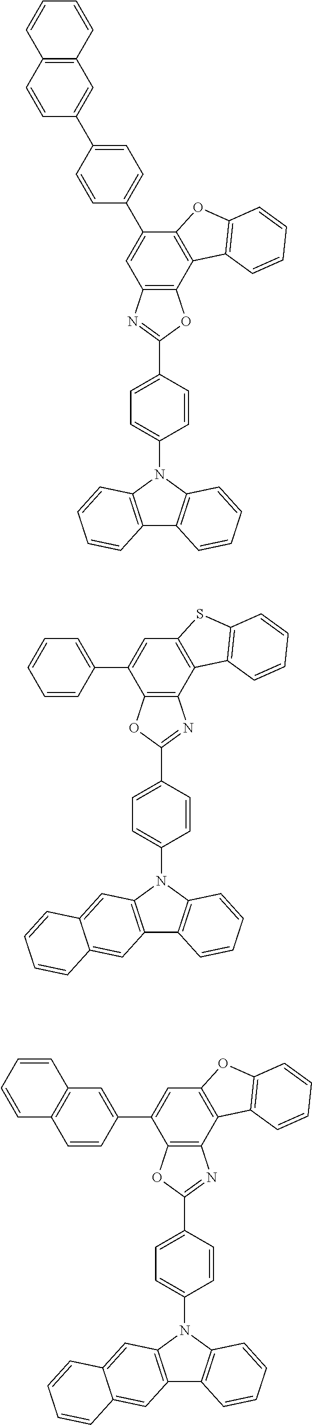 [SMILES: c1ccc2cc(-c3ccc(-c4cc5nc(-c6ccc(-n7c8ccccc8c8ccccc87)cc6)oc5c5c4oc4ccccc45)cc3)ccc2c1 ; c1ccc(-c2cc3sc4ccccc4c3c3nc(-c4ccc(-n5c6ccccc6c6cc7ccccc7cc65)cc4)oc23)cc1 ; c1ccc2cc(-c3cc4oc5ccccc5c4c4nc(-c5ccc(-n6c7ccccc7c7cc8ccccc8cc76)cc5)oc34)ccc2c1]

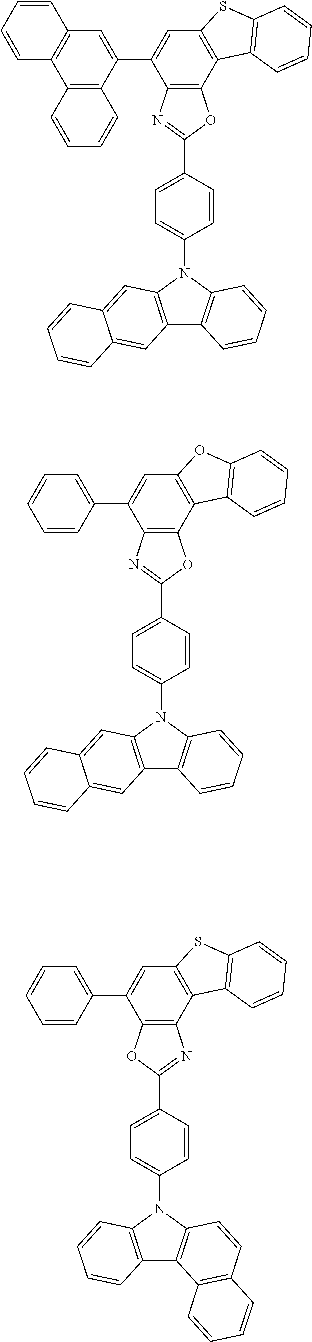 [SMILES: c1ccc2cc3c(cc2c1)c1ccccc1n3-c1ccc(-c2nc3c(-c4cc5ccccc5c5ccccc45)cc4sc5ccccc5c4c3o2)cc1 ; c1ccc(-c2cc3oc4ccccc4c3c3oc(-c4ccc(-n5c6ccccc6c6cc7ccccc7cc65)cc4)nc23)cc1 ; c1ccc(-c2cc3sc4ccccc4c3c3nc(-c4ccc(-n5c6ccccc6c6c7ccccc7ccc65)cc4)oc23)cc1]

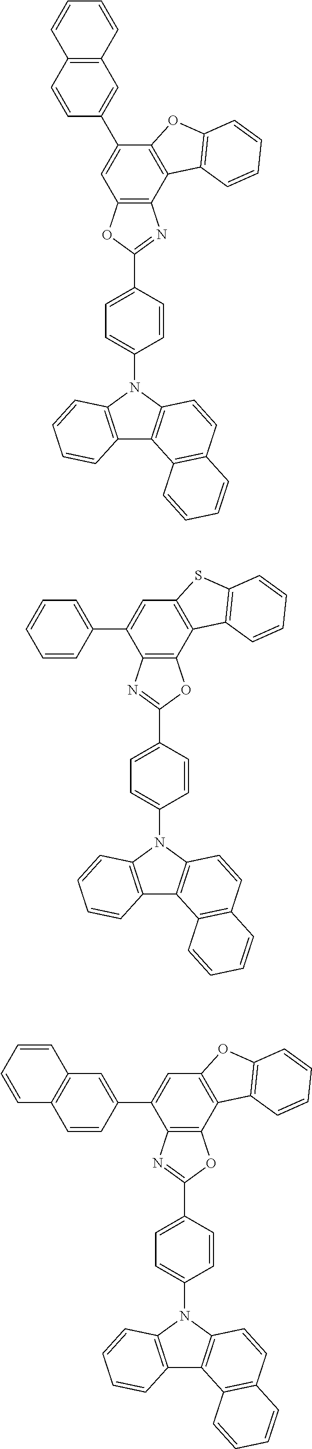 [SMILES: c1ccc2cc(-c3cc4oc(-c5ccc(-n6c7ccccc7c7c8ccccc8ccc76)cc5)nc4c4c3oc3ccccc34)ccc2c1 ; c1ccc(-c2cc3sc4ccccc4c3c3oc(-c4ccc(-n5c6ccccc6c6c7ccccc7ccc65)cc4)nc23)cc1 ; c1ccc2cc(-c3cc4oc5ccccc5c4c4oc(-c5ccc(-n6c7ccccc7c7c8ccccc8ccc76)cc5)nc34)ccc2c1]

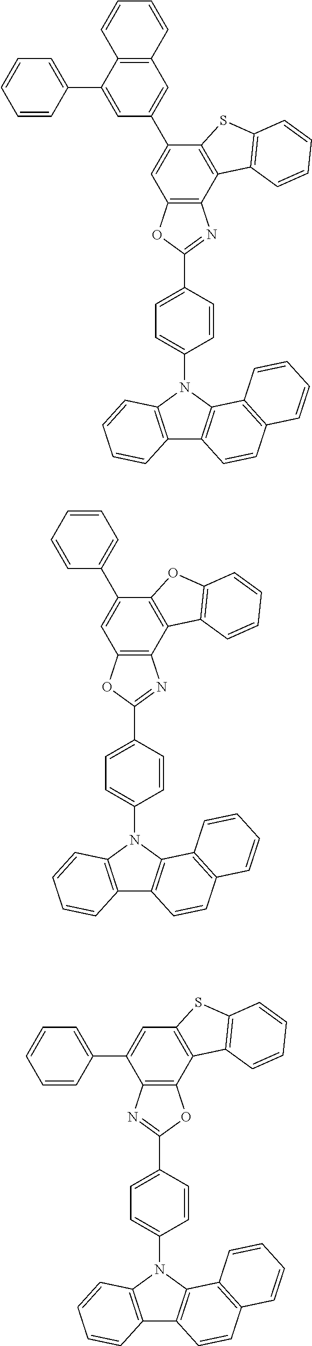 [SMILES: c1ccc(-c2cc(-c3cc4oc(-c5ccc(-n6c7ccccc7c7ccc8ccccc8c76)cc5)nc4c4c3sc3ccccc34)cc3ccccc23)cc1 ; c1ccc(-c2cc3oc(-c4ccc(-n5c6ccccc6c6ccc7ccccc7c65)cc4)nc3c3c2oc2ccccc23)cc1 ; c1ccc(-c2cc3sc4ccccc4c3c3oc(-c4ccc(-n5c6ccccc6c6ccc7ccccc7c65)cc4)nc23)cc1]

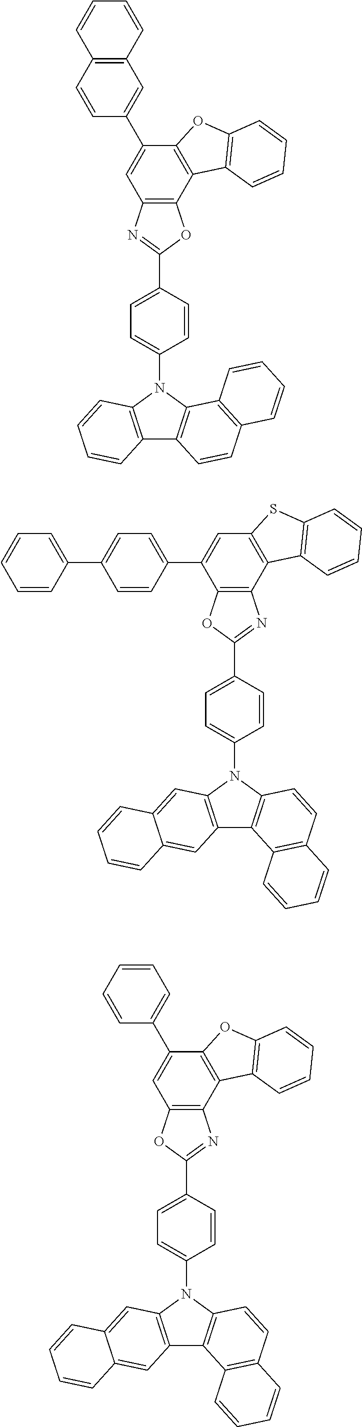 [SMILES: c1ccc2cc(-c3cc4nc(-c5ccc(-n6c7ccccc7c7ccc8ccccc8c76)cc5)oc4c4c3oc3ccccc34)ccc2c1 ; c1ccc(-c2ccc(-c3cc4sc5ccccc5c4c4nc(-c5ccc(-n6c7cc8ccccc8cc7c7c8ccccc8ccc76)cc5)oc34)cc2)cc1 ; c1ccc(-c2cc3oc(-c4ccc(-n5c6cc7ccccc7cc6c6c7ccccc7ccc65)cc4)nc3c3c2oc2ccccc23)cc1]

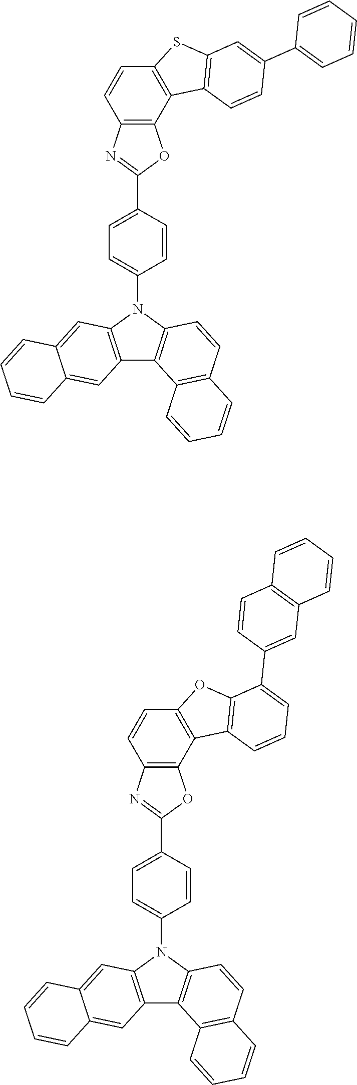 [SMILES: c1ccc(-c2ccc3c(c2)sc2ccc4nc(-c5ccc(-n6c7cc8ccccc8cc7c7c8ccccc8ccc76)cc5)oc4c23)cc1 ; c1ccc2cc(-c3cccc4c3oc3ccc5nc(-c6ccc(-n7c8cc9ccccc9cc8c8c9ccccc9ccc87)cc6)oc5c34)ccc2c1]

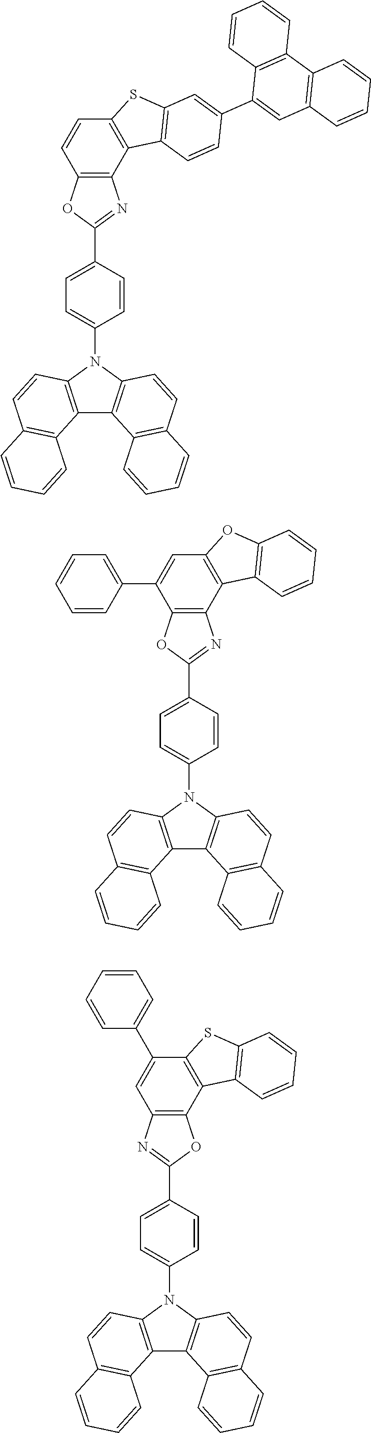

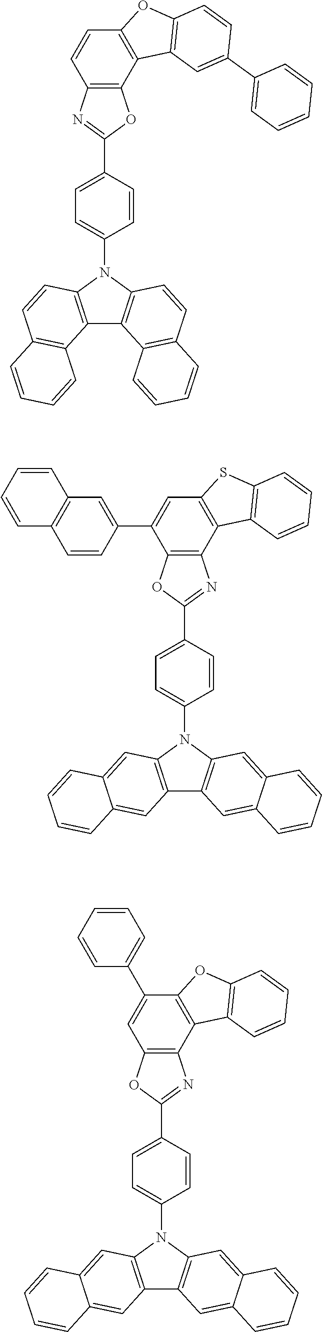 [SMILES: c1ccc(-c2ccc3oc4ccc5nc(-c6ccc(-n7c8ccc9ccccc9c8c8c9ccccc9ccc87)cc6)oc5c4c3c2)cc1 ; c1ccc2cc(-c3cc4sc5ccccc5c4c4nc(-c5ccc(-n6c7cc8ccccc8cc7c7cc8ccccc8cc76)cc5)oc34)ccc2c1 ; c1ccc(-c2cc3oc(-c4ccc(-n5c6cc7ccccc7cc6c6cc7ccccc7cc65)cc4)nc3c3c2oc2ccccc23)cc1]

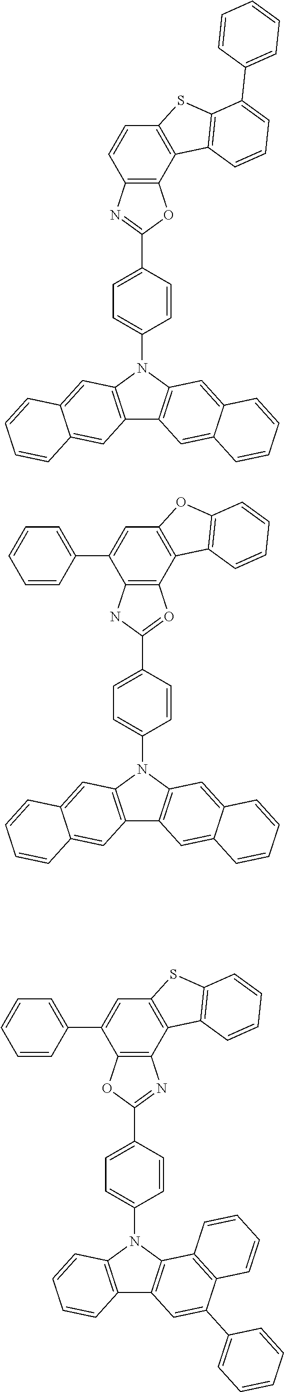

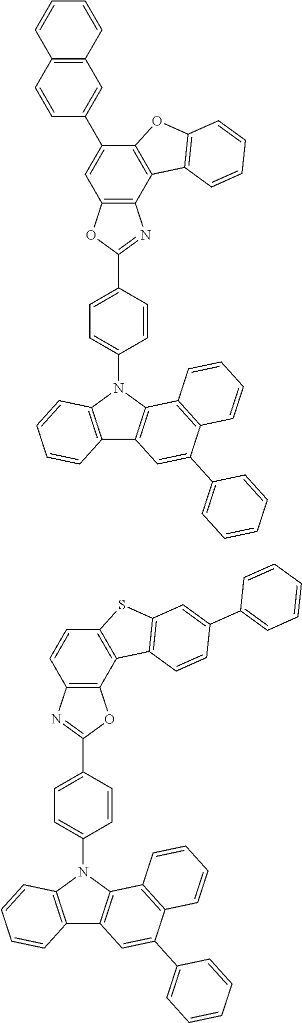

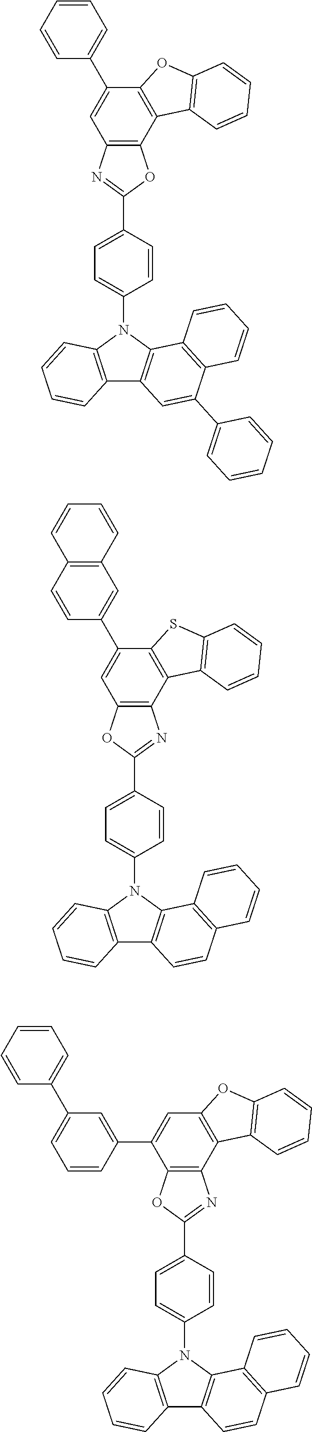 [SMILES: c1ccc(-c2cc3c4ccccc4n(-c4ccc(-c5nc6cc(-c7ccccc7)c7oc8ccccc8c7c6o5)cc4)c3c3ccccc23)cc1 ; c1ccc2cc(-c3cc4oc(-c5ccc(-n6c7ccccc7c7ccc8ccccc8c76)cc5)nc4c4c3sc3ccccc34)ccc2c1 ; c1ccc(-c2cccc(-c3cc4oc5ccccc5c4c4nc(-c5ccc(-n6c7ccccc7c7ccc8ccccc8c76)cc5)oc34)c2)cc1]

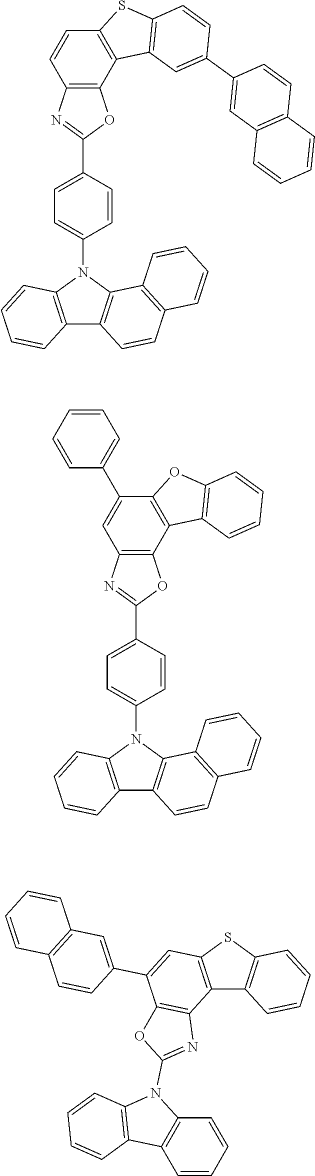 [SMILES: c1ccc2cc(-c3ccc4sc5ccc6nc(-c7ccc(-n8c9ccccc9c9ccc%10ccccc%10c98)cc7)oc6c5c4c3)ccc2c1 ; c1ccc(-c2cc3nc(-c4ccc(-n5c6ccccc6c6ccc7ccccc7c65)cc4)oc3c3c2oc2ccccc23)cc1 ; c1ccc2cc(-c3cc4sc5ccccc5c4c4nc(-n5c6ccccc6c6ccccc65)oc34)ccc2c1]

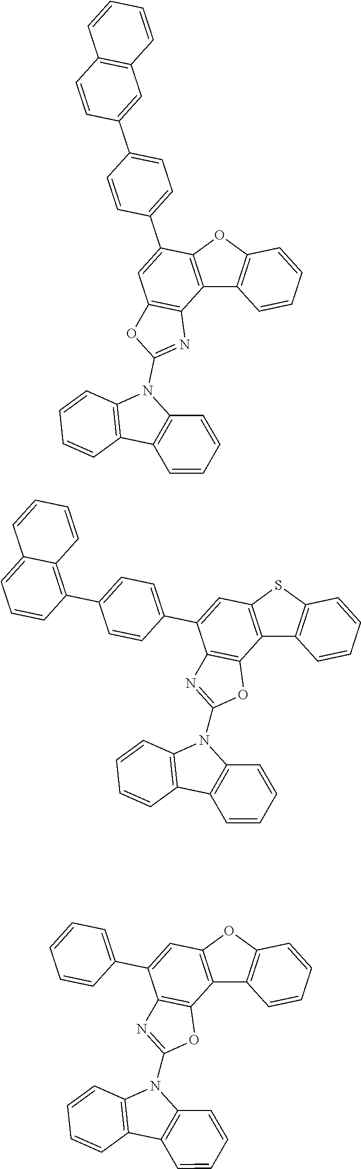 [SMILES: c1ccc2cc(-c3ccc(-c4cc5oc(-n6c7ccccc7c7ccccc76)nc5c5c4oc4ccccc45)cc3)ccc2c1 ; c1ccc2c(-c3ccc(-c4cc5sc6ccccc6c5c5oc(-n6c7ccccc7c7ccccc76)nc45)cc3)cccc2c1 ; c1ccc(-c2cc3oc4ccccc4c3c3oc(-n4c5ccccc5c5ccccc54)nc23)cc1]

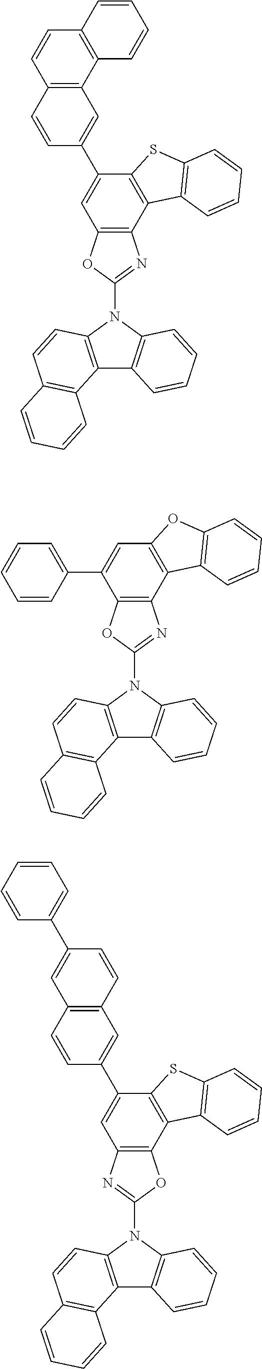 [SMILES: c1ccc2c(c1)ccc1ccc(-c3cc4oc(-n5c6ccccc6c6c7ccccc7ccc65)nc4c4c3sc3ccccc34)cc12 ; c1ccc(-c2cc3oc4ccccc4c3c3nc(-n4c5ccccc5c5c6ccccc6ccc54)oc23)cc1 ; c1ccc(-c2ccc3cc(-c4cc5nc(-n6c7ccccc7c7c8ccccc8ccc76)oc5c5c4sc4ccccc45)ccc3c2)cc1]

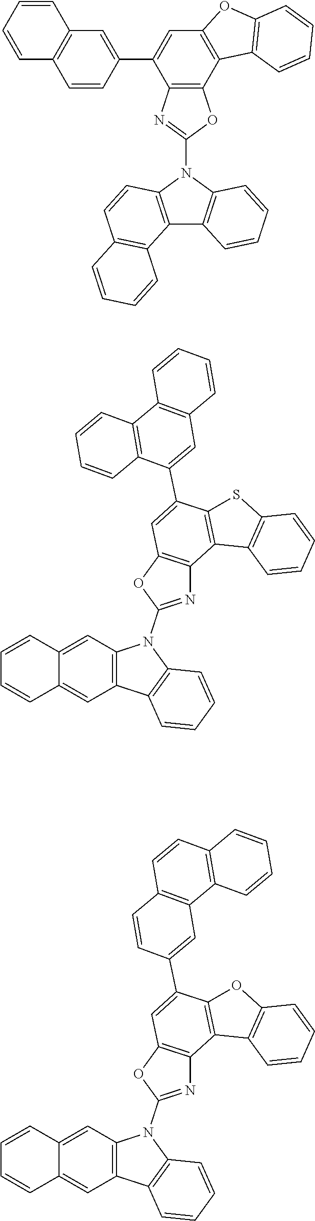 [SMILES: c1ccc2cc(-c3cc4oc5ccccc5c4c4oc(-n5c6ccccc6c6c7ccccc7ccc65)nc34)ccc2c1 ; c1ccc2cc3c(cc2c1)c1ccccc1n3-c1nc2c(cc(-c3cc4ccccc4c4ccccc34)c3sc4ccccc4c32)o1 ; c1ccc2cc3c(cc2c1)c1ccccc1n3-c1nc2c(cc(-c3ccc4ccc5ccccc5c4c3)c3oc4ccccc4c32)o1]

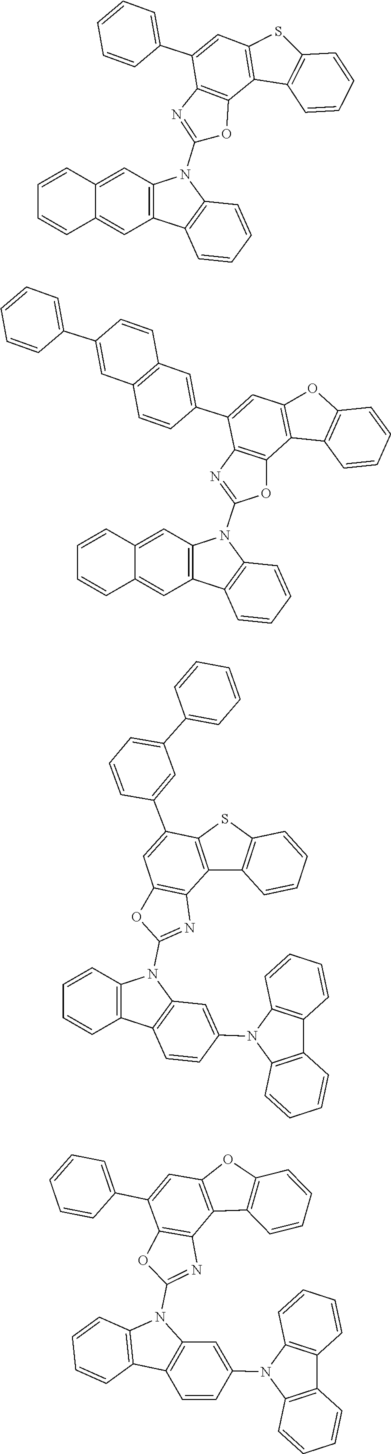 [SMILES: c1ccc(-c2cc3sc4ccccc4c3c3oc(-n4c5ccccc5c5cc6ccccc6cc54)nc23)cc1 ; c1ccc(-c2ccc3cc(-c4cc5oc6ccccc6c5c5oc(-n6c7ccccc7c7cc8ccccc8cc76)nc45)ccc3c2)cc1 ; c1ccc(-c2cccc(-c3cc4oc(-n5c6ccccc6c6ccc(-n7c8ccccc8c8ccccc87)cc65)nc4c4c3sc3ccccc34)c2)cc1 ; c1ccc(-c2cc3oc4ccccc4c3c3nc(-n4c5ccccc5c5ccc(-n6c7ccccc7c7ccccc76)cc54)oc23)cc1]

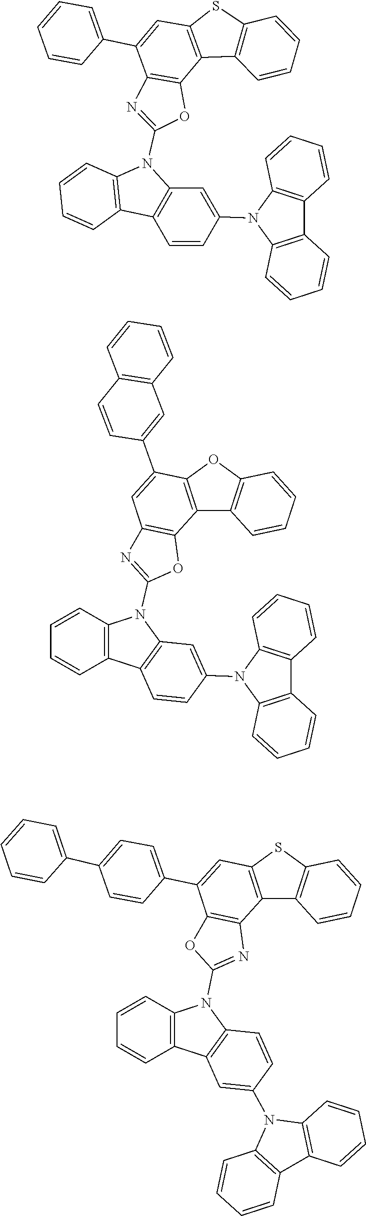 [SMILES: c1ccc(-c2cc3sc4ccccc4c3c3oc(-n4c5ccccc5c5ccc(-n6c7ccccc7c7ccccc76)cc54)nc23)cc1 ; c1ccc2cc(-c3cc4nc(-n5c6ccccc6c6ccc(-n7c8ccccc8c8ccccc87)cc65)oc4c4c3oc3ccccc34)ccc2c1 ; c1ccc(-c2ccc(-c3cc4sc5ccccc5c4c4nc(-n5c6ccccc6c6cc(-n7c8ccccc8c8ccccc87)ccc65)oc34)cc2)cc1]

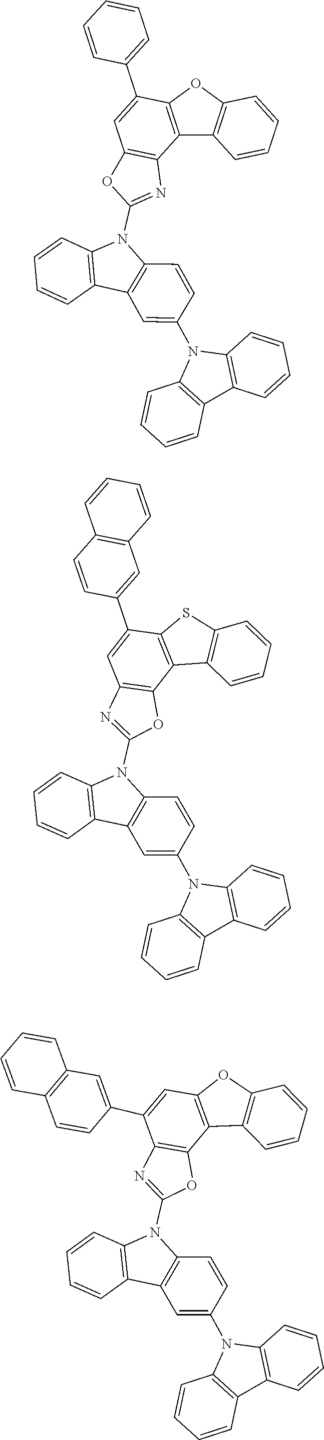

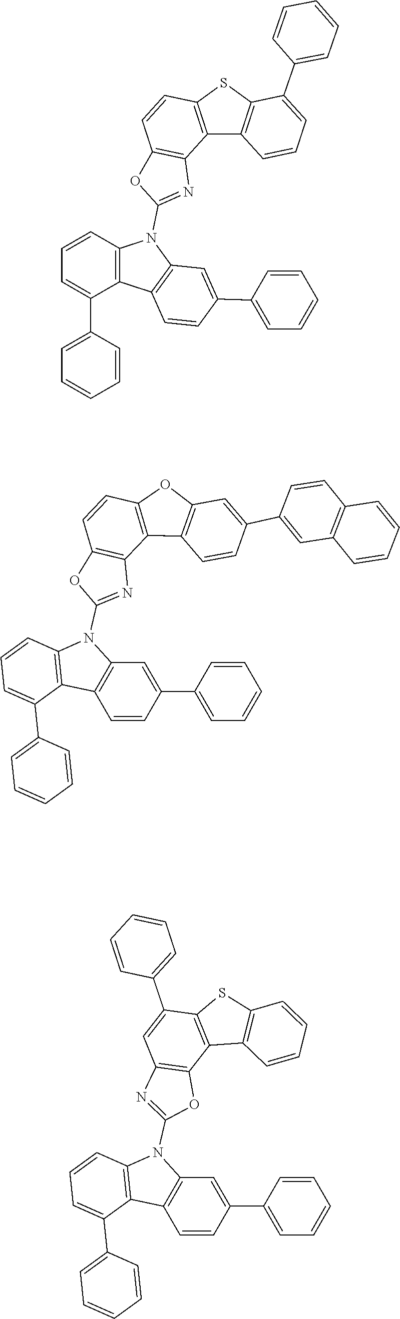 [SMILES: c1ccc(-c2ccc3c4c(-c5ccccc5)cccc4n(-c4nc5c(ccc6sc7c(-c8ccccc8)cccc7c65)o4)c3c2)cc1 ; c1ccc(-c2ccc3c4c(-c5ccccc5)cccc4n(-c4nc5c(ccc6oc7cc(-c8ccc9ccccc9c8)ccc7c65)o4)c3c2)cc1 ; c1ccc(-c2ccc3c4c(-c5ccccc5)cccc4n(-c4nc5cc(-c6ccccc6)c6sc7ccccc7c6c5o4)c3c2)cc1]

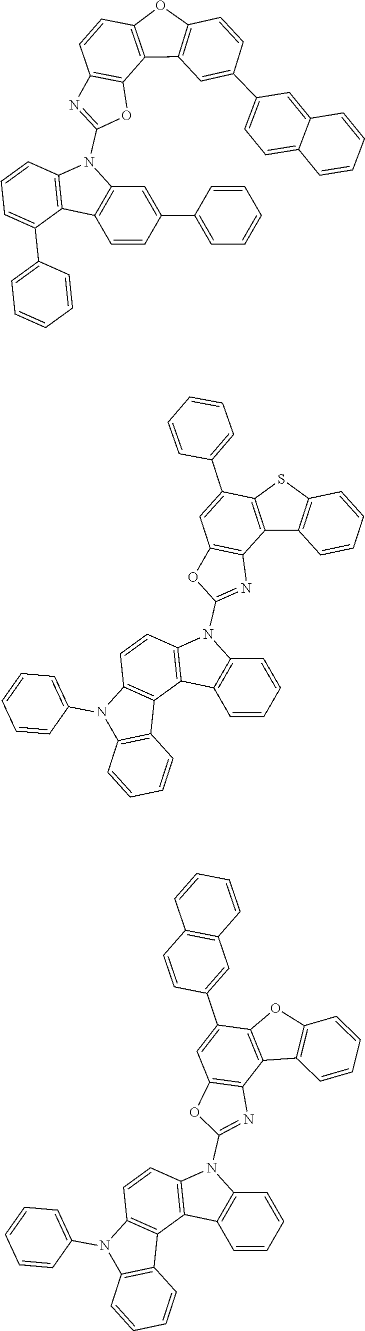 [SMILES: c1ccc(-c2ccc3c4c(-c5ccccc5)cccc4n(-c4nc5ccc6oc7ccc(-c8ccc9ccccc9c8)cc7c6c5o4)c3c2)cc1 ; c1ccc(-c2cc3oc(-n4c5ccccc5c5c6c7ccccc7n(-c7ccccc7)c6ccc54)nc3c3c2sc2ccccc23)cc1 ; c1ccc(-n2c3ccccc3c3c4c5ccccc5n(-c5nc6c(cc(-c7ccc8ccccc8c7)c7oc8ccccc8c76)o5)c4ccc32)cc1]

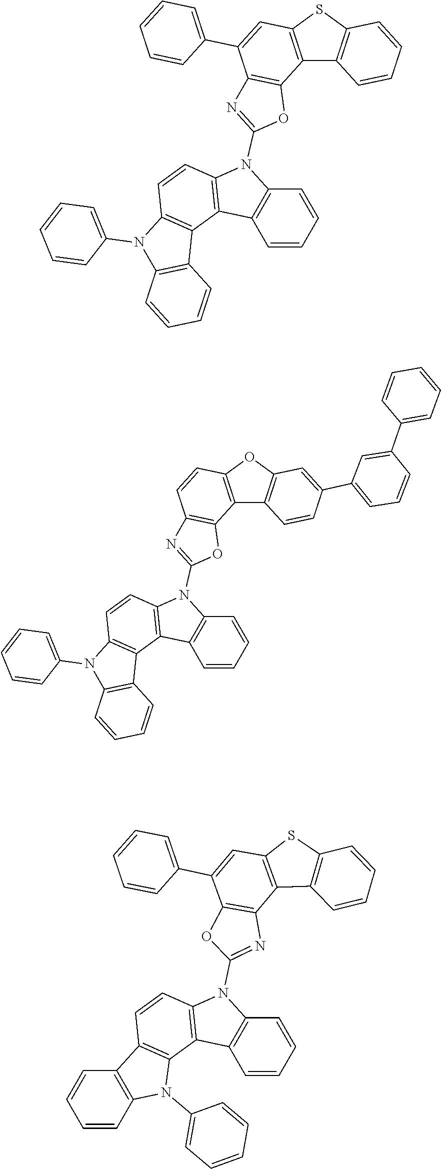 [SMILES: c1ccc(-c2cc3sc4ccccc4c3c3oc(-n4c5ccccc5c5c6c7ccccc7n(-c7ccccc7)c6ccc54)nc23)cc1 ; c1ccc(-c2cccc(-c3ccc4c(c3)oc3ccc5nc(-n6c7ccccc7c7c8c9ccccc9n(-c9ccccc9)c8ccc76)oc5c34)c2)cc1 ; c1ccc(-c2cc3sc4ccccc4c3c3nc(-n4c5ccccc5c5c4ccc4c6ccccc6n(-c6ccccc6)c45)oc23)cc1]

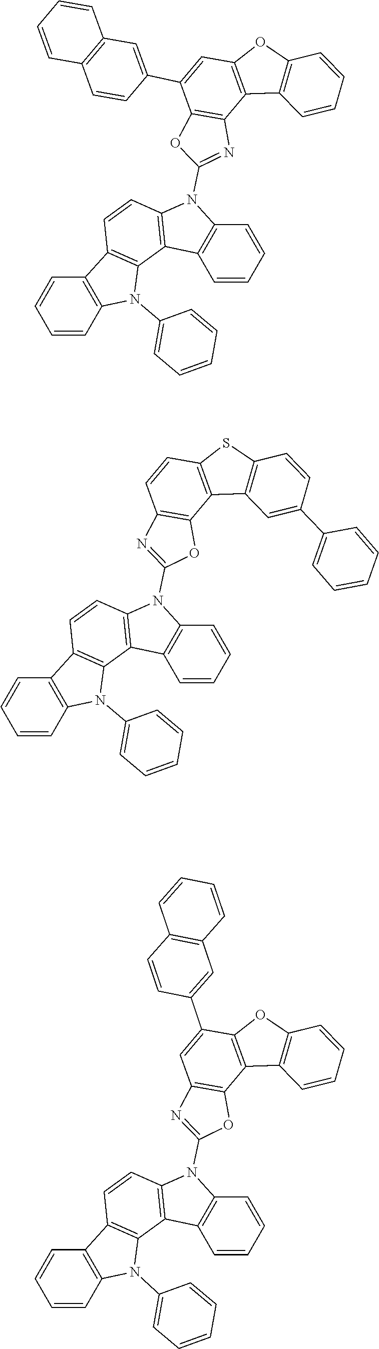 [SMILES: c1ccc(-n2c3ccccc3c3ccc4c(c5ccccc5n4-c4nc5c(o4)c(-c4ccc6ccccc6c4)cc4oc6ccccc6c45)c32)cc1 ; c1ccc(-c2ccc3sc4ccc5nc(-n6c7ccccc7c7c6ccc6c8ccccc8n(-c8ccccc8)c67)oc5c4c3c2)cc1 ; c1ccc(-n2c3ccccc3c3ccc4c(c5ccccc5n4-c4nc5cc(-c6ccc7ccccc7c6)c6oc7ccccc7c6c5o4)c32)cc1]

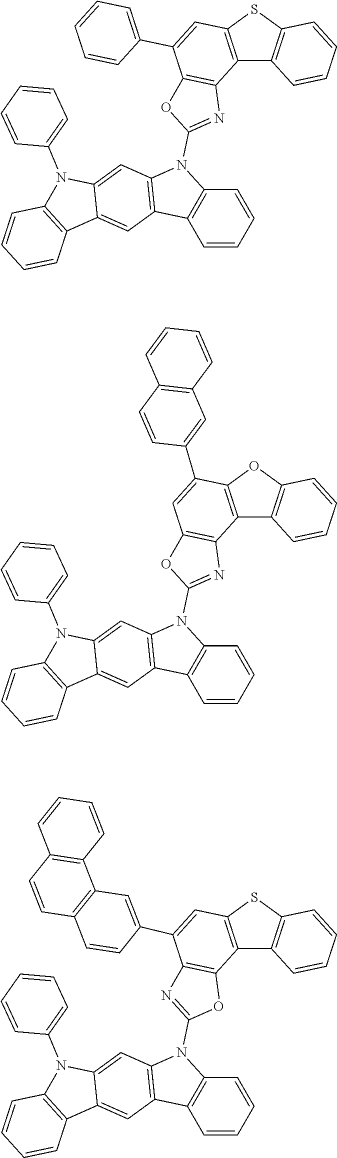 [SMILES: c1ccc(-c2cc3sc4ccccc4c3c3nc(-n4c5ccccc5c5cc6c7ccccc7n(-c7ccccc7)c6cc54)oc23)cc1 ; c1ccc(-n2c3ccccc3c3cc4c5ccccc5n(-c5nc6c(cc(-c7ccc8ccccc8c7)c7oc8ccccc8c76)o5)c4cc32)cc1 ; c1ccc(-n2c3ccccc3c3cc4c5ccccc5n(-c5nc6c(-c7ccc8ccc9ccccc9c8c7)cc7sc8ccccc8c7c6o5)c4cc32)cc1]

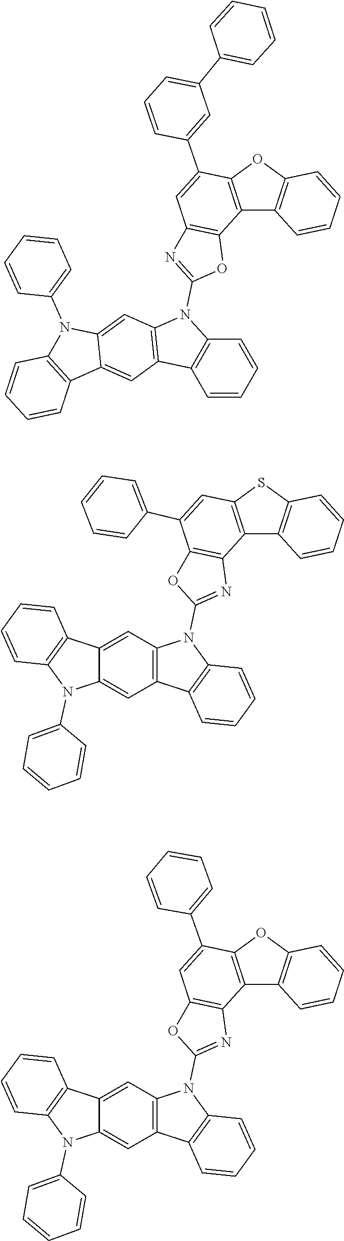 [SMILES: c1ccc(-c2cccc(-c3cc4nc(-n5c6ccccc6c6cc7c8ccccc8n(-c8ccccc8)c7cc65)oc4c4c3oc3ccccc34)c2)cc1 ; c1ccc(-c2cc3sc4ccccc4c3c3nc(-n4c5ccccc5c5cc6c(cc54)c4ccccc4n6-c4ccccc4)oc23)cc1 ; c1ccc(-c2cc3oc(-n4c5ccccc5c5cc6c(cc54)c4ccccc4n6-c4ccccc4)nc3c3c2oc2ccccc23)cc1]

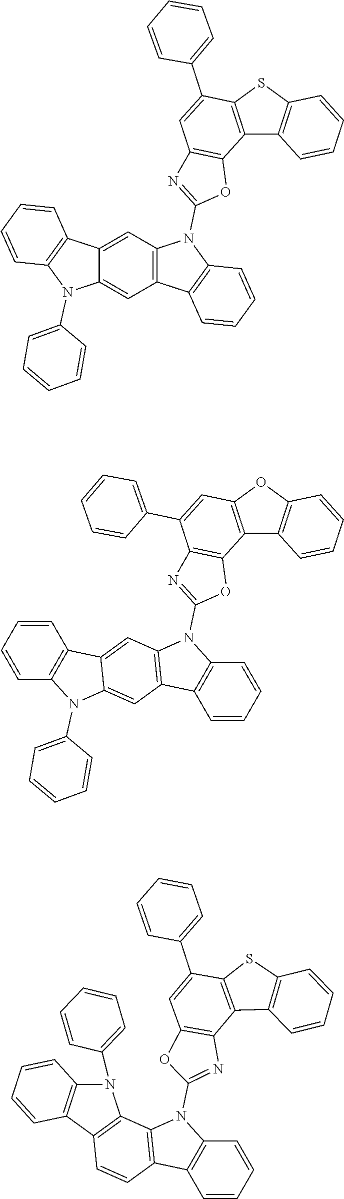 [SMILES: c1ccc(-c2cc3nc(-n4c5ccccc5c5cc6c(cc54)c4ccccc4n6-c4ccccc4)oc3c3c2sc2ccccc23)cc1 ; c1ccc(-c2cc3oc4ccccc4c3c3oc(-n4c5ccccc5c5cc6c(cc54)c4ccccc4n6-c4ccccc4)nc23)cc1 ; c1ccc(-c2cc3oc(-n4c5ccccc5c5ccc6c7ccccc7n(-c7ccccc7)c6c54)nc3c3c2sc2ccccc23)cc1]

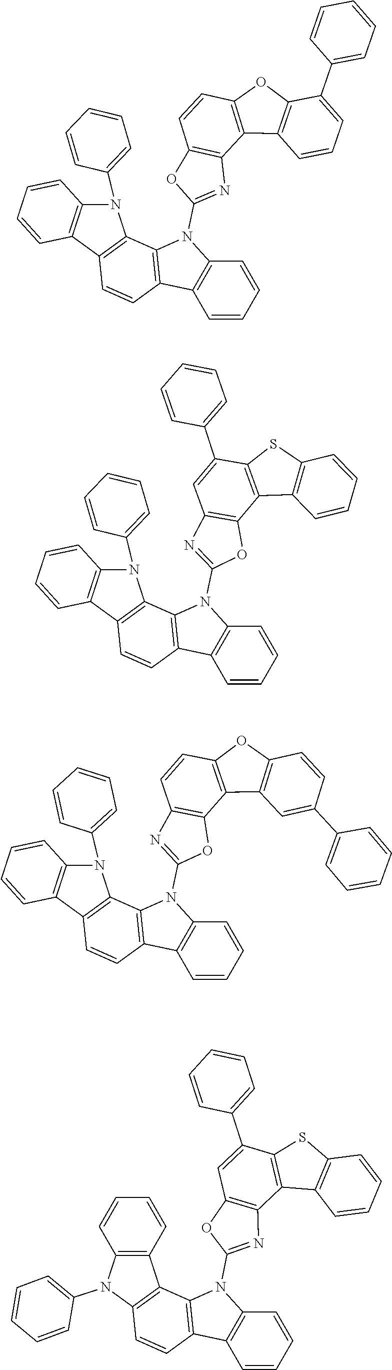 [SMILES: c1ccc(-c2cccc3c2oc2ccc4oc(-n5c6ccccc6c6ccc7c8ccccc8n(-c8ccccc8)c7c65)nc4c23)cc1 ; c1ccc(-c2cc3nc(-n4c5ccccc5c5ccc6c7ccccc7n(-c7ccccc7)c6c54)oc3c3c2sc2ccccc23)cc1 ; c1ccc(-c2ccc3oc4ccc5nc(-n6c7ccccc7c7ccc8c9ccccc9n(-c9ccccc9)c8c76)oc5c4c3c2)cc1 ; c1ccc(-c2cc3oc(-n4c5ccccc5c5ccc6c(c7ccccc7n6-c6ccccc6)c54)nc3c3c2sc2ccccc23)cc1]

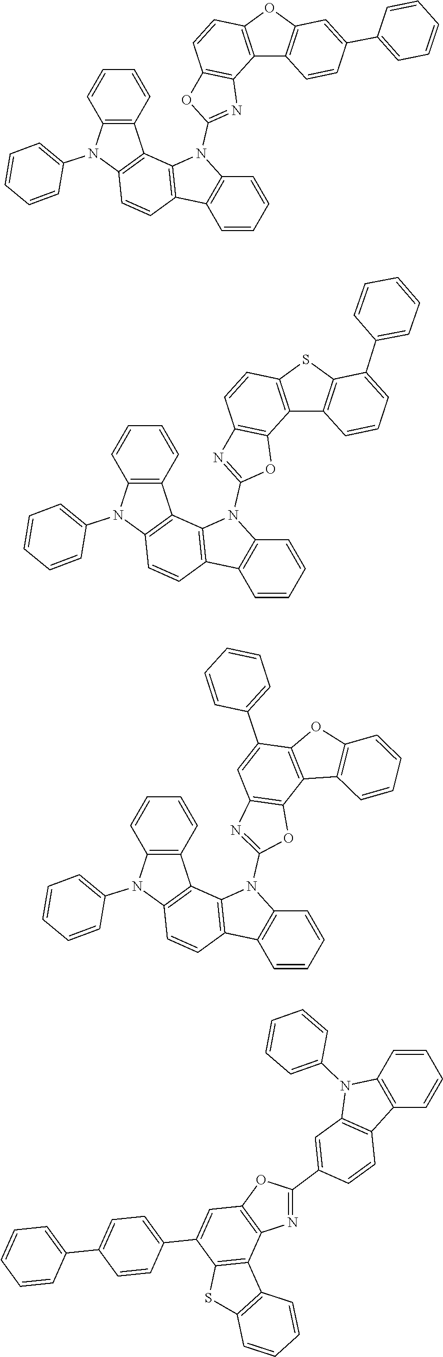

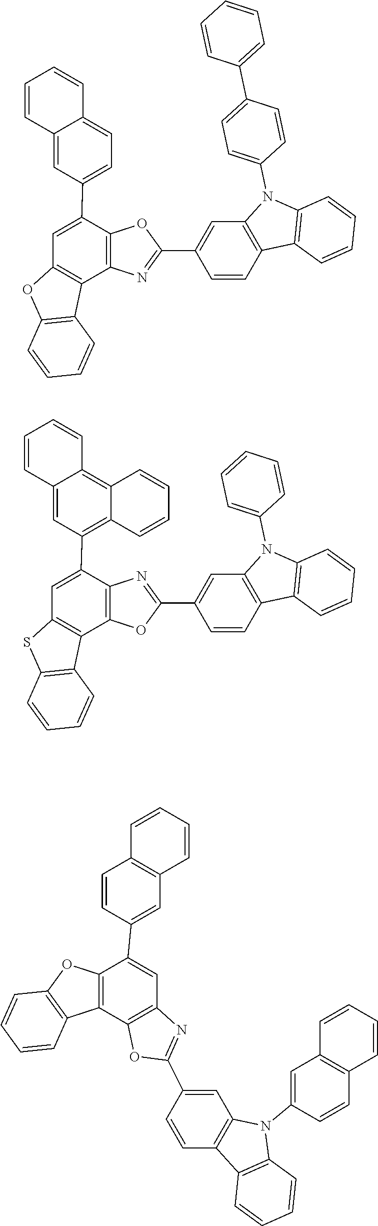

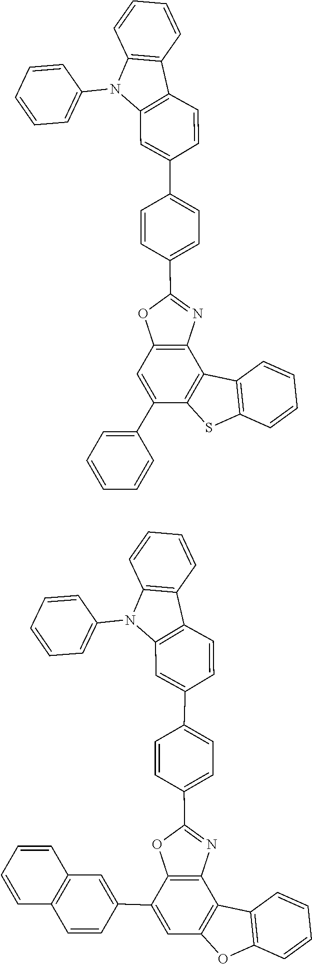

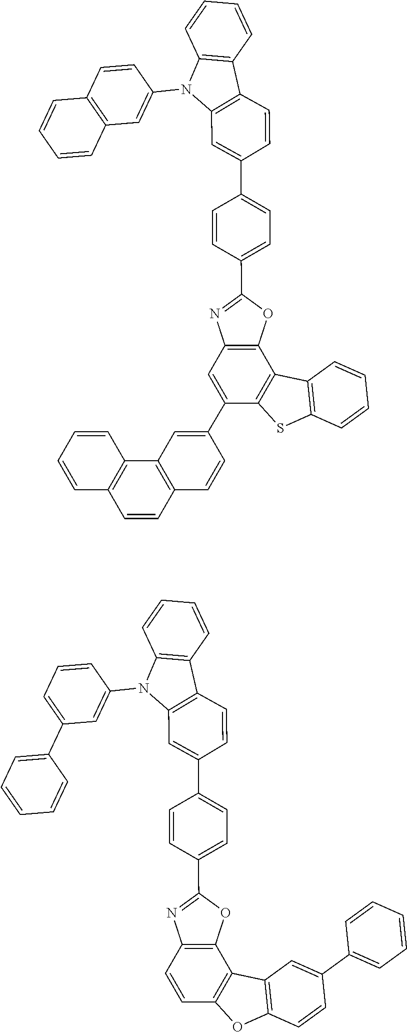 [SMILES: c1ccc2cc(-n3c4ccccc4c4ccc(-c5ccc(-c6nc7cc(-c8ccc9ccc%10ccccc%10c9c8)c8sc9ccccc9c8c7o6)cc5)cc43)ccc2c1 ; c1ccc(-c2cccc(-n3c4ccccc4c4ccc(-c5ccc(-c6nc7ccc8oc9ccc(-c%10ccccc%10)cc9c8c7o6)cc5)cc43)c2)cc1]

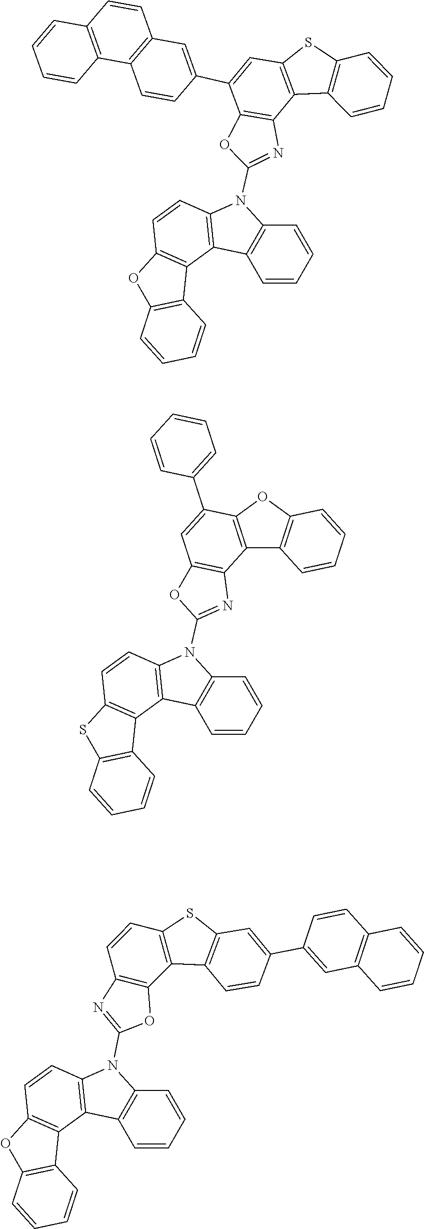 [SMILES: c1ccc2c(c1)ccc1cc(-c3cc4sc5ccccc5c4c4nc(-n5c6ccccc6c6c7c(ccc65)oc5ccccc57)oc34)ccc12 ; c1ccc(-c2cc3oc(-n4c5ccccc5c5c6c(ccc54)sc4ccccc46)nc3c3c2oc2ccccc23)cc1 ; c1ccc2cc(-c3ccc4c(c3)sc3ccc5nc(-n6c7ccccc7c7c8c(ccc76)oc6ccccc68)oc5c34)ccc2c1]

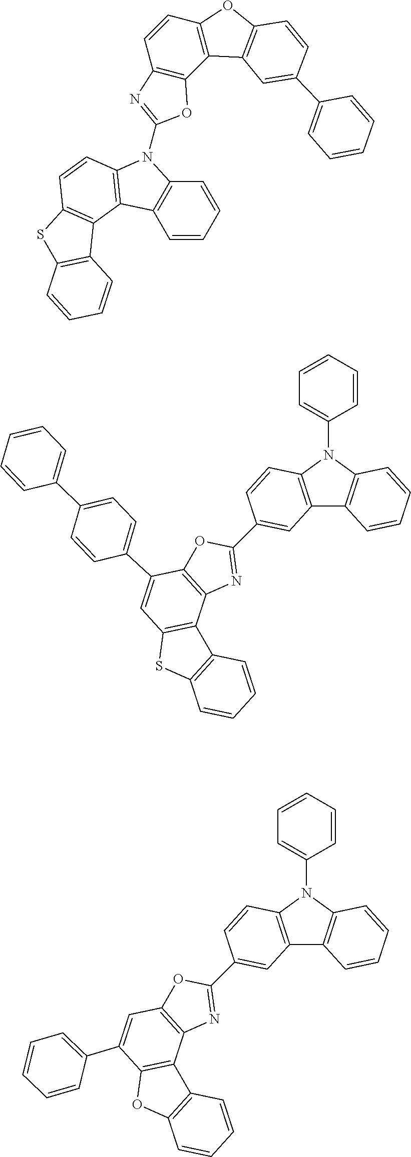 [SMILES: c1ccc(-c2ccc3oc4ccc5nc(-n6c7ccccc7c7c8c(ccc76)sc6ccccc68)oc5c4c3c2)cc1 ; c1ccc(-c2ccc(-c3cc4sc5ccccc5c4c4nc(-c5ccc6c(c5)c5ccccc5n6-c5ccccc5)oc34)cc2)cc1 ; c1ccc(-c2cc3oc(-c4ccc5c(c4)c4ccccc4n5-c4ccccc4)nc3c3c2oc2ccccc23)cc1]

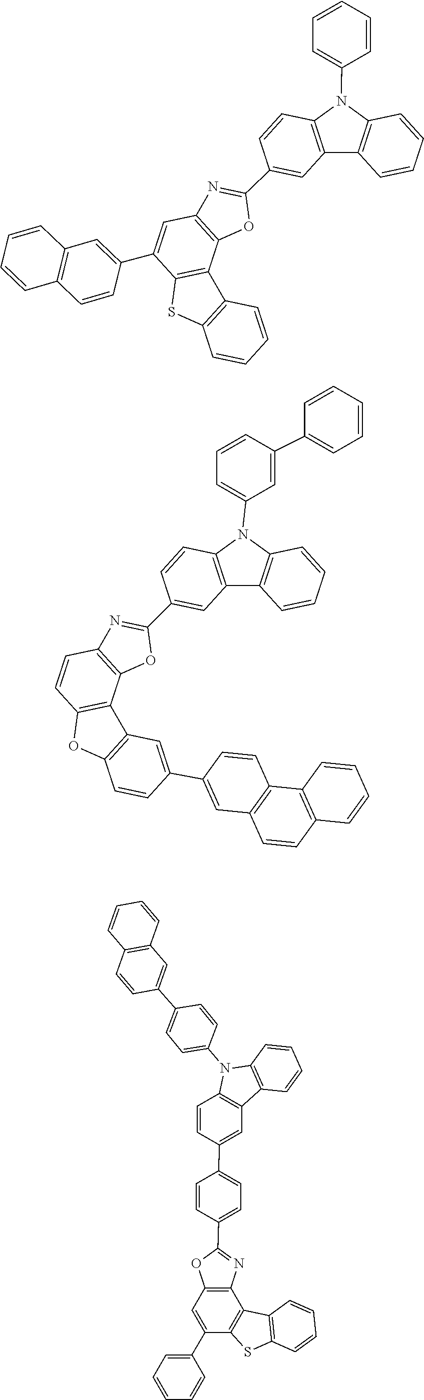 [SMILES: c1ccc(-n2c3ccccc3c3cc(-c4nc5cc(-c6ccc7ccccc7c6)c6sc7ccccc7c6c5o4)ccc32)cc1 ; c1ccc(-c2cccc(-n3c4ccccc4c4cc(-c5nc6ccc7oc8ccc(-c9ccc%10c(ccc%11ccccc%11%10)c9)cc8c7c6o5)ccc43)c2)cc1 ; c1ccc(-c2cc3oc(-c4ccc(-c5ccc6c(c5)c5ccccc5n6-c5ccc(-c6ccc7ccccc7c6)cc5)cc4)nc3c3c2sc2ccccc23)cc1]

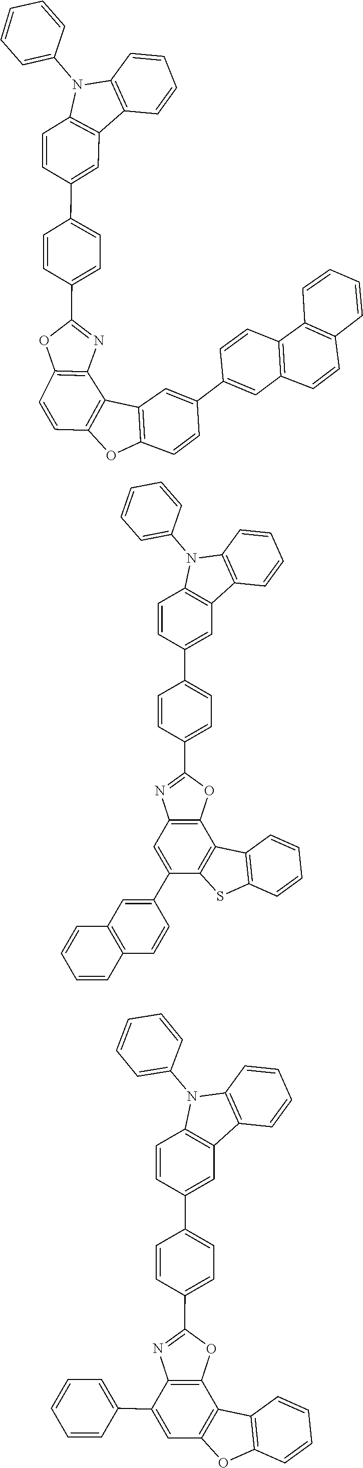

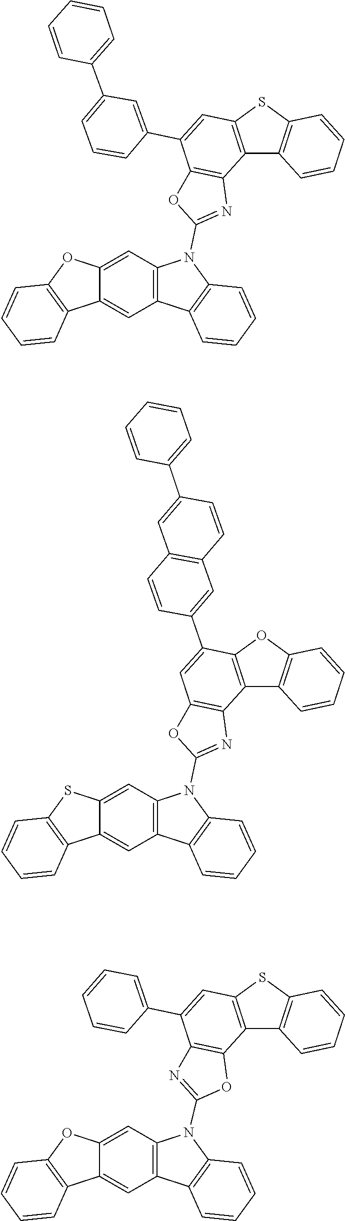 [SMILES: c1ccc(-c2cccc(-c3cc4sc5ccccc5c4c4nc(-n5c6ccccc6c6cc7c(cc65)oc5ccccc57)oc34)c2)cc1 ; c1ccc(-c2ccc3cc(-c4cc5oc(-n6c7ccccc7c7cc8c(cc76)sc6ccccc68)nc5c5c4oc4ccccc45)ccc3c2)cc1 ; c1ccc(-c2cc3sc4ccccc4c3c3oc(-n4c5ccccc5c5cc6c(cc54)oc4ccccc46)nc23)cc1]

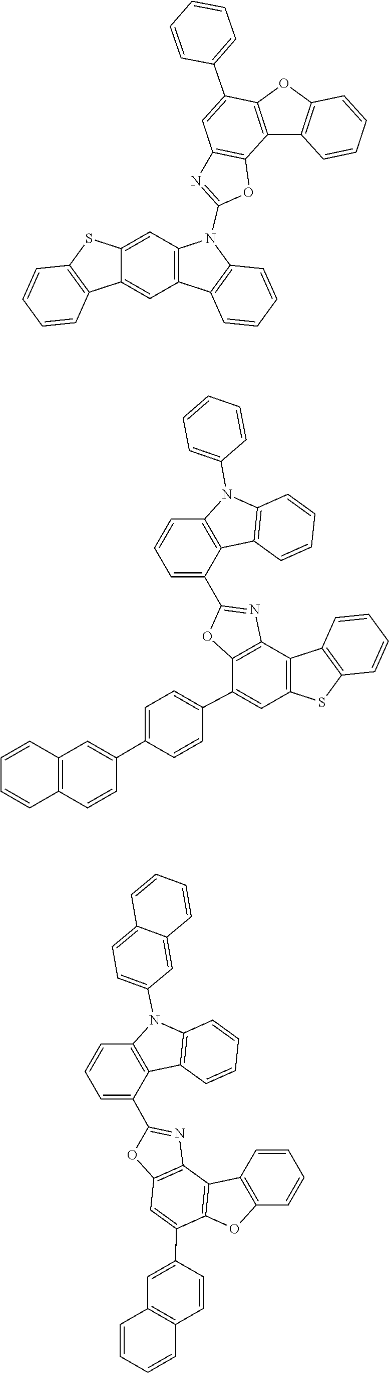 [SMILES: c1ccc(-c2cc3nc(-n4c5ccccc5c5cc6c(cc54)sc4ccccc46)oc3c3c2oc2ccccc23)cc1 ; c1ccc(-n2c3ccccc3c3c(-c4nc5c(o4)c(-c4ccc(-c6ccc7ccccc7c6)cc4)cc4sc6ccccc6c45)cccc32)cc1 ; c1ccc2cc(-c3cc4oc(-c5cccc6c5c5ccccc5n6-c5ccc6ccccc6c5)nc4c4c3oc3ccccc34)ccc2c1]

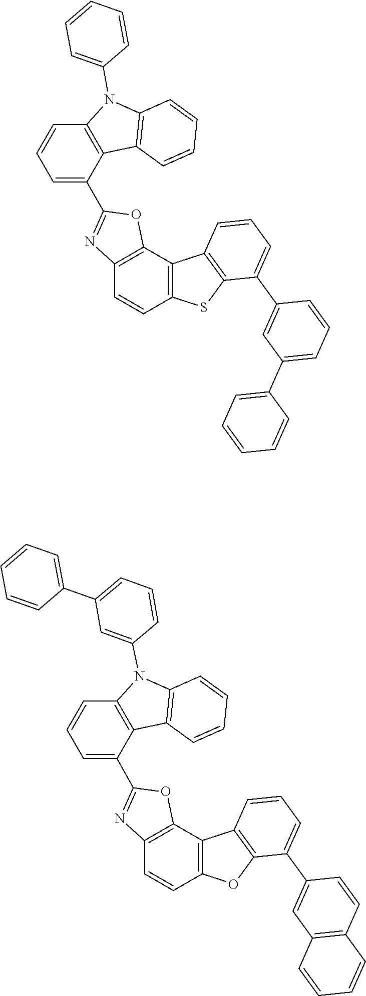 [SMILES: c1ccc(-c2cccc(-c3cccc4c3sc3ccc5nc(-c6cccc7c6c6ccccc6n7-c6ccccc6)oc5c34)c2)cc1 ; c1ccc(-c2cccc(-n3c4ccccc4c4c(-c5nc6ccc7oc8c(-c9ccc%10ccccc%10c9)cccc8c7c6o5)cccc43)c2)cc1]

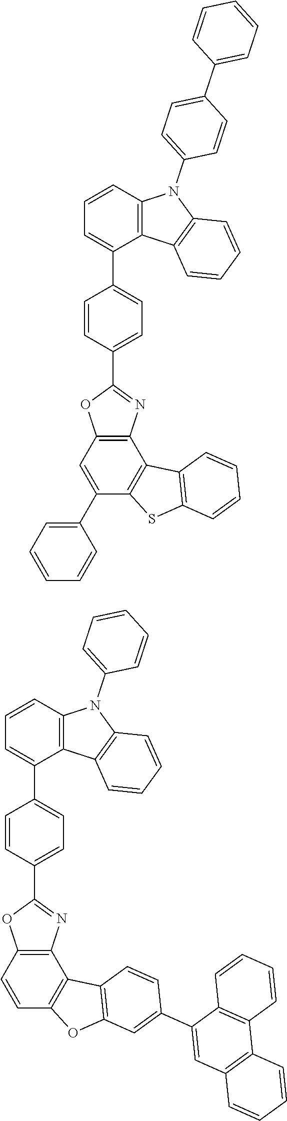

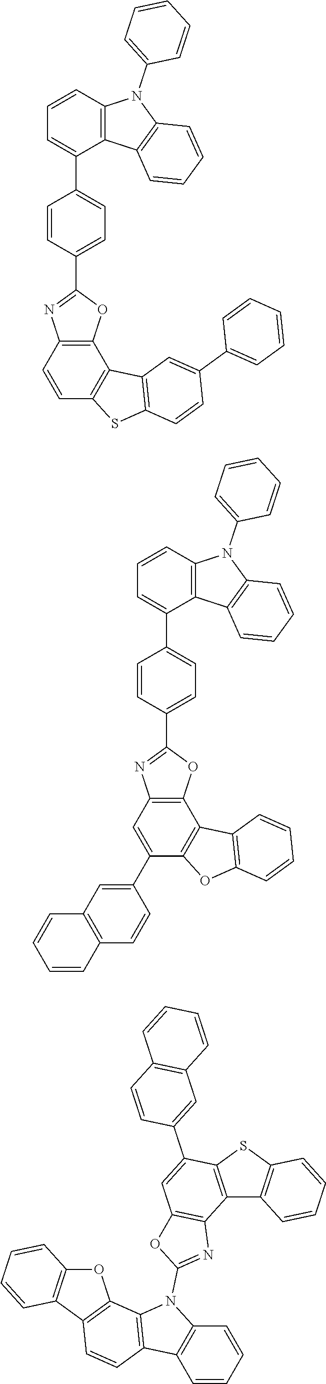 [SMILES: c1ccc(-c2ccc3sc4ccc5nc(-c6ccc(-c7cccc8c7c7ccccc7n8-c7ccccc7)cc6)oc5c4c3c2)cc1 ; c1ccc(-n2c3ccccc3c3c(-c4ccc(-c5nc6cc(-c7ccc8ccccc8c7)c7oc8ccccc8c7c6o5)cc4)cccc32)cc1 ; c1ccc2cc(-c3cc4oc(-n5c6ccccc6c6ccc7c8ccccc8oc7c65)nc4c4c3sc3ccccc34)ccc2c1]

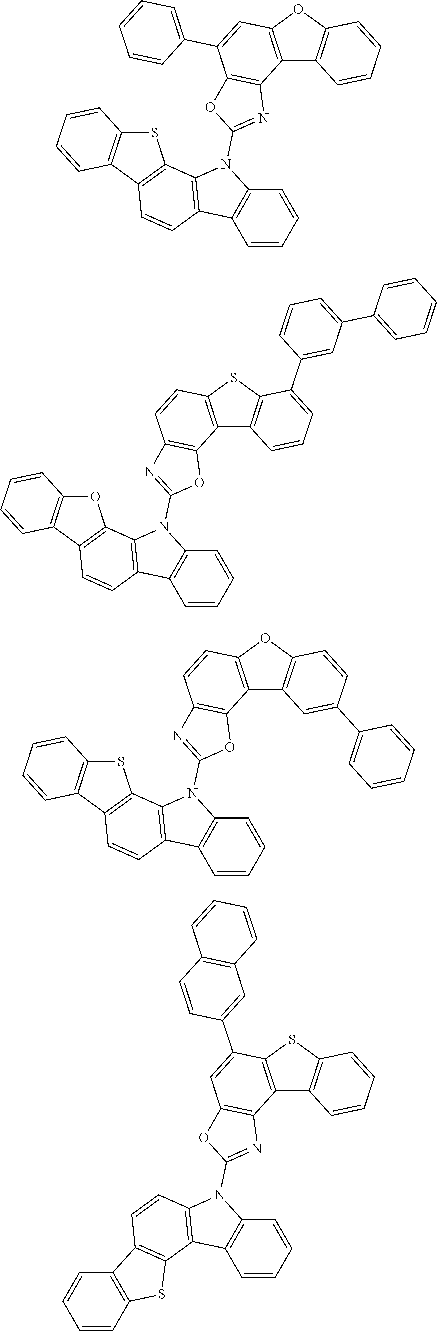

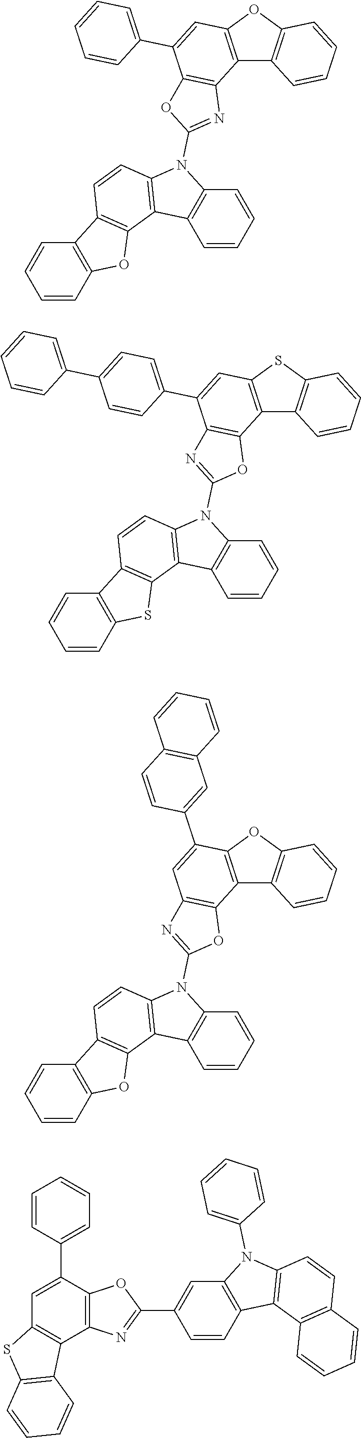

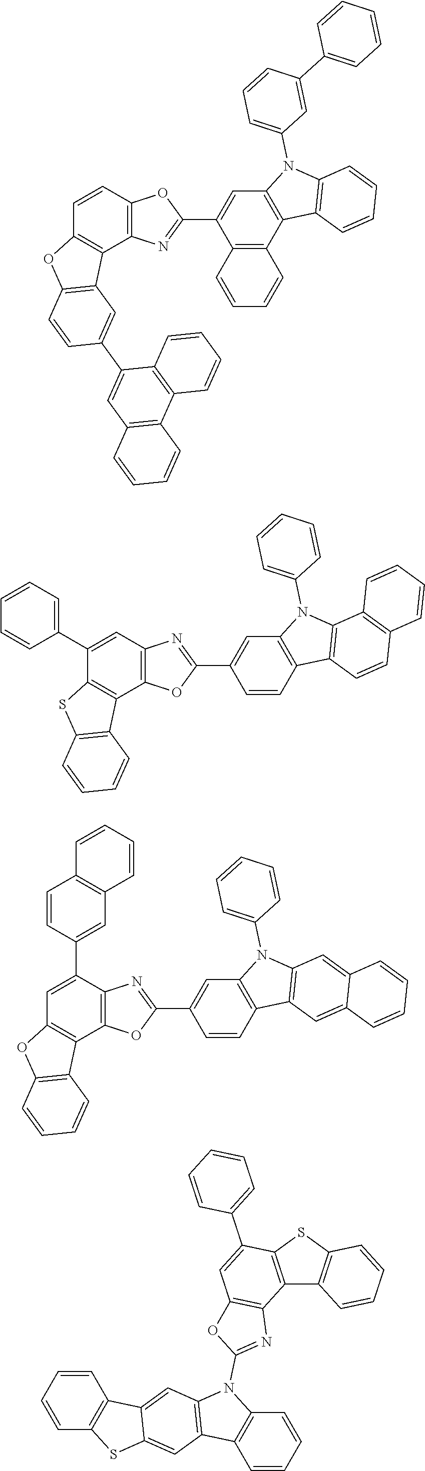

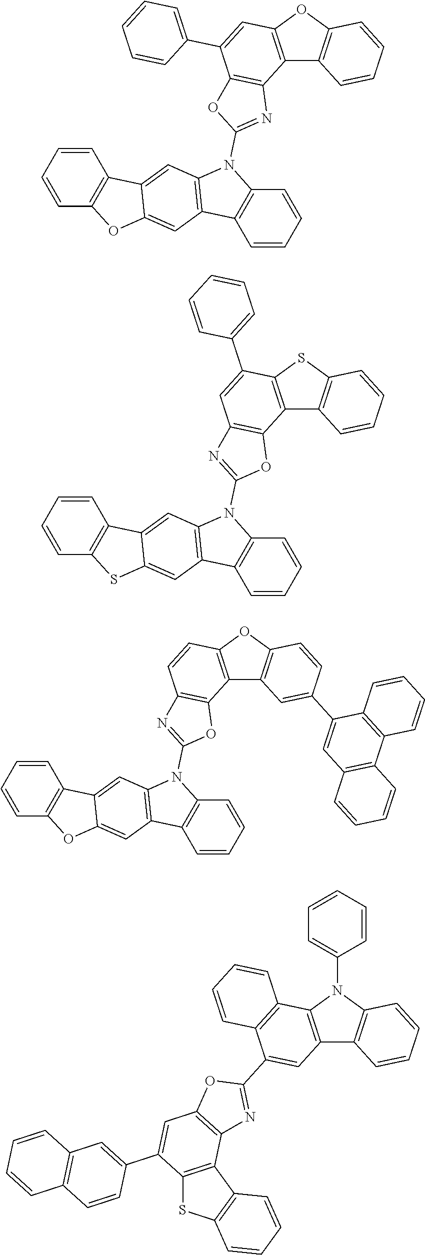

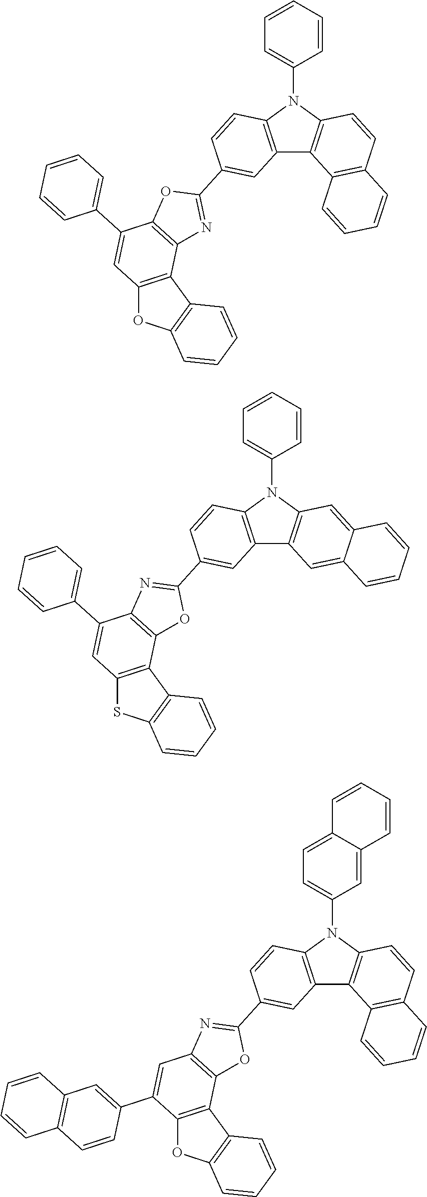 [SMILES: c1ccc(-c2cc3oc4ccccc4c3c3nc(-c4ccc5c(c4)c4c6ccccc6ccc4n5-c4ccccc4)oc23)cc1 ; c1ccc(-c2cc3sc4ccccc4c3c3oc(-c4ccc5c(c4)c4cc6ccccc6cc4n5-c4ccccc4)nc23)cc1 ; c1ccc2cc(-c3cc4nc(-c5ccc6c(c5)c5c7ccccc7ccc5n6-c5ccc6ccccc6c5)oc4c4c3oc3ccccc34)ccc2c1]

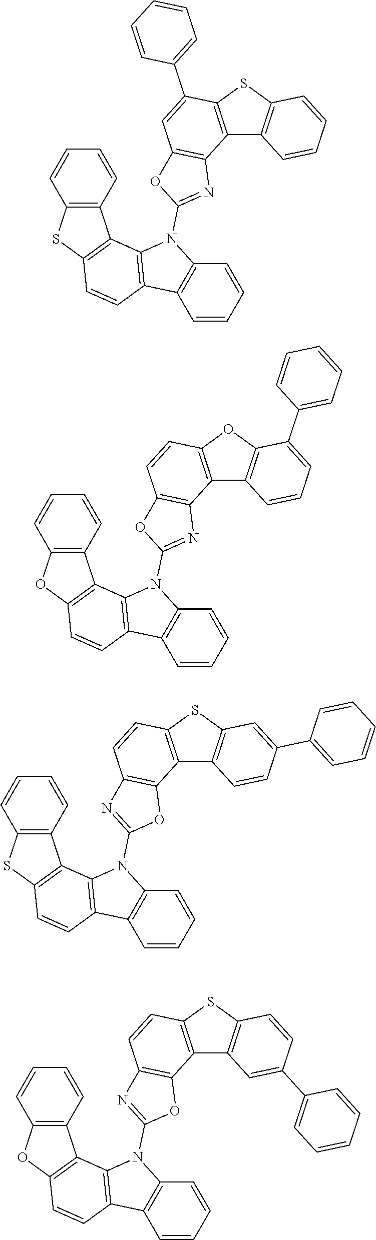

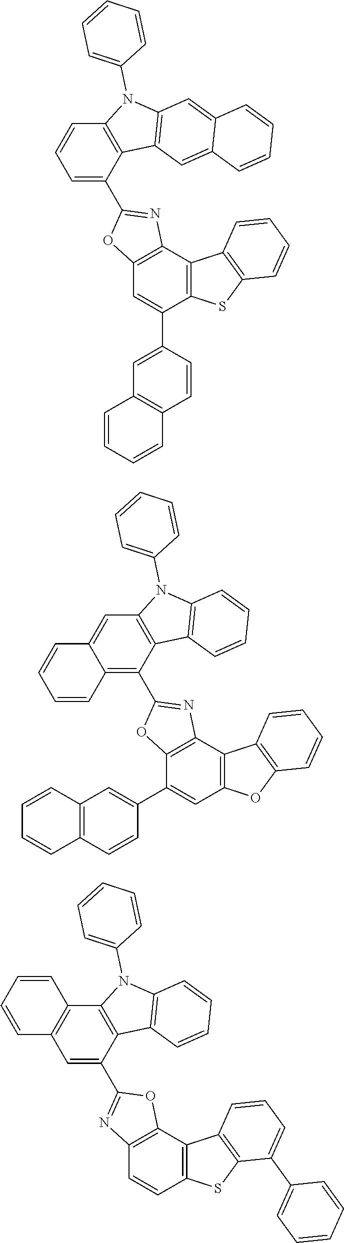

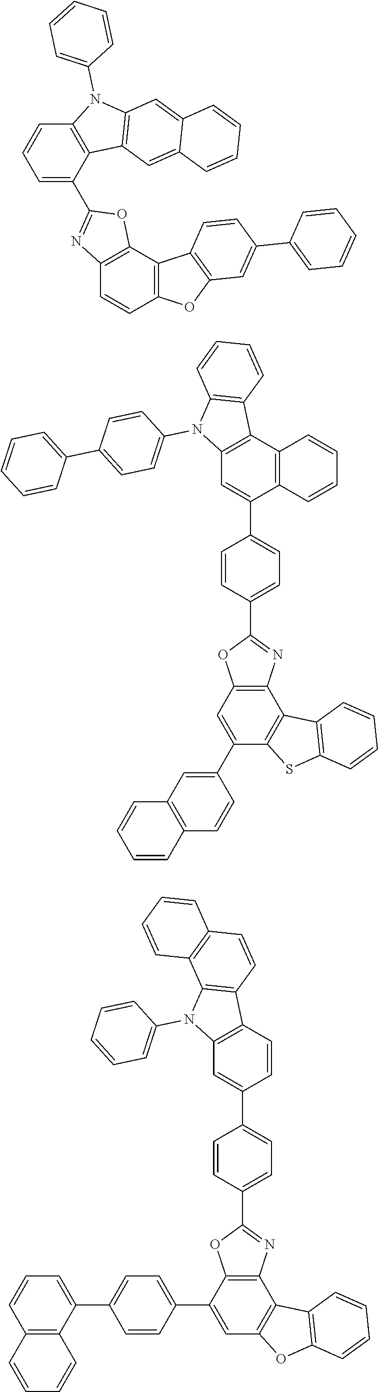

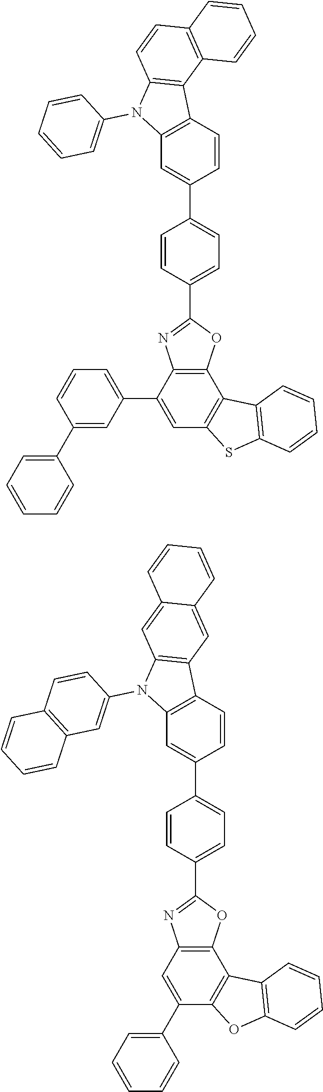 [SMILES: c1ccc(-c2cccc(-c3cc4sc5ccccc5c4c4oc(-c5ccc(-c6ccc7c8c9ccccc9ccc8n(-c8ccccc8)c7c6)cc5)nc34)c2)cc1 ; c1ccc(-c2cc3nc(-c4ccc(-c5ccc6c7cc8ccccc8cc7n(-c7ccc8ccccc8c7)c6c5)cc4)oc3c3c2oc2ccccc23)cc1]

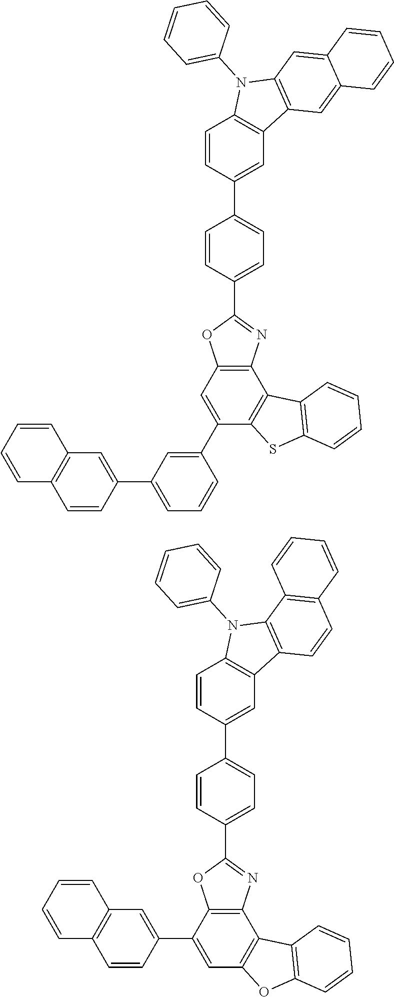

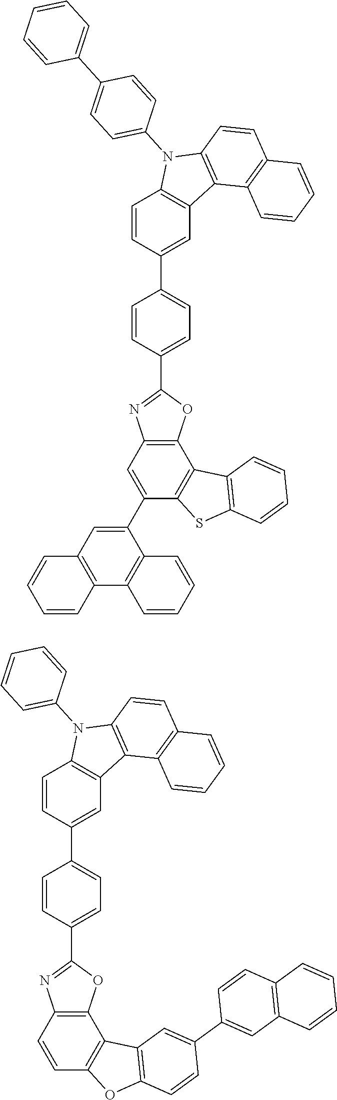

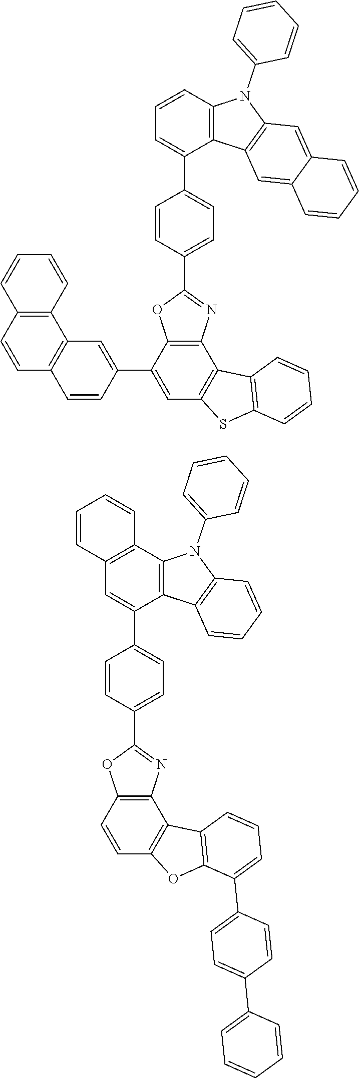

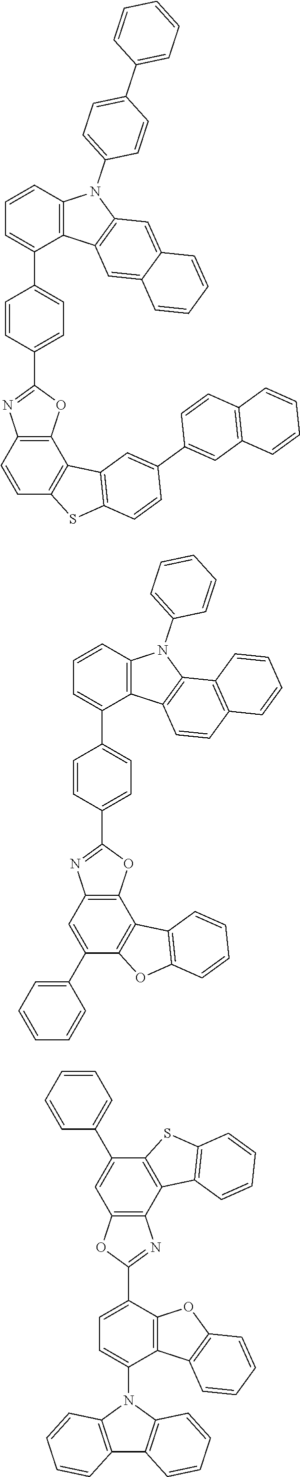

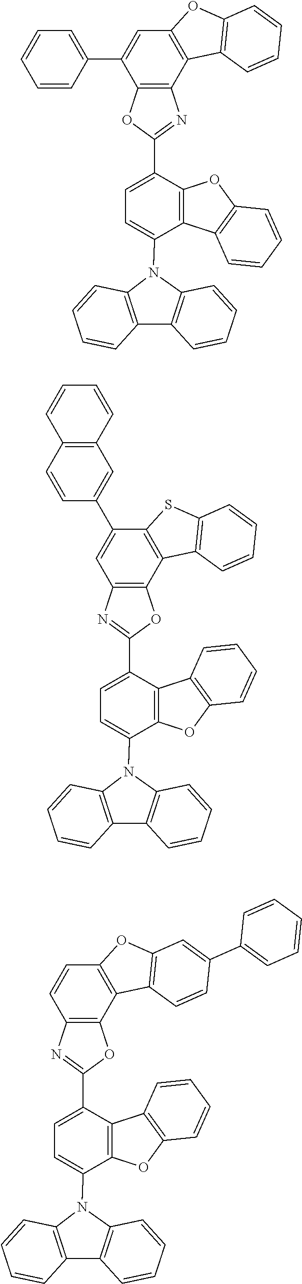

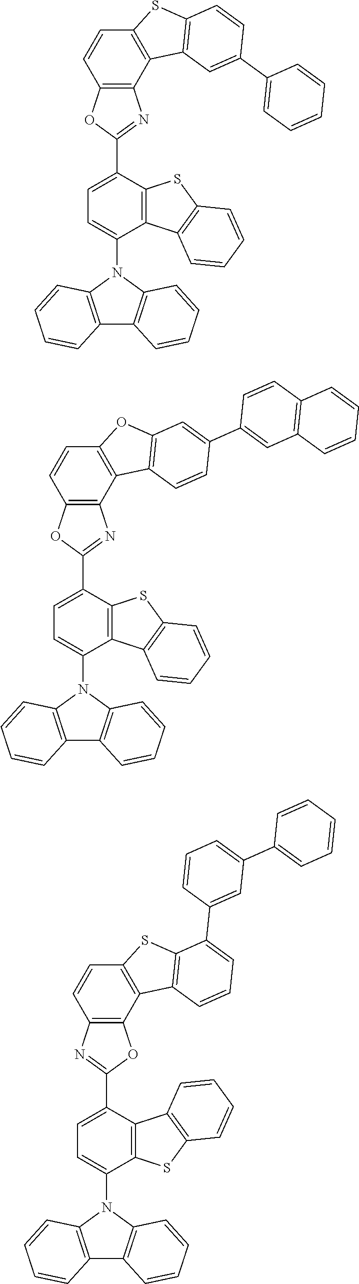 [SMILES: c1ccc(-c2ccc3sc4ccc5oc(-c6ccc(-n7c8ccccc8c8ccccc87)c7c6sc6ccccc67)nc5c4c3c2)cc1 ; c1ccc2cc(-c3ccc4c(c3)oc3ccc5oc(-c6ccc(-n7c8ccccc8c8ccccc87)c7c6sc6ccccc67)nc5c34)ccc2c1 ; c1ccc(-c2cccc(-c3cccc4c3sc3ccc5nc(-c6ccc(-n7c8ccccc8c8ccccc87)c7sc8ccccc8c67)oc5c34)c2)cc1]

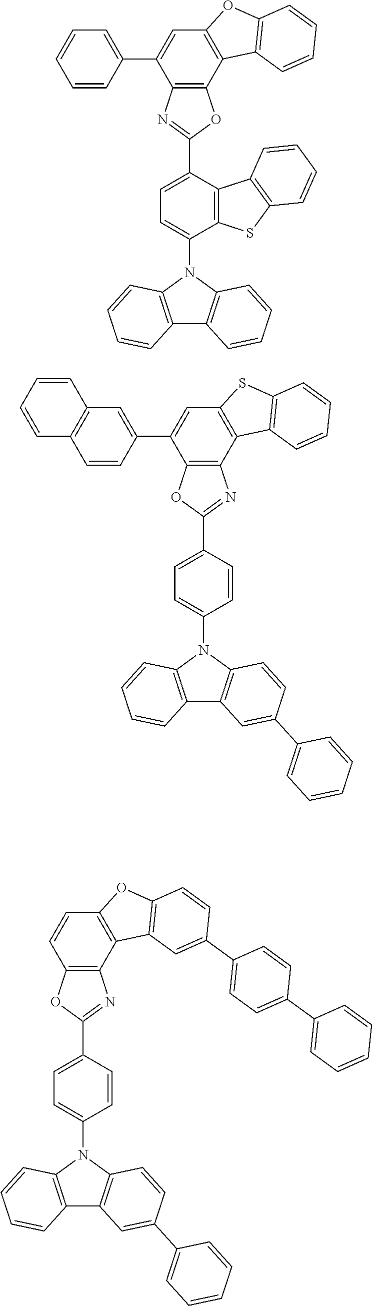

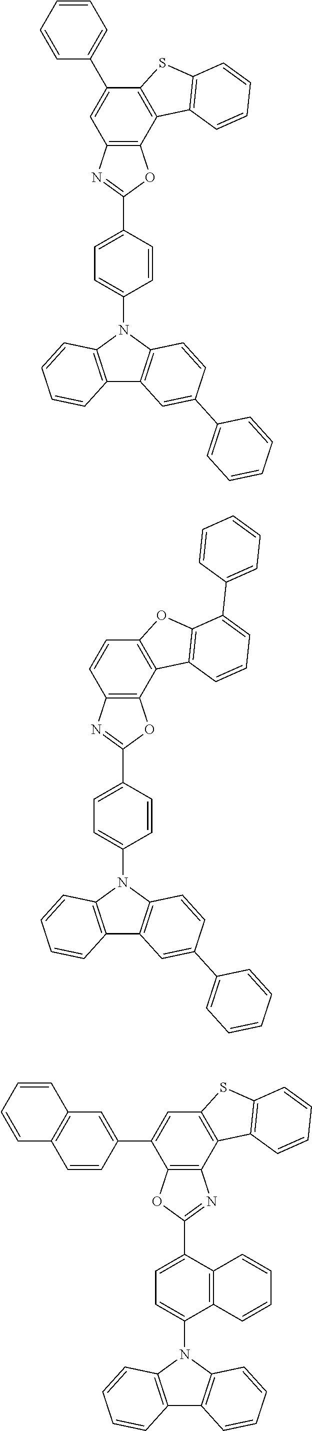

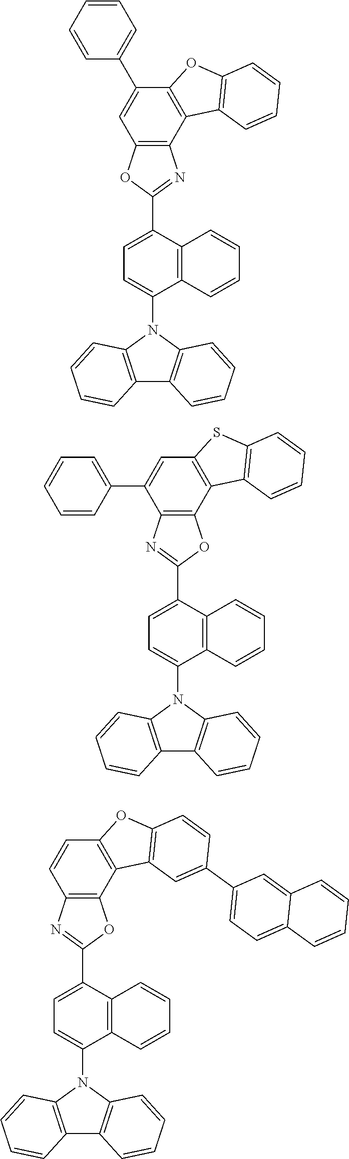

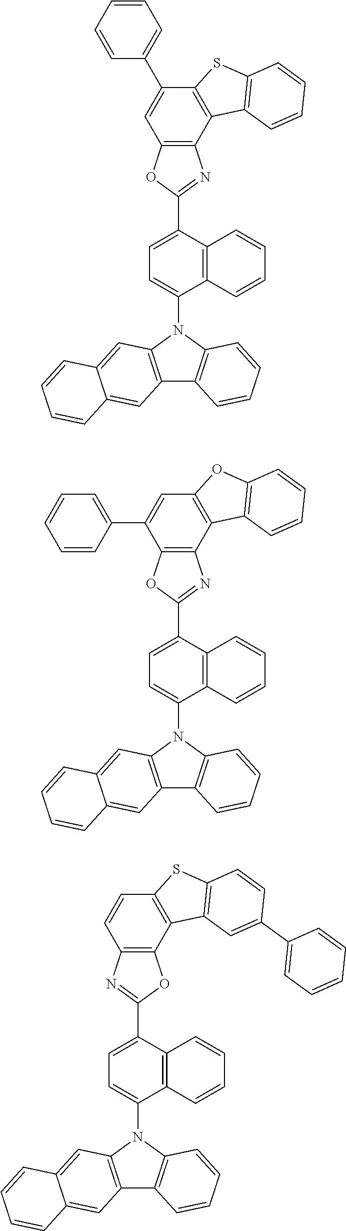 [SMILES: c1ccc(-c2cc3oc(-c4ccc(-n5c6ccccc6c6cc7ccccc7cc65)c5ccccc45)nc3c3c2sc2ccccc23)cc1 ; c1ccc(-c2cc3oc4ccccc4c3c3nc(-c4ccc(-n5c6ccccc6c6cc7ccccc7cc65)c5ccccc45)oc23)cc1 ; c1ccc(-c2ccc3sc4ccc5nc(-c6ccc(-n7c8ccccc8c8cc9ccccc9cc87)c7ccccc67)oc5c4c3c2)cc1]

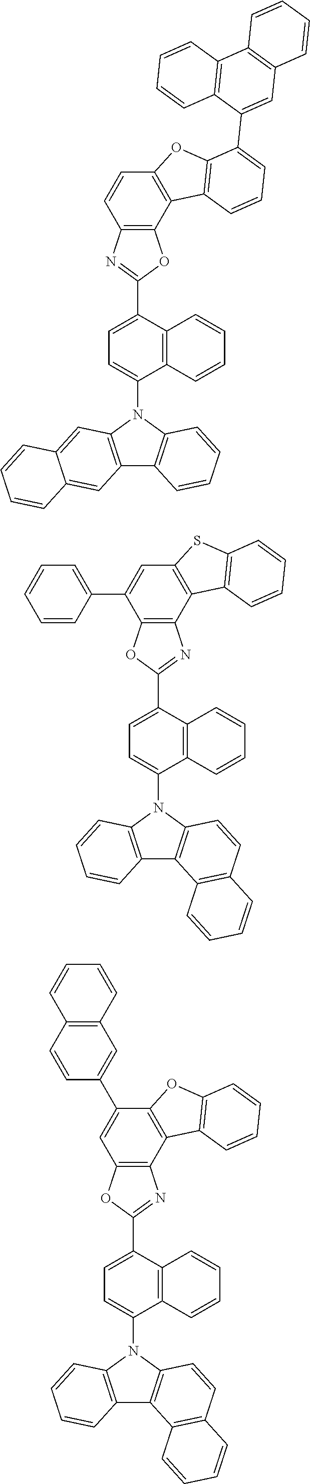 [SMILES: c1ccc2cc3c(cc2c1)c1ccccc1n3-c1ccc(-c2nc3ccc4oc5c(-c6cc7ccccc7c7ccccc67)cccc5c4c3o2)c2ccccc12 ; c1ccc(-c2cc3sc4ccccc4c3c3nc(-c4ccc(-n5c6ccccc6c6c7ccccc7ccc65)c5ccccc45)oc23)cc1 ; c1ccc2cc(-c3cc4oc(-c5ccc(-n6c7ccccc7c7c8ccccc8ccc76)c6ccccc56)nc4c4c3oc3ccccc34)ccc2c1]

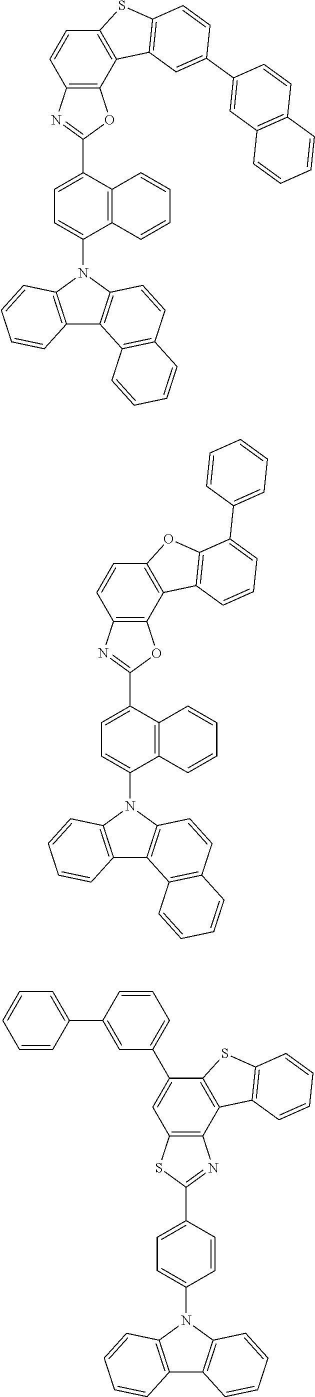 [SMILES: c1ccc2cc(-c3ccc4sc5ccc6nc(-c7ccc(-n8c9ccccc9c9c%10ccccc%10ccc98)c8ccccc78)oc6c5c4c3)ccc2c1 ; c1ccc(-c2cccc3c2oc2ccc4nc(-c5ccc(-n6c7ccccc7c7c8ccccc8ccc76)c6ccccc56)oc4c23)cc1 ; c1ccc(-c2cccc(-c3cc4sc(-c5ccc(-n6c7ccccc7c7ccccc76)cc5)nc4c4c3sc3ccccc34)c2)cc1]

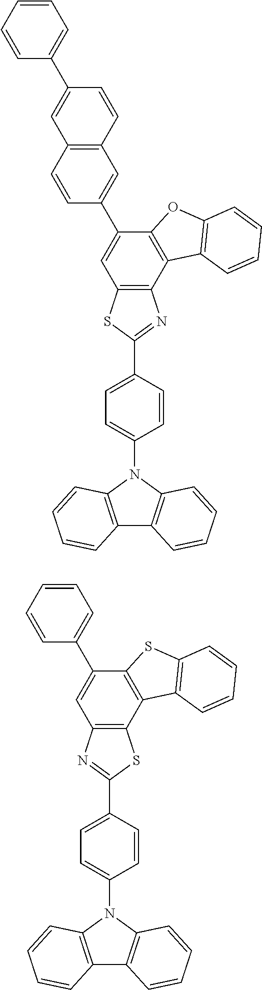

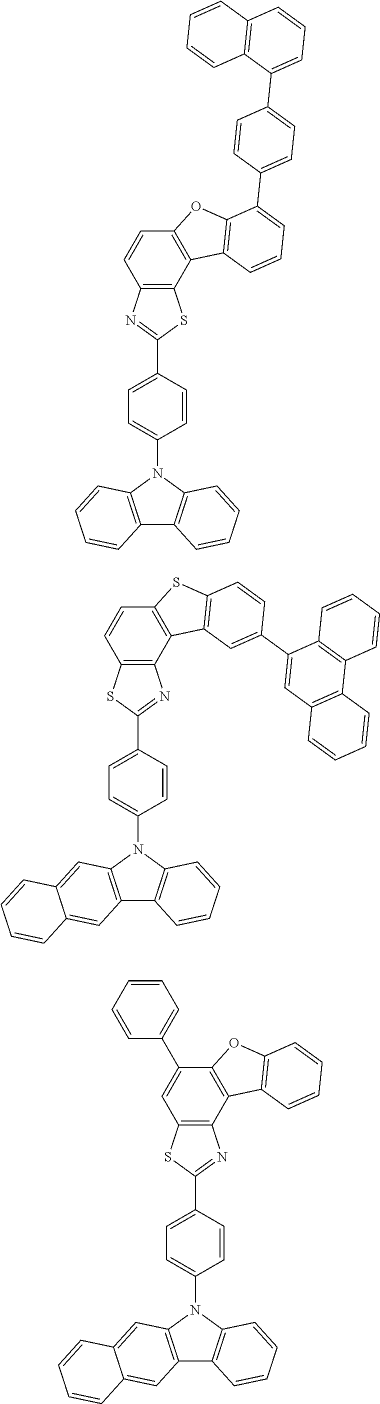

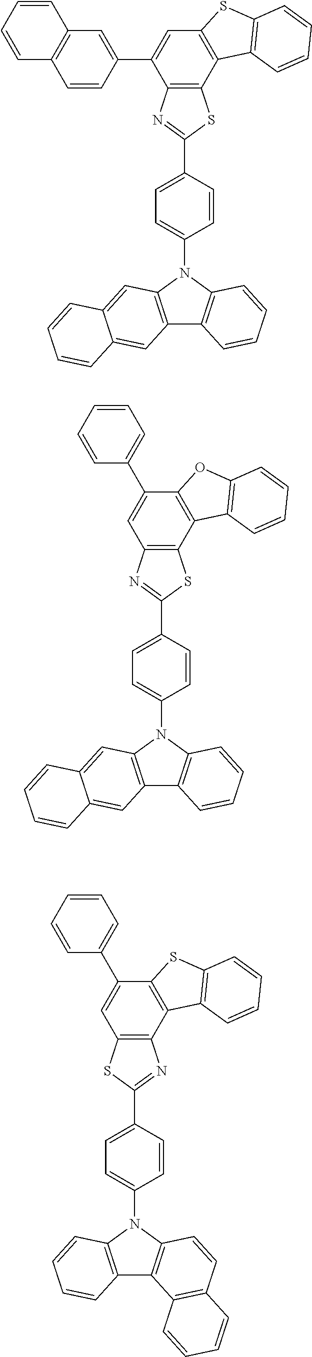

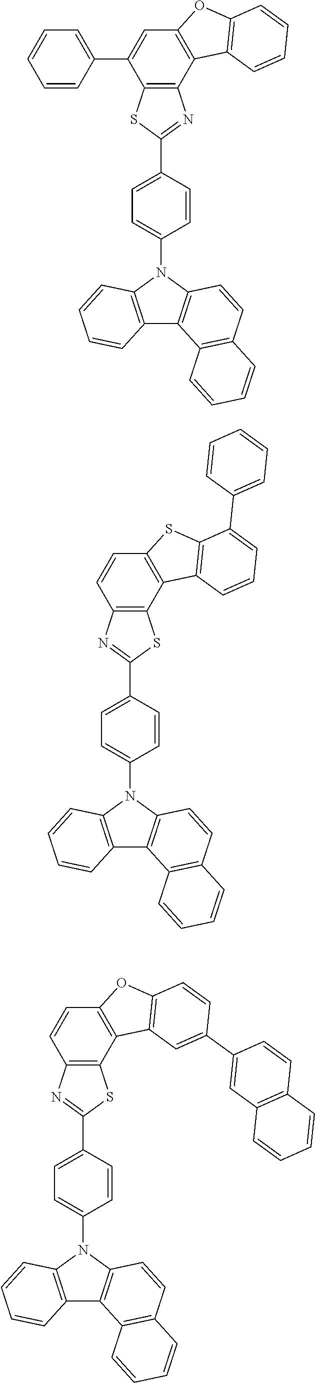

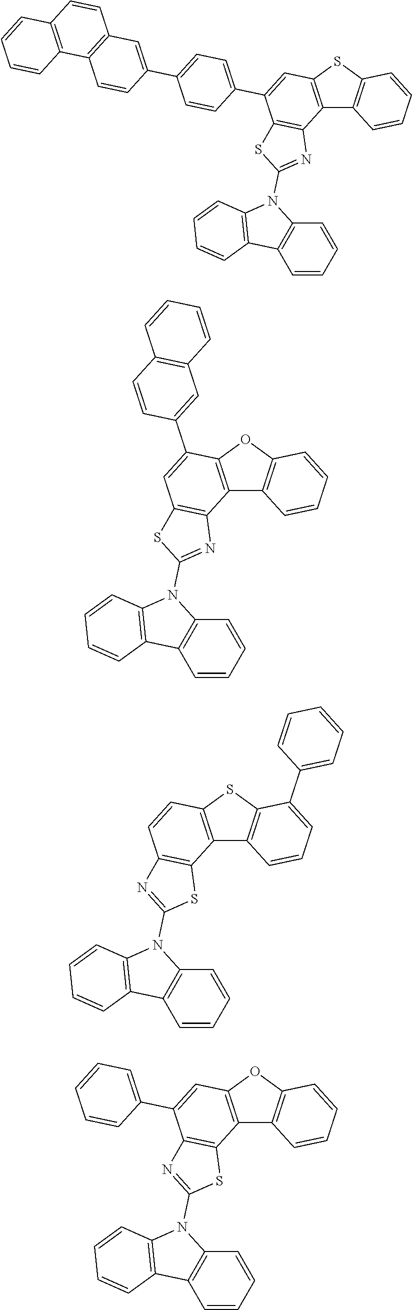 [SMILES: c1ccc2c(c1)ccc1cc(-c3ccc(-c4cc5sc6ccccc6c5c5nc(-n6c7ccccc7c7ccccc76)sc45)cc3)ccc12 ; c1ccc2cc(-c3cc4sc(-n5c6ccccc6c6ccccc65)nc4c4c3oc3ccccc34)ccc2c1 ; c1ccc(-c2cccc3c2sc2ccc4nc(-n5c6ccccc6c6ccccc65)sc4c23)cc1 ; c1ccc(-c2cc3oc4ccccc4c3c3sc(-n4c5ccccc5c5ccccc54)nc23)cc1]

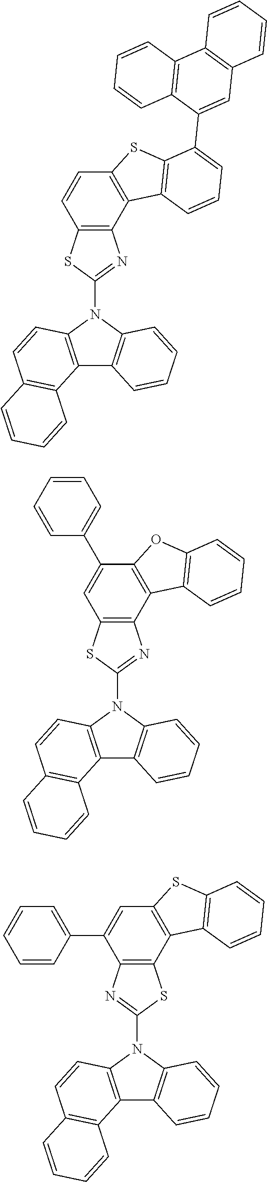

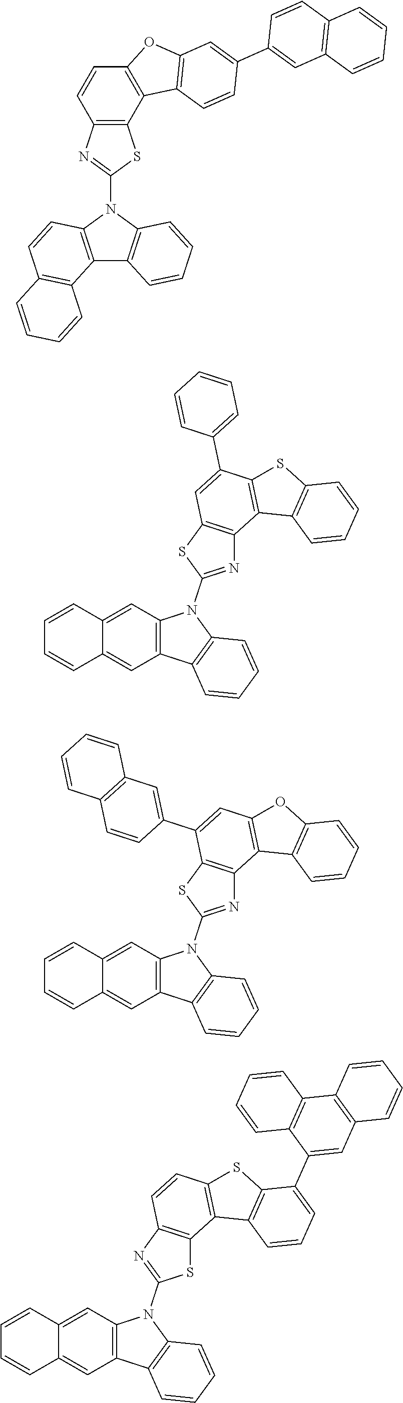

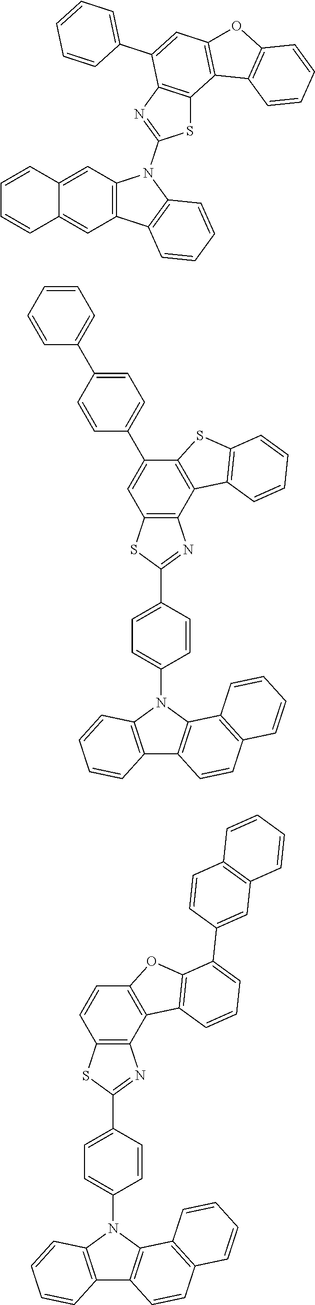 [SMILES: c1ccc(-c2cc3oc4ccccc4c3c3sc(-n4c5ccccc5c5cc6ccccc6cc54)nc23)cc1 ; c1ccc(-c2ccc(-c3cc4sc(-c5ccc(-n6c7ccccc7c7ccc8ccccc8c76)cc5)nc4c4c3sc3ccccc34)cc2)cc1 ; c1ccc2cc(-c3cccc4c3oc3ccc5sc(-c6ccc(-n7c8ccccc8c8ccc9ccccc9c87)cc6)nc5c34)ccc2c1]

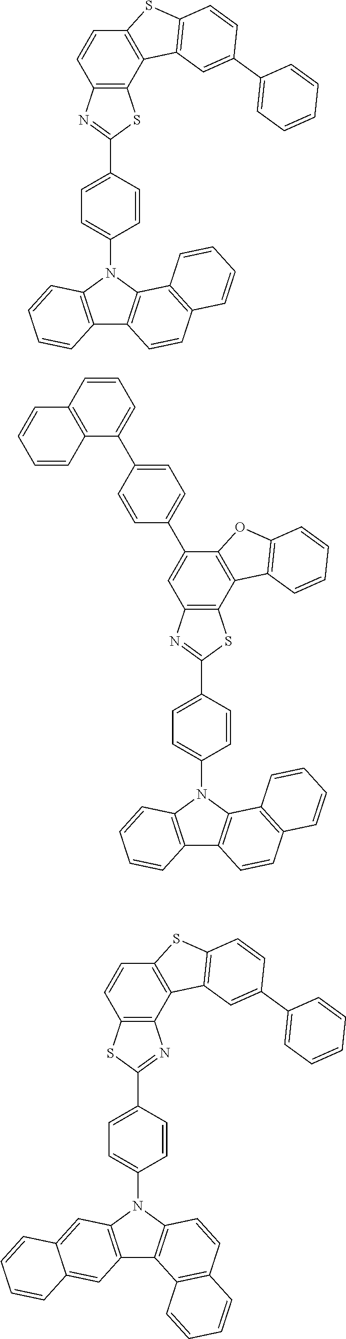

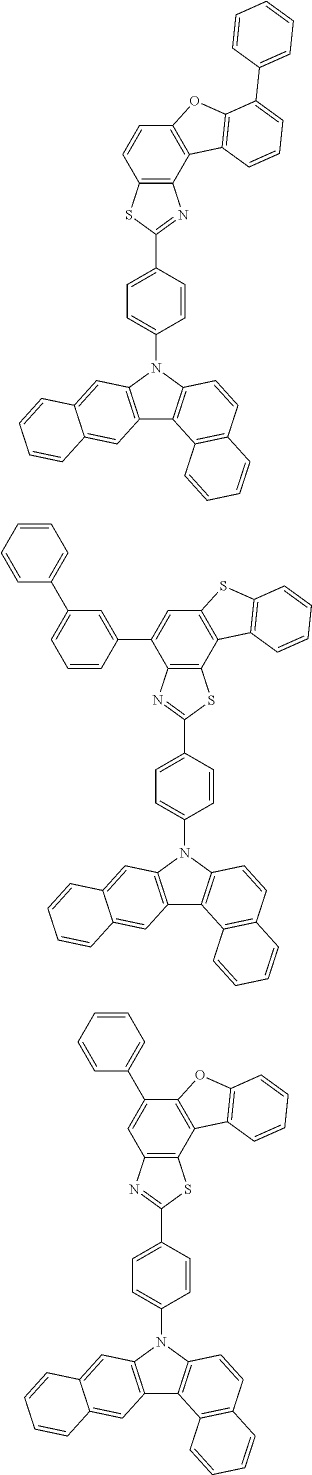 [SMILES: c1ccc(-c2cccc3c2oc2ccc4sc(-c5ccc(-n6c7cc8ccccc8cc7c7c8ccccc8ccc76)cc5)nc4c23)cc1 ; c1ccc(-c2cccc(-c3cc4sc5ccccc5c4c4sc(-c5ccc(-n6c7cc8ccccc8cc7c7c8ccccc8ccc76)cc5)nc34)c2)cc1 ; c1ccc(-c2cc3nc(-c4ccc(-n5c6cc7ccccc7cc6c6c7ccccc7ccc65)cc4)sc3c3c2oc2ccccc23)cc1]

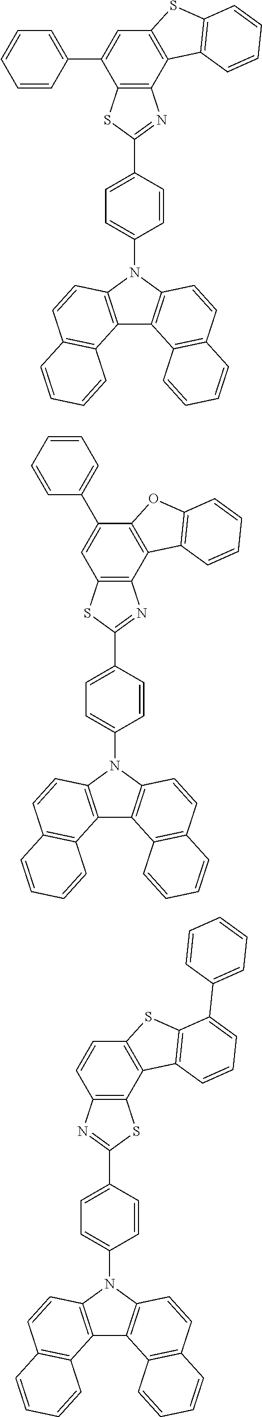

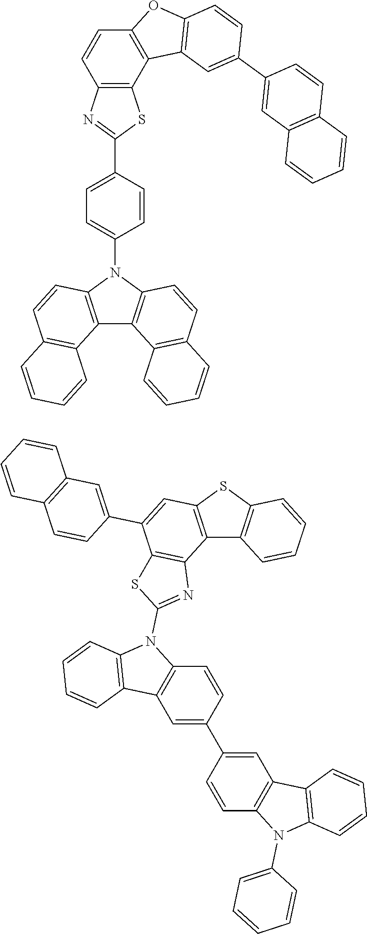

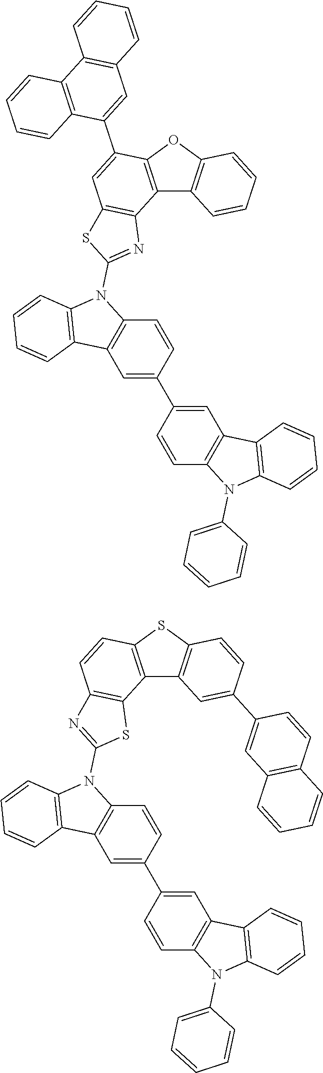 [SMILES: c1ccc(-n2c3ccccc3c3cc(-c4ccc5c(c4)c4ccccc4n5-c4nc5c(cc(-c6cc7ccccc7c7ccccc67)c6oc7ccccc7c65)s4)ccc32)cc1 ; c1ccc(-n2c3ccccc3c3cc(-c4ccc5c(c4)c4ccccc4n5-c4nc5ccc6sc7ccc(-c8ccc9ccccc9c8)cc7c6c5s4)ccc32)cc1]

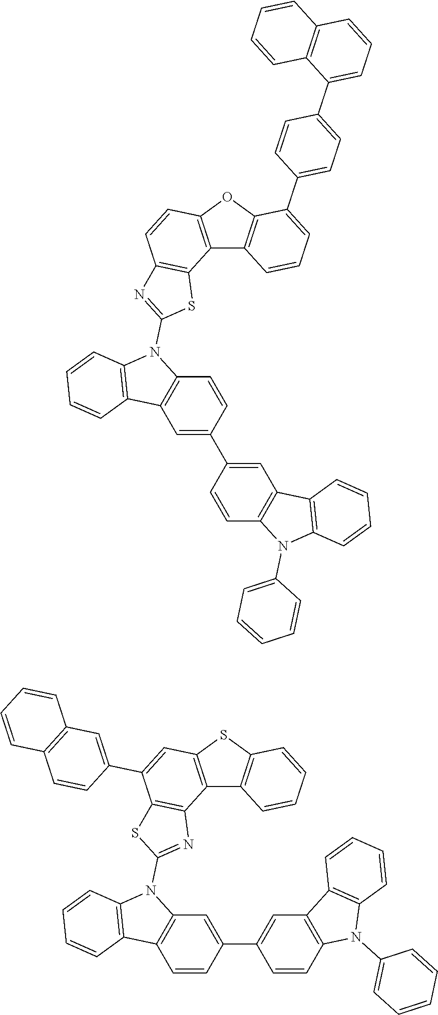 [SMILES: c1ccc(-n2c3ccccc3c3cc(-c4ccc5c(c4)c4ccccc4n5-c4nc5ccc6oc7c(-c8ccc(-c9cccc%10ccccc9%10)cc8)cccc7c6c5s4)ccc32)cc1 ; c1ccc(-n2c3ccccc3c3cc(-c4ccc5c6ccccc6n(-c6nc7c(s6)c(-c6ccc8ccccc8c6)cc6sc8ccccc8c67)c5c4)ccc32)cc1]

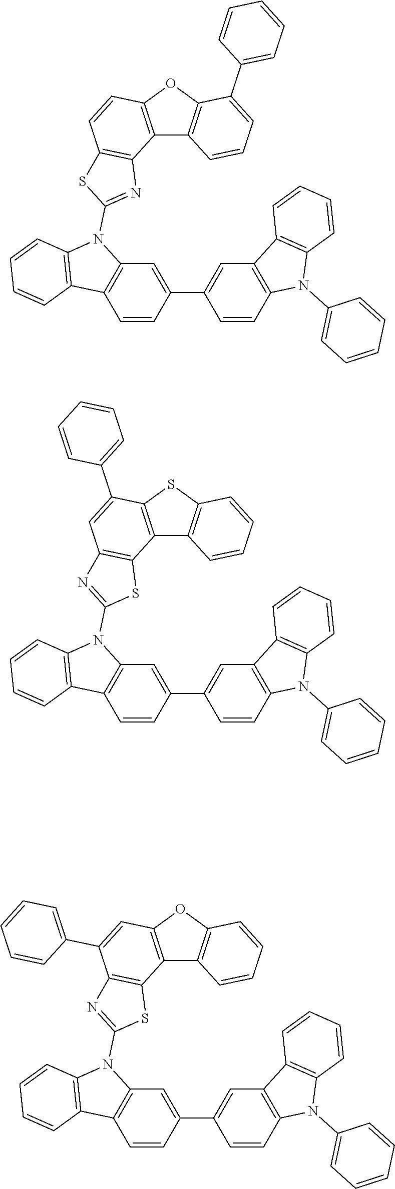 [SMILES: c1ccc(-c2cccc3c2oc2ccc4sc(-n5c6ccccc6c6ccc(-c7ccc8c(c7)c7ccccc7n8-c7ccccc7)cc65)nc4c23)cc1 ; c1ccc(-c2cc3nc(-n4c5ccccc5c5ccc(-c6ccc7c(c6)c6ccccc6n7-c6ccccc6)cc54)sc3c3c2sc2ccccc23)cc1 ; c1ccc(-c2cc3oc4ccccc4c3c3sc(-n4c5ccccc5c5ccc(-c6ccc7c(c6)c6ccccc6n7-c6ccccc6)cc54)nc23)cc1]

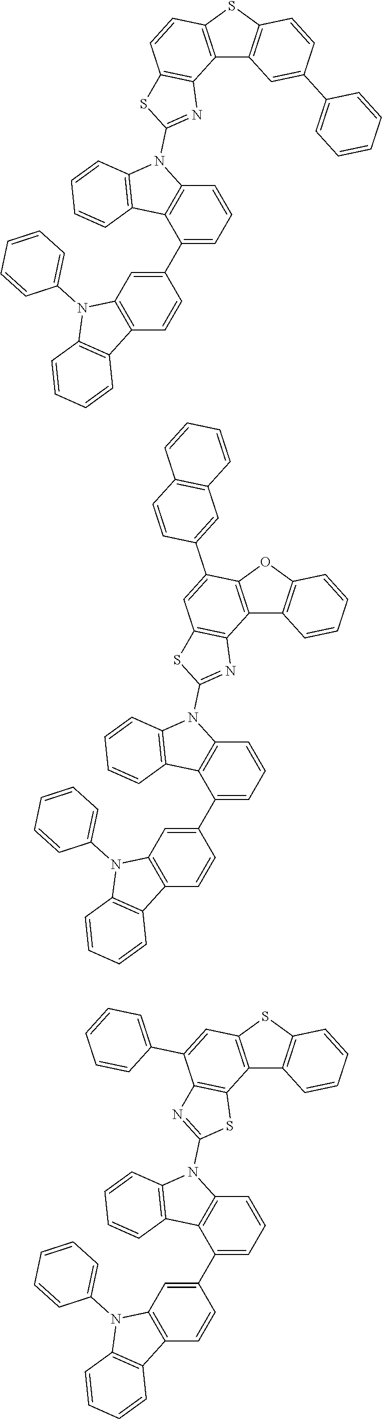

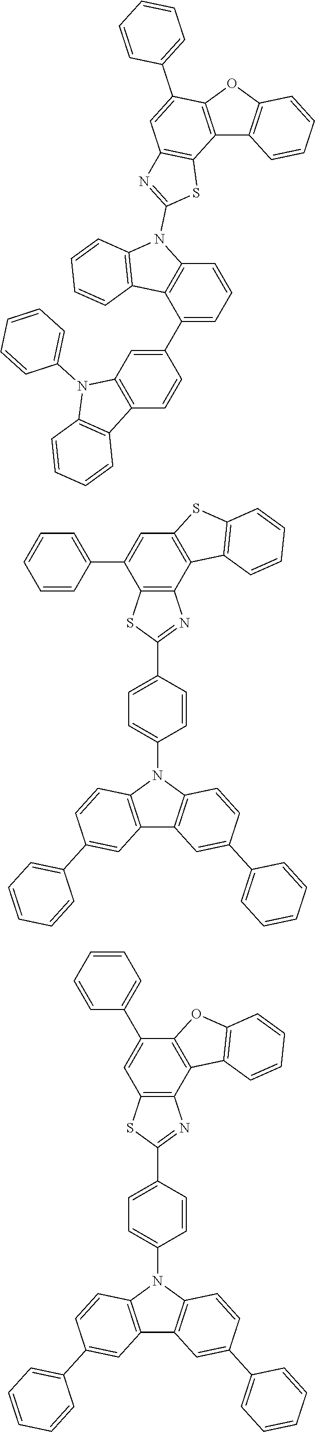 [SMILES: c1ccc(-c2cc3nc(-n4c5ccccc5c5c(-c6ccc7c8ccccc8n(-c8ccccc8)c7c6)cccc54)sc3c3c2oc2ccccc23)cc1 ; c1ccc(-c2ccc3c(c2)c2cc(-c4ccccc4)ccc2n3-c2ccc(-c3nc4c(s3)c(-c3ccccc3)cc3sc5ccccc5c34)cc2)cc1 ; c1ccc(-c2ccc3c(c2)c2cc(-c4ccccc4)ccc2n3-c2ccc(-c3nc4c(cc(-c5ccccc5)c5oc6ccccc6c54)s3)cc2)cc1]

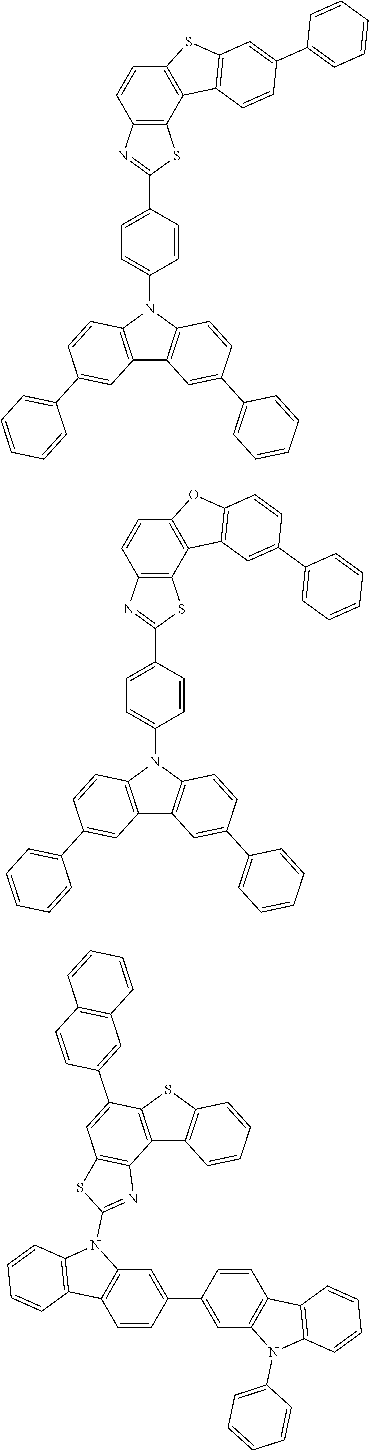

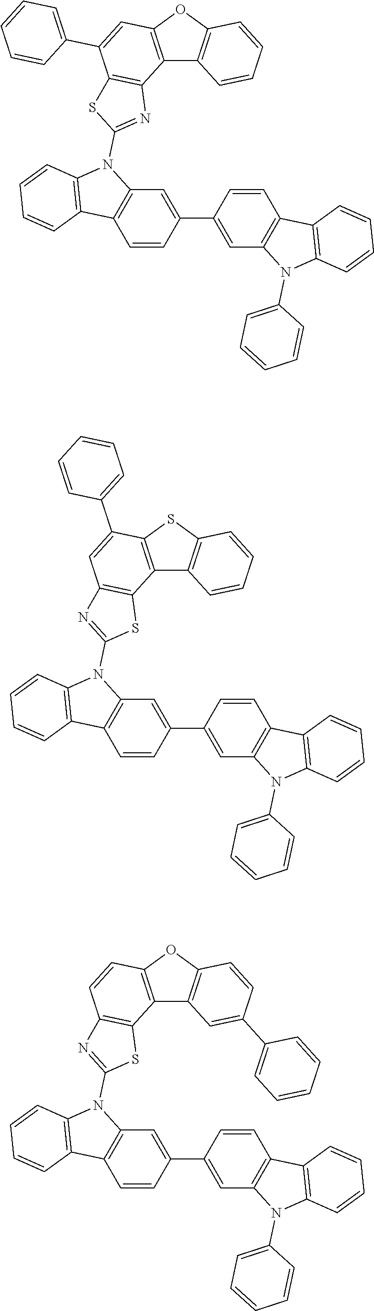 [SMILES: c1ccc(-c2cc3oc4ccccc4c3c3nc(-n4c5ccccc5c5ccc(-c6ccc7c8ccccc8n(-c8ccccc8)c7c6)cc54)sc23)cc1 ; c1ccc(-c2cc3nc(-n4c5ccccc5c5ccc(-c6ccc7c8ccccc8n(-c8ccccc8)c7c6)cc54)sc3c3c2sc2ccccc23)cc1 ; c1ccc(-c2ccc3oc4ccc5nc(-n6c7ccccc7c7ccc(-c8ccc9c%10ccccc%10n(-c%10ccccc%10)c9c8)cc76)sc5c4c3c2)cc1]

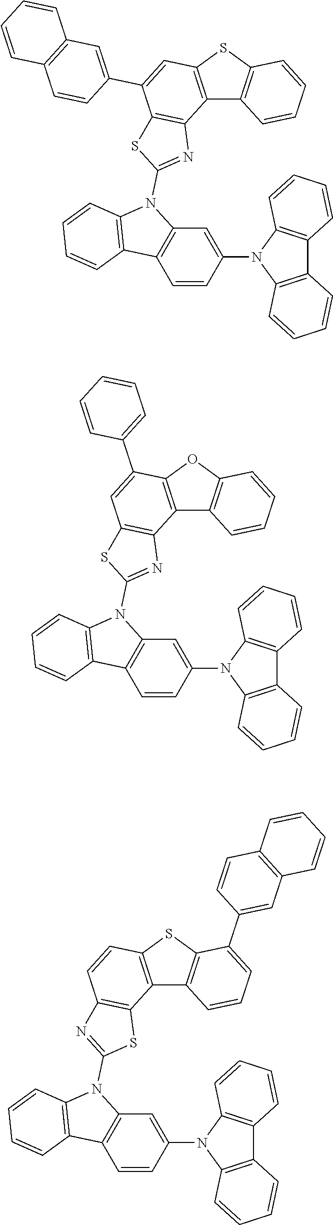 [SMILES: c1ccc2cc(-c3cc4sc5ccccc5c4c4nc(-n5c6ccccc6c6ccc(-n7c8ccccc8c8ccccc87)cc65)sc34)ccc2c1 ; c1ccc(-c2cc3sc(-n4c5ccccc5c5ccc(-n6c7ccccc7c7ccccc76)cc54)nc3c3c2oc2ccccc23)cc1 ; c1ccc2cc(-c3cccc4c3sc3ccc5nc(-n6c7ccccc7c7ccc(-n8c9ccccc9c9ccccc98)cc76)sc5c34)ccc2c1]

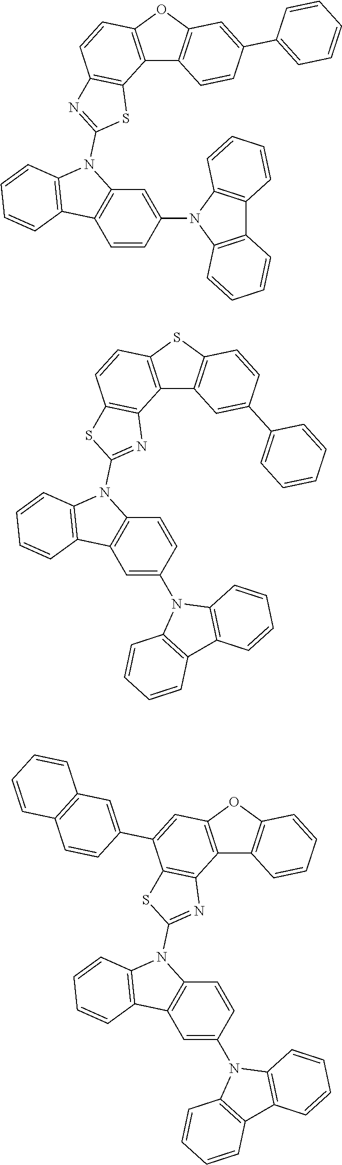 [SMILES: c1ccc(-c2ccc3c(c2)oc2ccc4nc(-n5c6ccccc6c6ccc(-n7c8ccccc8c8ccccc87)cc65)sc4c23)cc1 ; c1ccc(-c2ccc3sc4ccc5sc(-n6c7ccccc7c7cc(-n8c9ccccc9c9ccccc98)ccc76)nc5c4c3c2)cc1 ; c1ccc2cc(-c3cc4oc5ccccc5c4c4nc(-n5c6ccccc6c6cc(-n7c8ccccc8c8ccccc87)ccc65)sc34)ccc2c1]

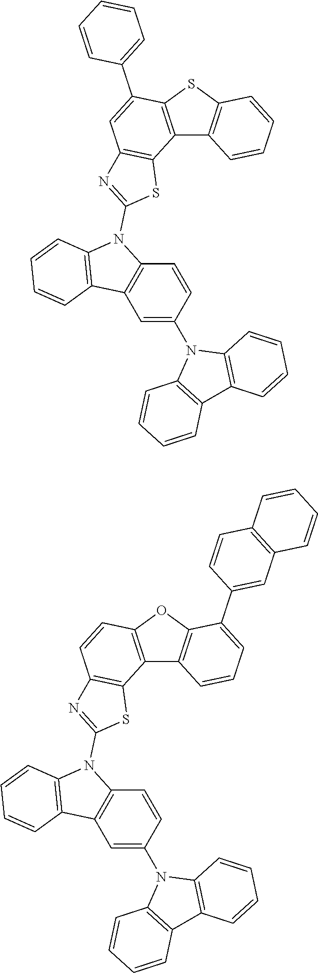 [SMILES: c1ccc(-c2cc3nc(-n4c5ccccc5c5cc(-n6c7ccccc7c7ccccc76)ccc54)sc3c3c2sc2ccccc23)cc1 ; c1ccc2cc(-c3cccc4c3oc3ccc5nc(-n6c7ccccc7c7cc(-n8c9ccccc9c9ccccc98)ccc76)sc5c34)ccc2c1]

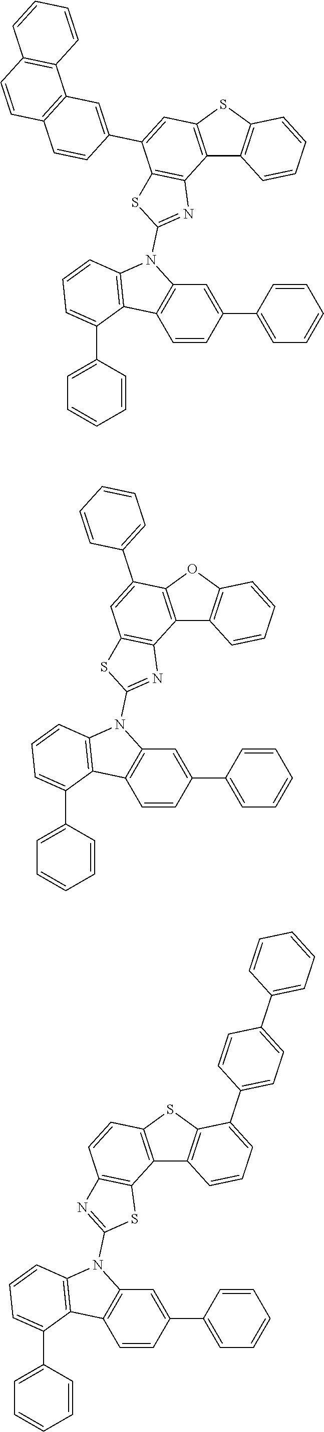 [SMILES: c1ccc(-c2ccc3c4c(-c5ccccc5)cccc4n(-c4nc5c(s4)c(-c4ccc6ccc7ccccc7c6c4)cc4sc6ccccc6c45)c3c2)cc1 ; c1ccc(-c2ccc3c4c(-c5ccccc5)cccc4n(-c4nc5c(cc(-c6ccccc6)c6oc7ccccc7c65)s4)c3c2)cc1 ; c1ccc(-c2ccc(-c3cccc4c3sc3ccc5nc(-n6c7cc(-c8ccccc8)ccc7c7c(-c8ccccc8)cccc76)sc5c34)cc2)cc1]

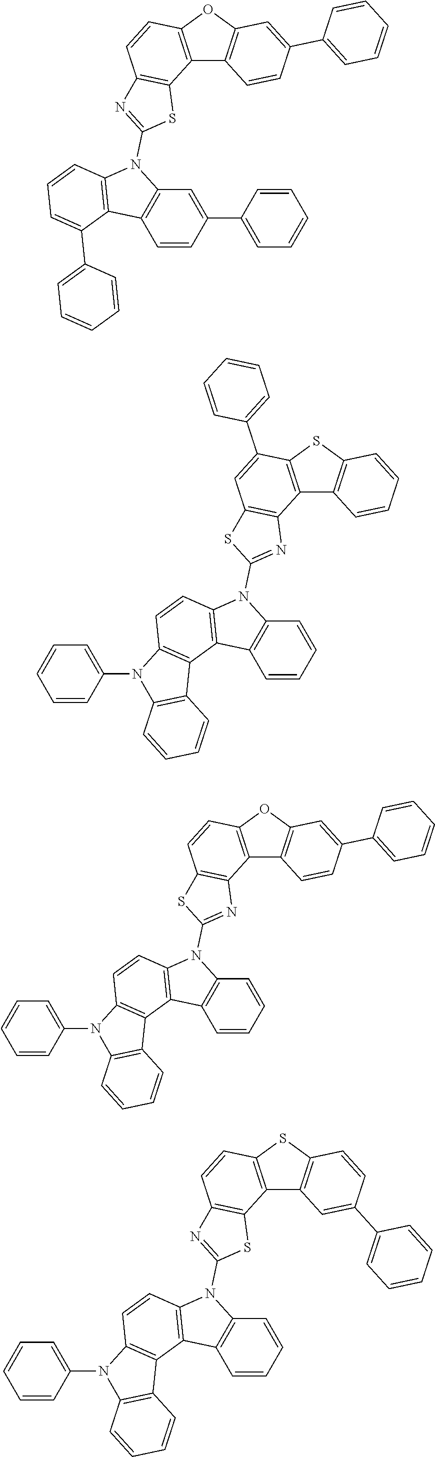 [SMILES: c1ccc(-c2ccc3c(c2)oc2ccc4nc(-n5c6cc(-c7ccccc7)ccc6c6c(-c7ccccc7)cccc65)sc4c23)cc1 ; c1ccc(-c2cc3sc(-n4c5ccccc5c5c6c7ccccc7n(-c7ccccc7)c6ccc54)nc3c3c2sc2ccccc23)cc1 ; c1ccc(-c2ccc3c(c2)oc2ccc4sc(-n5c6ccccc6c6c7c8ccccc8n(-c8ccccc8)c7ccc65)nc4c23)cc1 ; c1ccc(-c2ccc3sc4ccc5nc(-n6c7ccccc7c7c8c9ccccc9n(-c9ccccc9)c8ccc76)sc5c4c3c2)cc1]

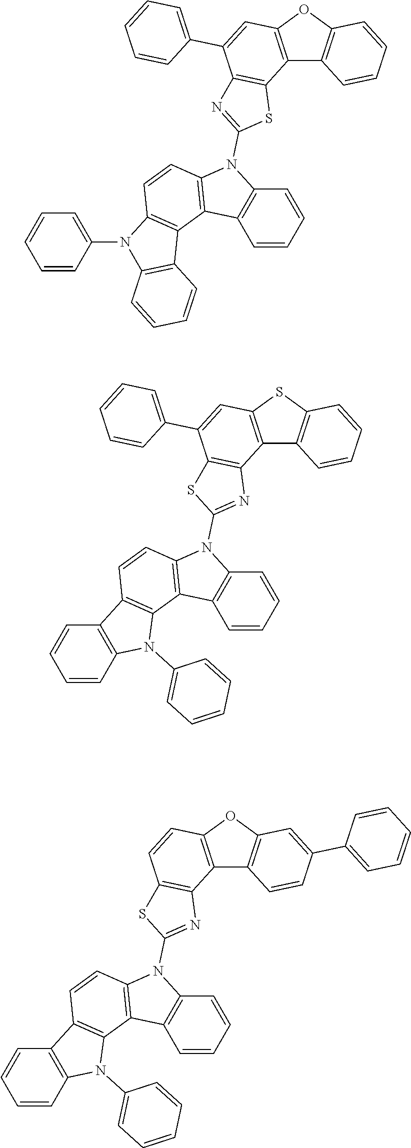 [SMILES: c1ccc(-c2cc3oc4ccccc4c3c3sc(-n4c5ccccc5c5c6c7ccccc7n(-c7ccccc7)c6ccc54)nc23)cc1 ; c1ccc(-c2cc3sc4ccccc4c3c3nc(-n4c5ccccc5c5c4ccc4c6ccccc6n(-c6ccccc6)c45)sc23)cc1 ; c1ccc(-c2ccc3c(c2)oc2ccc4sc(-n5c6ccccc6c6c5ccc5c7ccccc7n(-c7ccccc7)c56)nc4c23)cc1]

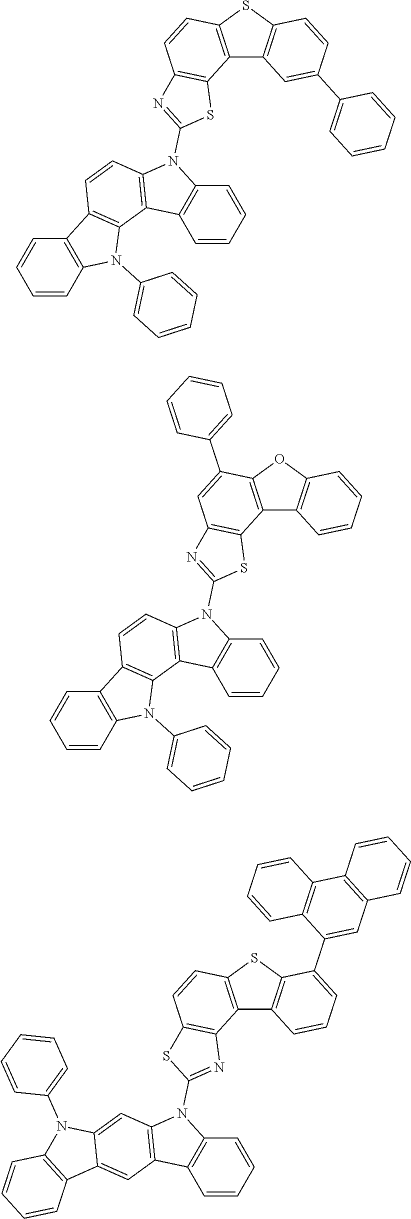 [SMILES: c1ccc(-c2ccc3sc4ccc5nc(-n6c7ccccc7c7c6ccc6c8ccccc8n(-c8ccccc8)c67)sc5c4c3c2)cc1 ; c1ccc(-c2cc3nc(-n4c5ccccc5c5c4ccc4c6ccccc6n(-c6ccccc6)c45)sc3c3c2oc2ccccc23)cc1 ; c1ccc(-n2c3ccccc3c3cc4c5ccccc5n(-c5nc6c(ccc7sc8c(-c9cc%10ccccc%10c%10ccccc9%10)cccc8c76)s5)c4cc32)cc1]

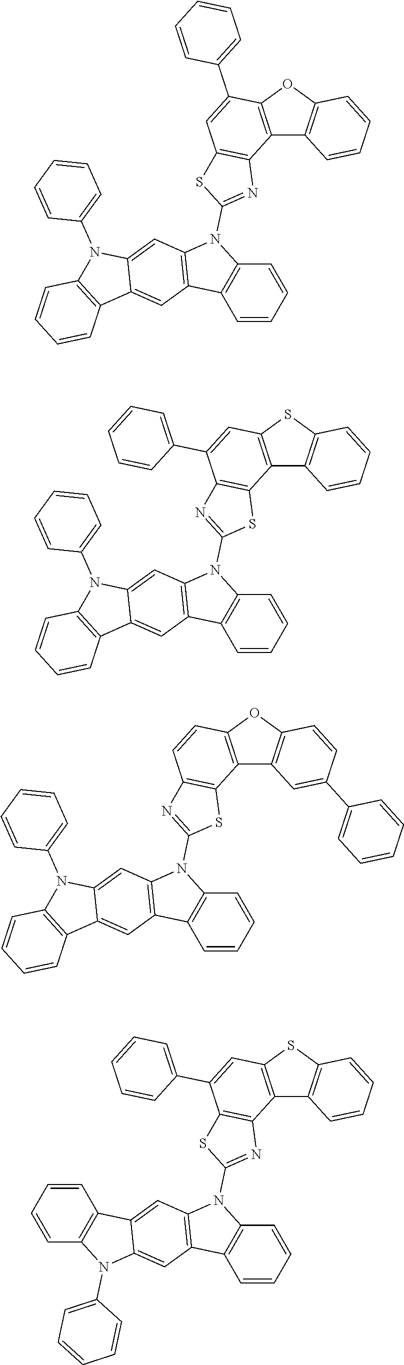 [SMILES: c1ccc(-c2cc3sc(-n4c5ccccc5c5cc6c7ccccc7n(-c7ccccc7)c6cc54)nc3c3c2oc2ccccc23)cc1 ; c1ccc(-c2cc3sc4ccccc4c3c3sc(-n4c5ccccc5c5cc6c7ccccc7n(-c7ccccc7)c6cc54)nc23)cc1 ; c1ccc(-c2ccc3oc4ccc5nc(-n6c7ccccc7c7cc8c9ccccc9n(-c9ccccc9)c8cc76)sc5c4c3c2)cc1 ; c1ccc(-c2cc3sc4ccccc4c3c3nc(-n4c5ccccc5c5cc6c(cc54)c4ccccc4n6-c4ccccc4)sc23)cc1]

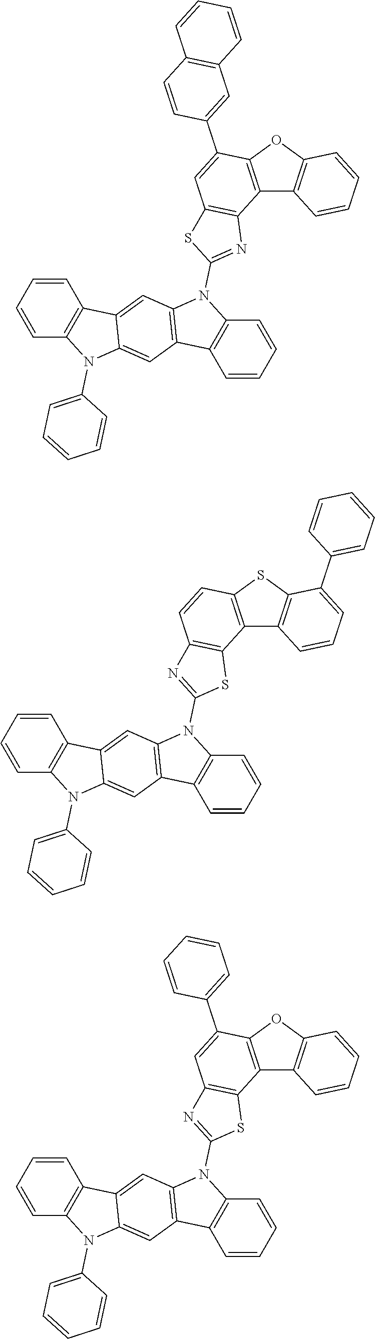 [SMILES: c1ccc(-n2c3ccccc3c3cc4c(cc32)c2ccccc2n4-c2nc3c(cc(-c4ccc5ccccc5c4)c4oc5ccccc5c43)s2)cc1 ; c1ccc(-c2cccc3c2sc2ccc4nc(-n5c6ccccc6c6cc7c(cc65)c5ccccc5n7-c5ccccc5)sc4c23)cc1 ; c1ccc(-c2cc3nc(-n4c5ccccc5c5cc6c(cc54)c4ccccc4n6-c4ccccc4)sc3c3c2oc2ccccc23)cc1]

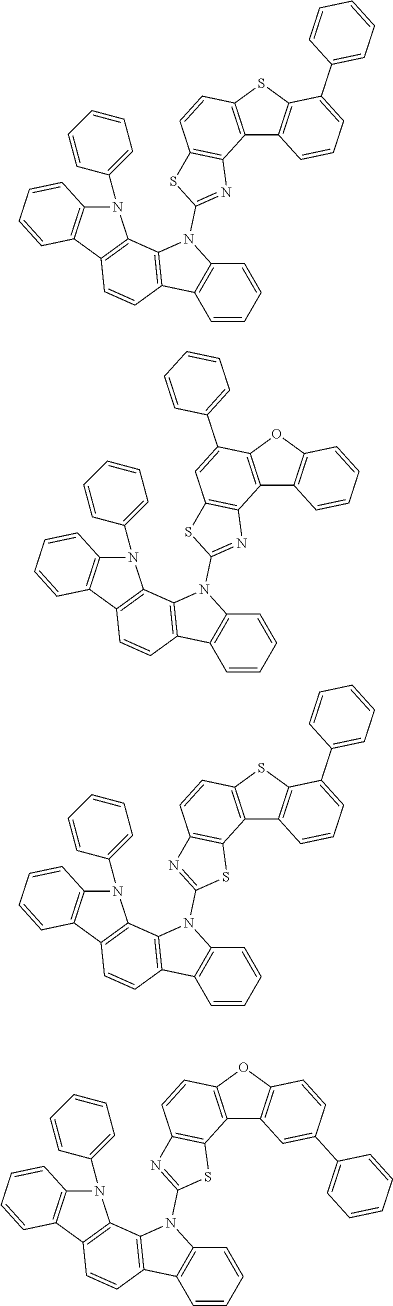

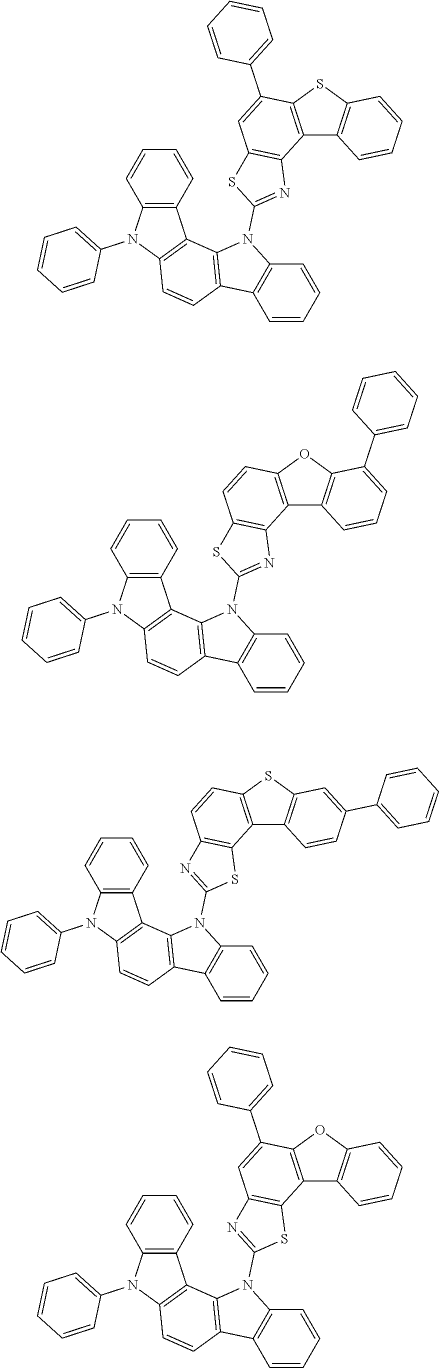 [SMILES: c1ccc(-c2cc3sc(-n4c5ccccc5c5ccc6c(c7ccccc7n6-c6ccccc6)c54)nc3c3c2sc2ccccc23)cc1 ; c1ccc(-c2cccc3c2oc2ccc4sc(-n5c6ccccc6c6ccc7c(c8ccccc8n7-c7ccccc7)c65)nc4c23)cc1 ; c1ccc(-c2ccc3c(c2)sc2ccc4nc(-n5c6ccccc6c6ccc7c(c8ccccc8n7-c7ccccc7)c65)sc4c23)cc1 ; c1ccc(-c2cc3nc(-n4c5ccccc5c5ccc6c(c7ccccc7n6-c6ccccc6)c54)sc3c3c2oc2ccccc23)cc1]

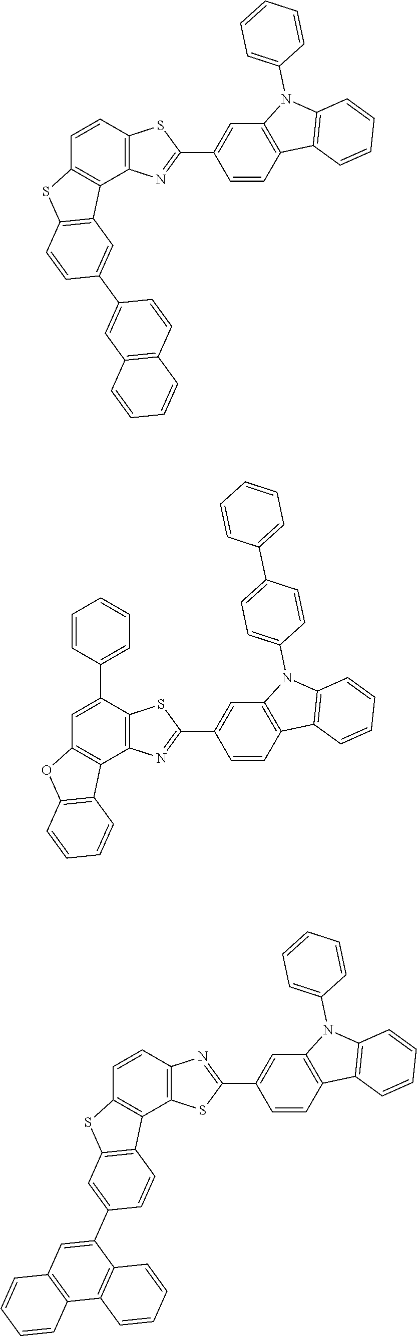 [SMILES: c1ccc(-n2c3ccccc3c3ccc(-c4nc5c(ccc6sc7ccc(-c8ccc9ccccc9c8)cc7c65)s4)cc32)cc1 ; c1ccc(-c2ccc(-n3c4ccccc4c4ccc(-c5nc6c(s5)c(-c5ccccc5)cc5oc7ccccc7c56)cc43)cc2)cc1 ; c1ccc(-n2c3ccccc3c3ccc(-c4nc5ccc6sc7cc(-c8cc9ccccc9c9ccccc89)ccc7c6c5s4)cc32)cc1]

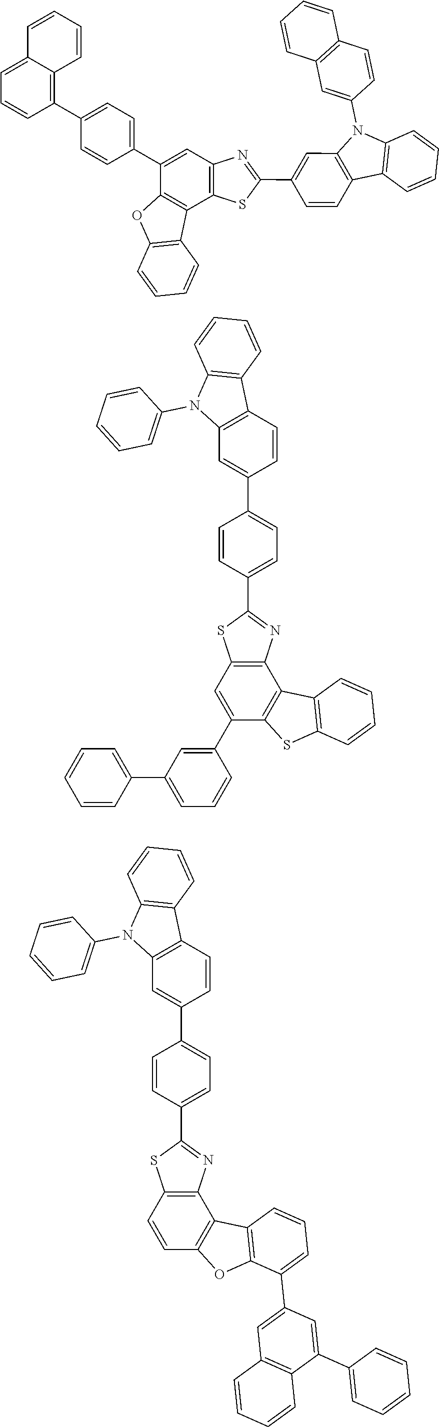

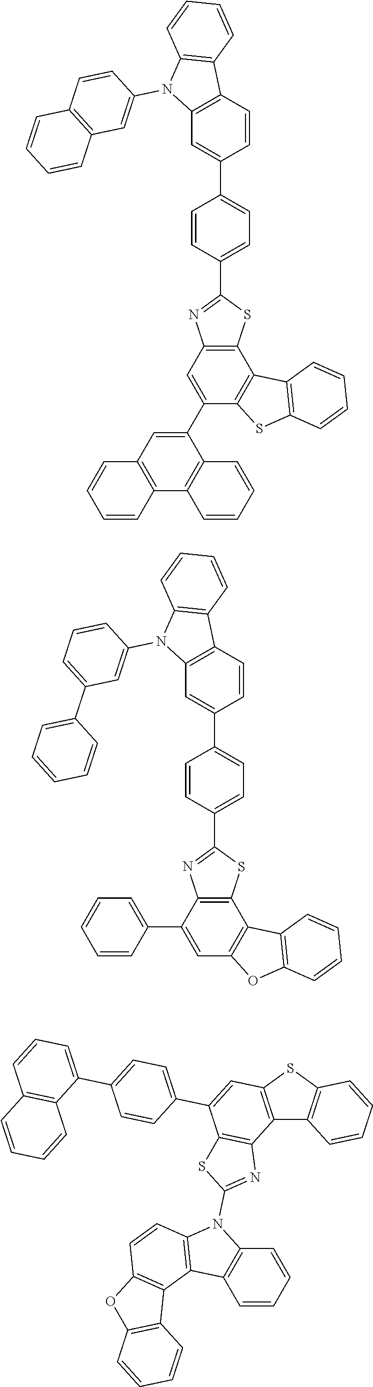

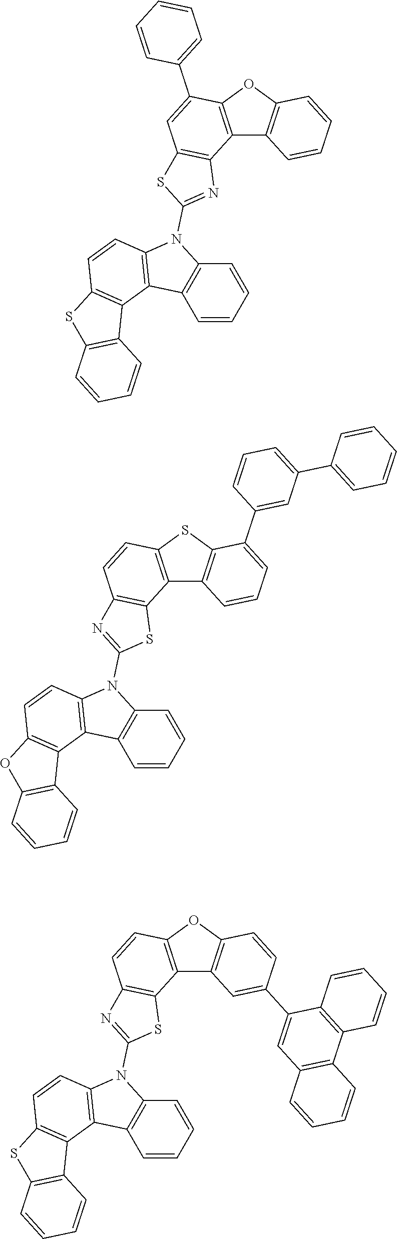 [SMILES: c1ccc(-c2cc3sc(-n4c5ccccc5c5c6c(ccc54)sc4ccccc46)nc3c3c2oc2ccccc23)cc1 ; c1ccc(-c2cccc(-c3cccc4c3sc3ccc5nc(-n6c7ccccc7c7c8c(ccc76)oc6ccccc68)sc5c34)c2)cc1 ; c1ccc2c(c1)cc(-c1ccc3oc4ccc5nc(-n6c7ccccc7c7c8c(ccc76)sc6ccccc68)sc5c4c3c1)c1ccccc12]

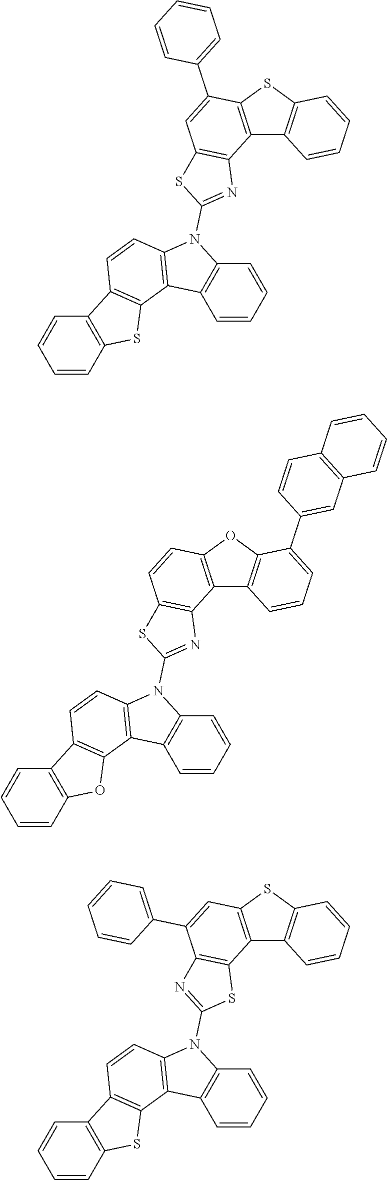 [SMILES: c1ccc(-c2cc3sc(-n4c5ccccc5c5c6sc7ccccc7c6ccc54)nc3c3c2sc2ccccc23)cc1 ; c1ccc2cc(-c3cccc4c3oc3ccc5sc(-n6c7ccccc7c7c8oc9ccccc9c8ccc76)nc5c34)ccc2c1 ; c1ccc(-c2cc3sc4ccccc4c3c3sc(-n4c5ccccc5c5c6sc7ccccc7c6ccc54)nc23)cc1]

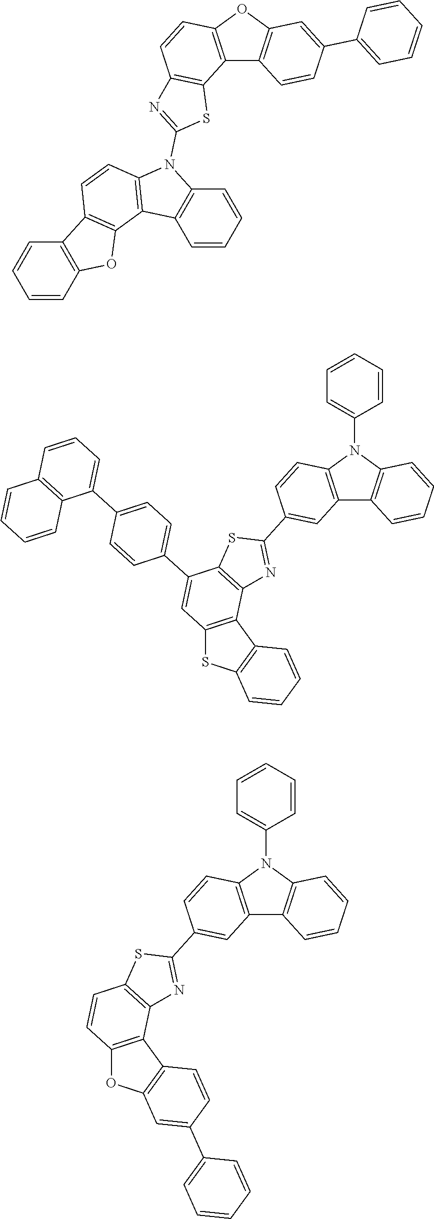 [SMILES: c1ccc(-c2ccc3c(c2)oc2ccc4nc(-n5c6ccccc6c6c7oc8ccccc8c7ccc65)sc4c23)cc1 ; c1ccc(-n2c3ccccc3c3cc(-c4nc5c(s4)c(-c4ccc(-c6cccc7ccccc67)cc4)cc4sc6ccccc6c45)ccc32)cc1 ; c1ccc(-c2ccc3c(c2)oc2ccc4sc(-c5ccc6c(c5)c5ccccc5n6-c5ccccc5)nc4c23)cc1]

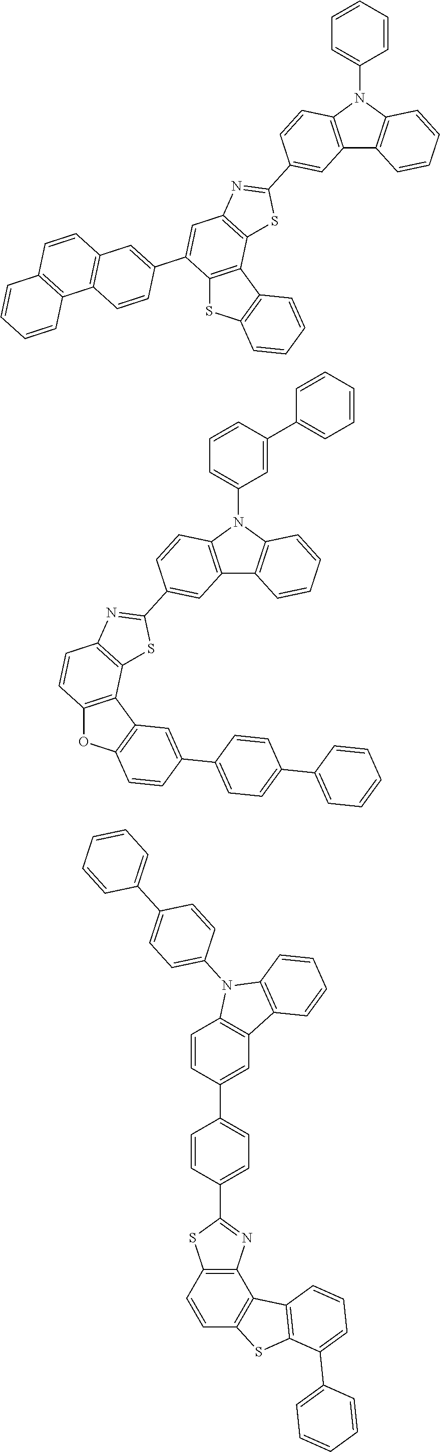

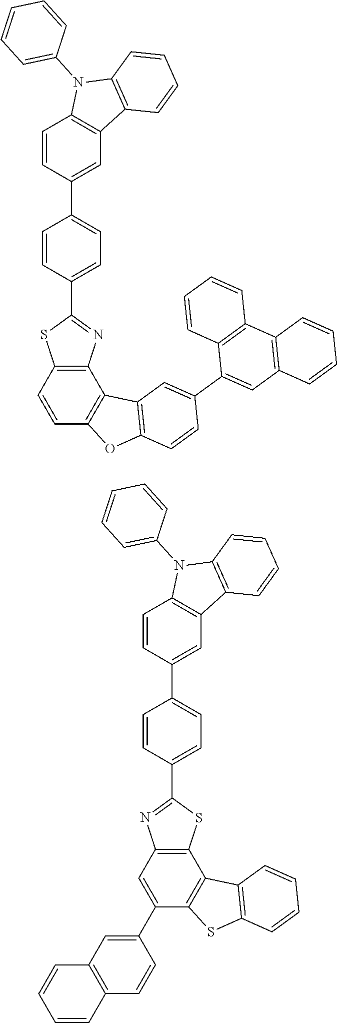

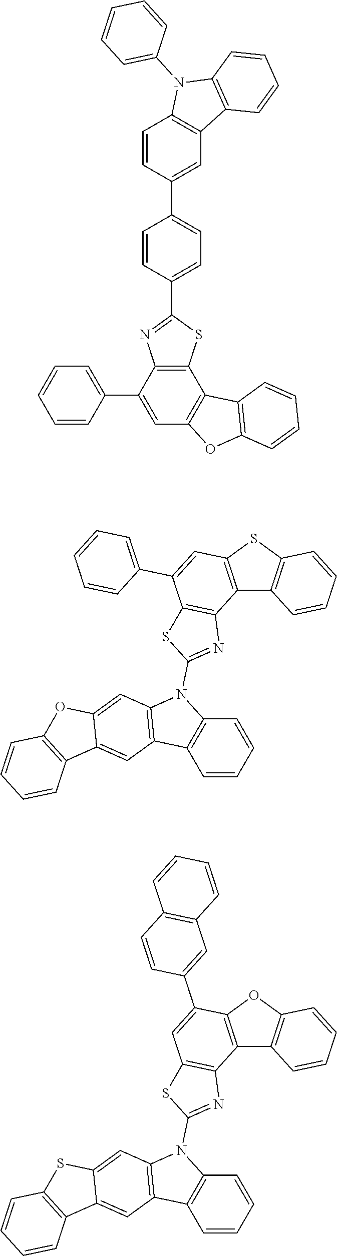 [SMILES: c1ccc(-c2cc3oc4ccccc4c3c3sc(-c4ccc(-c5ccc6c(c5)c5ccccc5n6-c5ccccc5)cc4)nc23)cc1 ; c1ccc(-c2cc3sc4ccccc4c3c3nc(-n4c5ccccc5c5cc6c(cc54)oc4ccccc46)sc23)cc1 ; c1ccc2cc(-c3cc4sc(-n5c6ccccc6c6cc7c(cc65)sc5ccccc57)nc4c4c3oc3ccccc34)ccc2c1]

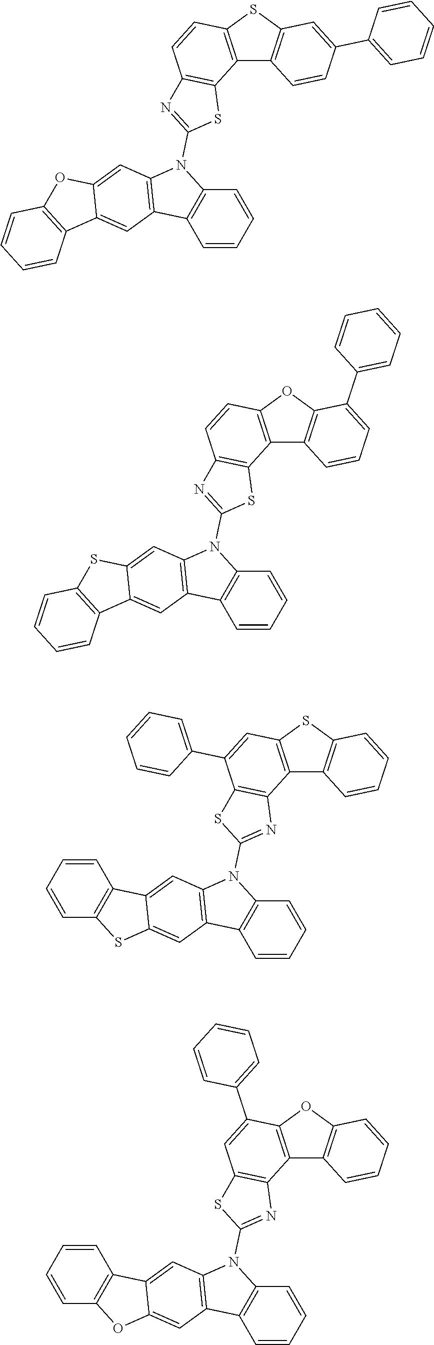 [SMILES: c1ccc(-c2ccc3c(c2)sc2ccc4nc(-n5c6ccccc6c6cc7c(cc65)oc5ccccc57)sc4c23)cc1 ; c1ccc(-c2cccc3c2oc2ccc4nc(-n5c6ccccc6c6cc7c(cc65)sc5ccccc57)sc4c23)cc1 ; c1ccc(-c2cc3sc4ccccc4c3c3nc(-n4c5ccccc5c5cc6sc7ccccc7c6cc54)sc23)cc1 ; c1ccc(-c2cc3sc(-n4c5ccccc5c5cc6oc7ccccc7c6cc54)nc3c3c2oc2ccccc23)cc1]

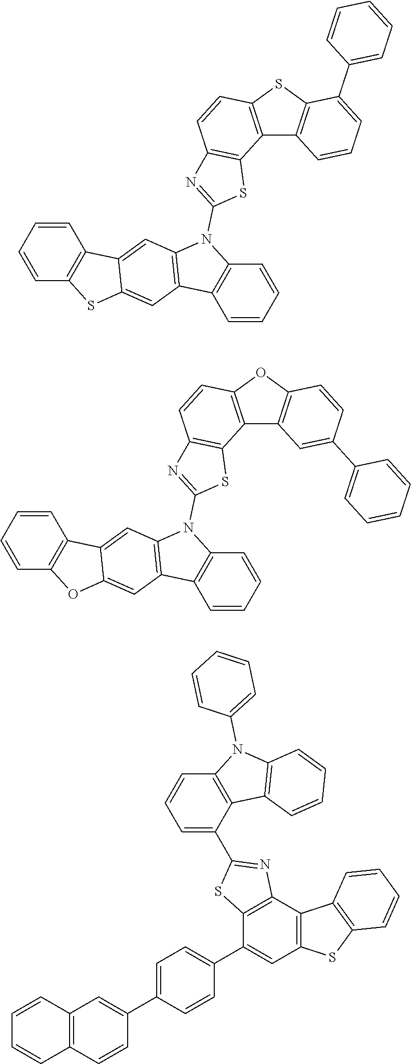

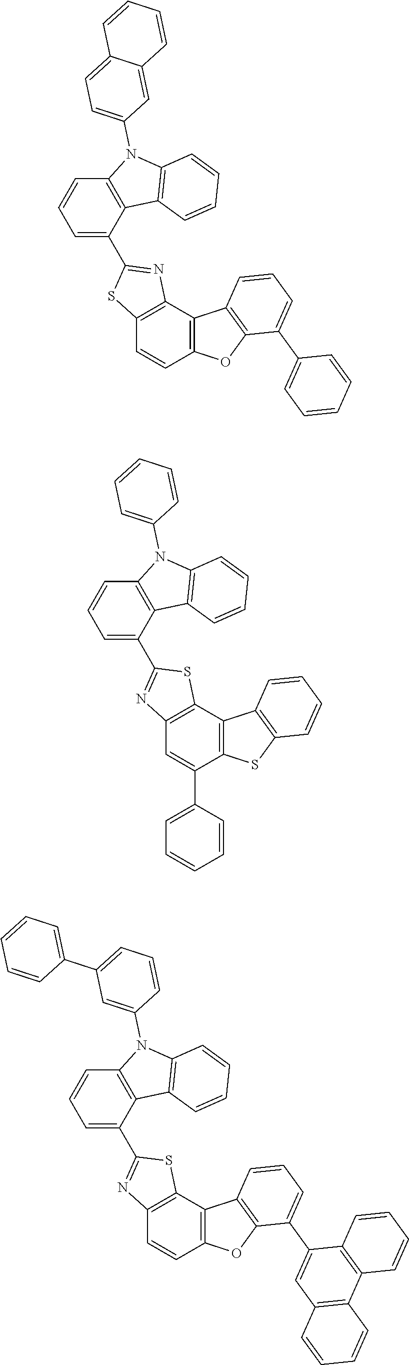 [SMILES: c1ccc(-c2cccc3c2oc2ccc4sc(-c5cccc6c5c5ccccc5n6-c5ccc6ccccc6c5)nc4c23)cc1 ; c1ccc(-c2cc3nc(-c4cccc5c4c4ccccc4n5-c4ccccc4)sc3c3c2sc2ccccc23)cc1 ; c1ccc(-c2cccc(-n3c4ccccc4c4c(-c5nc6ccc7oc8c(-c9cc%10ccccc%10c%10ccccc9%10)cccc8c7c6s5)cccc43)c2)cc1]

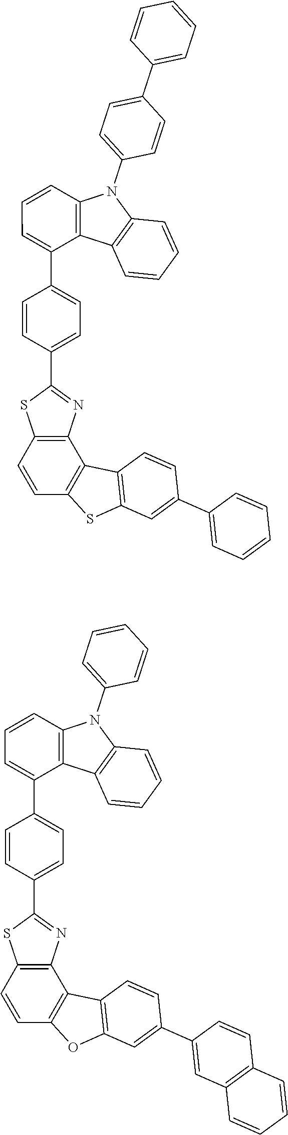 [SMILES: c1ccc(-c2ccc(-n3c4ccccc4c4c(-c5ccc(-c6nc7c(ccc8sc9cc(-c%10ccccc%10)ccc9c87)s6)cc5)cccc43)cc2)cc1 ; c1ccc(-n2c3ccccc3c3c(-c4ccc(-c5nc6c(ccc7oc8cc(-c9ccc%10ccccc%10c9)ccc8c76)s5)cc4)cccc32)cc1]

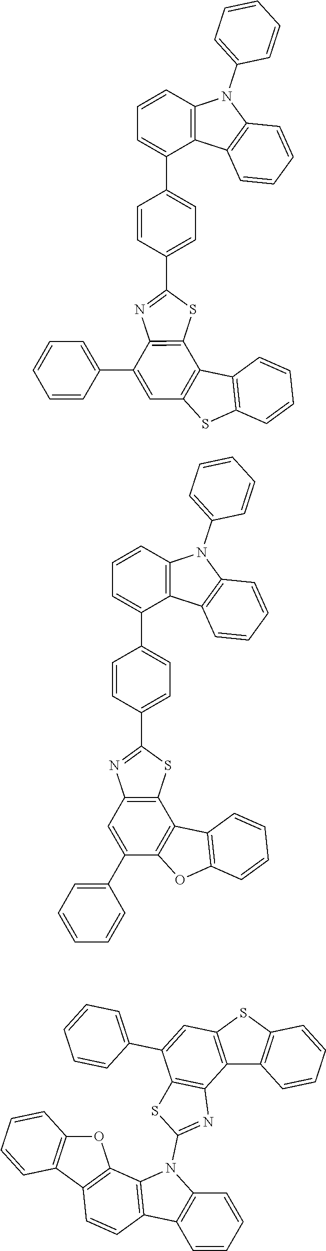

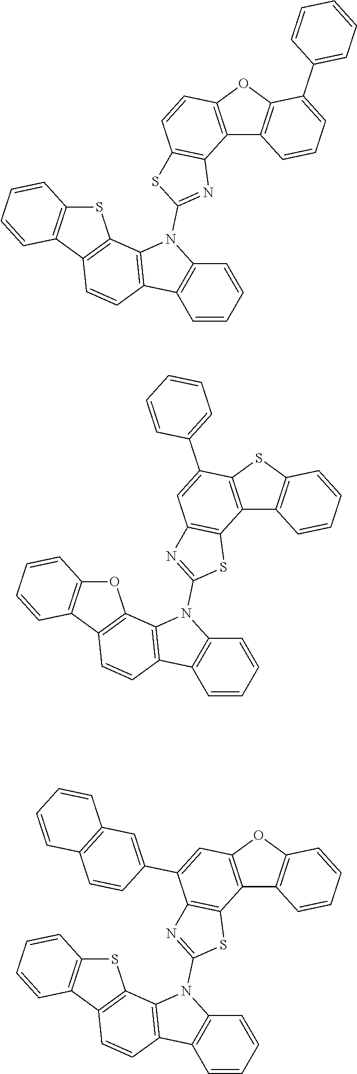 [SMILES: c1ccc(-c2cccc3c2oc2ccc4sc(-n5c6ccccc6c6ccc7c8ccccc8sc7c65)nc4c23)cc1 ; c1ccc(-c2cc3nc(-n4c5ccccc5c5ccc6c7ccccc7oc6c54)sc3c3c2sc2ccccc23)cc1 ; c1ccc2cc(-c3cc4oc5ccccc5c4c4sc(-n5c6ccccc6c6ccc7c8ccccc8sc7c65)nc34)ccc2c1]

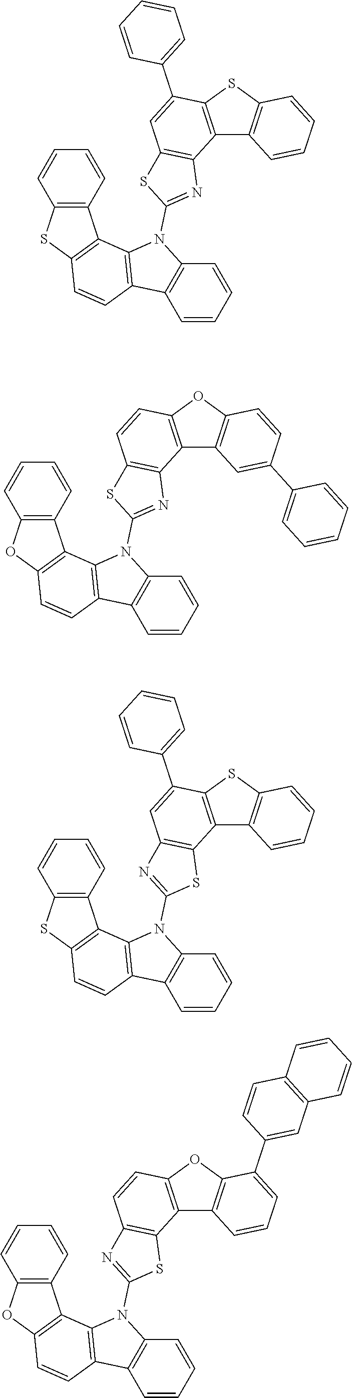 [SMILES: c1ccc(-c2cc3sc(-n4c5ccccc5c5ccc6sc7ccccc7c6c54)nc3c3c2sc2ccccc23)cc1 ; c1ccc(-c2ccc3oc4ccc5sc(-n6c7ccccc7c7ccc8oc9ccccc9c8c76)nc5c4c3c2)cc1 ; c1ccc(-c2cc3nc(-n4c5ccccc5c5ccc6sc7ccccc7c6c54)sc3c3c2sc2ccccc23)cc1 ; c1ccc2cc(-c3cccc4c3oc3ccc5nc(-n6c7ccccc7c7ccc8oc9ccccc9c8c76)sc5c34)ccc2c1]

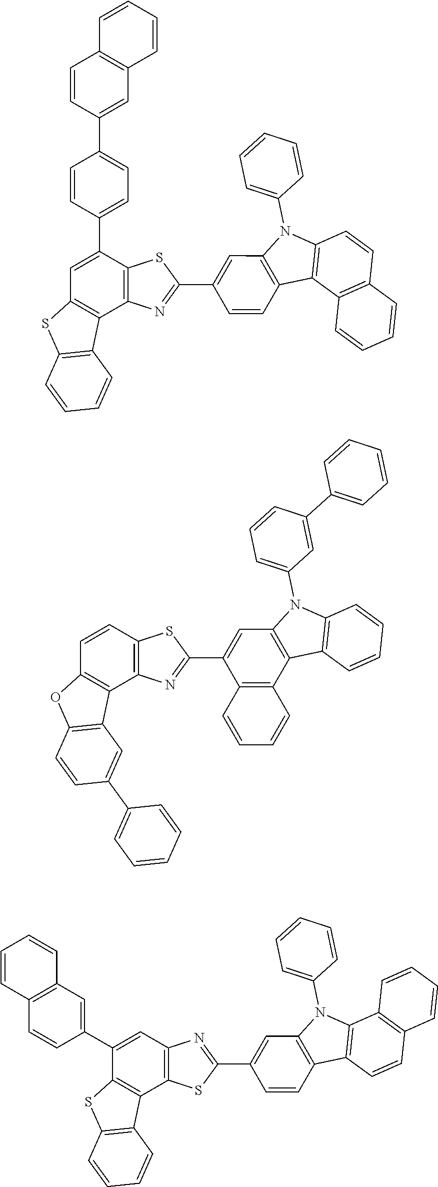 [SMILES: c1ccc(-n2c3cc(-c4nc5c(s4)c(-c4ccc(-c6ccc7ccccc7c6)cc4)cc4sc6ccccc6c45)ccc3c3c4ccccc4ccc32)cc1 ; c1ccc(-c2cccc(-n3c4ccccc4c4c5ccccc5c(-c5nc6c(ccc7oc8ccc(-c9ccccc9)cc8c76)s5)cc43)c2)cc1 ; c1ccc(-n2c3cc(-c4nc5cc(-c6ccc7ccccc7c6)c6sc7ccccc7c6c5s4)ccc3c3ccc4ccccc4c32)cc1]

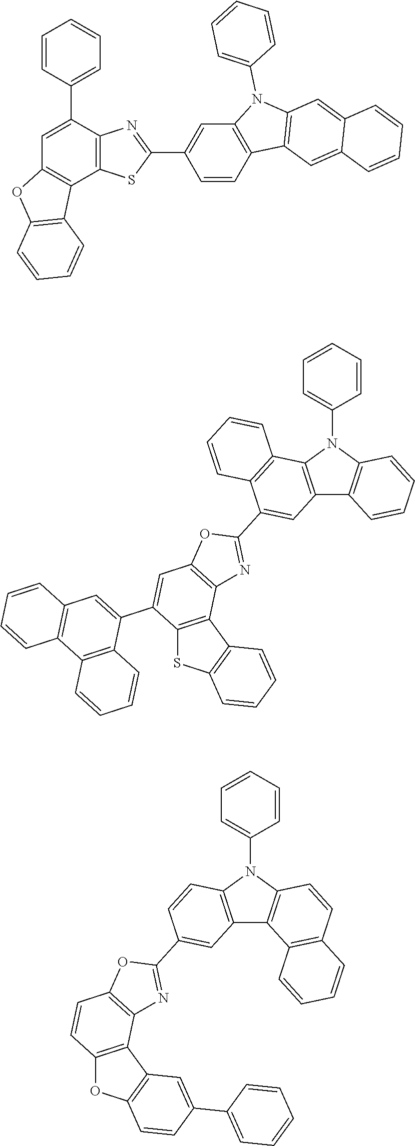 [SMILES: c1ccc(-c2cc3oc4ccccc4c3c3sc(-c4ccc5c6cc7ccccc7cc6n(-c6ccccc6)c5c4)nc23)cc1 ; c1ccc(-n2c3ccccc3c3cc(-c4nc5c(cc(-c6cc7ccccc7c7ccccc67)c6sc7ccccc7c65)o4)c4ccccc4c32)cc1 ; c1ccc(-c2ccc3oc4ccc5oc(-c6ccc7c(c6)c6c8ccccc8ccc6n7-c6ccccc6)nc5c4c3c2)cc1]

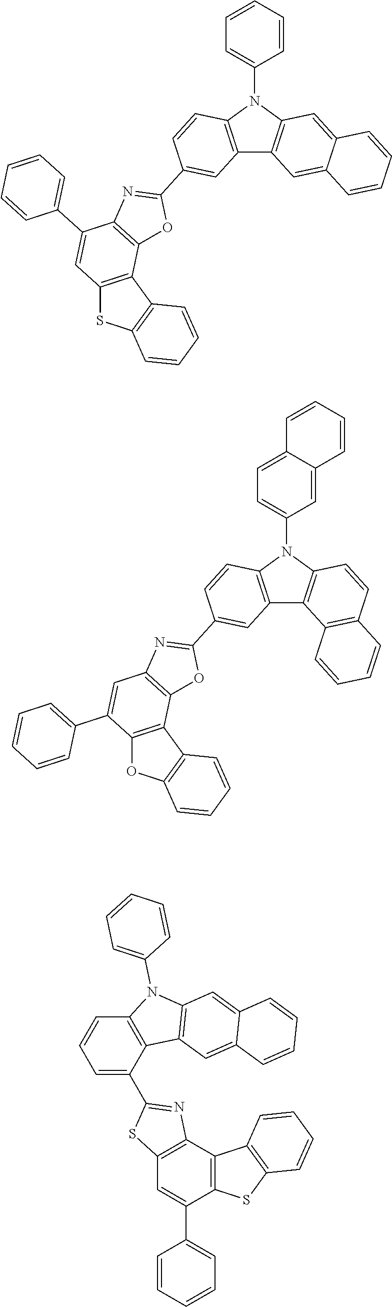 [SMILES: c1ccc(-c2cc3sc4ccccc4c3c3oc(-c4ccc5c(c4)c4cc6ccccc6cc4n5-c4ccccc4)nc23)cc1 ; c1ccc(-c2cc3nc(-c4ccc5c(c4)c4c6ccccc6ccc4n5-c4ccc5ccccc5c4)oc3c3c2oc2ccccc23)cc1 ; c1ccc(-c2cc3sc(-c4cccc5c4c4cc6ccccc6cc4n5-c4ccccc4)nc3c3c2sc2ccccc23)cc1]

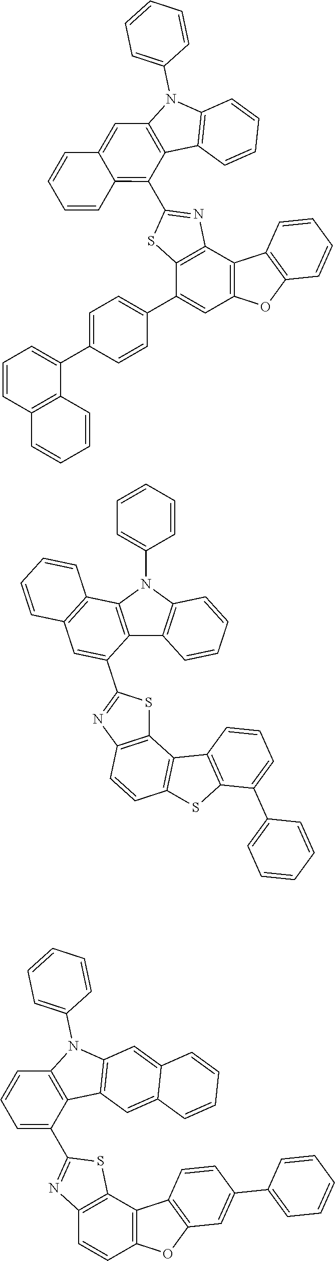 [SMILES: c1ccc(-n2c3ccccc3c3c(-c4nc5c(s4)c(-c4ccc(-c6cccc7ccccc67)cc4)cc4oc6ccccc6c45)c4ccccc4cc32)cc1 ; c1ccc(-c2cccc3c2sc2ccc4nc(-c5cc6ccccc6c6c5c5ccccc5n6-c5ccccc5)sc4c23)cc1 ; c1ccc(-c2ccc3c(c2)oc2ccc4nc(-c5cccc6c5c5cc7ccccc7cc5n6-c5ccccc5)sc4c23)cc1]

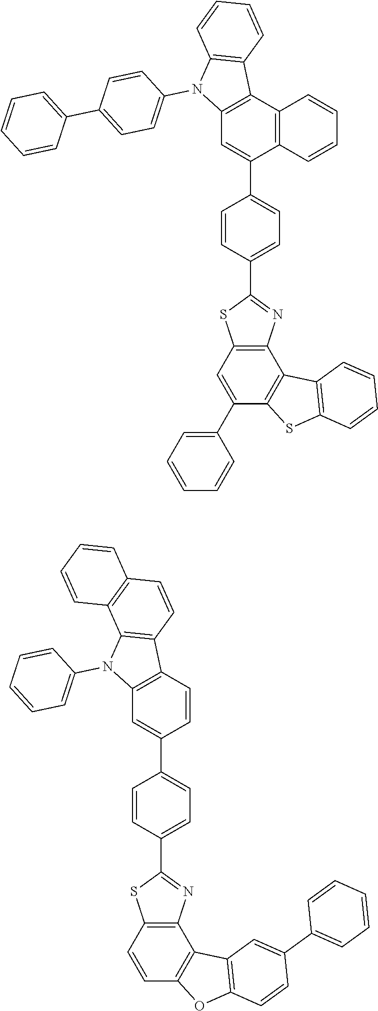 [SMILES: c1ccc(-c2ccc(-n3c4ccccc4c4c5ccccc5c(-c5ccc(-c6nc7c(cc(-c8ccccc8)c8sc9ccccc9c87)s6)cc5)cc43)cc2)cc1 ; c1ccc(-c2ccc3oc4ccc5sc(-c6ccc(-c7ccc8c9ccc%10ccccc%10c9n(-c9ccccc9)c8c7)cc6)nc5c4c3c2)cc1]

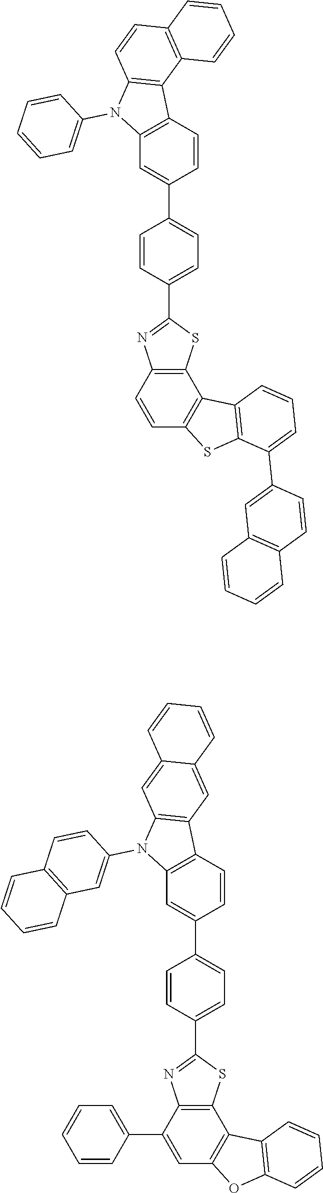 [SMILES: c1ccc(-n2c3cc(-c4ccc(-c5nc6ccc7sc8c(-c9ccc%10ccccc%10c9)cccc8c7c6s5)cc4)ccc3c3c4ccccc4ccc32)cc1 ; c1ccc(-c2cc3oc4ccccc4c3c3sc(-c4ccc(-c5ccc6c7cc8ccccc8cc7n(-c7ccc8ccccc8c7)c6c5)cc4)nc23)cc1]

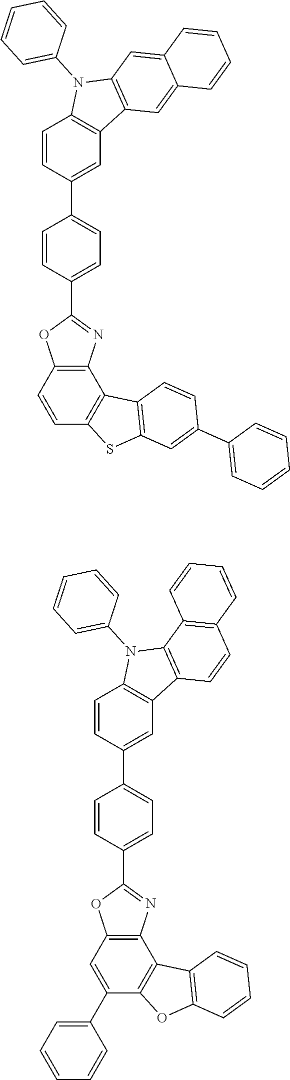 [SMILES: c1ccc(-c2ccc3c(c2)sc2ccc4oc(-c5ccc(-c6ccc7c(c6)c6cc8ccccc8cc6n7-c6ccccc6)cc5)nc4c23)cc1 ; c1ccc(-c2cc3oc(-c4ccc(-c5ccc6c(c5)c5ccc7ccccc7c5n6-c5ccccc5)cc4)nc3c3c2oc2ccccc23)cc1]

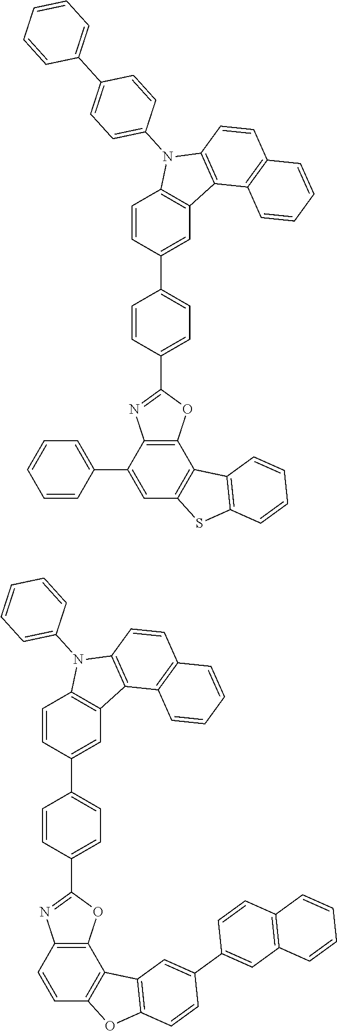 [SMILES: c1ccc(-c2ccc(-n3c4ccc(-c5ccc(-c6nc7c(-c8ccccc8)cc8sc9ccccc9c8c7o6)cc5)cc4c4c5ccccc5ccc43)cc2)cc1 ; c1ccc(-n2c3ccc(-c4ccc(-c5nc6ccc7oc8ccc(-c9ccc%10ccccc%10c9)cc8c7c6o5)cc4)cc3c3c4ccccc4ccc32)cc1]

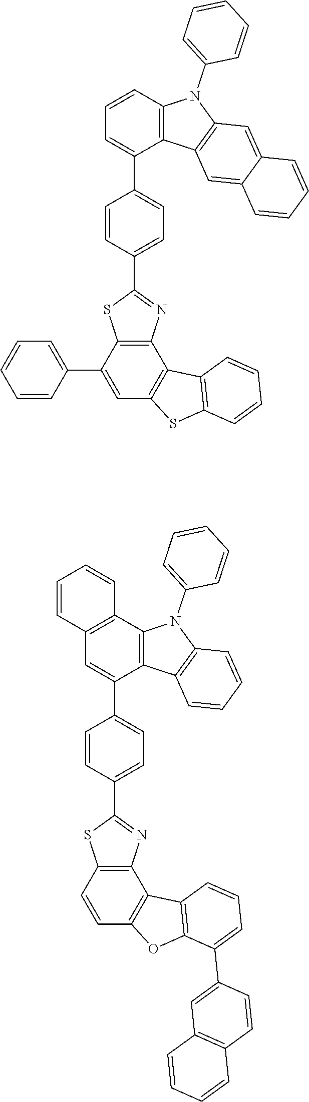 [SMILES: c1ccc(-c2cc3sc4ccccc4c3c3nc(-c4ccc(-c5cccc6c5c5cc7ccccc7cc5n6-c5ccccc5)cc4)sc23)cc1 ; c1ccc(-n2c3ccccc3c3c(-c4ccc(-c5nc6c(ccc7oc8c(-c9ccc%10ccccc%10c9)cccc8c76)s5)cc4)cc4ccccc4c32)cc1]

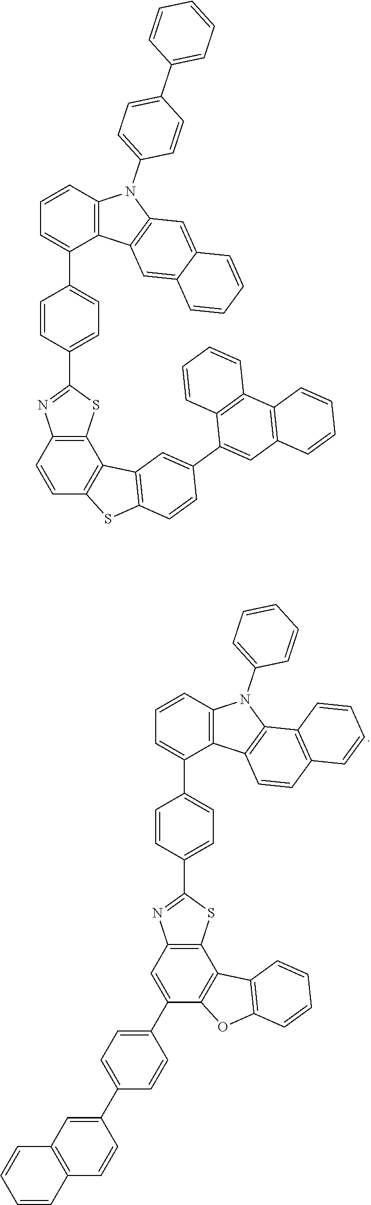 [SMILES: c1ccc(-c2ccc(-n3c4cc5ccccc5cc4c4c(-c5ccc(-c6nc7ccc8sc9ccc(-c%10cc%11ccccc%11c%11ccccc%10%11)cc9c8c7s6)cc5)cccc43)cc2)cc1 ; c1ccc(-n2c3cccc(-c4ccc(-c5nc6cc(-c7ccc(-c8ccc9ccccc9c8)cc7)c7oc8ccccc8c7c6s5)cc4)c3c3ccc4ccccc4c32)cc1]

9. An organic light emitting device, comprising:
a first electrode;
a second electrode that is opposite to the first electrode; and
one or more organic material layers that are between the first electrode and the second electrode, wherein one or more layers of the one or more organic material layers comprises the compound of claim 1.

10. The organic light emitting device of claim 9, wherein:
the organic material layer comprising the compound is a light emitting layer.
